(19) United States
(12) Patent Application Publication
(10) Pub. No.: US 2026/0008749 A1
(43) Pub. Date: Jan. 8, 2026

(54) SULFONIUM SALT, ACID DIFFUSION INHIBITOR, RESIST COMPOSITION, AND PATTERNING PROCESS

(71) Applicant: SHIN-ETSU CHEMICAL CO., LTD., Tokyo (JP)

(72) Inventors: Takayuki FUJIWARA, Joetsu-shi (JP); Miyu YOSHIDA, Joetsu-shi (JP)

(73) Assignee: SHIN-ETSU CHEMICAL CO., LTD., Tokyo (JP)

(21) Appl. No.: 19/256,447

(22) Filed: Jul. 1, 2025

(30) Foreign Application Priority Data
Jul. 4, 2024 (JP) 2024-108420

(51) Int. Cl. C07C 381/12; G03F 7/004; G03F 7/16; G03F 7/20; G03F 7/30

(52) U.S. Cl. CPC C07C 381/12; G03F 7/0045; G03F 7/168; G03F 7/20; G03F 7/30

(57) ABSTRACT

A sulfonium salt represented by the following general formula (1). In formula, R¹, R², and R³ represent a fluorine atom, a hydroxy group, a trifluoromethyl group, a trifluoromethoxy group, a trifluoroacetyloxy group, a cyano group, a carbamoyl group, an amide group, or a linear, branched, or cyclic monovalent hydrocarbon group having 1 to 15 carbon atoms and optionally having a heteroatom; “r” represents integer of 0 to 4, “p” represents integer of 0 to 5, “q” represents integer of 0 to 5, “x” represents integer of 0 to 4, “y” represents integer of 0 to 5, “z” represents integer of 0 to 5, and “r”, “p”, “q”, “x”, “y”, and “z” satisfy x+y+z≥1, 4≥r+x≥0, 5≥p+y≥0, and 5≥q+z≥0. This can provide an acid diffusion inhibitor used for a chemically-amplified resist material that does not impair sensitivity in photolithography and is excellent in lithography performances such as CDU and LWR.

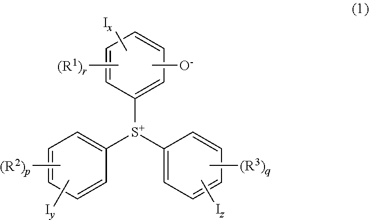

### TECHNICAL FIELD

The present invention relates to a sulfonium salt, an acid diffusion inhibitor, a resist composition, and a patterning process.

### BACKGROUND ART

As higher integration and higher speed of LSI have been achieved in recent years, the pattern rule has been required to be miniaturized, and a resist pattern is required to have high resolution. Accordingly, in addition to lithography characteristics represented by pattern shape, contrast, Mask Error Factor (MEF), Depth of Focus (DOF), Line Width Roughness (LWR), or the like, there is an increasing need to improve defects in a resist pattern after development.

In particular, as a pattern is miniaturized, the line width roughness (LWR) of a line pattern and the critical dimension uniformity (CDU) of a hole pattern are regarded as a problem. It is pointed out that these properties are possibly influenced by biased distribution or aggregation of a base polymer or an acid generator and by acid diffusion. Further, as thinner resist films are formed, LWR tends to increase. The film thickness reduction due to progress in the miniaturization causes LWR degradations, resulting in serious problems.

In a resist composition for EUV-lithography, it is necessary to simultaneously achieve sensitivity enhancement, resolution enhancement, and lowering of LWR. When acid diffusion length is shortened, LWR decreases, but the sensitivity is reduced. For example, lowering the post-exposure bake (PEB) temperature decreases LWR, but reduces the sensitivity. Increasing the amount of quencher added also reduces LWR, but reduces the sensitivity. It is necessary to overcome the trade-off relationship between the sensitivity and LWR.

It has been confirmed an effect that an iodine atom has a very large absorption of EUV light having a wavelength of 13.5 nm and thus generates secondary electrons during exposure to the light, and this effect has attracted attention in EUV-lithography fields. Patent Documents 1 and 2 describe a photo-acid generator and an acid diffusion inhibitor in which an iodine atom is introduced into an anion, and Patent Documents 3 and 4 describe a photo-acid generator and an acid diffusion inhibitor in which an iodine atom is introduced into a cation. Although it has been confirmed that these improve lithography performance to a certain extent, the iodine atom is not highly soluble in organic solvents so that there are concerns about the risk of precipitation in a solvent and a pattern defect.

In order to overcome the trade-off between sensitivity and LWR, various additives have been investigated. These include structure optimization of a photo-acid generator, an amine, and an acid diffusion inhibitor of a weak acid onium salt-type, and sensitivity enhancement by adding an acid amplifiers. Patent Document 5 discloses investigation of an onium salt-type acid diffusion inhibitors having both a cationic moiety and an anionic moiety on the same molecule. However, a resist material that simultaneously satisfies the requirements for sensitivity, LWR, and CDU has not yet been developed.

### CITATION LIST

### Patent Literature

Patent Document 1: JP6720926B2
Patent Document 2: JP6702264B2
Patent Document 3: JP7140075B2
Patent Document 4: JP7099418B2
Patent Document 5: JP6848776B2

### SUMMARY OF INVENTION

### Technical Problem

For the recent year demand for a resist pattern having high resolution, a resist material using a conventional acid diffusion inhibitor does not always satisfactory in terms of lithography performances such as CDU and LWR.

The present invention was made in view of the above circumstances, and an object of the present invention is to provide: an acid diffusion inhibitor used for a chemically-amplified resist material that does not impair sensitivity in photolithography using a high-energy beam as a light source and is excellent in lithography performances such as LWR; a chemically-amplified resist material containing the acid diffusion inhibitor; and a patterning process using the resist material.

### Solution to Problem

To solve the above problems, the present invention provides a sulfonium salt represented by the following general formula (1),

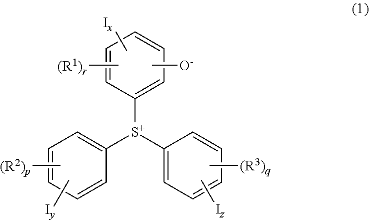

wherein R¹, R², and R³ each independently represent a fluorine atom, a hydroxy group, a trifluoromethyl group, a trifluoromethoxy group, a trifluoroacetyloxy group, a cyano group, a carbamoyl group, an amide group, or a linear, branched, or cyclic monovalent hydrocarbon group having 1 to 15 carbon atoms and optionally having a heteroatom, and a methylene group in the hydrocarbon group may be replaced with an ether bond (—O—) or a carbonyl group (—CO—); R¹ and R² may be bonded to each other to form a ring together with a benzene ring and a sulfur atom, R² and R³ may be bonded to each other to form a ring together with a benzene ring and a sulfur atom, and when the ring is formed, R¹ and R², and R² and R³ may combine to form a single bond, a divalent group, or a divalent atom; “r” represents an integer of 0 to 4, “p” represents an integer of 0 to 5, “q” represents an integer of 0 to 5, “x” represents an integer of 0 to 4, “y” represents an integer of 0 to 5, “z” represents an integer of 0 to 5, and “r”, “p”, “q”, “x”, “y”, and “z” satisfy x+y+z≥1, 4≥r+x≥0, 5≥p+y≥0, and 5≥q+z≥0; R²s may be the same or different from each other when r≥2, R²s may be the same or different from each other when p≥2, and R³s may be the same or different from each other when q≥2.

Such a sulfonium salt does not impair sensitivity in photolithography using high-energy beam such as a KrF excimer laser beam, an ArF excimer laser beam, an EB, and EUV light as a light source, and is an acid diffusion inhibitor that can be used in a chemically-amplified resist material excellent in lithography performances such as CDU and LWR.

Further, the general formula (1) preferably satisfies x≥1.

In this event, the general formula (1) preferably satisfies y=0 and z=0.

An iodine atom and a phenoxy group are present on the same benzene ring, so that decomposition product having an iodine atom produced by light exposure is hydrophilic and thus reduces a risk of a residual defect.

Further, the sulfonium salt is preferably represented by the following general formula (1-A),

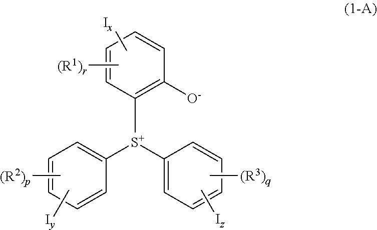

wherein R¹, R², R³, “r”, “p”, “q”, “x”, “y”, and “z” are the same as defined above.

When a phenoxy group is present at the ortho position of S⁺, the cationic part and the anionic part have a three-dimensionally close structure, which allows the formation of an electronically neutral structure within a single molecule, and thus it is promising to improve storage stability and organic solvent solubility of the compound.

Further, preferably, R¹, R², and R³ each independently represents a group selected from the group consisting of a fluorine atom, a hydroxy group, a cyano group, a carbamoyl group, or an alkyl group having 1 to 5 carbon atoms and optionally having a heteroatom, an alkoxy group having 1 to 5 carbon atoms and optionally having a heteroatom, and an alkylcarbonyloxy group having 1 to 5 carbon atoms and optionally having a heteroatom.

Each benzene ring in the formula may have such a substituent.

Further, the present invention provides an acid diffusion inhibitor comprising the sulfonium salt above.

The inventive sulfonium salt may be used particularly suitably as an acid diffusion inhibitor.

Further, the present invention provides a resist composition comprising a resin component (component A) that changes its solubility in a developer by an action of an acid, a photo-acid generator (component B), an organic solvent (component D), and the acid diffusion inhibitor (component C-1) above.

By using the inventive acid diffusion inhibitor for a resist composition, it is possible to increase lithography performances such as CDU and LWR without impairing sensitivity in photolithography.

Further, the present invention provides a resist composition comprising: a resin component (component A-1) that generates an acid by exposure to light and changes its solubility in a developer by an action of an acid; an organic solvent (component D); and the acid diffusion inhibitor (component C-1) above.

Instead of adding a photo-acid generator, the resin component may have a property of generating an acid by exposure to light.

Further, the resin component (component A) or the resin component (component A-1) may have a repeating unit (P-1) having a phenolic hydroxy group.

In this event, the repeating unit (P-1) is preferably represented by the following general formula (5),

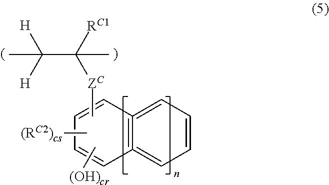

wherein R^{C1} represents a hydrogen atom or a methyl group; Z^{C} represents a single bond or an ester bond; R^{C2} represents a fluorine atom, an iodine atom, a trifluoromethyl group, a trifluoromethoxy group, or a monovalent organic group having 1 to 10 carbon atoms and optionally having a heteroatom, and “—CH₂—” in the organic group may be substituted with “—O—”, or “—C(═O)—”; cs represents an integer of 0 to 4; cr represents an integer of 1 to 5; and “n” represents 0 or 1.

In the inventive resist composition, the resin component may be such.

Further, the inventive resist composition may comprise an acid diffusion inhibitor (component C-2), other than the acid diffusion inhibitor (component C-1).

The inventive resist composition may be added with any acid diffusion inhibitor, without limiting to the inventive acid diffusion inhibitor.

Further, the inventive resist composition is preferably used to form an image by exposure with an electron beam or EUV light.

The inventive resin composition may be particularly suitably used for exposure with an electron beam or EUV light.

Further, the present invention provides a patterning process comprising;
applying the resist composition above onto a substrate; heat-treating the resist composition applied on the substrate;
exposing the resist composition to light of a KrF excimer laser beam, an ArF excimer laser beam, an electron beam, or EUV light; and developing the resist composition with a developer.

The inventive patterning process can improve lithography performance such as CDU or LWR in photolithography using a KrF excimer laser beam, an ArF excimer laser beam, an EB, or EUV light as a light source without impairing sensitivity.

Optionally, an exposed portion is dissolved by using an alkali aqueous solution as the developer, and an unexposed portion is not dissolved to obtain a positive pattern.

Alternatively, an unexposed portion is dissolved by using an organic solvent as the developer, and an exposed portion is not dissolved to obtain a negative pattern.

The inventive patterning process can form both positive and negative pattern.

### Advantageous Effects of Invention

When a pattern is formed using a resist composition in which the inventive sulfonium salt is incorporated as an acid diffusion inhibitor, it is possible to form a pattern that is excellent in lithography performance such as CDU and LWR without impairing sensitivity.

### BRIEF DESCRIPTION OF DRAWINGS

FIG. 1 is a nuclear magnetic resonance spectrum of the sulfonium salt synthesized in Example 1-1.

### DESCRIPTION OF EMBODIMENTS

As described above, it has been desired to develop: an acid diffusion inhibitor used for a chemically-amplified resist material that does not impair sensitivity in photolithography using a high-energy beam such as a KrF excimer laser beam, an ArF excimer laser beam, an EB, and EUV light as a light source and is excellent in lithography performances such as CDU and LWR; a chemically-amplified resist material containing the acid diffusion inhibitor; and a patterning process using the resist material.

The present inventors have earnestly studied to achieve the above objectives and found out that a resist material using a sulfonium salt having a certain structure as an acid diffusion inhibitor was excellent in lithography performance such as CDU and LWR and was extremely effective as a resist material for fine and precise processing, and have completed the present invention.

That is, the present invention is a sulfonium salt represented by the general formula (1) above.

Hereinafter, the present invention will be described in detail. In the following description, some structures represented by chemical formulae may have asymmetric carbons and may have an enantiomer or a diastereomer, in which case a single formula will be used to represent all of the isomers. One of these isomers may be used or a mixture thereof may be used.

### [Sulfonium Salt]

The inventive sulfonium salt is represented by the following general formula (1).

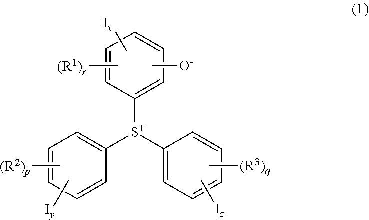

In the formula, R¹, R², and R³ each independently represent a fluorine atom, a hydroxy group, a trifluoromethyl group, a trifluoromethoxy group, a trifluoroacetyloxy group, a cyano group, a carbamoyl group, an amide group, or a linear, branched, or cyclic monovalent hydrocarbon group having 1 to 15 carbon atoms and optionally having a heteroatom, and a methylene group in the hydrocarbon group may be replaced with an ether bond (—O—) or a carbonyl group (—CO—); R¹ and R² may be bonded to each other to form a ring together with a benzene ring and a sulfur atom, R² and R³ may be bonded to each other to form a ring together with a benzene ring and a sulfur atom, and when the ring is formed, R¹ and R², and R² and R³ may combine to form a single bond, a divalent group, or a divalent atom; “r” represents an integer of 0 to 4, “p” represents an integer of 0 to 5, “q” represents an integer of 0 to 5, “x” represents an integer of 0 to 4, “y” represents an integer of 0 to 5, “z” represents an integer of 0 to 5, and “r”, “p”, “q”, “x”, “y”, and “z” satisfy x+y+z≥1, 4≥r+x≥0, 5≥p+y≥0, and 5≥q+z≥0; R¹s may be the same or different from each other when r≥2, R²s may be the same or different from each other when p≥2, and R³s may be the same or different from each other when q≥2.

Specifically, R¹, R², and R³ are exemplified as following structures. Examples thereof include a fluorine atom, a hydroxy group, a methoxy group, an ethoxymethoxy group, a trifluoromethyl group, a trifluoromethoxy group, a trifluoroacetyloxy group, an acetyl group, a carbamoyl group, an amido group, a cyano group, a methyl group, an ethyl group, a propyl group, an isopropyl group, an n-butyl group, a sec-butyl group, a tert-butyl group, an n-pentyl group, an n-hexyl group, an n-octyl group, an n-nonyl group, an n-decyl group, a cyclopentyl group, a cyclohexyl group, an adamantyl group, a 1-adamantanemethyl group, a phenyl group, and a phenoxy group. Furthermore, a part of the carbon atoms and the hydrogen atoms in these groups may be substituted with a heteroatom such as an oxygen atom, a sulfur atom, a nitrogen atom, and a halogen atom, or a heteroatom may intervene, optionally resulting in formation or intervention of a hydroxyl group, a cyano group, a carbonyl group, an ether bond, an ester bond, a sulfonate ester bond, a carbonate bond, a lactone ring, a sultone ring, a carboxylic acid anhydride, a haloalkyl group, etc. As the ring formed by R¹ and R² bonding to each other together with a benzene ring and a sulfur atom, and by R² and R³ bonding to each other together with a benzene ring and a sulfur atom, the following structures are exemplified. In the diagram below, the substituents on the benzene ring are omitted, but each benzene ring may have a substituent after the ring is formed.

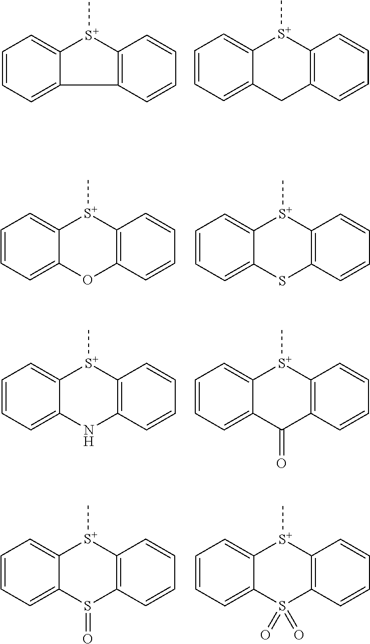

R¹, R², and R³ each independently are particularly preferably a group selected from the group consisting of a fluorine atom, a hydroxy group, a cyano group, a carbamoyl group, an alkyl group having 1 to 5 carbon atoms and optionally having a heteroatom, an alkoxy group having 1 to 5 carbon atoms and optionally having a heteroatom, and an alkylcarbonyloxy group having 1 to 5 carbon atoms and optionally having a heteroatom.

“r” represents an integer of 0 to 4, “p” represents an integer of 0 to 5, “q” represents an integer of 0 to 5, “x” represents an integer of 0 to 4, “y” represents an integer of 0 to 5, and “z” represents an integer of 0 to 5, and “r”, “p”, “q”, “x”, “y”, and “z” are integers that satisfy x+y+z≥1, 4≥r+x≥0, 5≥p+y≥0, and 5≥q+z≥0. “r” is preferably an integer from 0 to 2, “p” is preferably an integer from 0 to 2, “q” is preferably an integer from 0 to 2, “x” is preferably an integer from 1 to 3, “y” is preferably an integer from 0 to 1, and “z” is preferably an integer from 0 to 1. R's may be the same or different from each other when r≥2, R²s may be the same or different from each other when p≥2, and R³s may be the same or different from each other when q≥2.

R¹, R², and R³ preferably include a fluorine atom, a trifluoromethyl group, a trifluoromethoxy group, a hydroxy group, a methoxy group, and a tert-butoxy group, and more preferably they are a fluorine atom, a trifluoromethyl group, a trifluoromethoxy group, or a methoxy group.

The substitution position of R¹ is not particularly limited. The substitution positions of R² and R³ are preferably para- or meta-position with respect to S+, and particularly preferably meta-position. By having an electron-withdrawing group at the meta-position, the LUMO of the cation is decreased, and the acid generation efficiency is promisingly expected to be improved.

x+y+z preferably satisfy 3≥x+y+z≥1. By having an iodine atom, EUV light can be efficiently absorbed, and an improvement in sensitivity can be promisingly expected. When the number of iodine atoms is three or less, the risk that the solubility in an alkaline developer decreases due to the hydrophobicity of the iodine atom to generate a residual defect is further reduced. From the viewpoint of the balance between sensitivity enhancement and defect risks, it is preferable to have 1 to 3 iodine atoms.

“x”, “y”, and “z” each independently preferably satisfy x≥1, and particularly preferably satisfy 3≥x≥1, y=0, and z=0. By having an iodine atom and a phenoxy group on the same benzene ring, decomposition product having an iodine atom generated by exposure becomes hydrophilic, thereby reducing the risk of a residual defect.

The inventive sulfonium salt preferably has a structure represented by the following general formula (1-A).

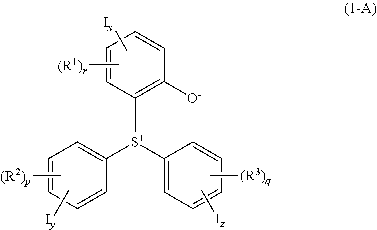

In the formula, R¹, R², R³, “r”, “p”, “q”, “x”, “y”, and “z” are the same as defined above.

When the phenoxy group is present at the ortho position of S⁺, the cationic part and the anionic part have a three-dimensionally close structure, which allows the formation of an electronically neutral structure within a single molecule, and thus it is promising to improve storage stability and organic solvent solubility of the compound.

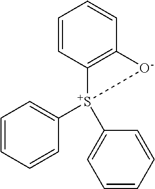

In the formula, the description of the substituents on the benzene rings is omitted.

Specific examples of the sulfonium salt represented by the formula (1) include those shown below, but are not limited thereto.

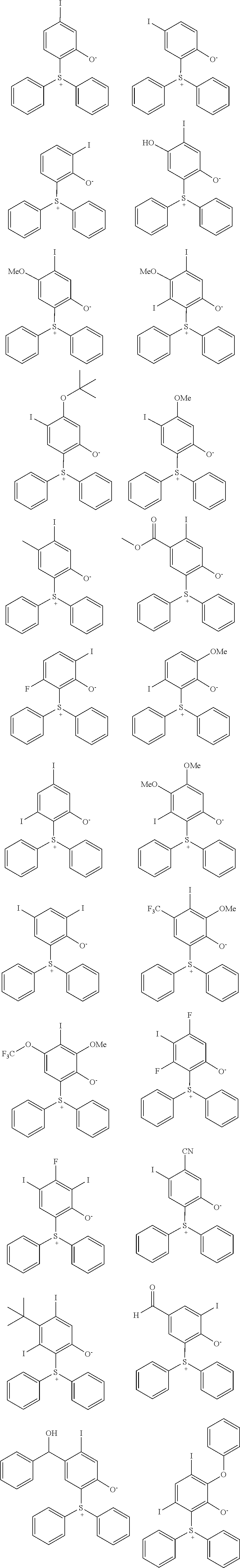

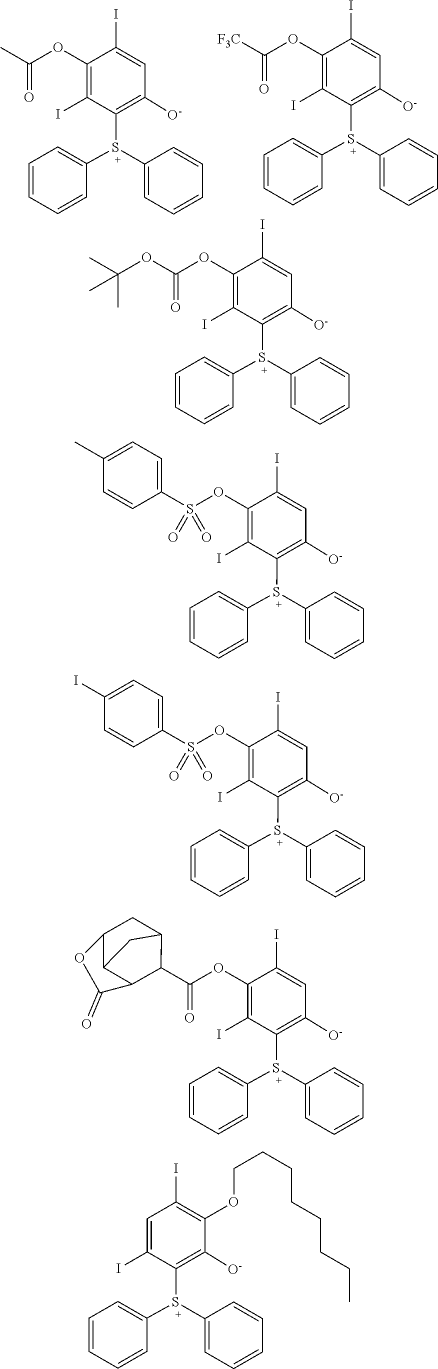

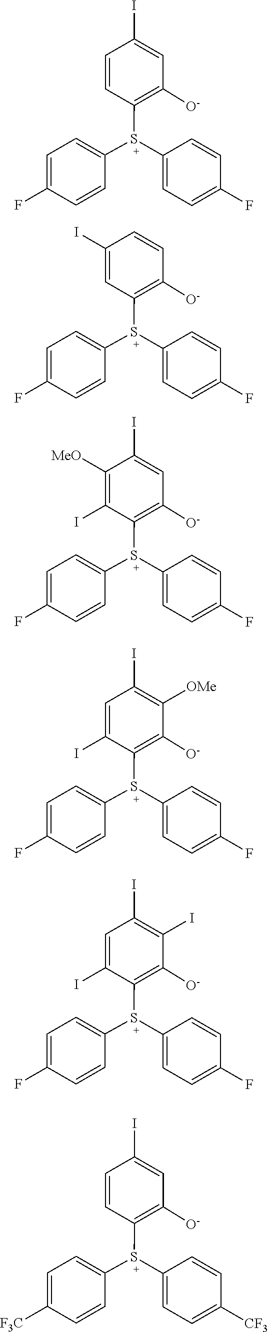

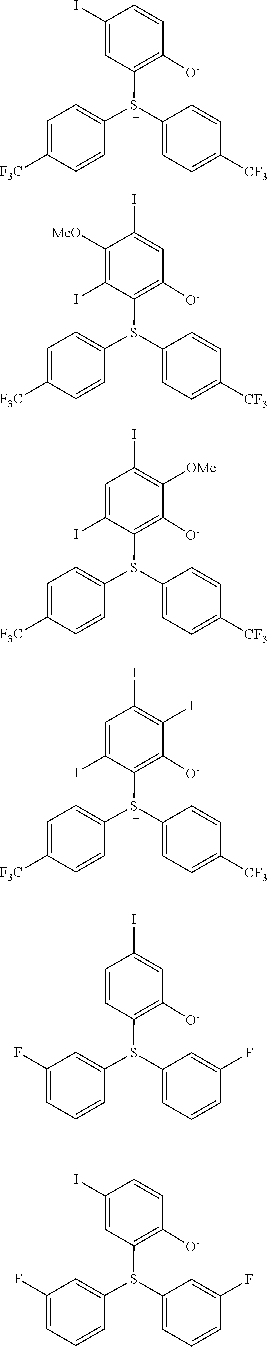

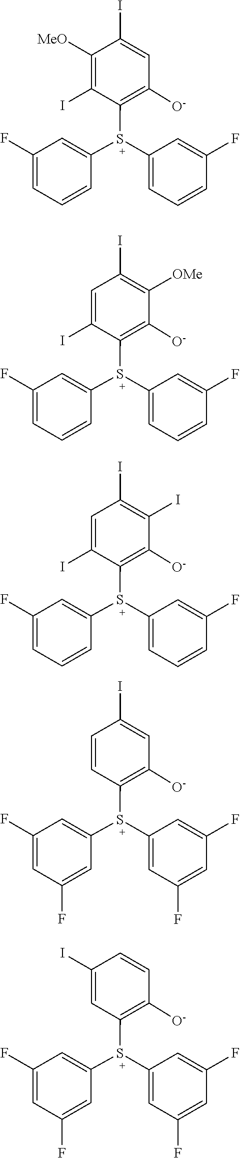

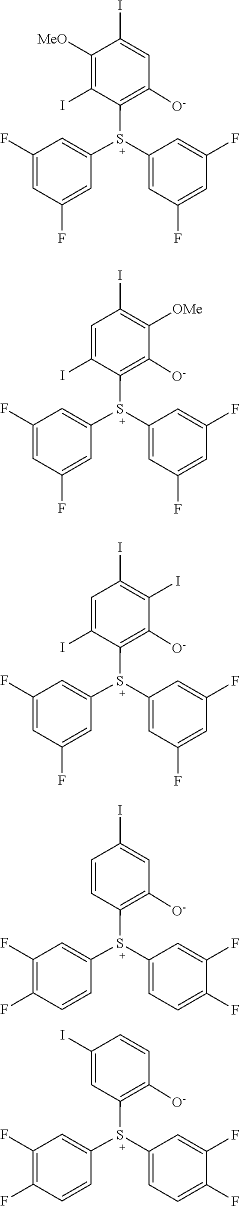

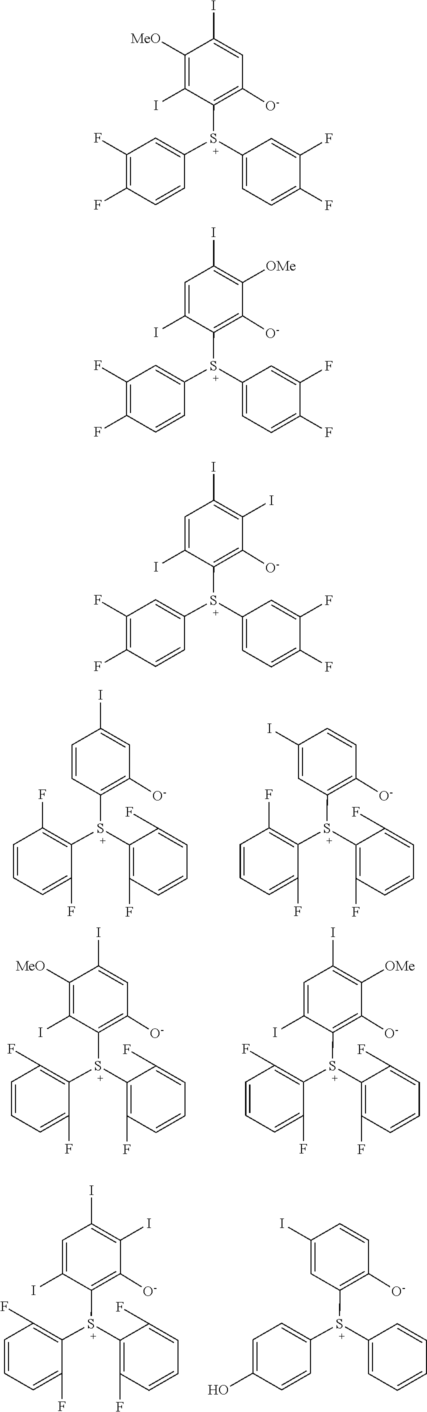

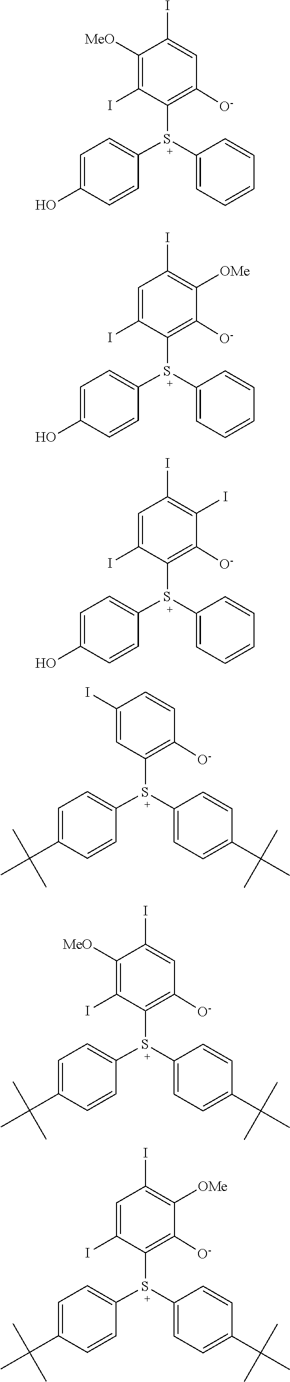

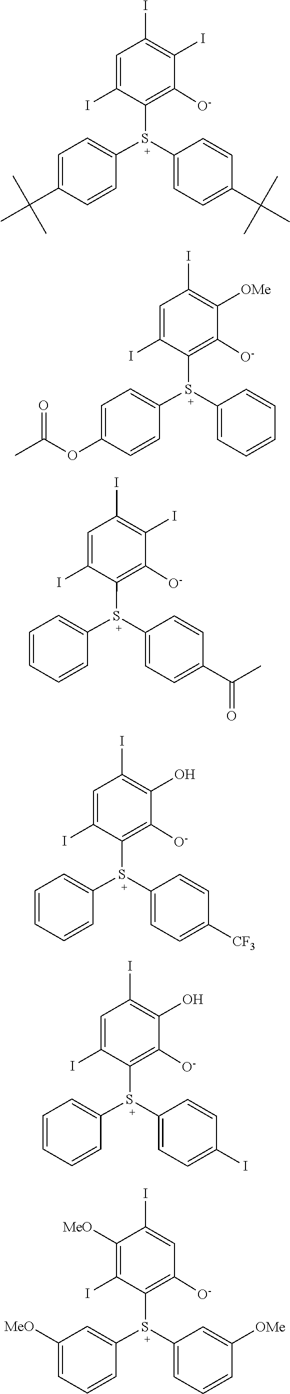

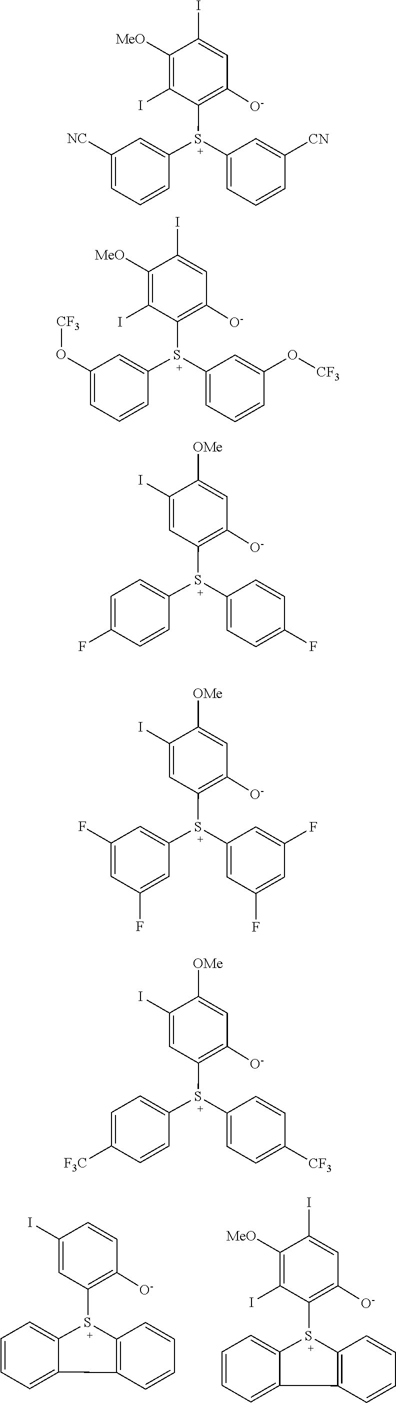

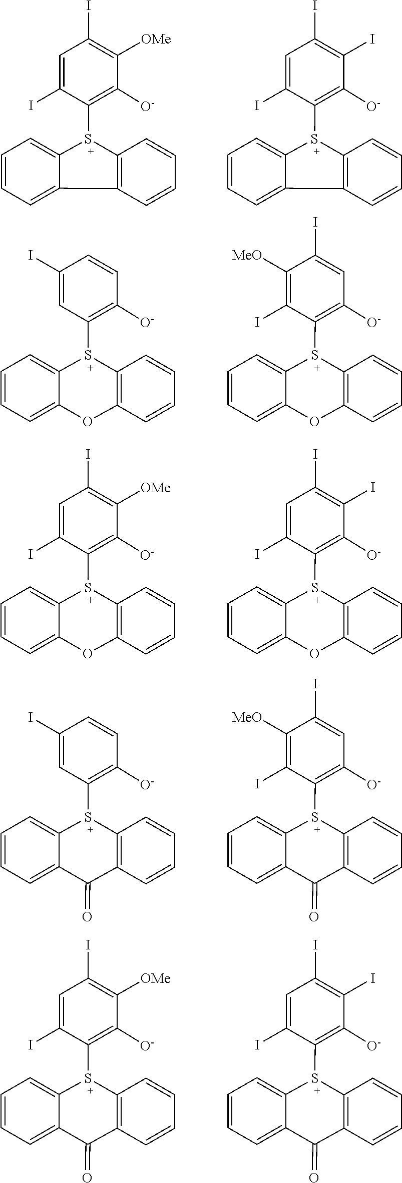

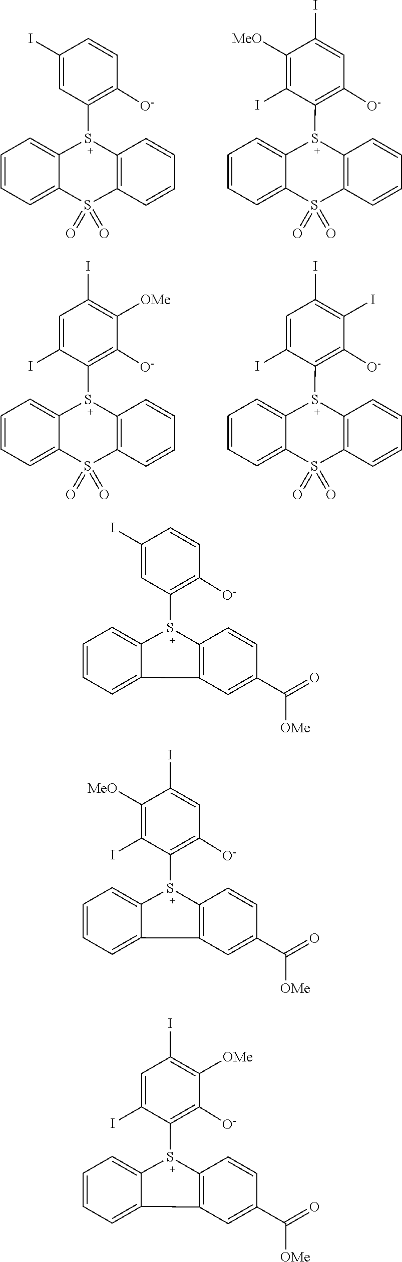

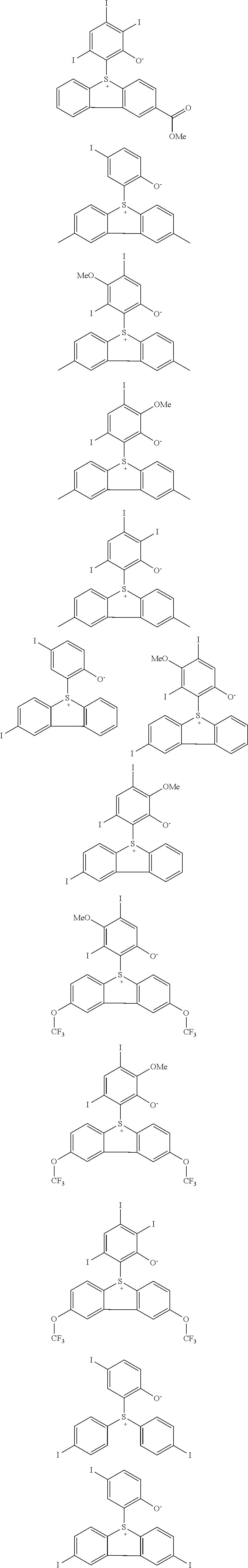

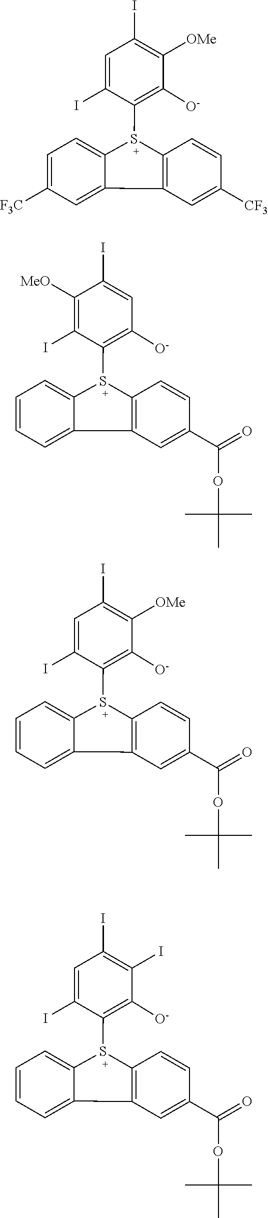

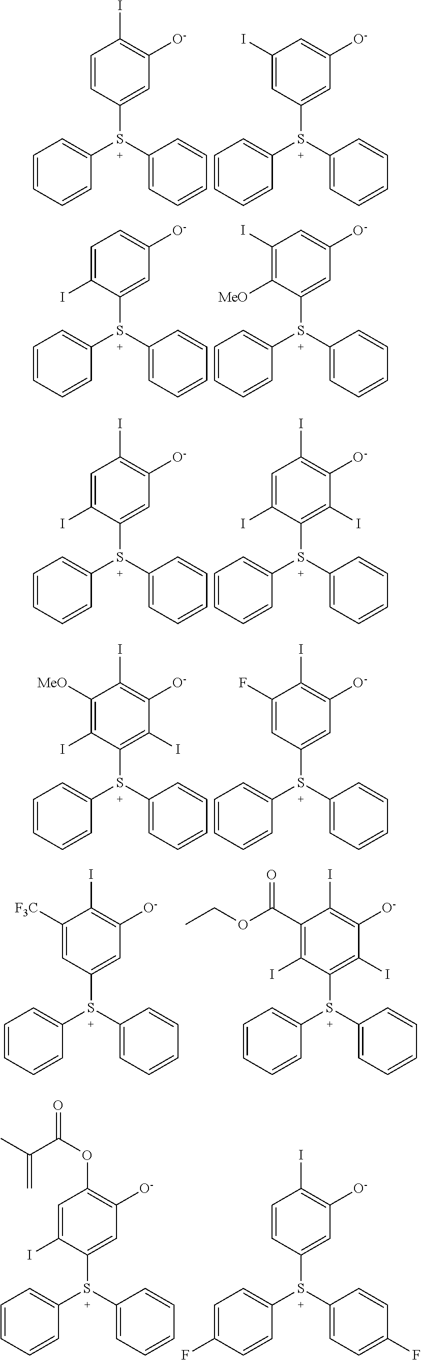

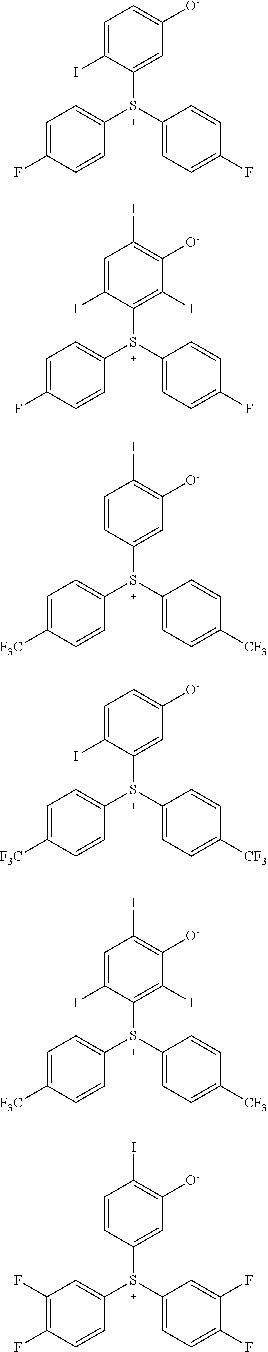

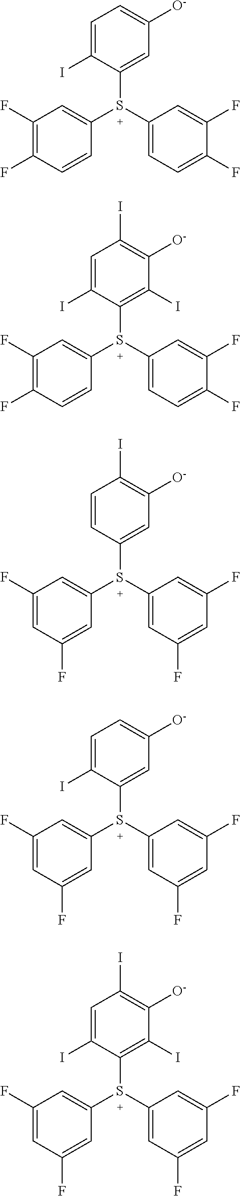

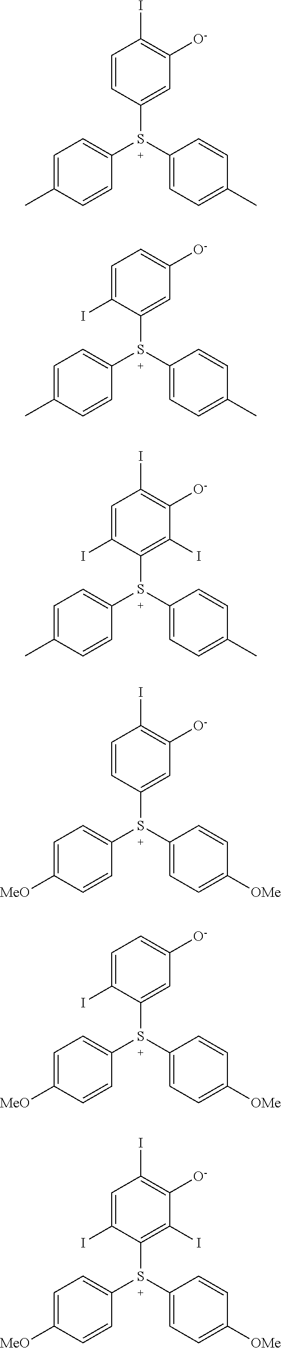

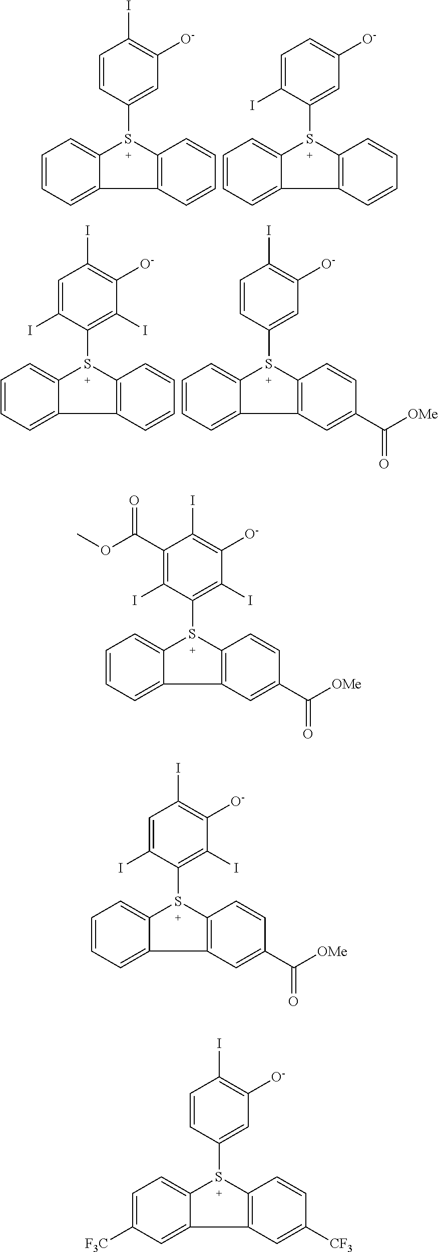

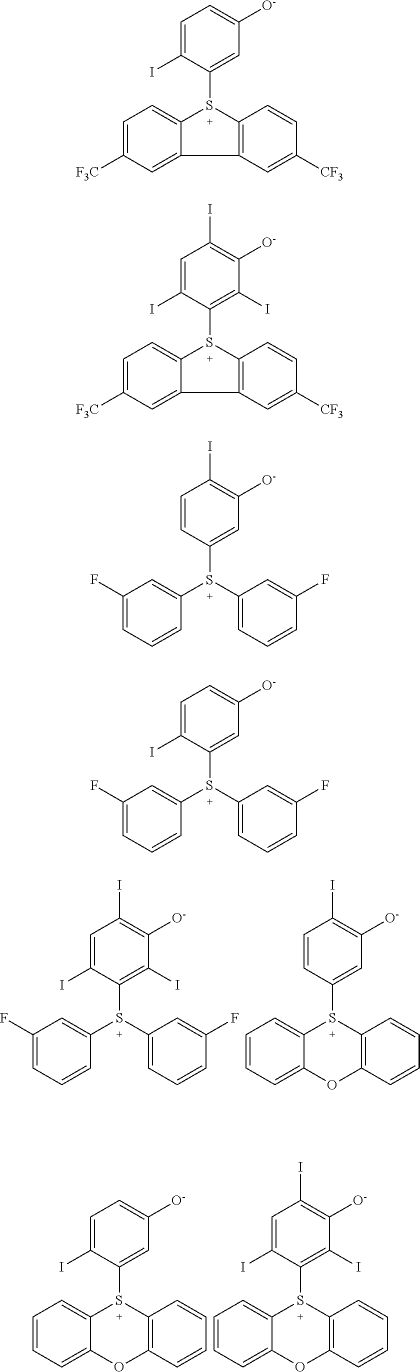

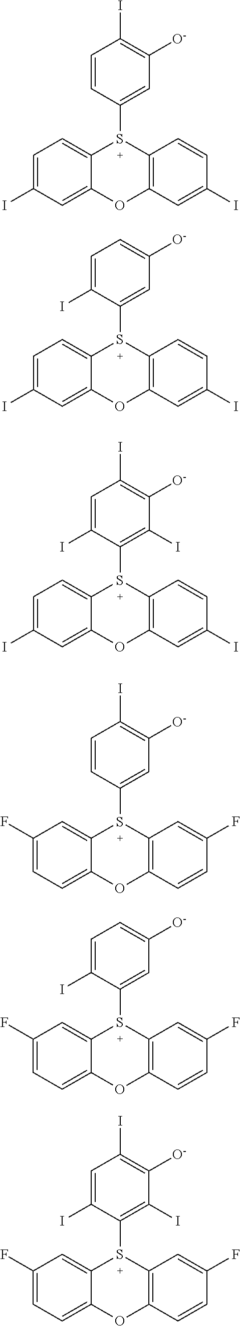

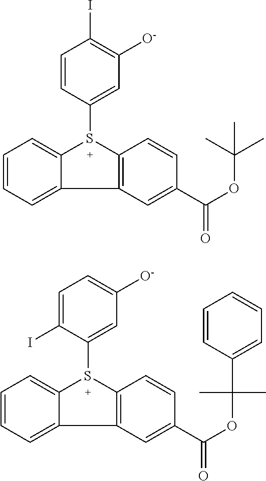

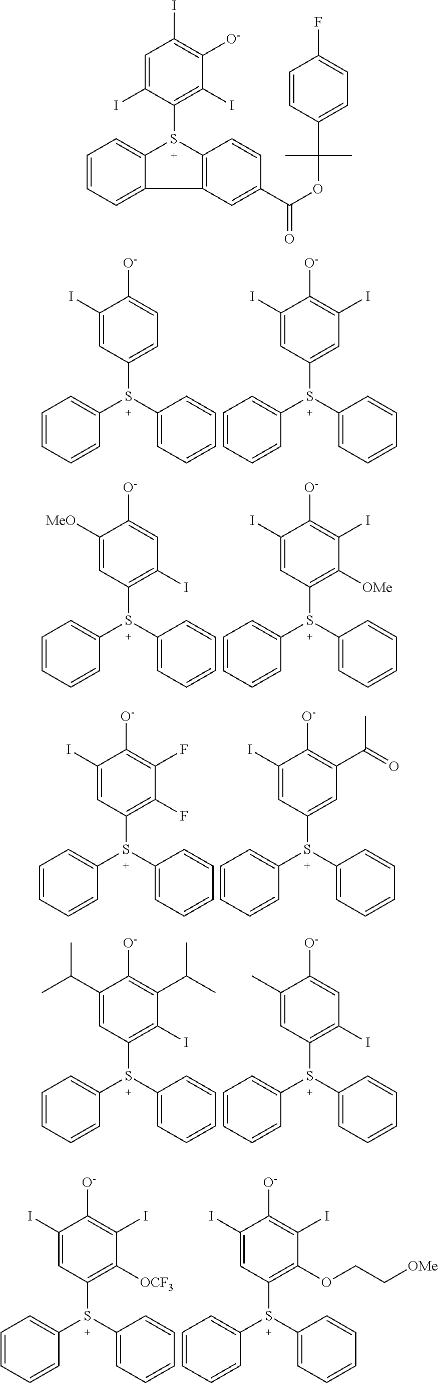

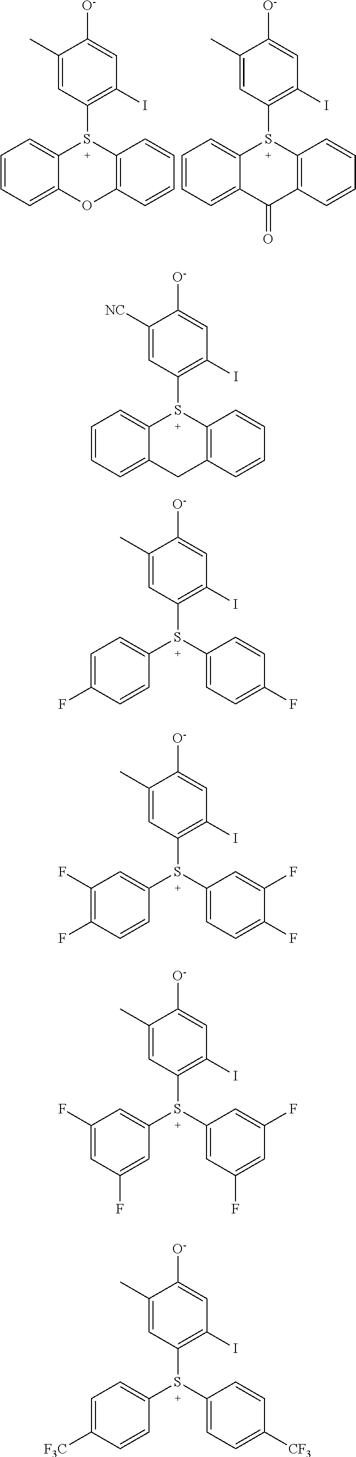

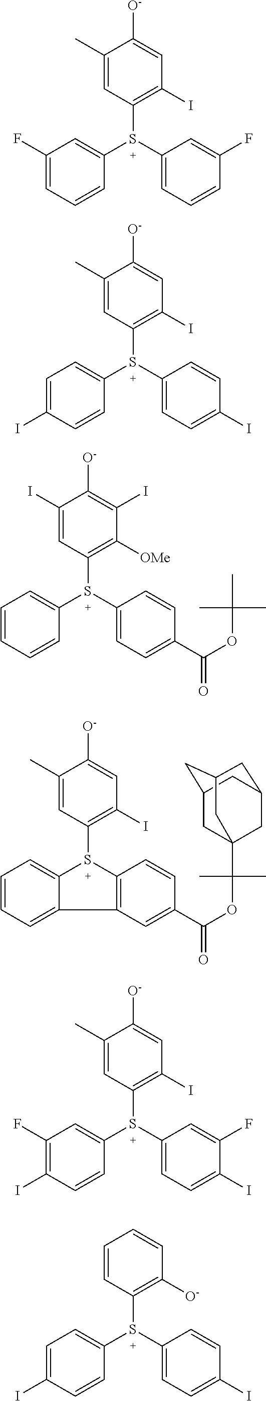

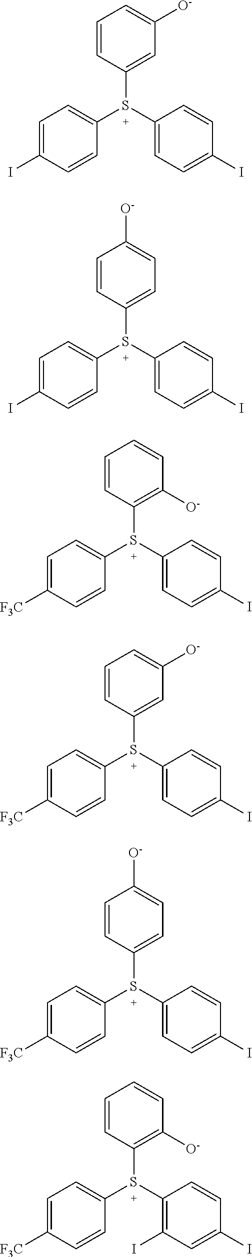

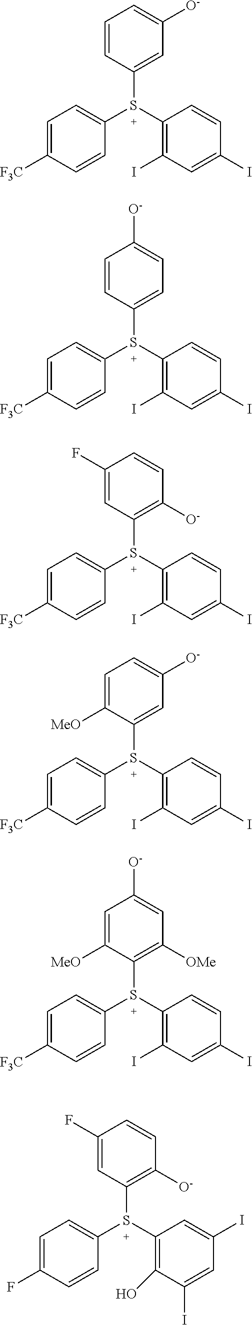

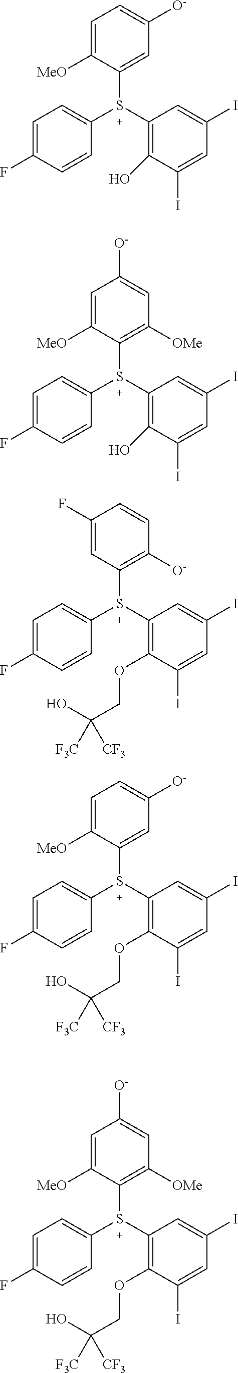

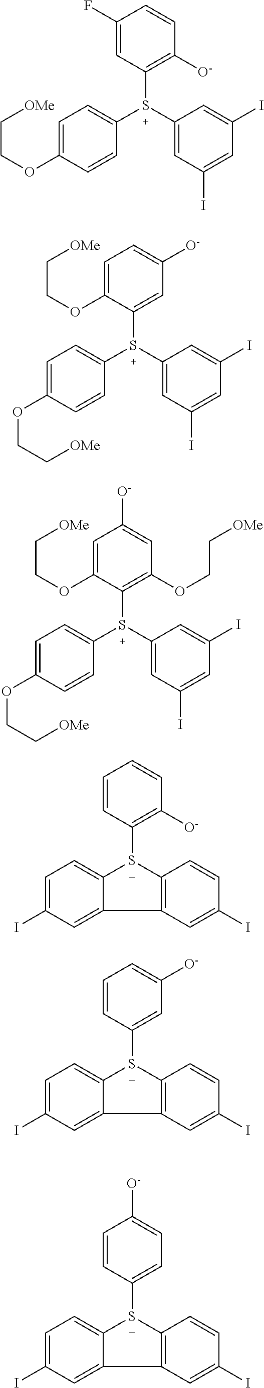

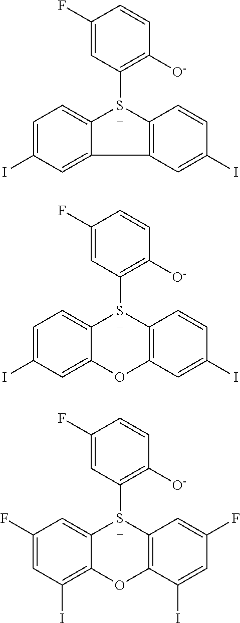

The inventive sulfonium salt can be synthesized, for example, by the following method, but is not limited thereto.

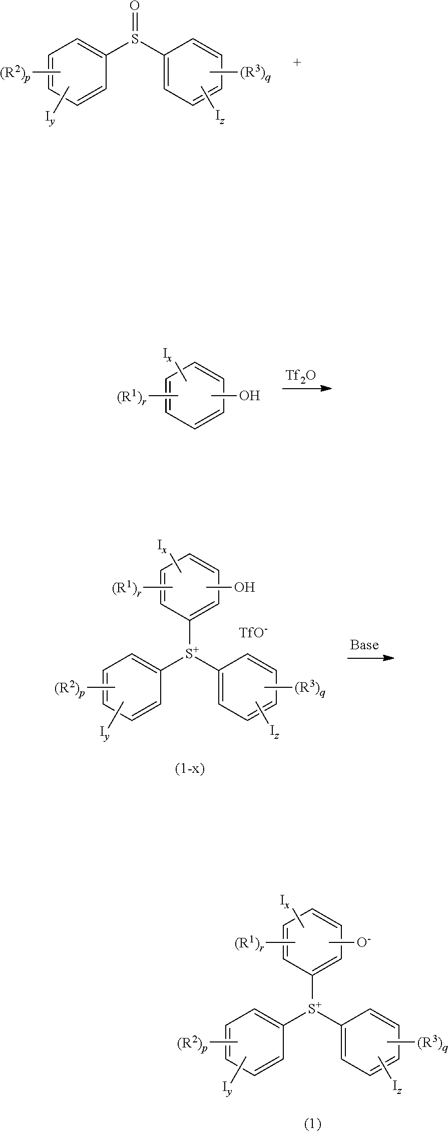

The corresponding sulfoxide and phenol are cationized in an organic solvent in the presence of a reaction activating agent such as trifluoromethanesulfonic anhydride or trifluoroacetic anhydride to obtain intermediate (1-x). The obtained sulfonium salt (1-x) is deprotonated under basic conditions to obtain the desired sulfonium salt (1).

The organic solvent used in the cationization of the first step may be, for example, methylene chloride, but is not particularly limited as long as it does not inhibit the reaction. Organic solvents having a benzene ring, such as benzene and toluene, are not suitable for the above synthesis example because they cause a side reaction with sulfoxide. In addition, acids such as trifluoromethanesulfonic acid and methanesulfonic acid may be added from the viewpoint of controlling reactivity.

The deprotonation of the second step can be easily carried out using an inorganic base such as sodium bicarbonate or an organic base such as pyridine and triethylamine. For example, the intermediate (1-x) is dissolved in a two-layer solvent of methylene chloride and pure water, and then an equimolar amount of the above-mentioned base is added thereto and stirred for about 30 minutes to 1 hour to obtain the desired sulfonium salt (1).

The inventive sulfonium salt may also be synthesized by the following method.

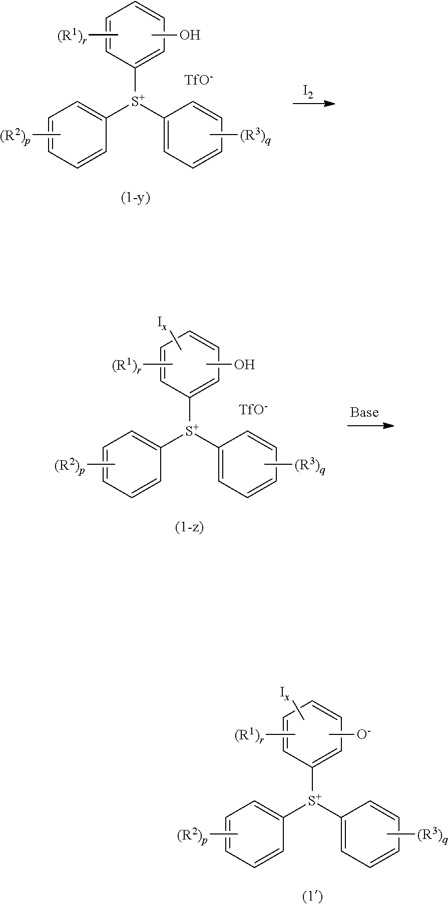

The phenol group-containing cationic intermediate (1-y) is iodized to synthesize an iodophenol-containing cationic intermediate (1-z), which is then subjected to basic conditions in the same manner as above to obtain the desired sulfonium salt (1′).

In the iodination of the first step, the reaction solvent is not particularly limited as long as it does not inhibit the reaction. For the iodinating agent, it is possible to use iodine, iodine chloride, N-iodosuccinimide, etc. In order to increase the reactivity, a basic compound may be added as necessary.

The application of the inventive sulfonium salt as an acid diffusion inhibitor to a resist material leads to improvements in various lithography performances such as sensitivity, LWR, and CDU. Although the details are unclear, this can be considered as follows.

The inventive sulfonium salt is characterized by having both an anion and a cation in one molecule. Usually, as the ratio of monomer components in the entire resist composition increases, the glass transition temperature (hereinafter, Tg) of the formed resist film decreases. This is considered to be because the monomer components, which have a higher degree of freedom compared to the polymer components, move and disperse easily due to heat. It is considered that: the ratio of monomer components added is reduced because the inventive sulfonium salt has an anionic moiety and a cationic moiety in the same molecule; thus the Tg of the resist film increases; and, as the result, acid diffusion is suppressed so that various lithography performances are improved.

The inventive sulfonium salt is also characterized by having an iodine atom as a substituent. Having iodine atoms enables efficient absorption of EUV light, leading to sensitivity enhancement. Furthermore, there is a risk that fat-soluble decomposition products such as sulfides produced by photolysis of cations may cause a residual defect during alkaline development due to the dissolution-inhibiting properties of iodine atoms, however, it is considered that a photodecomposition product having an iodine atom have alkaline affinity, making defects less likely to occur when an iodine atom and a phenoxy anion moiety are present on the same benzene ring.

### [Resist Composition]

Further, the present invention also provides a chemically-amplified resist composition that contains the inventive sulfonium salt as an acid diffusion inhibitor.

That is, the present invention provides a resist composition (chemically-amplified resist composition) comprising a resin component (component A) that changes its solubility in a developer by an action of an acid, a photo-acid generator (component B), an organic solvent (component D), and the acid diffusion inhibitor (component C-1) above.

Further, the present invention provides a resist composition (chemically-amplified resist composition) comprising a resin component (component A-1) that generates an acid by exposure to light and changes its solubility in a developer by an action of an acid, an organic solvent (component D), and the acid diffusion inhibitor (component C-1) above.

The acid diffusion inhibitor (component C-1) is the inventive sulfonium salt above. The amount of the acid diffusion inhibitor (component C-1) is not particularly limited, but may be, for example, 1 to 50 parts by mass, preferably 3 to 25 parts by mass, based on 100 parts by mass of the resin component (component A) or the resin component (component A-1) described later.

### [Polymer]

In the chemically-amplified resist composition, the polymer in the present invention is a resin component (component A) that changes its solubility in a developer by an action of an acid, or a resin component (component A-1) that functions as a photo-acid generator and as a base polymer.

The polymer in the present invention may contain a repeating unit represented by the following formula (b1) (hereinafter also referred to as the repeating unit b1) or a repeating unit represented by the following formula (b2) (hereinafter also referred to as the repeating unit b2).

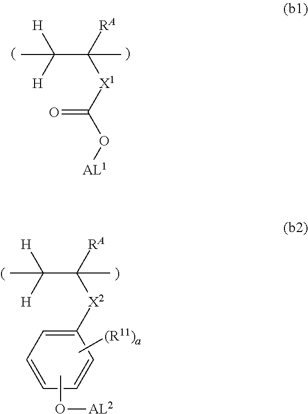

In the formulae (b1) and (b2), R^{A} each independently represents a hydrogen atom, a fluorine atom, a methyl group, or a trifluoromethyl group.

In the formula (b1), X¹ represents a single bond, a phenylene group, a naphthylene group, “*—C(═O)—O—X¹¹—”, or “*—C(═O)—NH—X¹¹—”, and the phenylene group or the naphthylene group may be substituted with a hydroxy group, a nitro group, a cyano group, a saturated hydrocarbyl group having 1 to 10 carbon atoms and optionally having a fluorine atom, an alkoxy group having 1 to 10 carbon atoms and optionally having a fluorine atom, or a halogen atom. X¹¹ is a saturated hydrocarbylene group having 1 to 10 carbon atoms, a phenylene group, or a naphthylene group, and the saturated hydrocarbylene group may have a hydroxy group, an ether bond, an ester bond, or a lactone ring. “*” represents a bond to a carbon atom of the main chain.

In the formula (b2), X² is a single bond, “*—C(═O)—O—”, or “*—C(═O)—NH—”. “*” represents a bond to a carbon atom in the main chain. R¹¹ represents a halogen atom, a cyano group, a hydroxy group, a nitro group, a hydrocarbyl group having 1 to 20 carbon atoms and optionally having a heteroatom, a hydrocarbyloxy group having 1 to 20 carbon atoms and optionally having a heteroatom, a hydrocarbylcarbonyl group having 2 to 20 carbon atoms and optionally having a heteroatom, a hydrocarbylcarbonyloxy group having 2 to 20 carbon atoms and optionally having a heteroatom, or a hydrocarbyloxycarbonyl group having 2 to 20 carbon atoms and optionally having a heteroatom. “a” is an integer of 0 to 4, preferably 0 or 1.

In the formulae (b1) and (b2), AL¹ and AL² are each independently an acid-labile group. Specific examples of the acid-labile group include those described in JP2013-80033A and JP2013-83821A, but are not limited thereto.

Typically, specific examples of the acid-labile group include those represented by the following formulae (AL-1) to (AL-3).

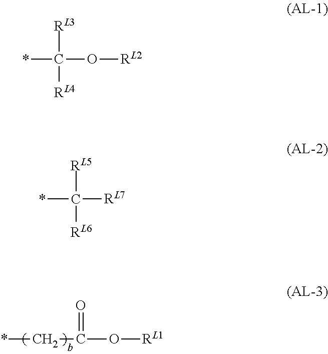

In the formulae, “*” represents a bond.

In the formula (AL-1), R^{L2} is a hydrocarbyl group having 1 to 40 carbon atoms, and may contain a heteroatom such as an oxygen atom, a sulfur atom, a nitrogen atom, a fluorine atom, and an iodine atom. The hydrocarbyl group may be saturated or unsaturated, and may be linear, branched, or cyclic. The hydrocarbyl group preferably has 1 to 20 carbon atoms.

In the formula (AL-1), R^{L3} and R^{L4} are each independently a hydrogen atom or a hydrocarbyl group having 1 to 20 carbon atoms, and may contain a heteroatom such as an oxygen atom, a sulfur atom, a nitrogen atom, a fluorine atom, or an iodine atom. The hydrocarbyl group may be saturated or unsaturated, and may be linear, branched, or cyclic. Any two of R^{L2}, R^{L3}, and R^{L4} may be bonded to each other to form a ring having 3 to 20 carbon atoms together with the carbon atom to which they are bonded, or together with the carbon atom and the oxygen atom. The ring is preferably a ring having 4 to 16 carbon atoms, and particularly preferably an alicyclic ring.

In the formula (AL-2), R^{L5}, R^{L6}, and R^{L7} are each independently a hydrocarbyl group having 1 to 20 carbon atoms, and may contain a heteroatom such as an oxygen atom, a sulfur atom, a nitrogen atom, a fluorine atom, and an iodine atom. The hydrocarbyl group may be saturated or unsaturated, and may be linear, branched, or cyclic. Any two of R^{L5}, R^{L6}, and R^{L7} may be bonded to each other to form a ring having 3 to 20 carbon atoms together with the carbon atom to which they are bonded. The ring is preferably a ring having 4 to 16 carbon atoms, and particularly preferably an alicyclic ring.

In the formula (AL-3), “b” represents an integer of 0 to 10, and preferably an integer of 1 to 5.

In the formula (AL-3), R^{L1} has a structure represented by a partial structural formula (AL-1) or (AL-2).

Specific examples of the repeating unit b1 include those shown below, but are not limited thereto. In the following formulae, R^{A} and AL¹ are the same as defined above.

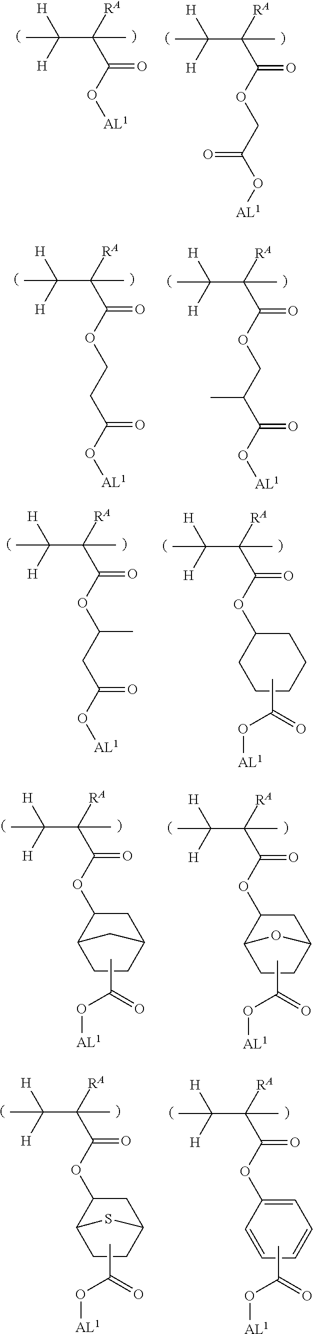

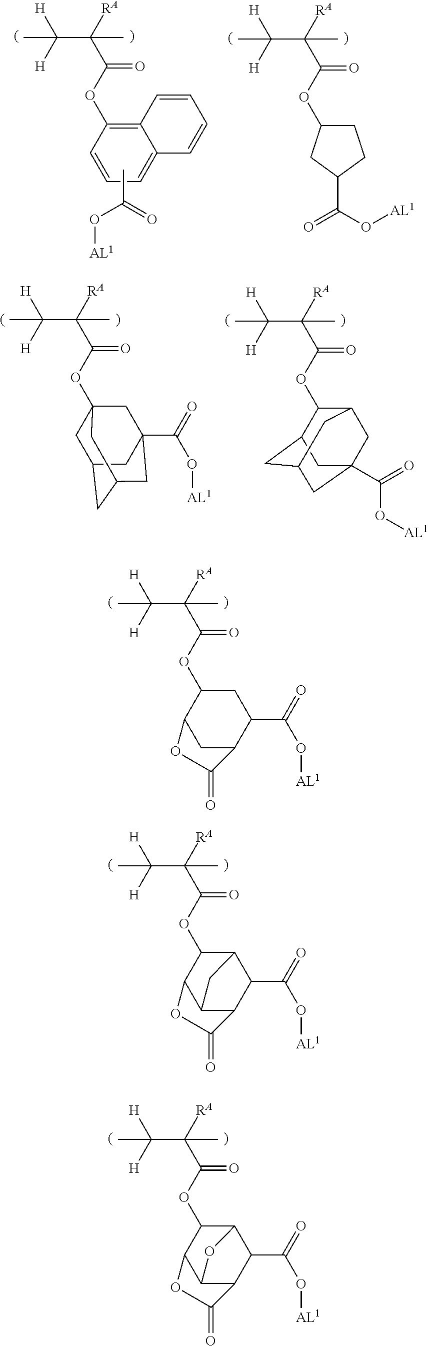

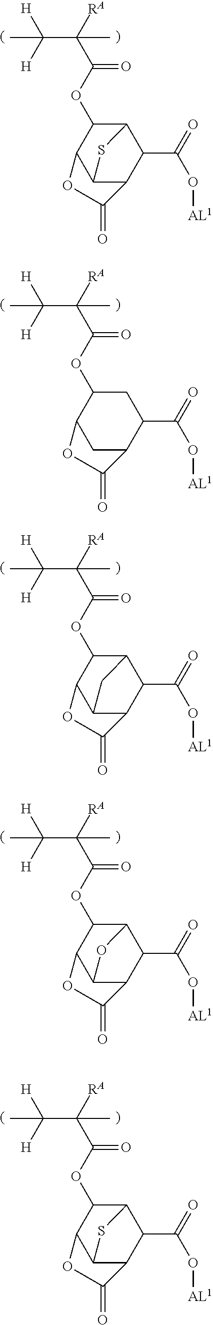

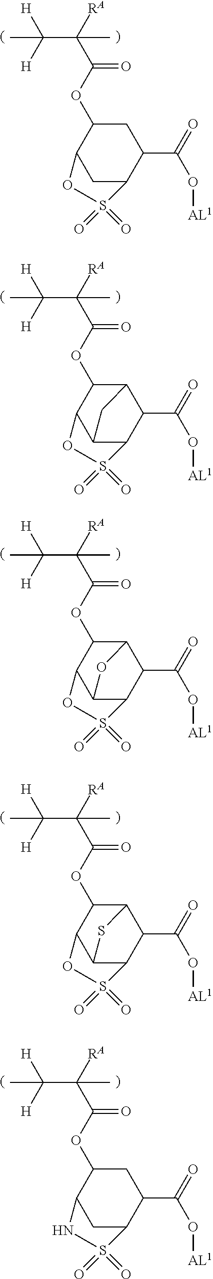

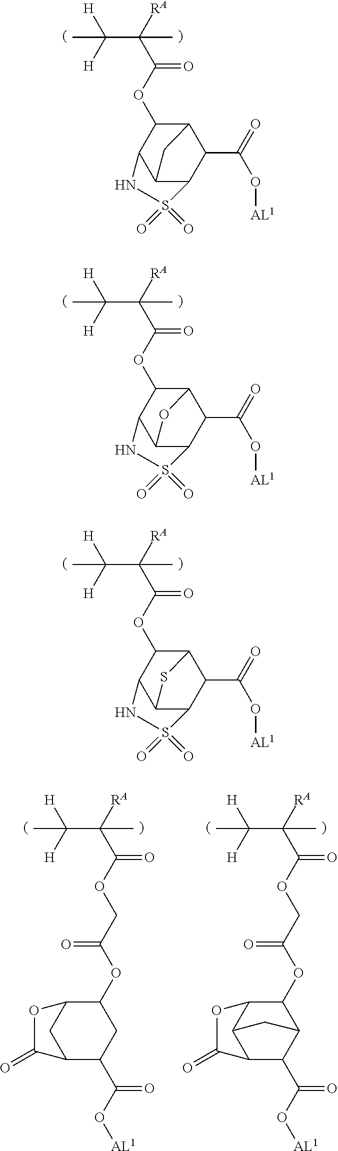

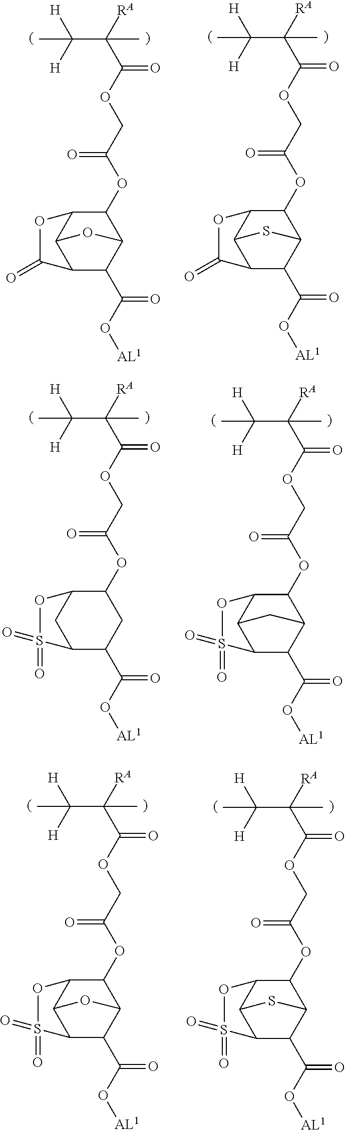

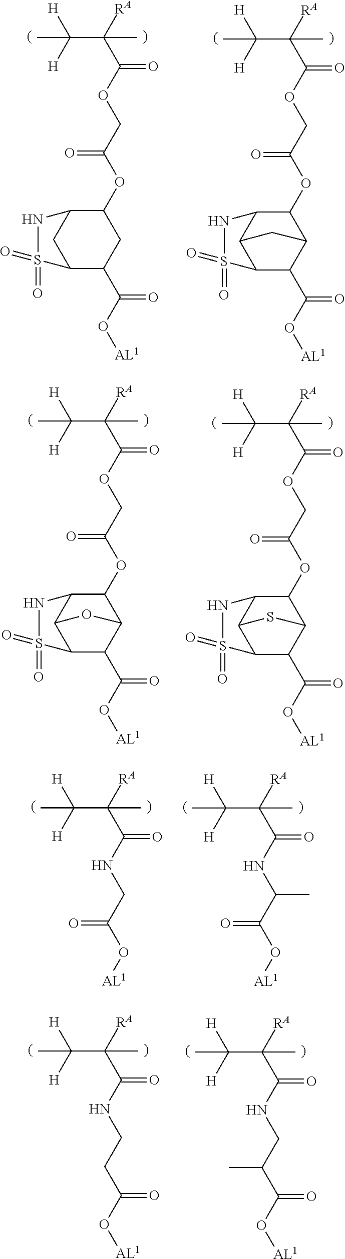

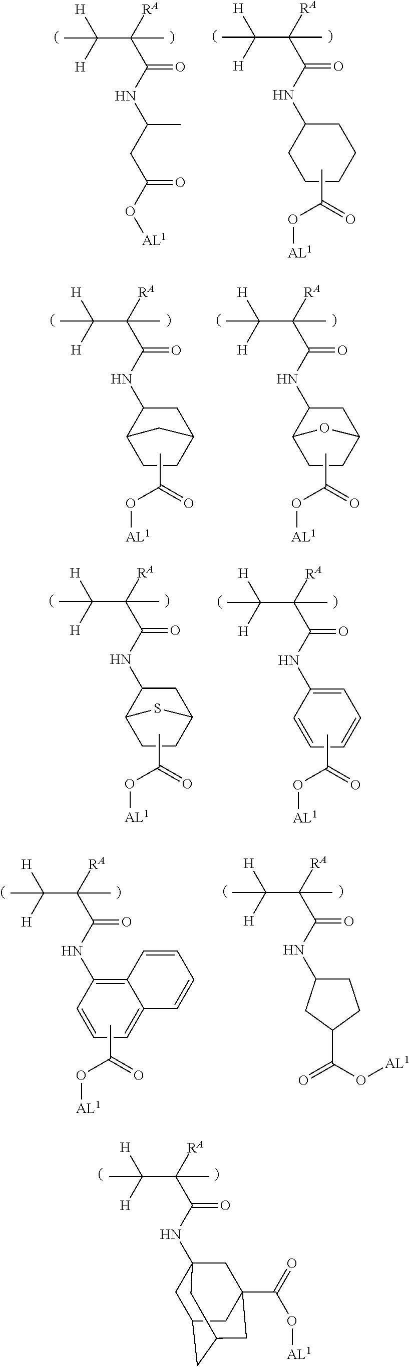

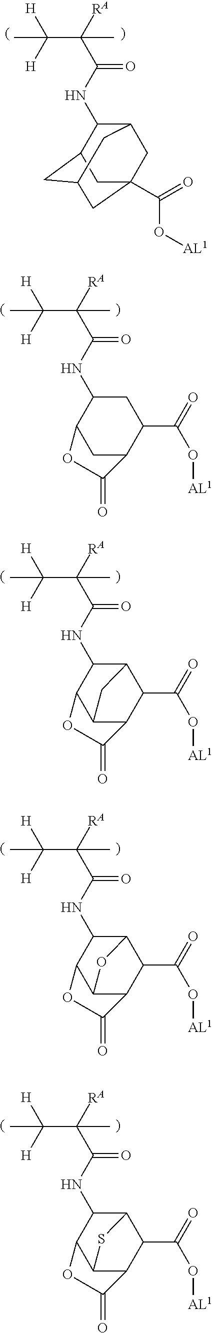

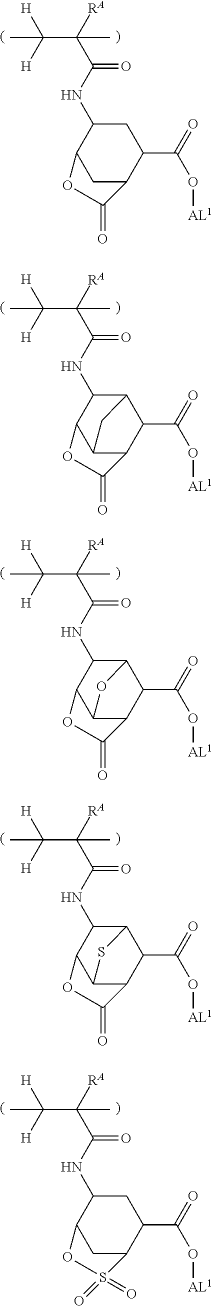

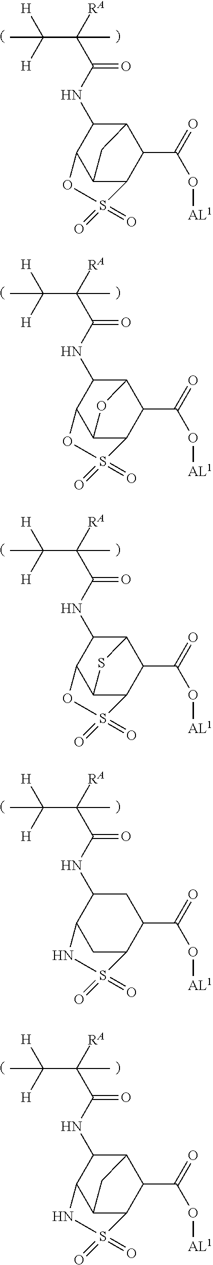

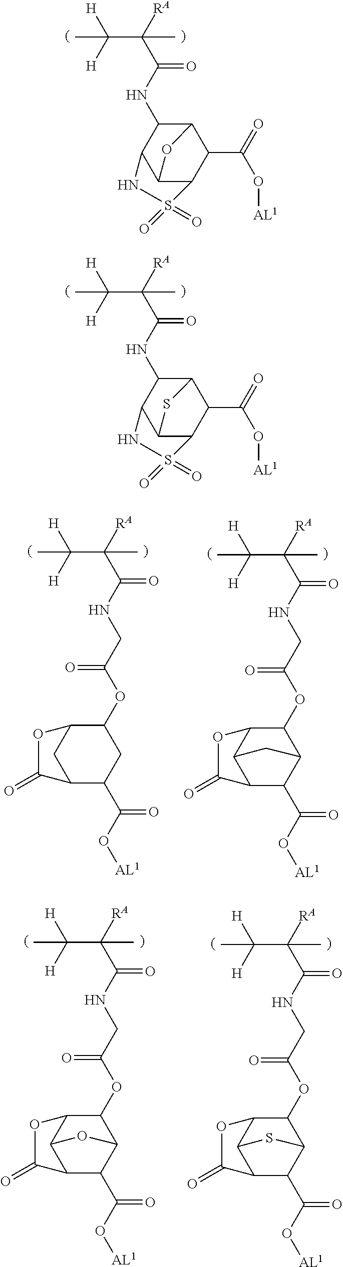

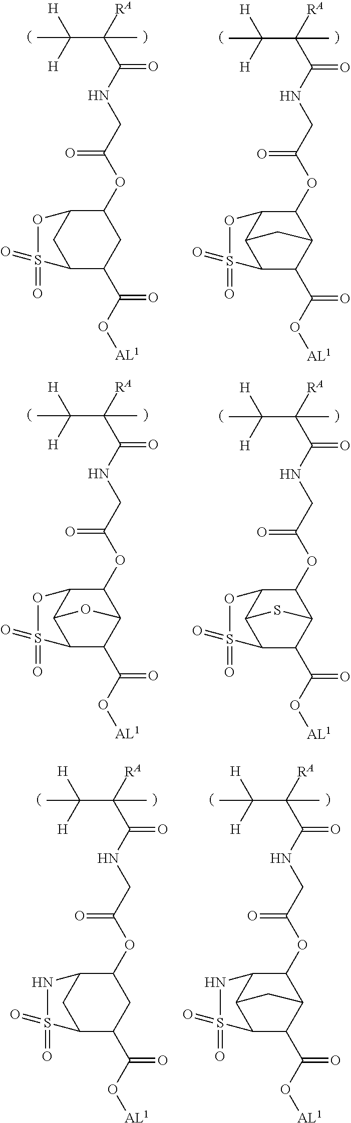

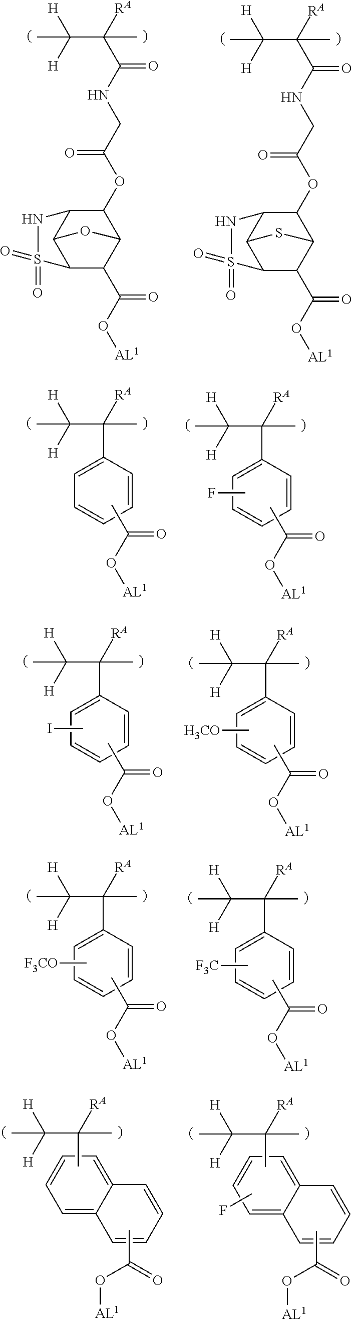

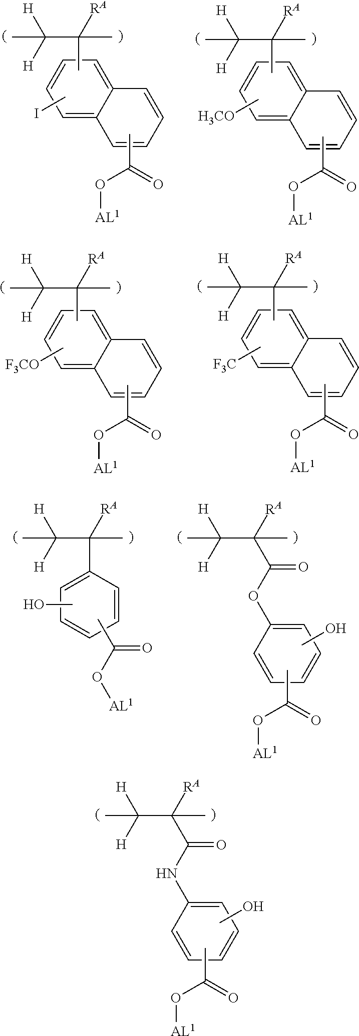

Specific examples of the repeating unit b2 include those shown below, but are not limited thereto. In the following formulae, R^{A} and AL² are the same as defined above.

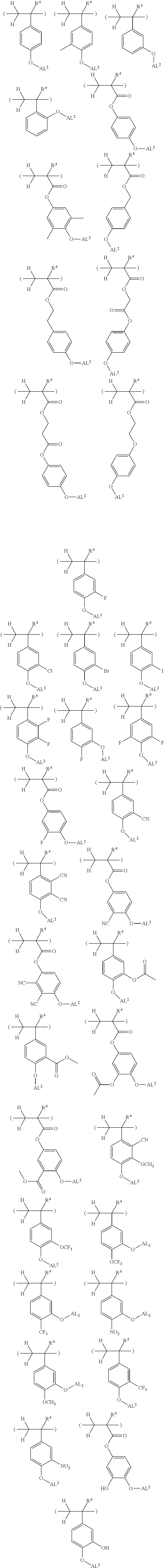

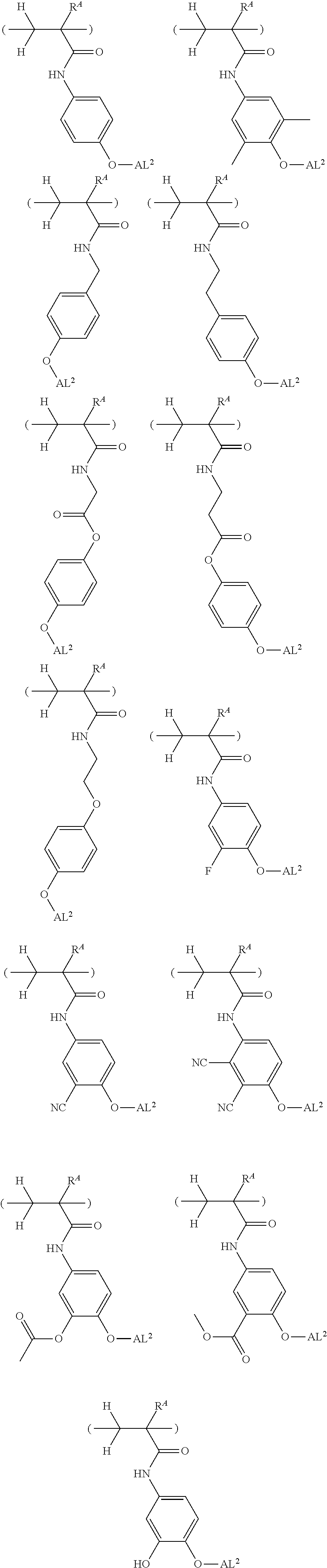

The repeating units b1 and b2 are particularly preferably the repeating units exemplified below.

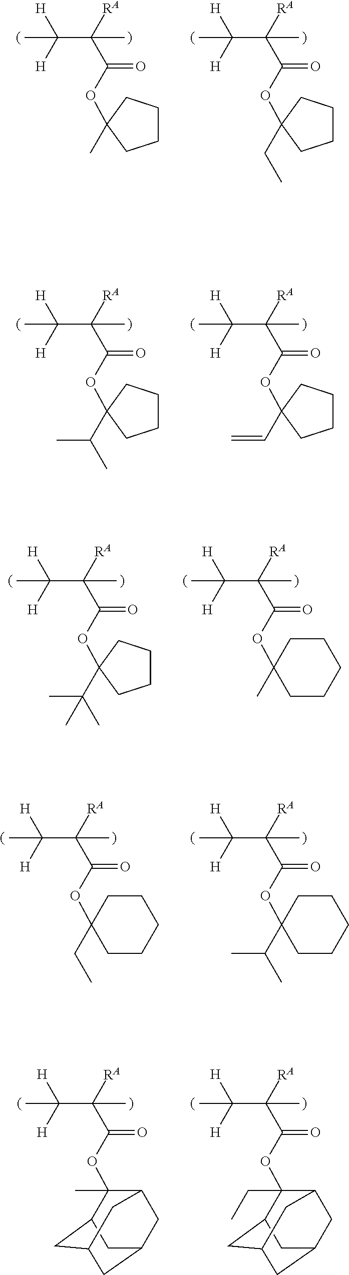

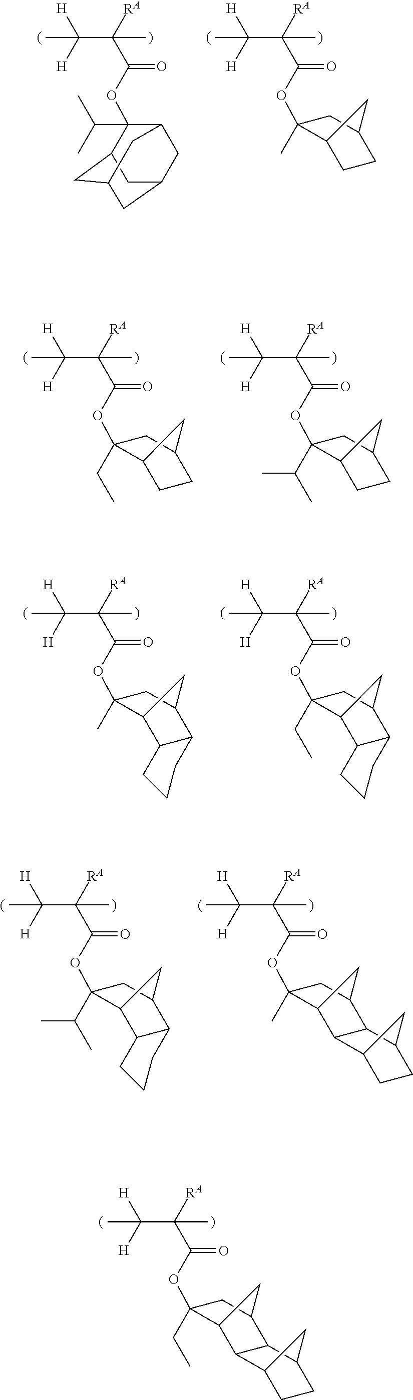

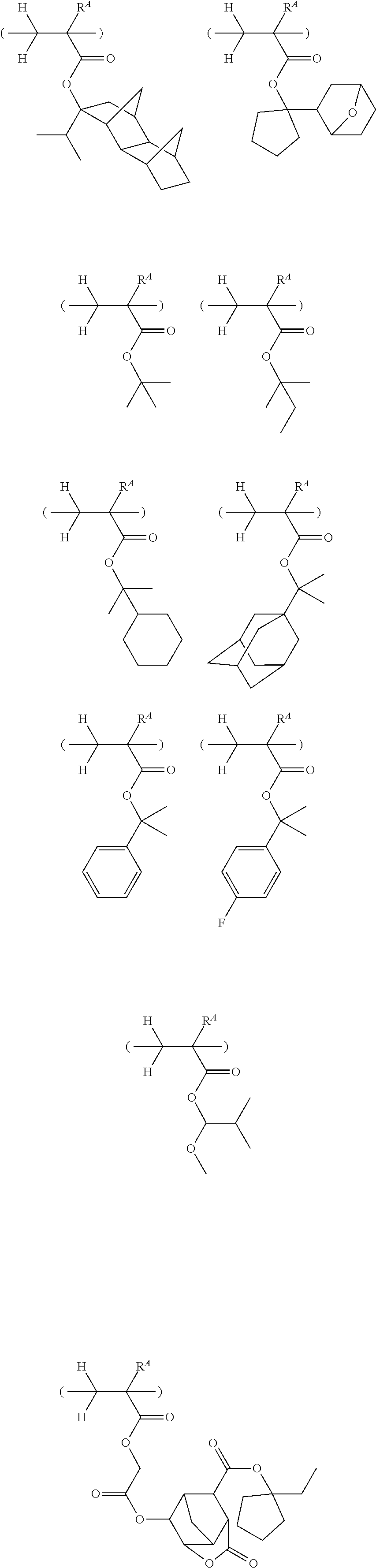

The polymer may further have a repeating unit represented by the following formula (b3) (hereinafter also referred to as the repeating unit b3).

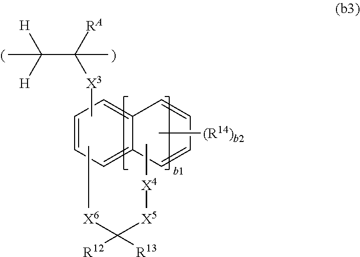

In the formula (b3), b1 is 0 or 1. When b1 is 0, it is a benzene ring, and when b1 is 1, it is a naphthalene ring. However, from the viewpoint of solvent solubility, b1 is preferably 0, resulting in a benzene ring. When b1 is 0, b2 is an integer of 0 to 3, and when b1 is 1, b2 is an integer of 0 to 5. From the viewpoint of raw material procurement, b2 is preferably 0, 1, 2 or 3, and more preferably 0, 1 or 2.

In the formula (b3), R^{A} is a hydrogen atom, a fluorine atom, a methyl group, or a trifluoromethyl group. Among these, it is preferably a hydrogen atom or a methyl group, and more preferably a hydrogen atom.

In the formula (b3), X³ is a single bond, “*—C(═O)—O—”, or “*—C(═O)—NH—”. “*” represents a bond to a carbon atom of the main chain. Among these, it is preferably a single bond or “*—C(═O)—”, and further preferably a single bond.

In the formula (b3), R¹² and R¹³ each independently represent a hydrogen atom or a hydrocarbyl group having 1 to 20 carbon atoms and optionally having a heteroatom. The hydrocarbyl group may be saturated or unsaturated, and may be linear, branched, or cyclic. Examples thereof include: alkyl groups having 1 to 20 carbon atoms, such as a methyl group, an ethyl group, an n-propyl group, an isopropyl group, an n-butyl group, an isobutyl group, an sec-butyl group, a tert-butyl group, an n-pentyl group, an n-hexyl group, an n-octyl group, an n-nonyl group, an n-decyl group, a undecyl group, a dodecyl group, a tridecyl group, a tetradecyl group, a pentadecyl group, a heptadecyl group, a octadecyl group, a nonadecyl group, and an icosyl group; cyclic saturated hydrocarbyl groups having 3 to 20 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 20 carbon atoms, such as a vinyl group, an allyl group, a propenyl group, a butenyl group, and a hexenyl group; cyclic unsaturated hydrocarbyl groups having 3 to 20 carbon atoms, such as a cyclohexenyl group; aryl groups having 2 to 20 carbon atoms such as a phenyl group and a naphthyl group; aralkyl groups having 7 to 20 carbon atoms, such as a benzyl group, a 1-phenylethyl group, and a 2-phenylethyl group; and group obtained by combining these groups. In addition, a part or all of the hydrogen atoms of the hydrocarbyl group may be substituted with a group having a heteroatom such as an oxygen atom, a sulfur atom, a nitrogen atom, and a halogen atom, and some of the “—CH₂—” of the hydrocarbyl group may be substituted with a group having a heteroatom such as an oxygen atom, a sulfur atom, and a nitrogen atom. As a result, the hydrocarbyl group may have a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic acid anhydride (—C(═O)—O—C(═O)—), a haloalkyl group, etc.

In addition, R¹² and R¹³ may be bonded to each other to form a ring together with the carbon atom to which they are bonded. Specific examples of the ring formed in this case include a cyclopropane ring, a cyclobutane ring, a cyclopentane ring, a cyclohexane ring, a norbornane ring, and an adamantane ring. In addition, a part or all of the hydrogen atoms of the ring may be substituted with a group having a heteroatom such as an oxygen atom, a sulfur atom, a nitrogen atom, and a halogen atom, and some of the “—CH₂—” of the ring may be substituted with a group having a heteroatom such as an oxygen atom, a sulfur atom, and a nitrogen atom. As a result, the ring may have a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, sultone ring, carboxylic anhydride (—C(═O)—O—C(═O)—), a haloalkyl group, etc.

In the formula (b3), R¹⁴ represents a halogen atom, a hydroxy group, a cyano group, a nitro group, a hydrocarbyl group having 1 to 20 carbon atoms and optionally having a heteroatom, a hydrocarbyloxy group having 1 to 20 carbon atoms and optionally having a heteroatom, a hydrocarbyloxycarbonyl group having 2 to 20 carbon atoms and optionally having a heteroatom, a hydrocarbylthio group having 1 to 20 carbon atoms and optionally having a heteroatom, or “—N(R^{14A}) (R^{14B})”. R^{14A}and R^{14B} each independently represent a hydrogen atom or a hydrocarbyl group having 1 to 6 carbon atoms. The halogen atom is preferably a fluorine atom, a chlorine atom, a bromine atom, or an iodine atom, and more preferably a fluorine atom or an iodine atom. The hydrocarbyl group and the hydrocarbyl moiety of the hydrocarbyloxy groups, the hydrocarbyloxycarbonyl groups and the hydrocarbylthio groups may be saturated or unsaturated and may be linear, branched, or cyclic. Specific examples thereof include the same as those exemplified as the hydrocarbyl groups represented by R¹² and R¹³. In addition, a part or all of the hydrogen atoms of the hydrocarbyl group may be substituted with a group having a heteroatom such as an oxygen atom, a sulfur atom, a nitrogen atom, and a halogen atom, and some of the “—CH₂—” of the hydrocarbyl group may be substituted with a group having a heteroatom such as an oxygen atom, a sulfur atom, and a nitrogen atom. As a result, the hydrocarbyl group may have a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic acid anhydride (—C(═O)—O—C(═O)—), a haloalkyl group, etc. When b2 is 2 or more, R¹⁴s each may be the same or different from each other.

When b2 is 2 or more, R¹⁴s may be bonded to each other to form a ring together with the carbon atom of the aromatic ring to which they are bonded. Specific examples of the ring formed in this case include a cyclopropane ring, a cyclobutane ring, a cyclopentane ring, a cyclohexane ring, a norbornane ring, and an adamantane ring. In addition, a part or all of the hydrogen atoms of the ring may be substituted with a group having a heteroatom such as an oxygen atom, a sulfur atom, a nitrogen atom, and a halogen atom, and some of the “—CH₂—” of the ring may be substituted with a group having a heteroatom such as an oxygen atom, a sulfur atom, and a nitrogen atom. As a result, the ring may have a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic acid anhydride (—C(═O)—O—C(═O)—), a haloalkyl group, etc.

In the formula (b3), X⁴ represents a single bond, an aliphatic hydrocarbylene group having 1 to 4 carbon atoms, a carbonyl group, a sulfonyl group, or a group obtained by combining these groups. Among these, from the viewpoint of material procurement, a single bond, a carbonyl group, or a sulfonyl group is preferable, and from the viewpoint of a polar group generated after the reaction, a single bond or a carbonyl group is more preferable.

In the formula (b3), X⁵ and X⁶ represent each independently an oxygen atom or a sulfur atom. However, X⁴ and X⁶ are each bonded to a carbon atom adjacent to each other in the aromatic ring. X⁵ and X⁶ may be the same or different from each other, but from the viewpoint of reactivity, both X⁵ and X⁶ are preferably oxygen atoms.

Specific examples of the repeating unit b3 include those shown below, but are not limited thereto. In the following formula, R^{A} is the same as defined above, and Me represents a methyl group. In addition, the bonding positions of various substituents on an aromatic ring may be interchanged.

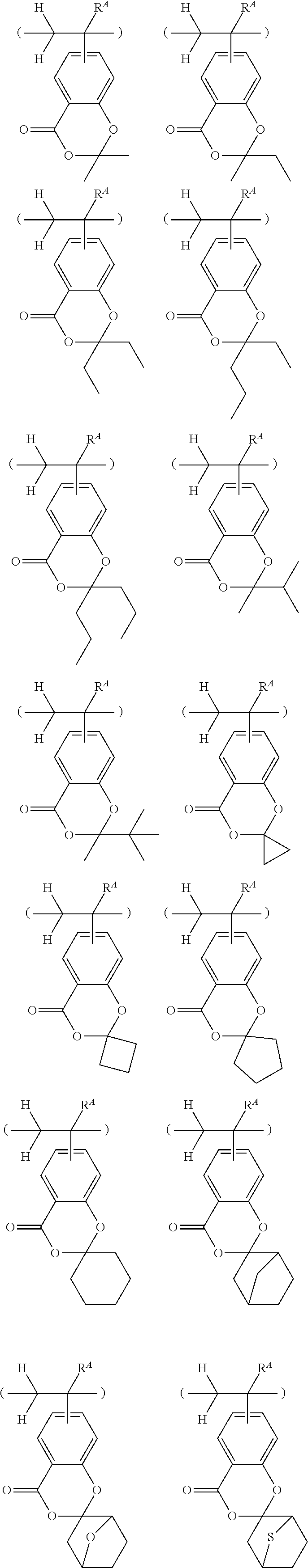

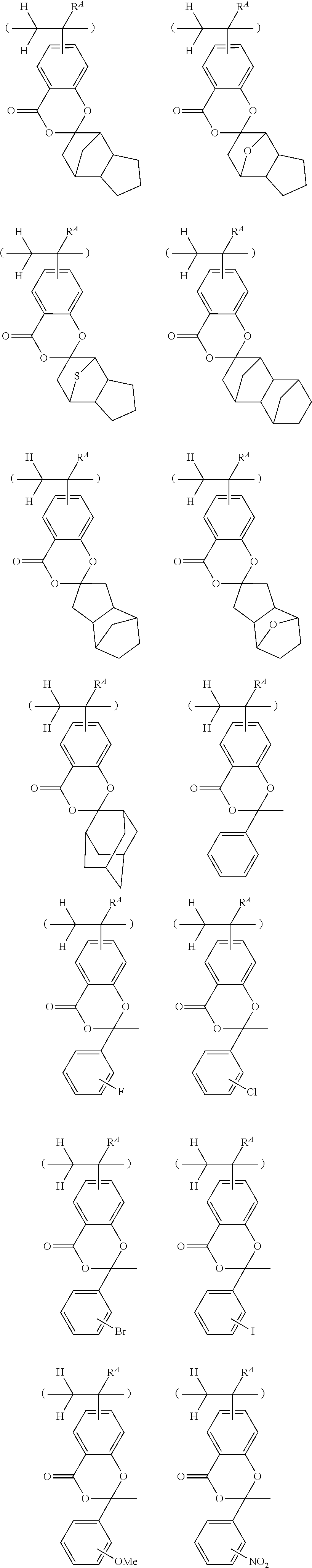

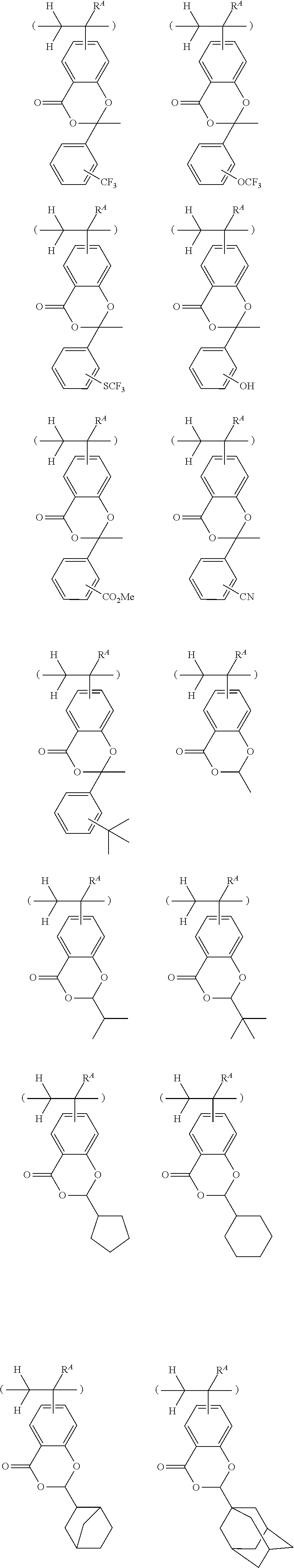

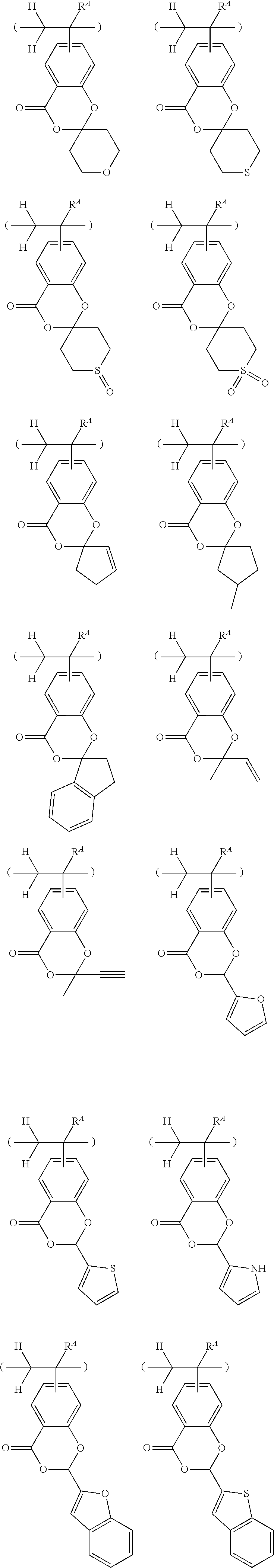

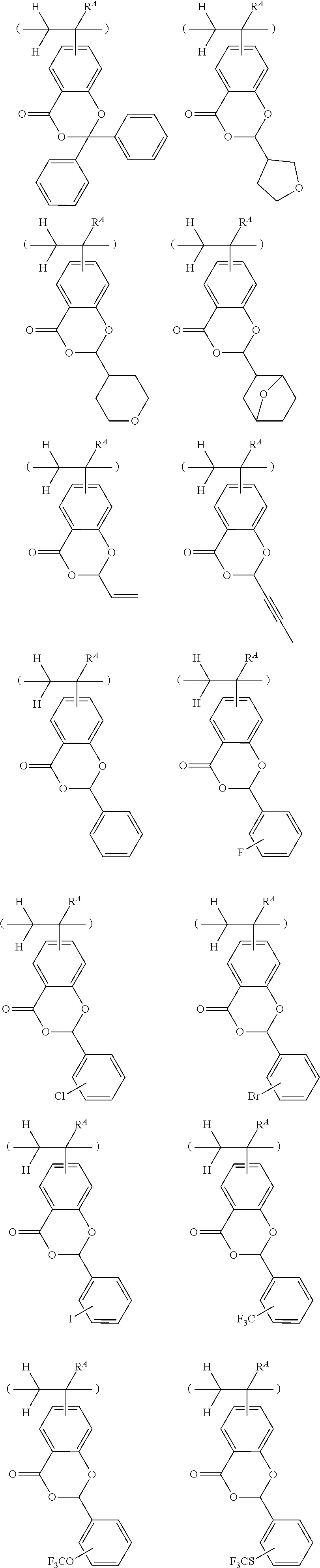

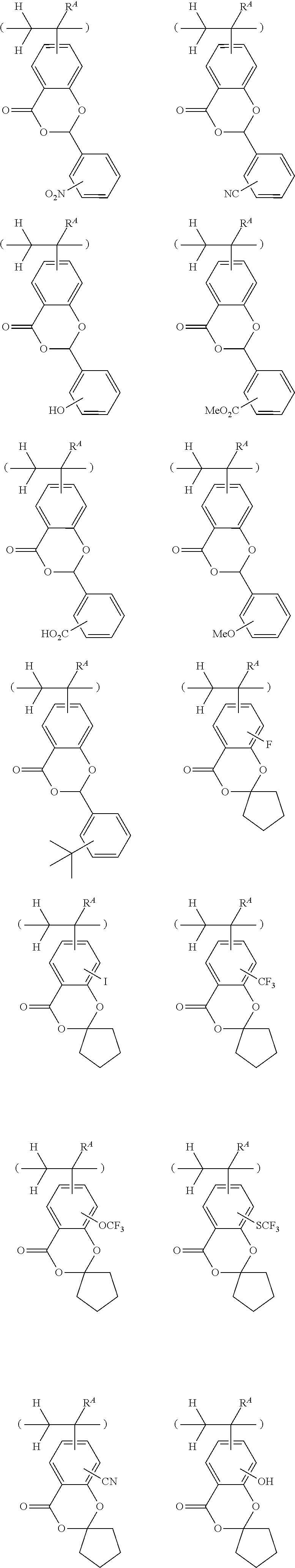

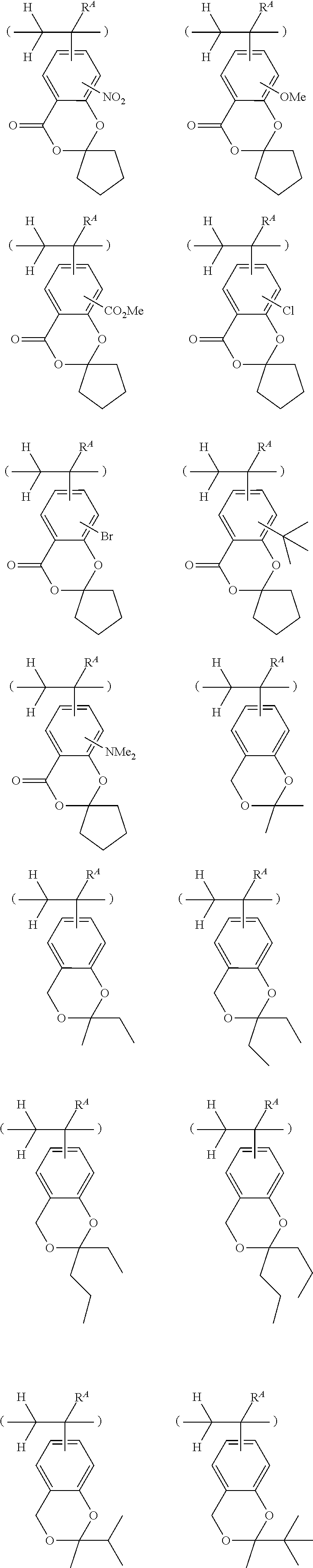

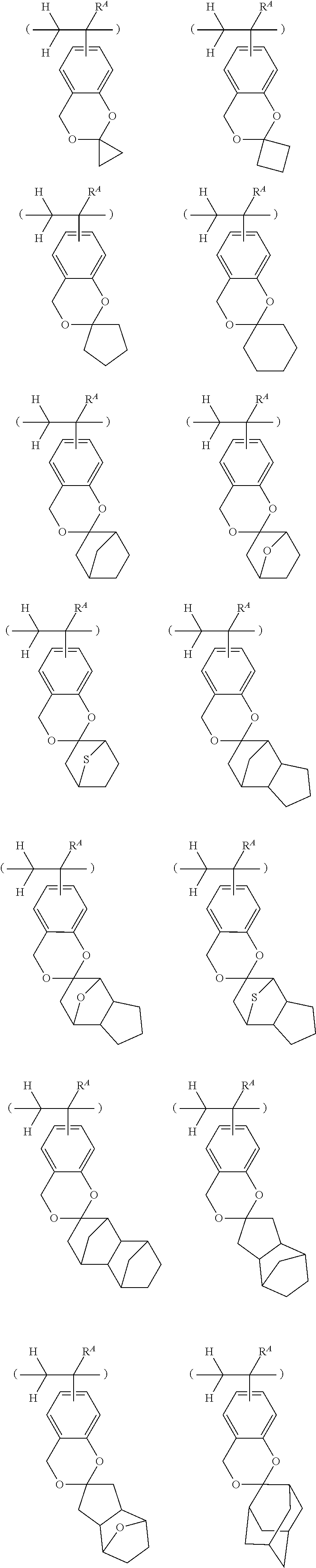

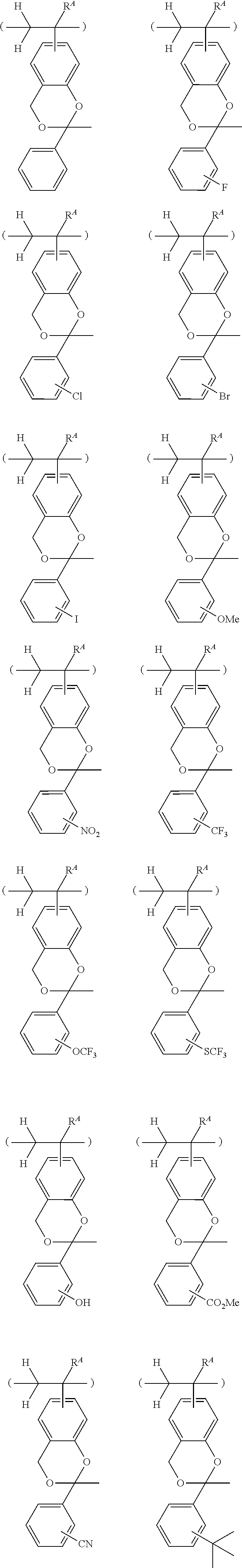

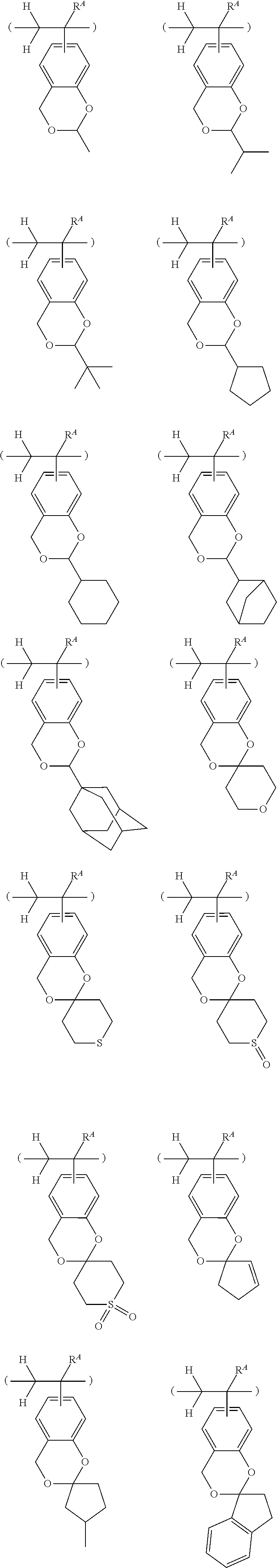

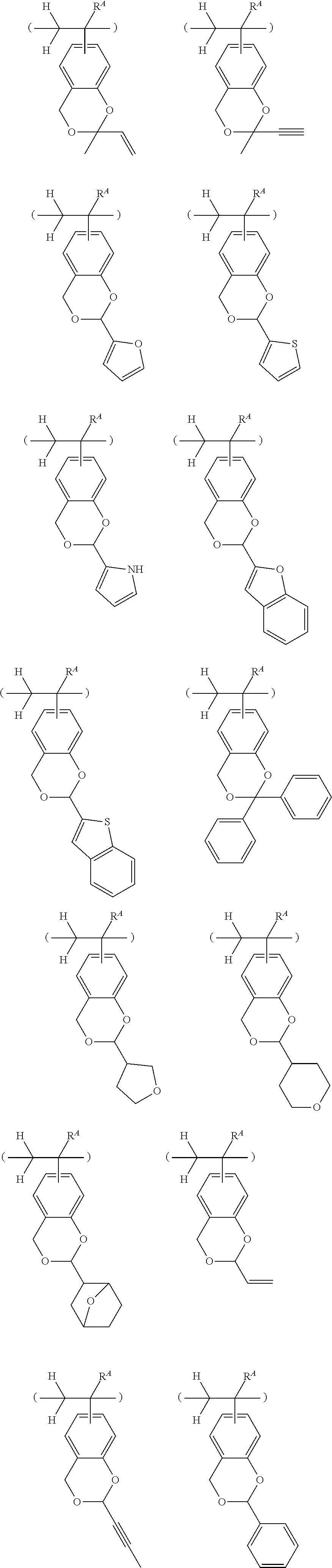

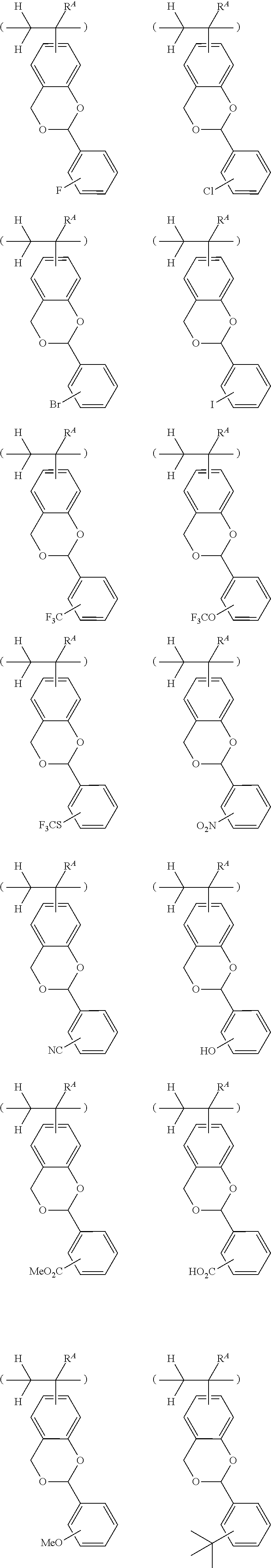

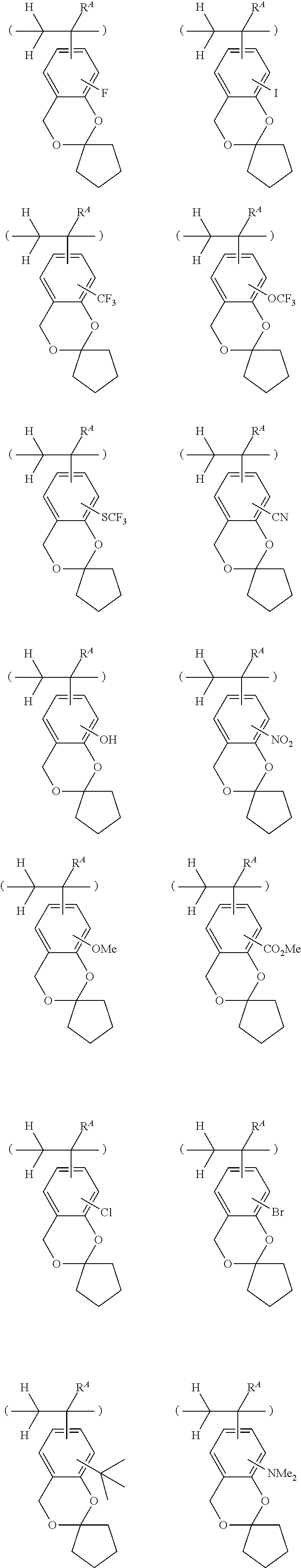

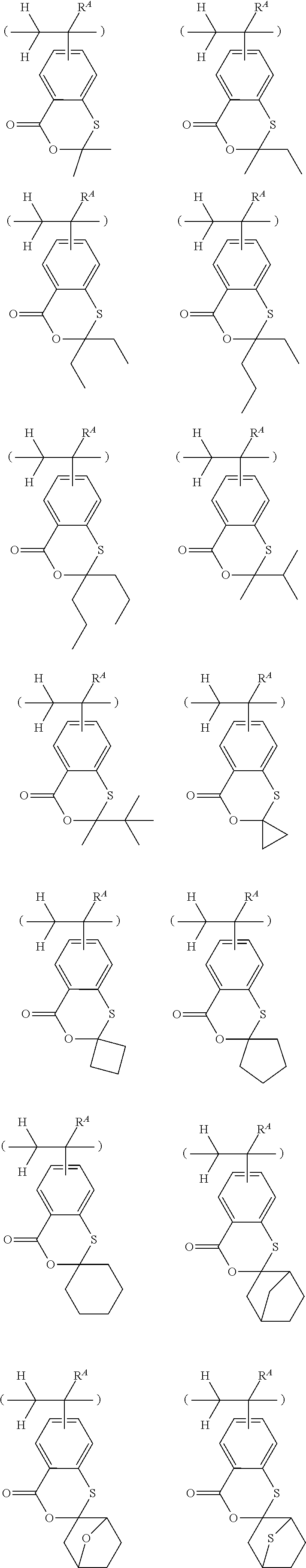

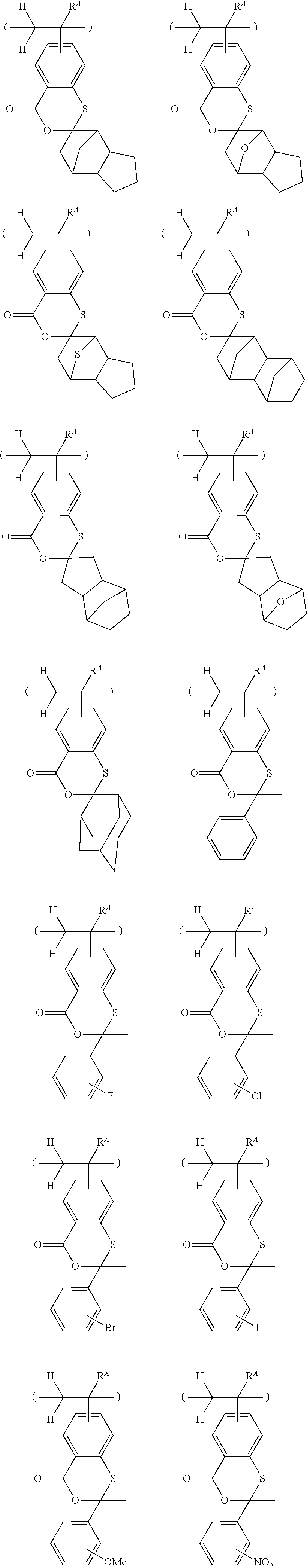

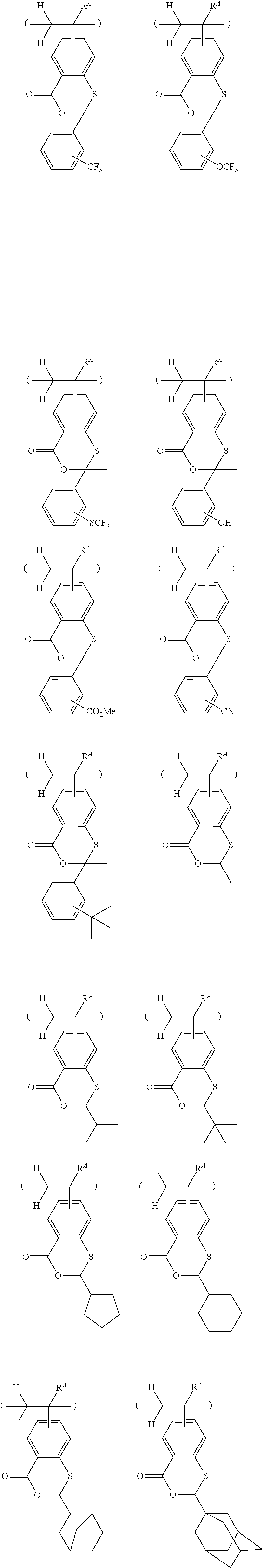

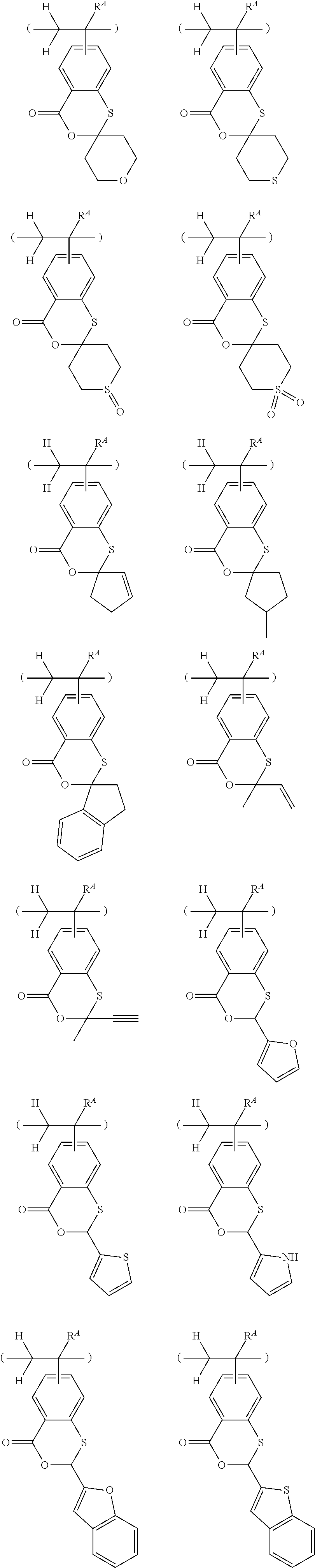

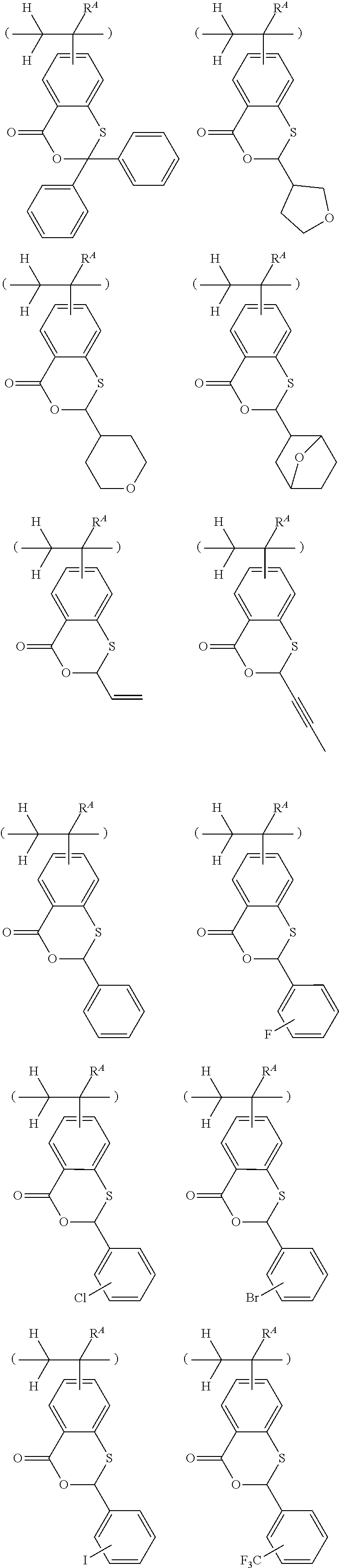

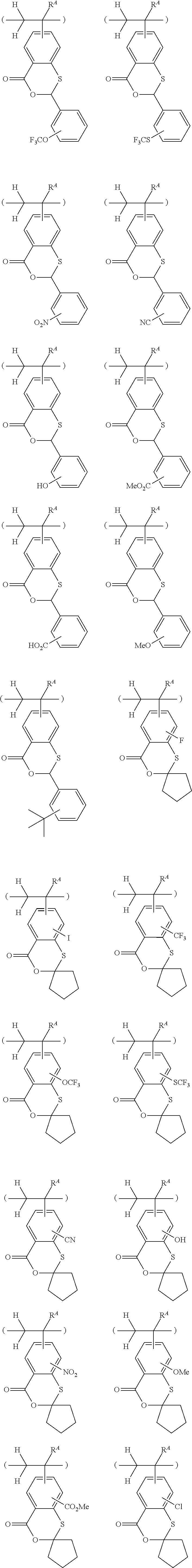

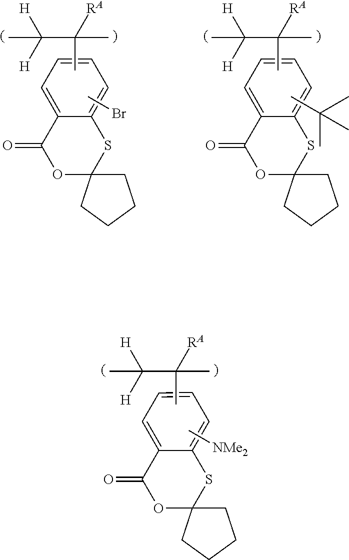

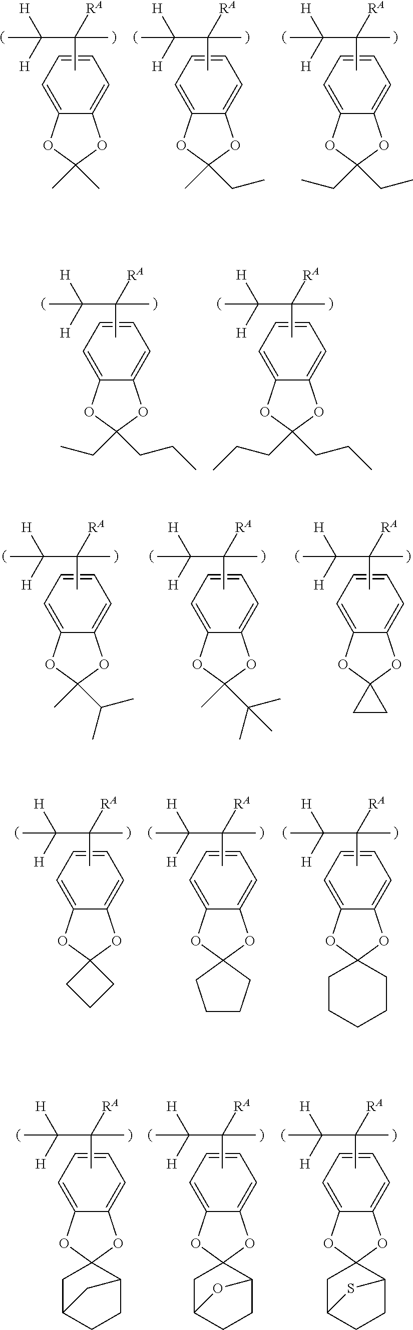

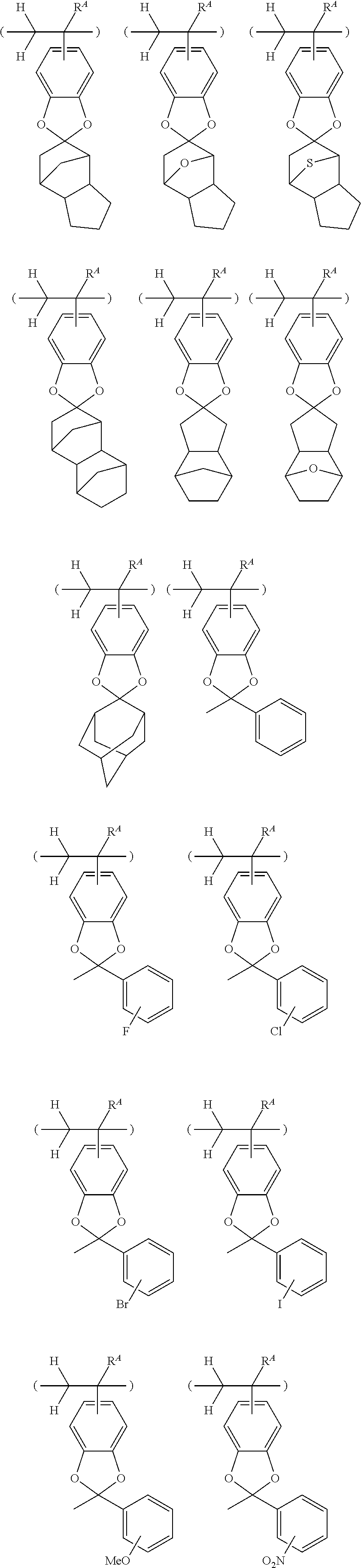

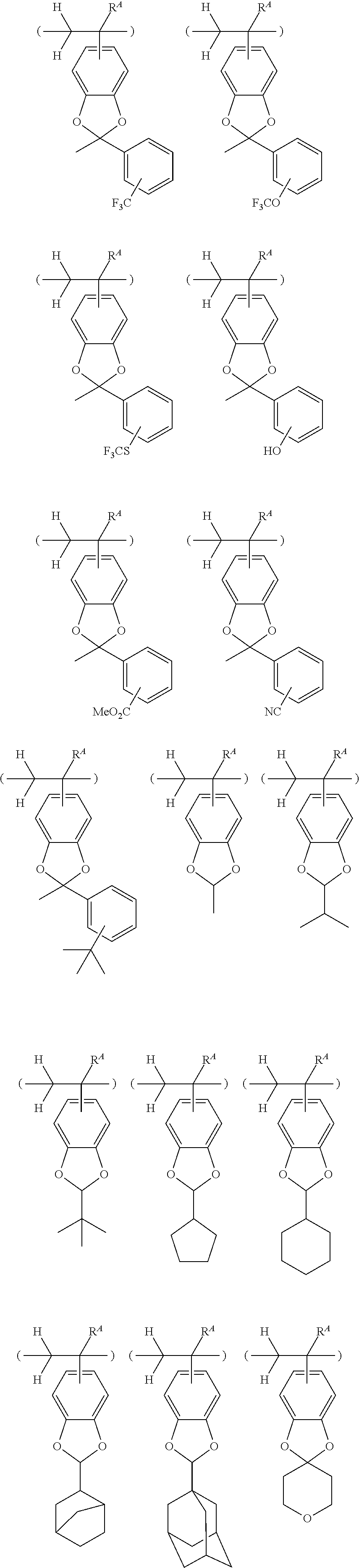

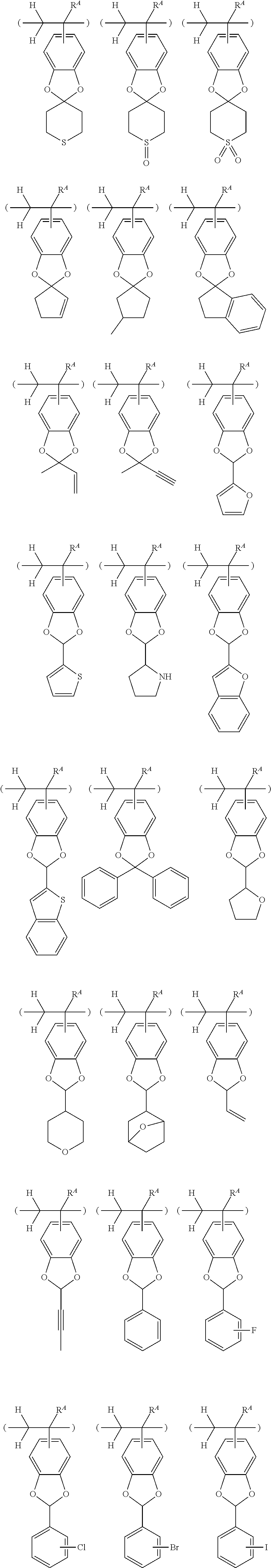

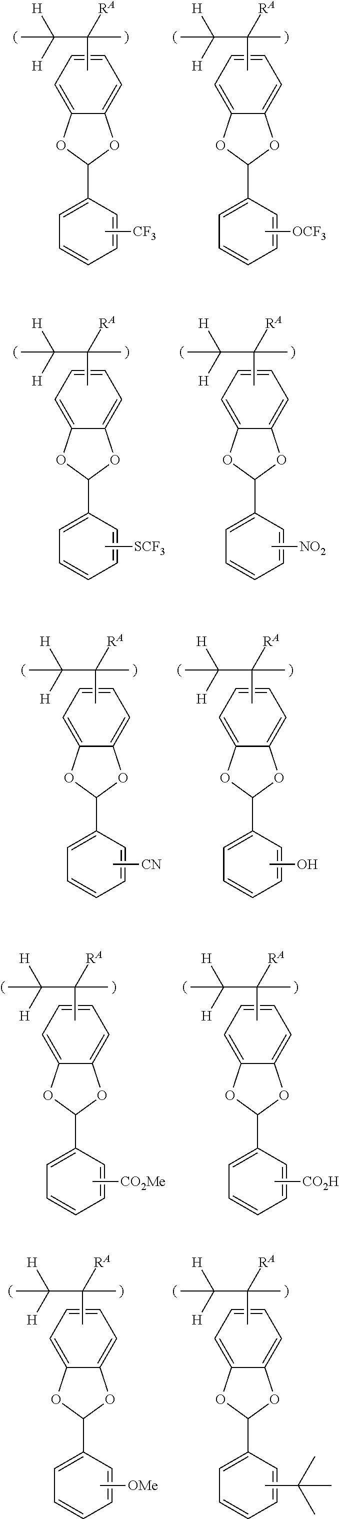

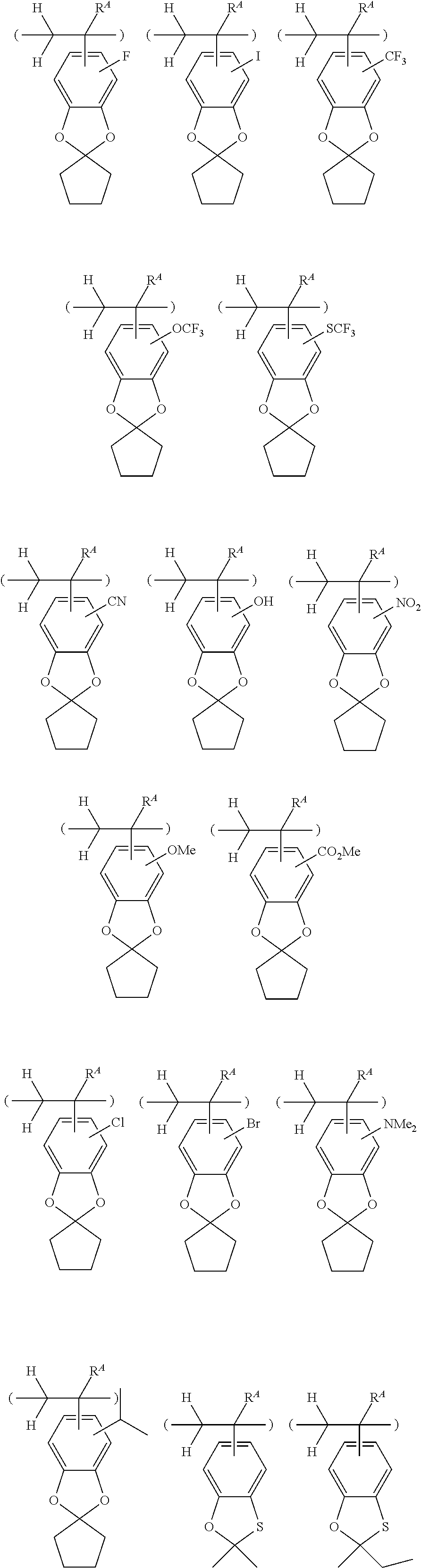

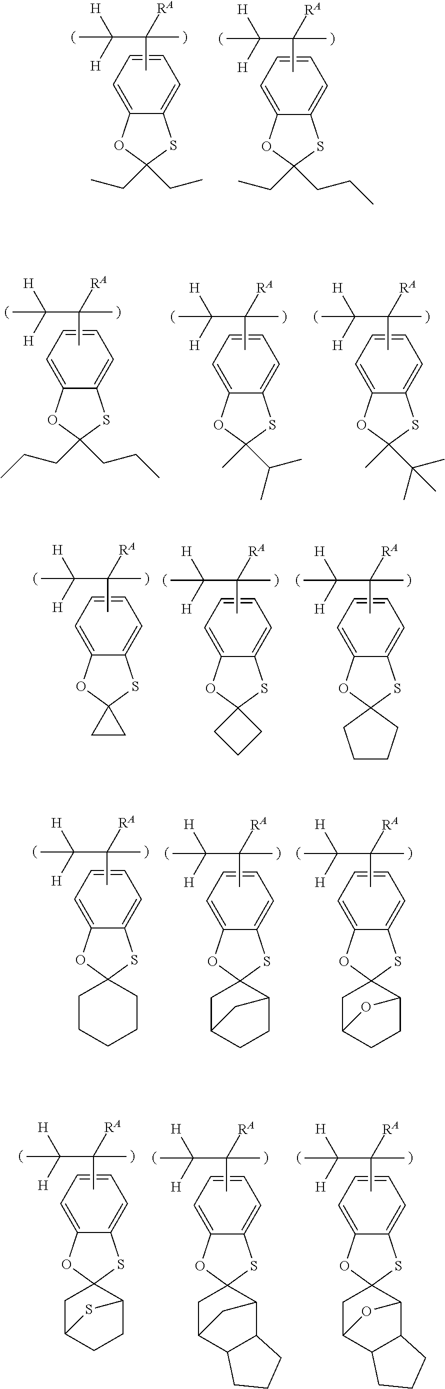

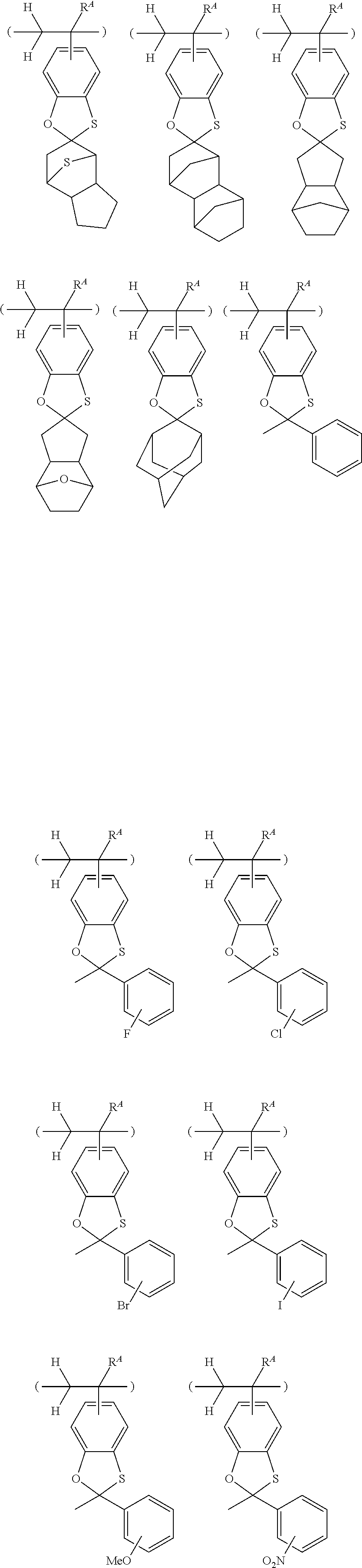

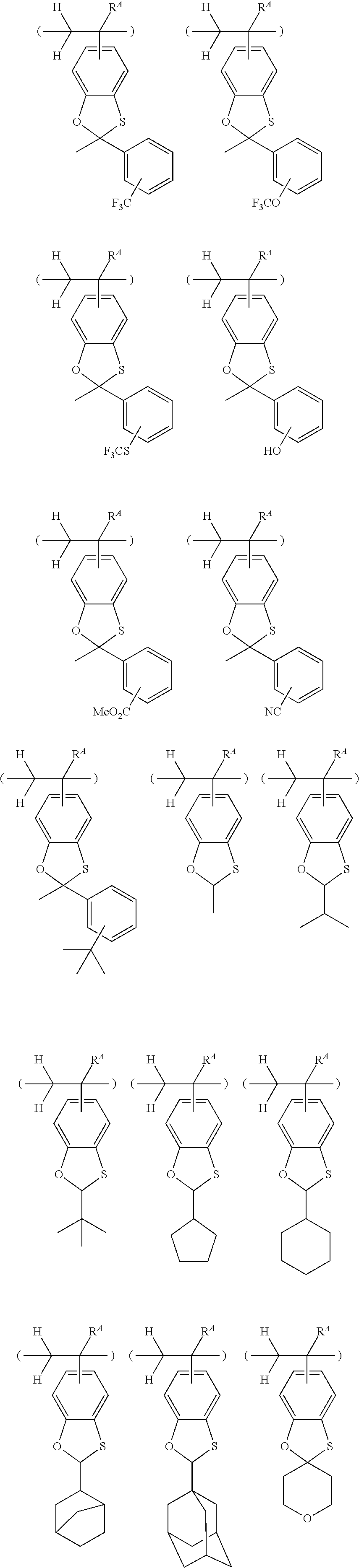

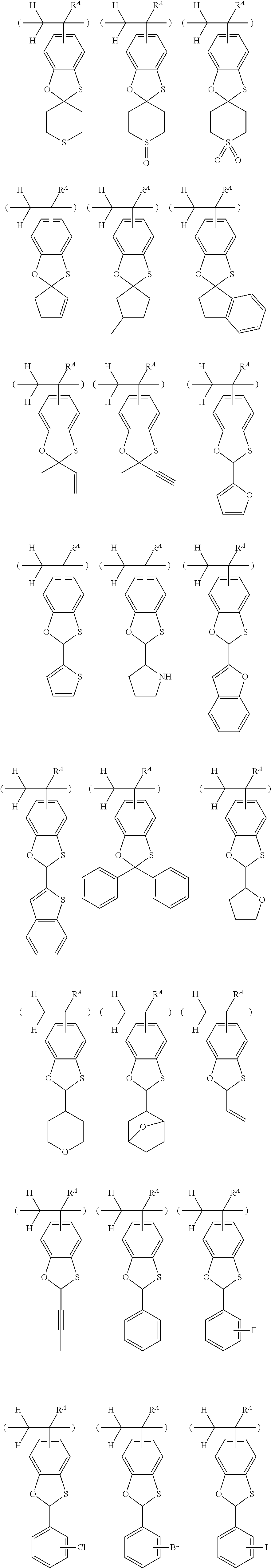

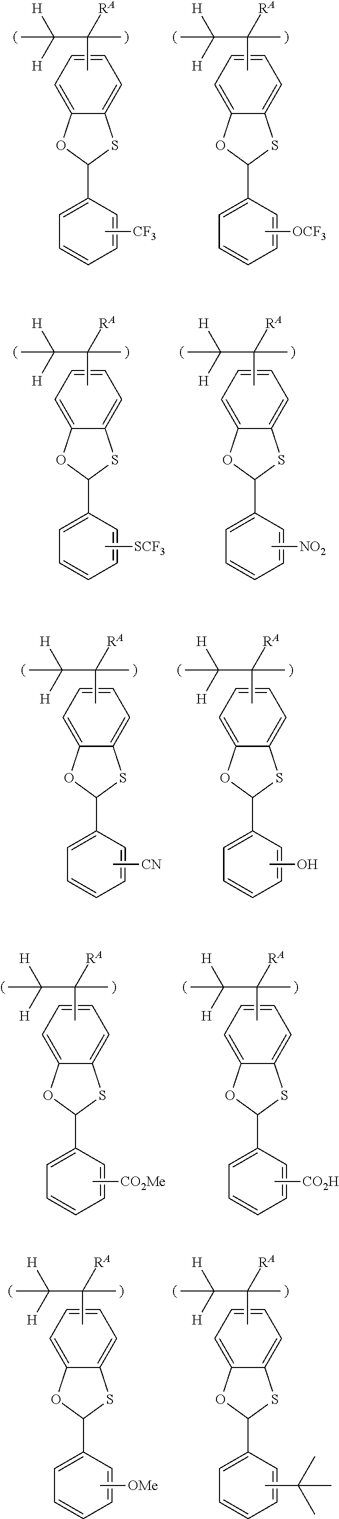

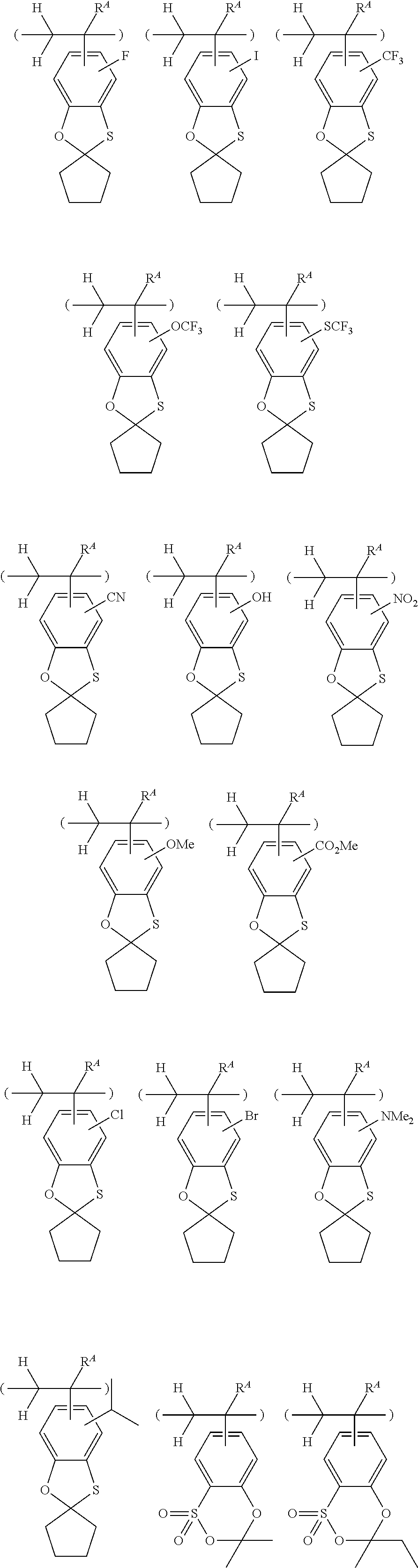

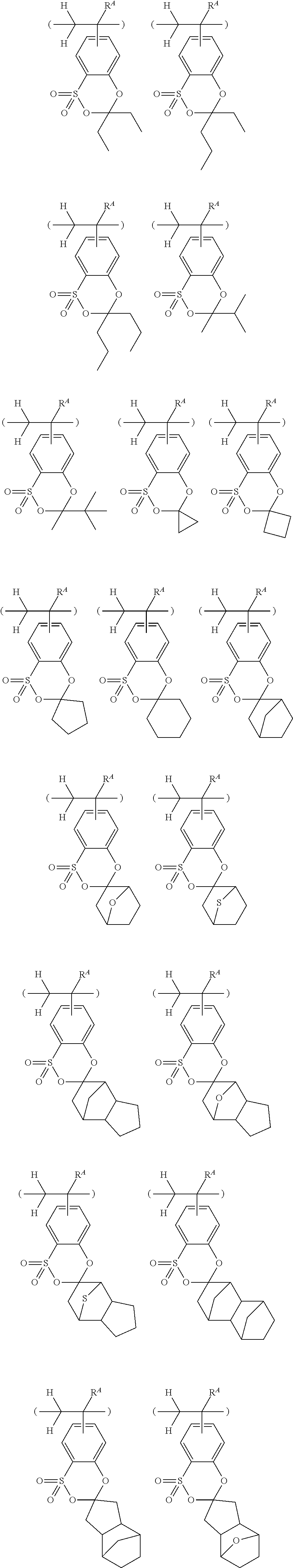

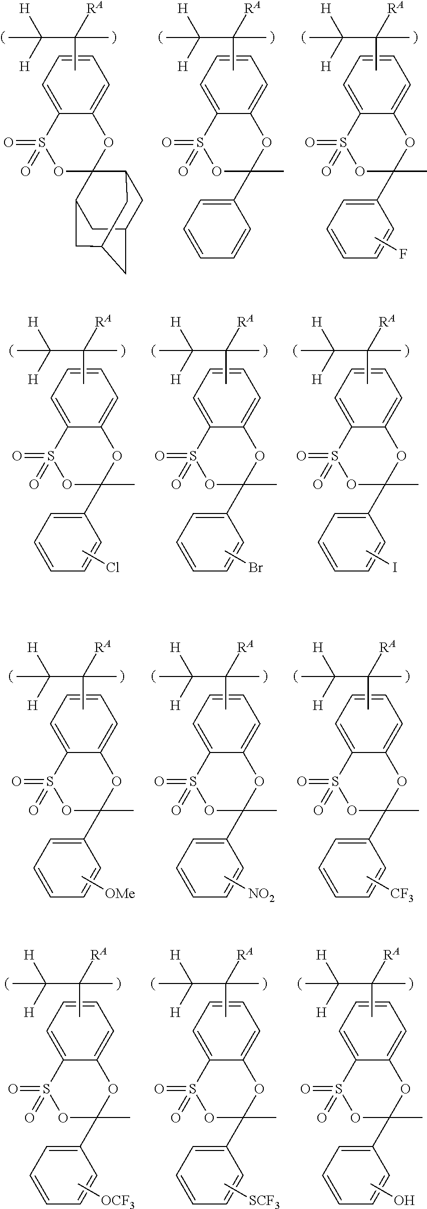

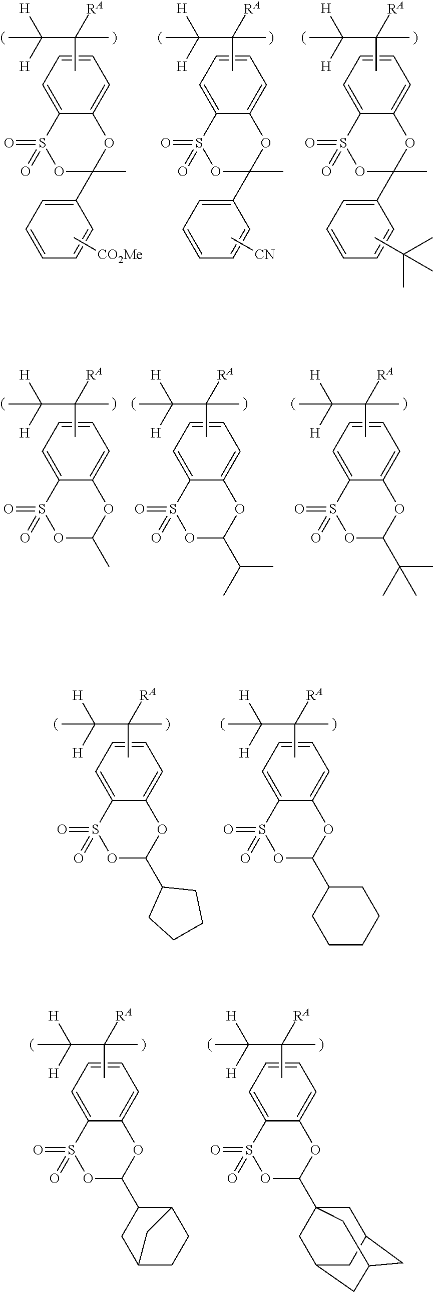

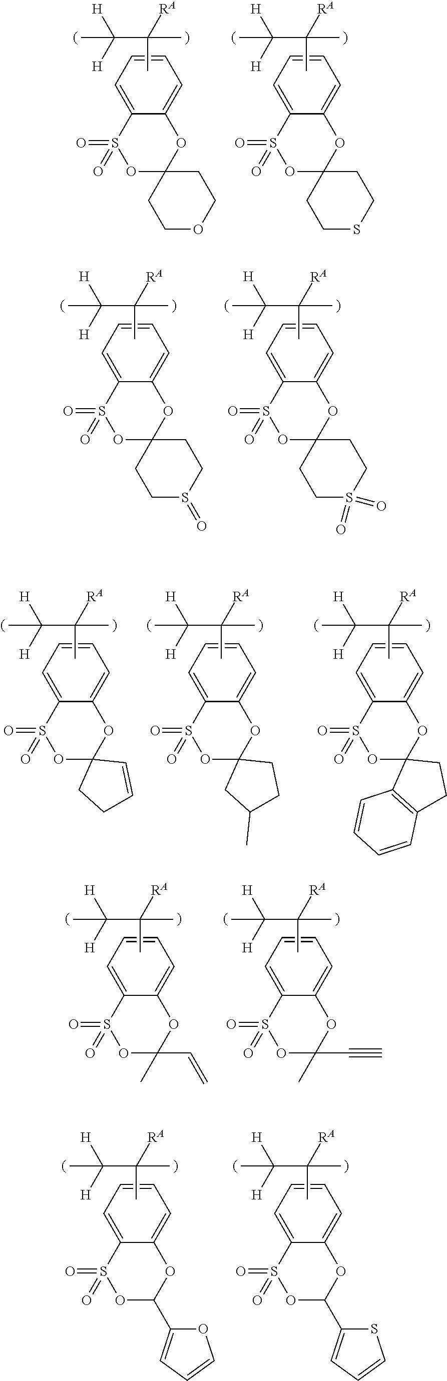

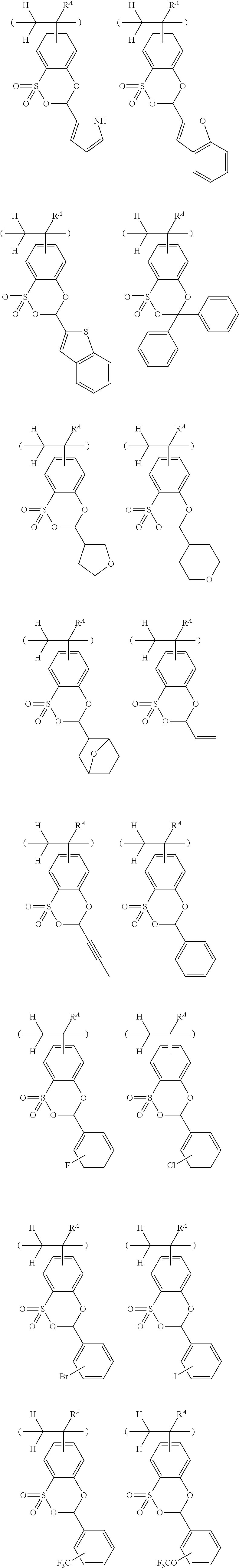

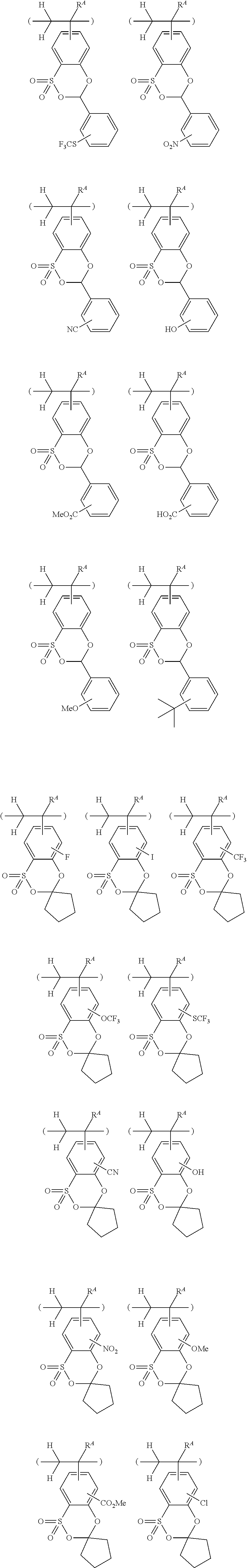

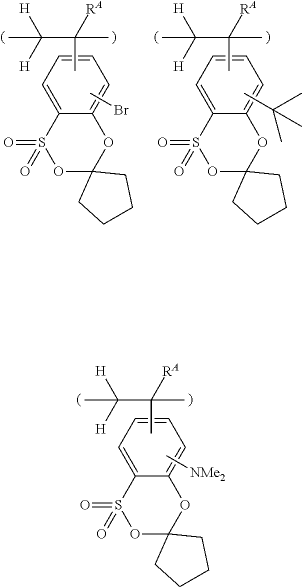

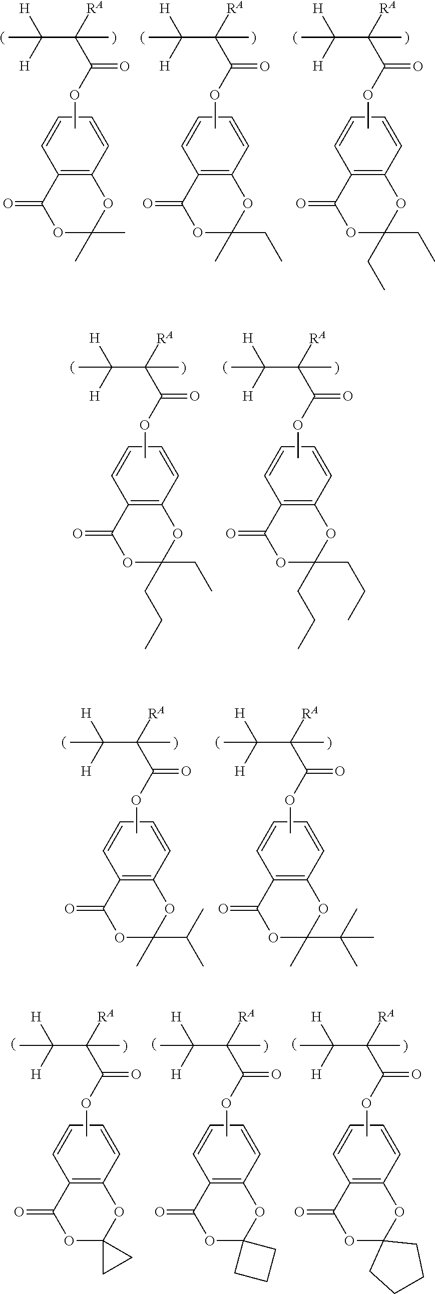

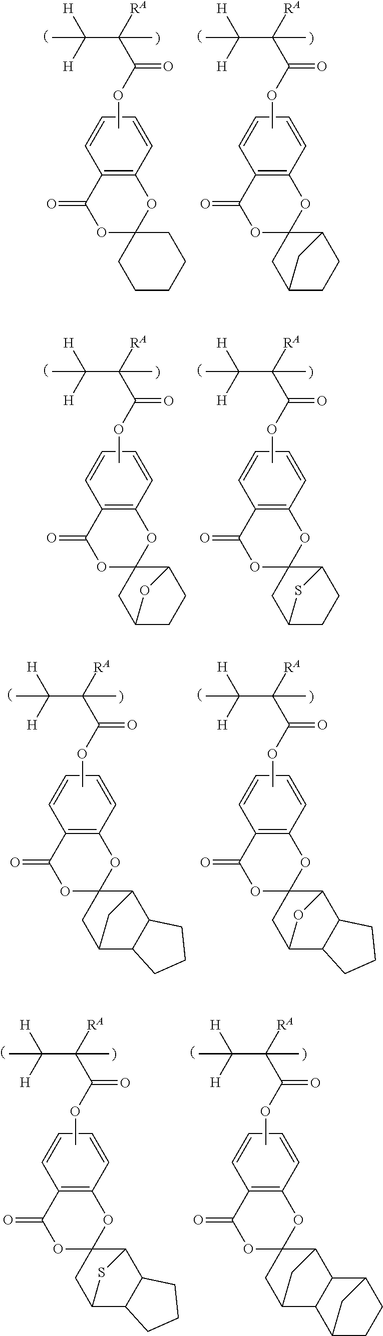

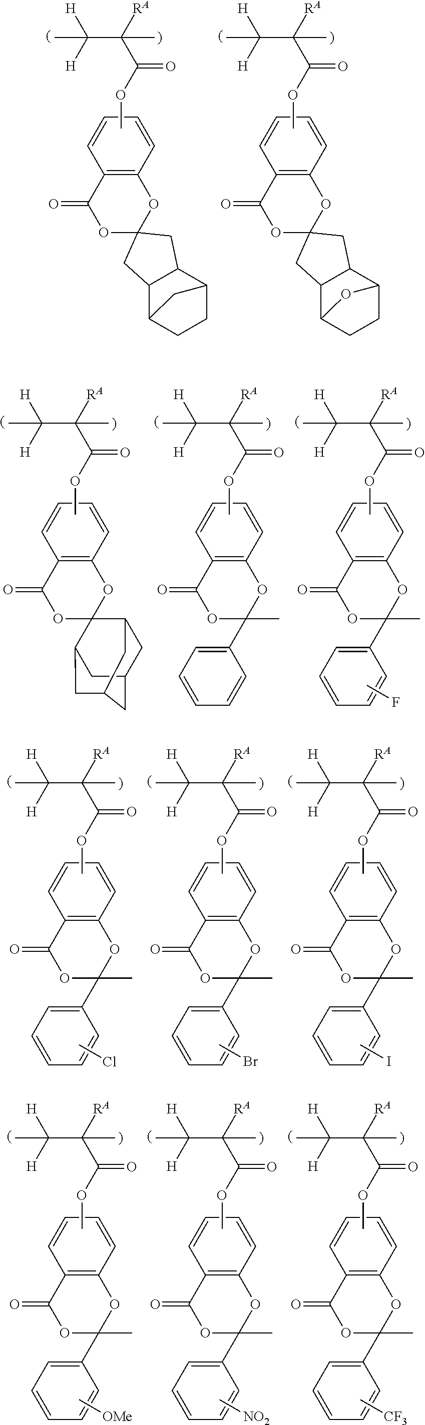

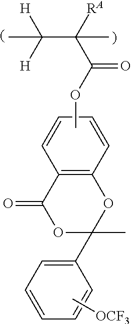

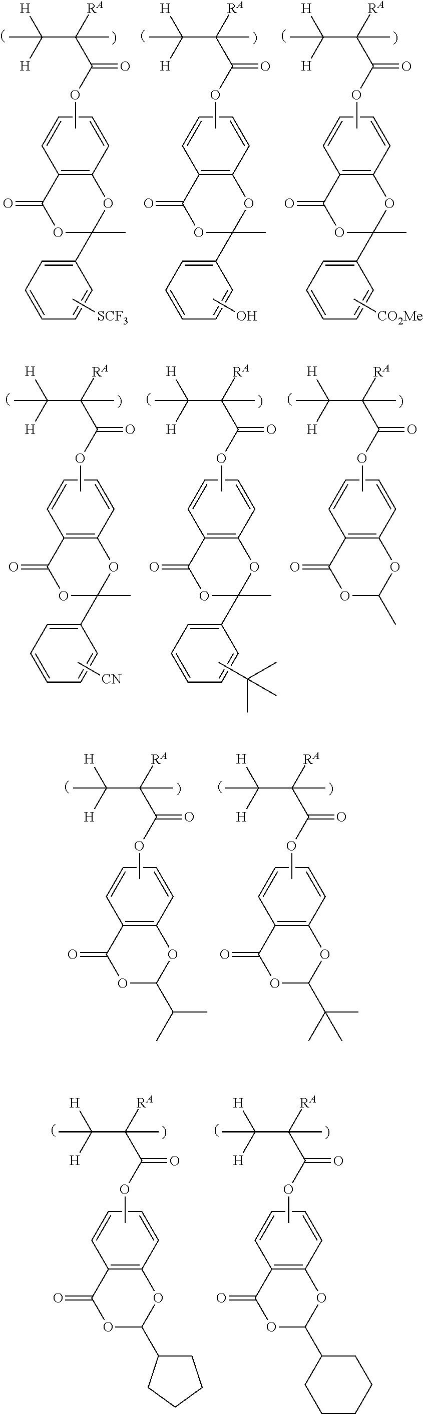

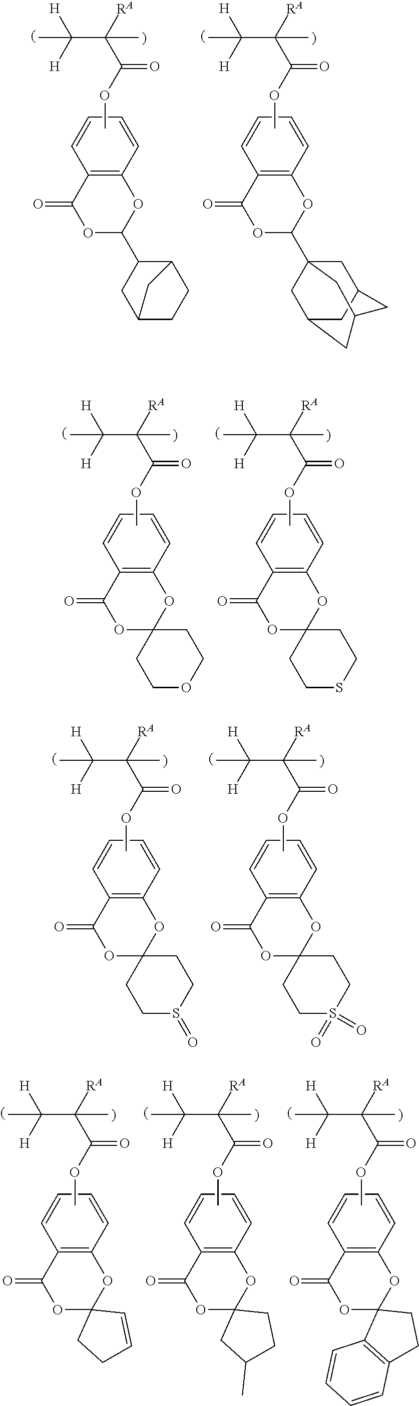

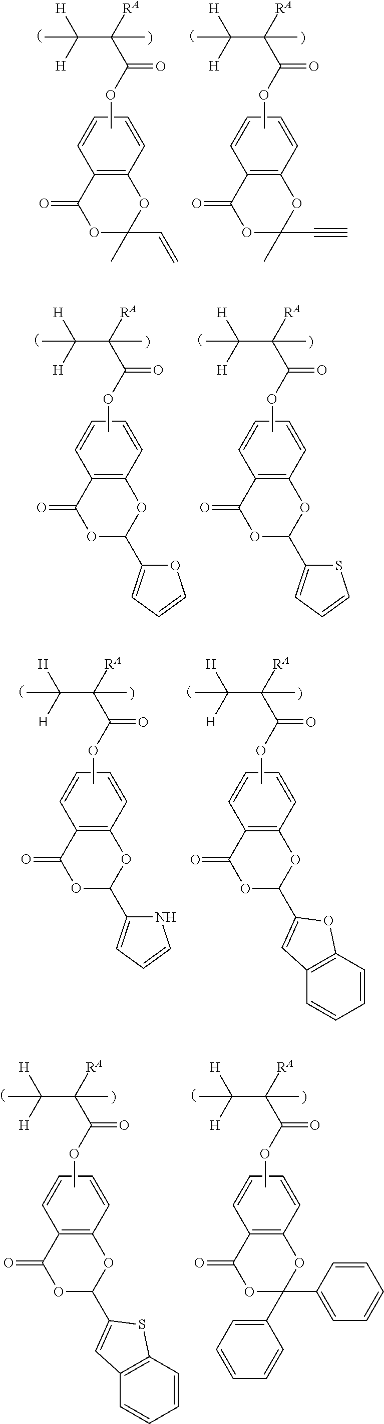

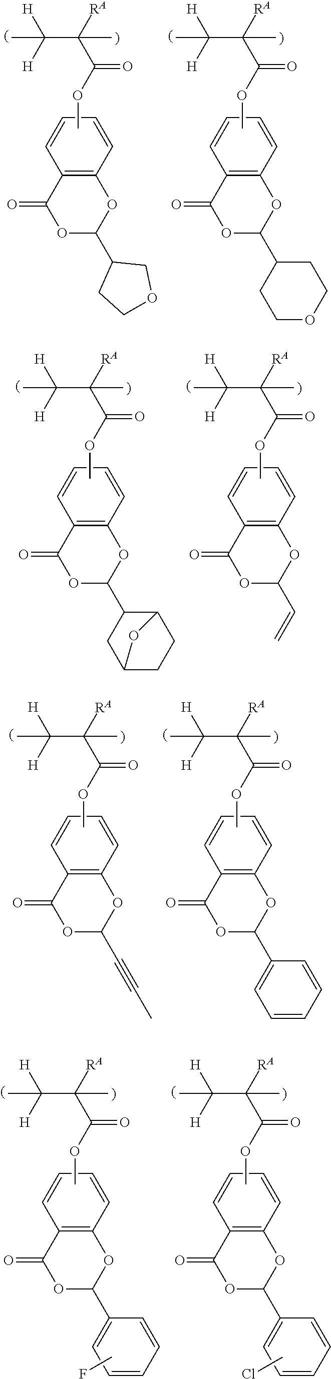

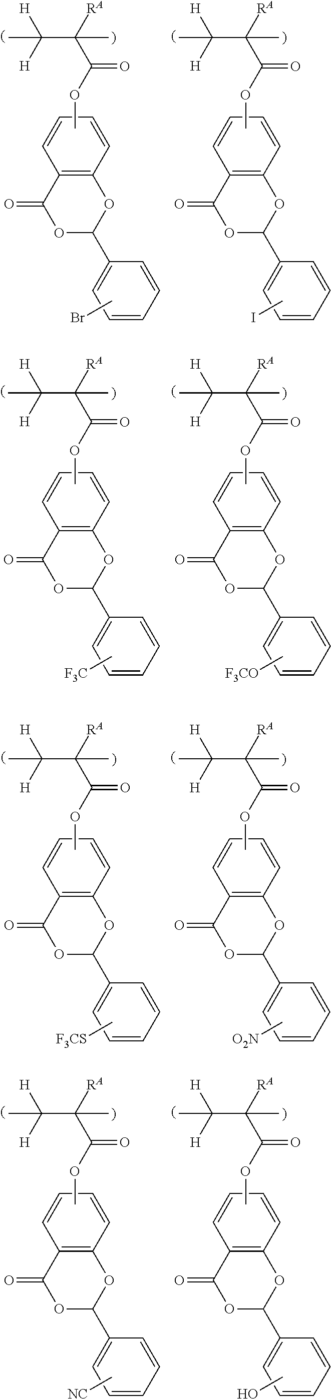

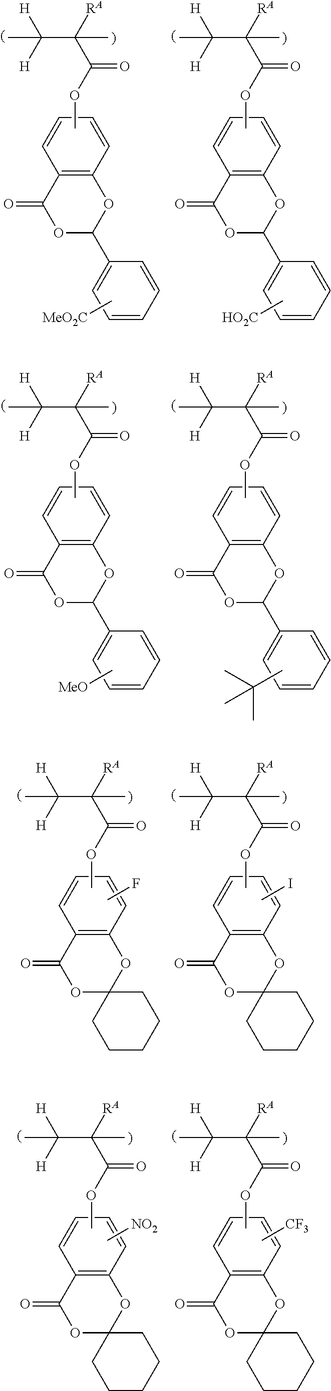

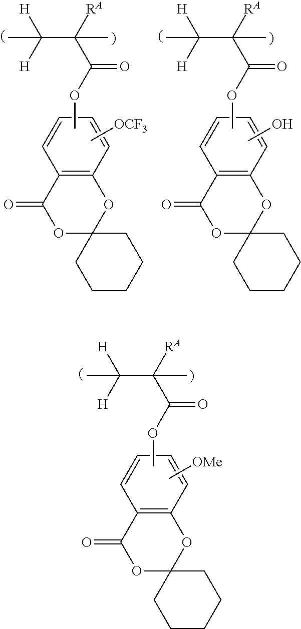

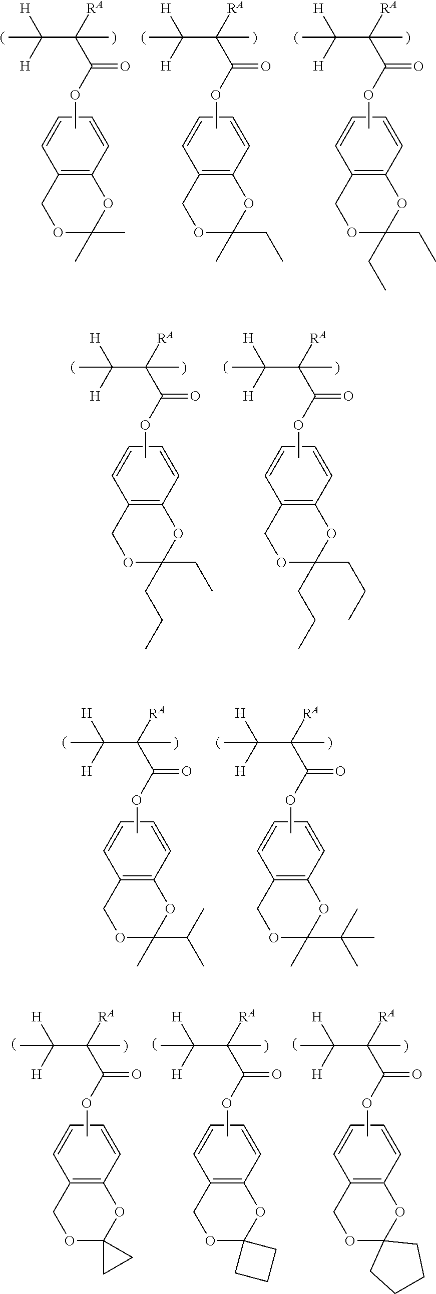

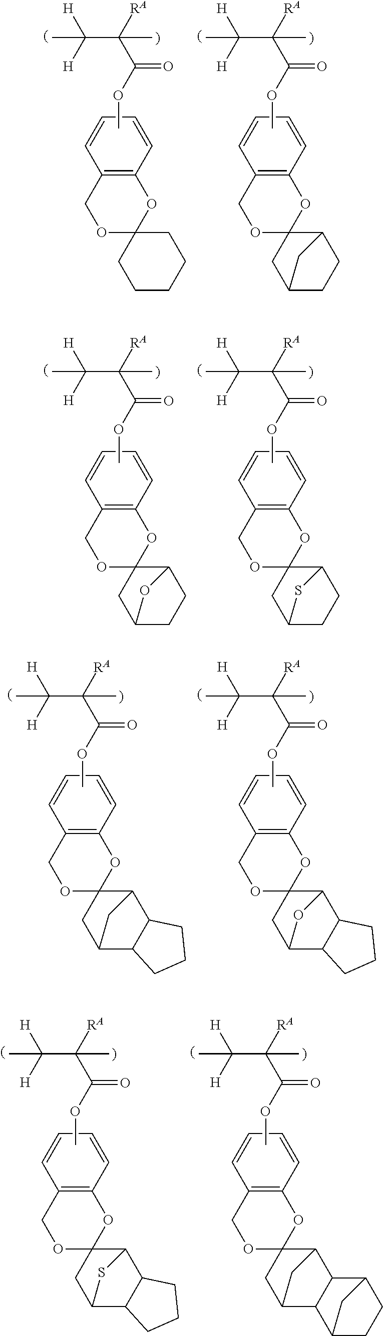

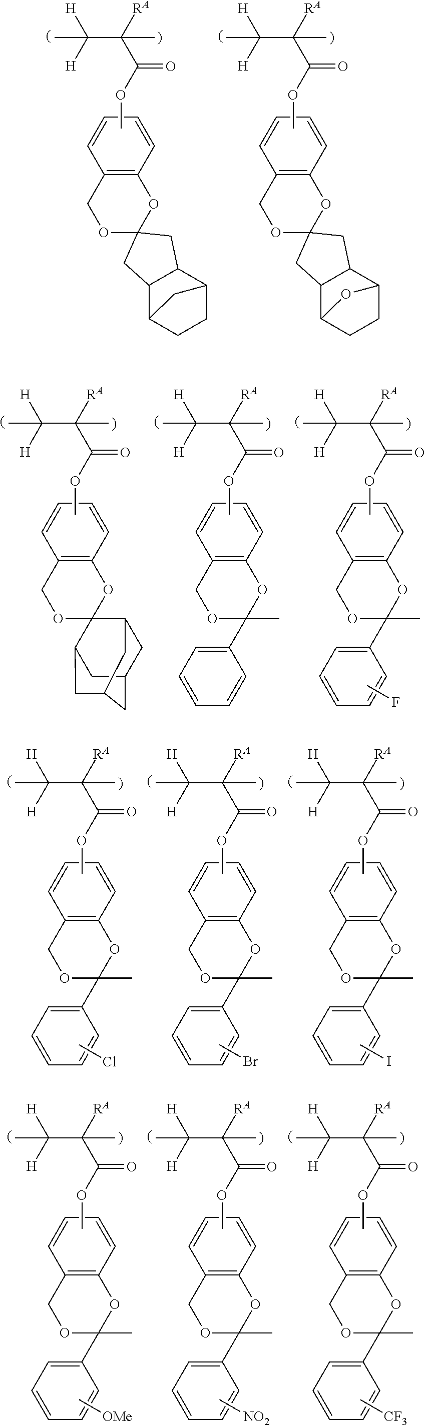

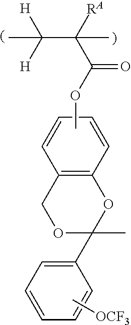

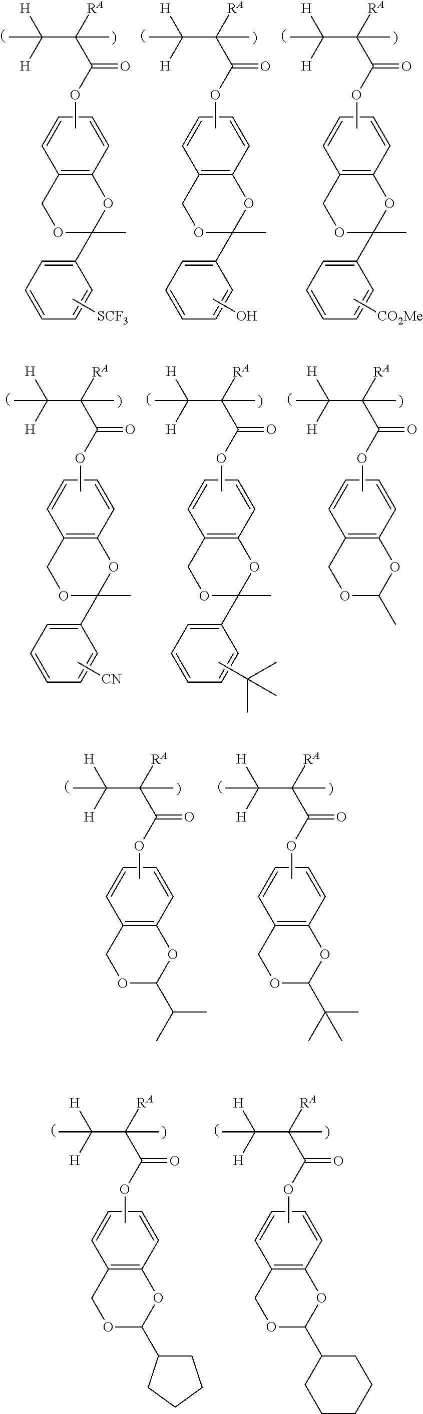

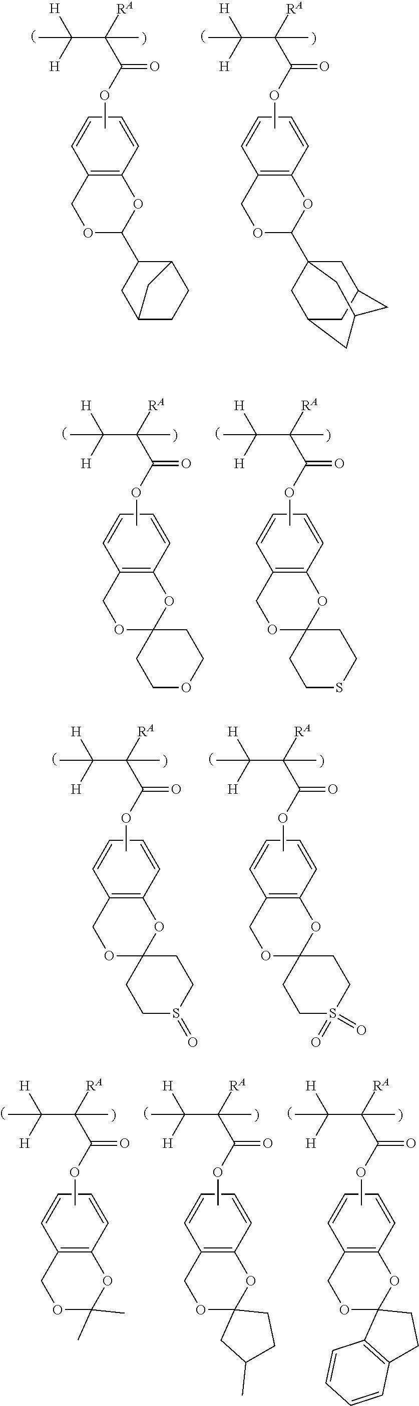

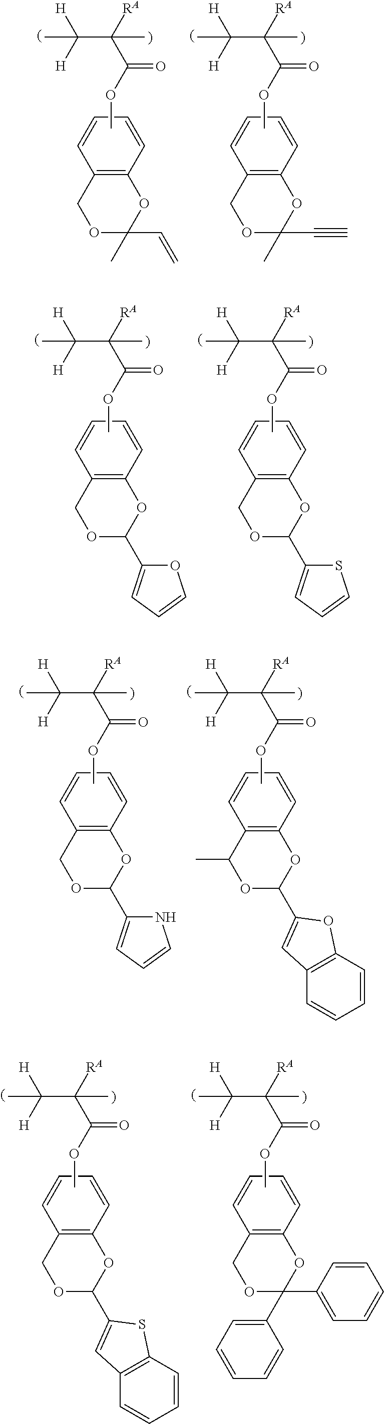

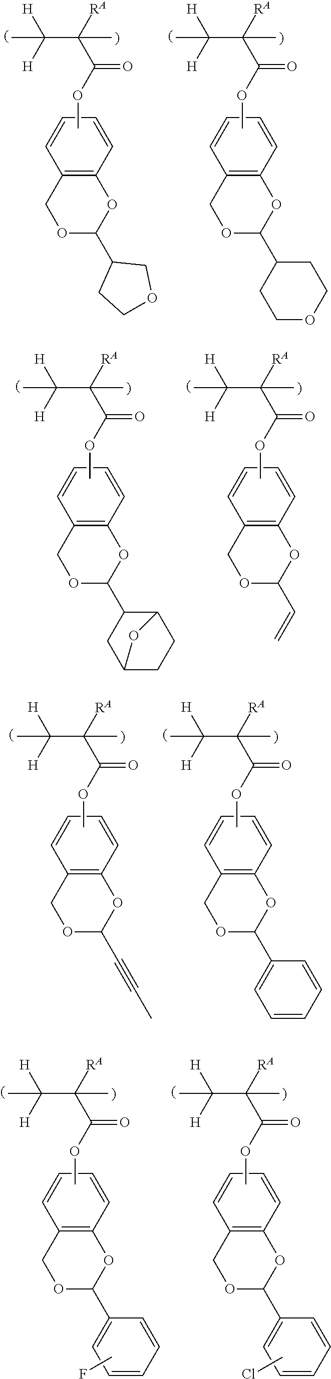

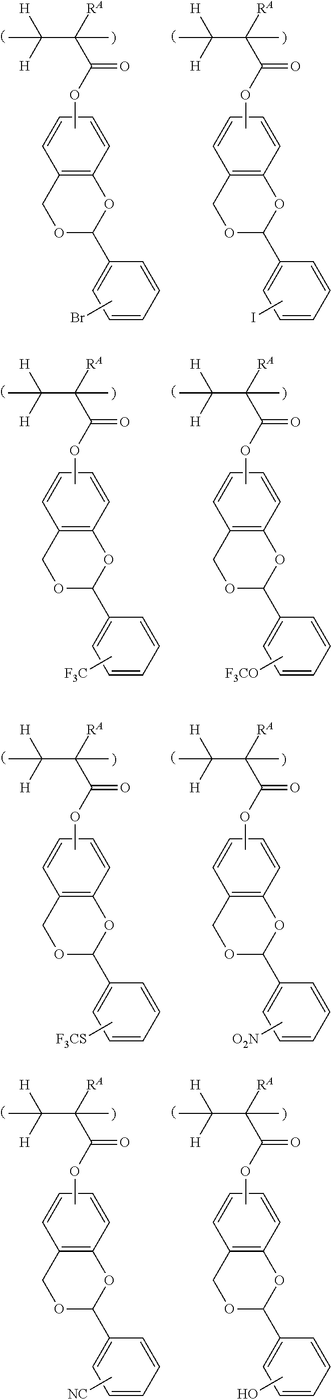

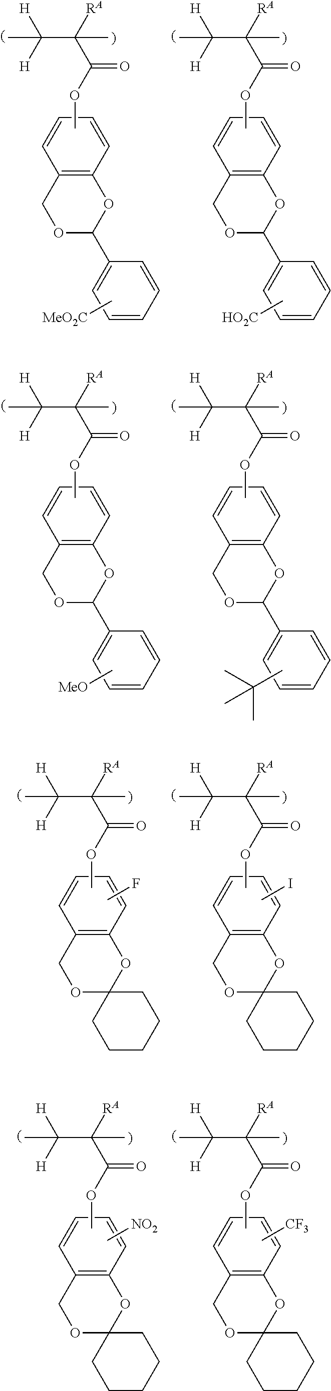

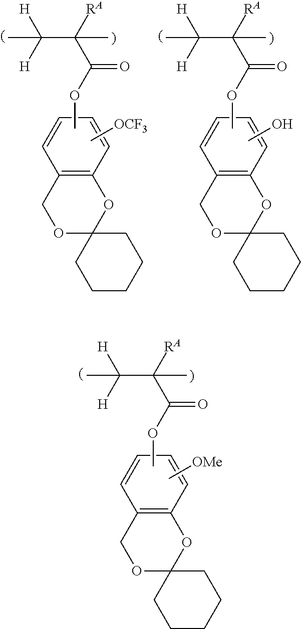

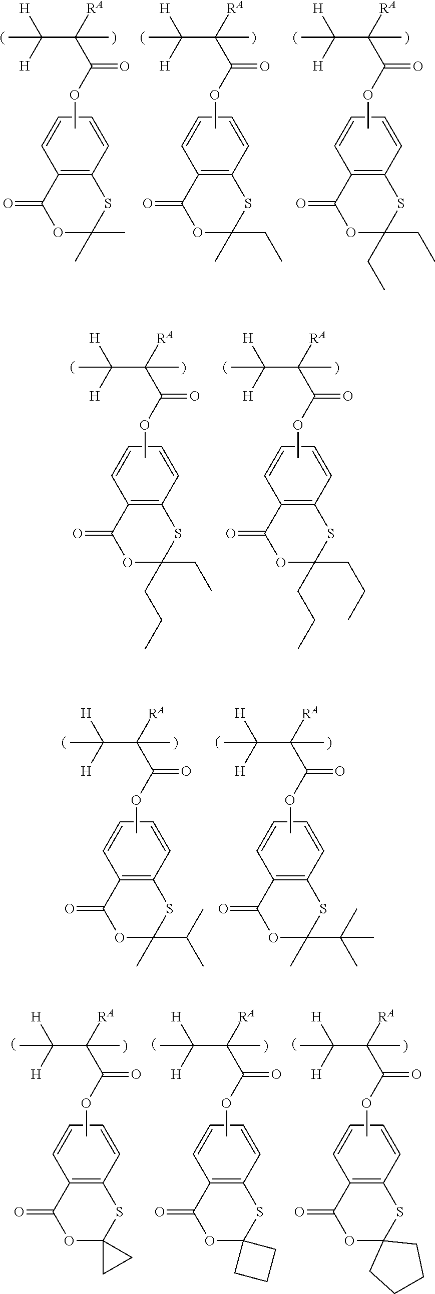

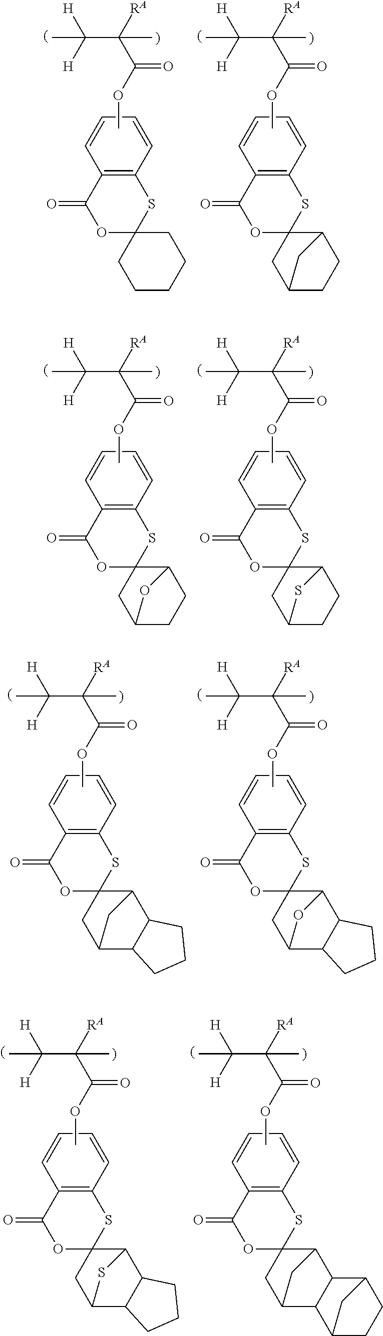

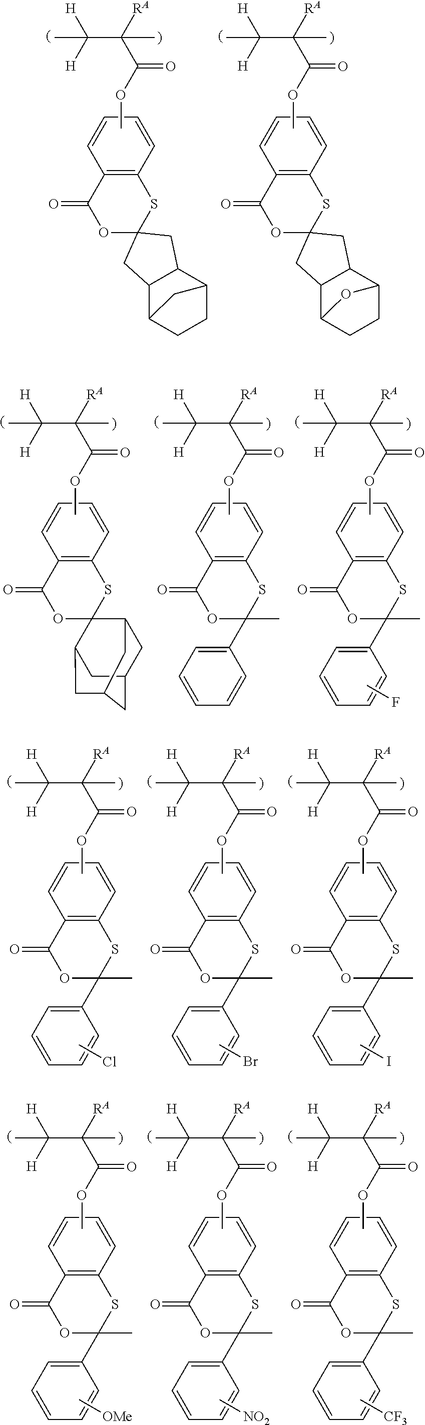

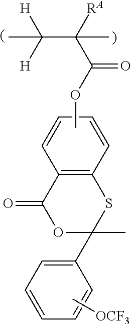

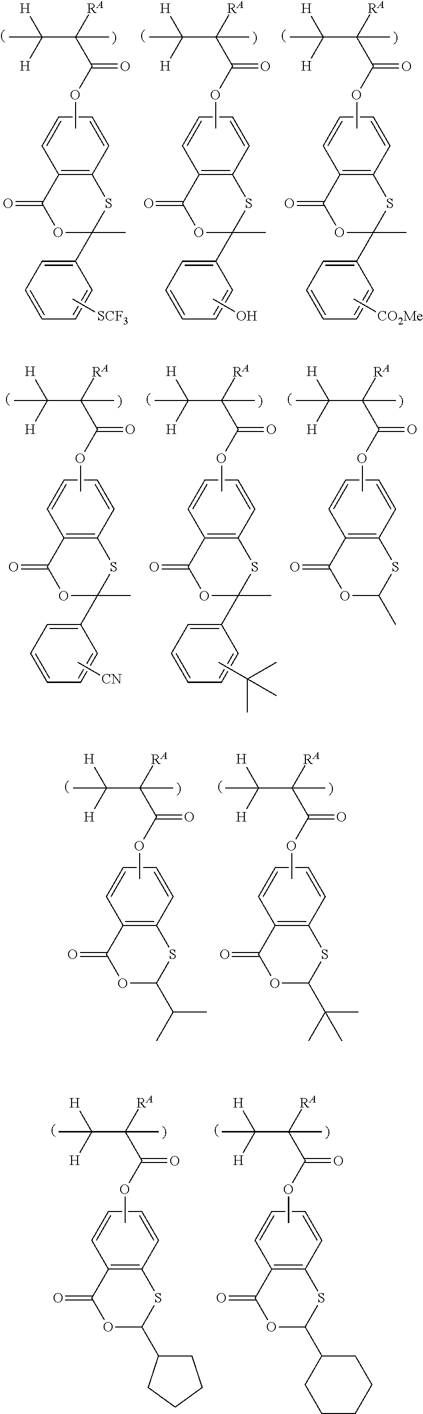

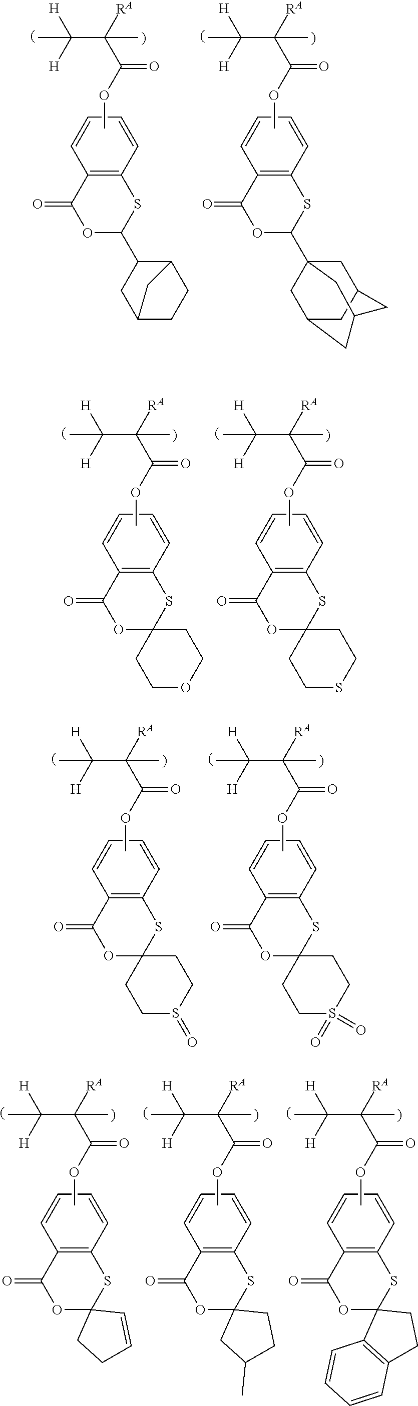

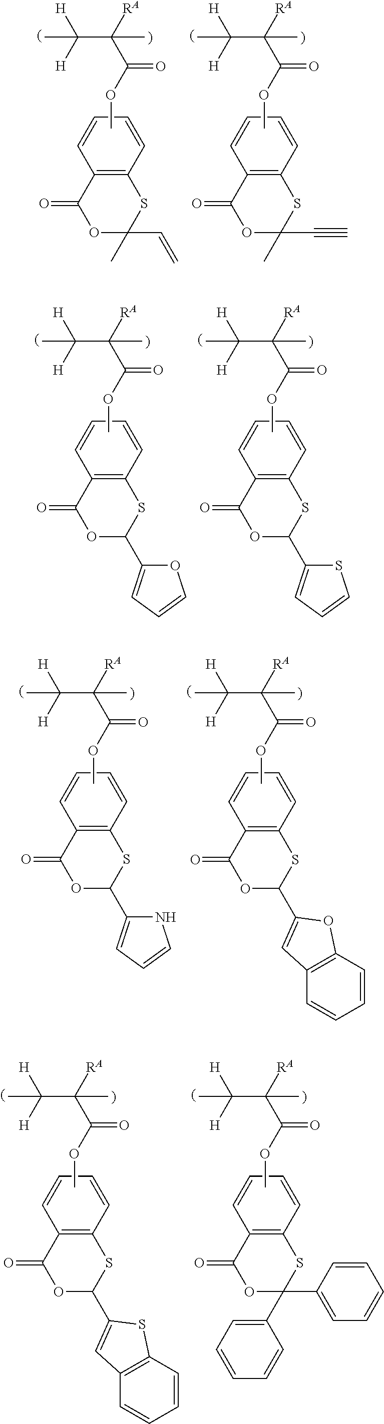

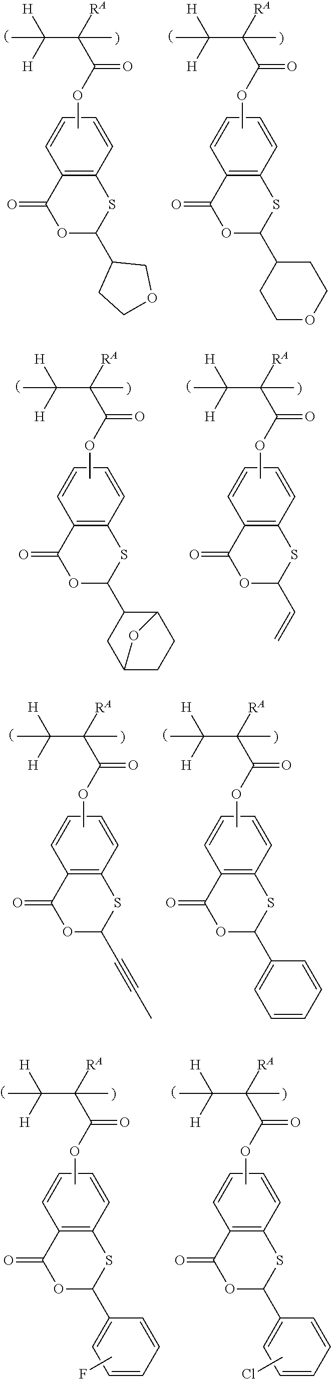

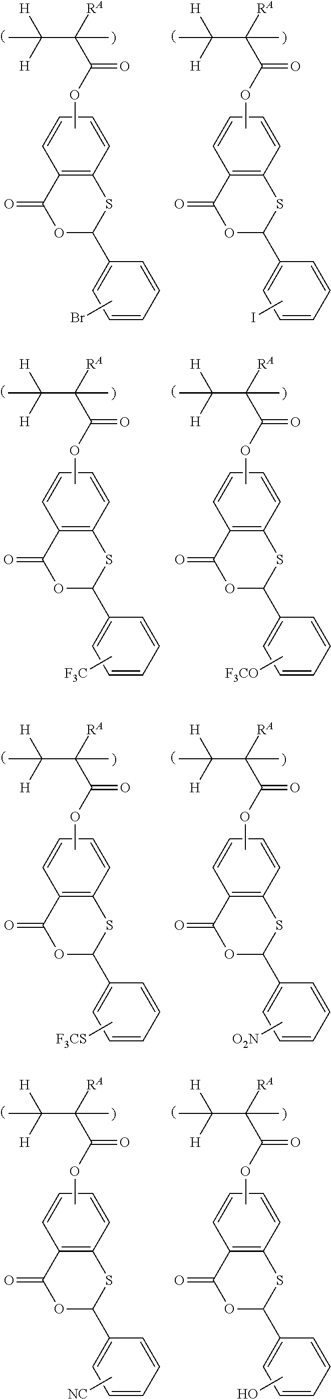

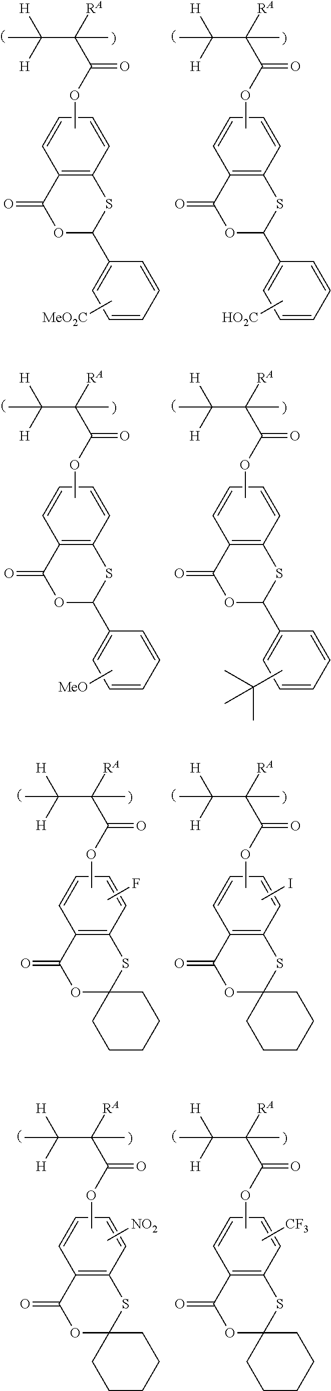

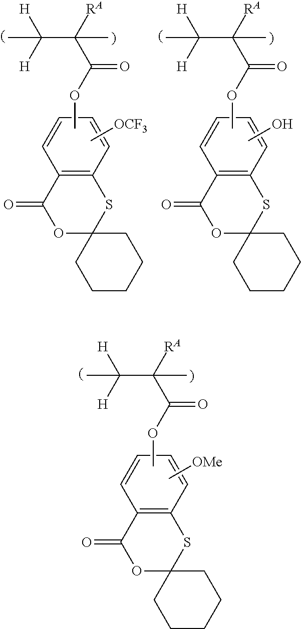

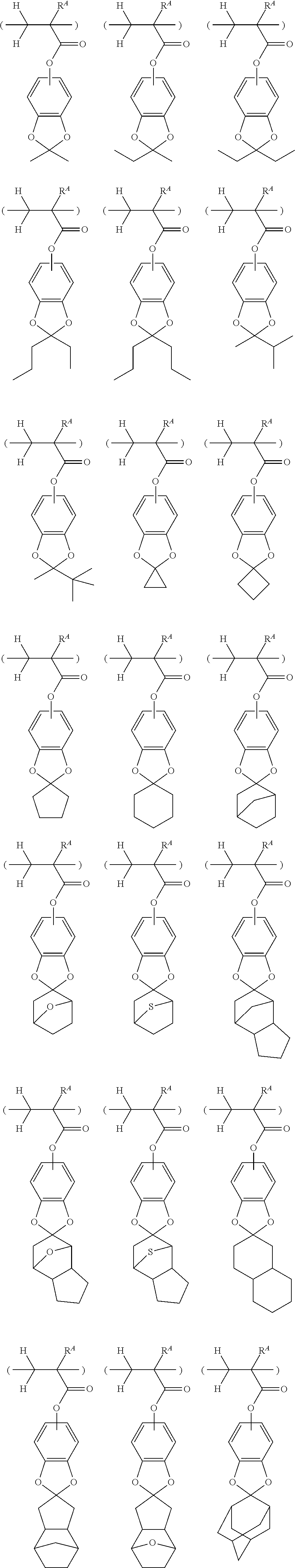

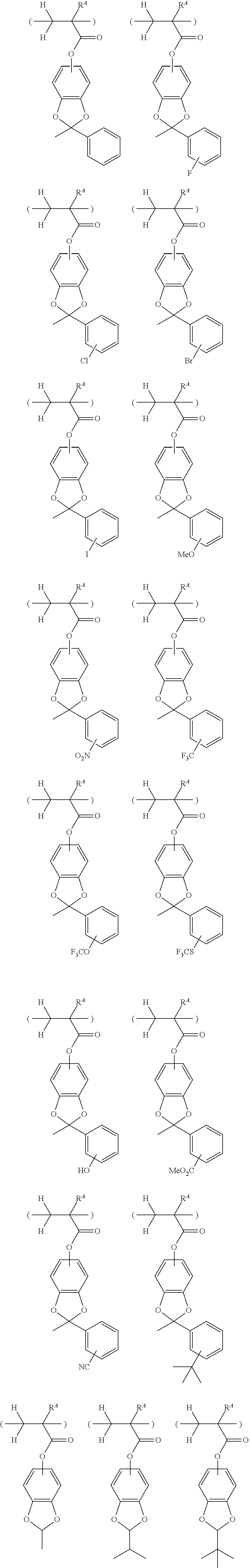

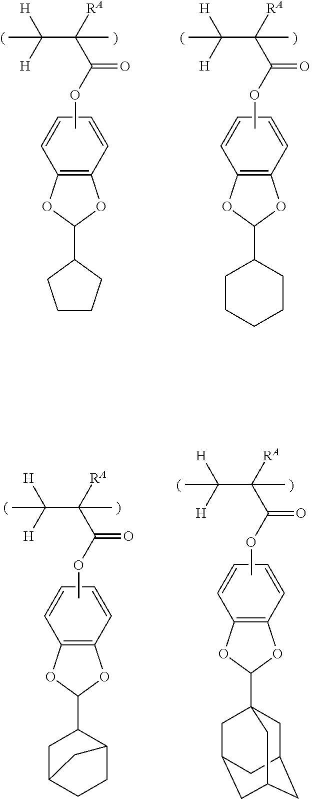

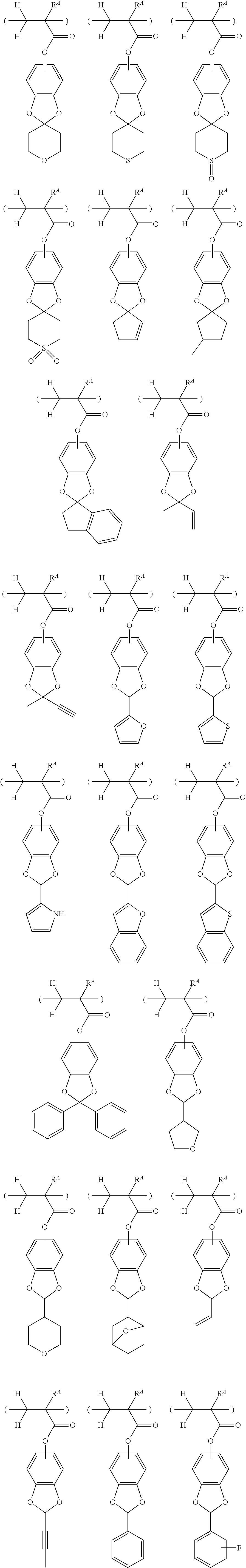

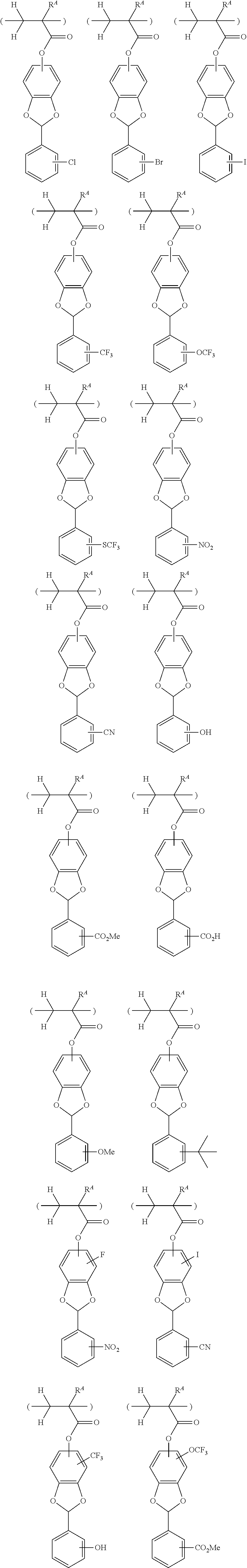

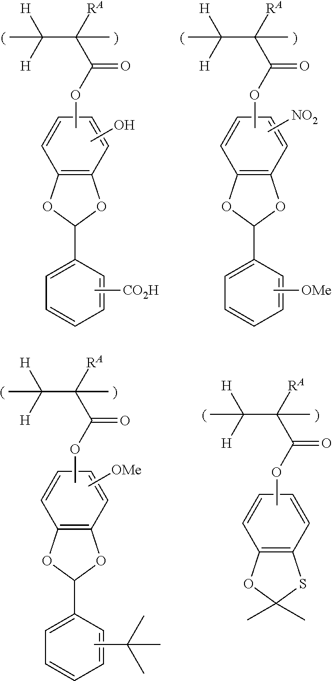

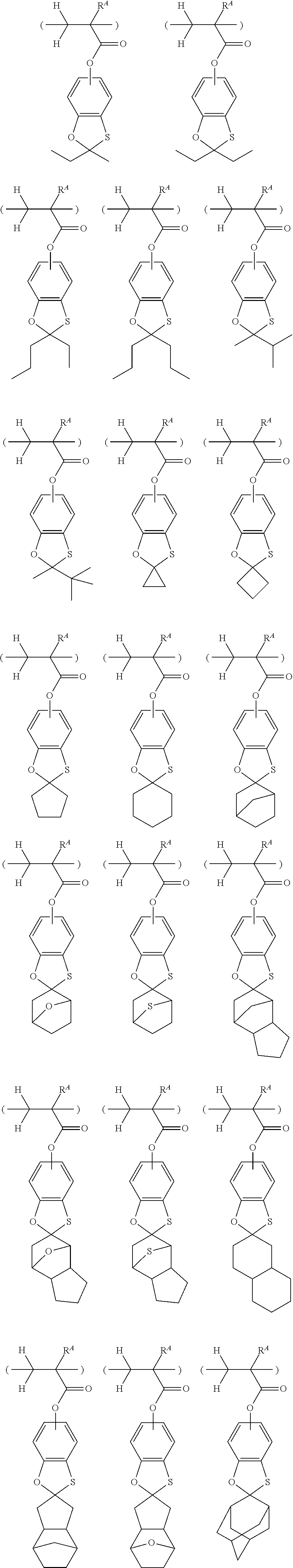

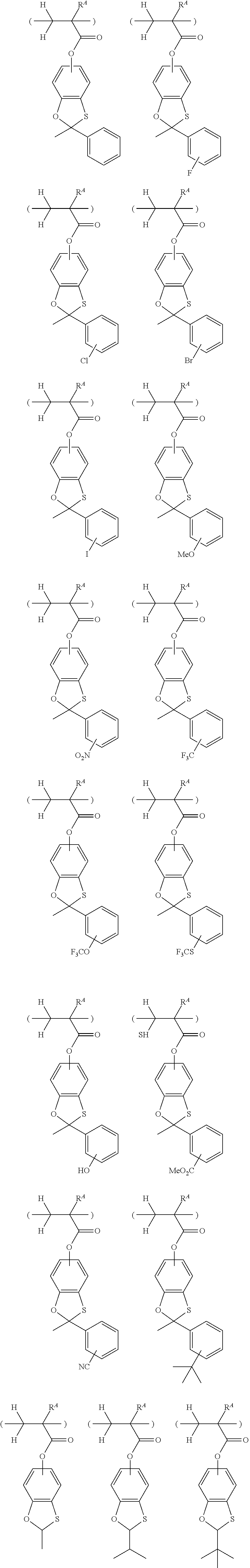

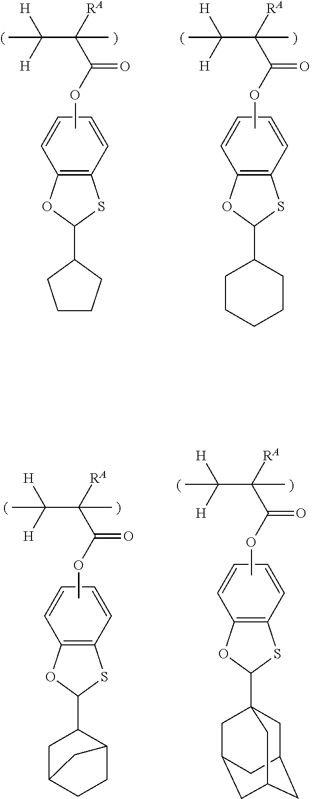

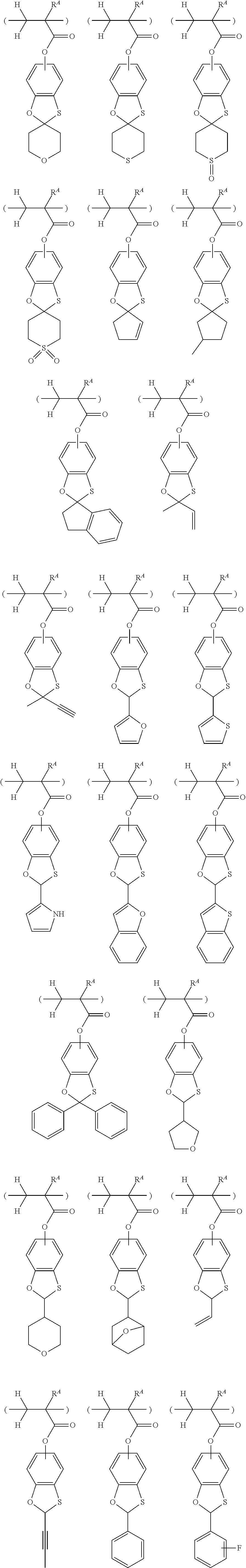

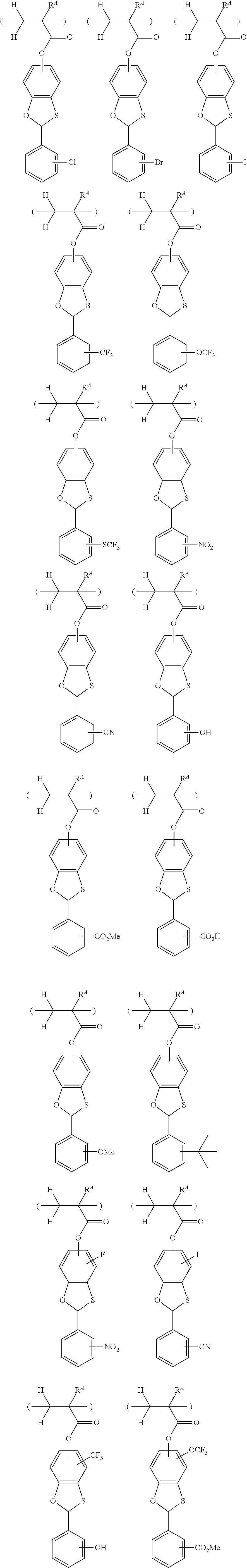

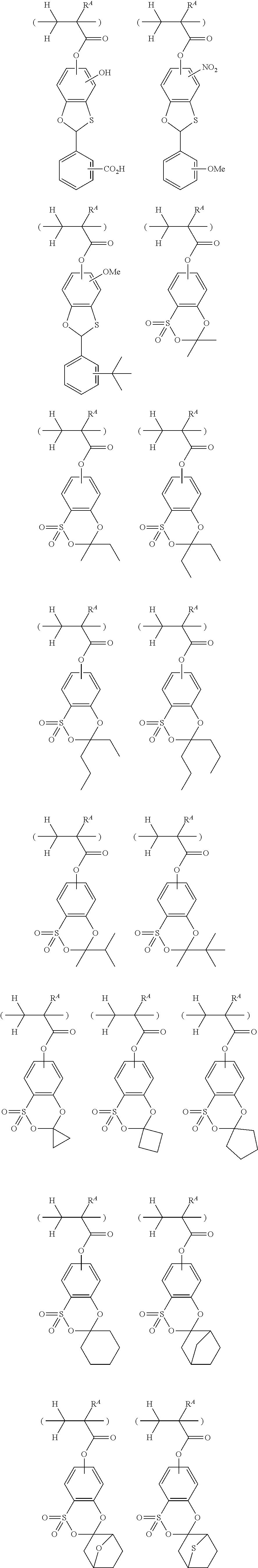

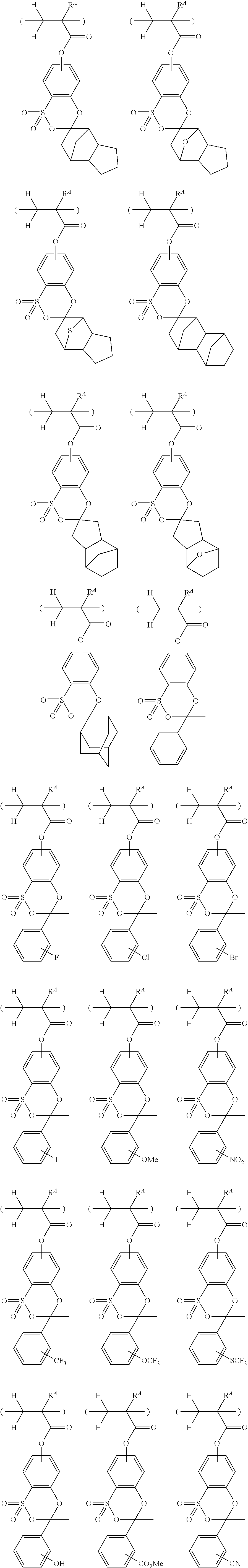

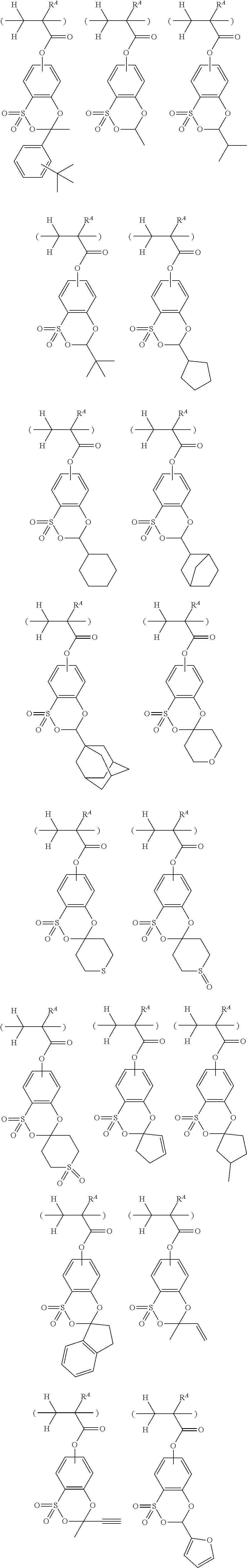

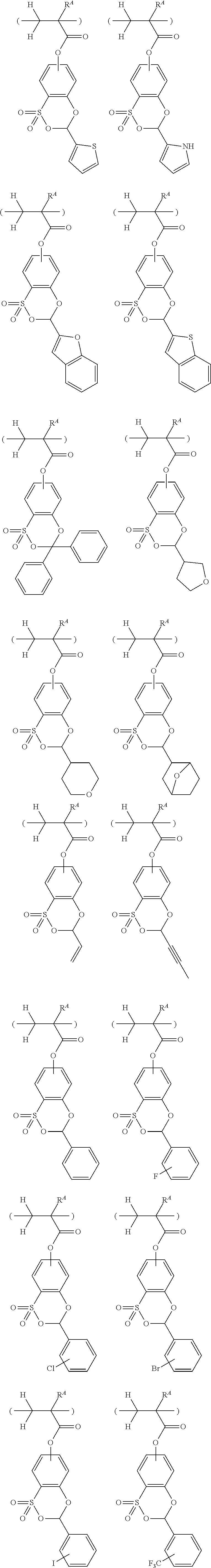

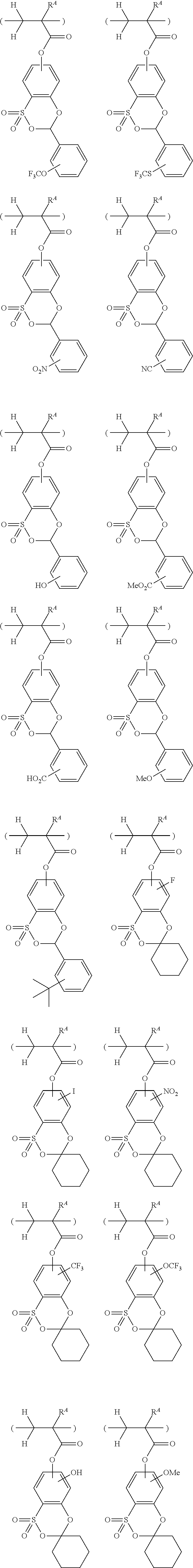

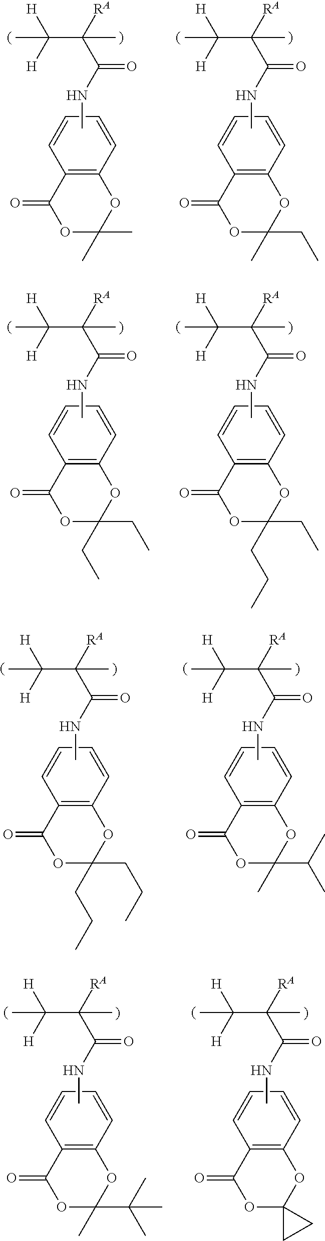

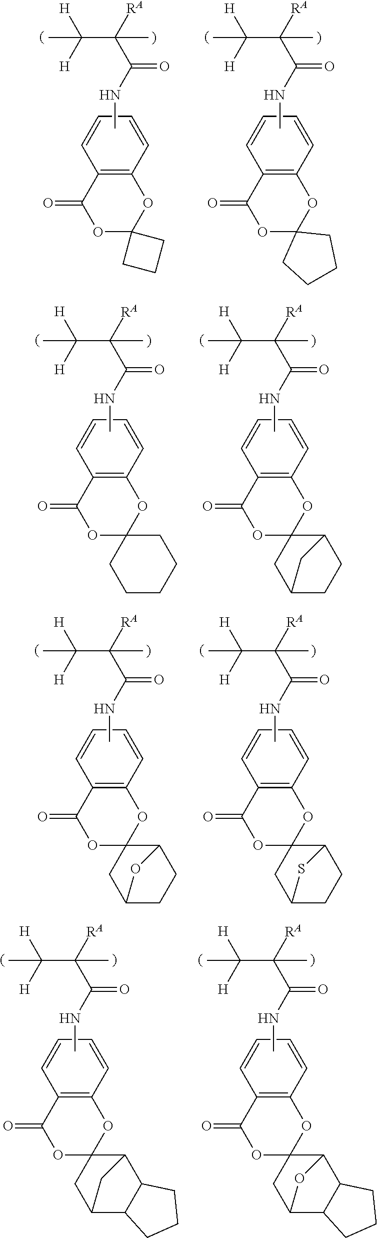

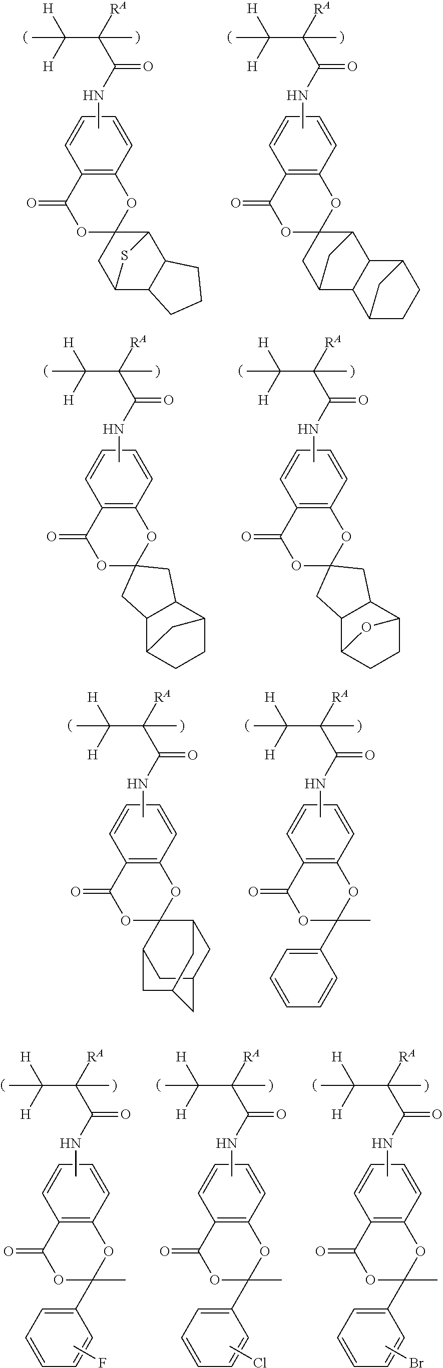

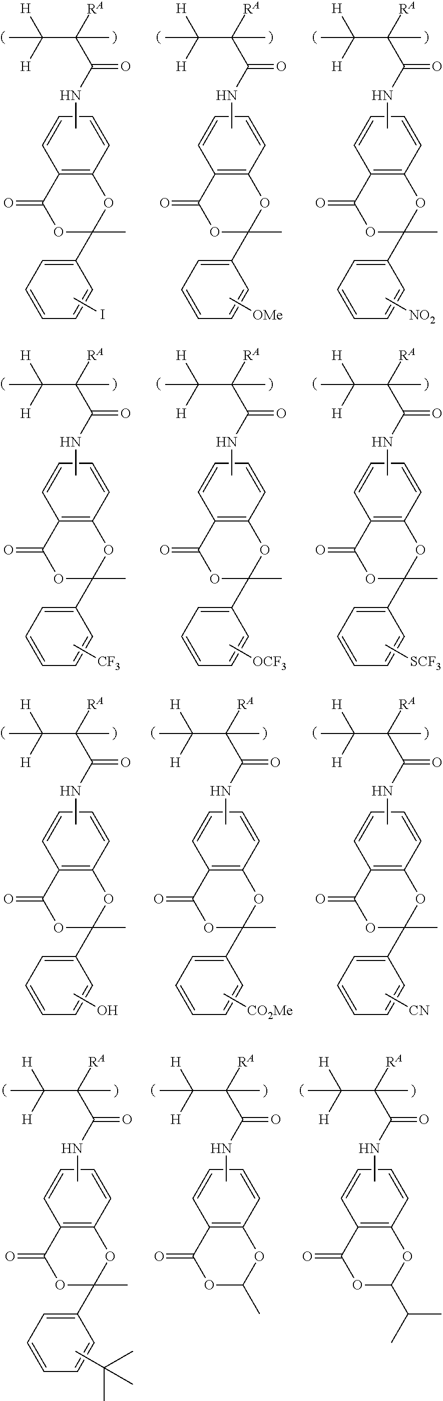

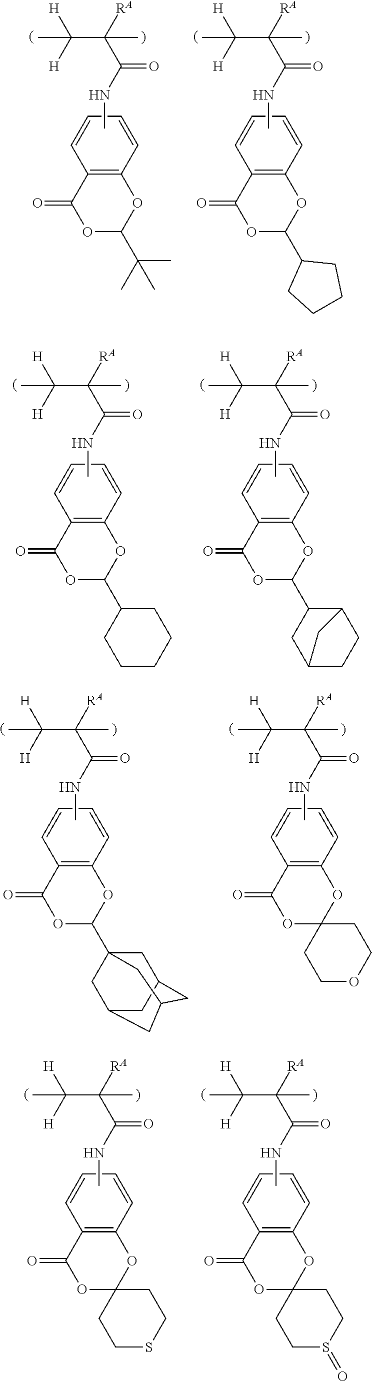

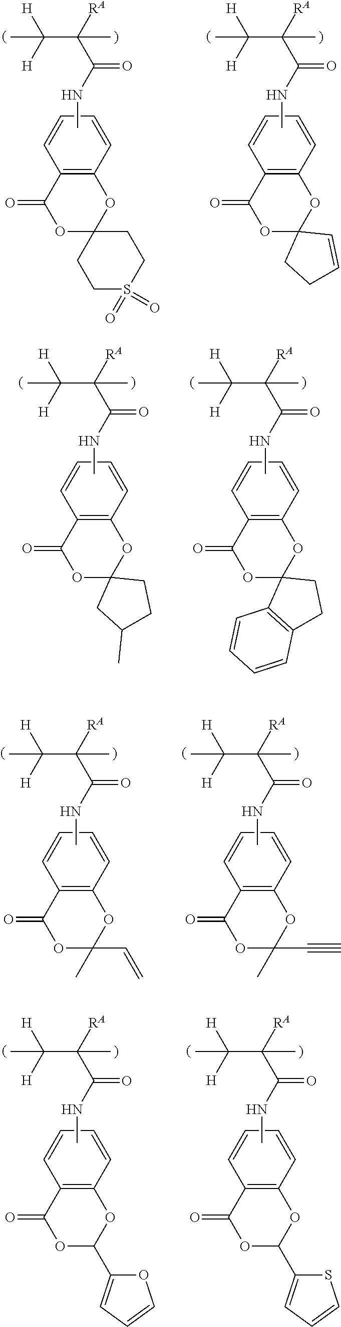

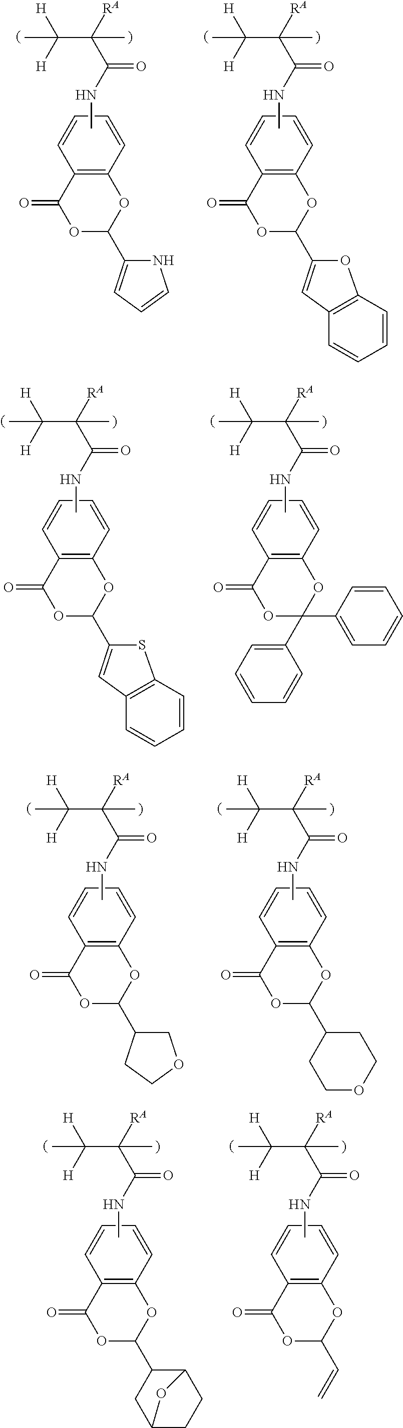

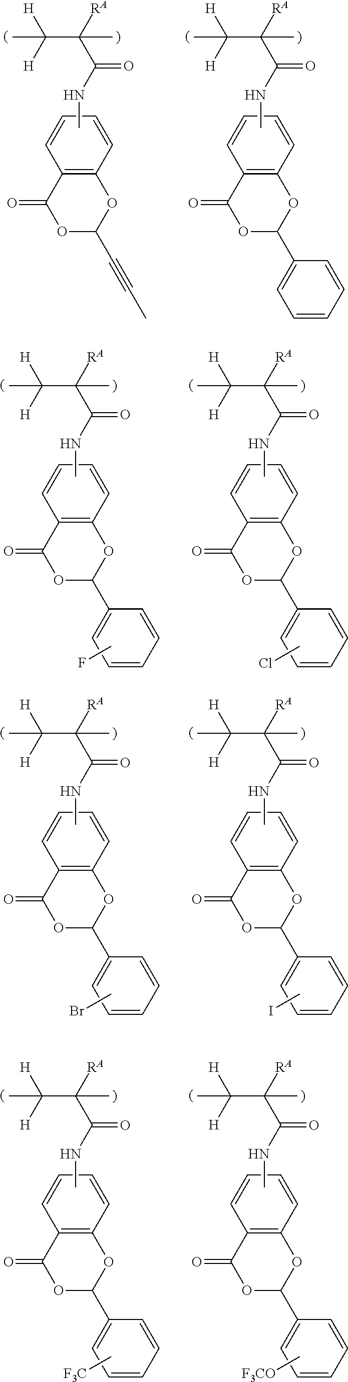

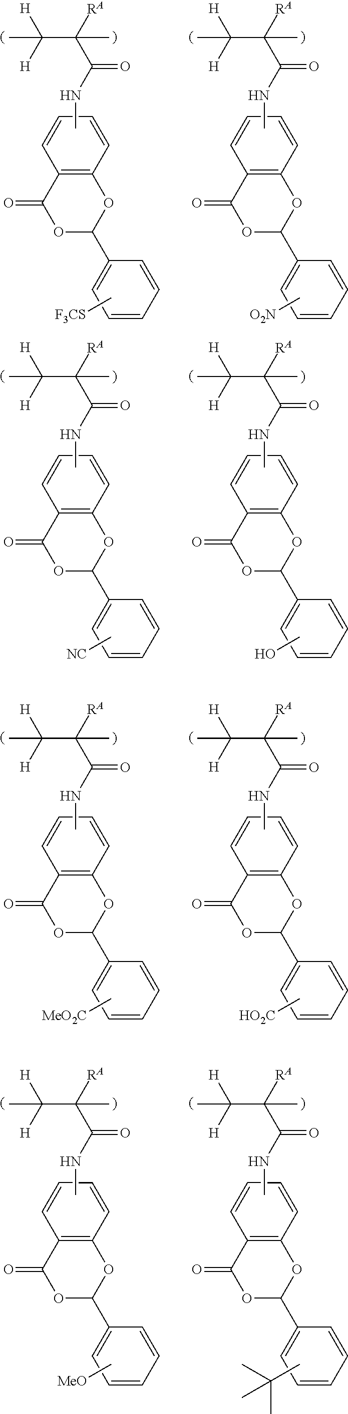

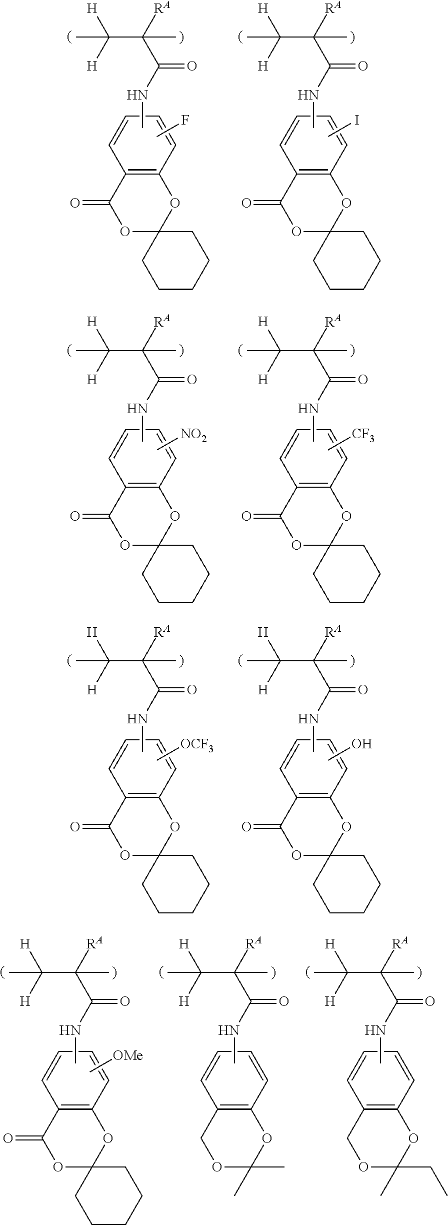

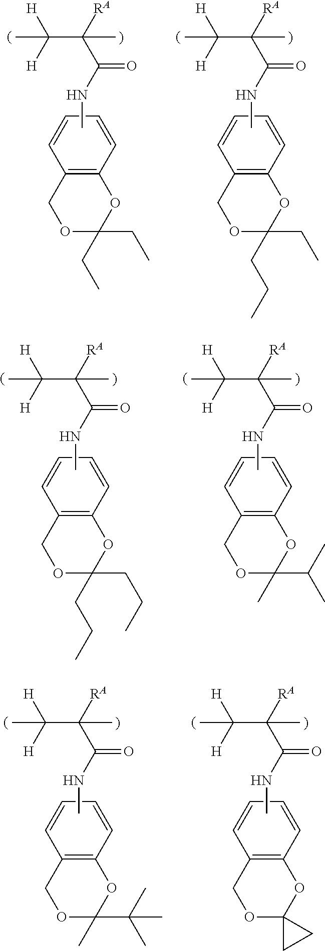

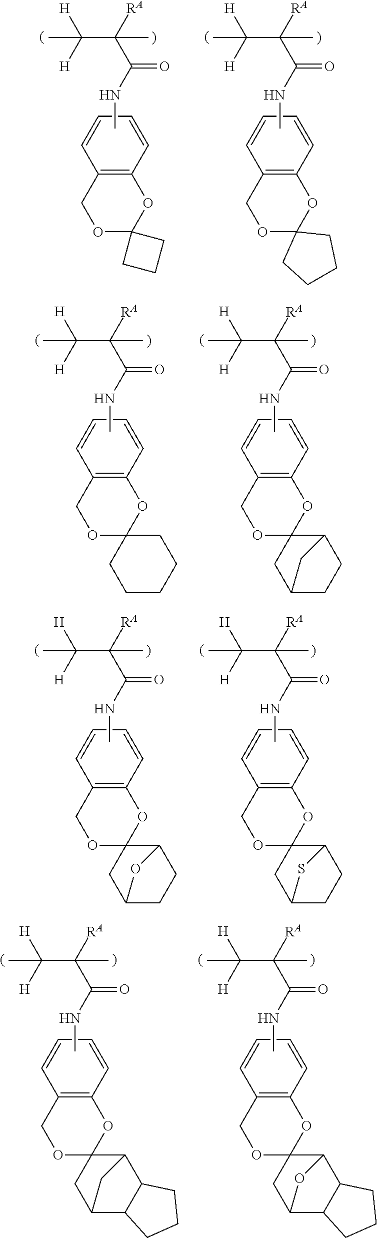

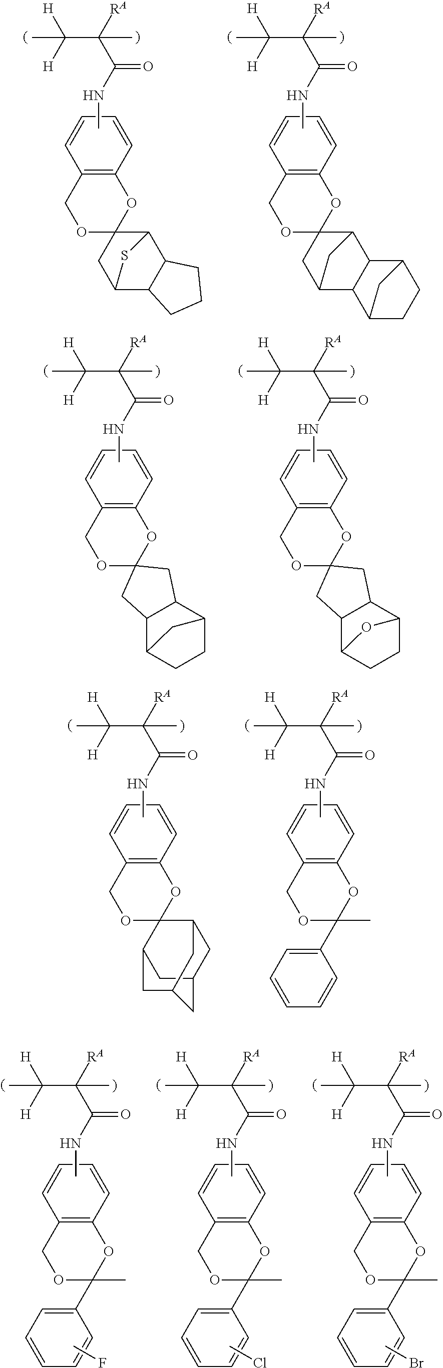

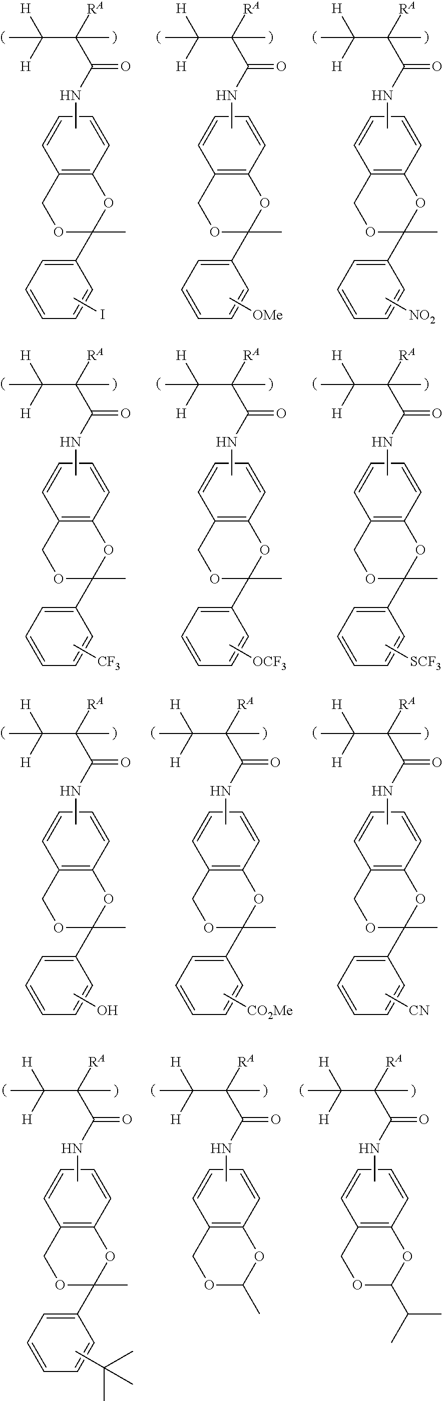

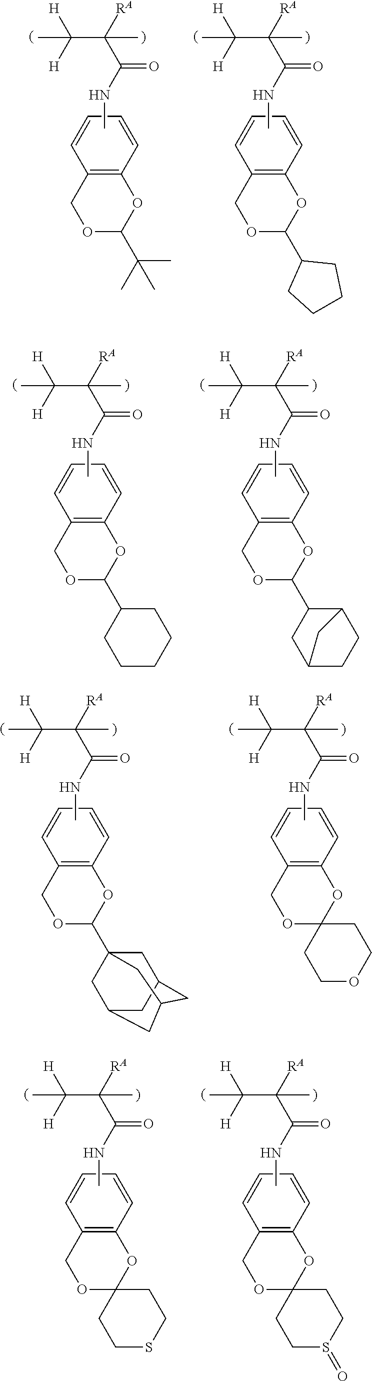

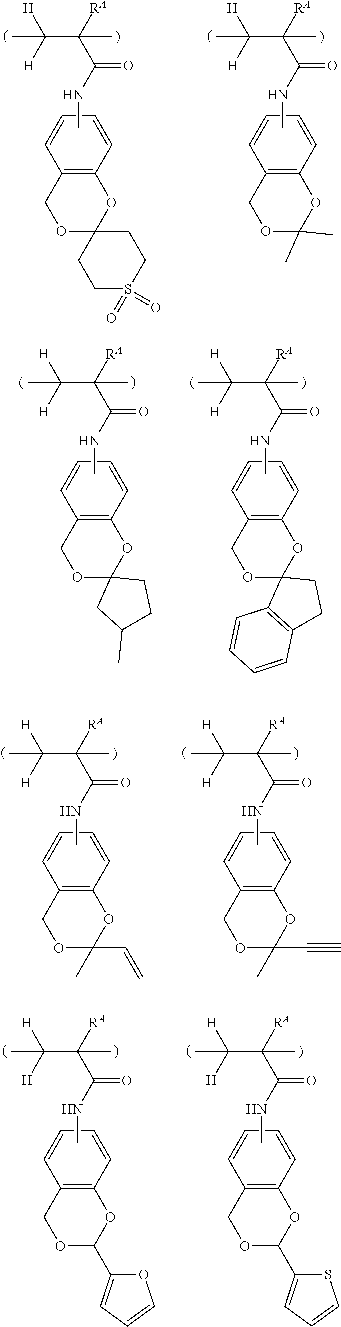

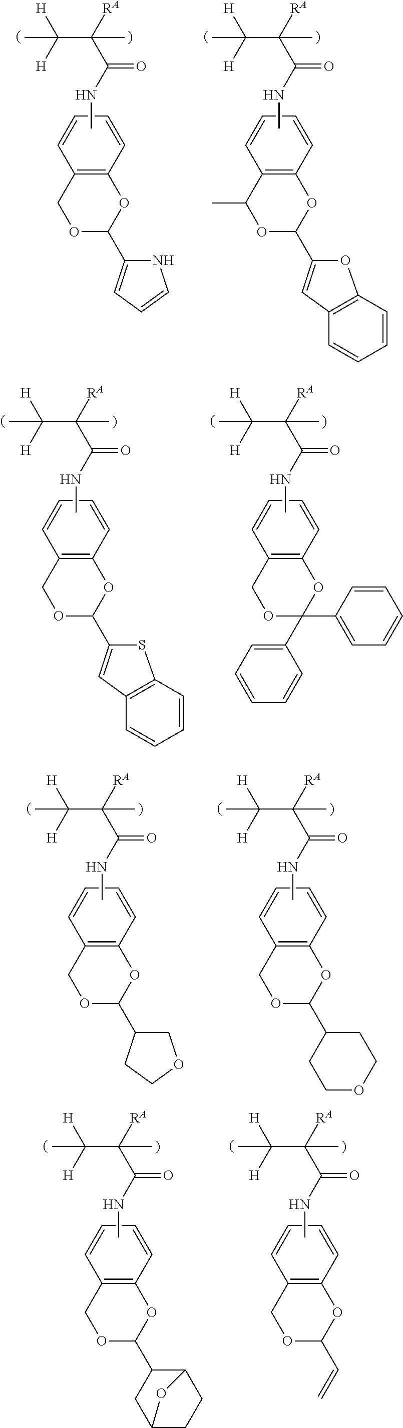

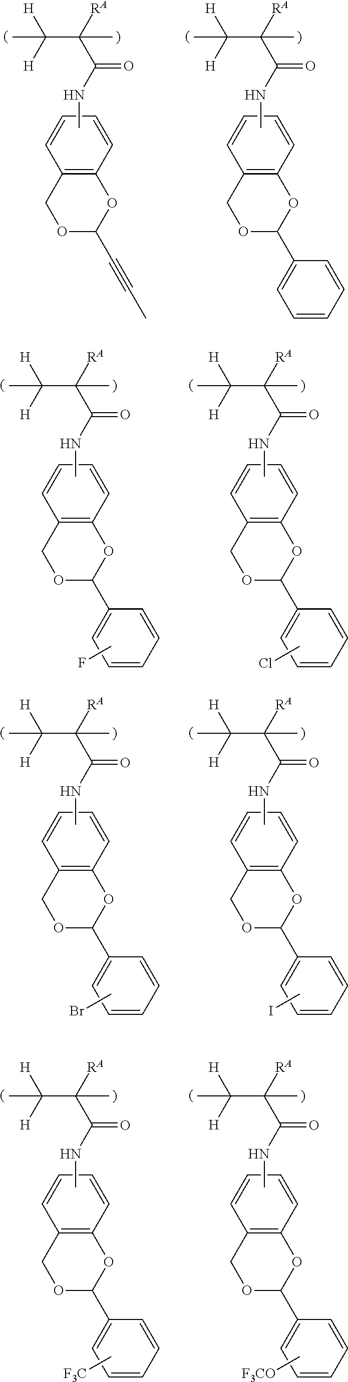

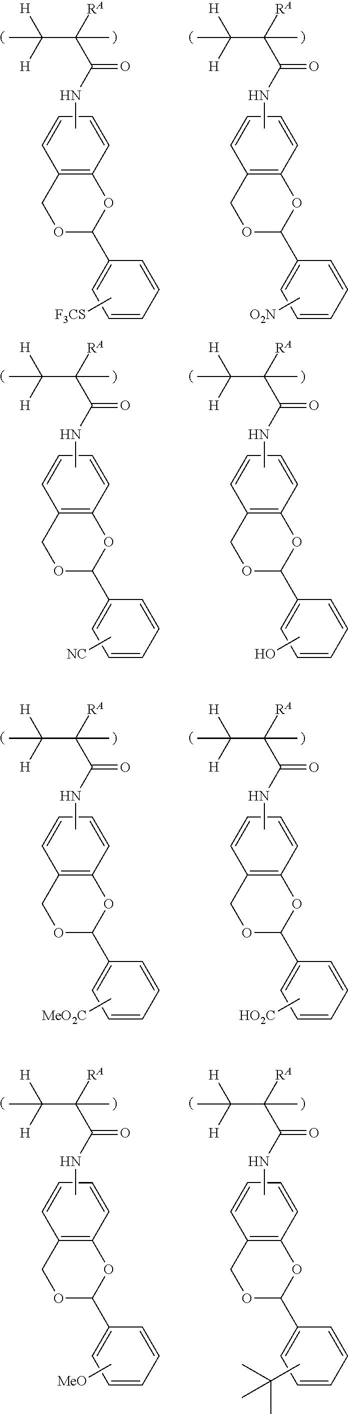

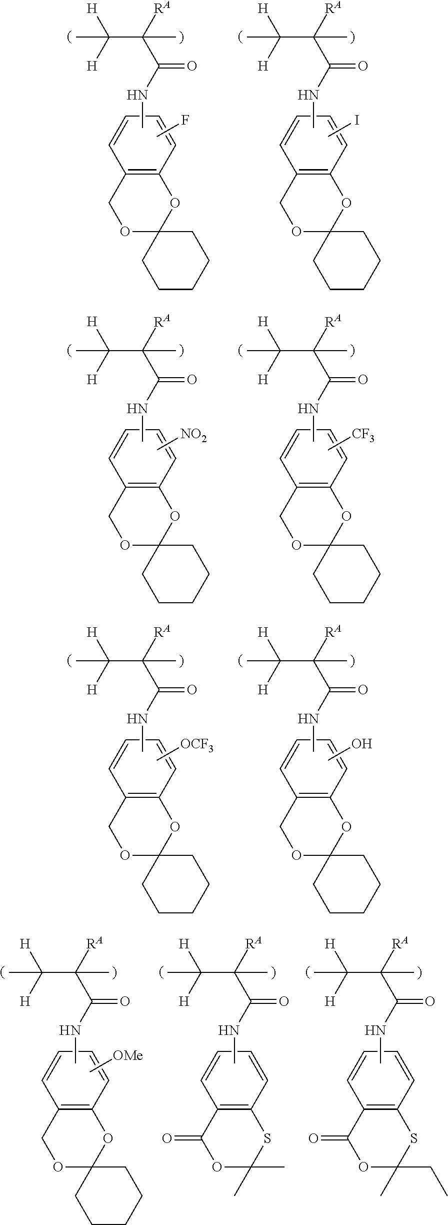

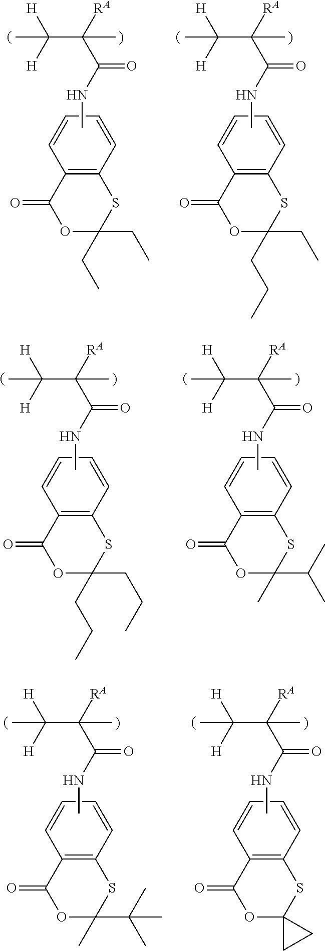

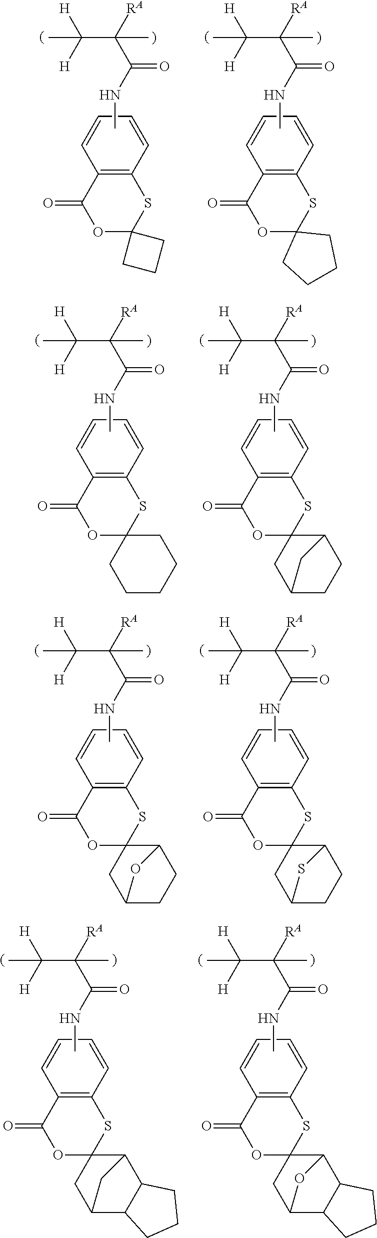

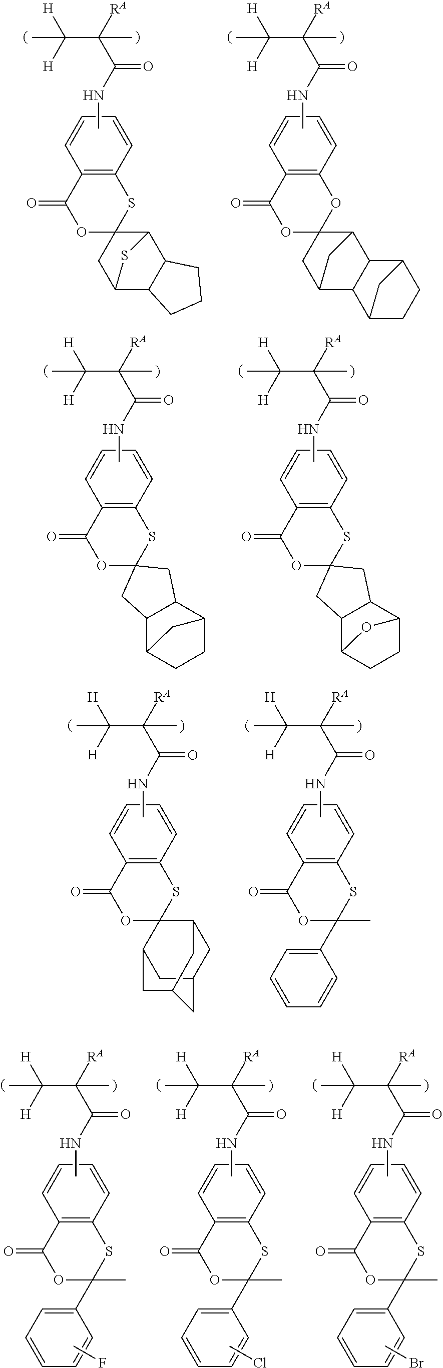

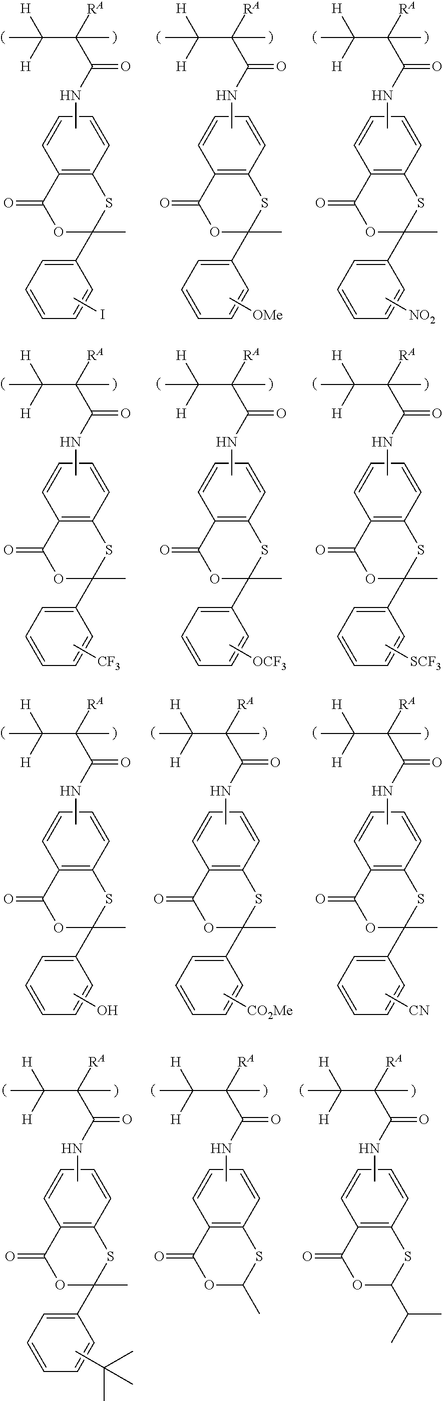

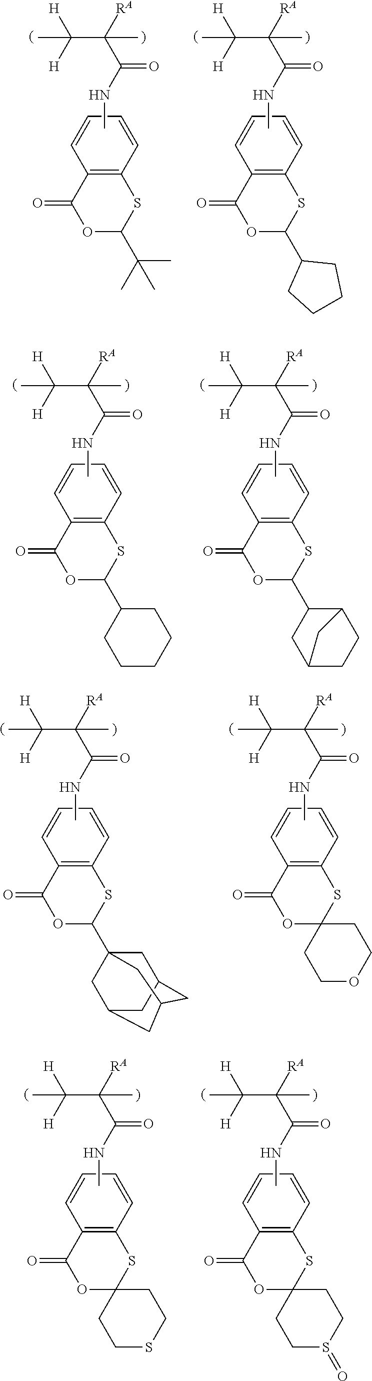

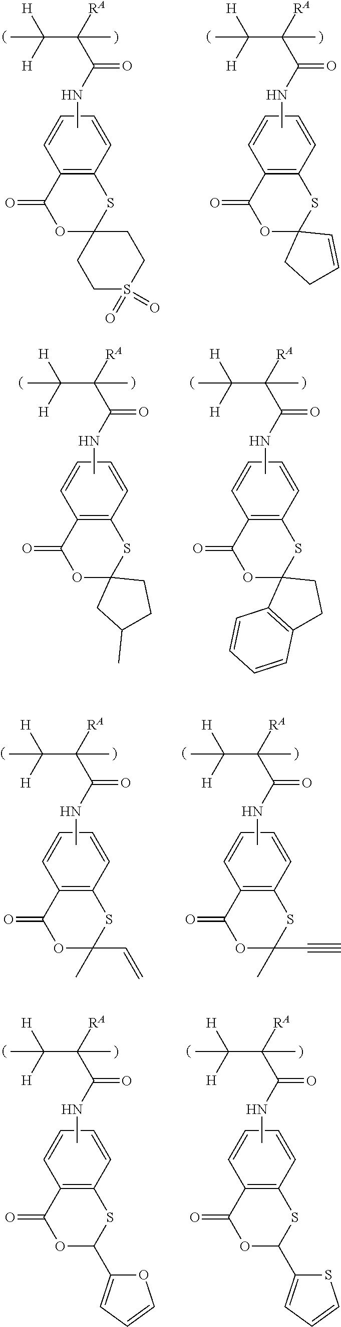

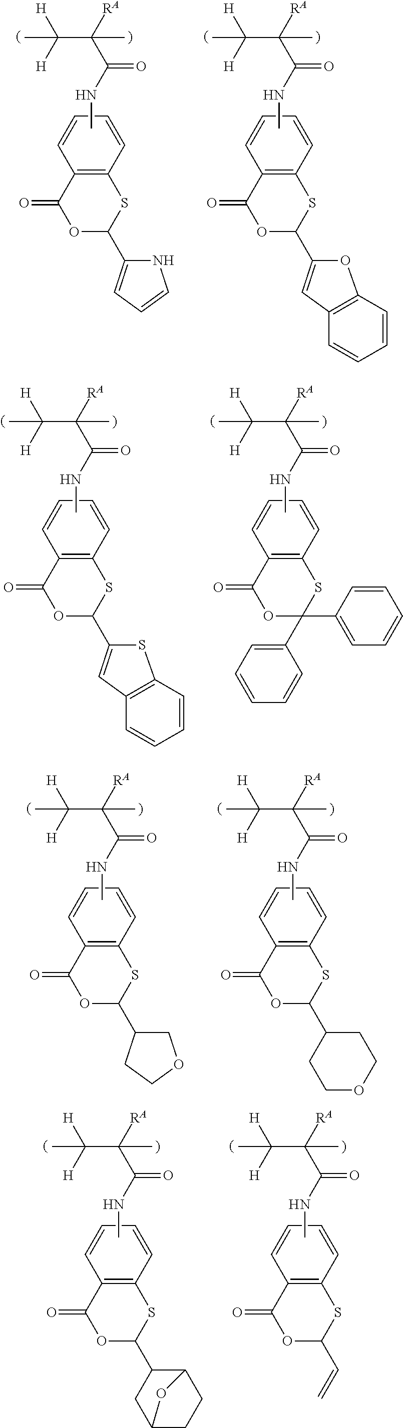

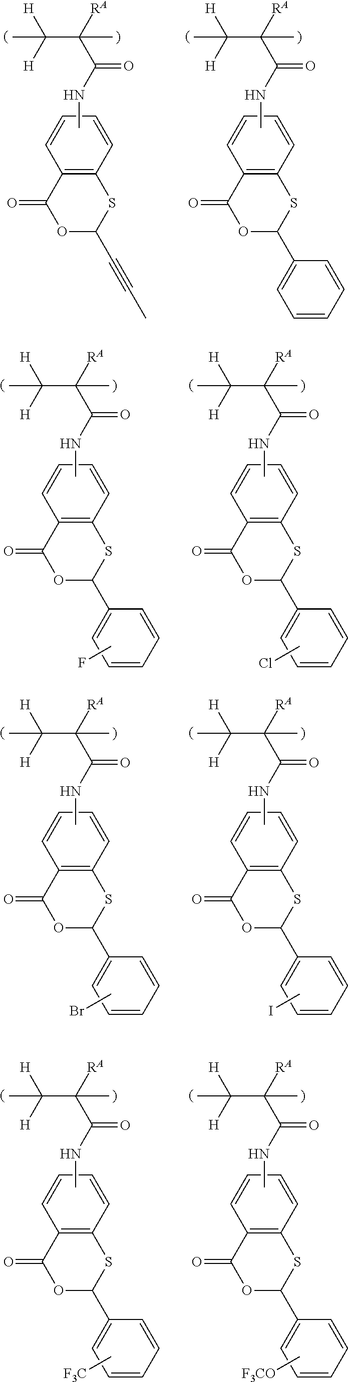

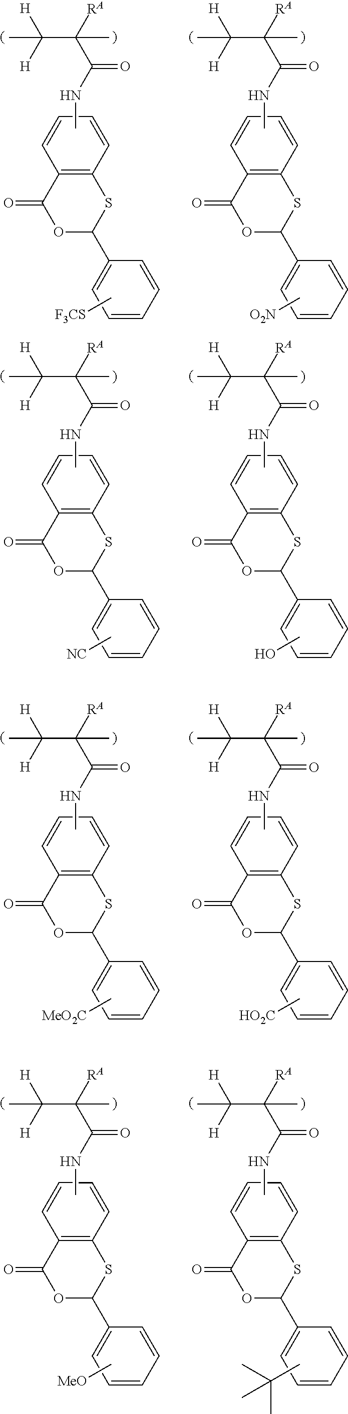

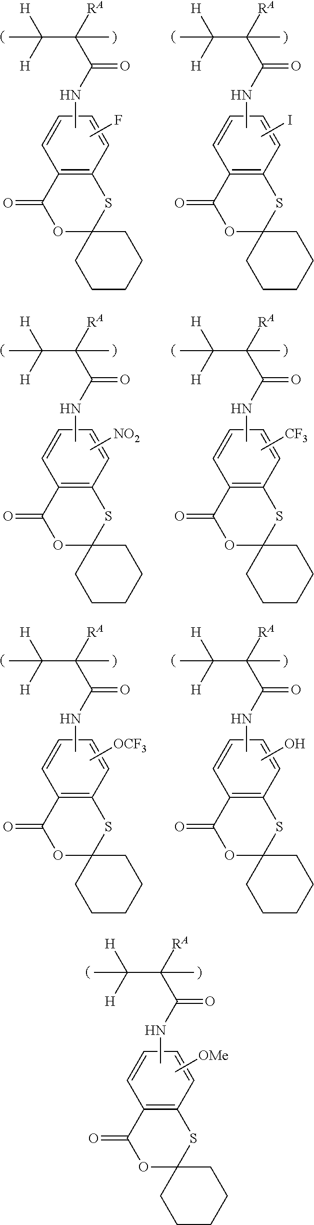

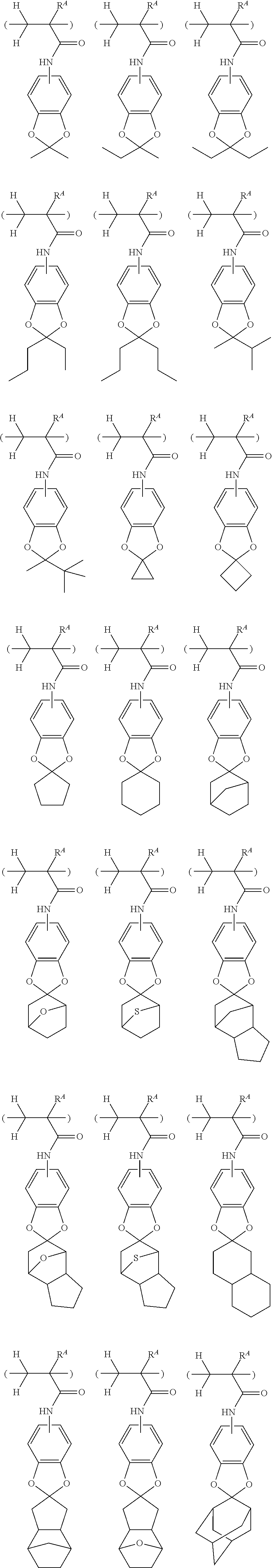

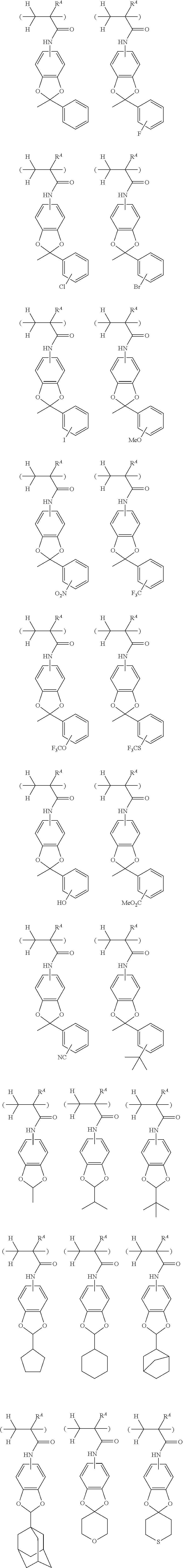

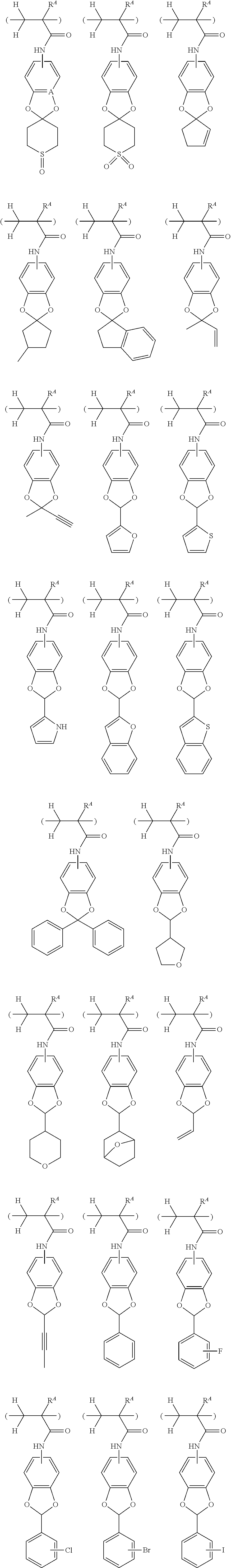

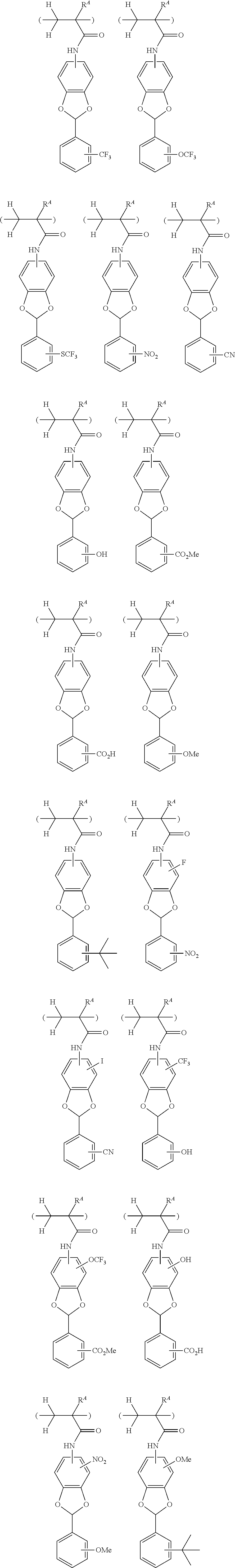

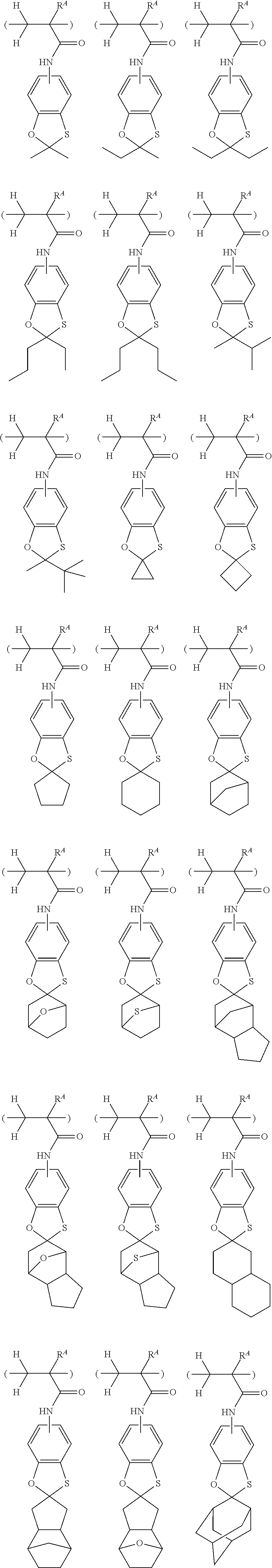

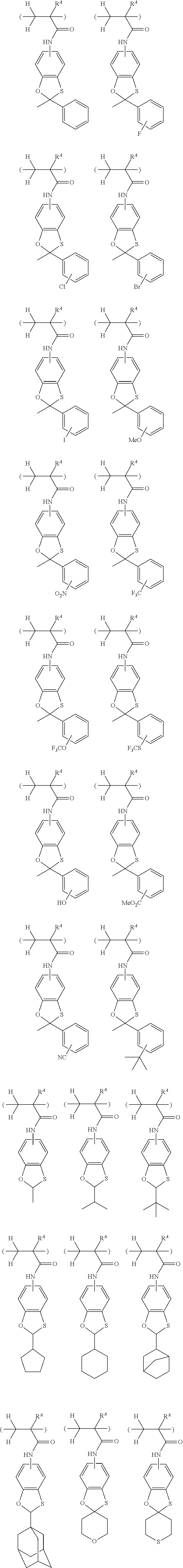

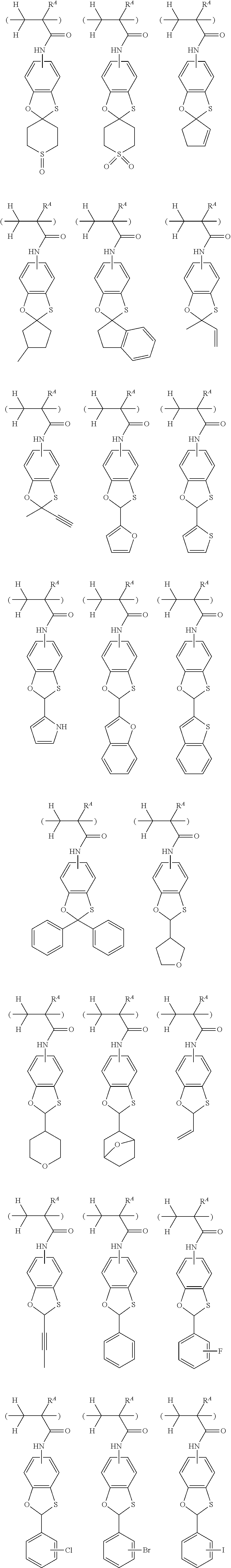

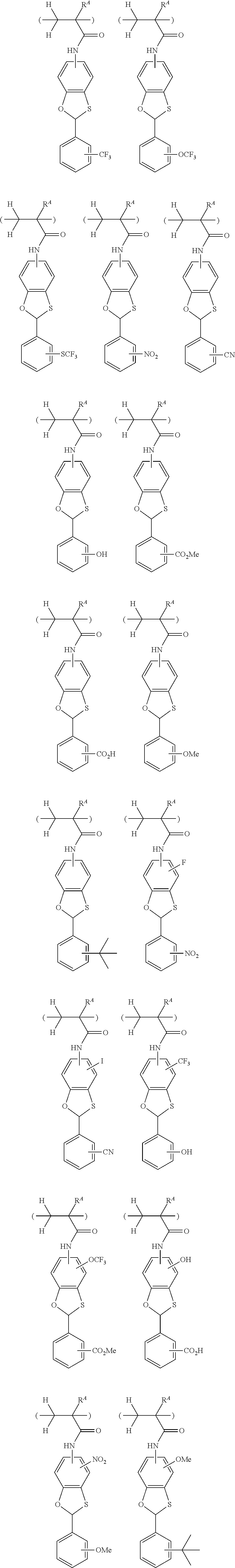

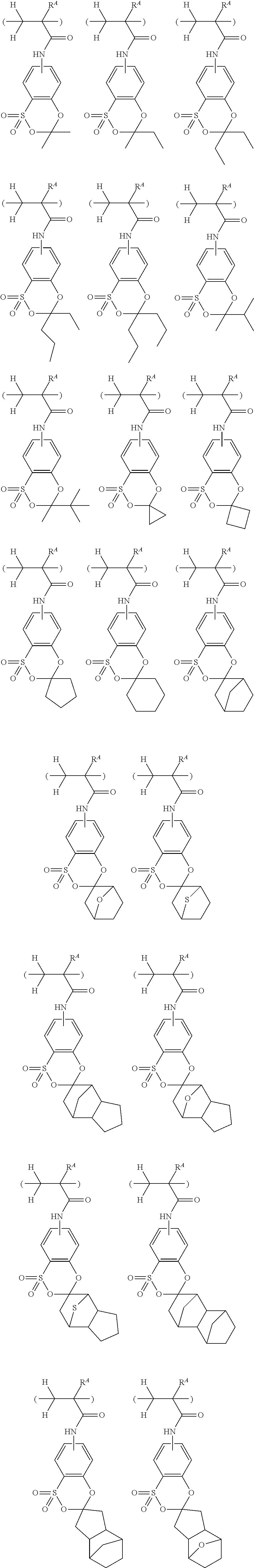

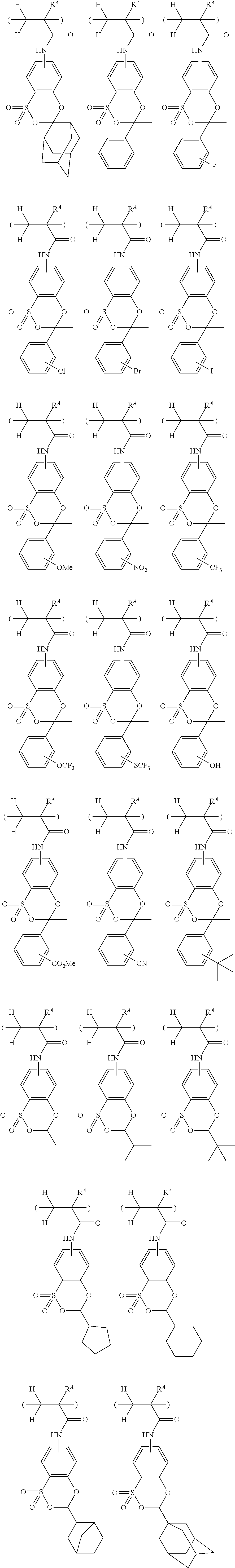

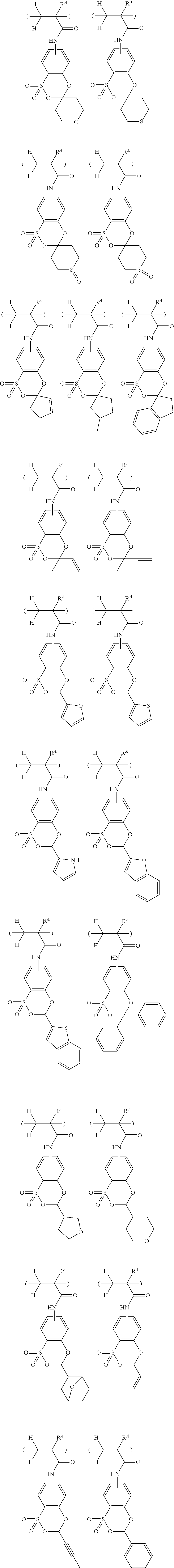

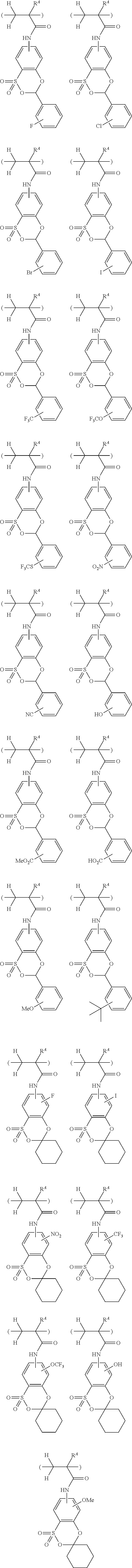

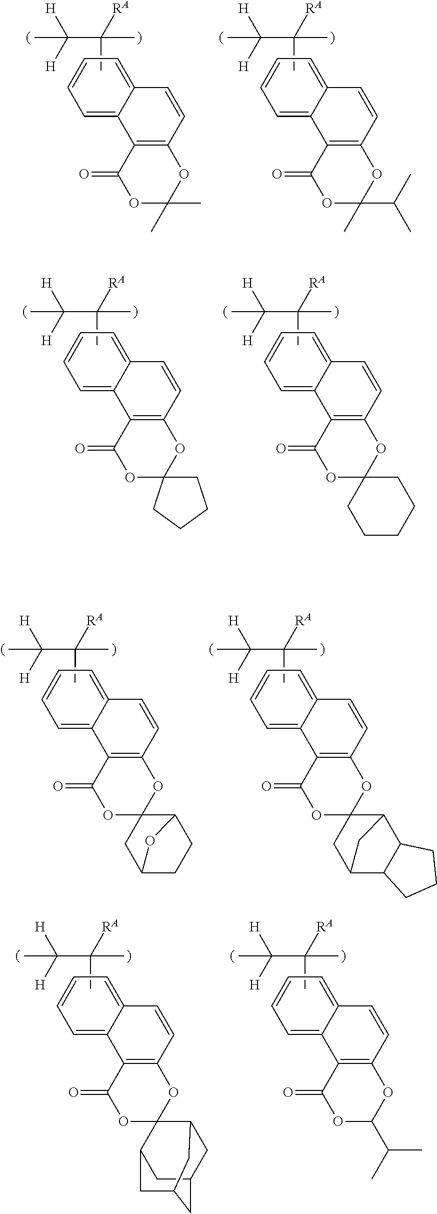

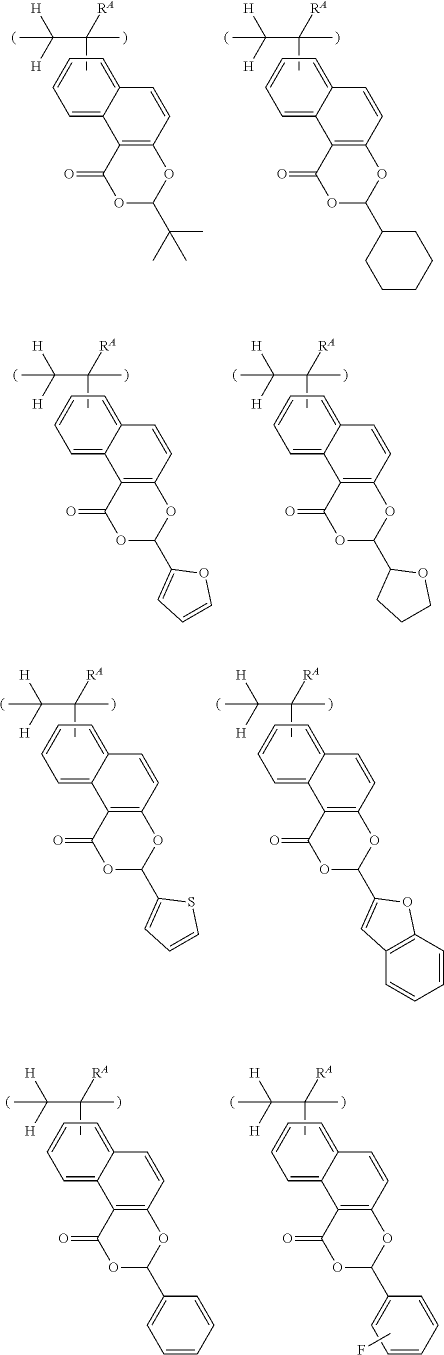

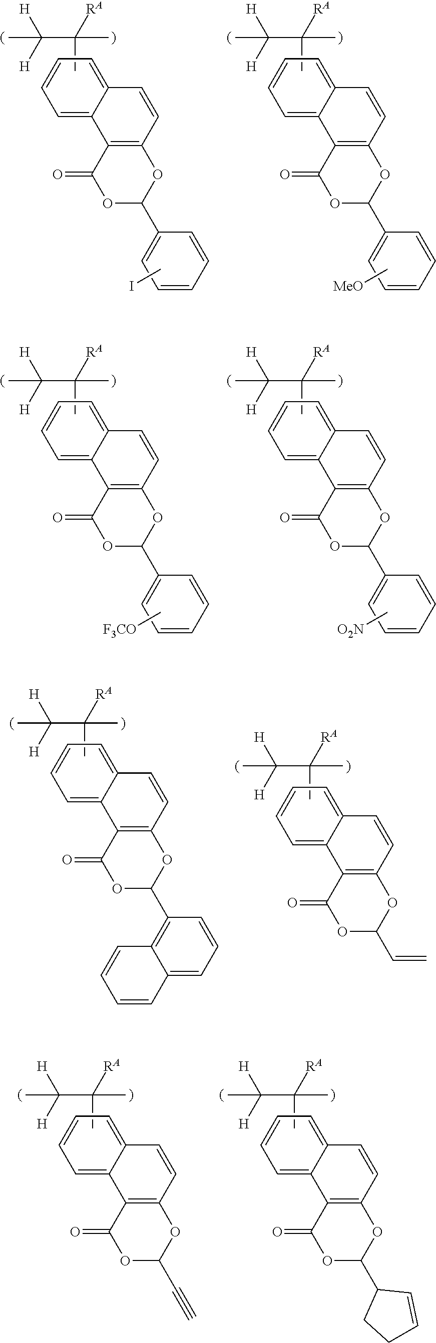

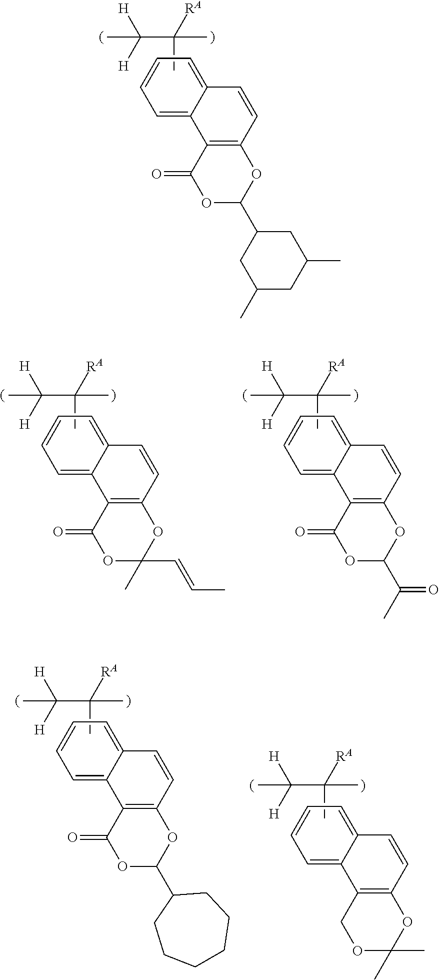

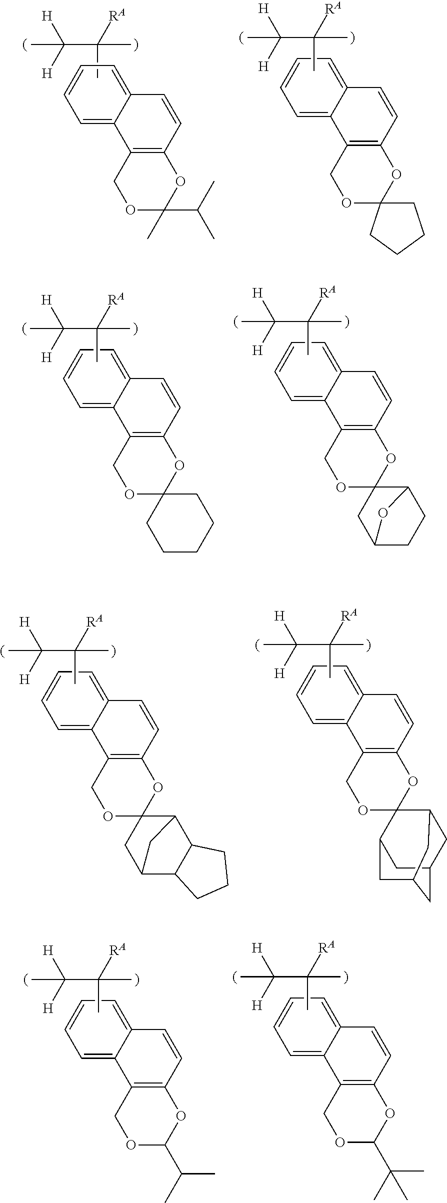

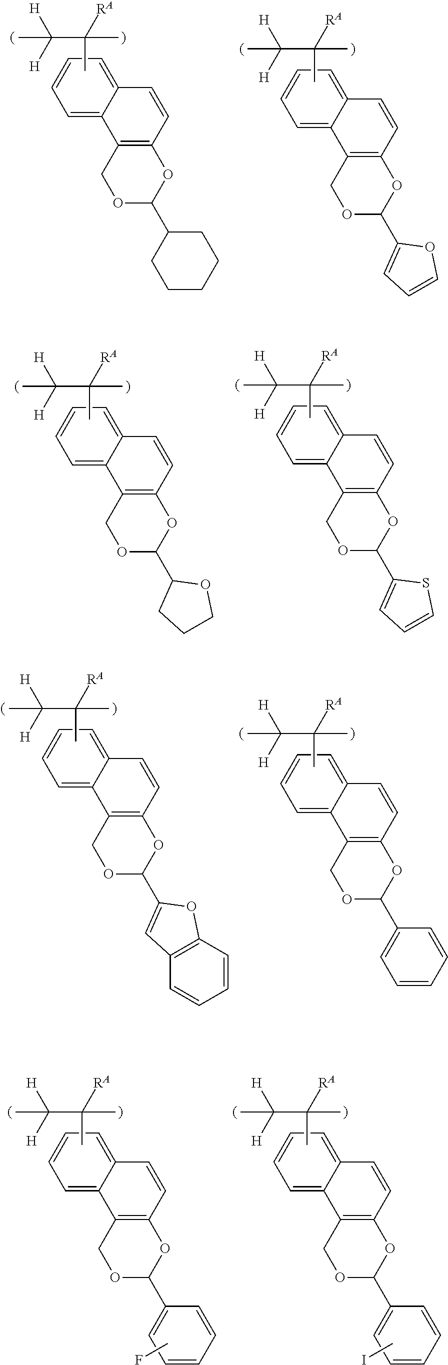

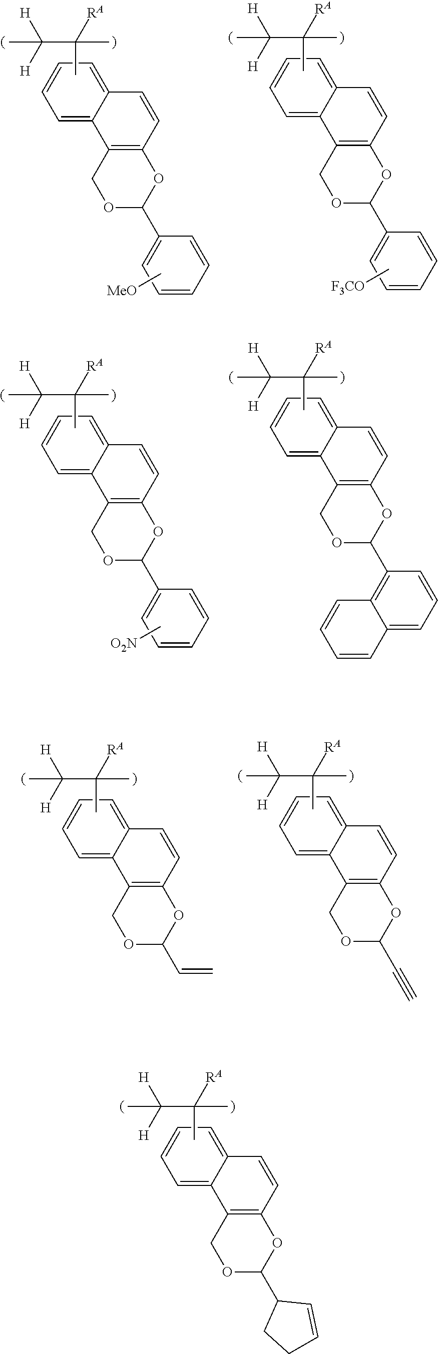

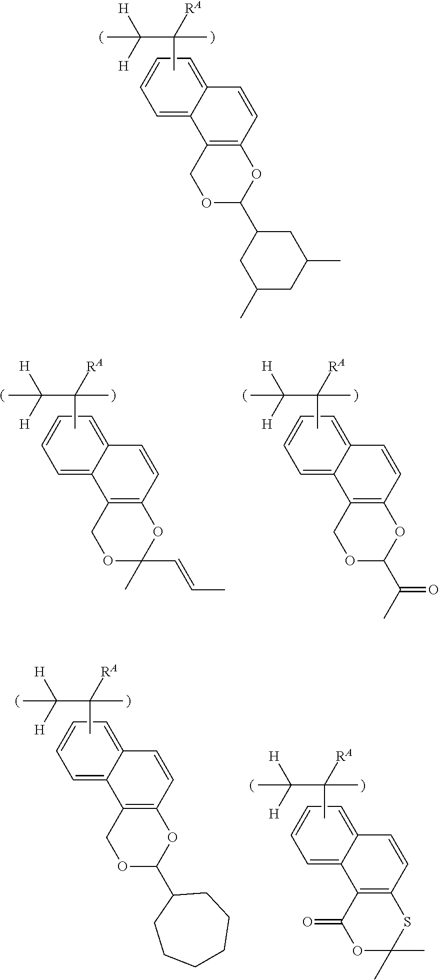

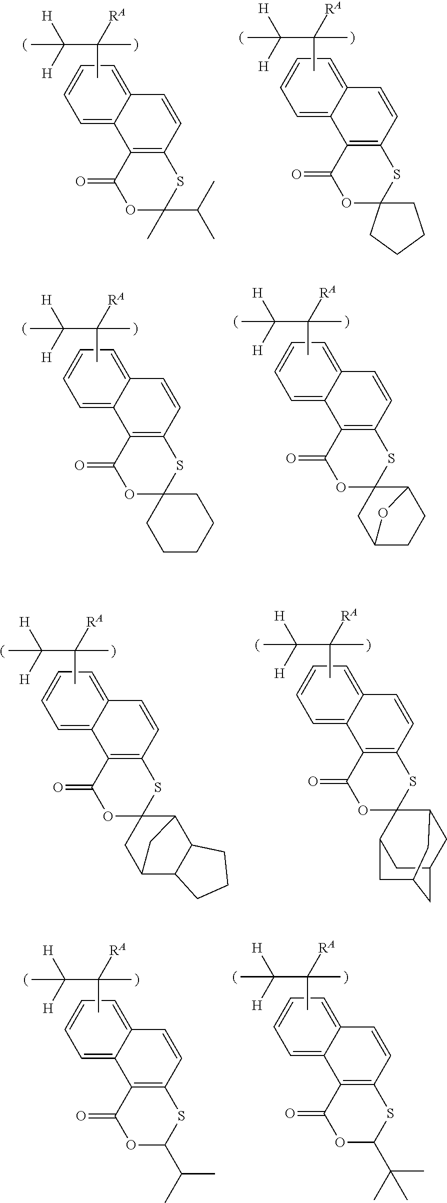

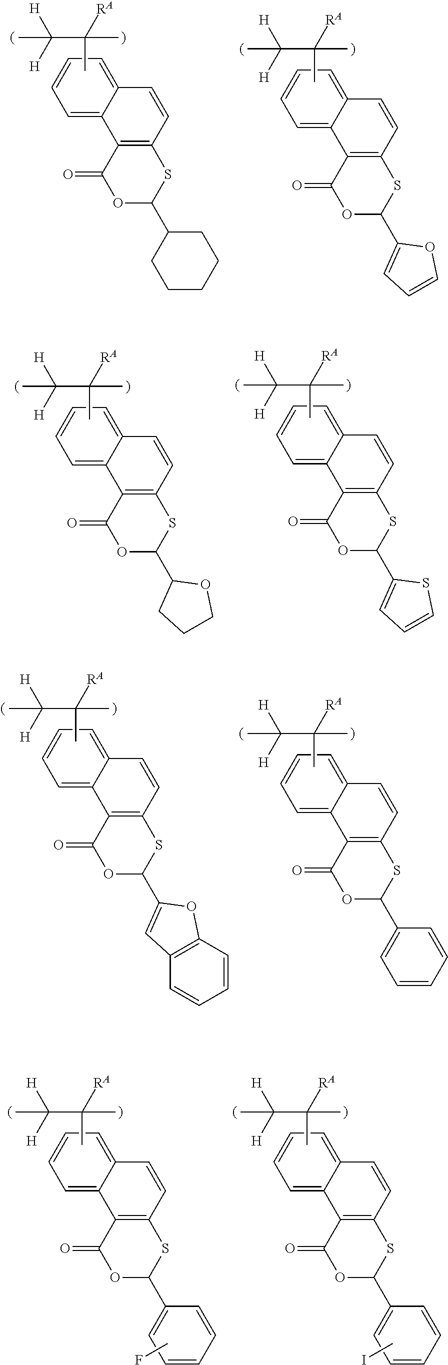

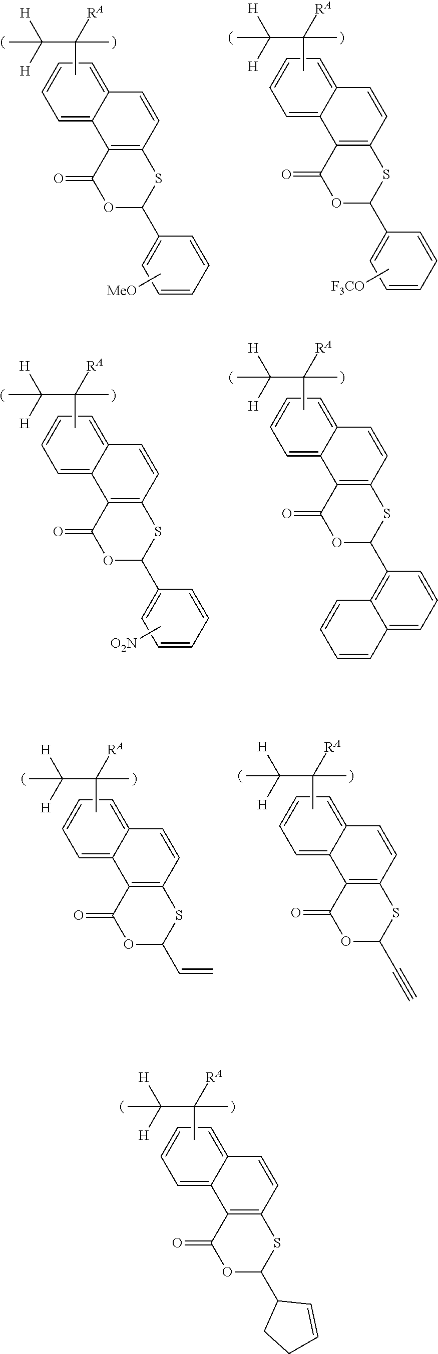

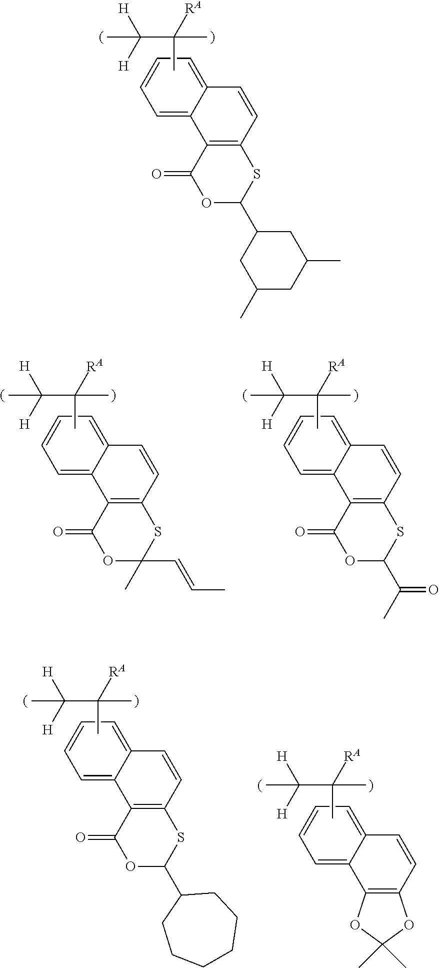

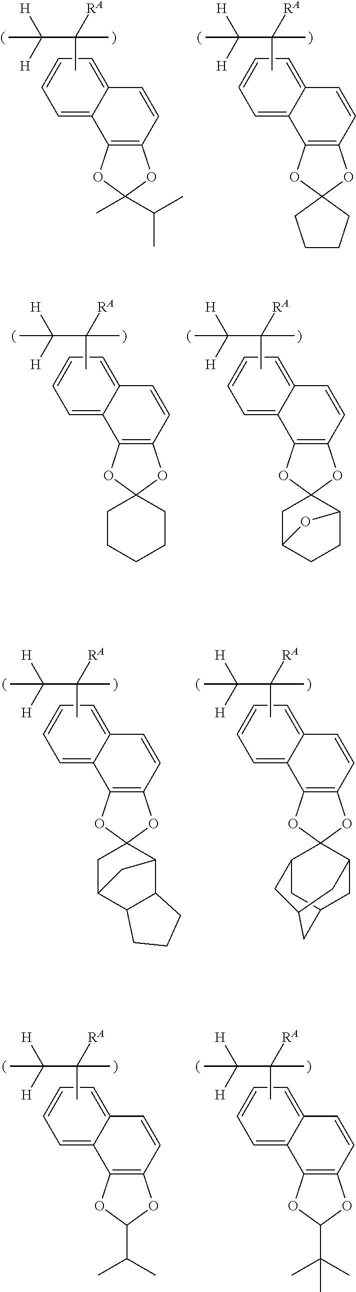

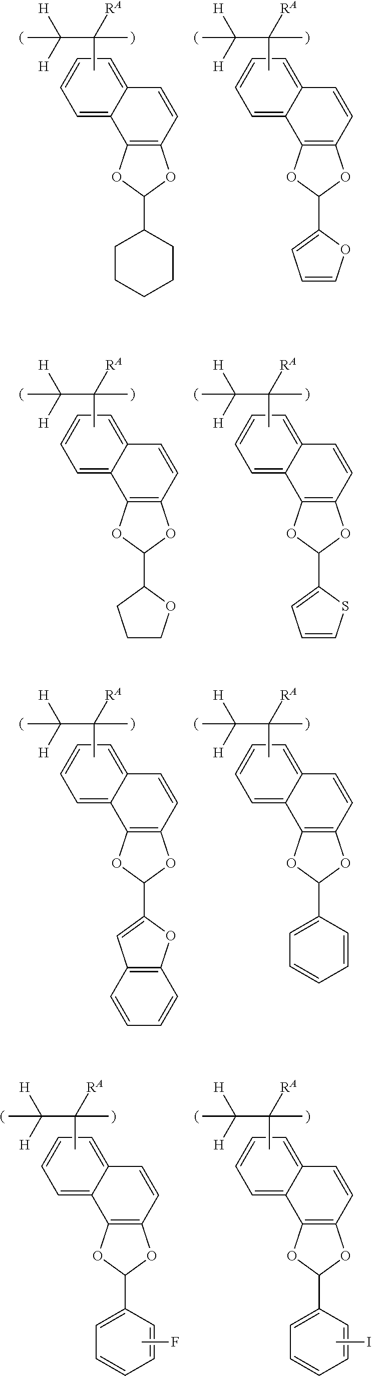

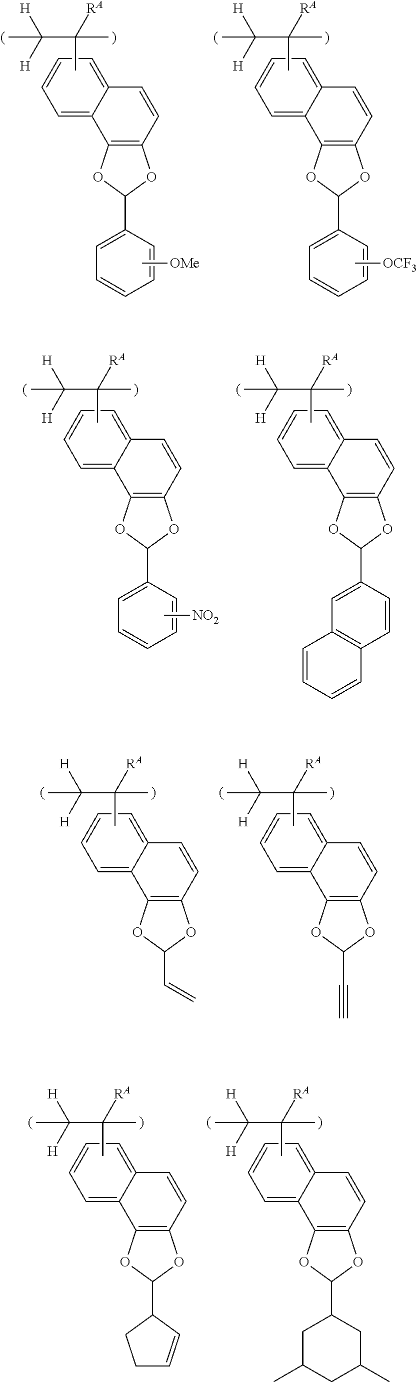

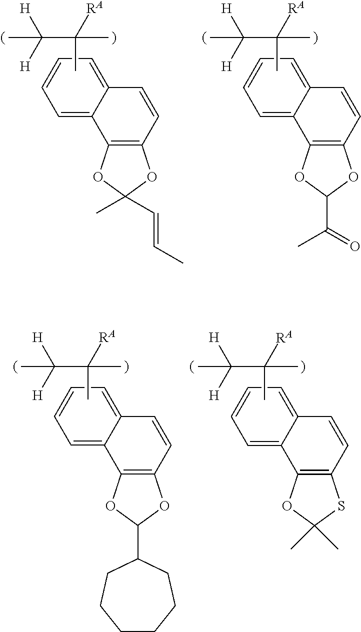

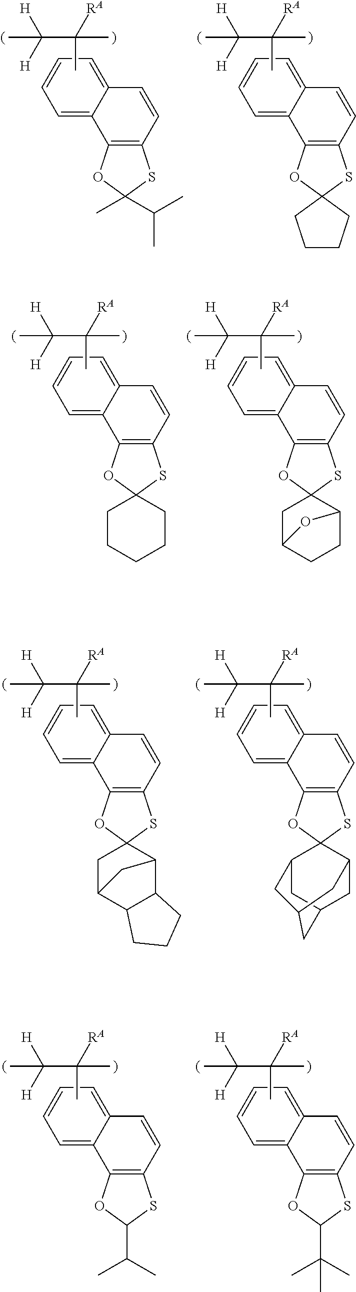

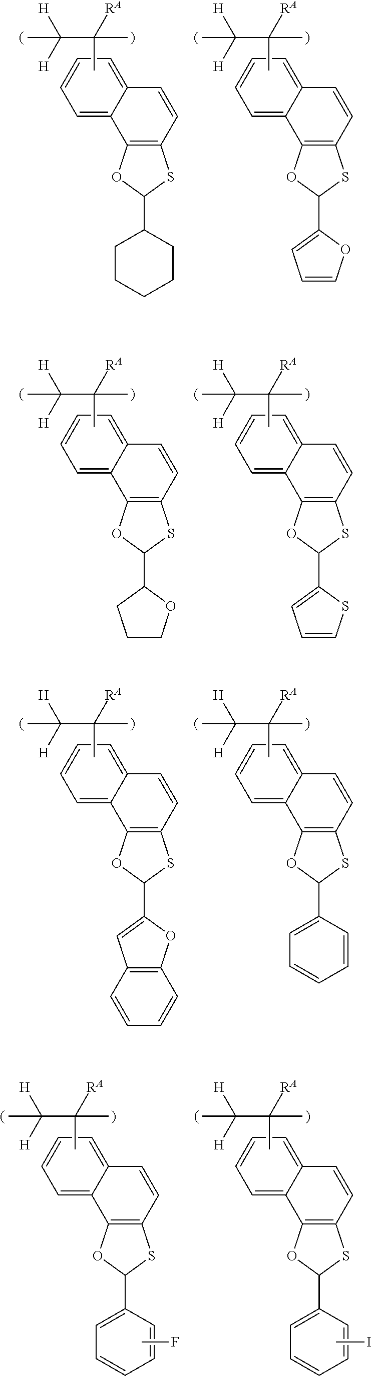

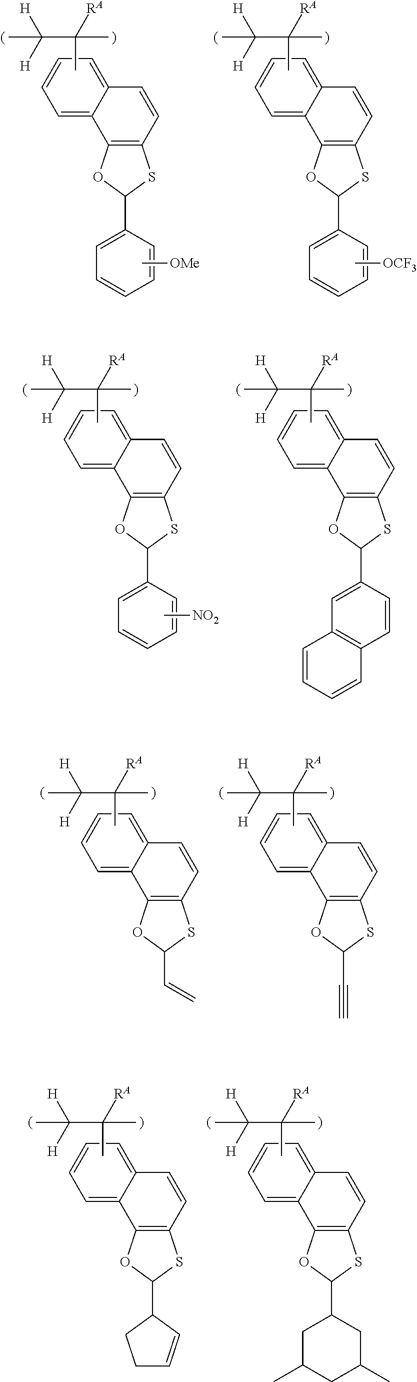

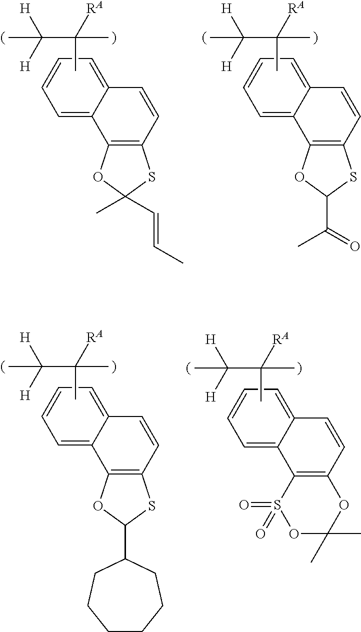

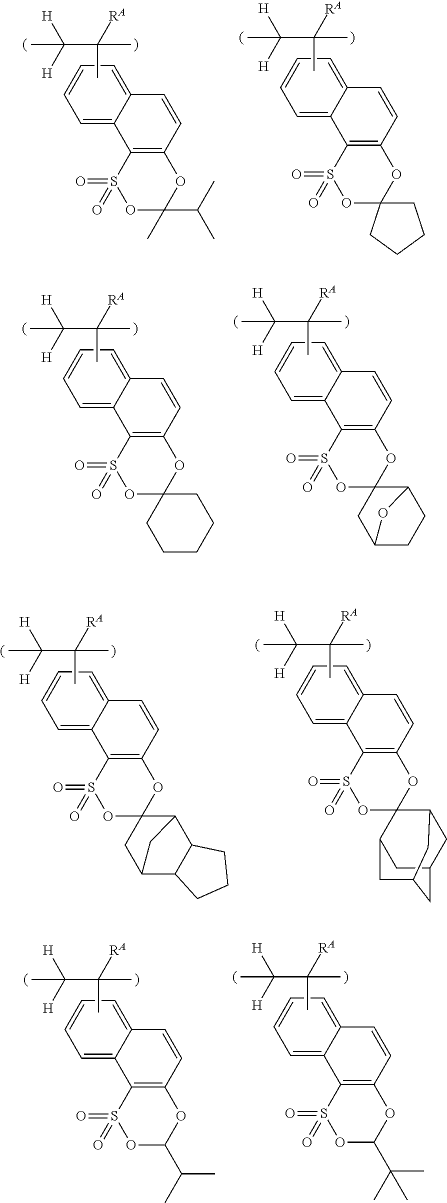

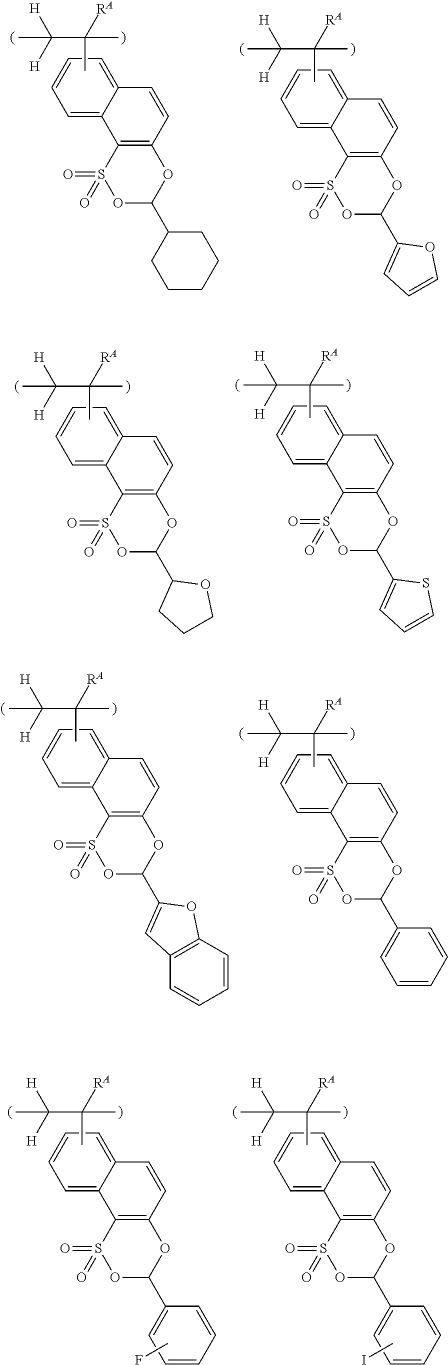

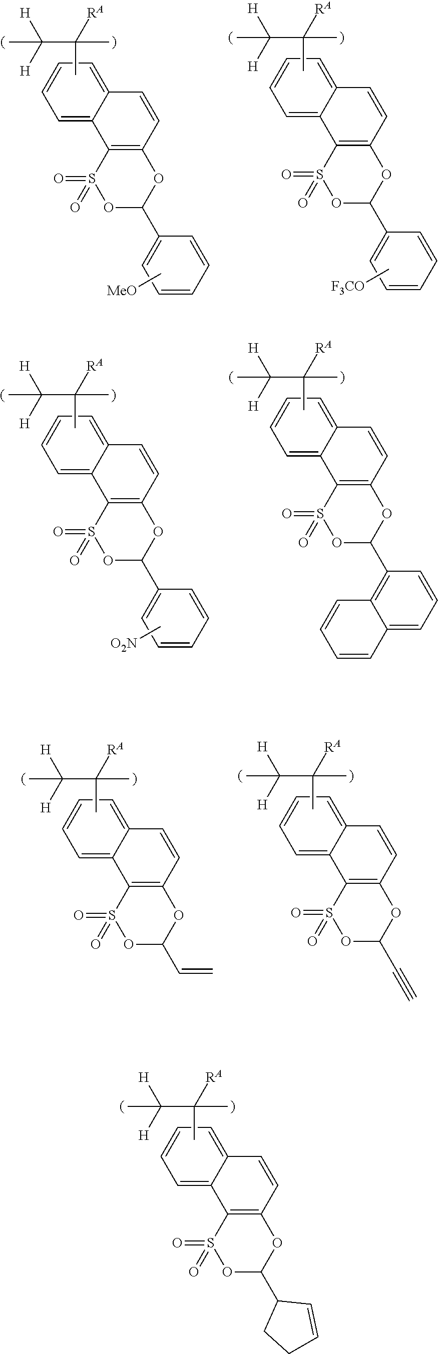

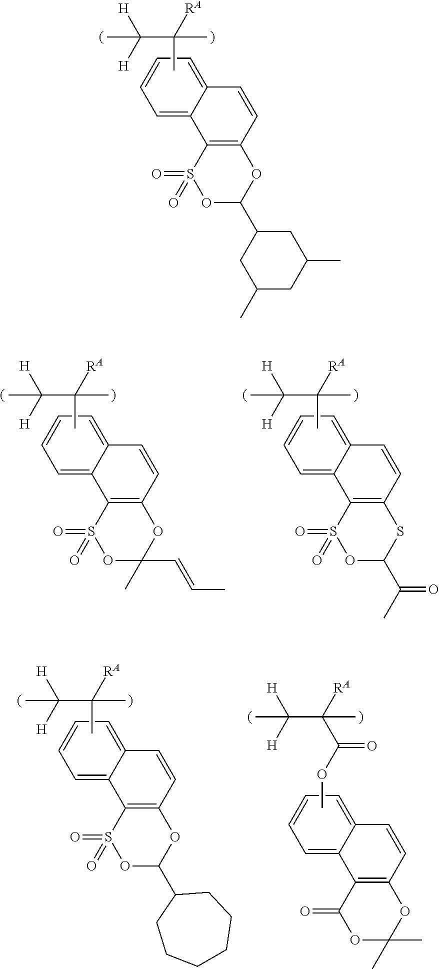

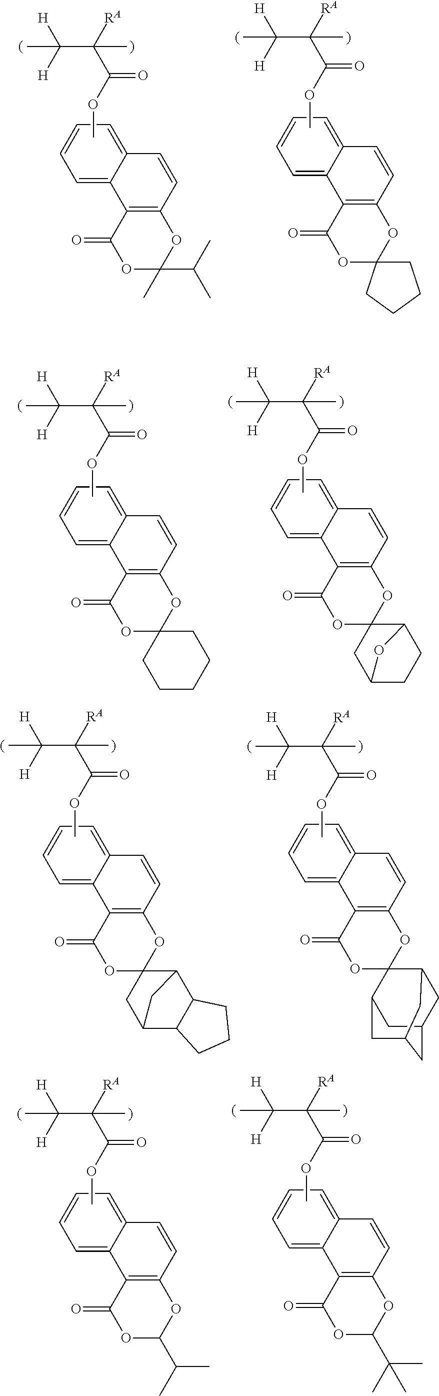

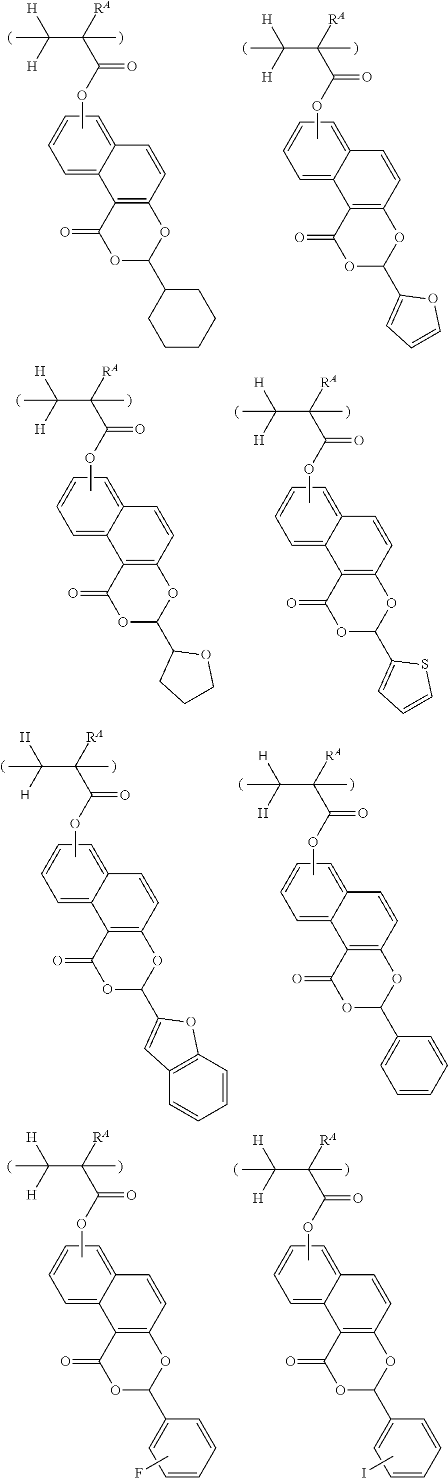

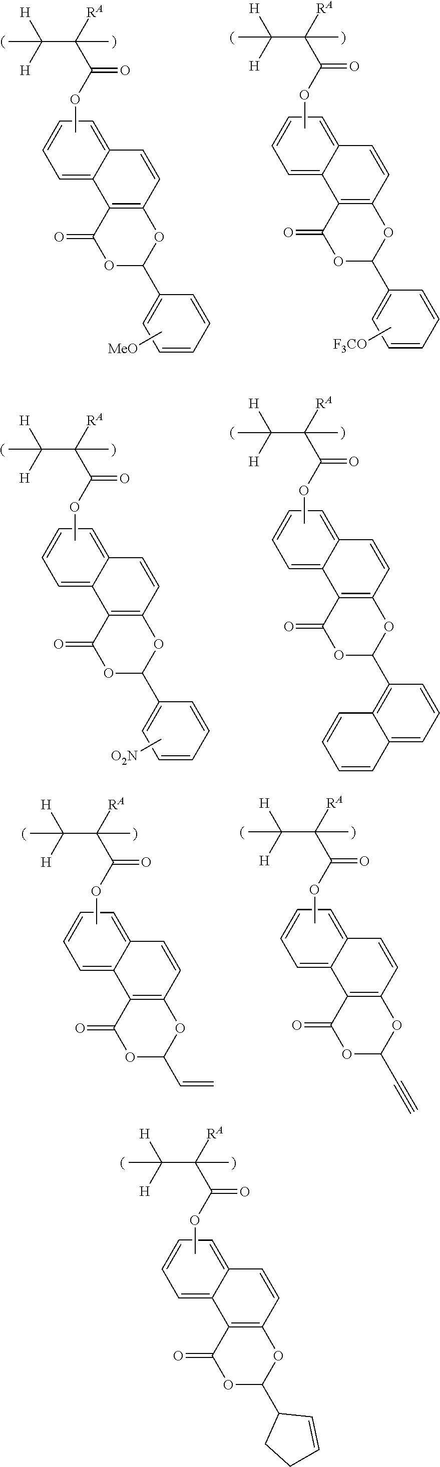

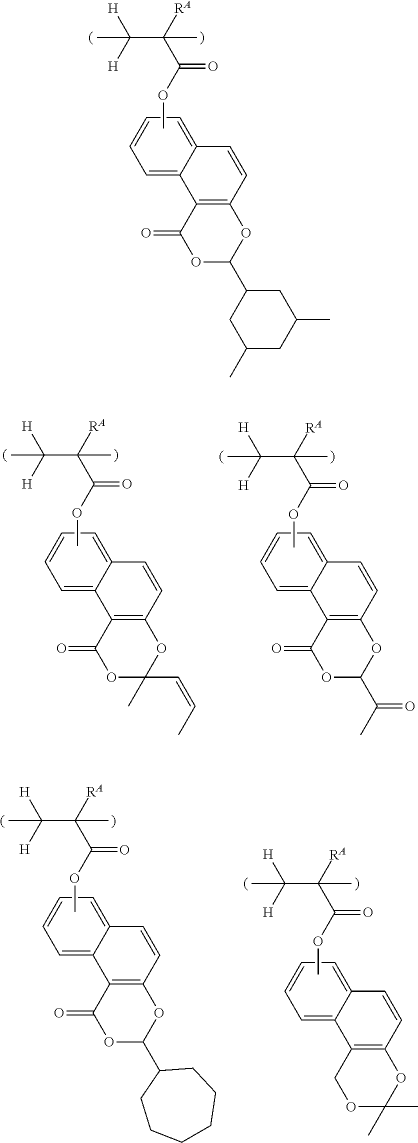

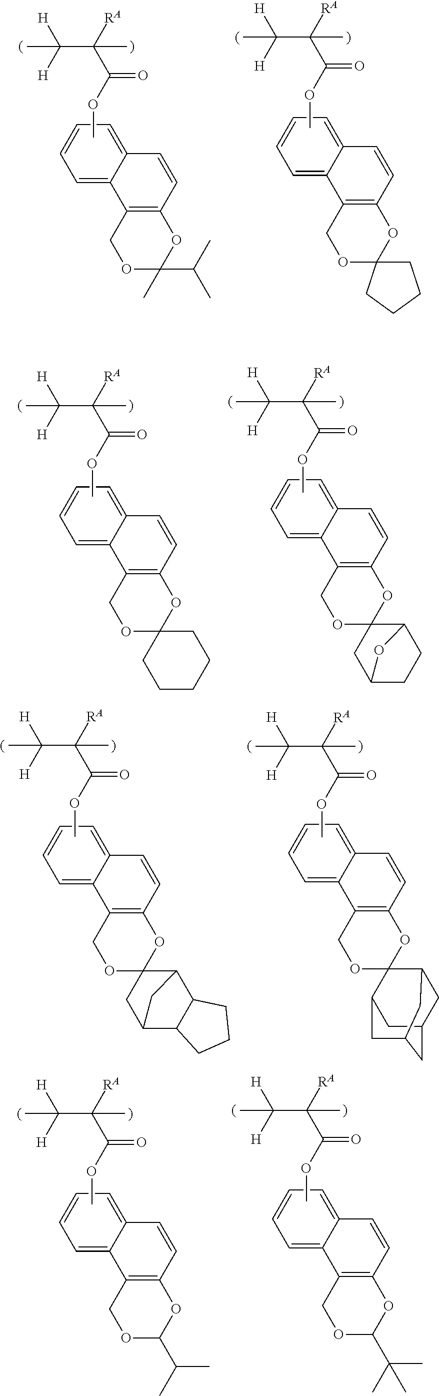

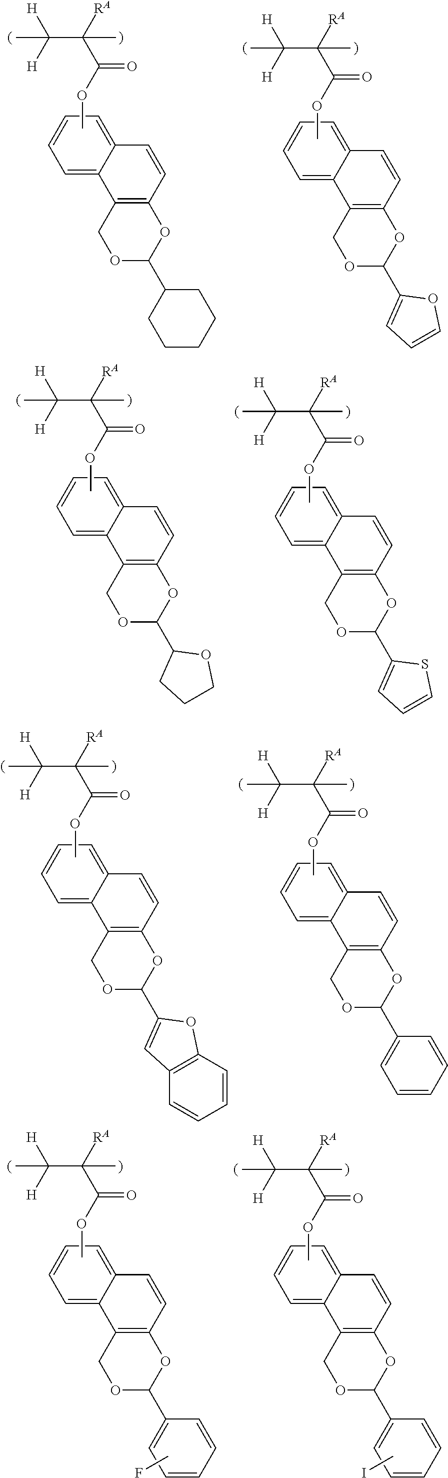

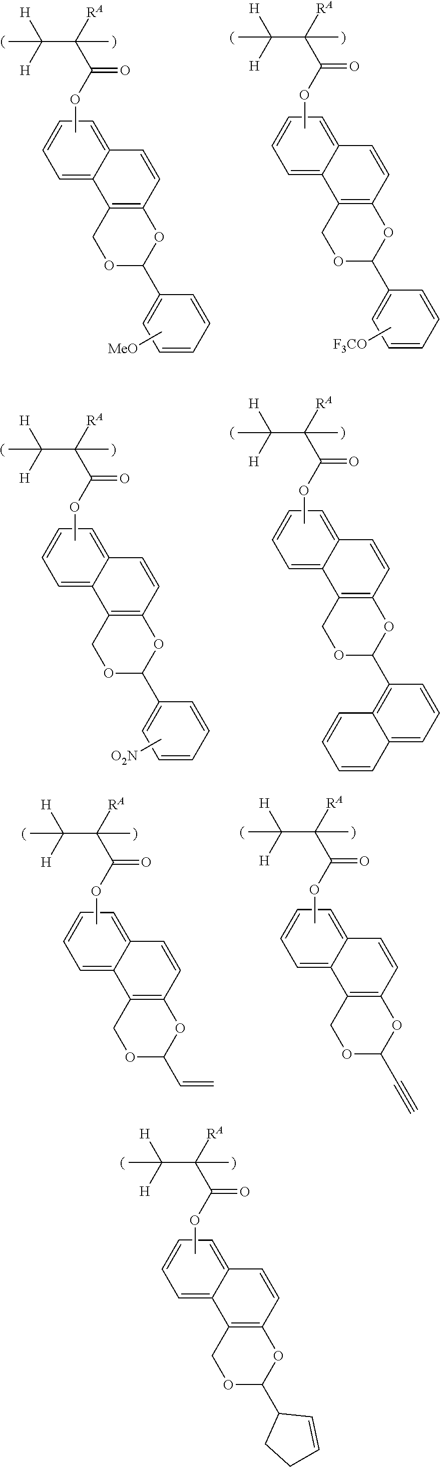

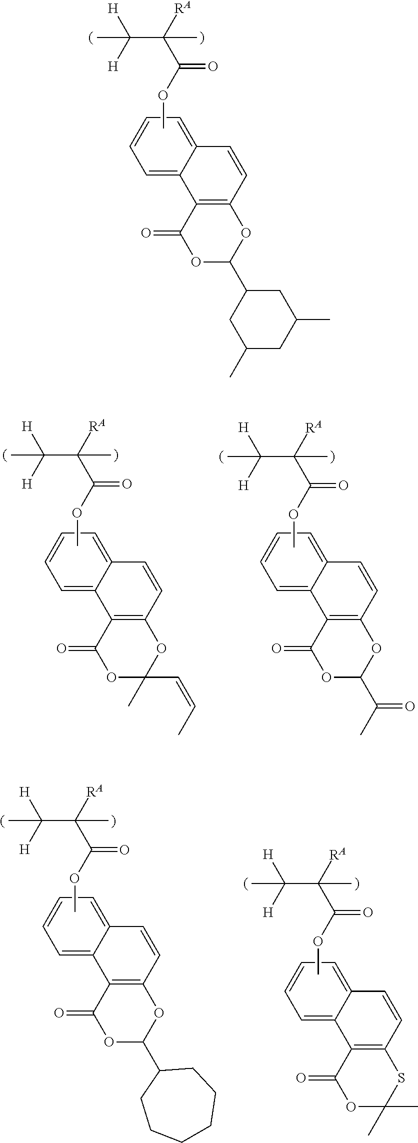

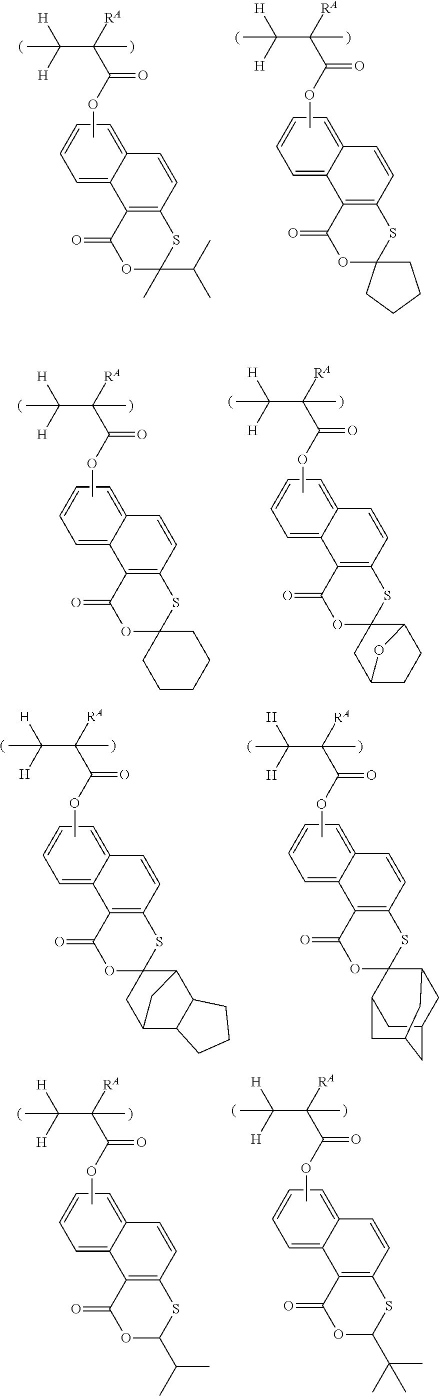

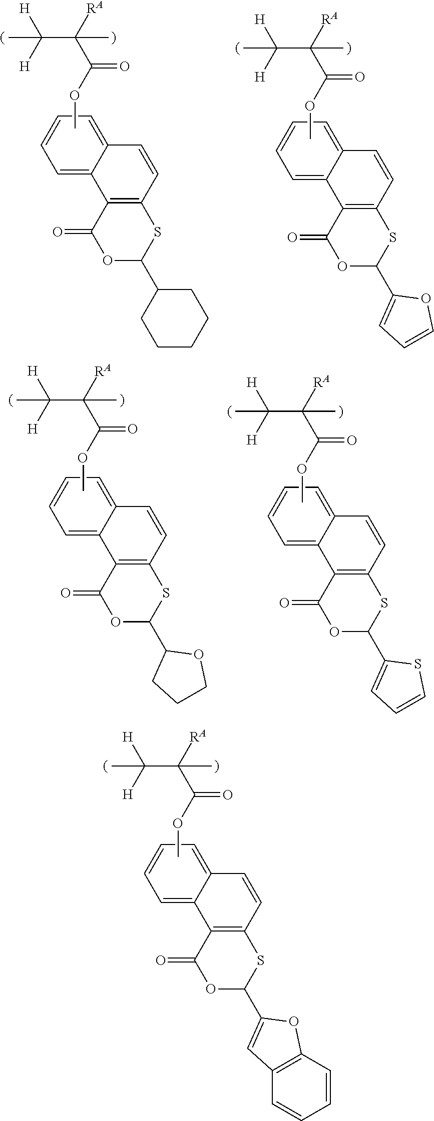

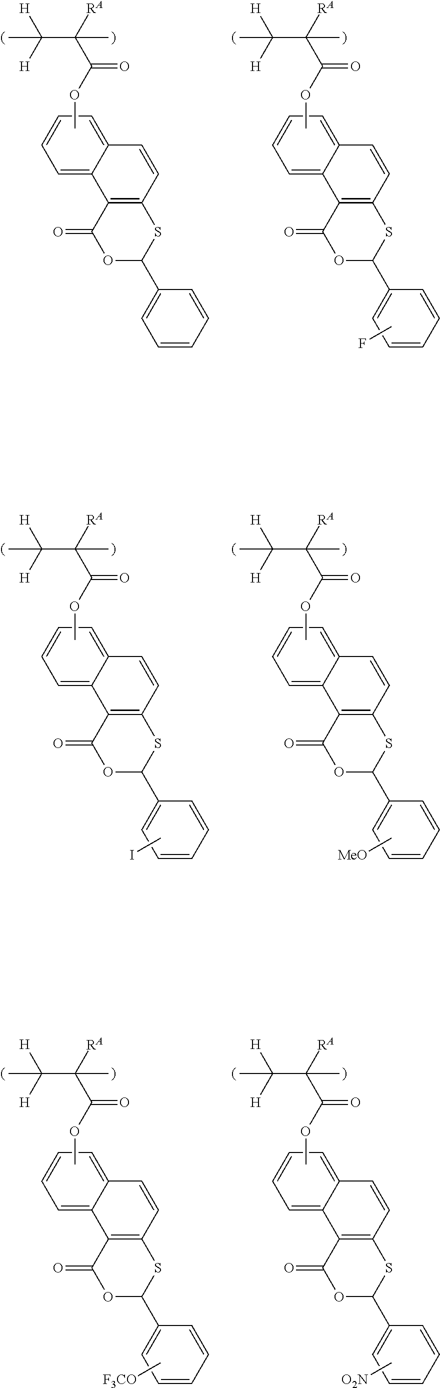

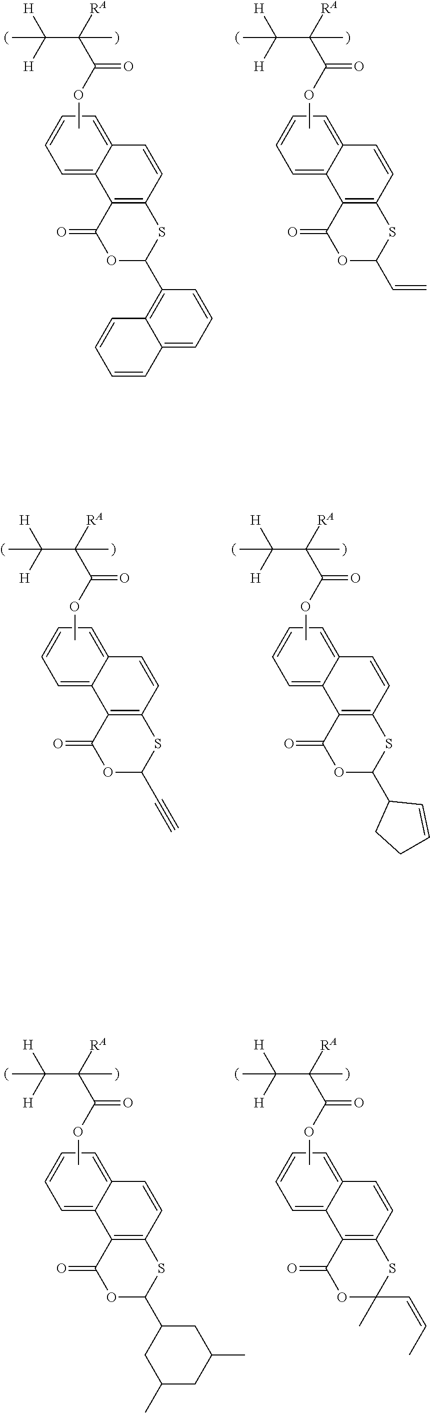

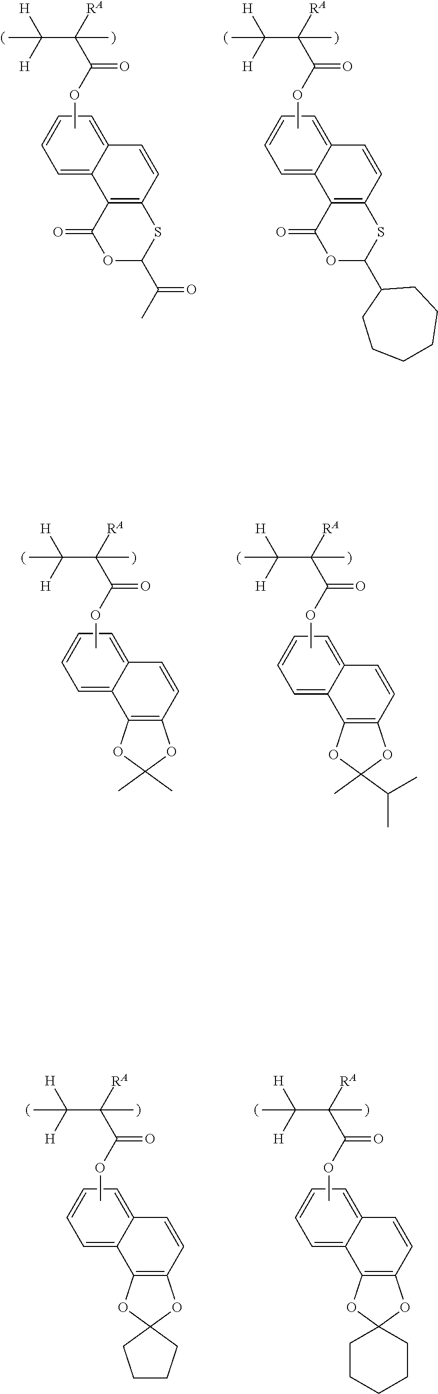

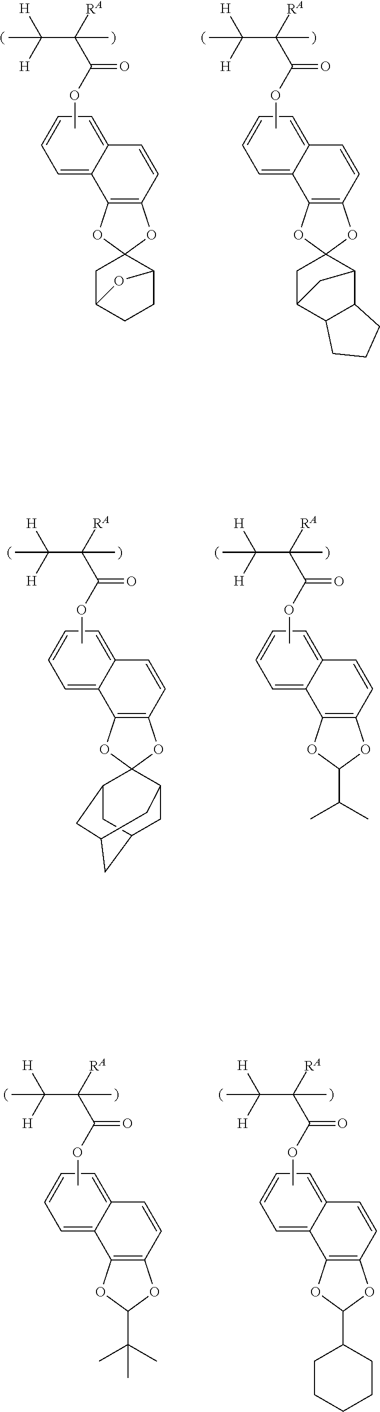

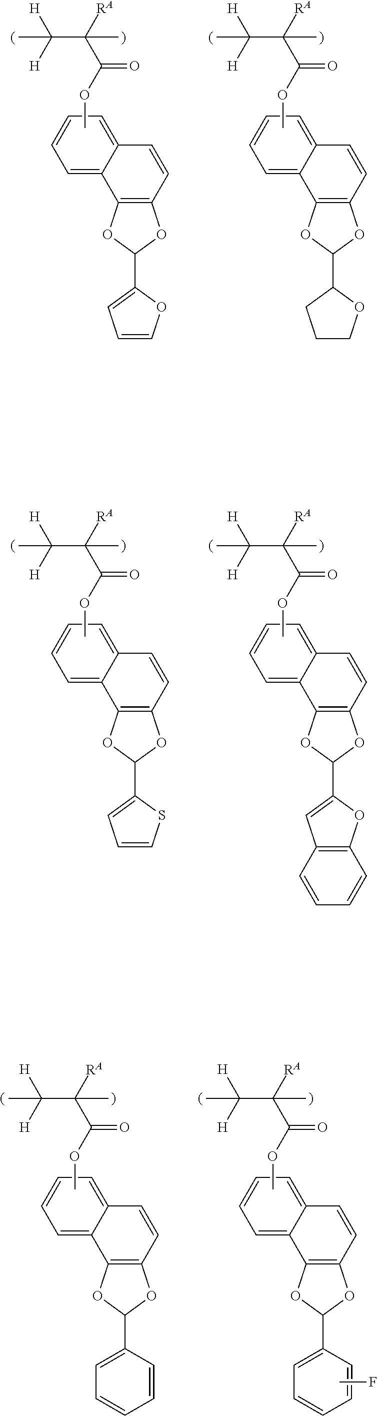

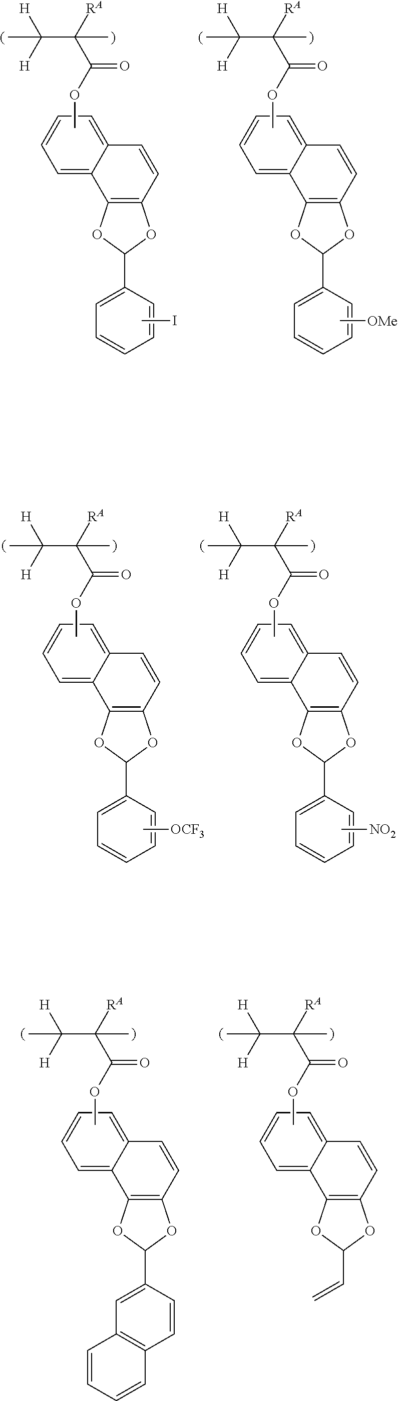

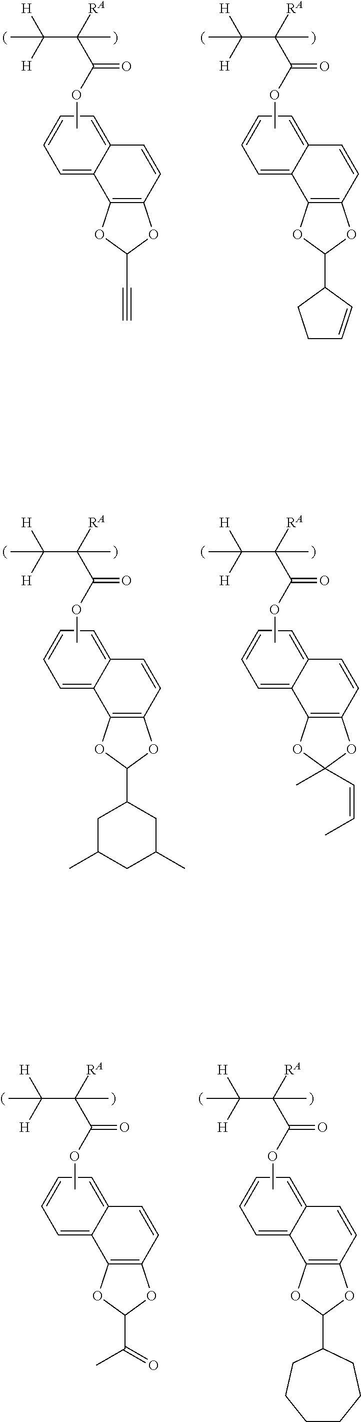

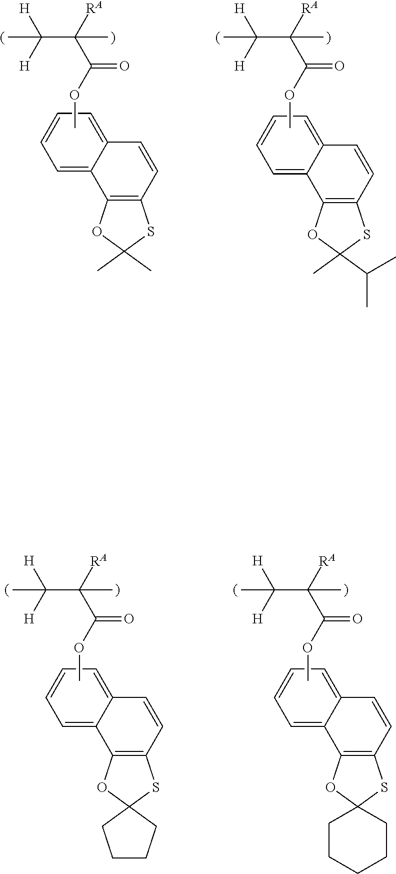

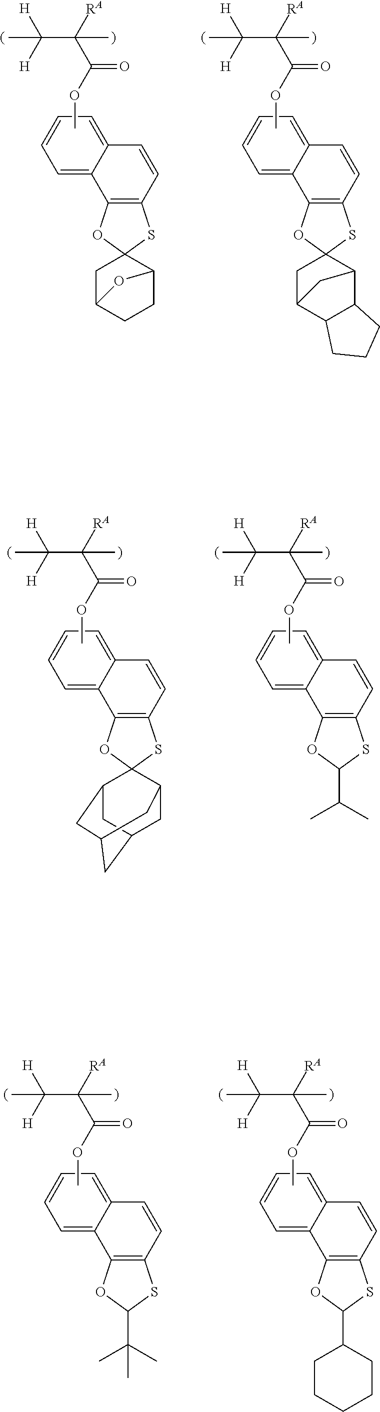

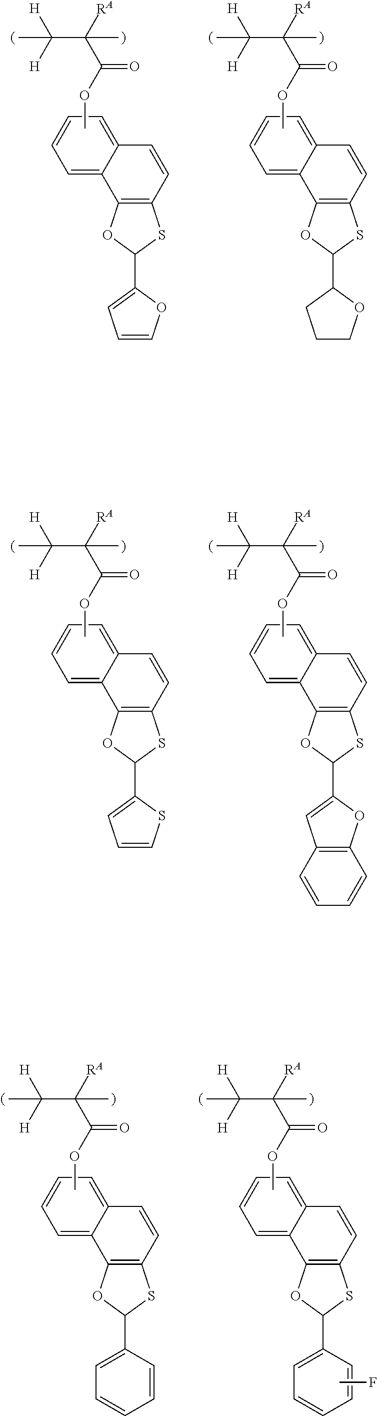

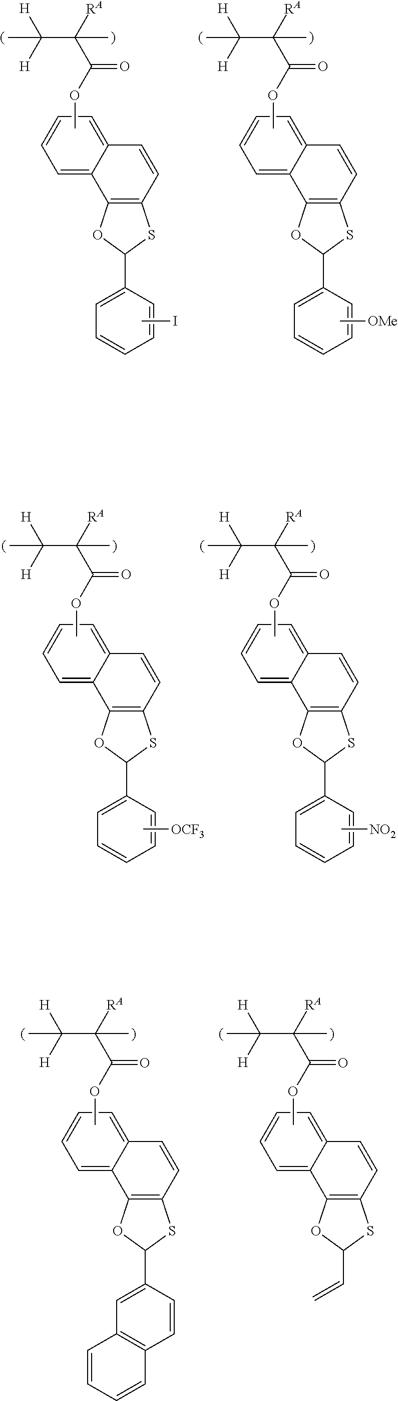

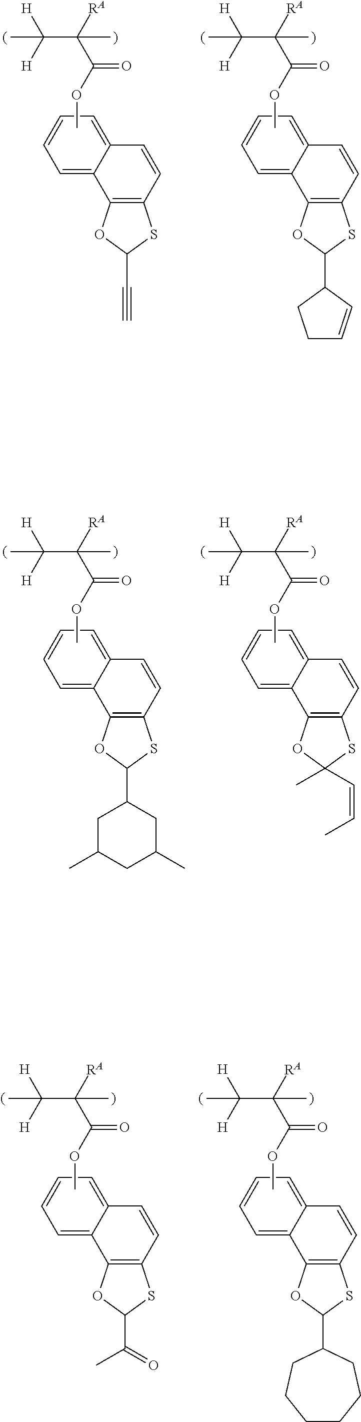

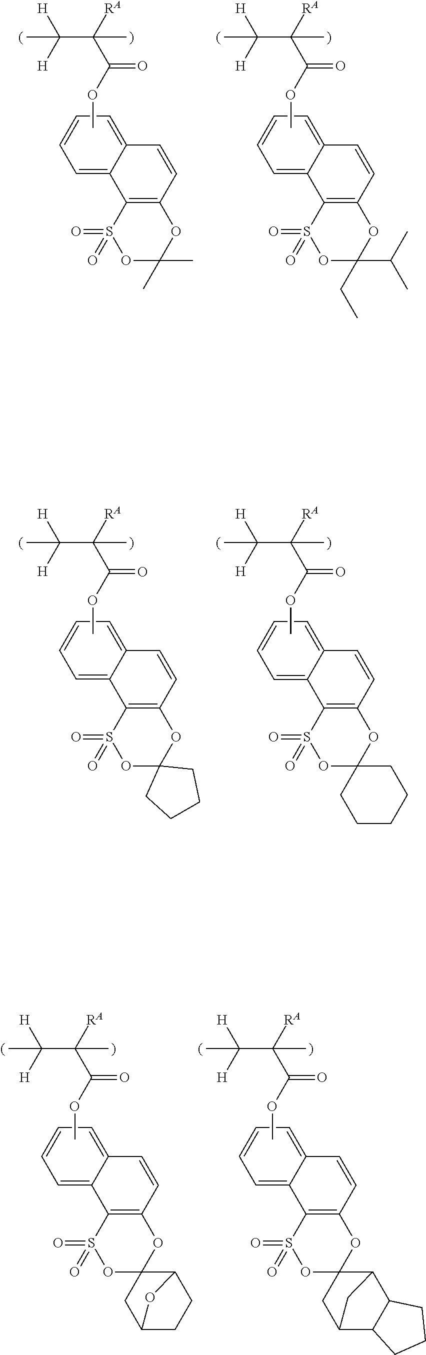

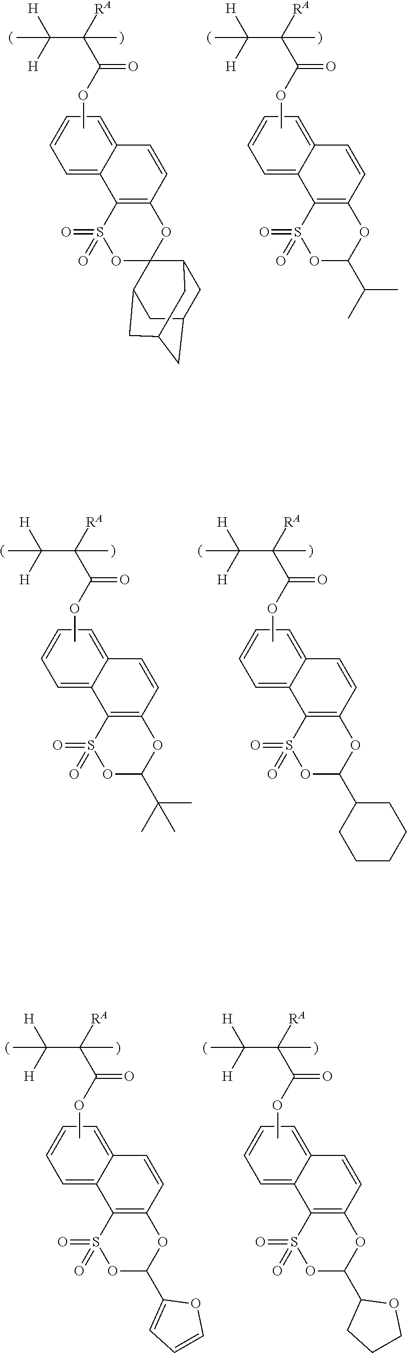

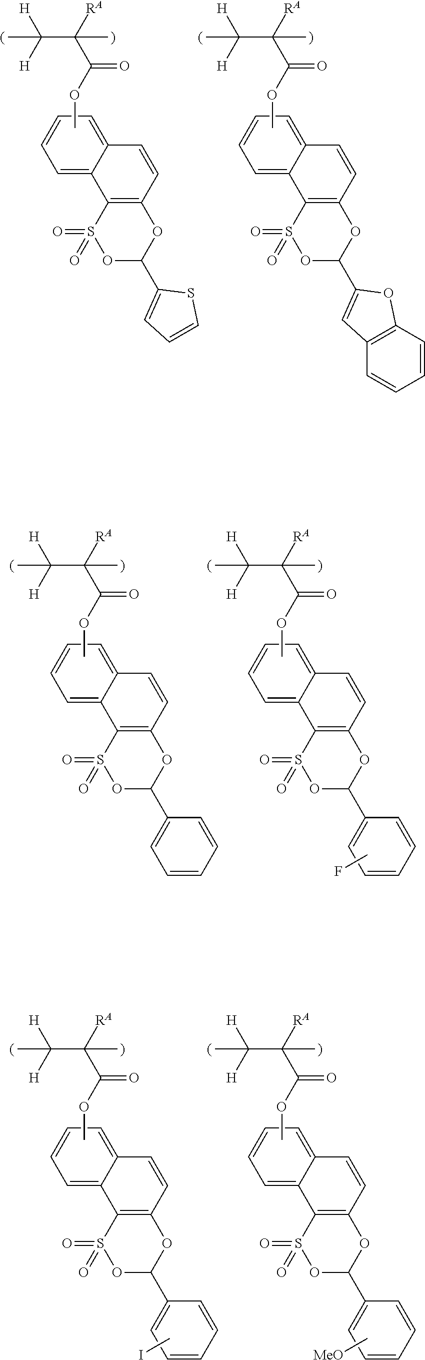

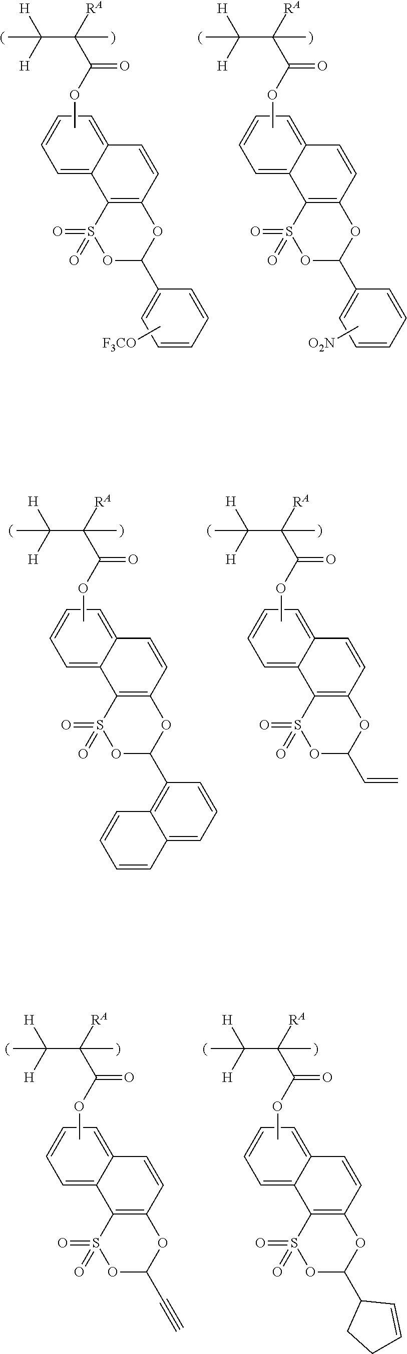

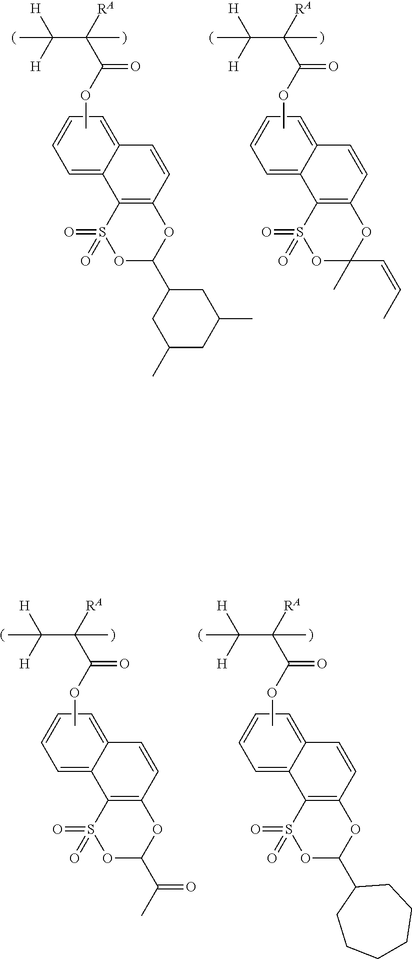

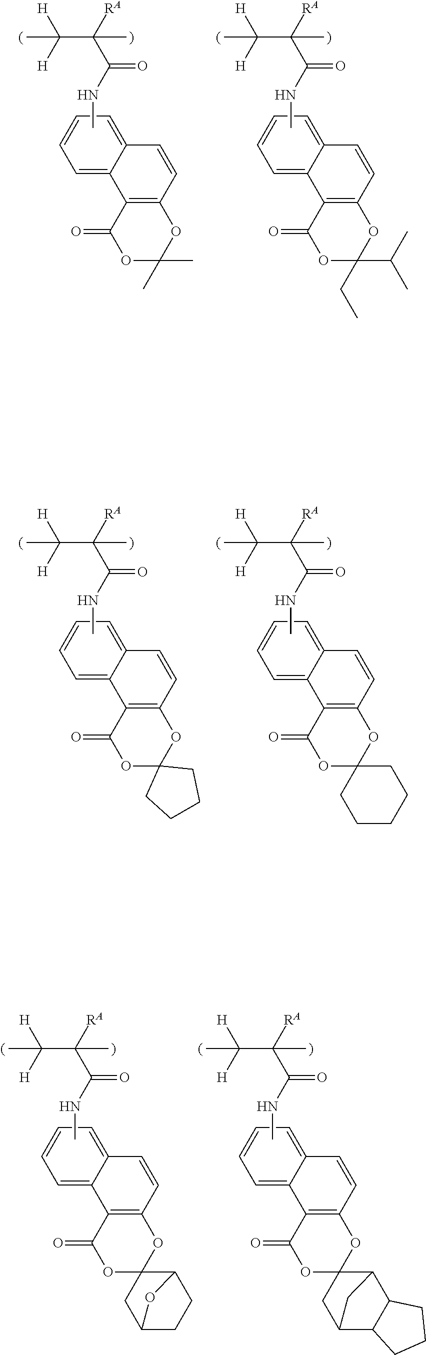

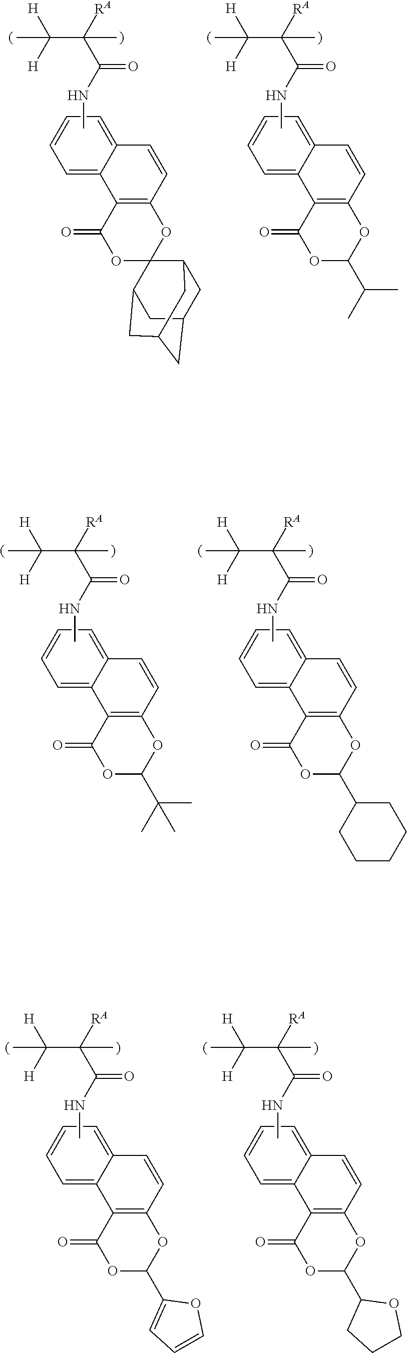

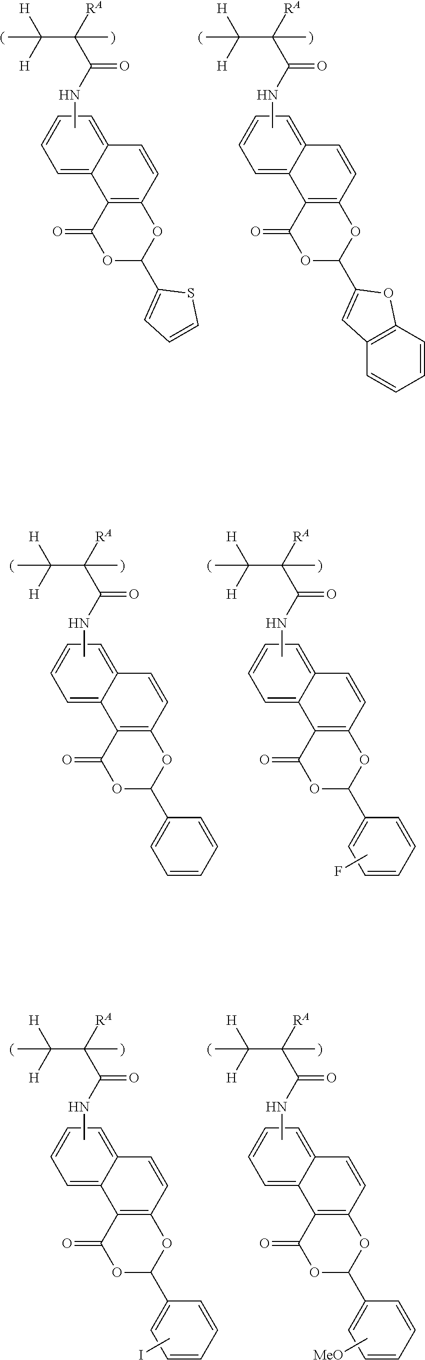

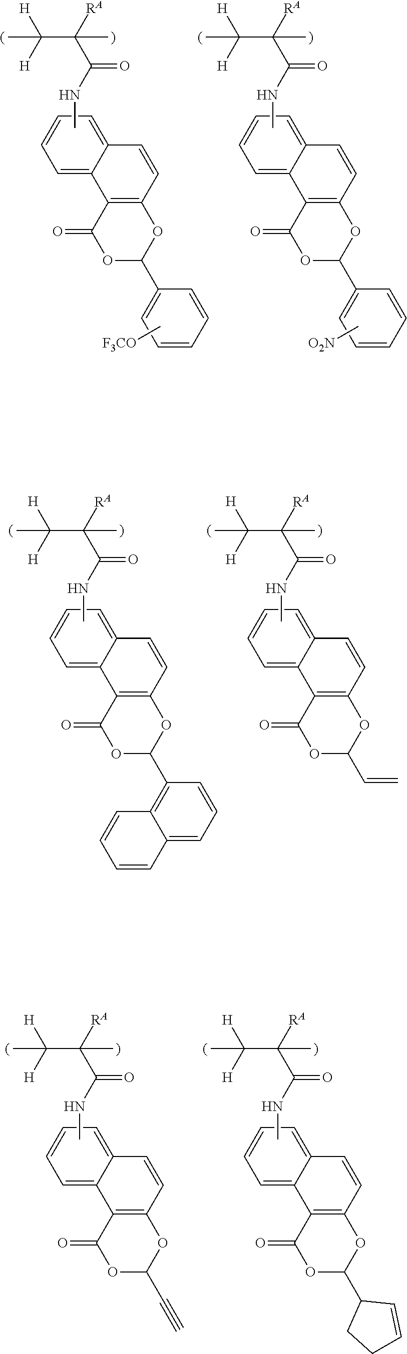

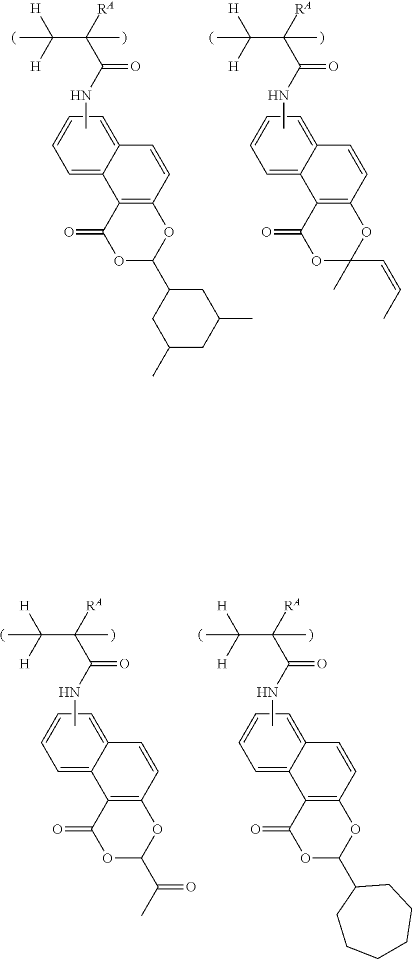

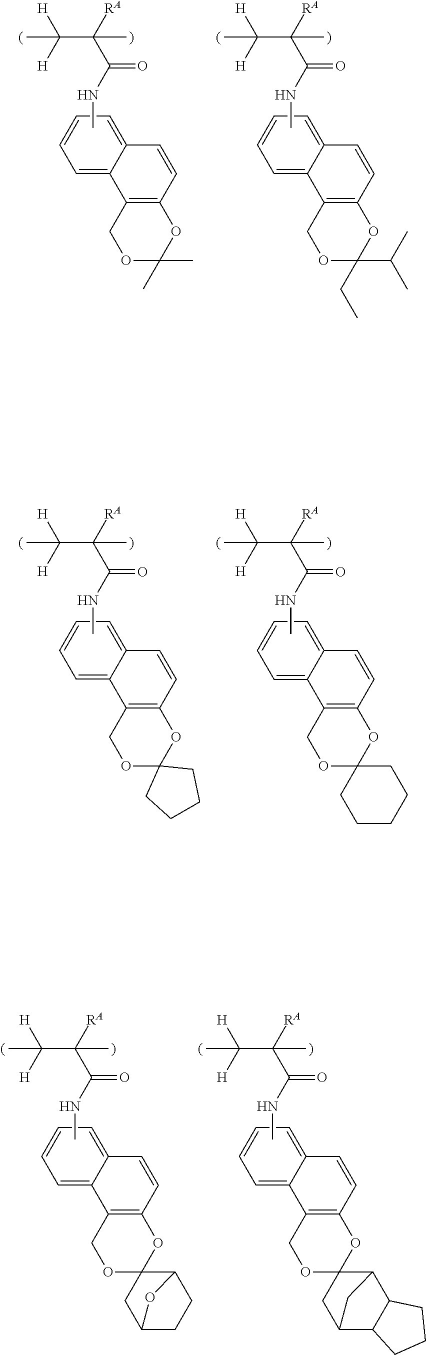

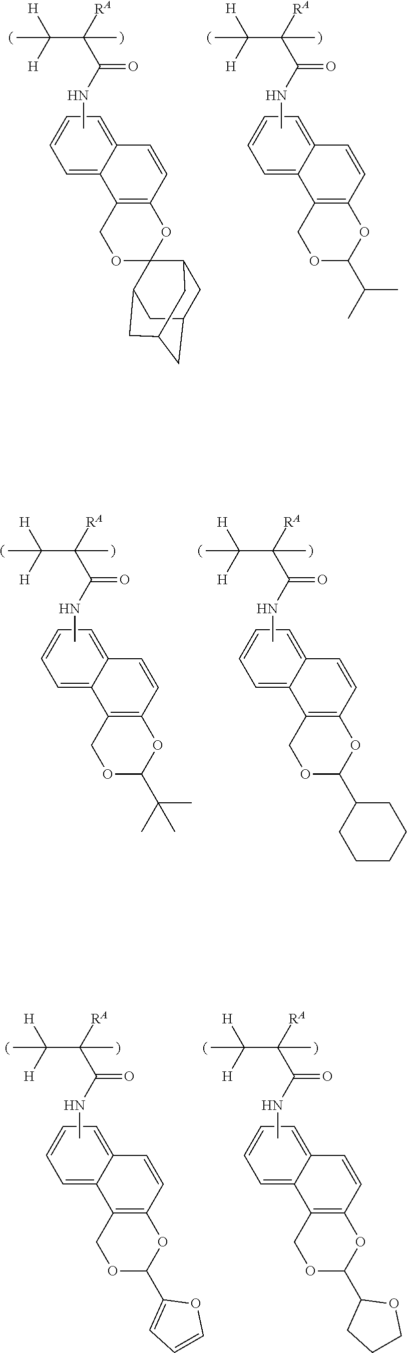

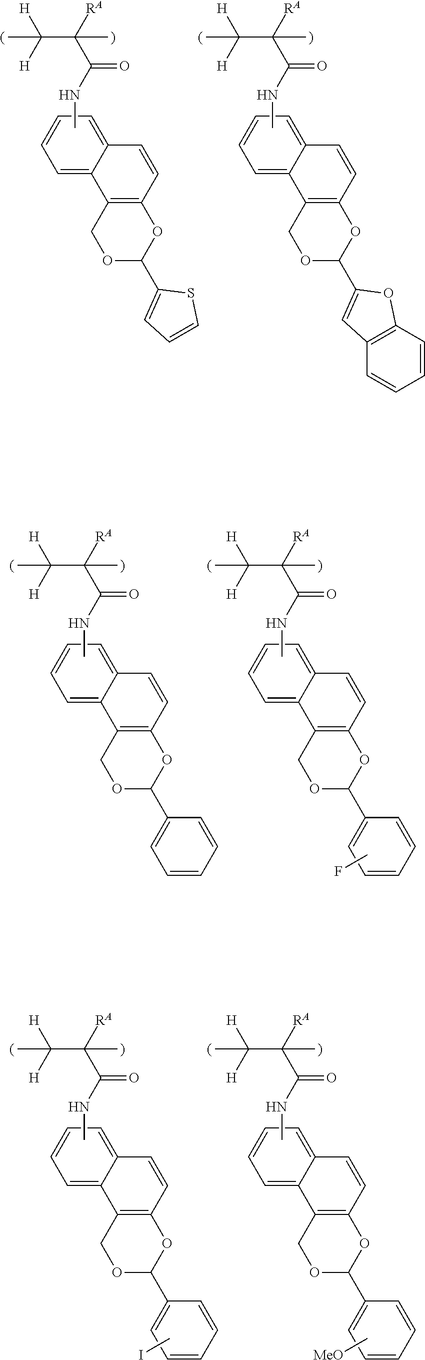

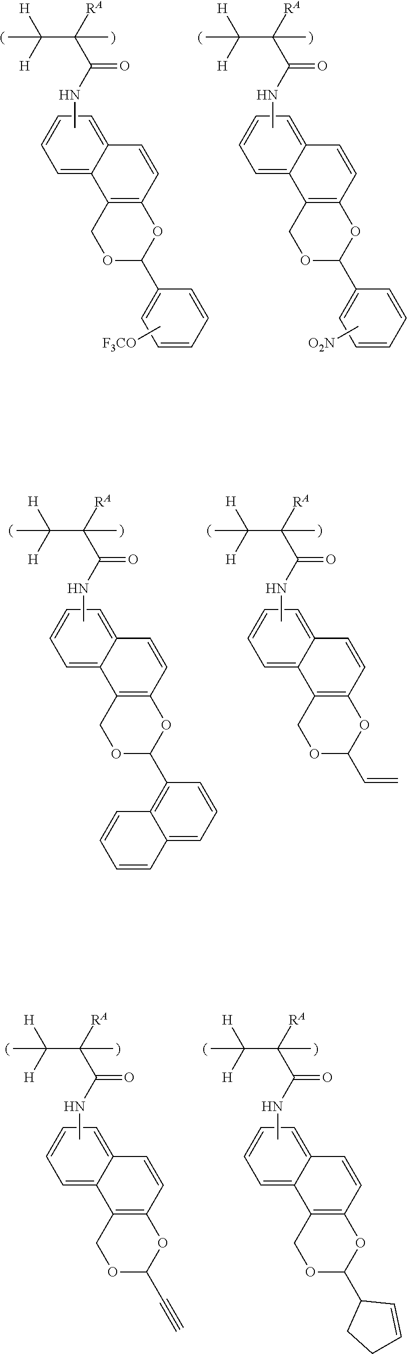

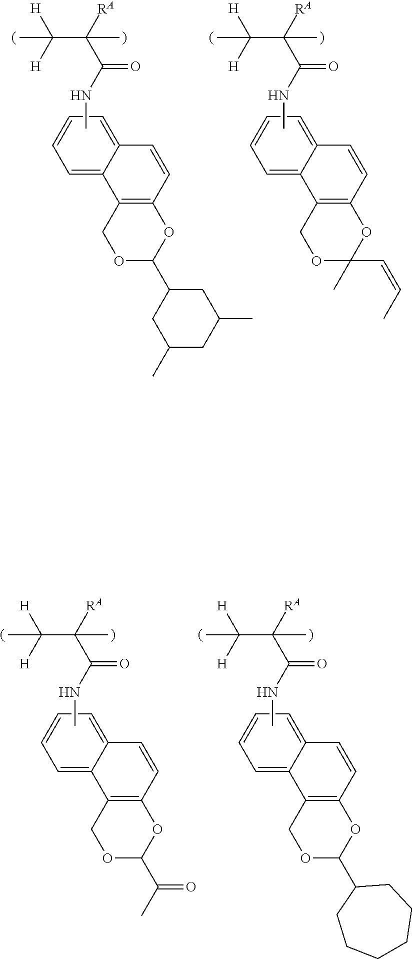

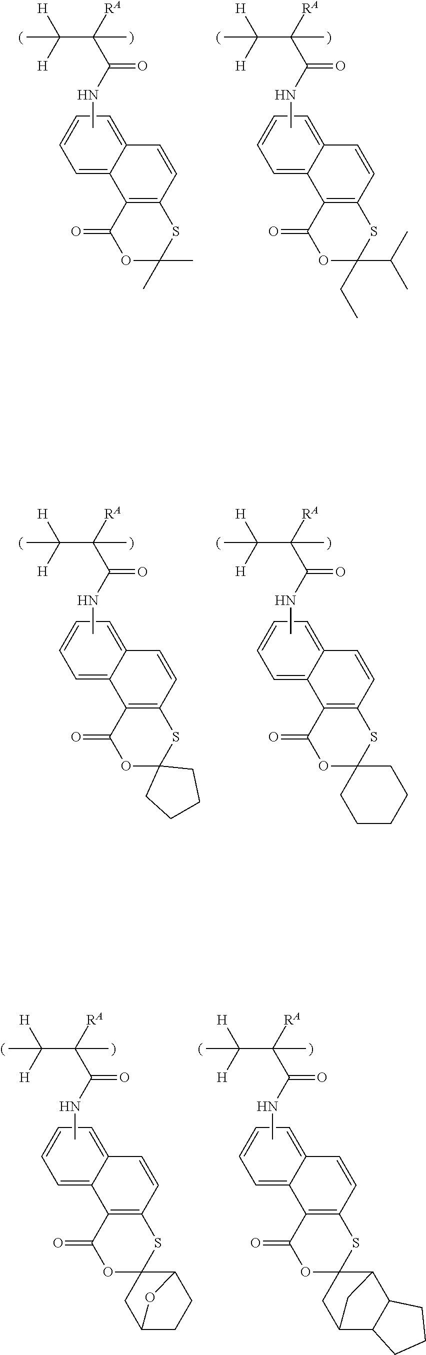

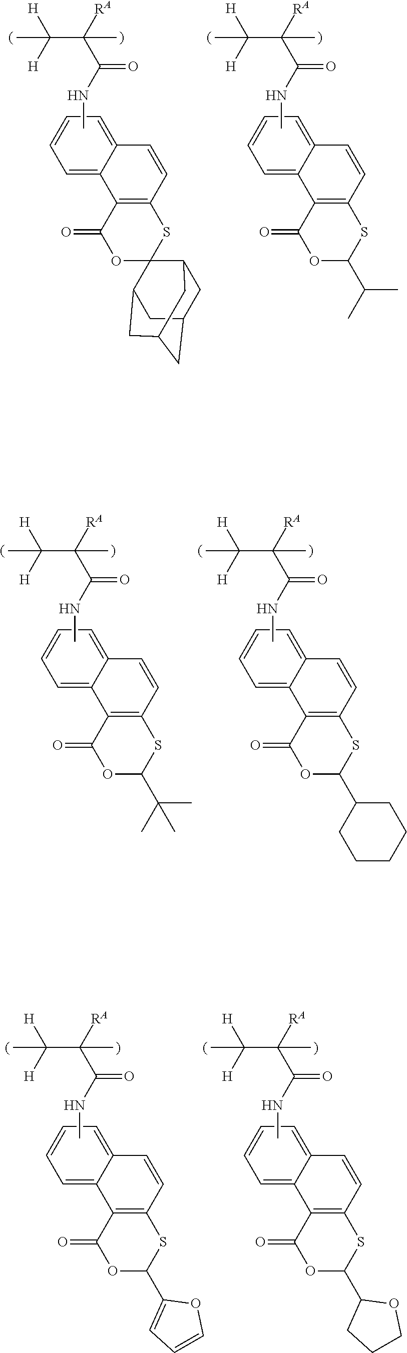

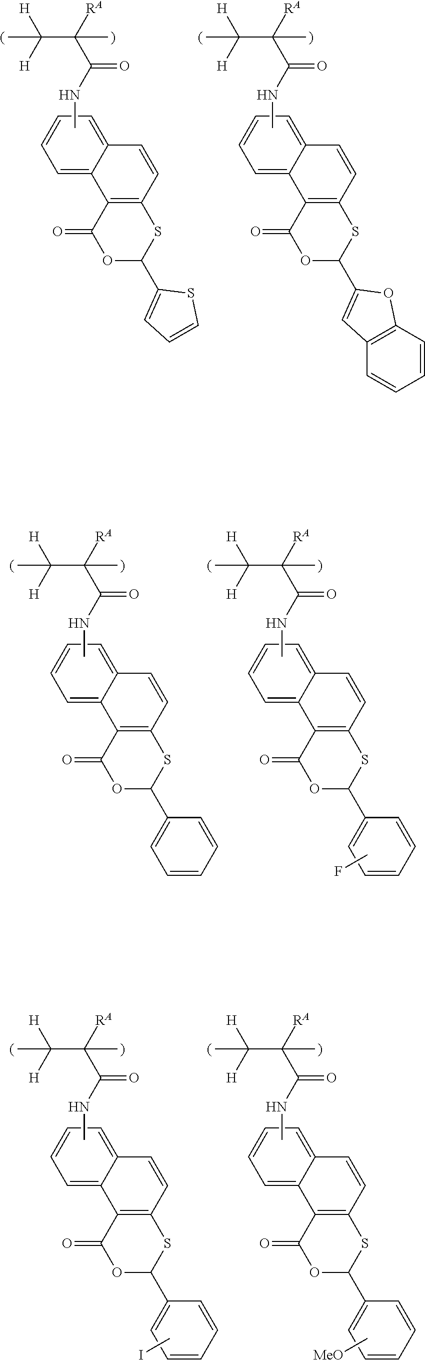

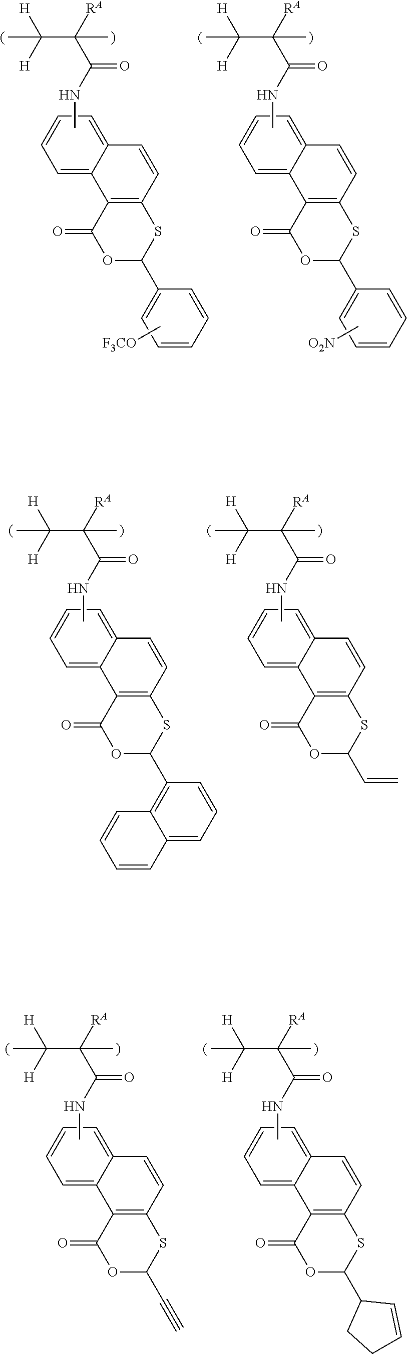

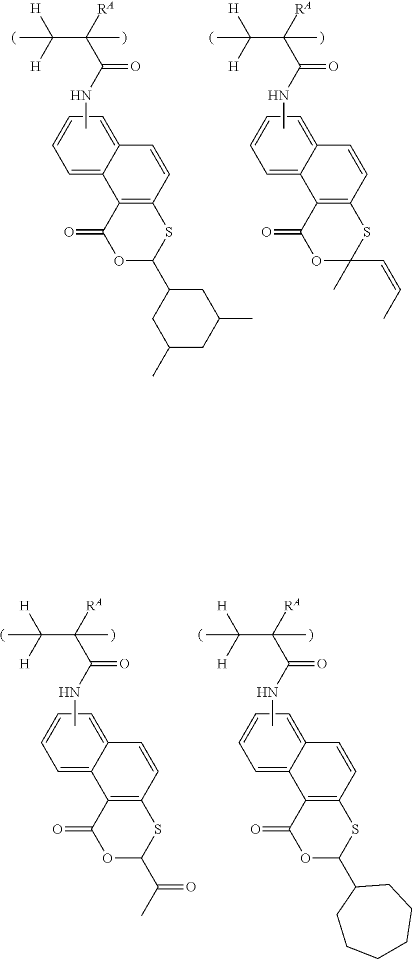

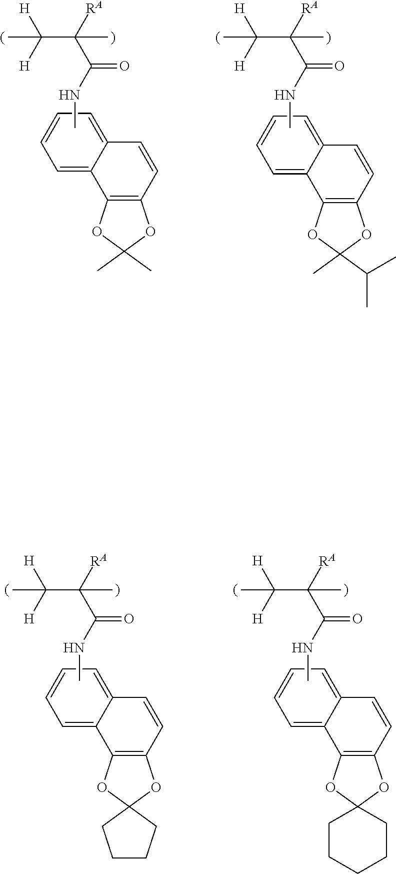

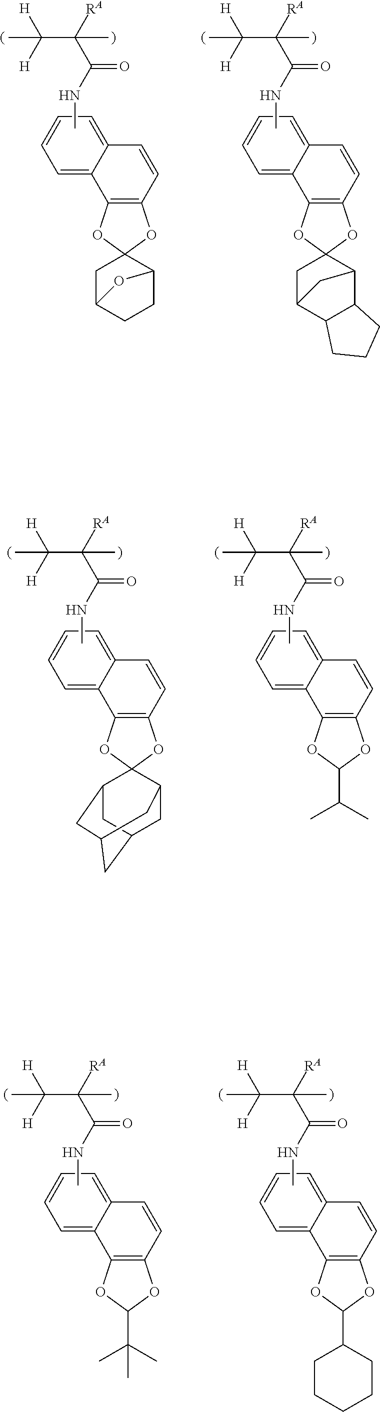

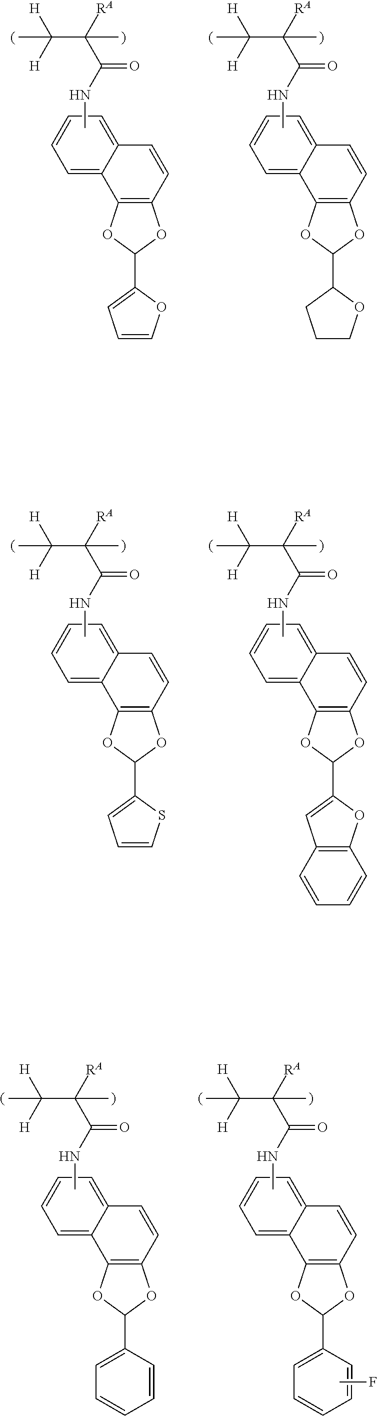

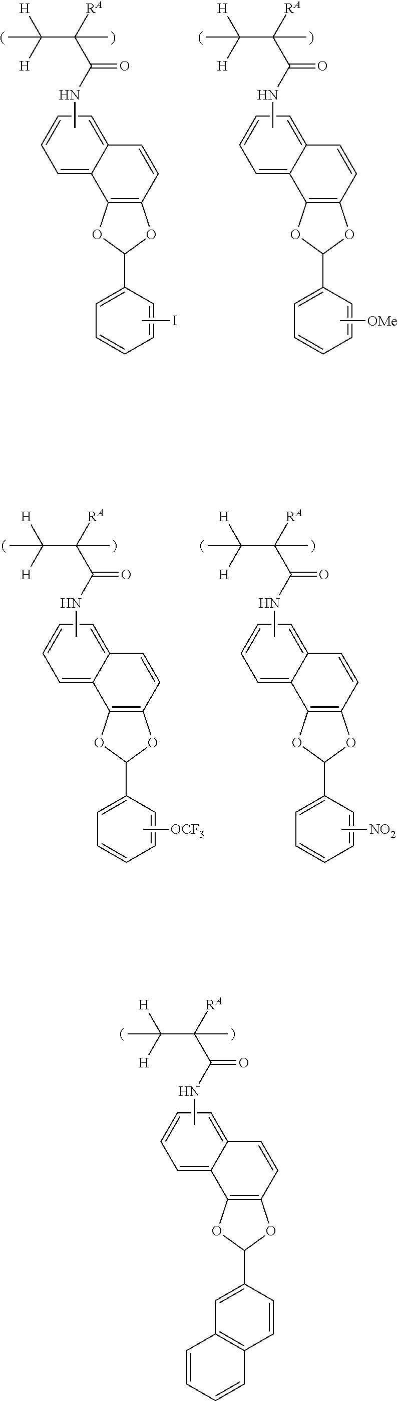

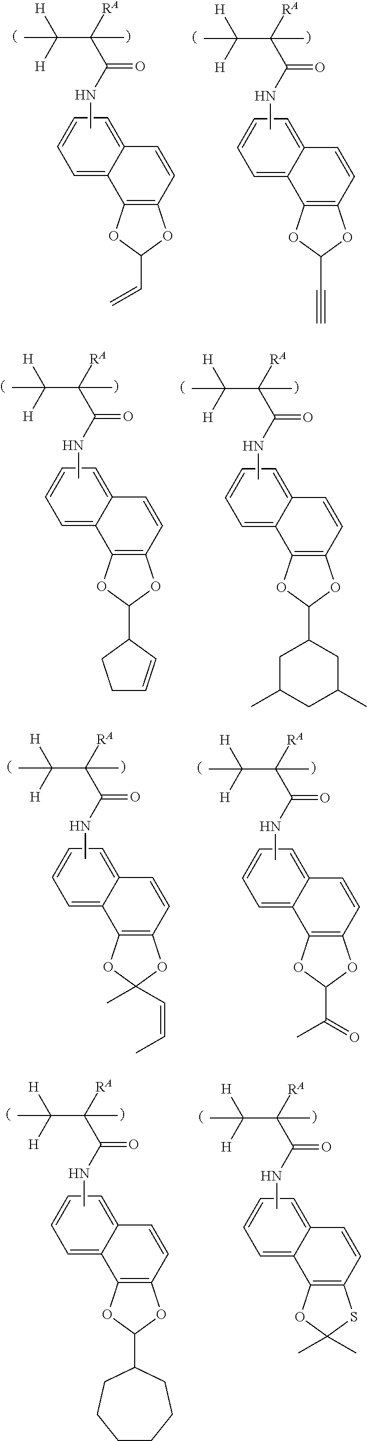

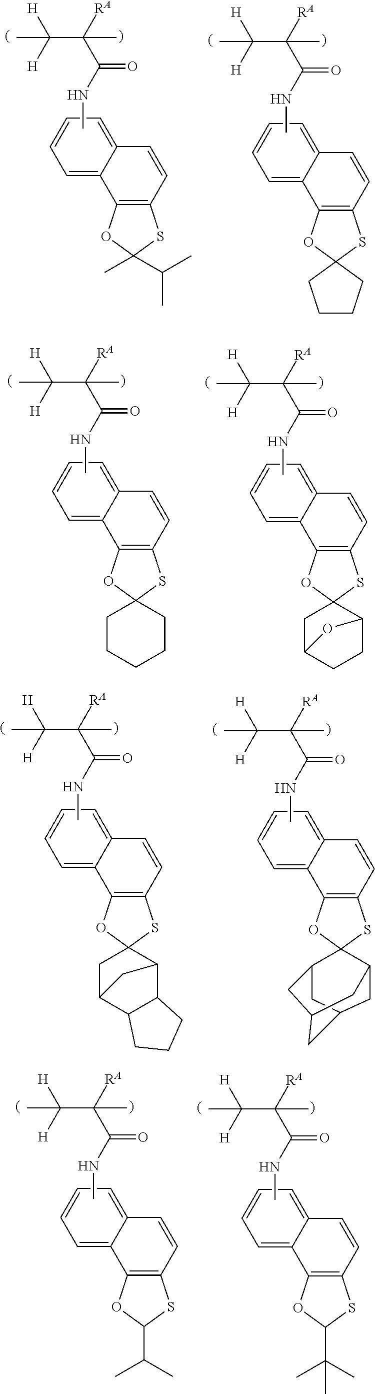

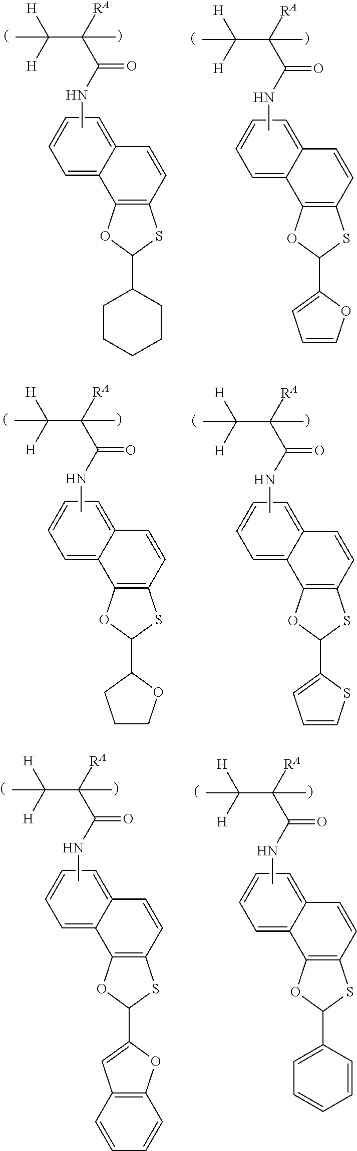

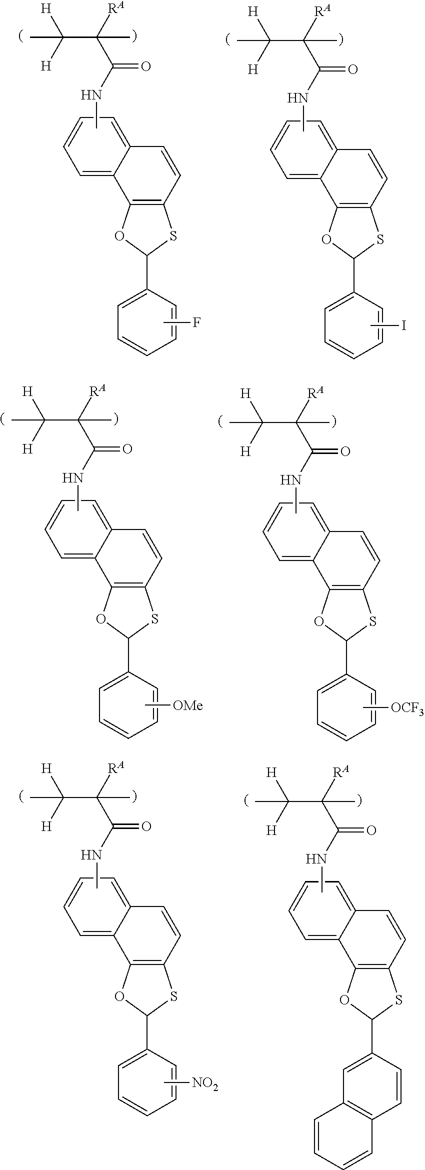

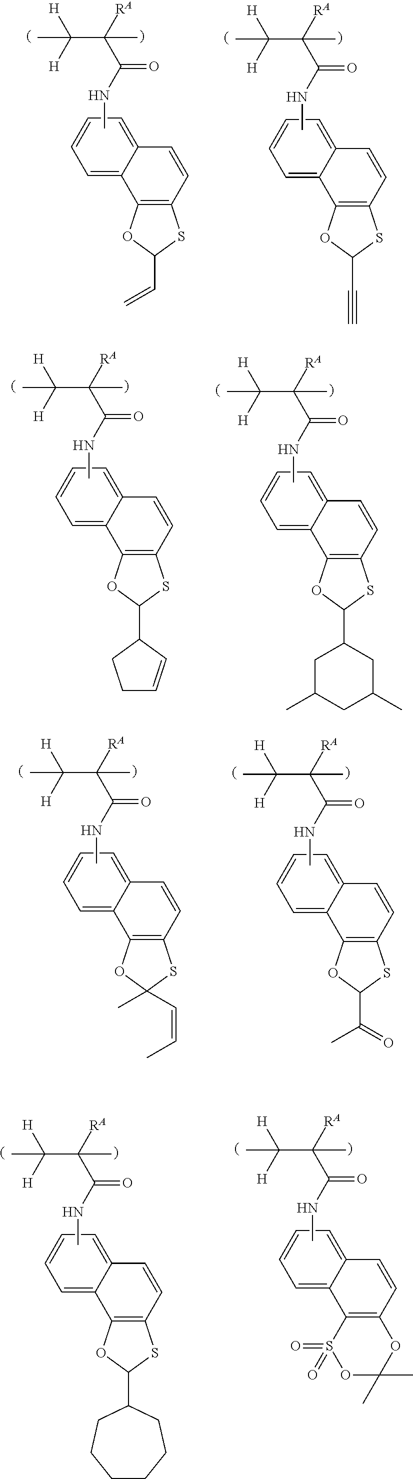

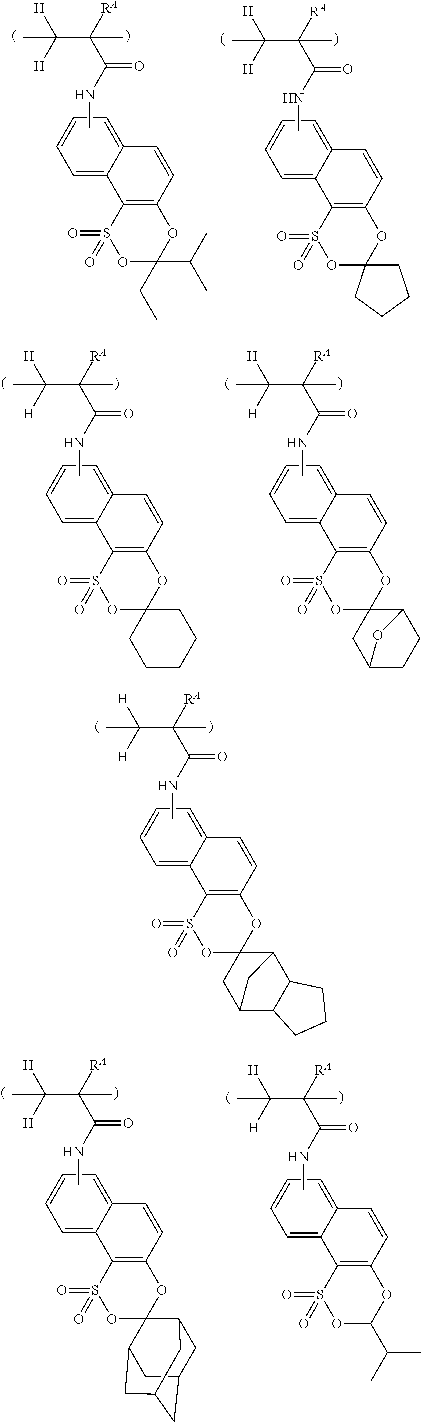

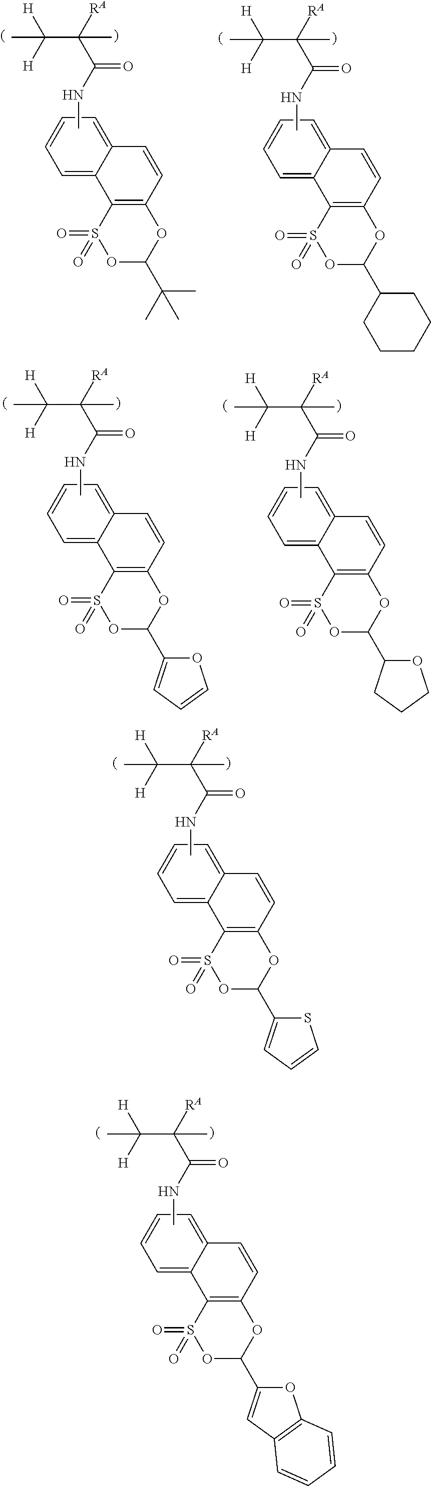

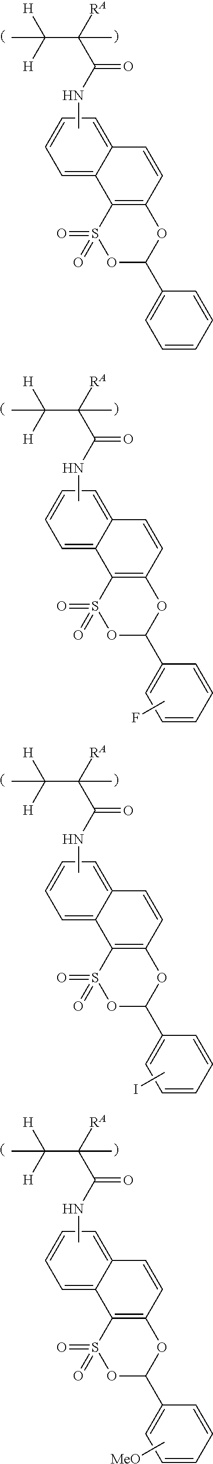

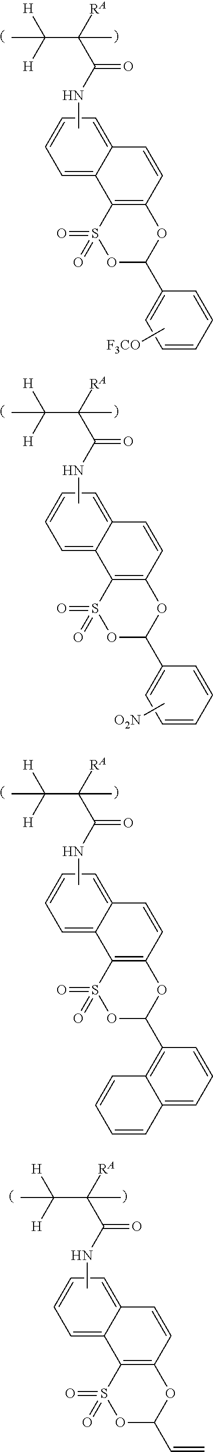

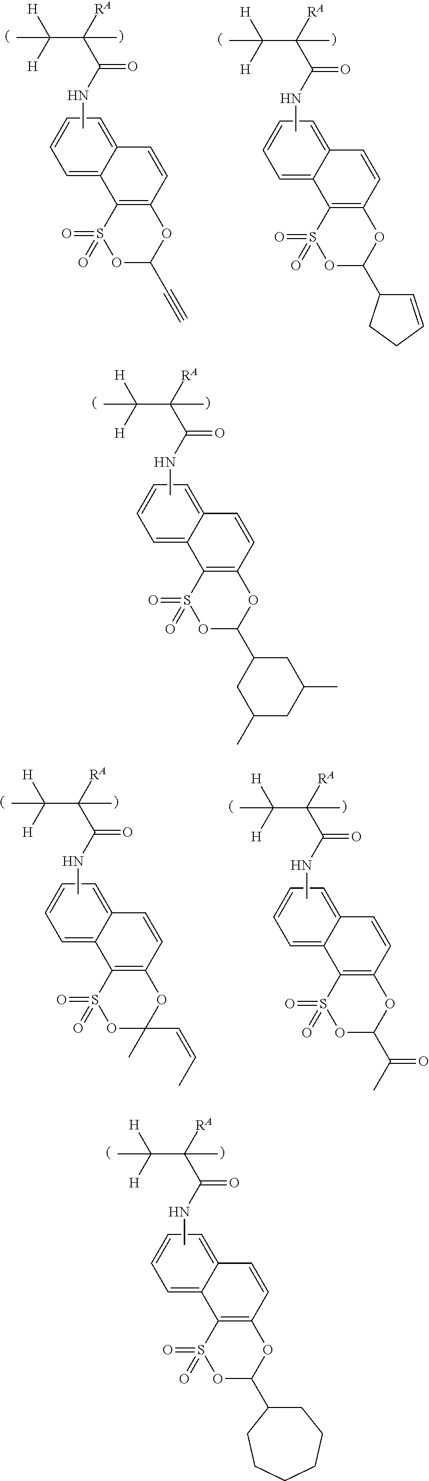

The polymer preferably has a repeating unit (P-1) having a phenolic hydroxyl group, and particularly preferably has a repeating unit represented by the following formula (5) (hereinafter also referred to as the repeating unit 5).

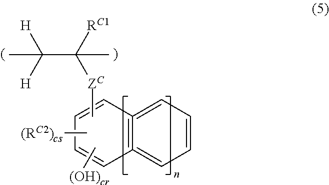

In the formula (5), R^{C1} represents a hydrogen atom or a methyl group. Z^{C} represents a single bond or an ester bond. R^{C2} represents a fluorine atom, an iodine atom, a trifluoromethyl group, a trifluoromethoxy group, or a monovalent organic group having 1 to 10 carbon atoms and optionally having a heteroatom, and “—CH₂—” in the organic group may be substituted with “—O—” or “—C(═O)—”. cs represents an integer of 0 to 4. cr represents an integer of 1 to 5. “n” represents 0 or 1.

Specific examples of the repeating unit 5 include those shown below, but are not limited thereto. In the following formulae, R^{C1} is the same as defined above.

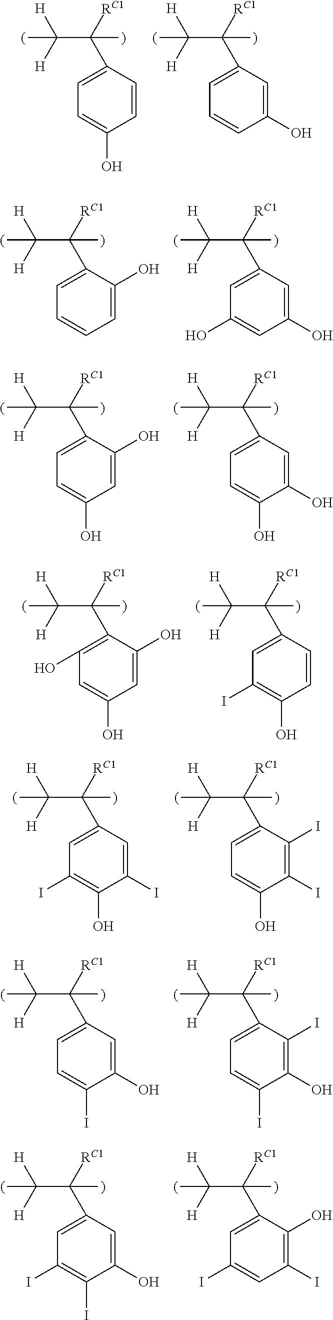

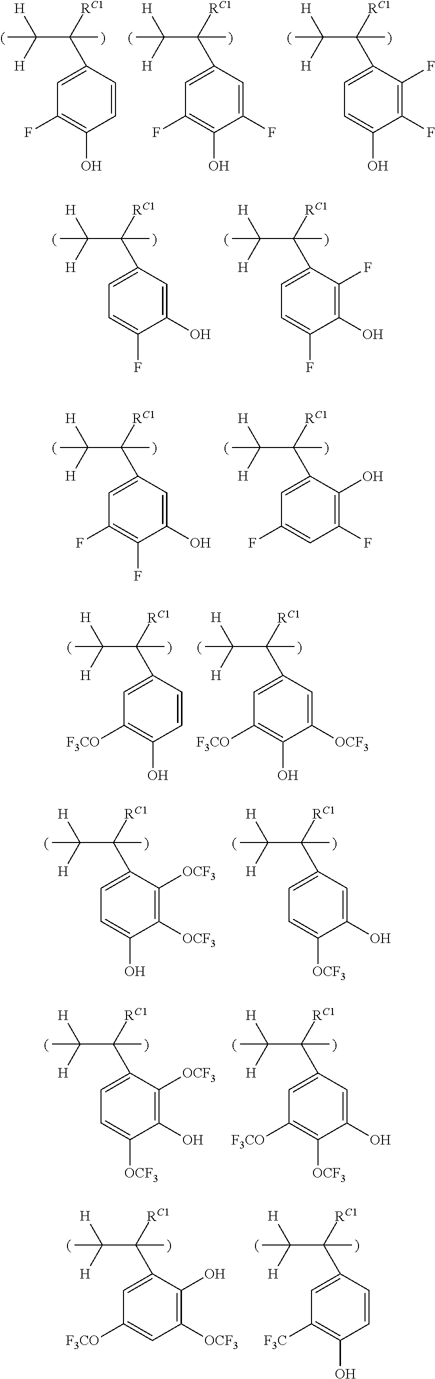

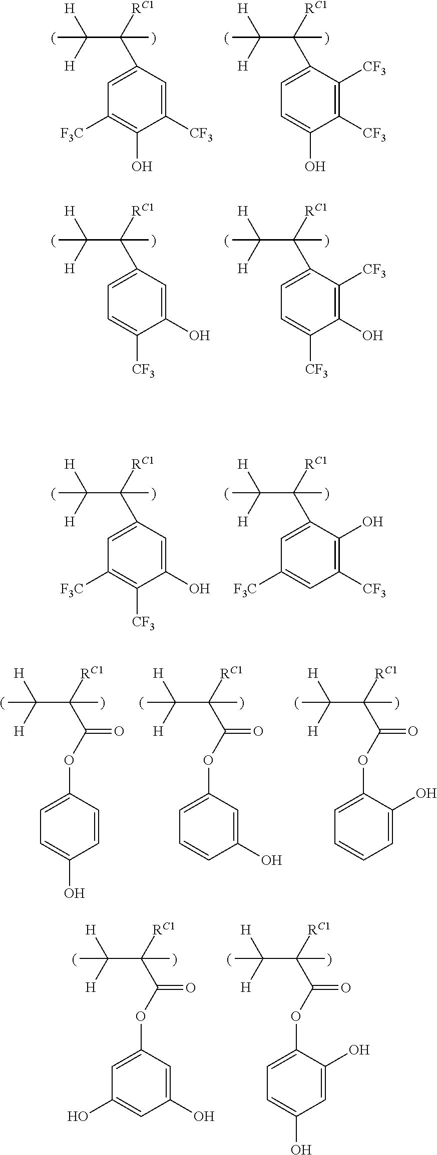

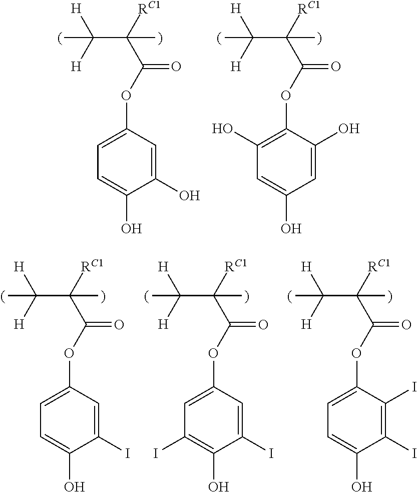

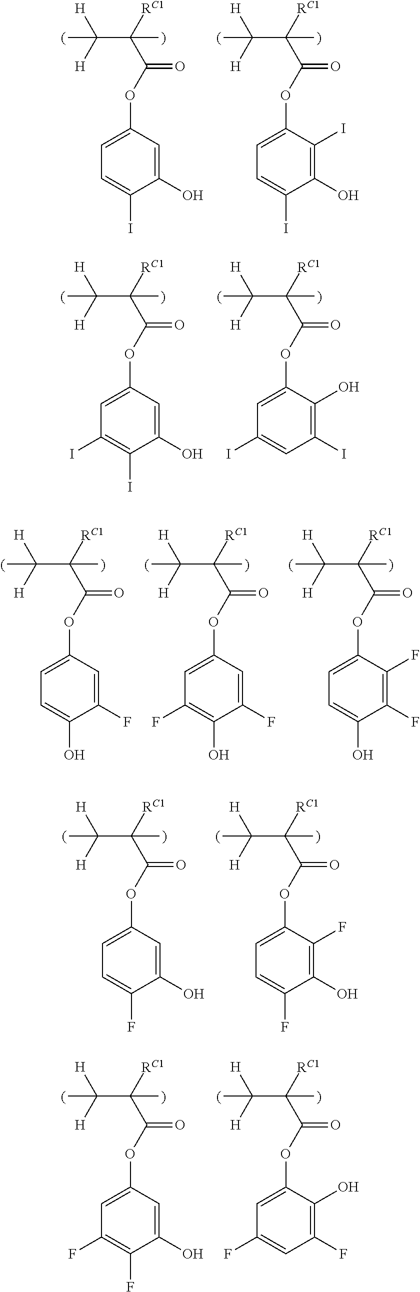

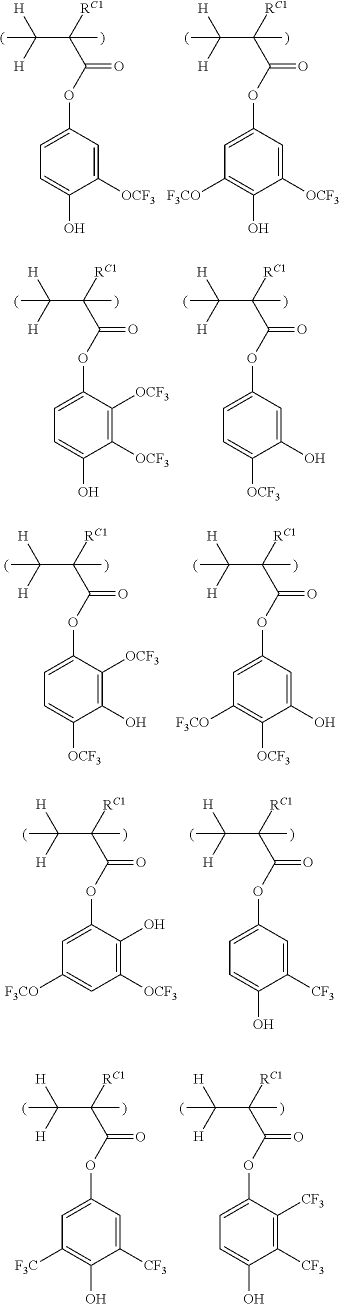

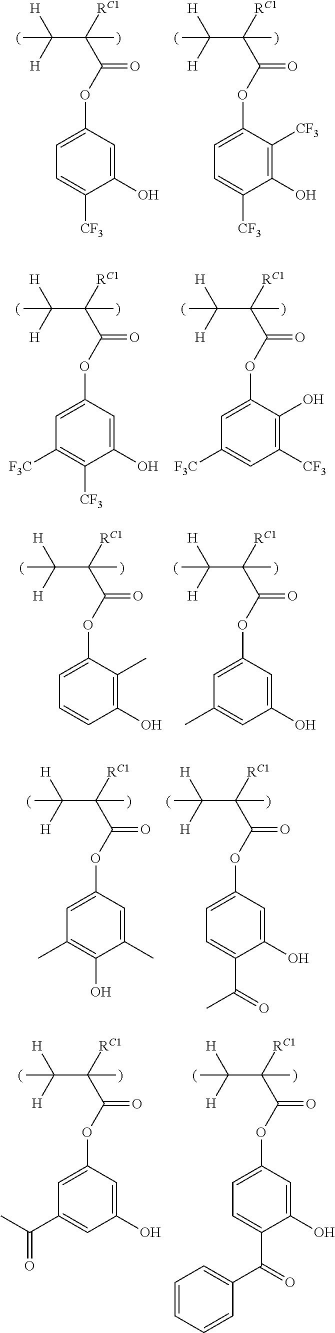

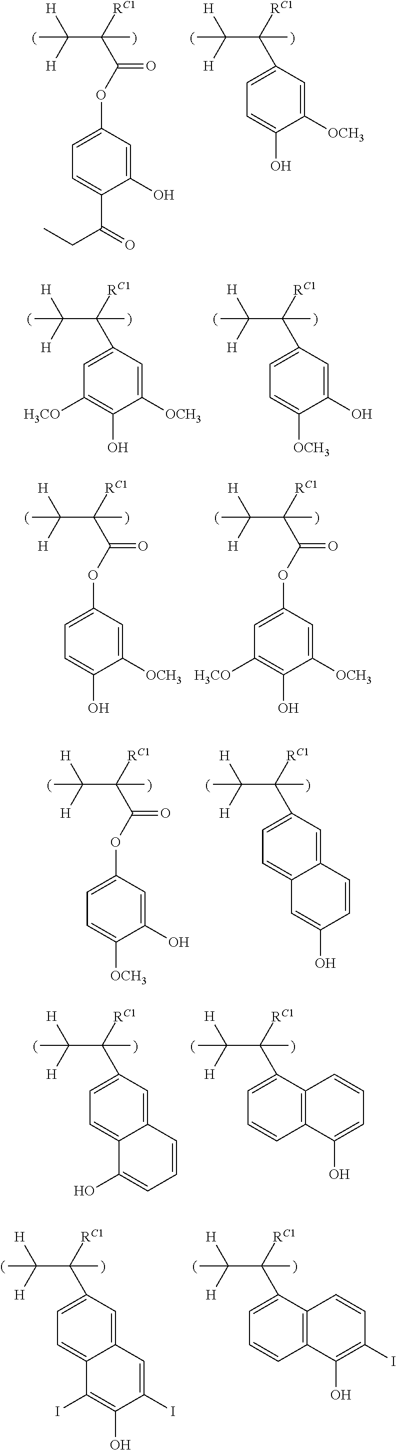

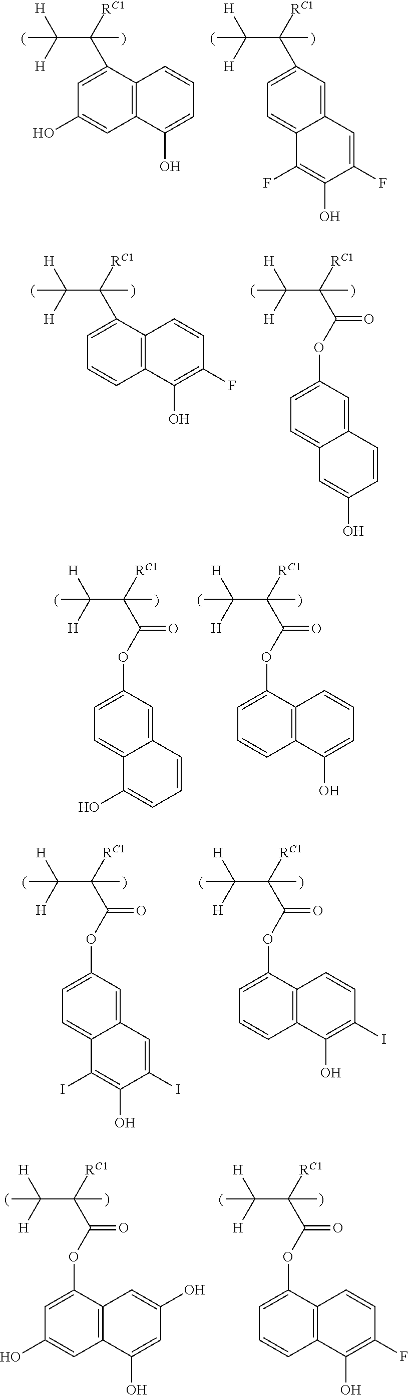

The polymer may further have a repeating unit represented by the following formula (d1) (hereinafter also referred to as the repeating unit “d”).

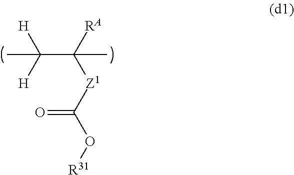

In the formula (d1), R^{A} is a hydrogen atom, a fluorine atom, a methyl group, or a trifluoromethyl group. Z¹ is a single bond, a phenylene group, a naphthylene group, “*—C(═O)—O—Z¹¹—” or “*—C(═O)—NH—Z¹¹—”, and the phenylene group or the naphthylene group may be substituted with a hydroxy group, a nitro group, a cyano group, a saturated hydrocarbyl group having 1 to 10 carbon atoms and optionally having a fluorine atom, an alkoxy group having 1 to 10 carbon atoms optionally having a fluorine atom, or a halogen atom. “*” represents a bond to a carbon atom of the main chain. Z¹¹ represents a saturated hydrocarbylene group having 1 to 10 carbon atoms, a phenylene group, or a naphthylene group, and the saturated hydrocarbylene group may have a hydroxy group, an ether bond, an ester bond, or a lactone ring. R³¹ represents a hydrogen atom, or a group having 1 to 20 carbon atoms having at least one structure selected from the group consisting of a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—).

Specific examples of the repeating unit “d” include those shown below, but are not limited thereto. In the following formulae, R^{A} is the same as defined above.

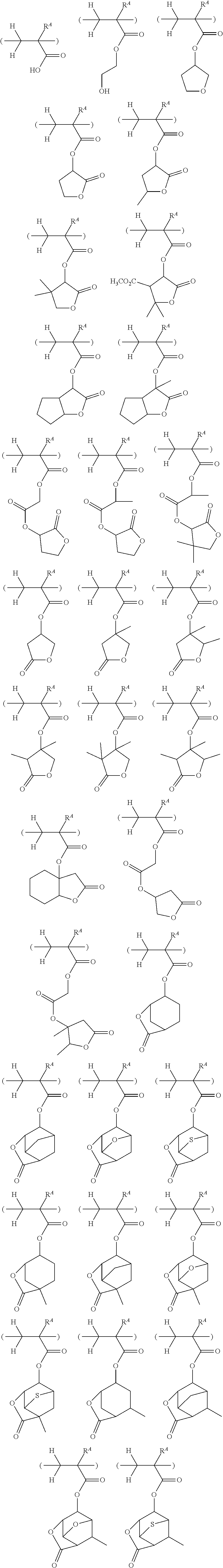

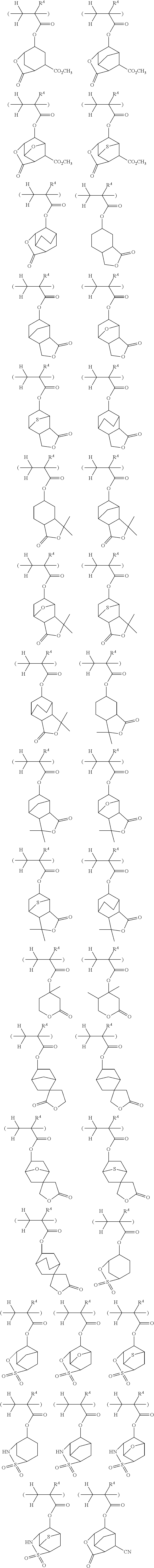

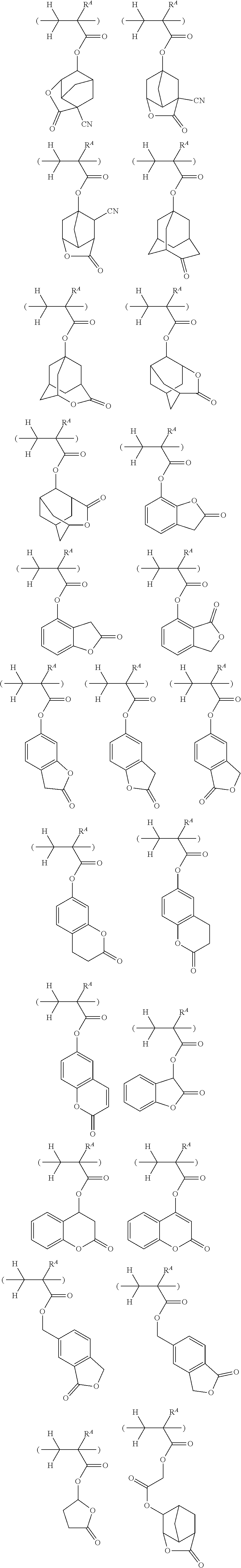

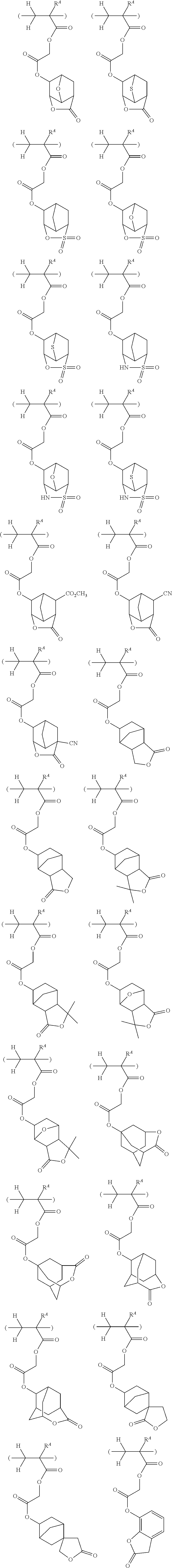

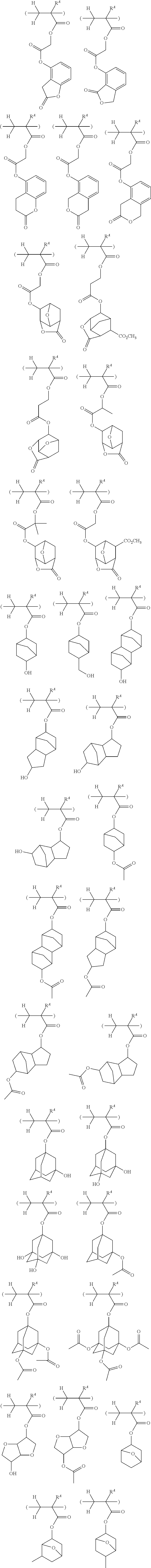

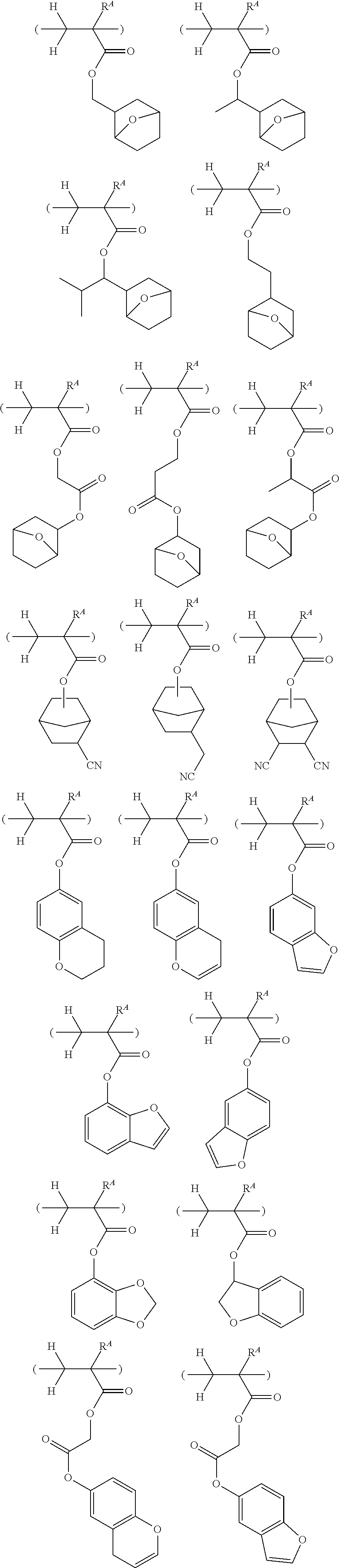

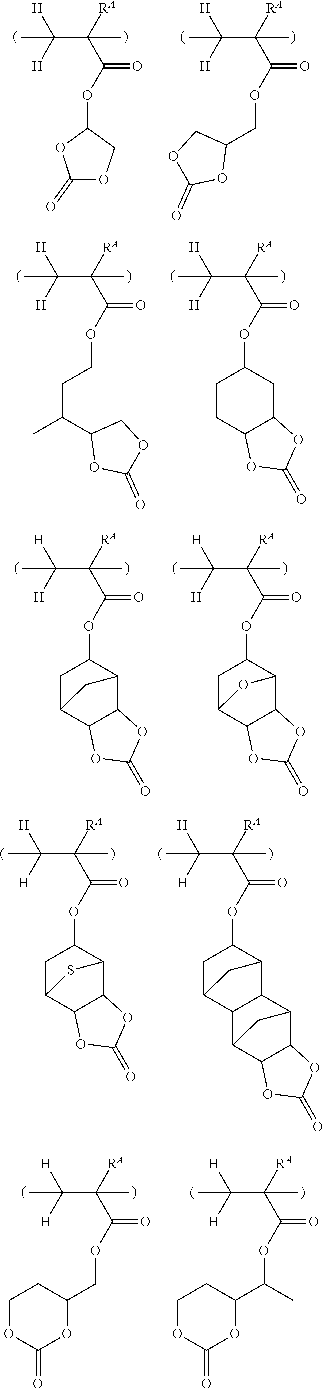

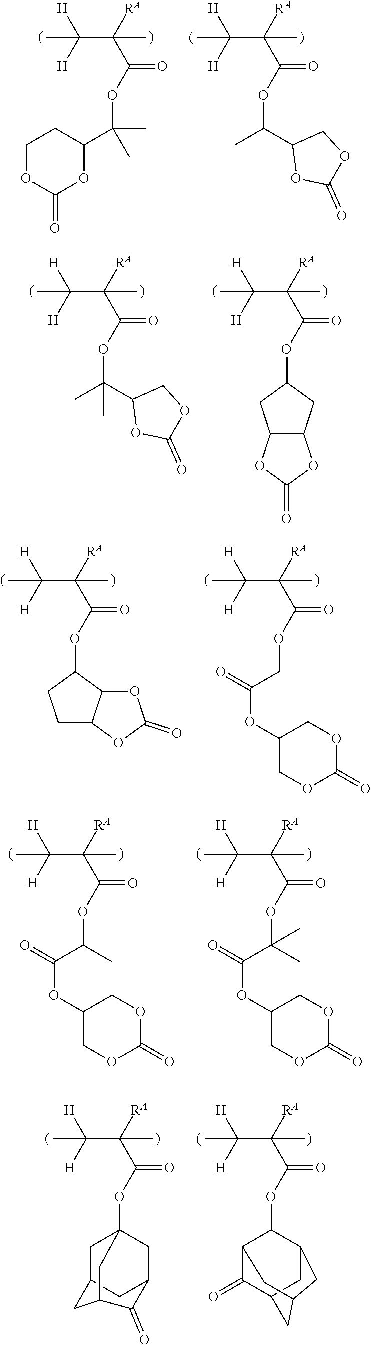

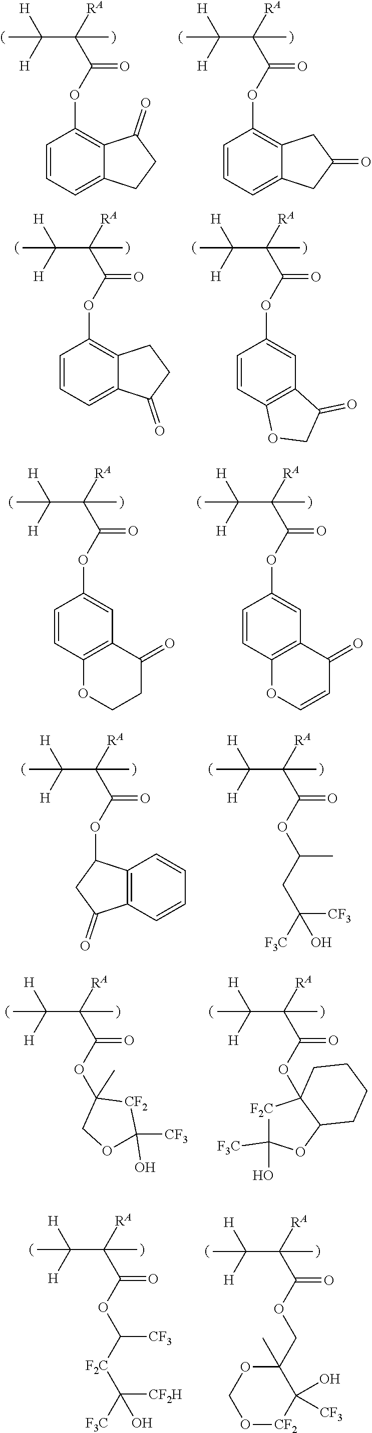

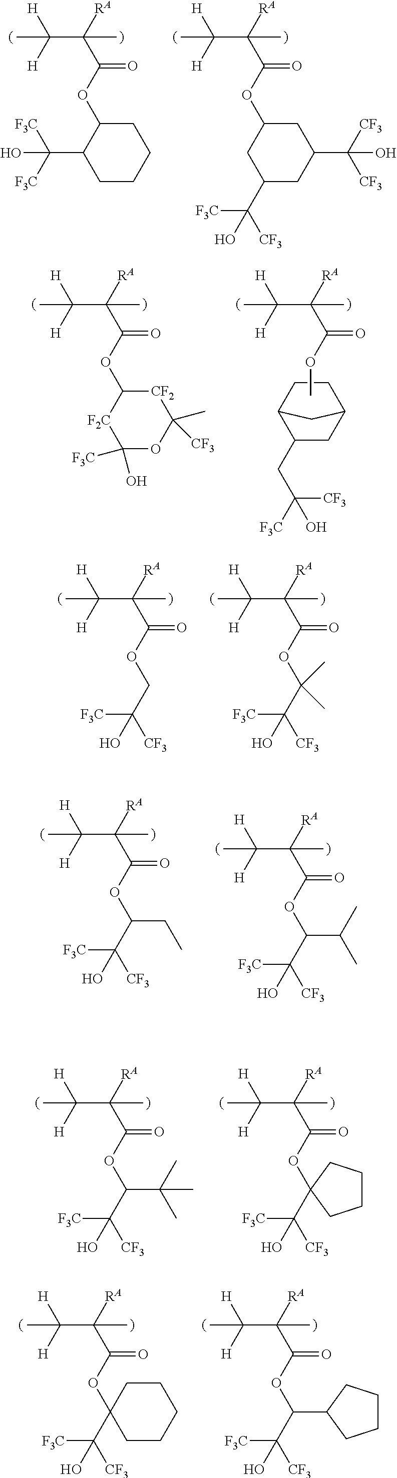

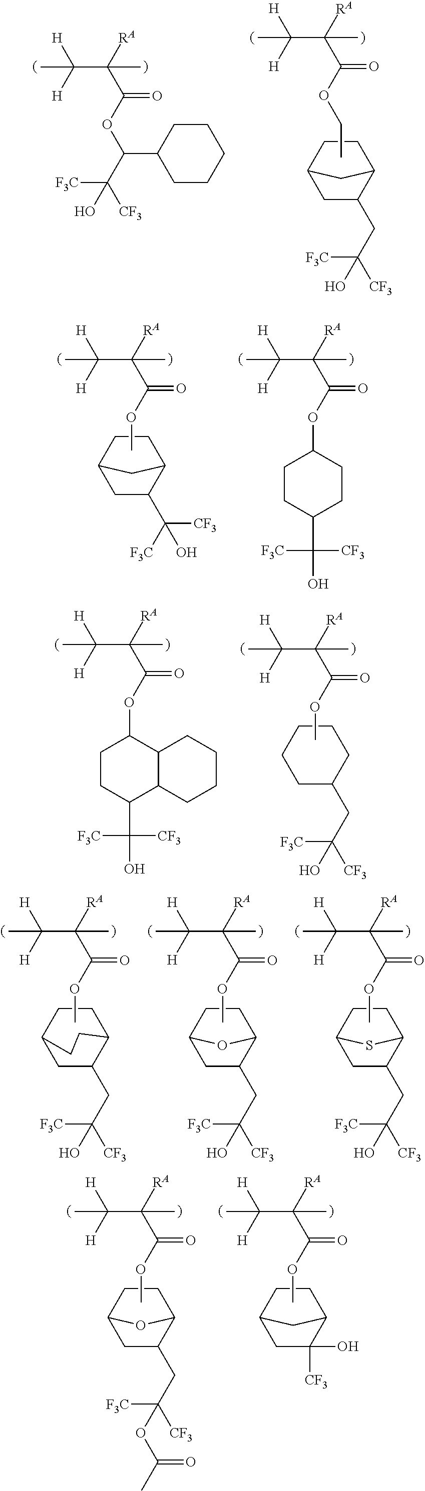

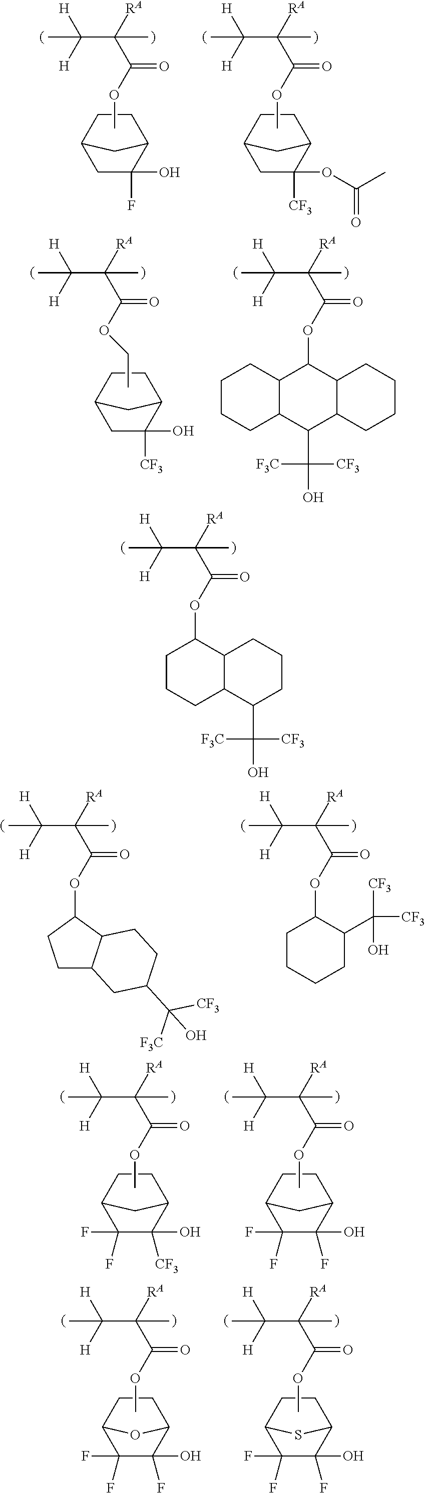

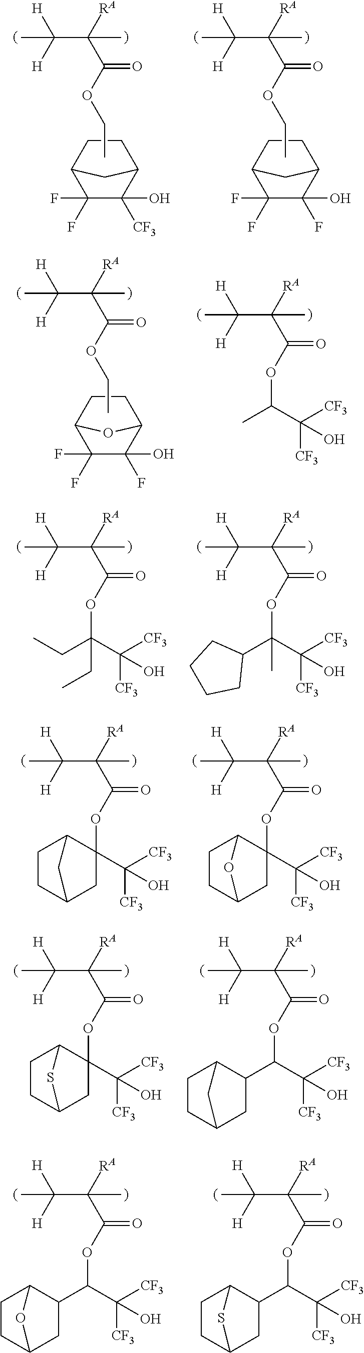

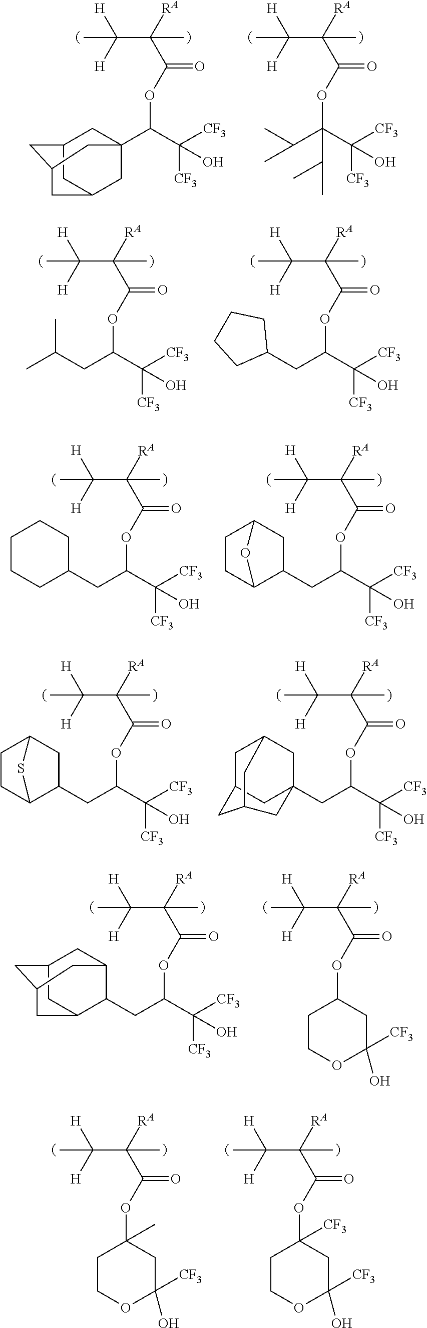

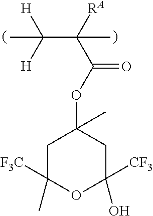

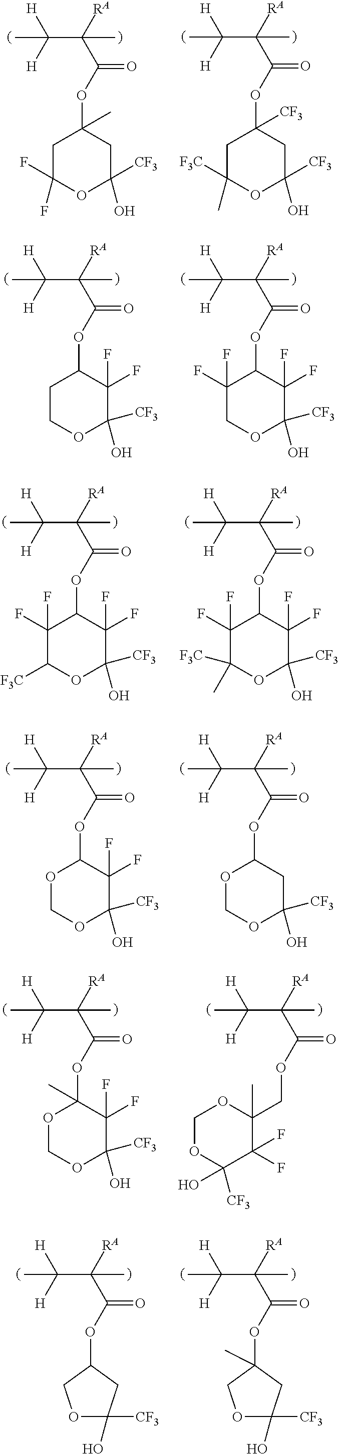

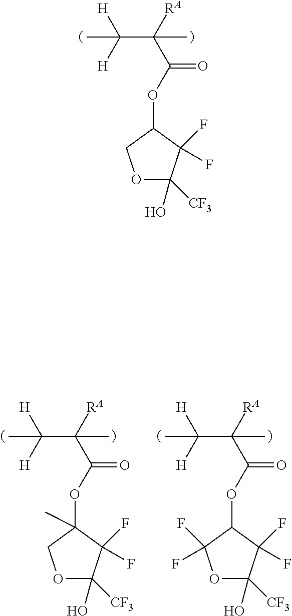

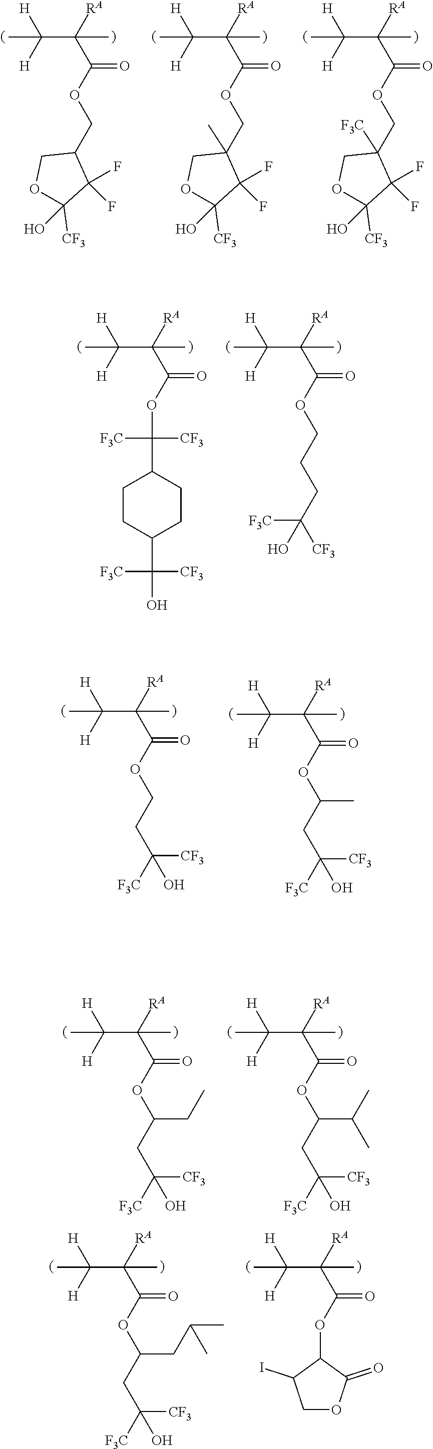

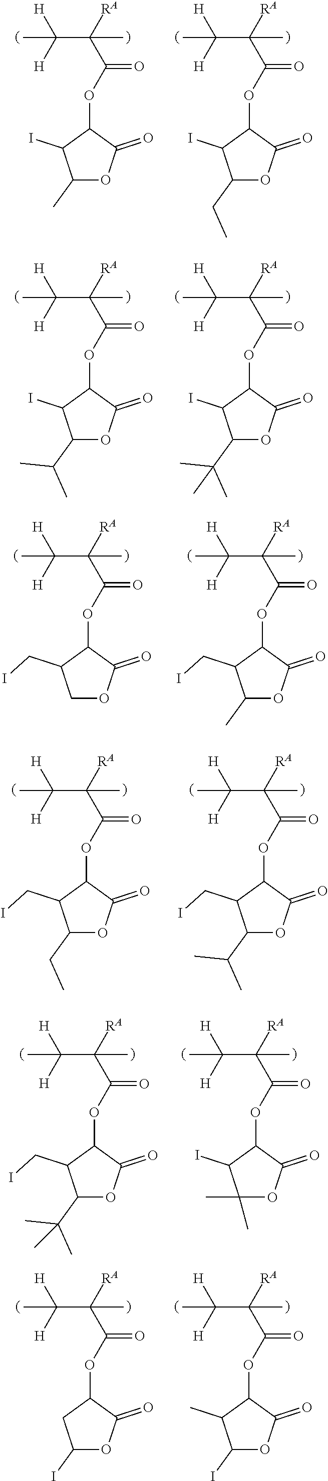

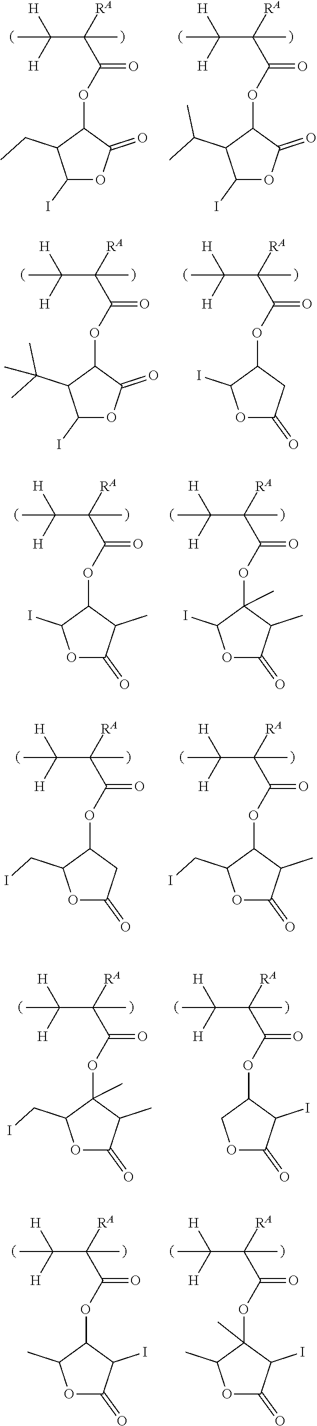

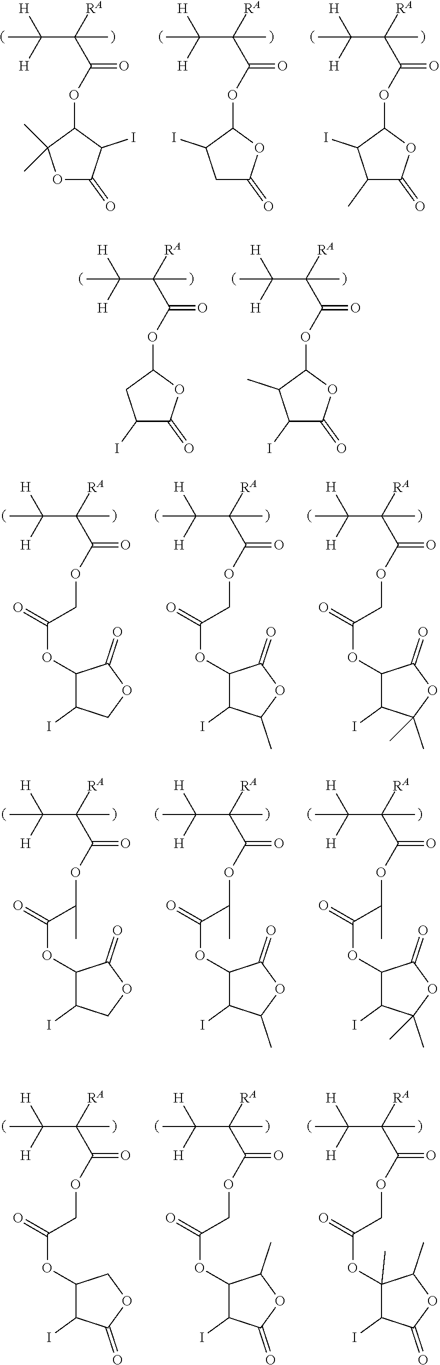

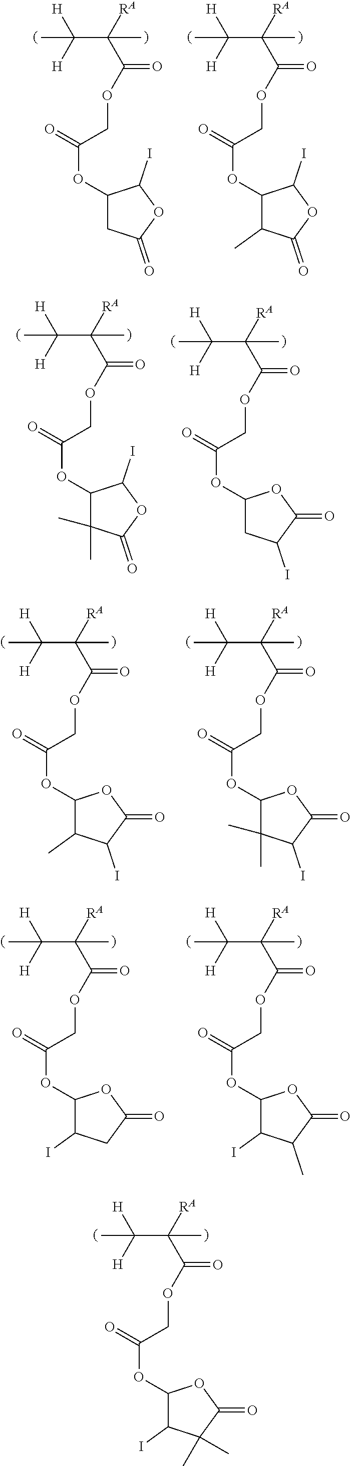

The repeating unit “d” particularly preferably has a lactone ring as a polar group in ArF-lithography, and preferably has a phenol moiety in KrF-lithography, EB-lithography, and EUV-lithography.

The polymer optionally further has a repeating unit having a structure in which a hydroxy group is protected with an acid-labile group (hereinafter, also referred to as the repeating unit “e”). The repeating unit “e” is not particularly limited as long as the repeating unit “e” has one or two or more structures in which a hydroxy group is protected, and generates the hydroxy group by decomposing the protective group by an action of an acid. The repeating unit “e” is preferably represented by the following formula (e1).

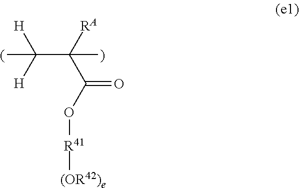

In the formula (e1), R^{A} represents a hydrogen atom, a fluorine atom, a methyl group, or a trifluoromethyl group. R⁴¹ represents a (e+1)-valent hydrocarbon group having 1 to 30 carbon atoms and optionally having a heteroatom. R⁴² represents an acid-labile group. “e” represents an integer of 1 to 4.

In the formula (e1), the acid-labile group represented by R⁴² is deprotected by an action of an acid to generate the hydroxy group. The structure of R⁴² is not particularly limited, but is preferably an acetal structure, a ketal structure, an alkoxycarbonyl group, an alkoxymethyl group represented by the following formula (e2), or the like, and particularly preferably an alkoxymethyl group represented by the following formula (e2).

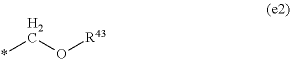

In the formula, “*” represents a bond. R⁴³ represents a hydrocarbyl group having 1 to 15 carbon atoms.

Specific examples of the acid-labile group represented by R⁴², the alkoxymethyl group represented by the formula (e2), and the repeating unit “e” includes groups same as those exemplified in the description of the repeating unit d described in JP2020-111564 A.

The polymer may further have, as a unit for adjusting solubility of the polymer, a repeating unit “f” derived from styrene, vinylnaphthalene, or a methacrylate substituted with a linear, branched, or cyclic hydrocarbon group, such as methyl methacrylate, ethyl methacrylate, n-butyl methacrylate, isopropyl methacrylate, cyclohexane methacrylate, and 1-adamantane methacrylate, and a derivative thereof.

The polymer optionally further has a repeating unit “g” derived from indene, benzofuran, benzothiophene, acenaphthylene, chromone, coumarin, norbornadiene, or a derivative thereof. Specific examples of a monomer to yield the repeating unit “g” include the following monomers, but the monomer is not limited thereto.

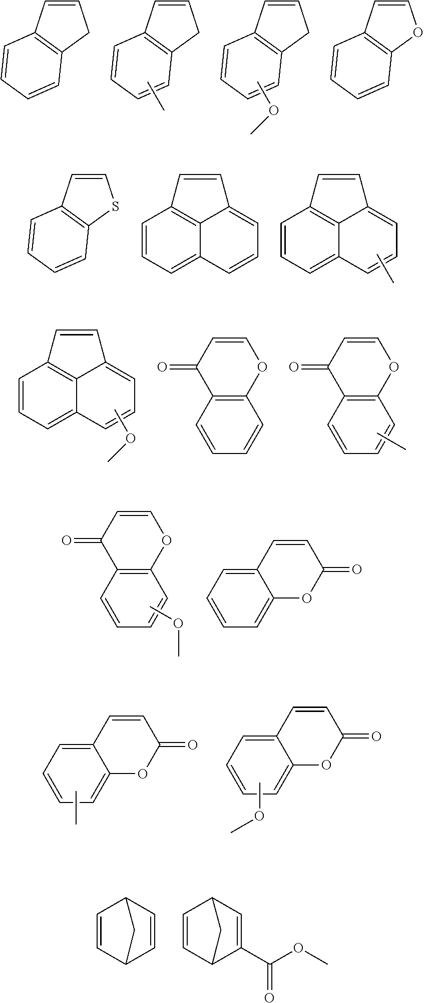

The polymer in the present invention may further have a repeating unit “h” that generates an acid when exposed to light. The repeating unit “h” preferably have a structure represented by the following formulae (h1) to (h4).

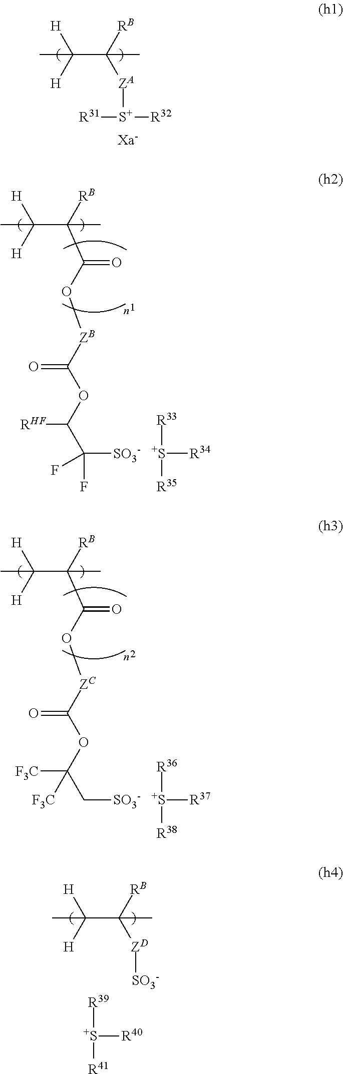

In the formulae (h1) to (h4), R^{B} represents a hydrogen atom, a fluorine atom, a methyl group or a trifluoromethyl group. Z^{A} represents a single bond, a phenylene group, “—O—Z^{A}—”, “—C(═O)—O—Z^{A1}—”, or “—C(═O)—NH—Z^{A1}—”. Z^{A1} represents a hydrocarbylene group having 1 to 20 carbon atoms and optionally having a heteroatom. Z^{B} and Z^{C} represent each independently a single bond or a hydrocarbylene group having 1 to 20 carbon atoms and optionally having a heteroatom. Z^{D} represents a single bond, a methylene group, an ethylene group, a phenylene group, a fluorinated phenylene group, “—O—Z^{D1}—”, “—C(═O)—O—Z^{D1}”, or “—C(═O)—NH—Z^{D1}—”. Z^{D1} represents an optionally substituted phenylene group.

The hydrocarbylene group represented by Z^{A1} may be saturated or unsaturated, and may be linear, branched, or cyclic. Examples thereof include: alkanediyl groups such as a methylene group, an ethane-1,1-diyl group, an ethane-1,2-diyl group, a propane-1,2-diyl group, a propane-2,2-diyl group, a propane-1,3-diyl group, a 2-methylpropane-1,3-diyl group, a butane-1,3-diyl group, a butane-2,3-diyl group, a butane-1,4-diyl group, a pentane-1,3-diyl group, pentane-1,4-diyl group, a 2,2-dimethylpropane-1,3-diyl group, a pentane-1,5-diyl group, and a hexane-1,6-diyl group; cyclic saturated hydrocarbylene groups such as a cyclopentane-1,2-diyl group, a cyclopentane-1,3-diyl group, a cyclohexane-1,6-diyl group, and an adamantane-1,3-diyl group; alkenediyl groups such as an ethene-1,2-diyl group, a 1-propene-1,3-diyl group, a 2-butene-1,4-diyl group, and a 1-methyl-1-butene-1,4-diyl group; cyclic unsaturated aliphatic hydrocarbylene groups such as 2-cyclohexene-1,4-diyl group; aromatic hydrocarbylene groups such as a phenylene group; and groups obtained by combining these groups. Further, some of the hydrogen atoms of these group may be substituted with a group having a heteroatom such as an oxygen atom, a sulfur atom, a nitrogen atom, and a halogen atom (particularly, fluorine atom or iodine atom), and some of carbon atoms of these groups may be substituted with a group having a heteroatom such as an oxygen atom, a sulfur atom, and a nitrogen atom. As a result, the hydrocarbylene groups may have a hydroxy group, a cyano group, a carbonyl group, an ether bond, an ester bond, a sulfonate ester bond, a carbonate bond, a lactone ring, a sultone ring, a carboxylic acid anhydride, a haloalkyl group, etc.

The hydrocarbylene group represented by Z^{B} and Z^{C} may be saturated or unsaturated, and may be linear, branched, or cyclic. Specific examples thereof include the same as those exemplified as the aliphatic hydrocarbylene group represented by Z^{A1}. Z^{B} and Z^{C} are preferably an adamantyl group or a substituted phenylene group.

In the formulae (h1) to (h4), R³¹ to R⁴¹ are each independently a hydrocarbyl group having 1 to 20 carbon atoms and optionally having a heteroatom. The hydrocarbyl group may be saturated or unsaturated, and may be linear, branched, or cyclic. The specific examples include: alkyl groups such as a methyl group, an ethyl group, an n-propyl group, an isopropyl group, an n-butyl group, and a tert-butyl group; cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups such as a vinyl group, a allyl group, a propenyl group, a butenyl group, and a hexenyl group; cyclic unsaturated aliphatic hydrocarbyl groups such as a cyclohexenyl group; aryl groups such as a phenyl group and a naphthyl group; heteroaryl groups such as a thienyl group; and aralkyl groups such as a benzyl group, a 1-phenylethyl group, and a 2-phenylethyl group. Among these, an aryl group is preferable. Further, some of the hydrogen atoms of these group may be substituted with a group having a heteroatom such as an oxygen atom, a sulfur atom, a nitrogen atom, and a halogen atom, and some of carbon atoms of these groups may be substituted with a group having a heteroatom such as an oxygen atom, a sulfur atom, and a nitrogen atom. As a result, the hydrocarbyl groups may have a hydroxy group, a cyano group, a carbonyl group, an ether bond, an ester bond, a sulfonate ester bond, a carbonate bond, a lactone ring, a sultone ring, a carboxylic acid anhydride, a haloalkyl group, etc.

Z^{A} and R³¹ to R⁴¹ each preferably have a phenyl group that is bonded to S⁺ in the formula as a structure.

In addition, any two of Z^{A}, R³¹, and R³² may be bonded to each other to form a ring together with the sulfur atom to which they are bonded, and any two of R³³, R³⁴, and R³⁵, any two of R³⁶, R³⁷, and R³⁸, or any two of R³⁹, R⁴⁰, and R⁴¹ may be bonded to each other to form a ring together with the sulfur atom to which they are bonded.

In the formula (h2), R^{HF} is a hydrogen atom or a trifluoromethyl group.

In the formula (h2), n¹ is 0 or 1, and n¹ is 0 when Z^{B} is a single bond. In the formula (h3), n² is 0 or 1, and n² is 0 when Z^{C} is a single bond.

In the formula (h1), Xa⁻ represents a non-nucleophilic counterion. The non-nucleophilic counterion is not particularly limited, but the examples include: halide ions, such as a chloride ion and a bromide ion; fluoroalkylsulfonate ions, such as a triflate ion, a 1,1,1-trifluoroethanesulfonate ion, and a nonafluorobutanesulfonate ion; arylsulfonate ions, such as a tosylate ion, a benzenesulfonate ion, a 4-fluorobenzenesulfonate ion, and a 1,2,3,4,5-pentafluorobenzenesulfonate ion; alkylsulfonate ions, such as a mesylate ion and a butanesulfonate ion; imide ions, such as a bis(trifluoromethylsulfonyl)imide ion, a bis(perfluoroethylsulfonyl)imide ion, and a bis(perfluorobutylsulfonyl)imide ion; and methide ions, such as a tris(trifluoromethylsulfonyl)methide ion and a tris(perfluoroethylsulfonyl)methide ion. The non-nucleophilic counterion is preferably anions represented by the following formulae (h1-1) to (h1-3).

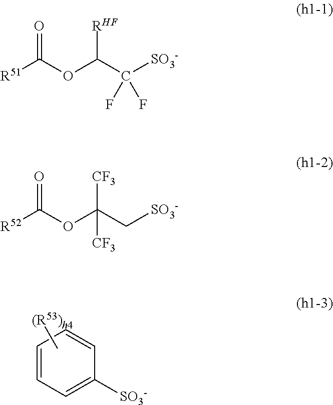

In the formulae (h1-1) to (h1-2), R⁵¹ and R⁵² represent each independently a hydrocarbyl group having 1 to 40 carbon atoms and optionally having a heteroatom. R^{HF} represents a hydrogen atom or a trifluoromethyl group. In the formula (h1-3), R⁵³ represents a hydroxyl group, a halogen atom, or a hydrocarbyl group having 1 to 40 carbon atoms and optionally having a heteroatom. h4 represents an integer of 0 to 5.

Examples of the anion represented by formula (h1-1) include those described in paragraphs [0100] to [0101] of JP2014-177407A and those represented by the following formulae, but are not limited thereto. In the following formulae, R^{H}F is the same as defined above.

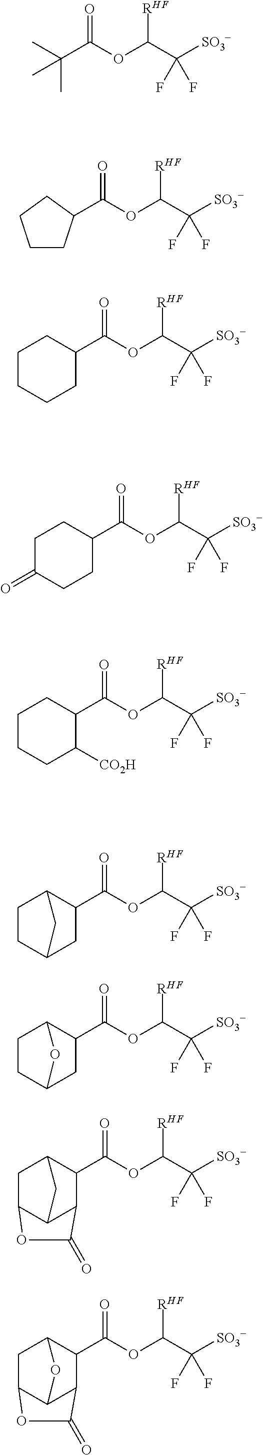

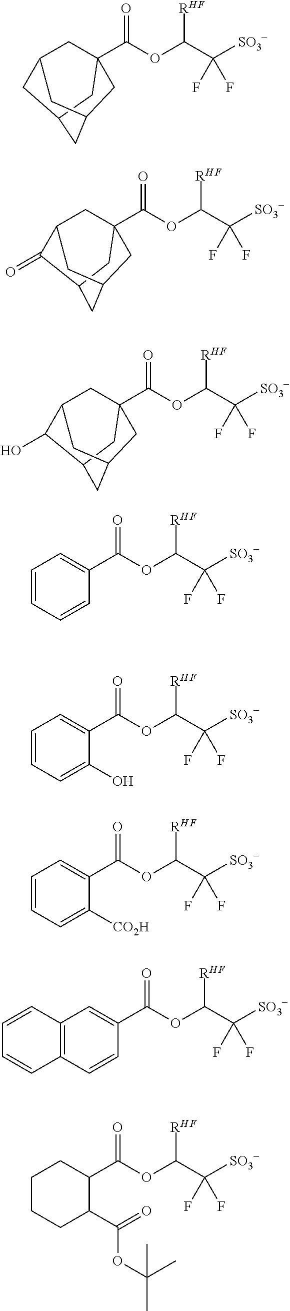

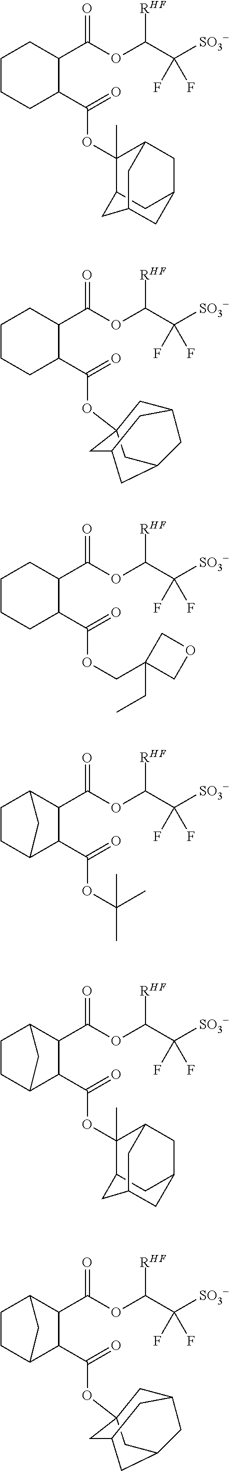

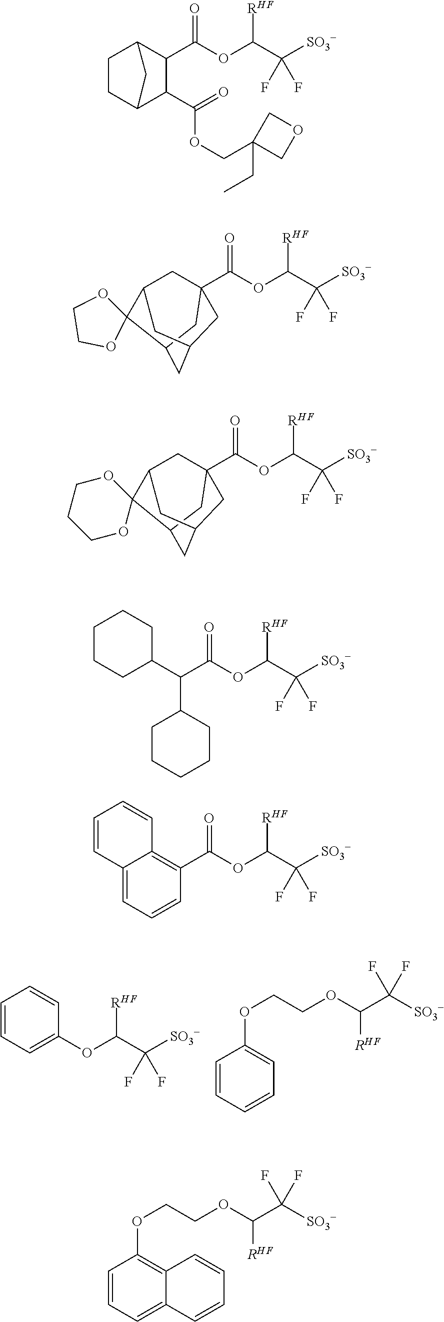

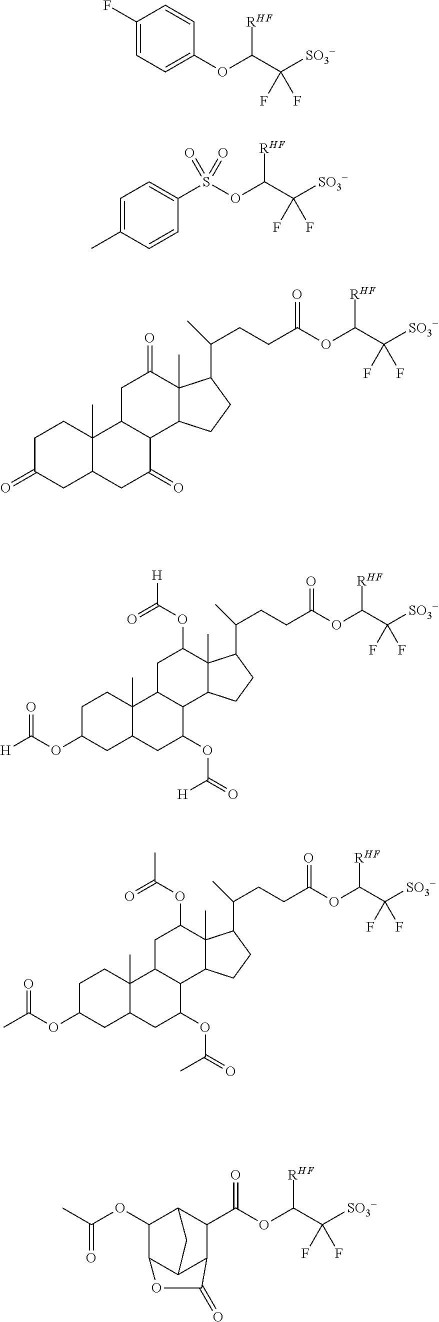

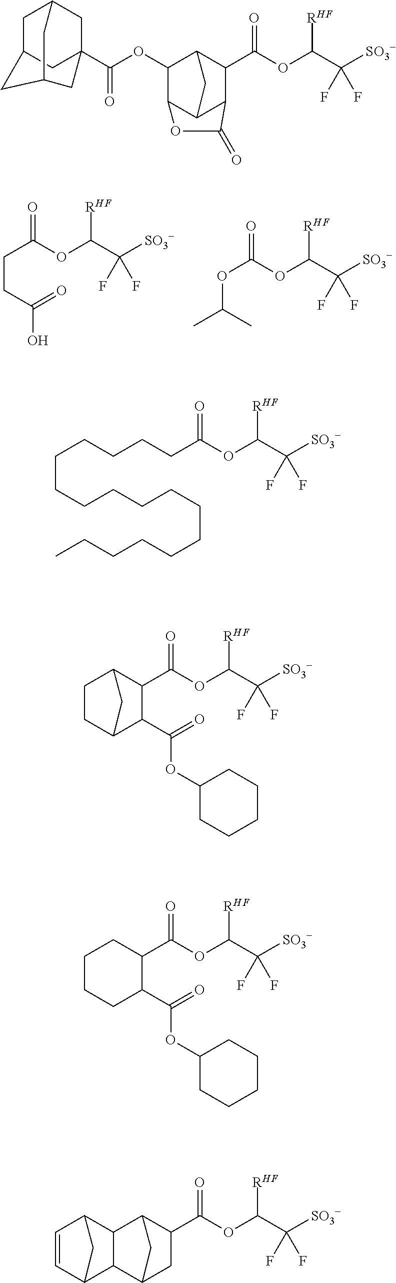

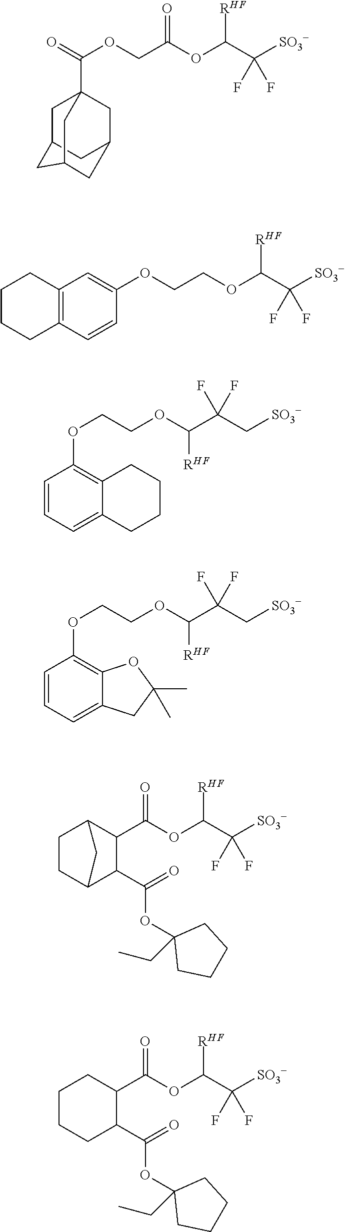

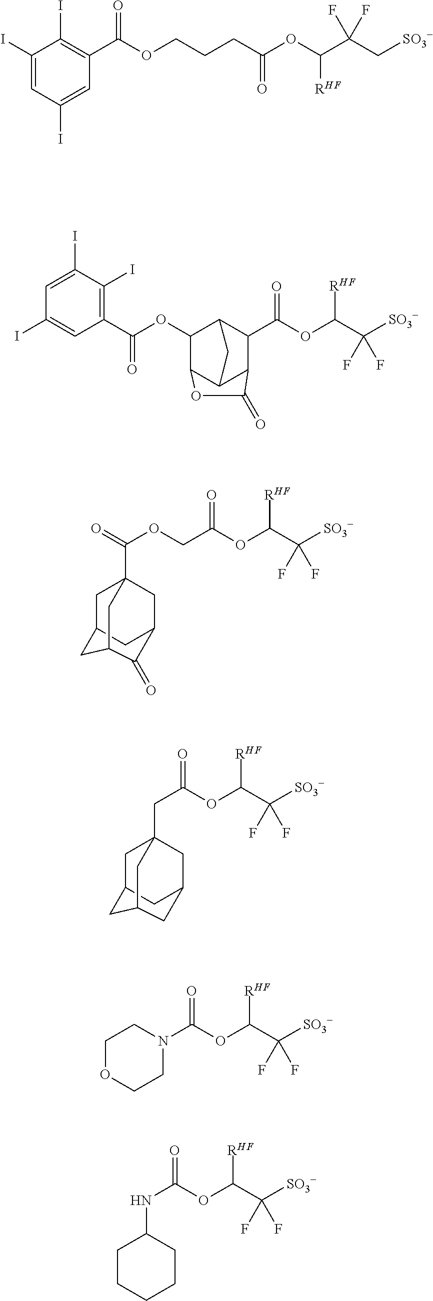

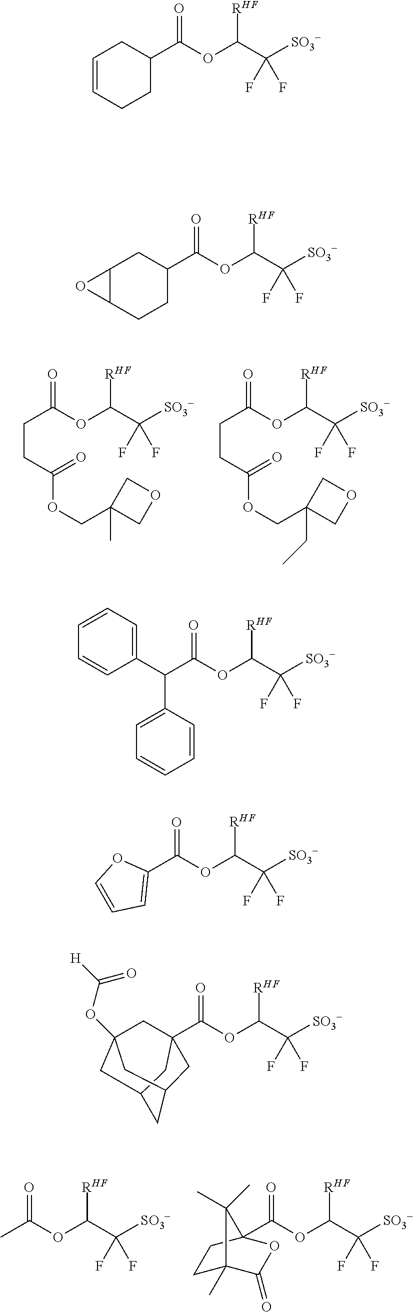

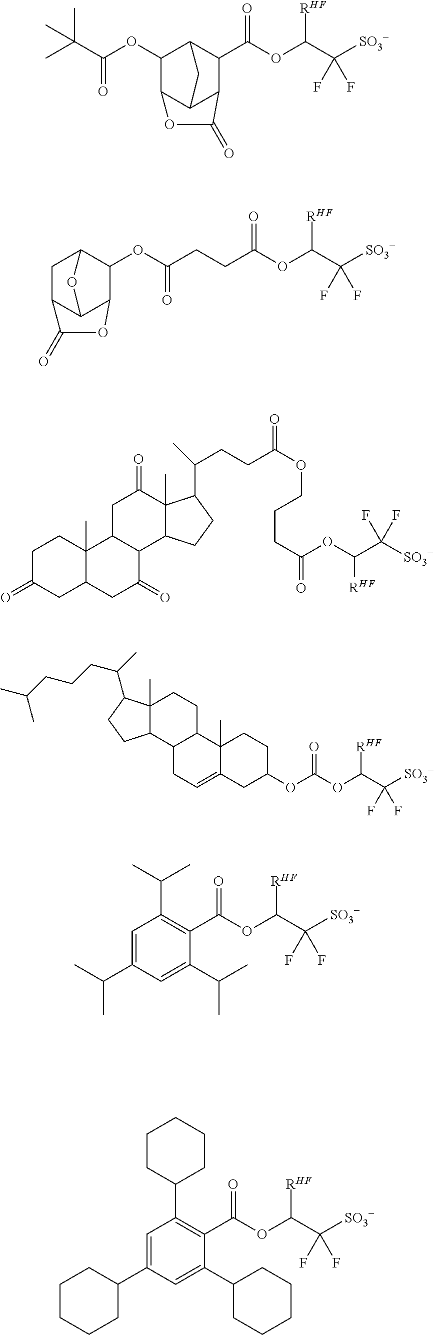

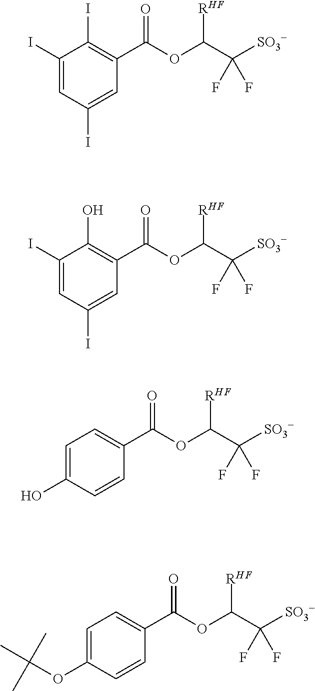

Examples of the anion represented by formula (h1-2) include those described in paragraphs [0080] to [0081] of JP2010-215608A and those represented by the following formulae, but are not limited thereto. In the following formulae, Ac is an acetyl group.

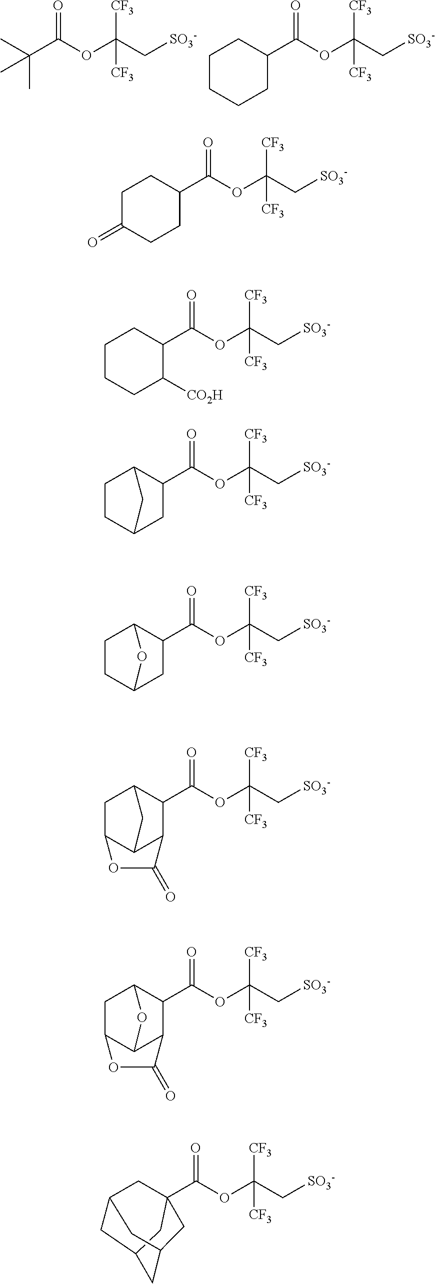

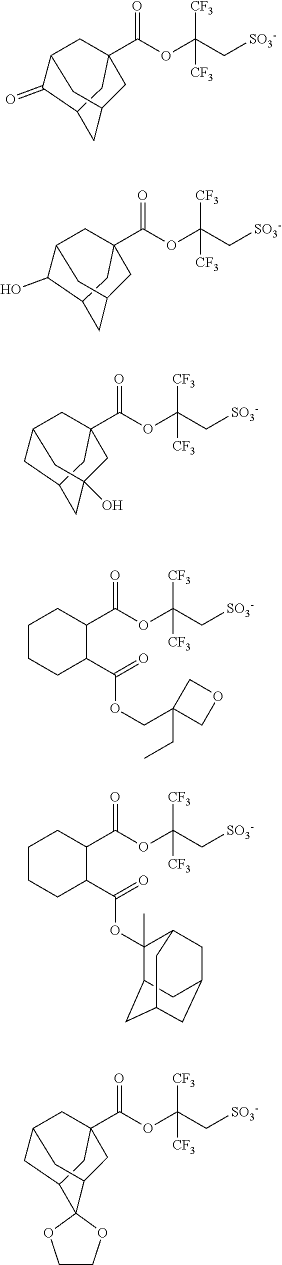

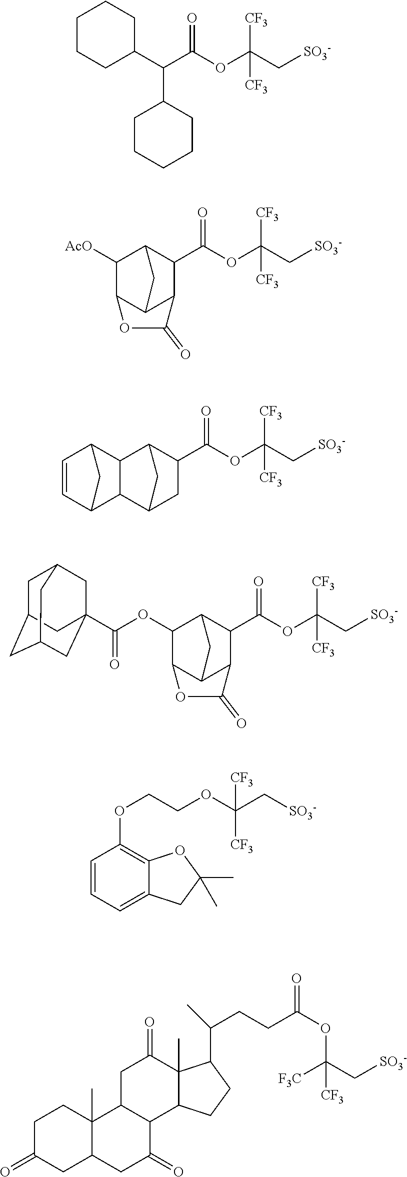

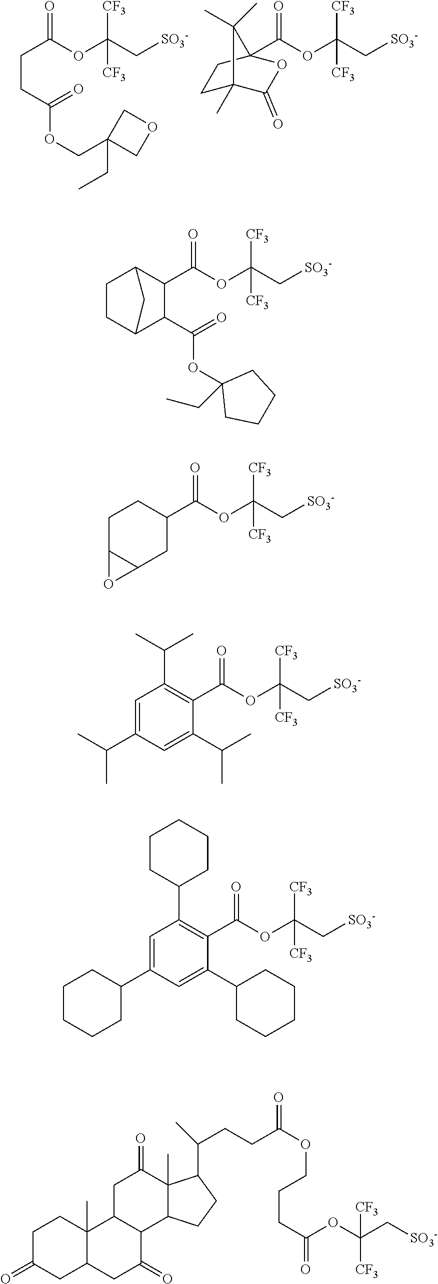

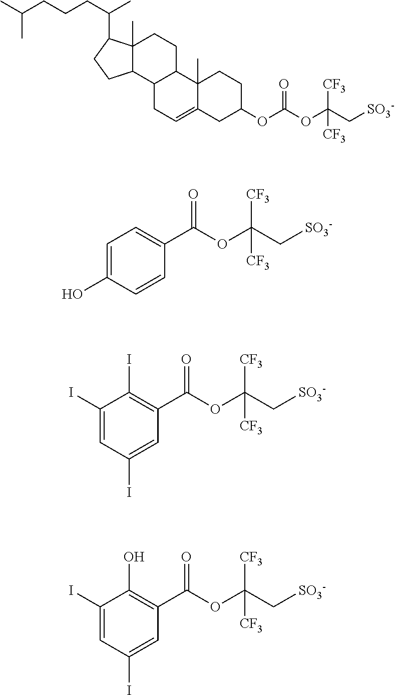

Examples of the anion represented by formula (h1-3) include those represented by the following formulae, but are not limited thereto.

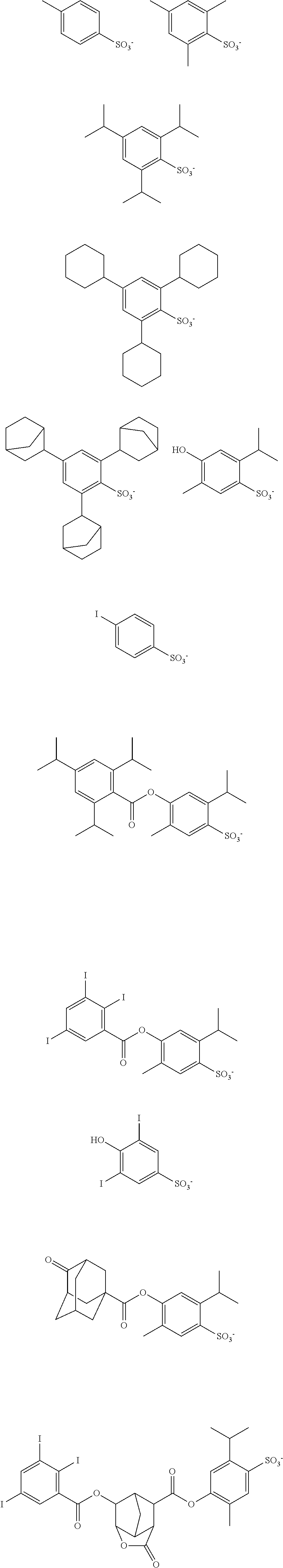

Examples of the anion in the repeating unit h2 include those described in paragraphs [0021] to [0026] of JP2014-177407A. Specific examples of the anion structure in which R^{HF} is a hydrogen atom include those described in paragraphs [0021] to [0028] of JP2010-116550A, and specific examples of the anion structure in which R^{HF} is a trifluoromethyl group include those described in paragraphs [0021] to [0027] of JP2010-77404A.

The anions in the repeating unit h3 include an anion in which the “—CH(R^{HF})CF₂SO₃—” moiety in the specific examples of the anion in the repeating unit h2 is substituted with “—C(CF₃)₂CH₂SO₃—”.

Preferable examples of the anion of the repeating units h2 to h4 include those shown below, but are not limited thereto. In the following formulae, R^{B} is the same as defined above.

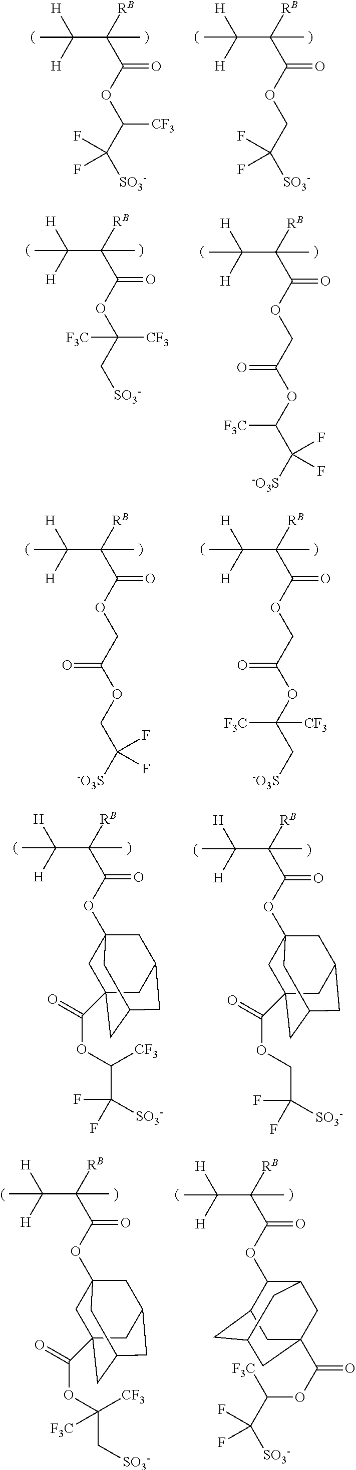

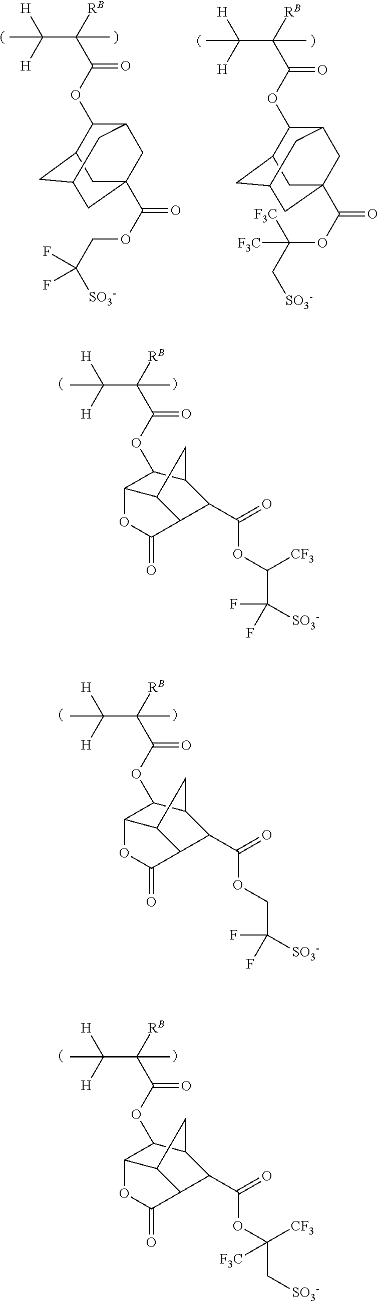

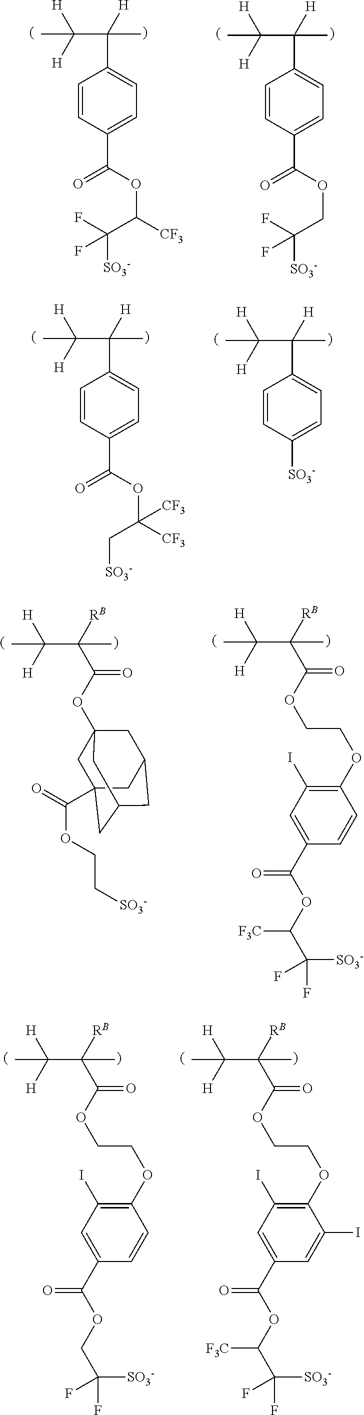

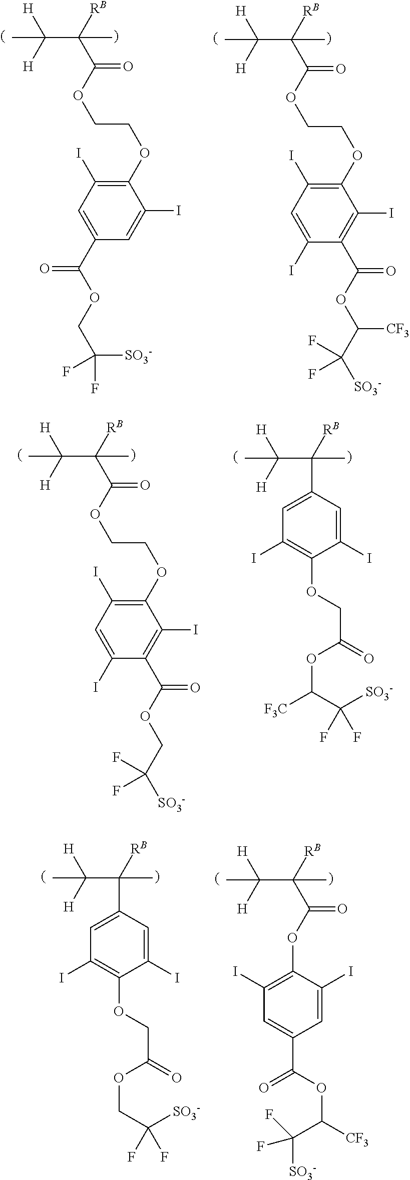

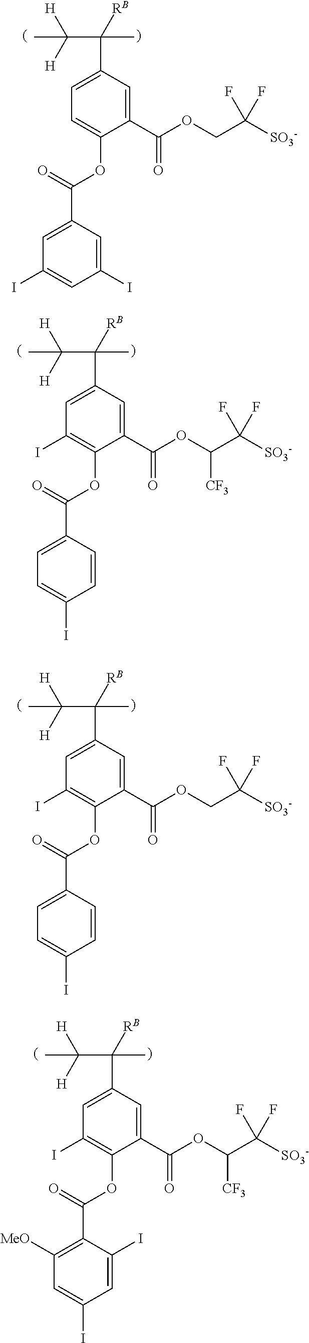

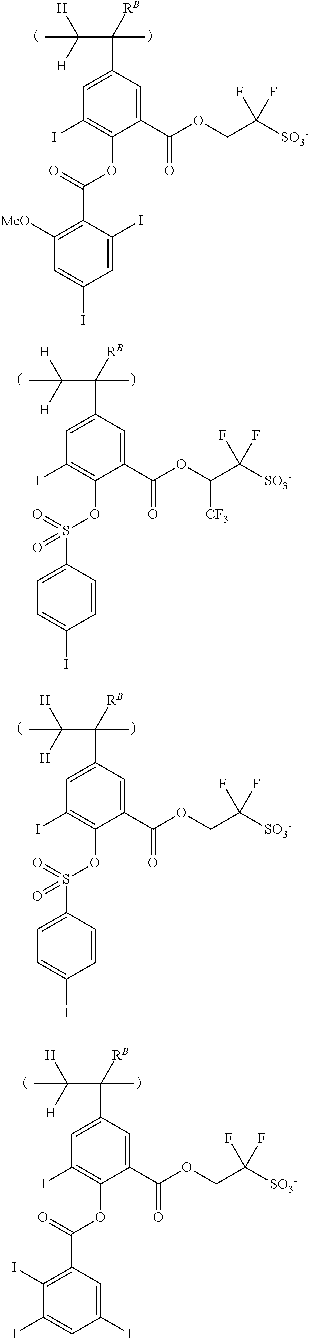

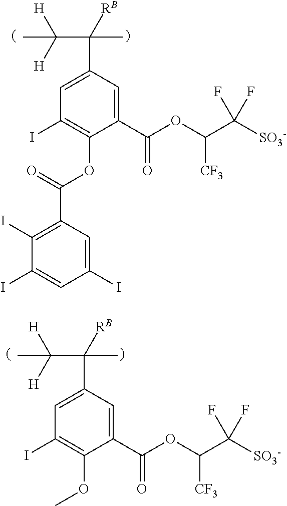

Specific examples of the sulfonium cation structure in the repeating units h2 to h4 include those described in paragraph [0223] of JP2008-158339A and those shown below, but are not limited thereto. In the following formulae, Me is a methyl group, and tBu is a tert-butyl group.

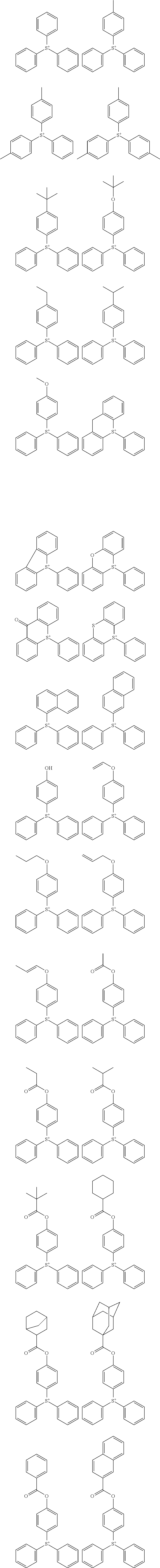

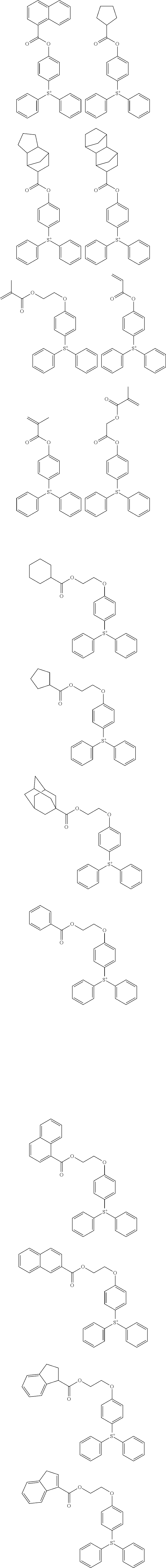

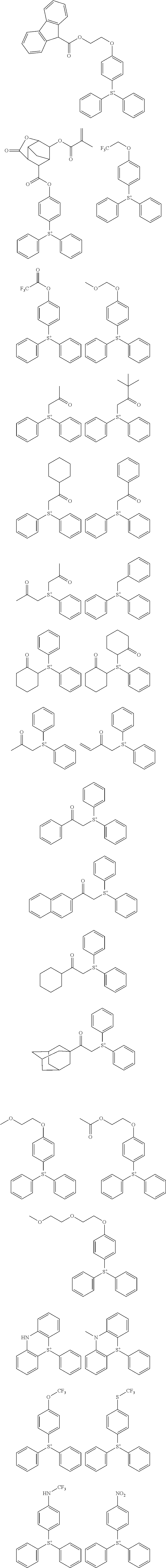

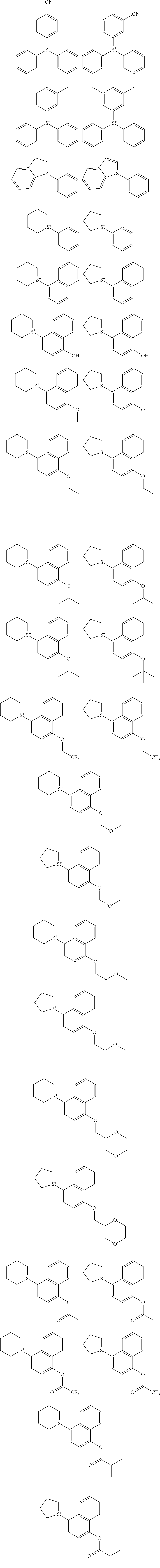

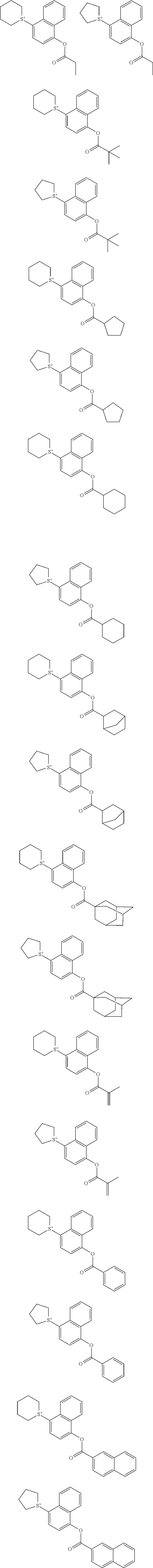

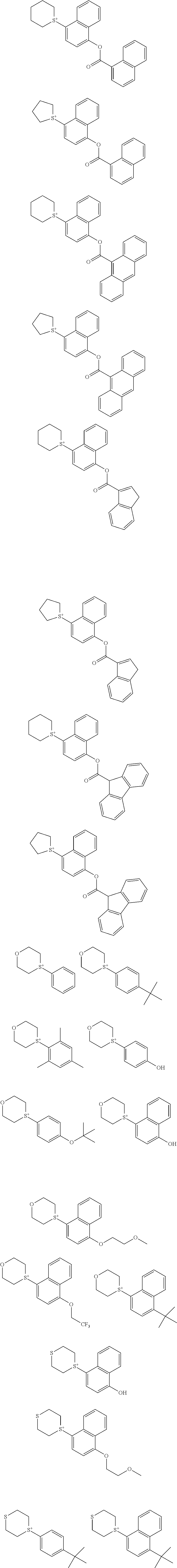

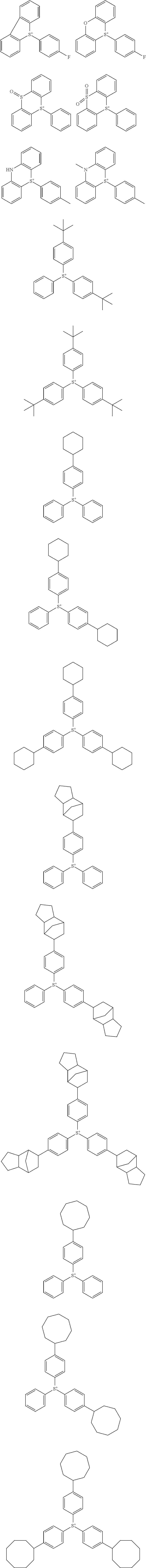

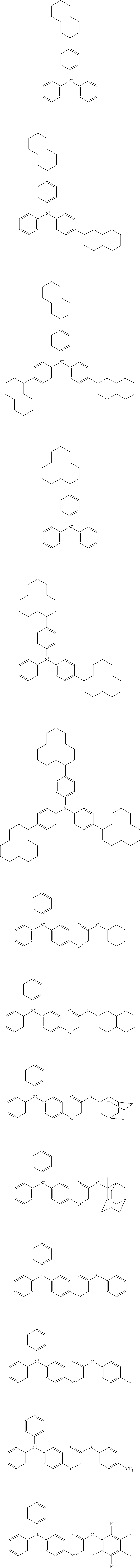

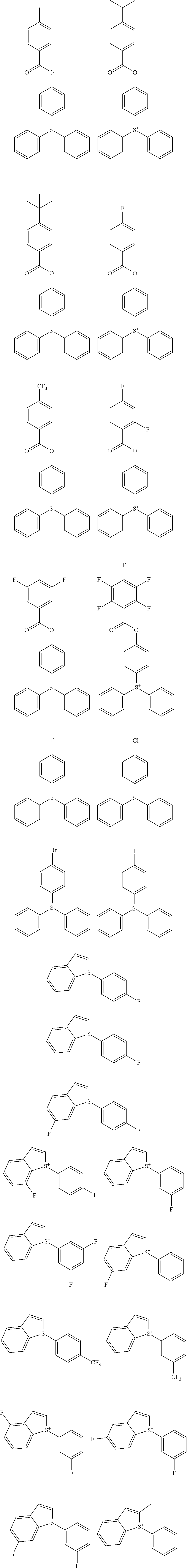

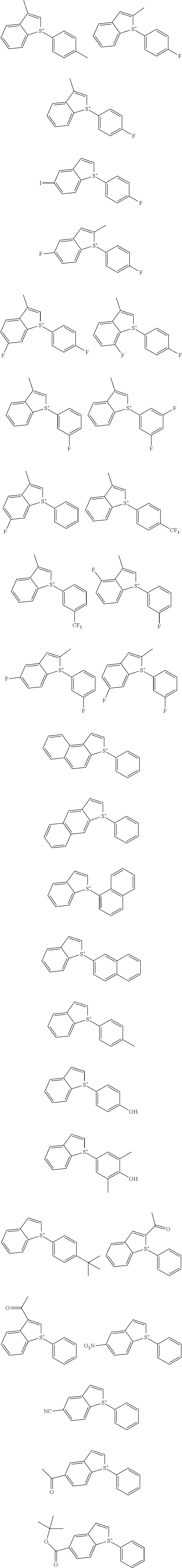

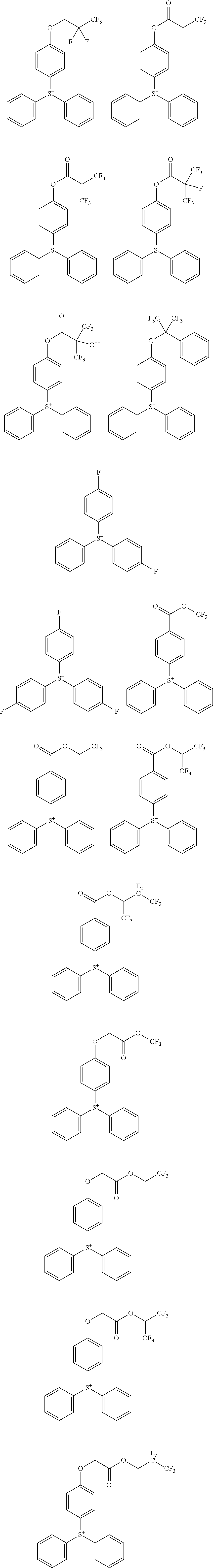

[chemical structure]

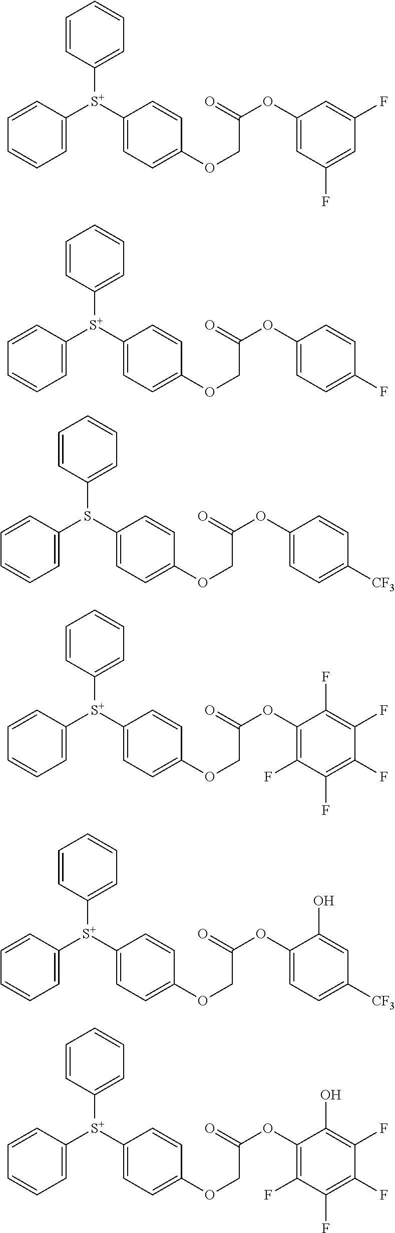

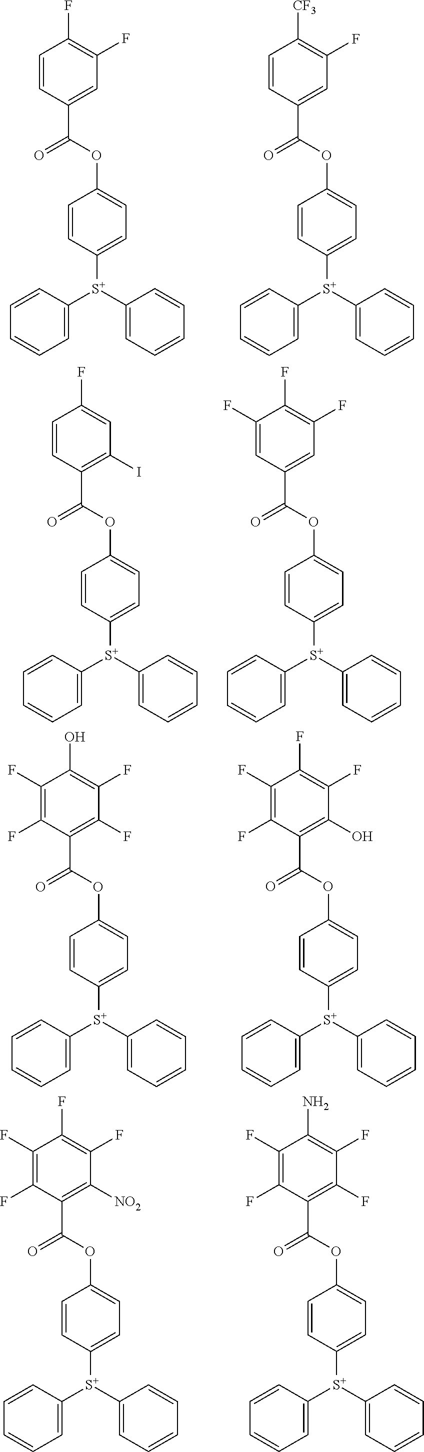

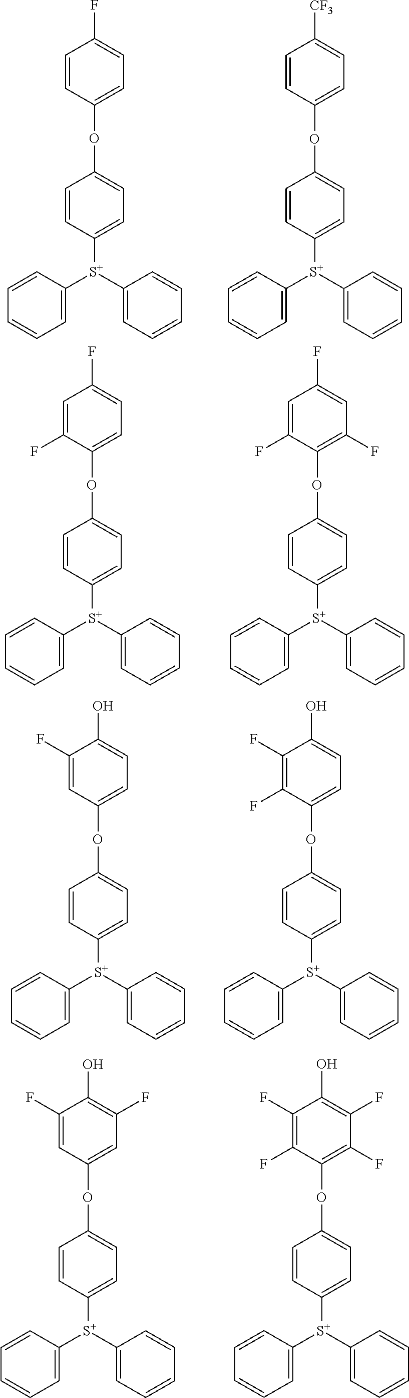

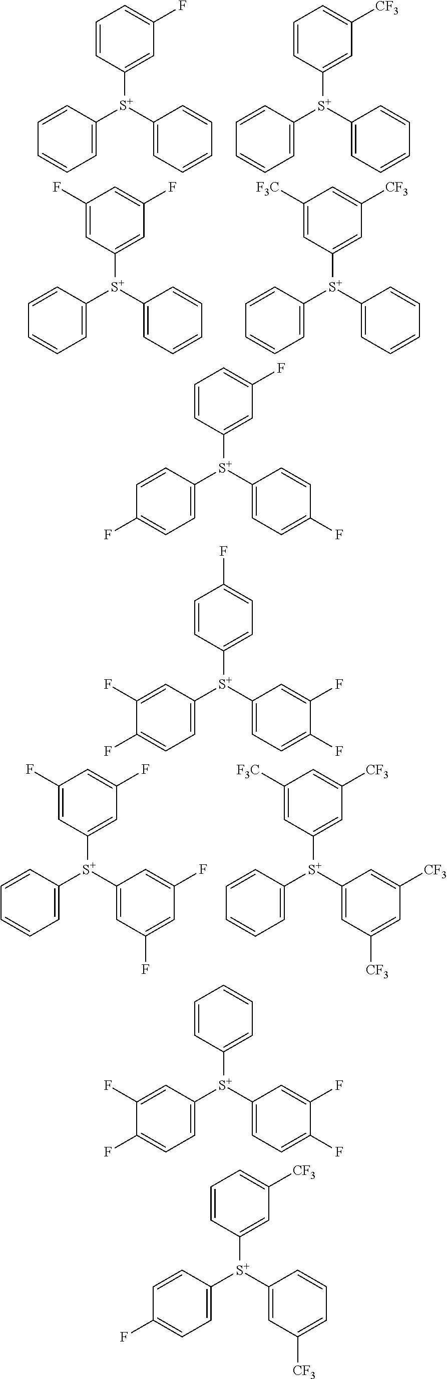

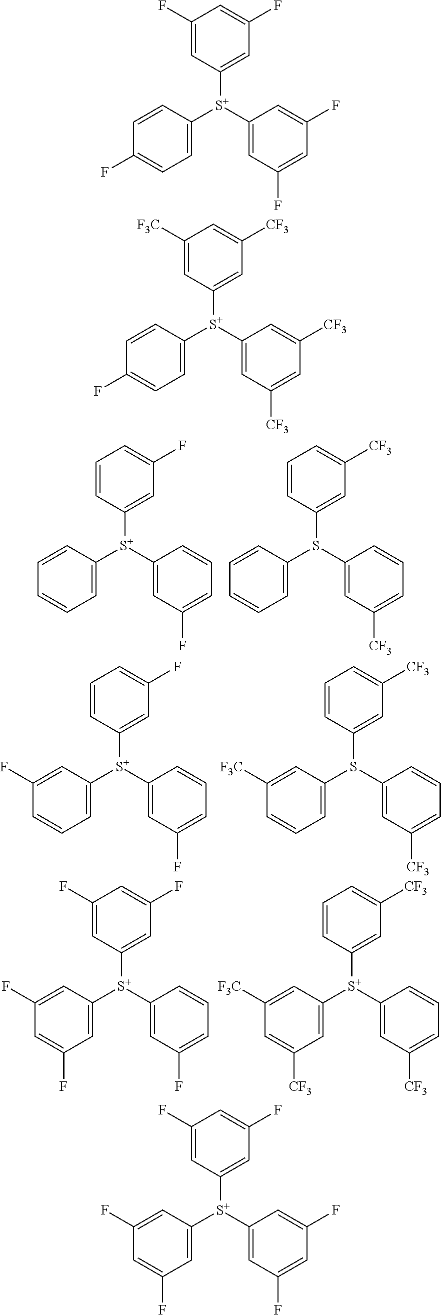

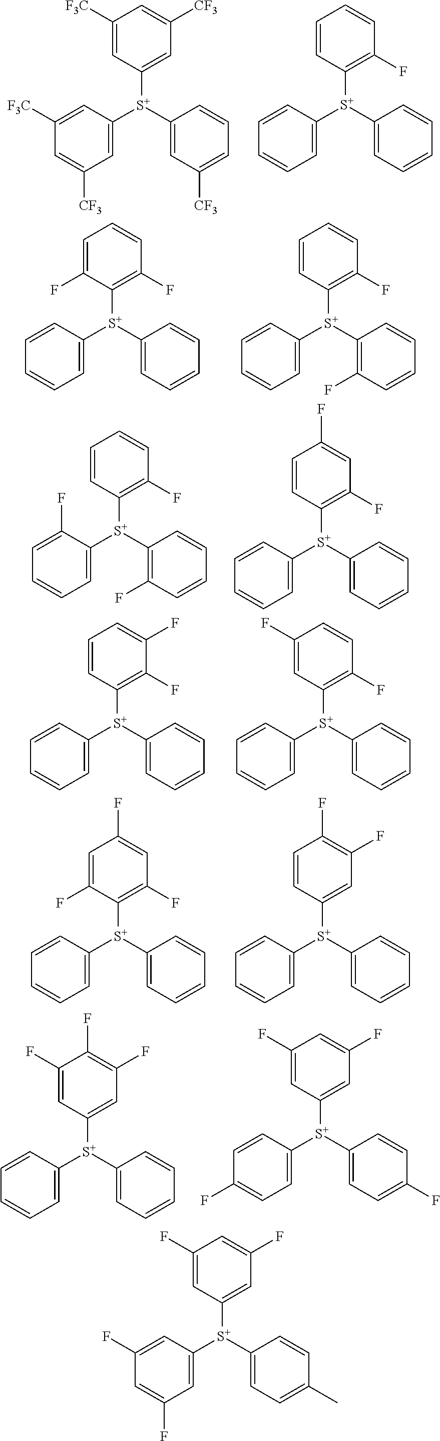

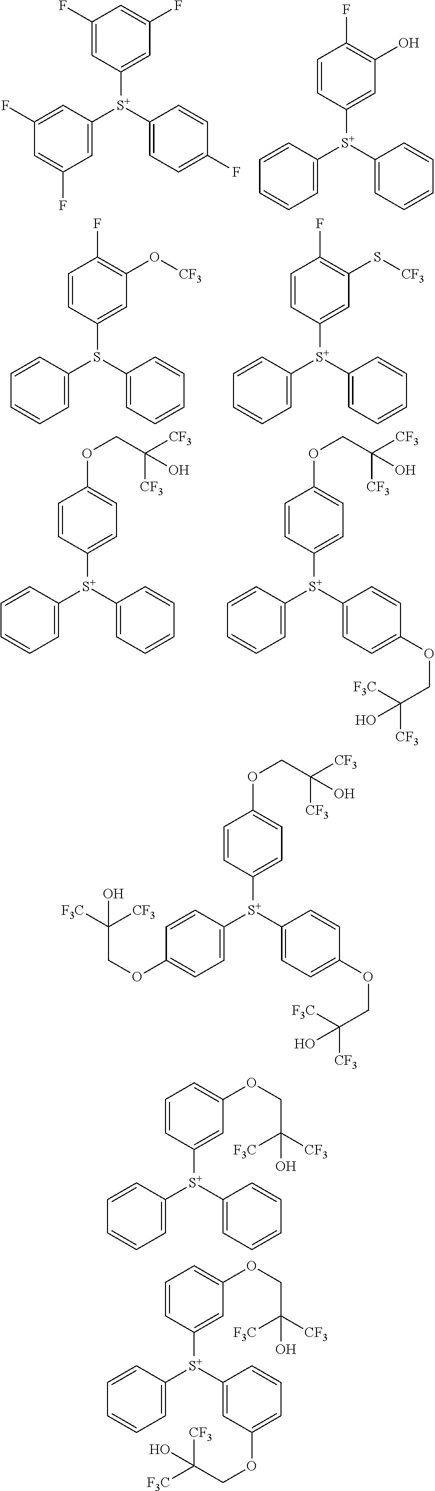

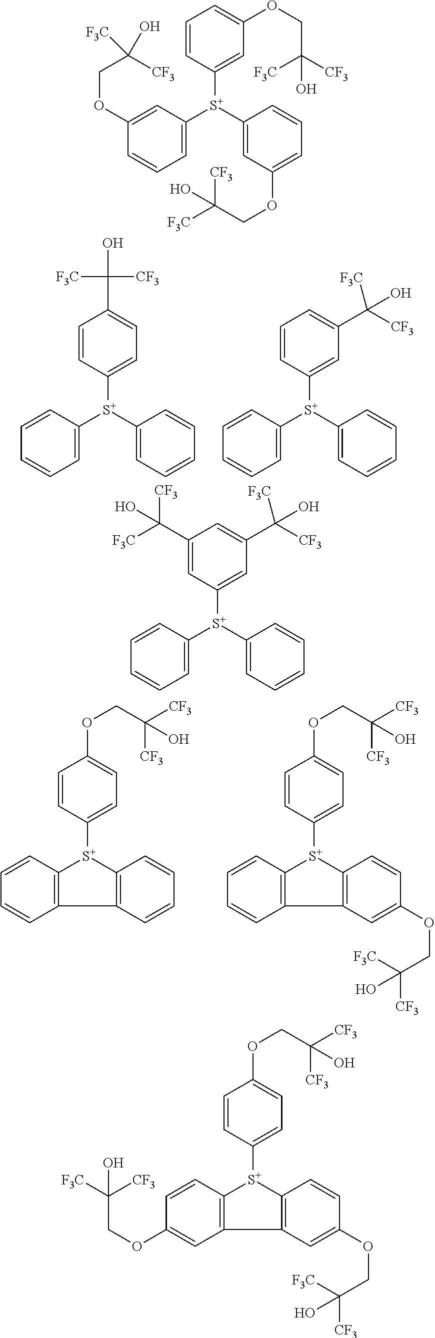

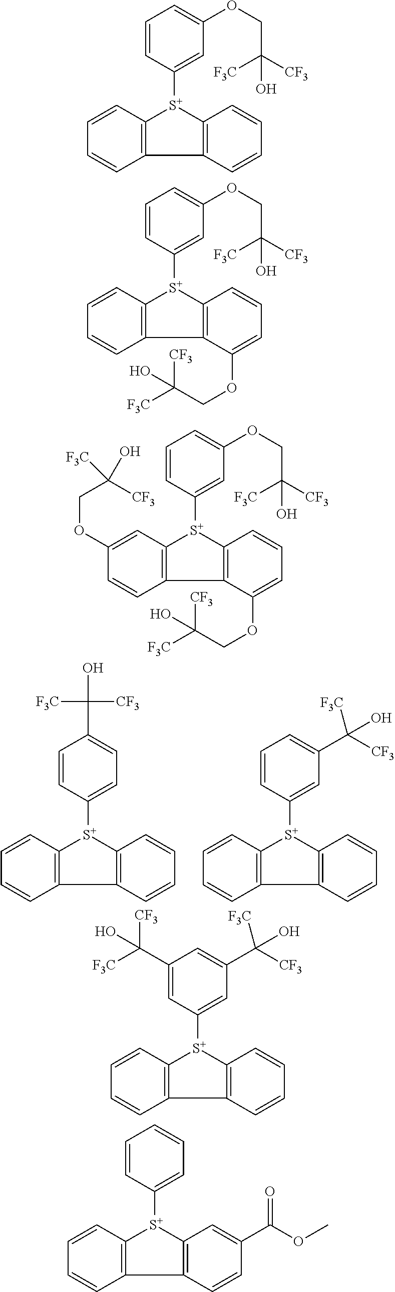

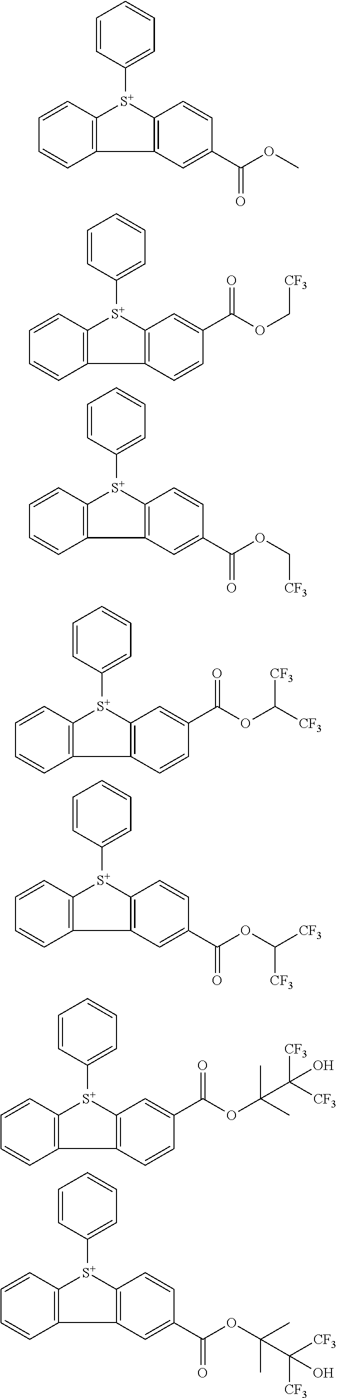

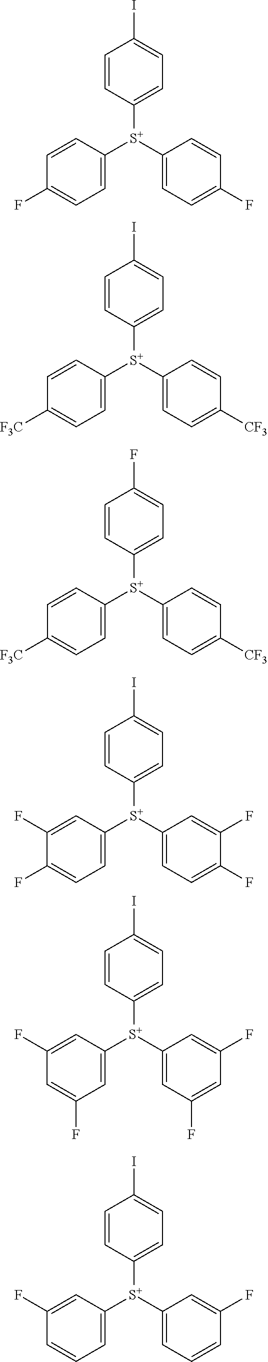

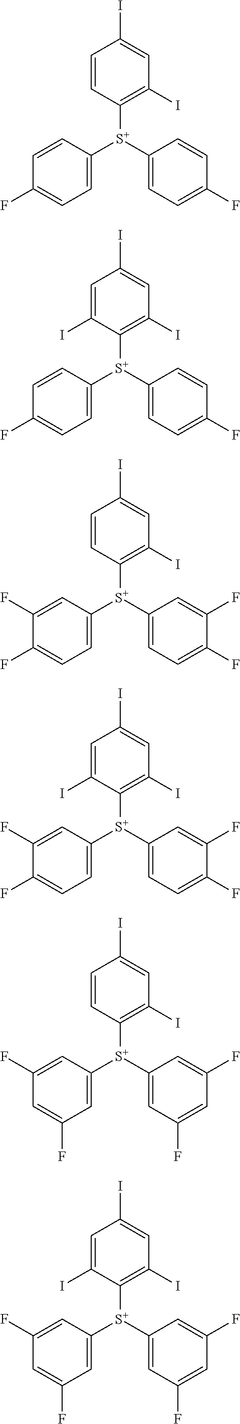

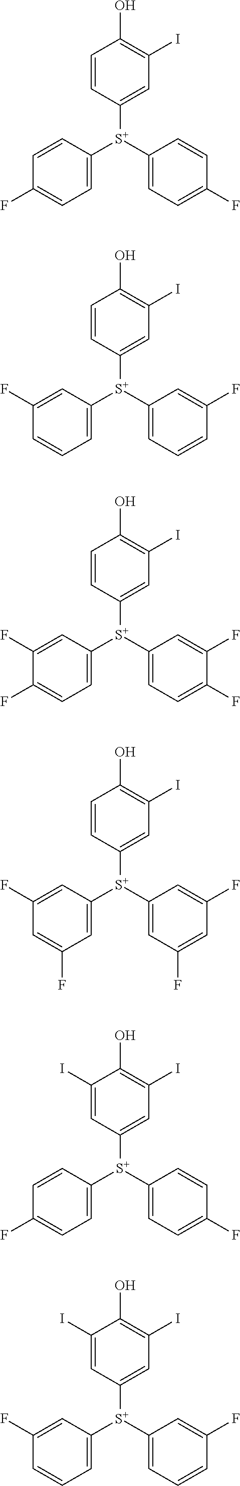

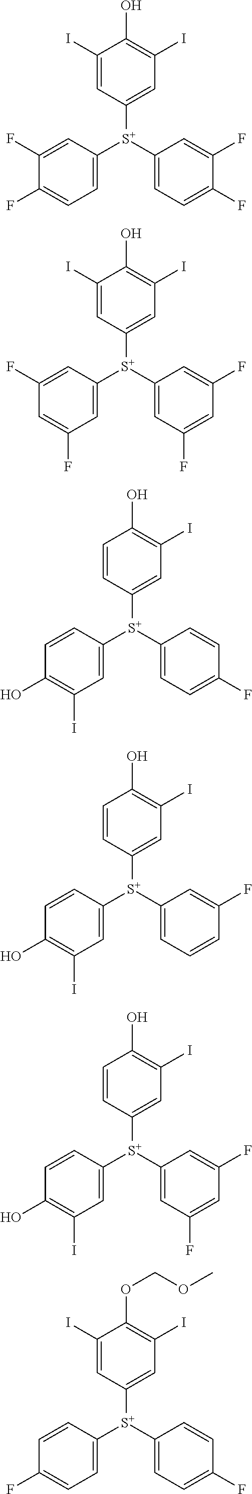

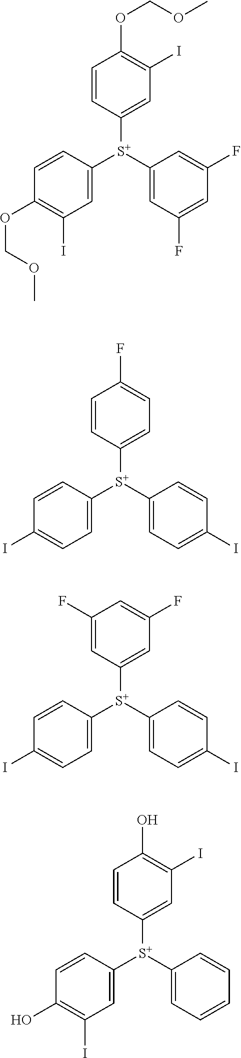

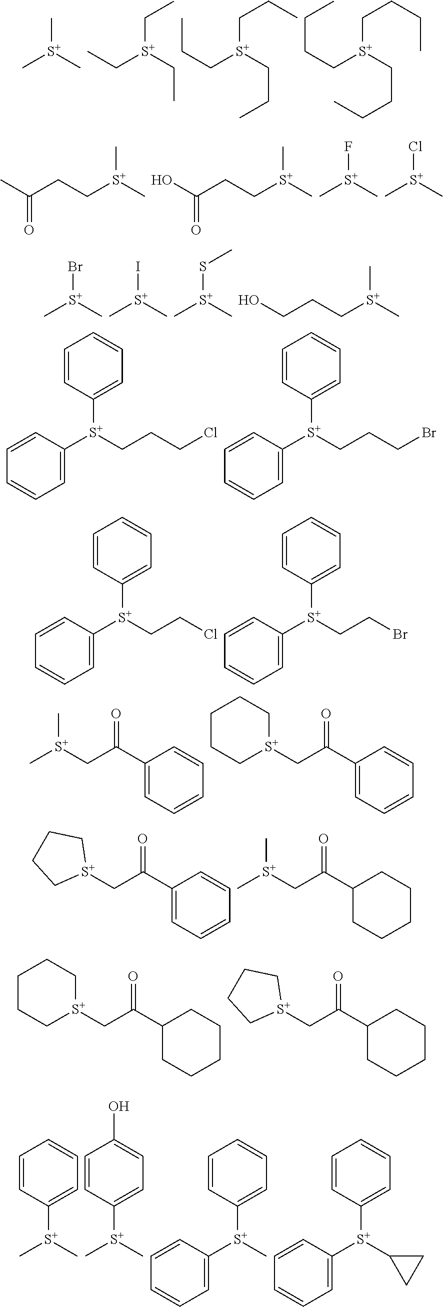

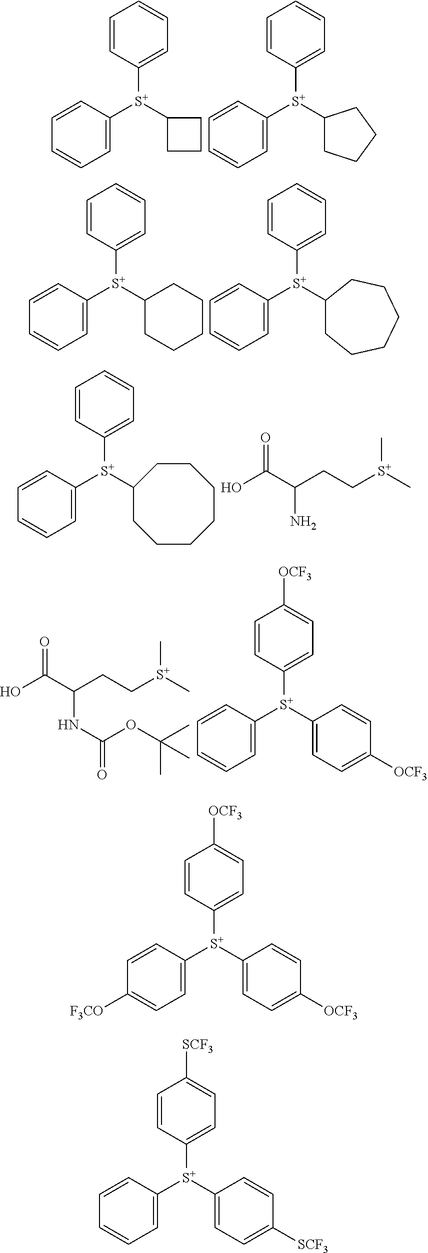

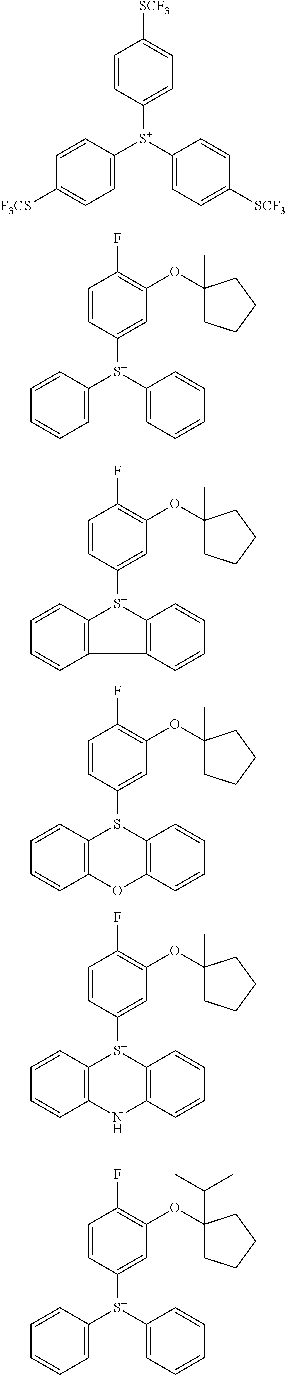

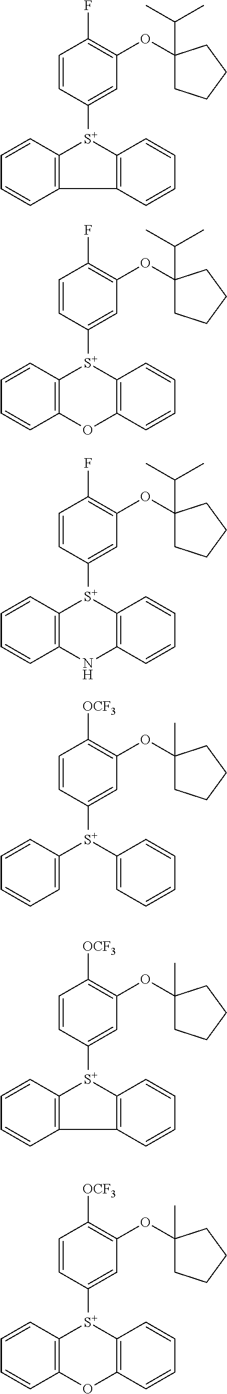

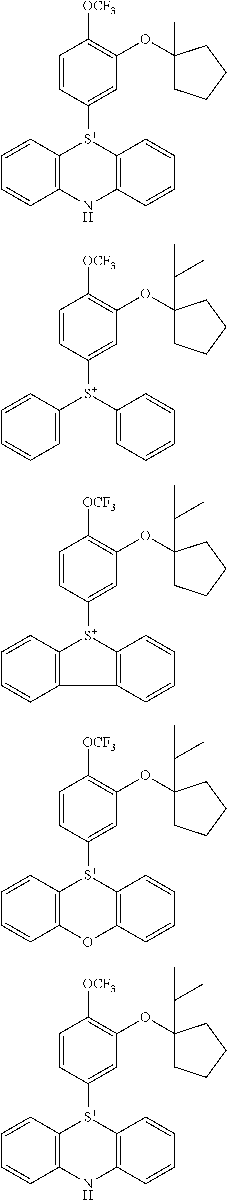

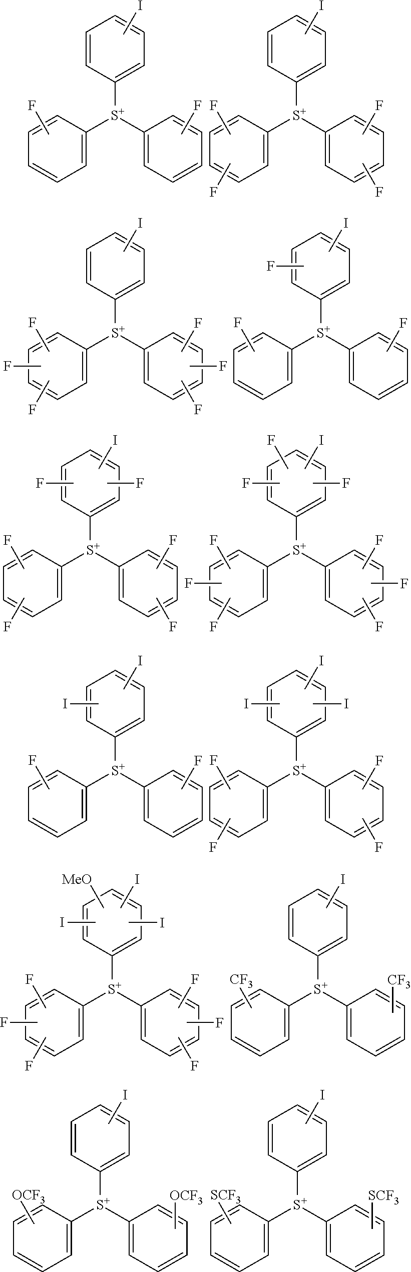

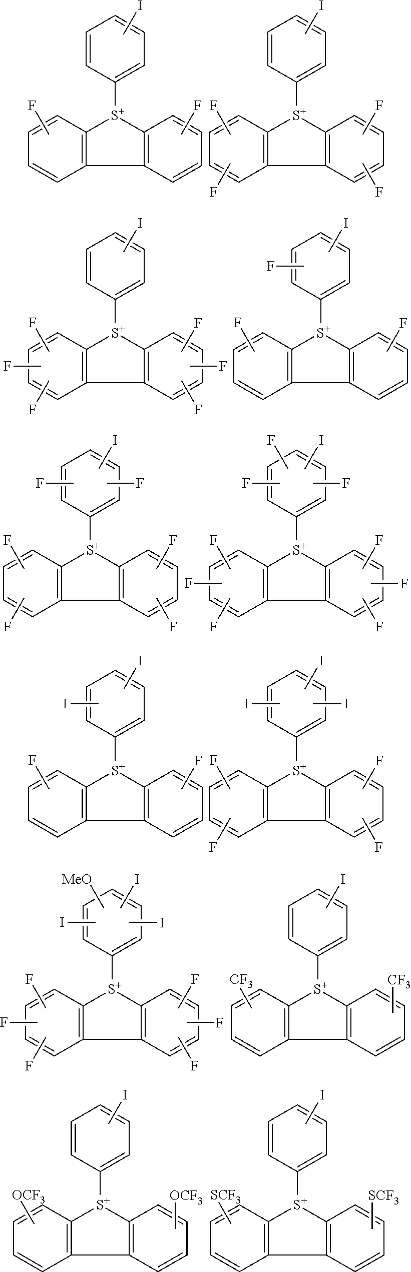

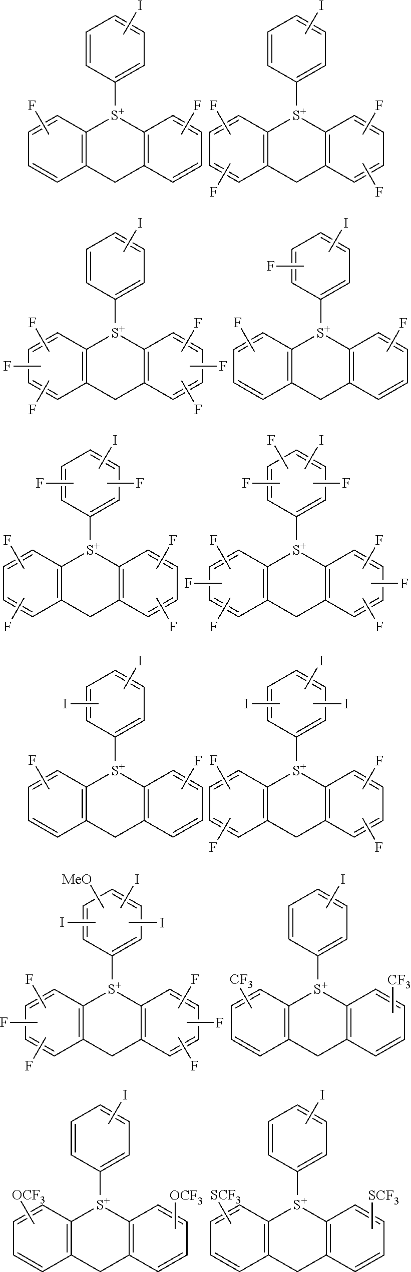

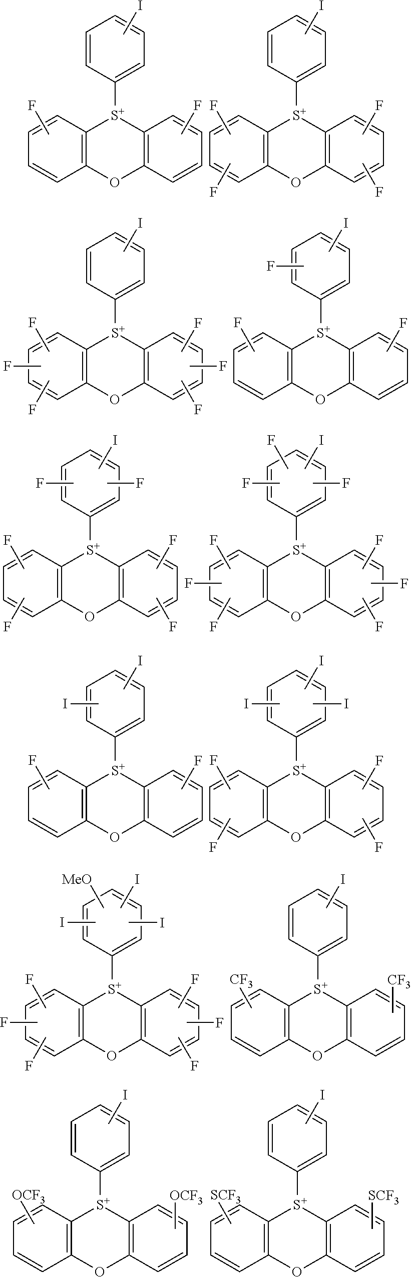

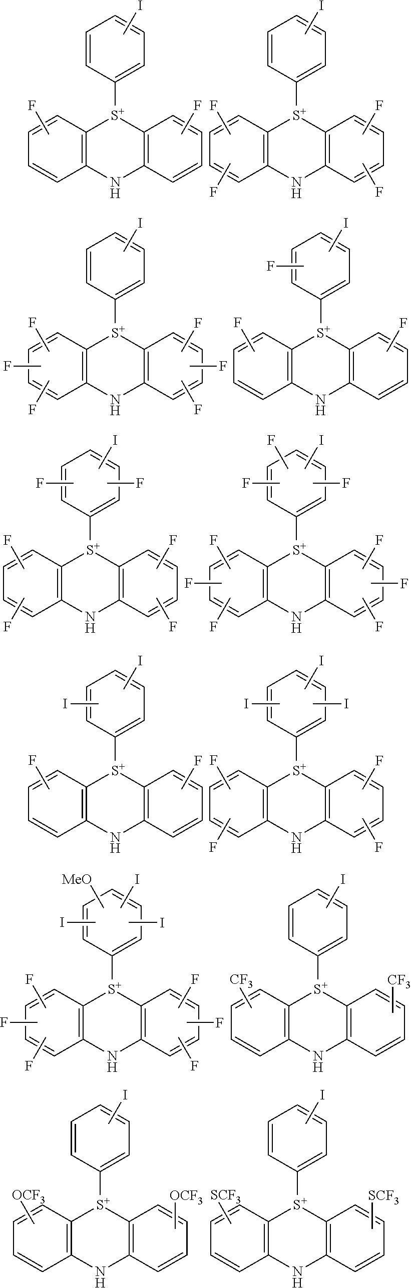

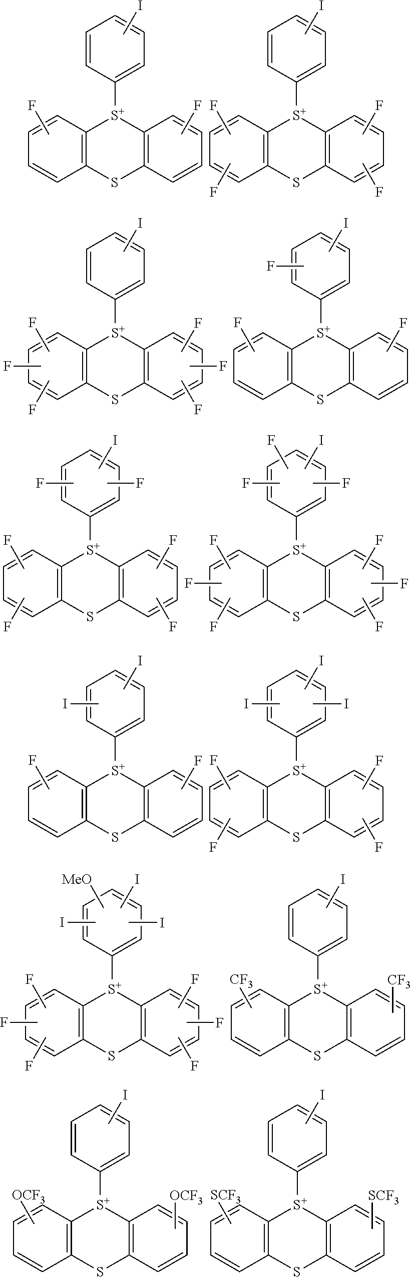

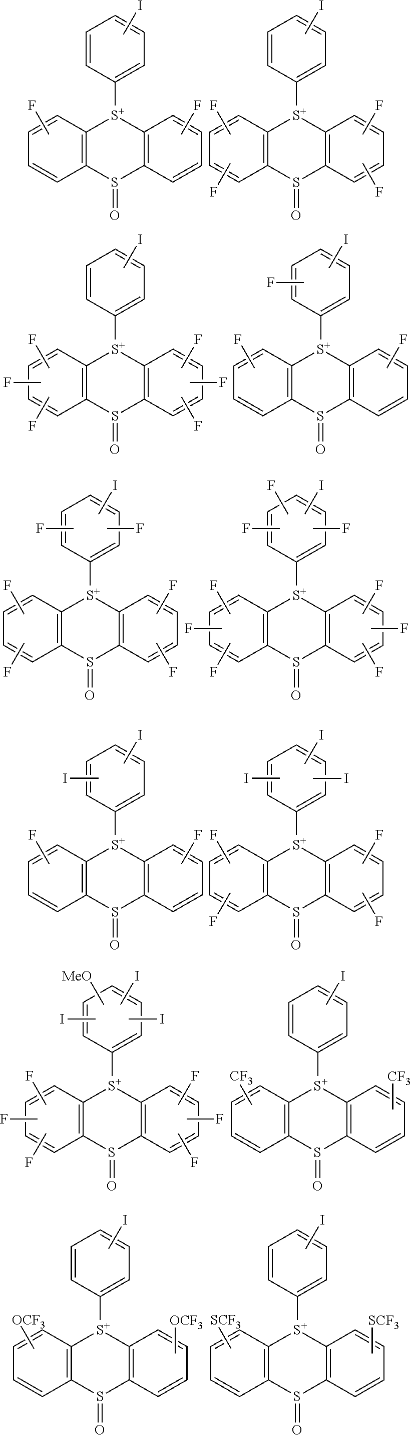

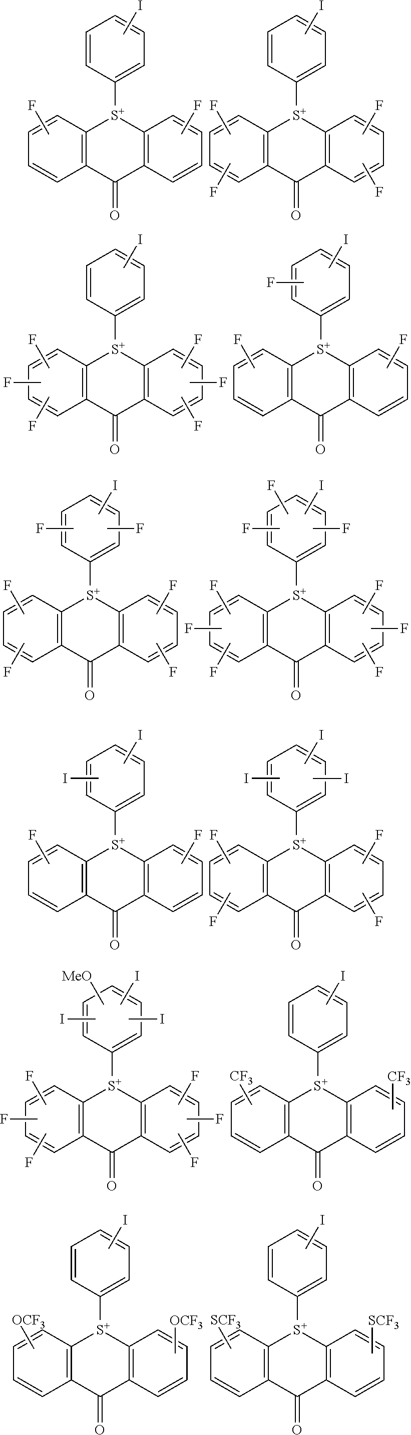

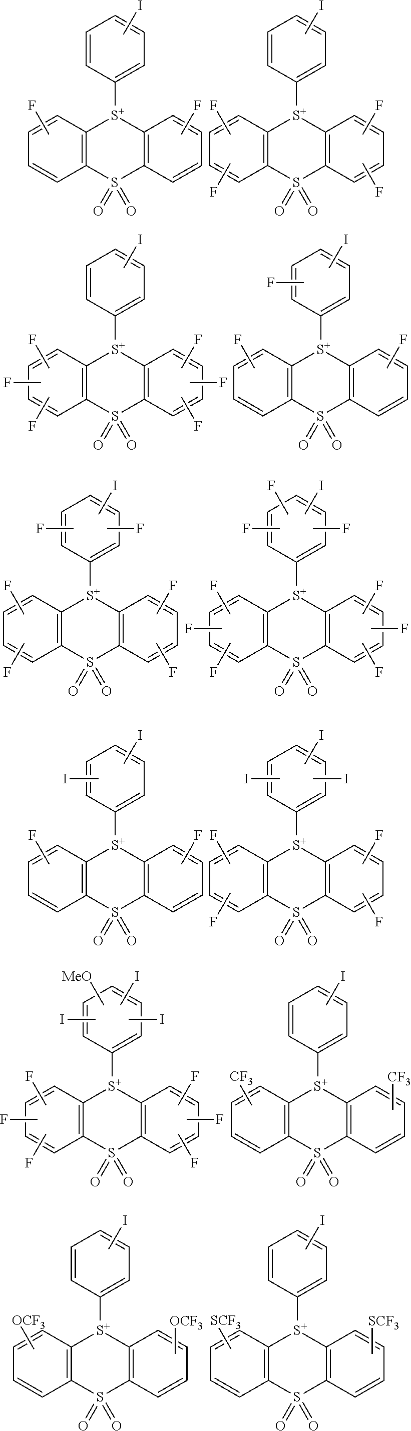

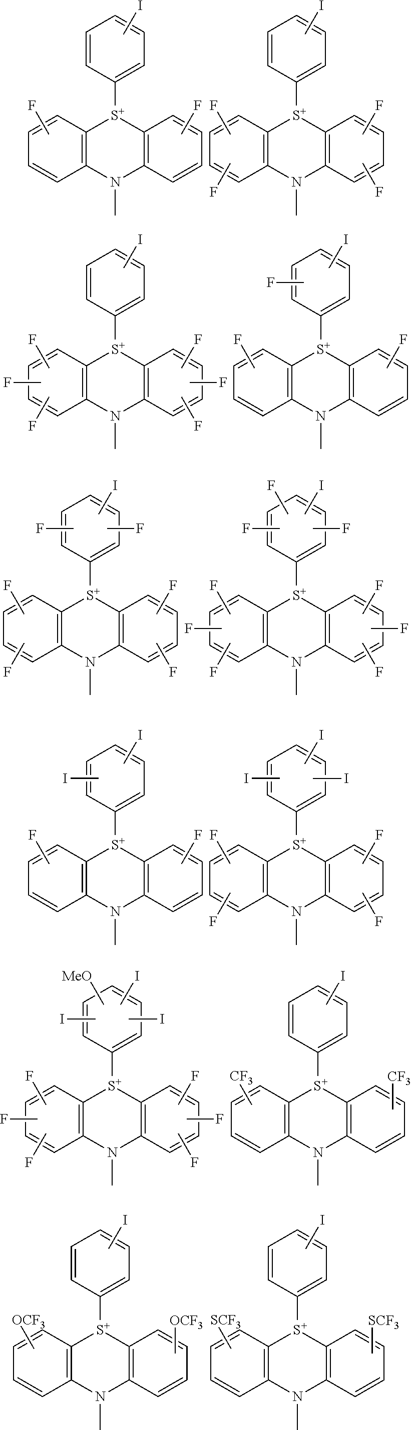

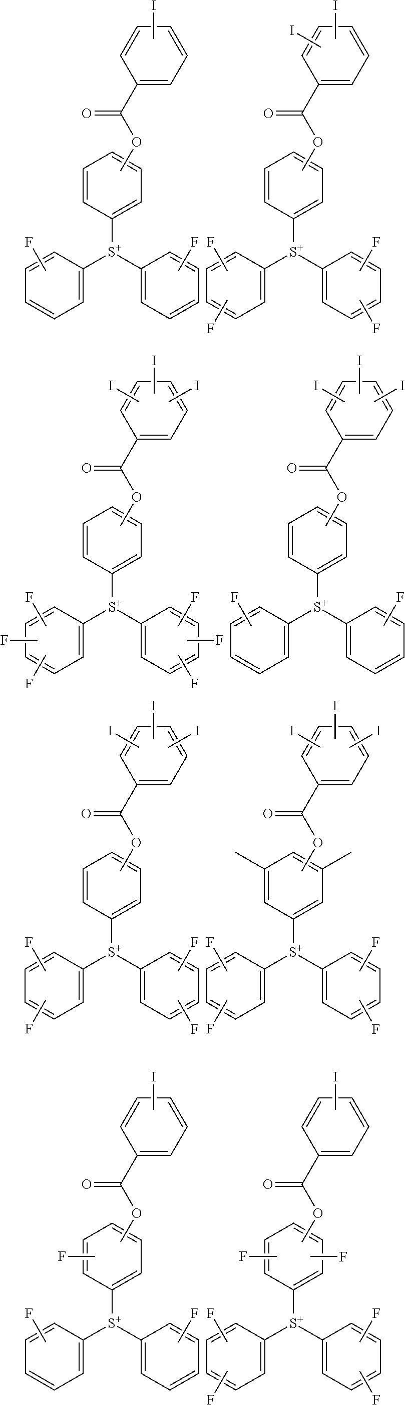

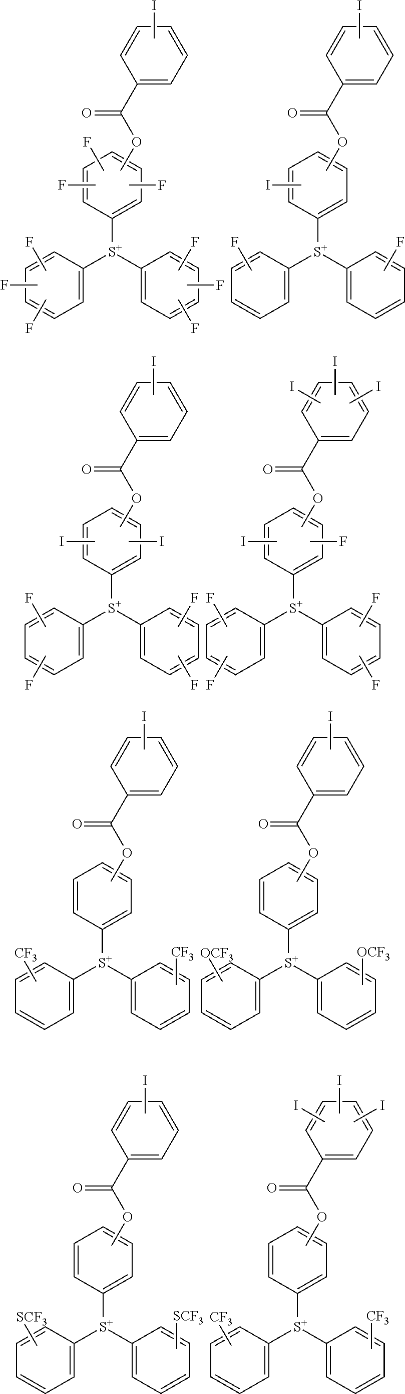

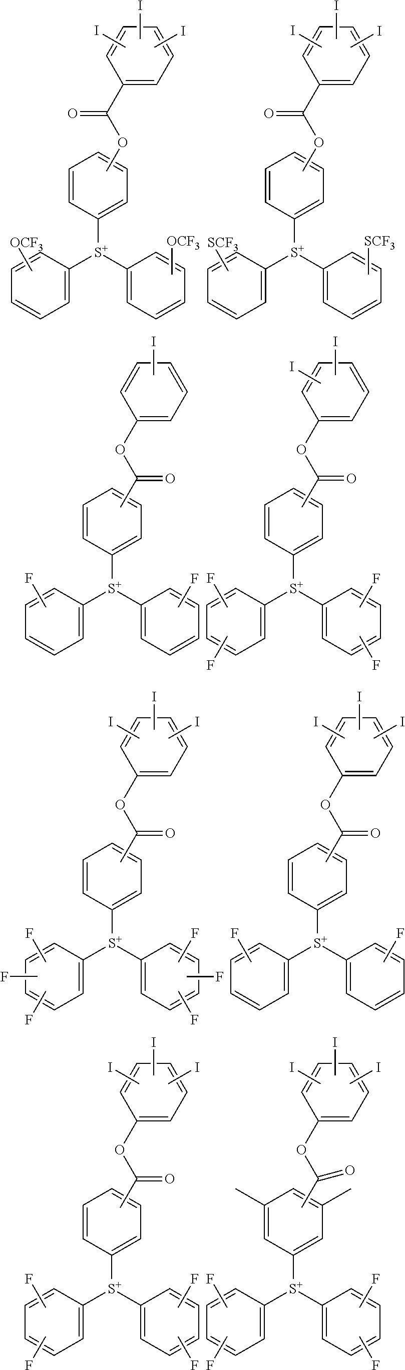

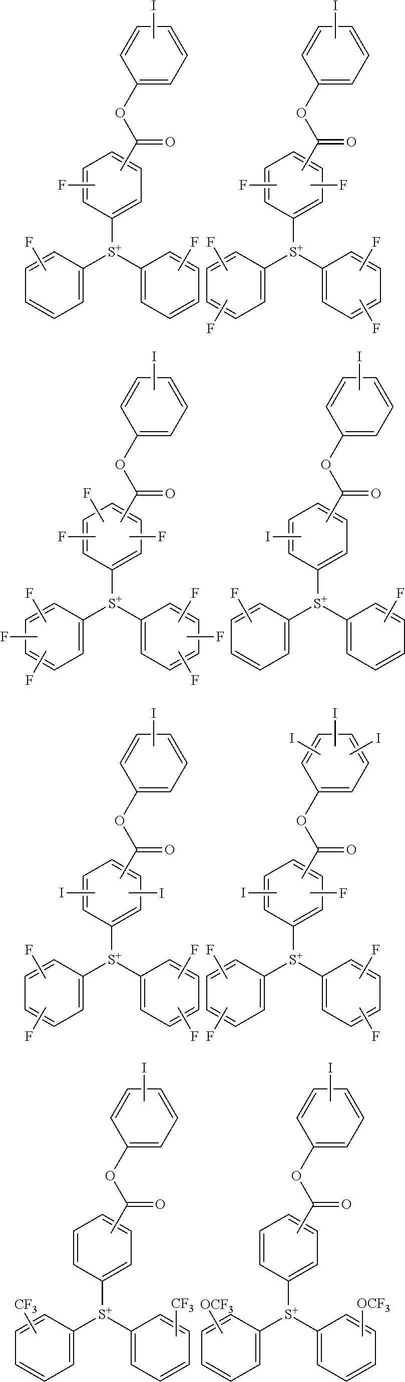

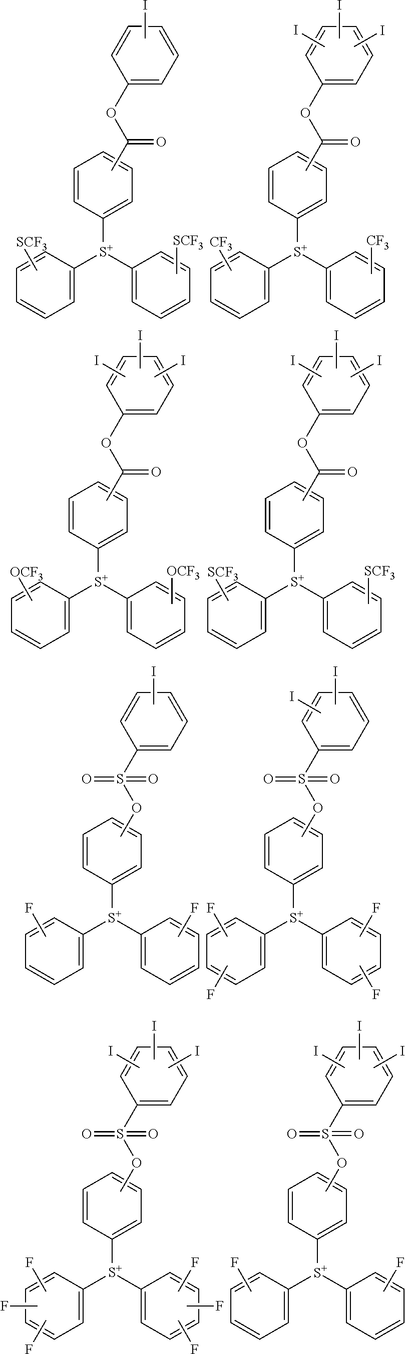

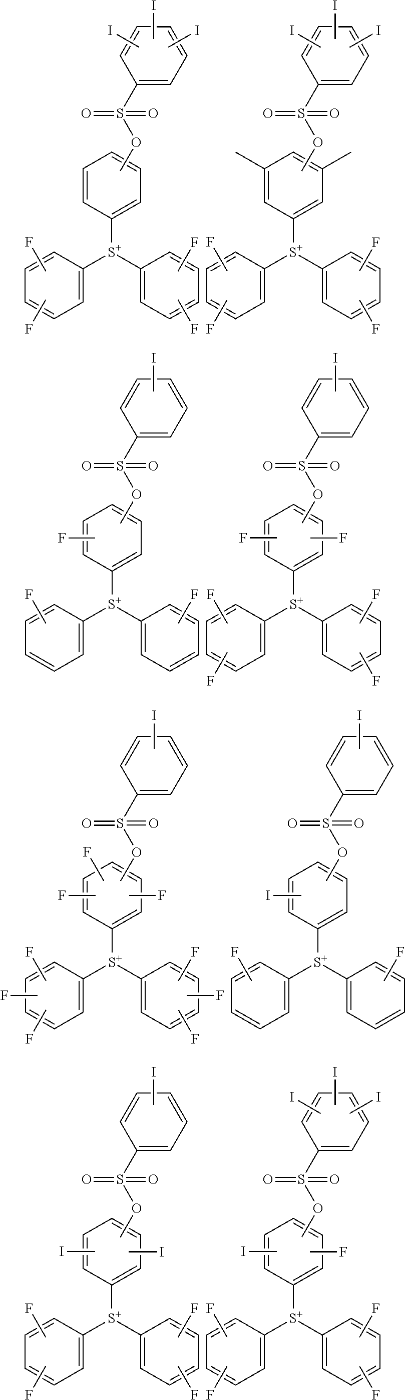

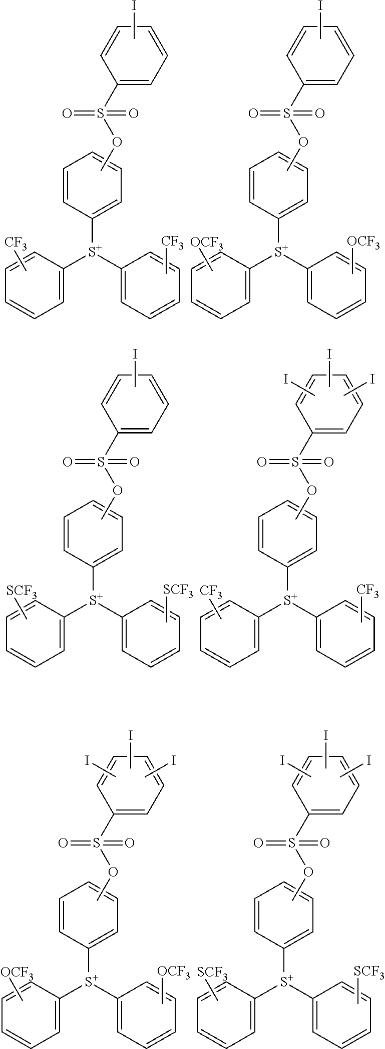

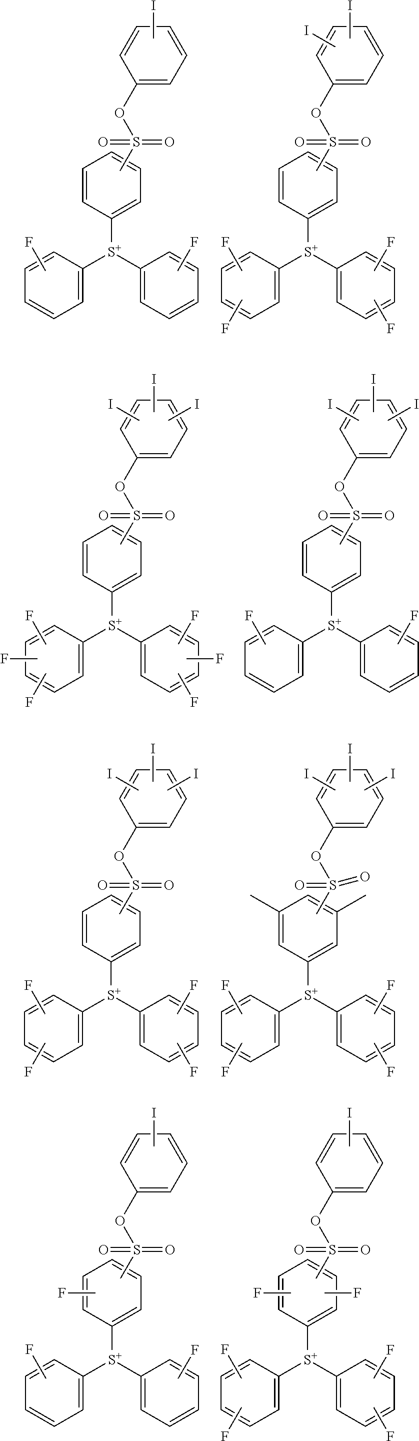

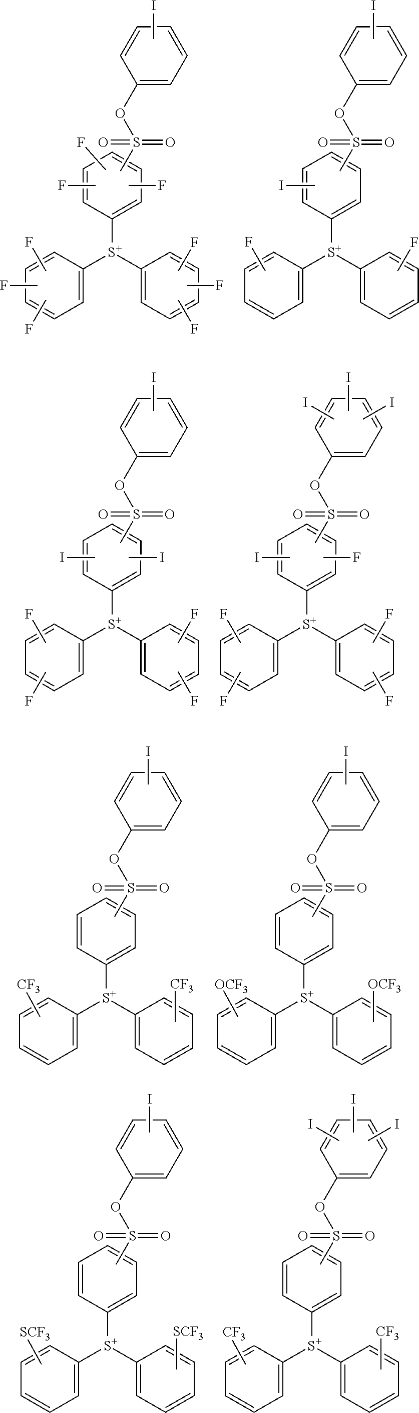

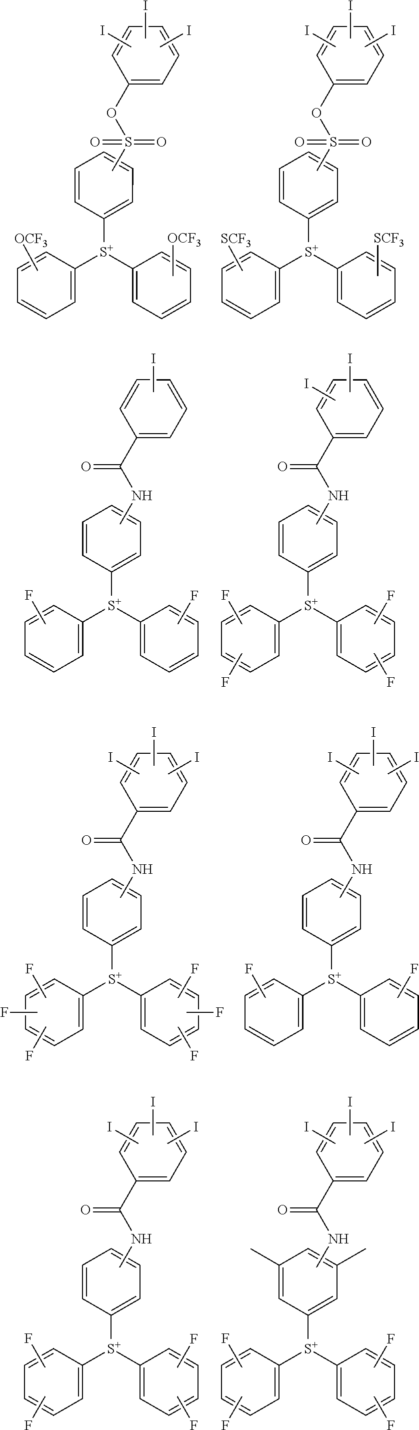

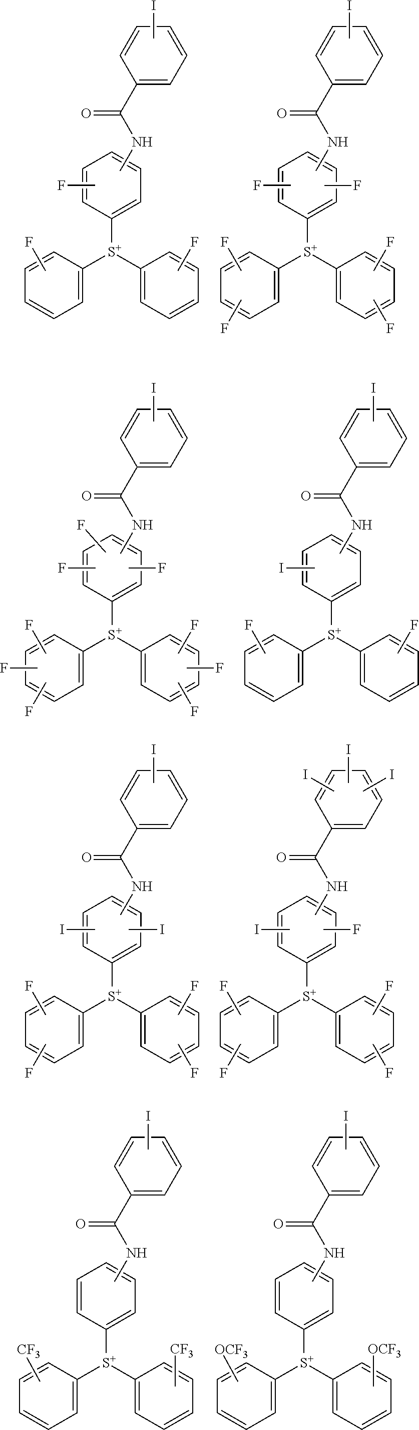

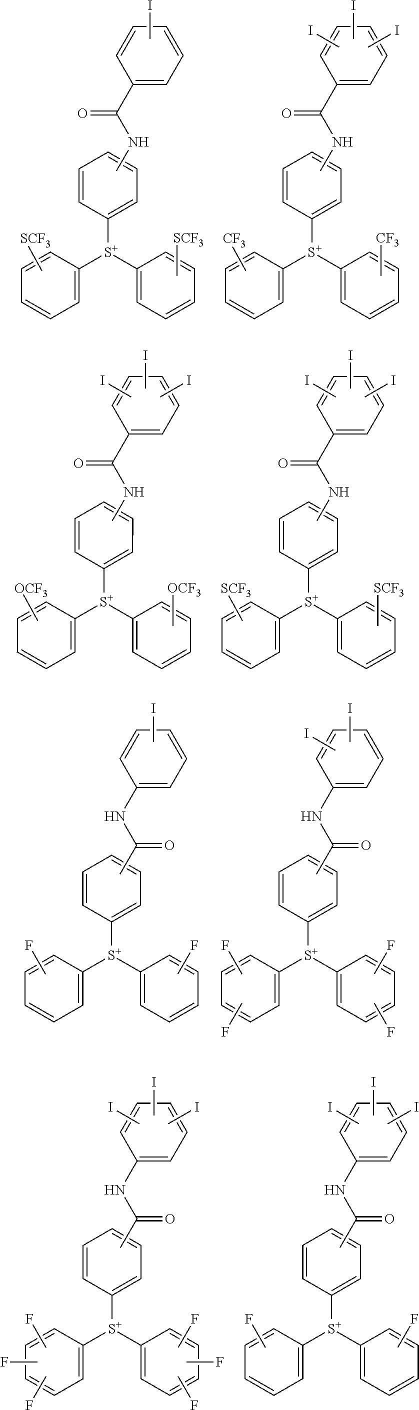

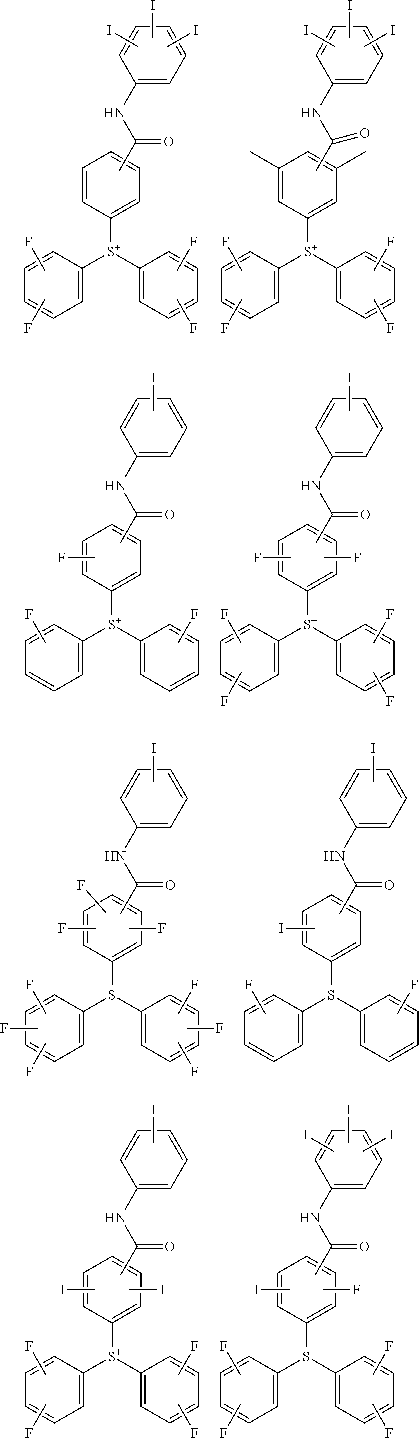

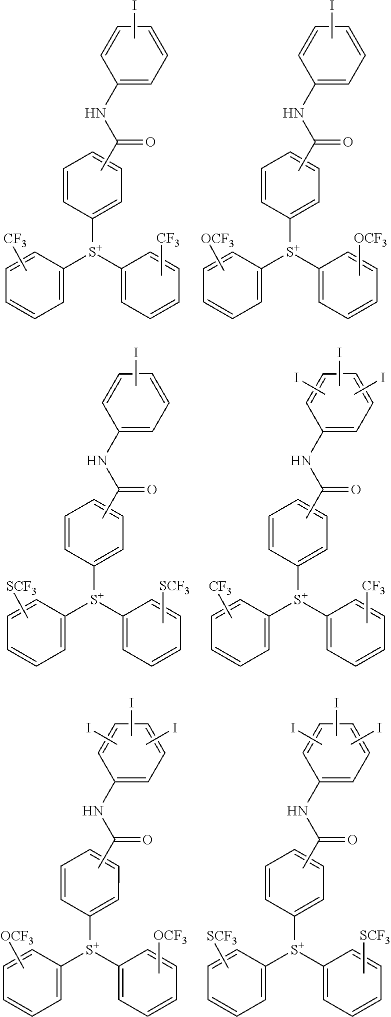

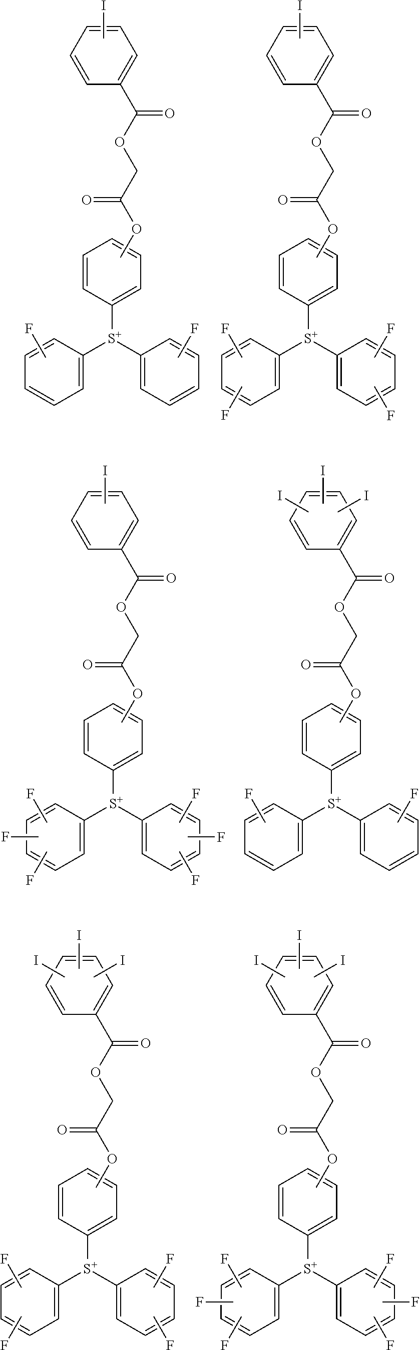

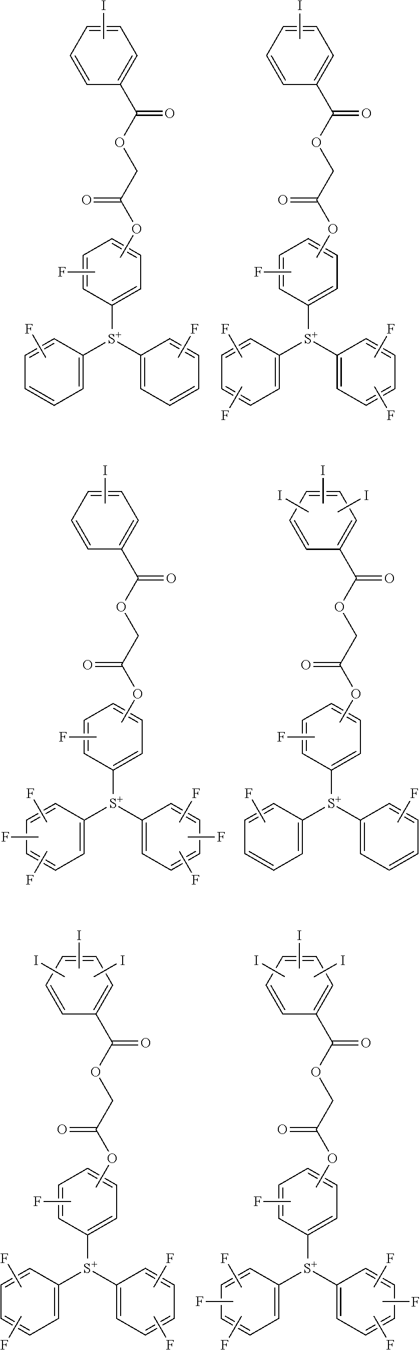

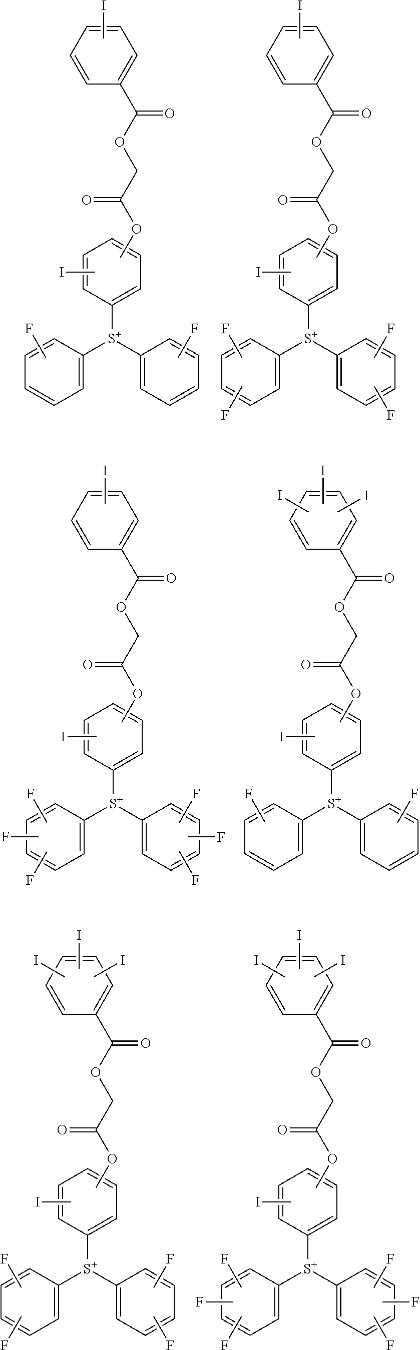

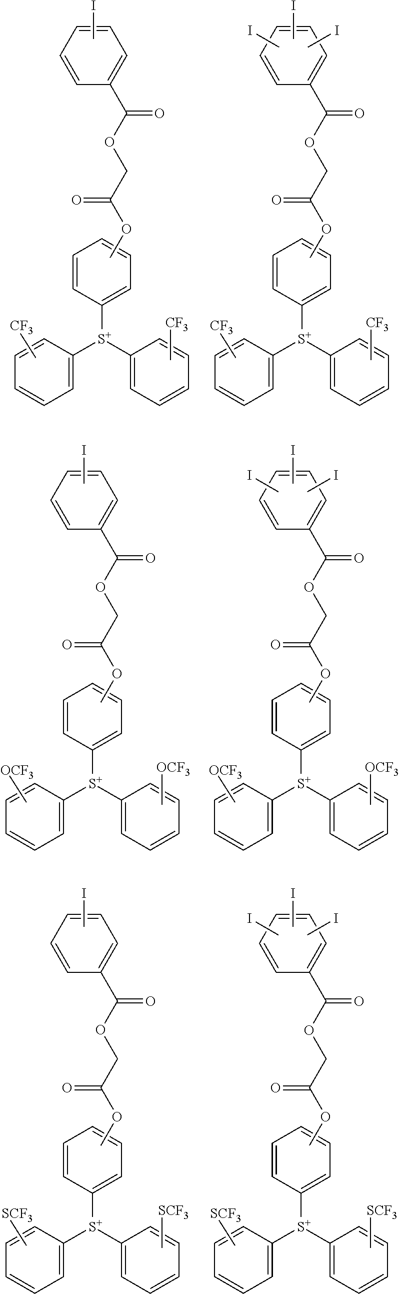

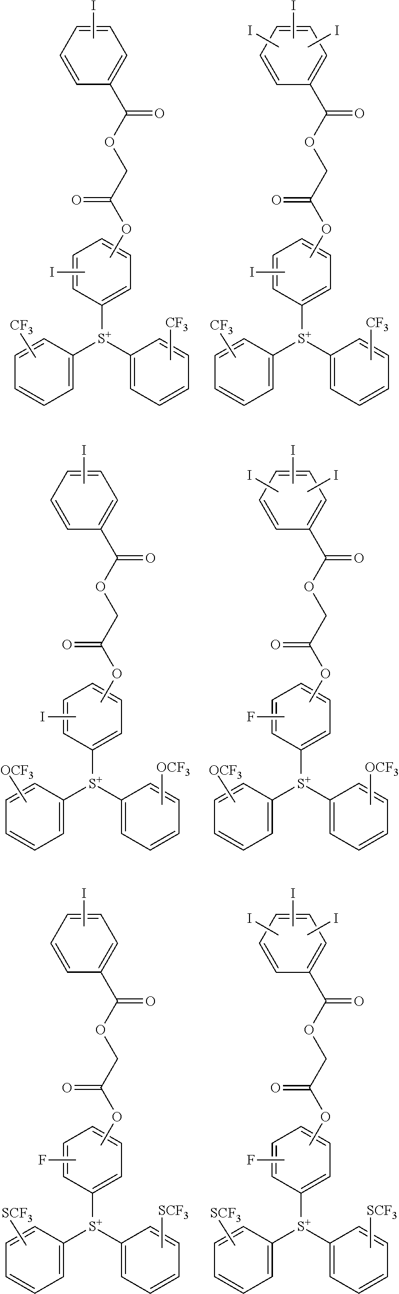

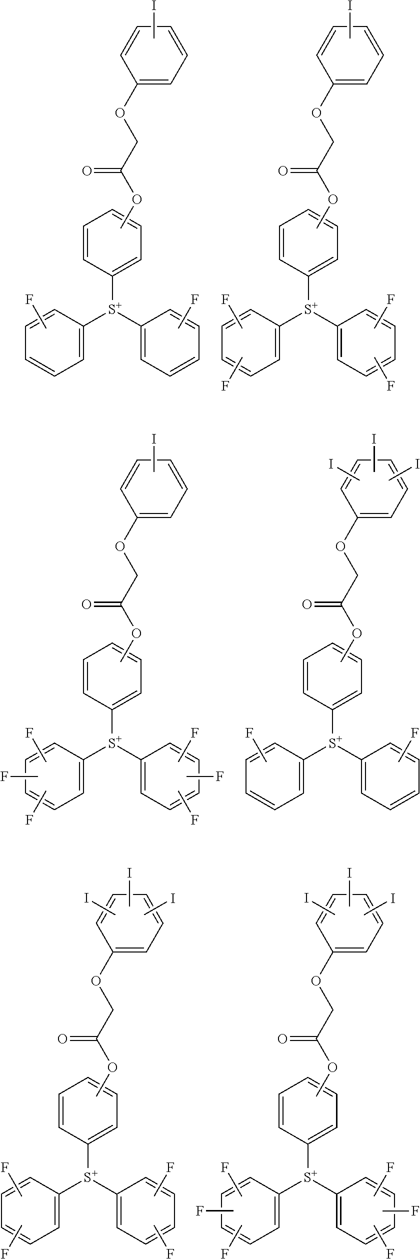

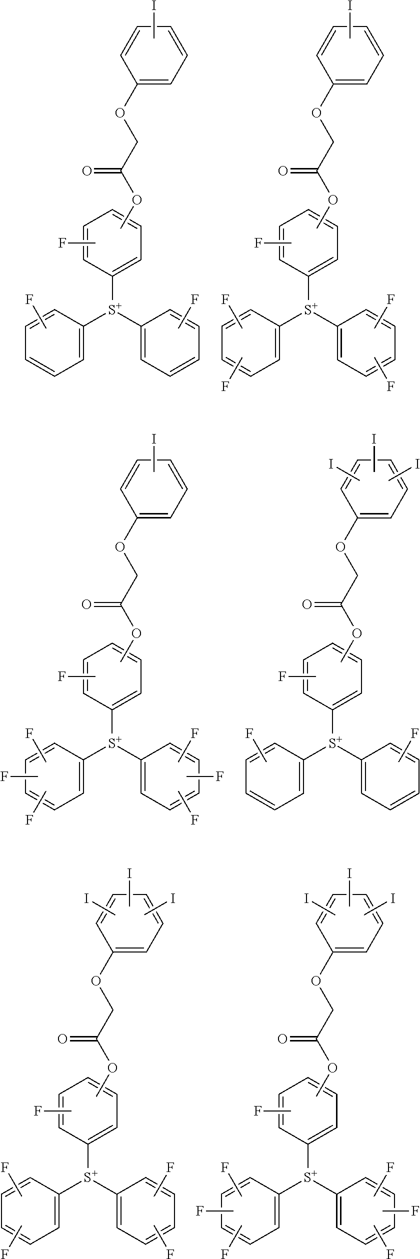

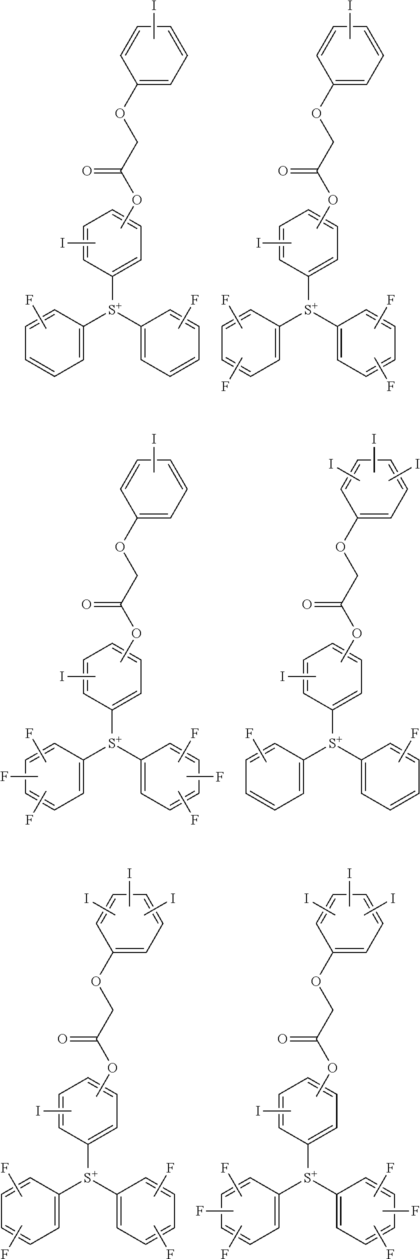

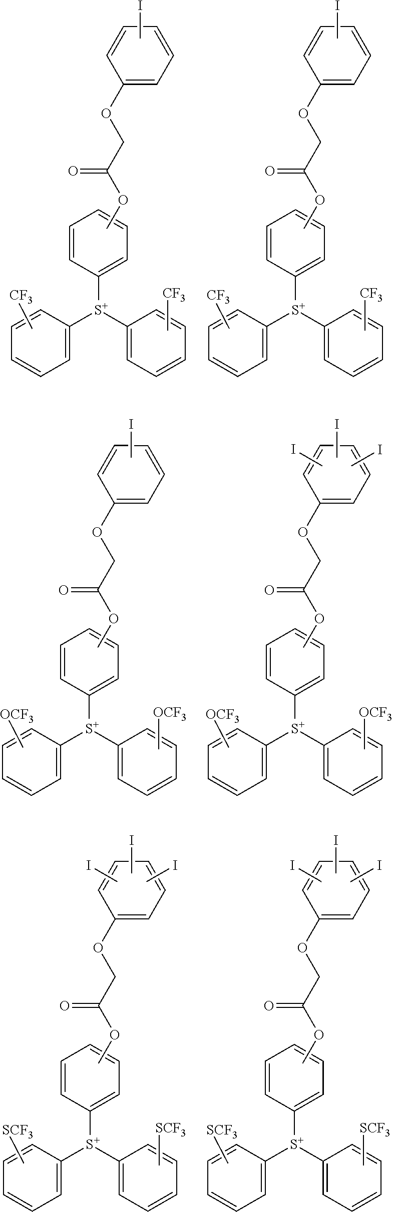

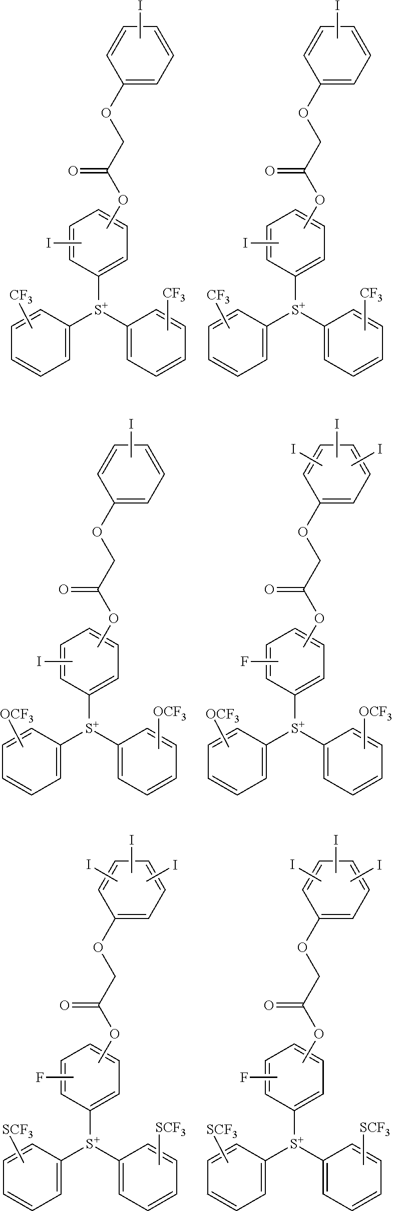

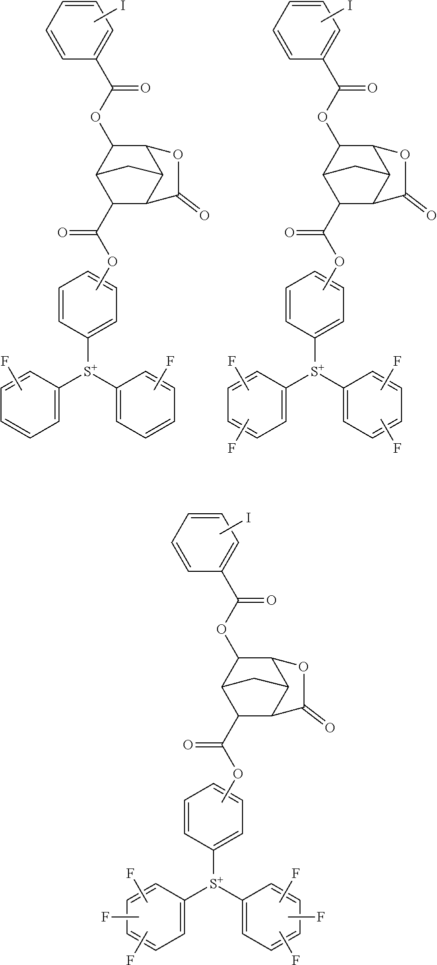

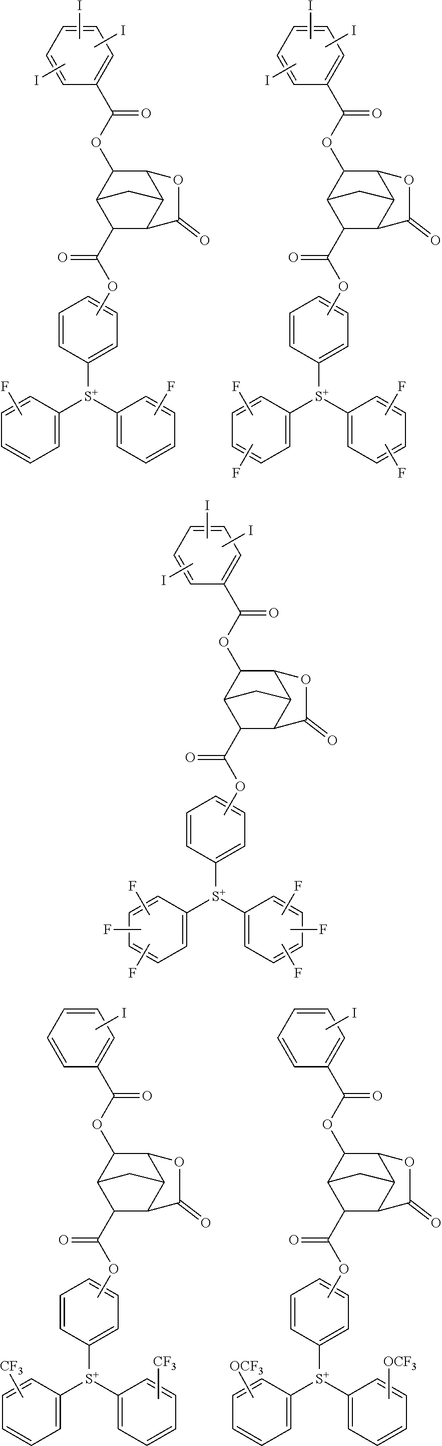

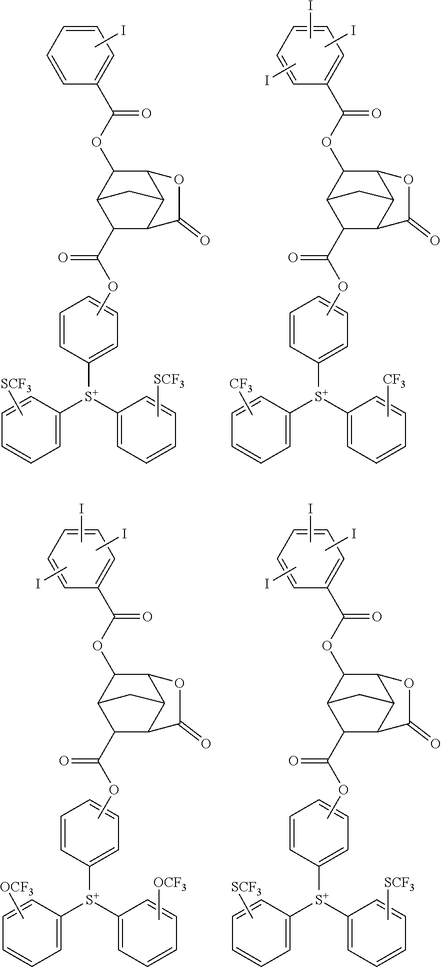

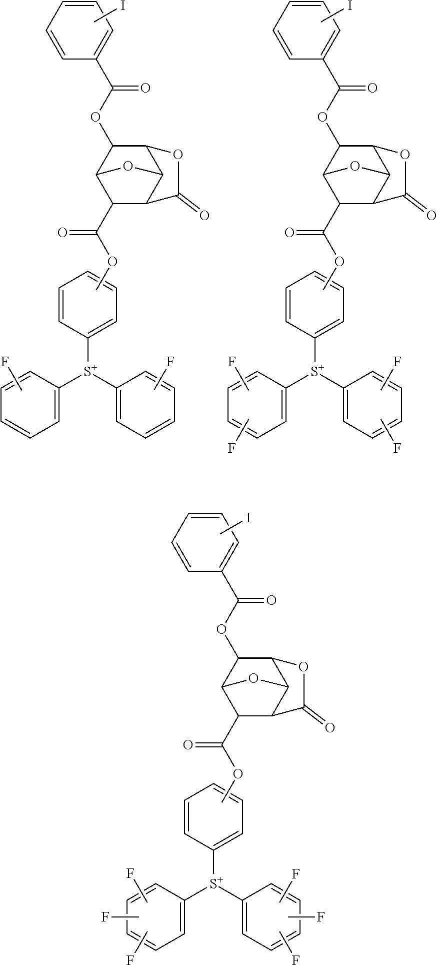

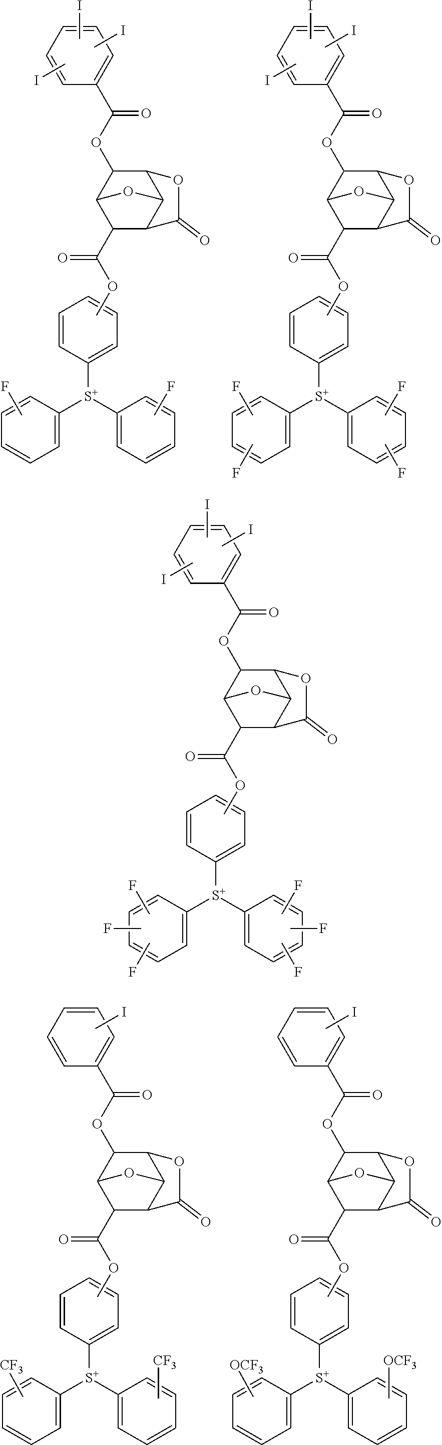

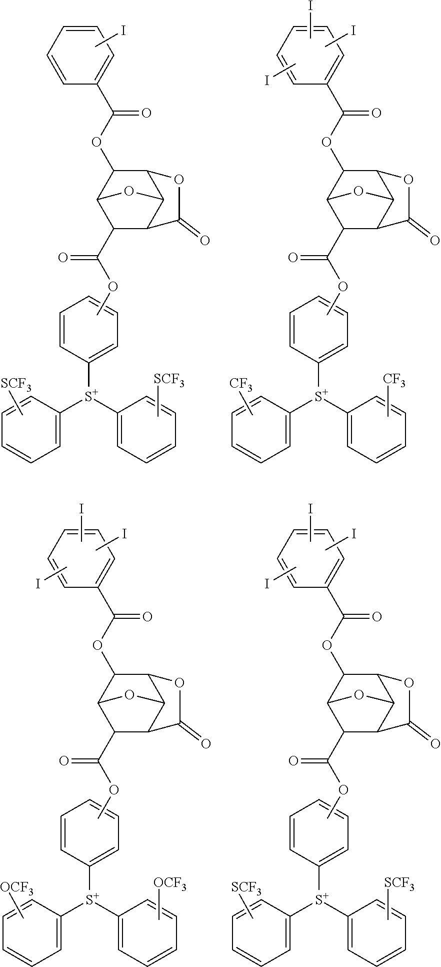

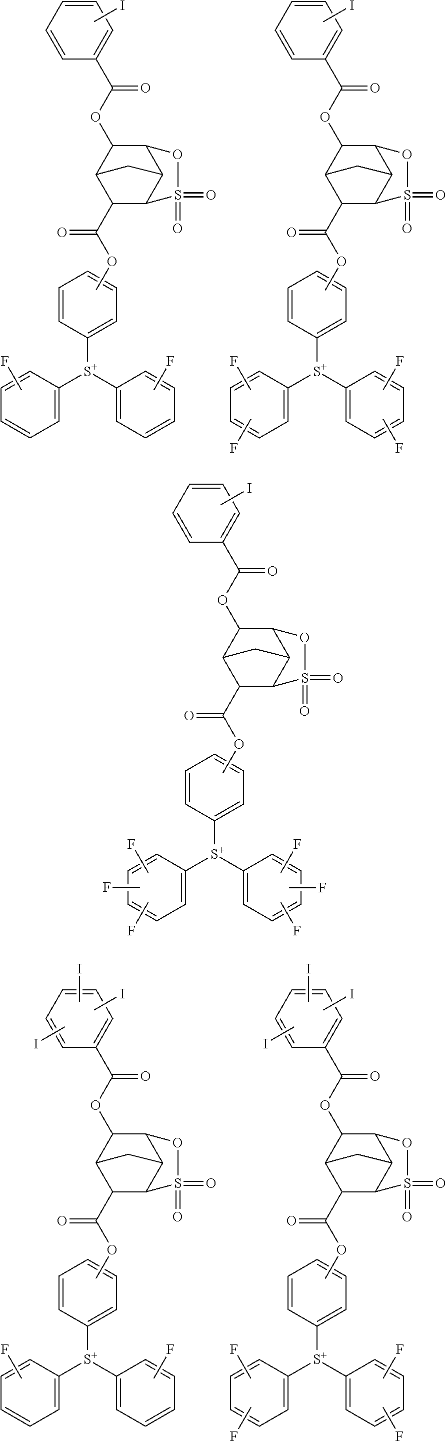

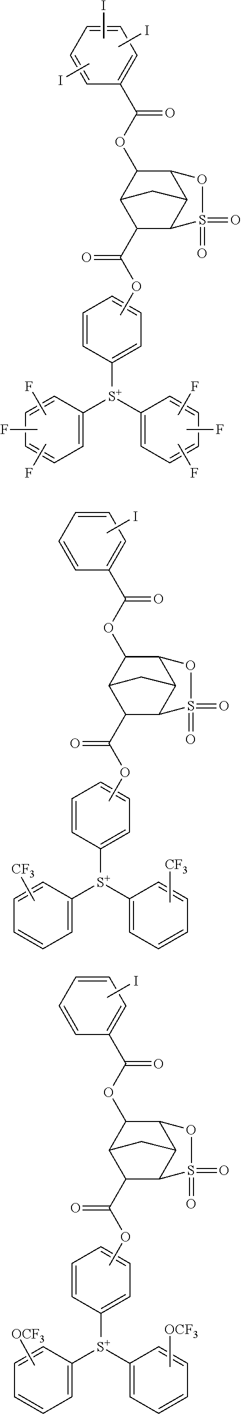

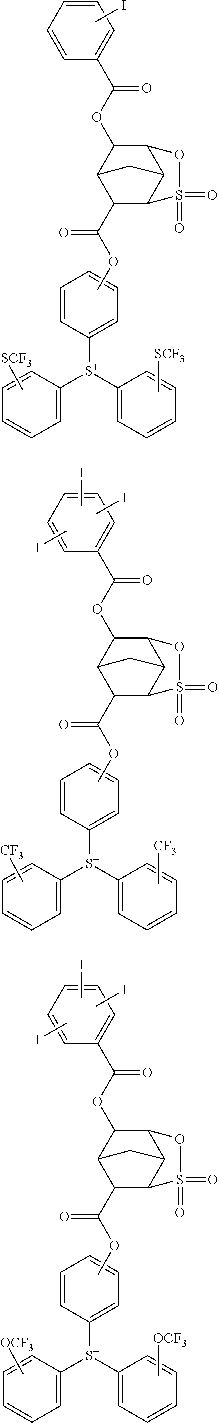

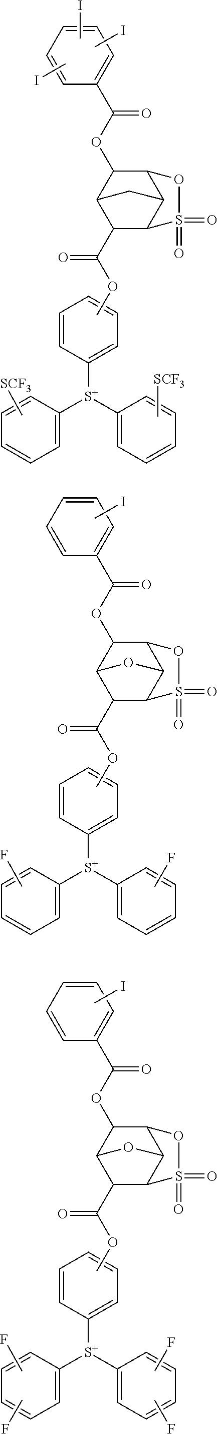

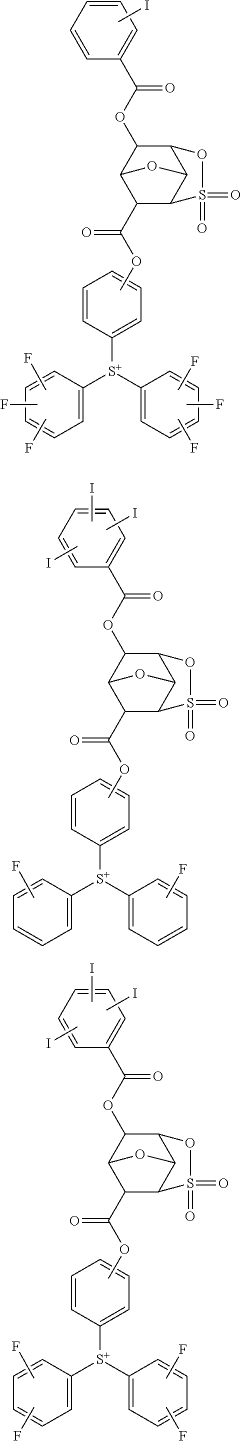

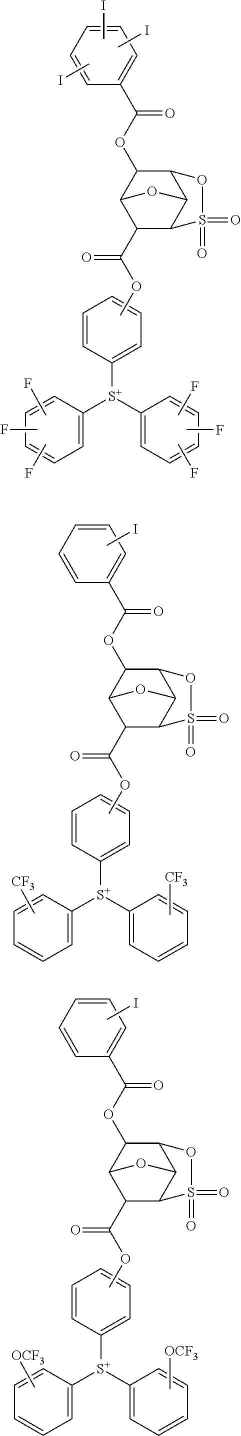

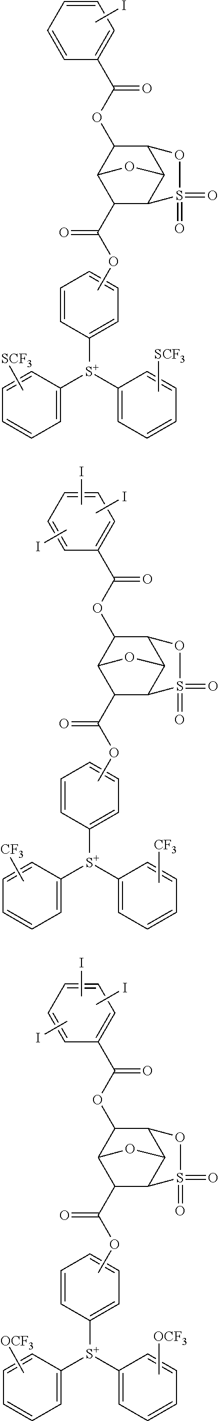

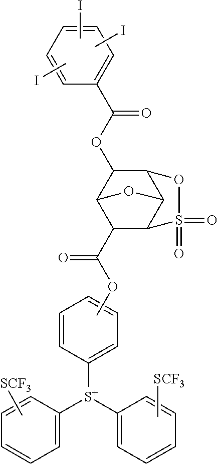

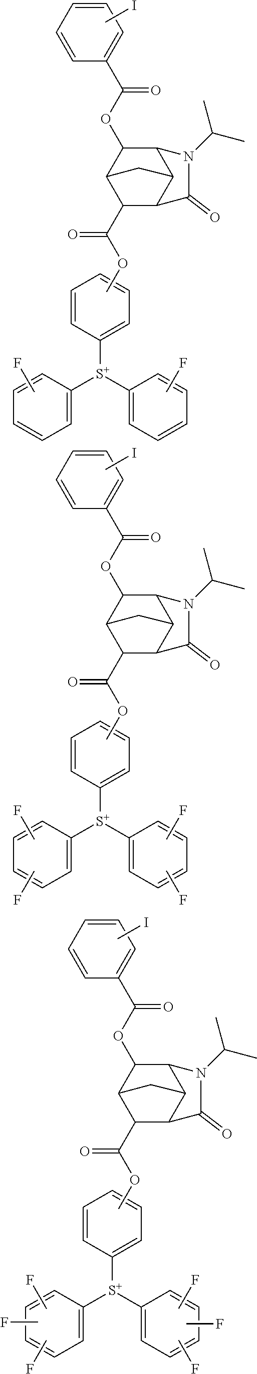

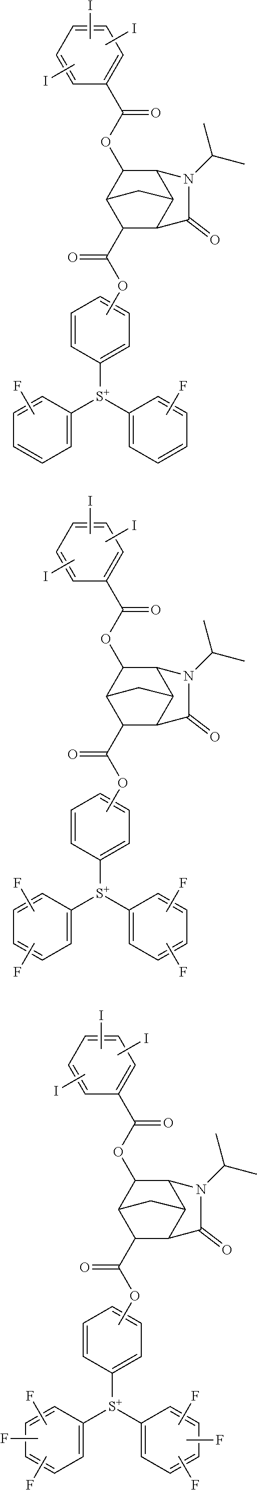

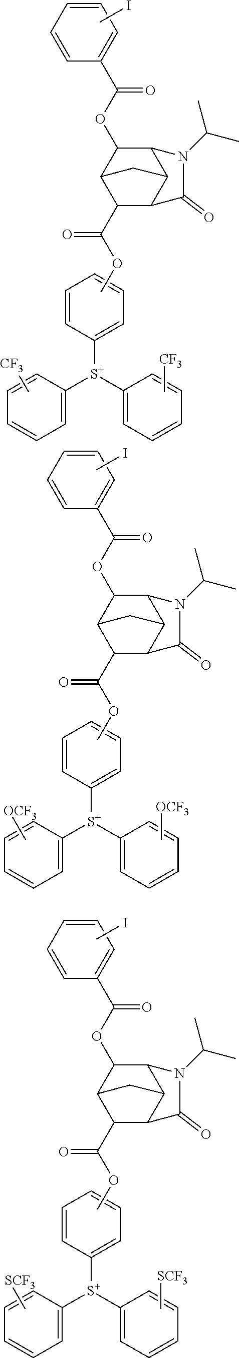

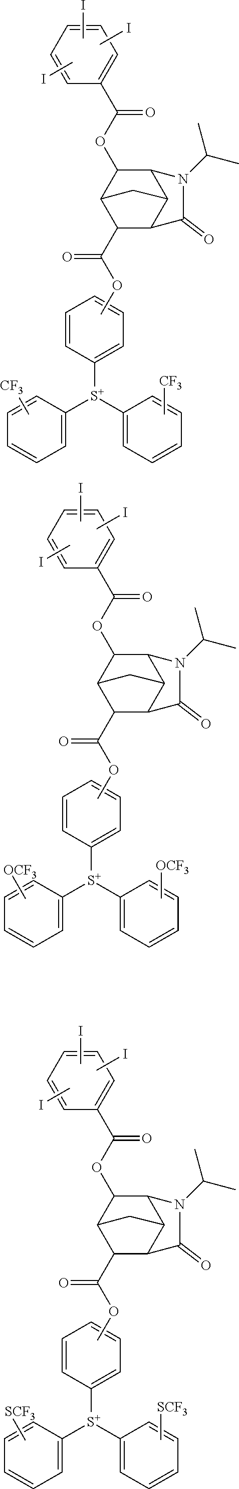

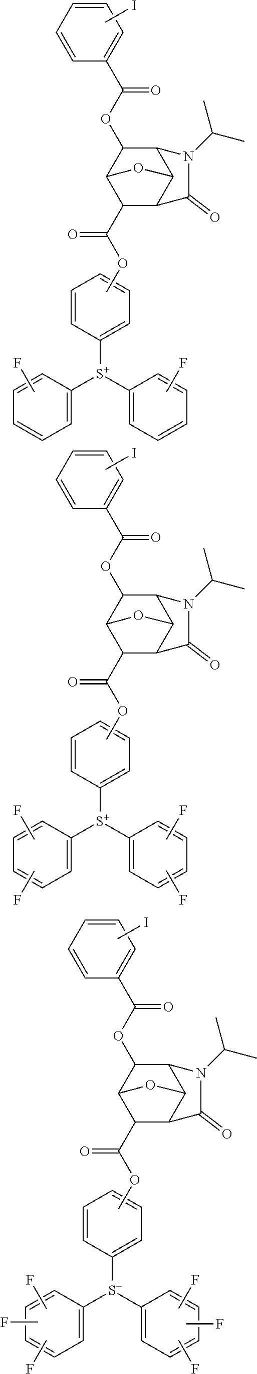

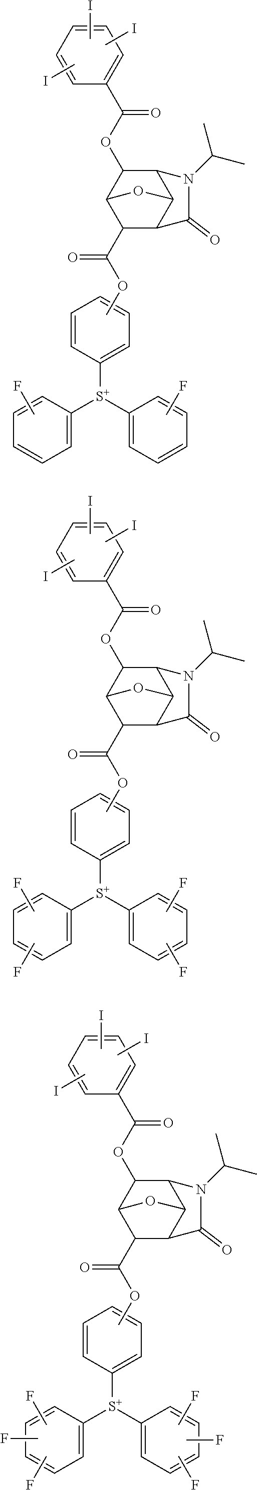

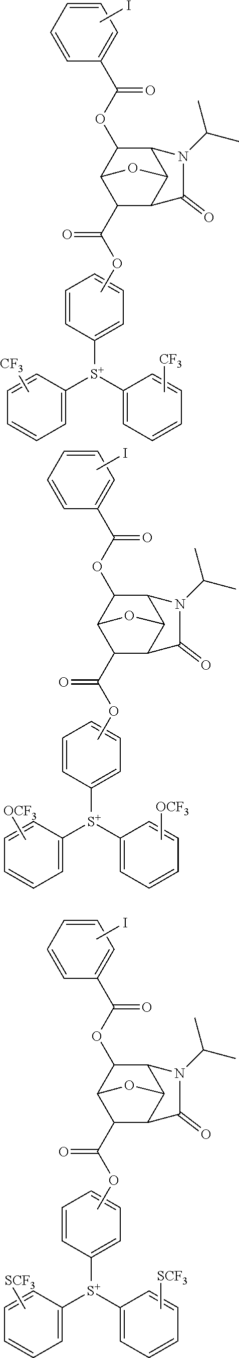

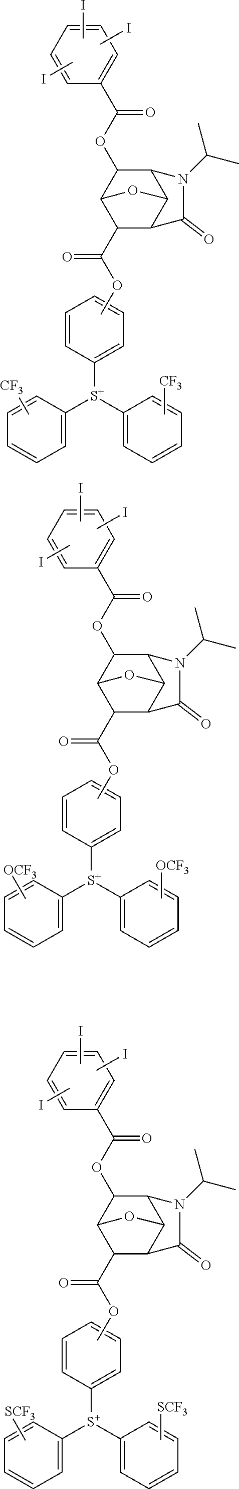

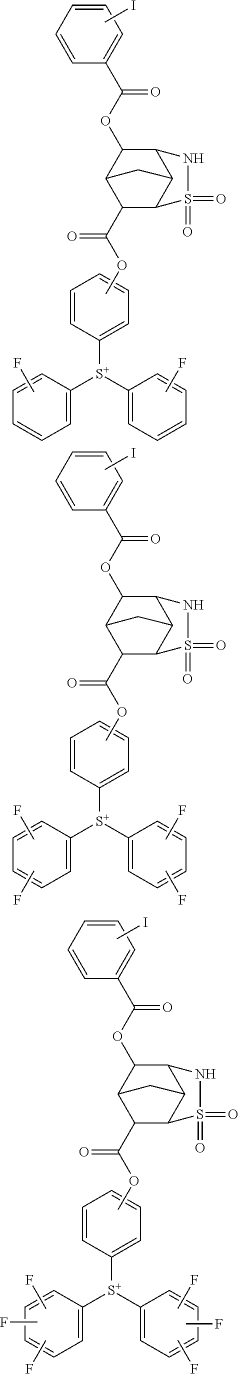

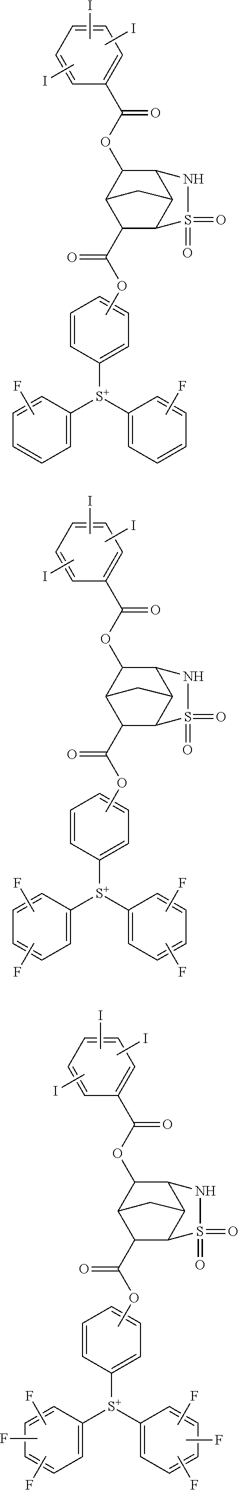

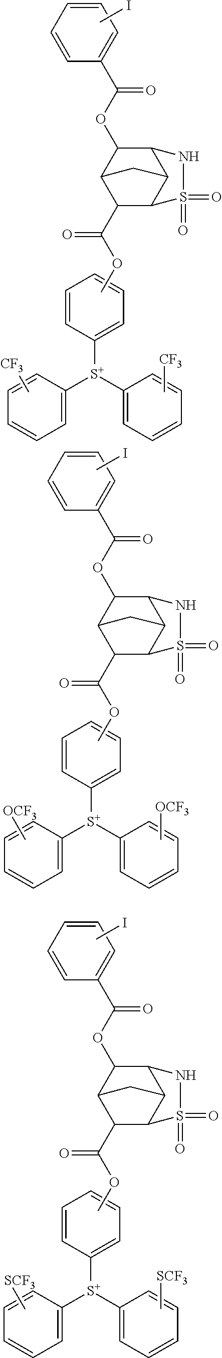

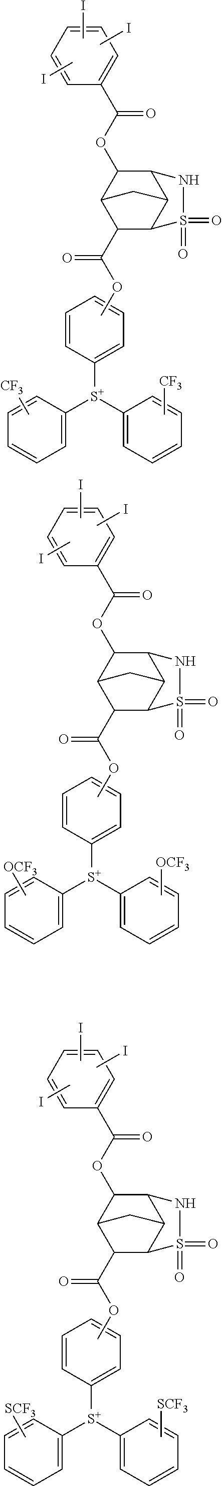

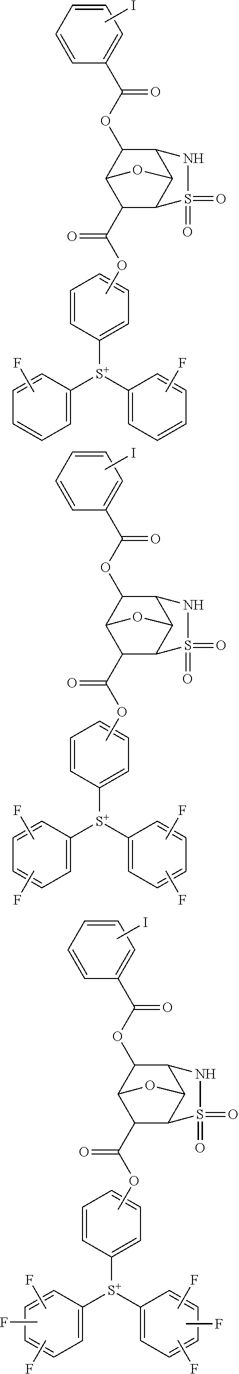

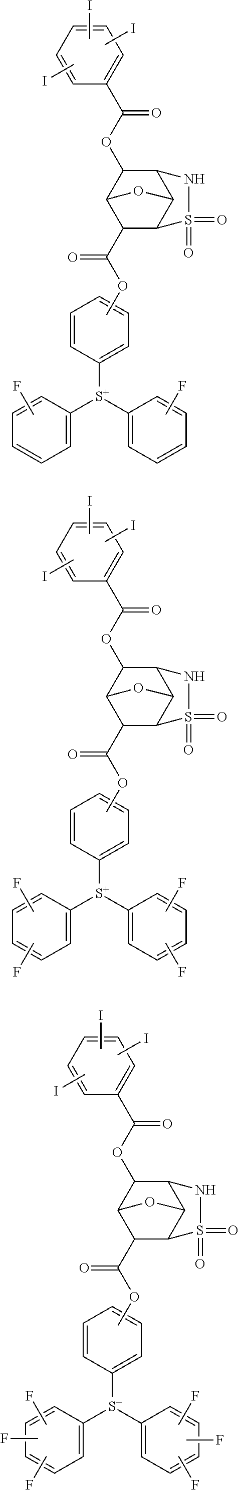

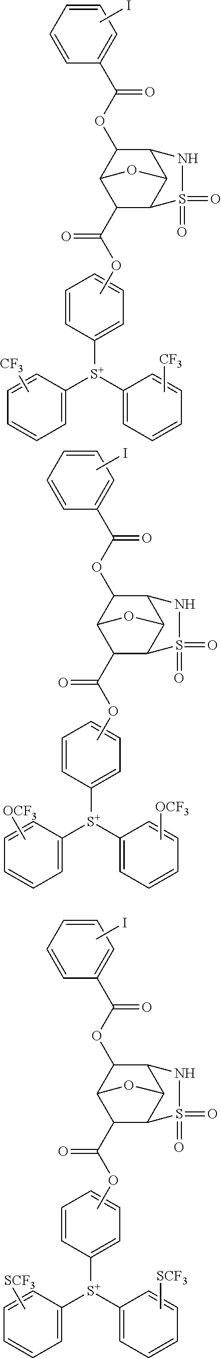

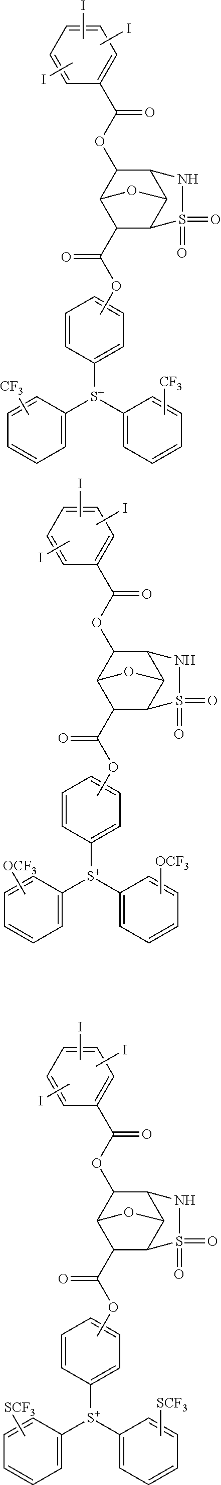

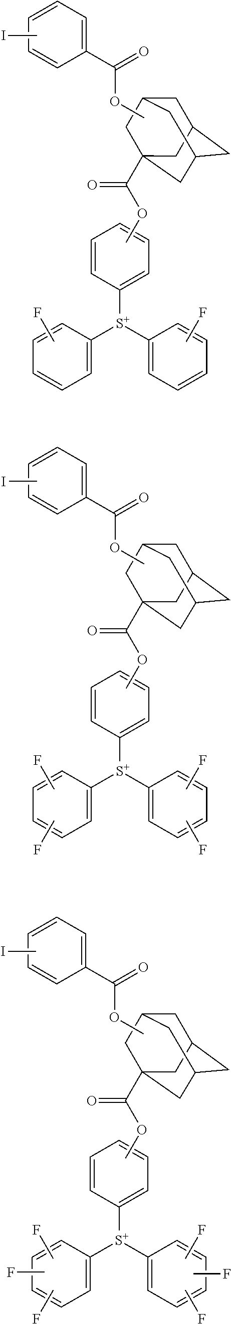

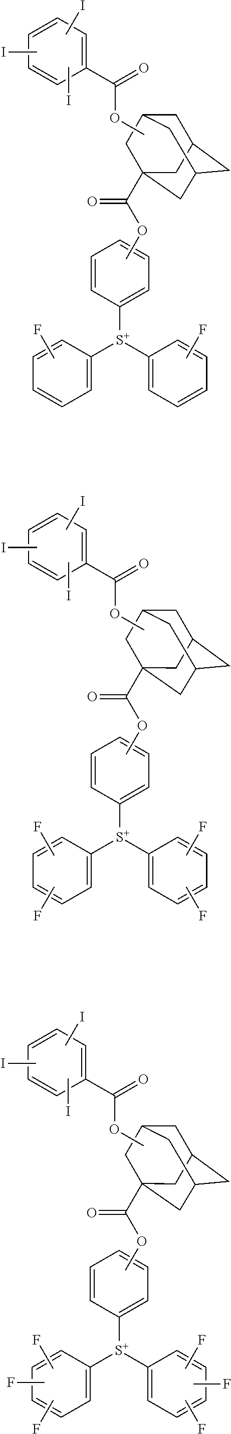

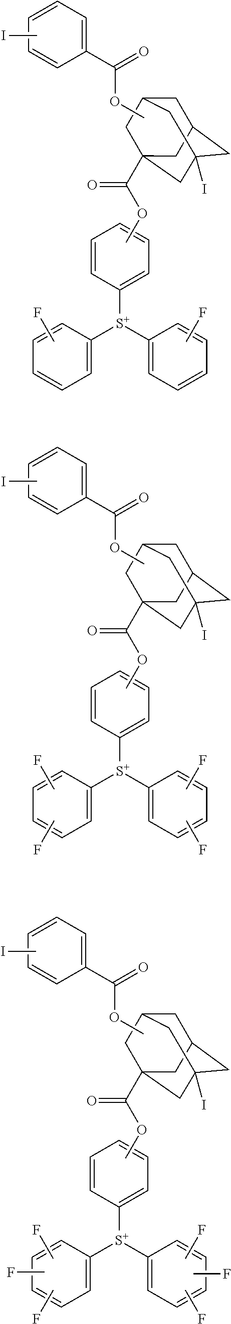

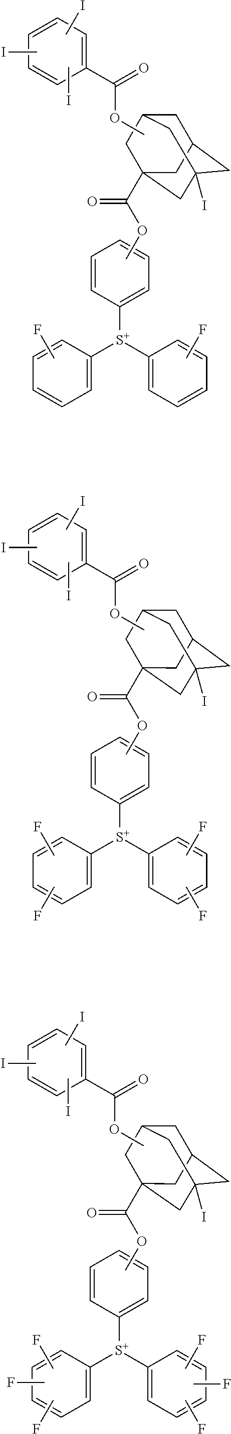

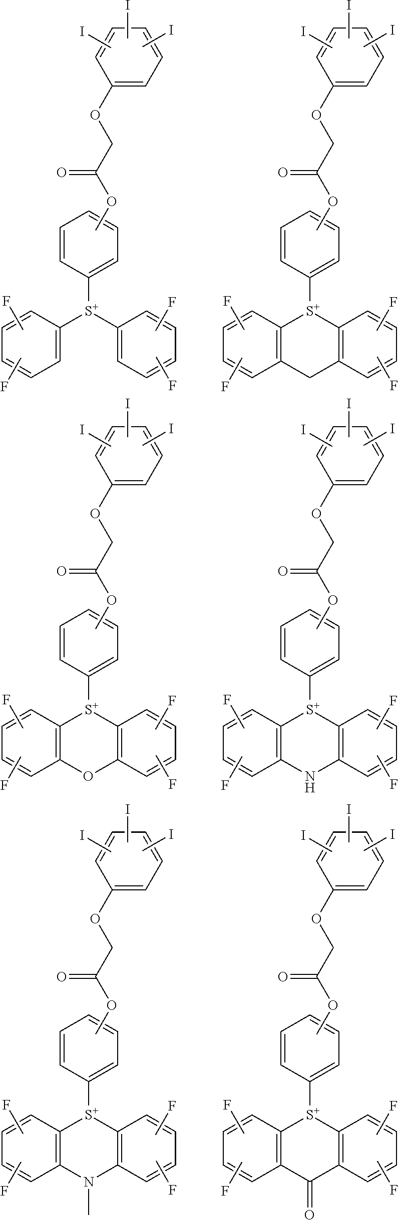

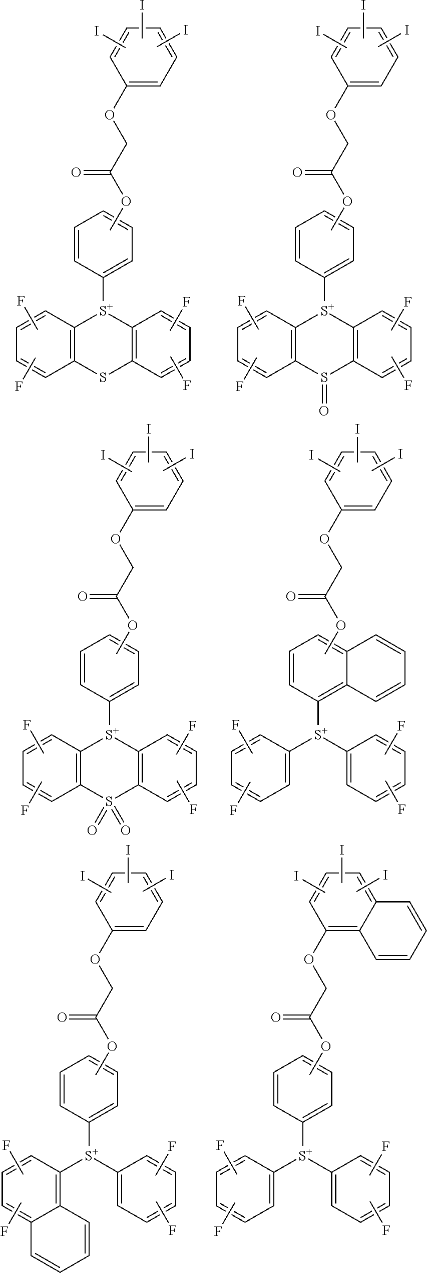

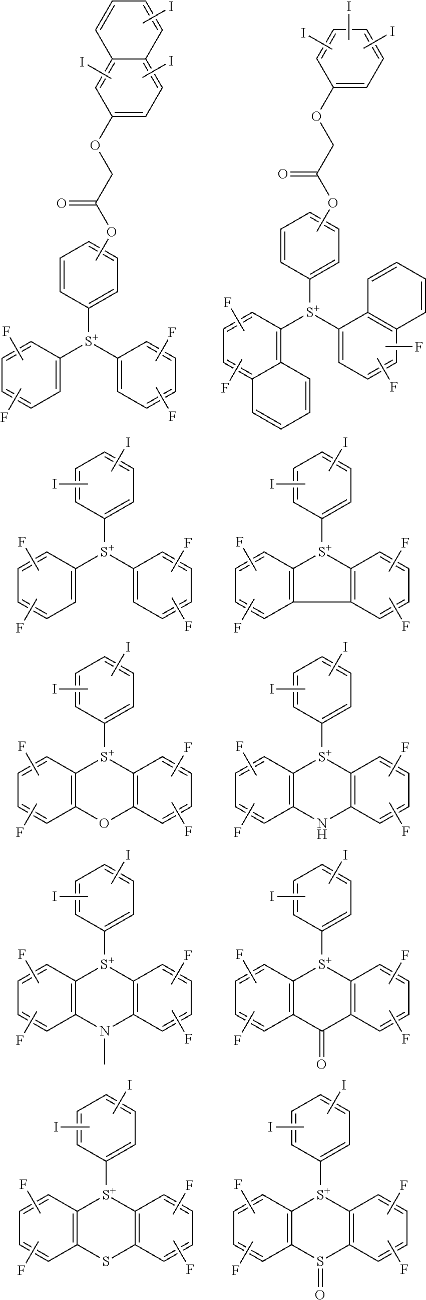

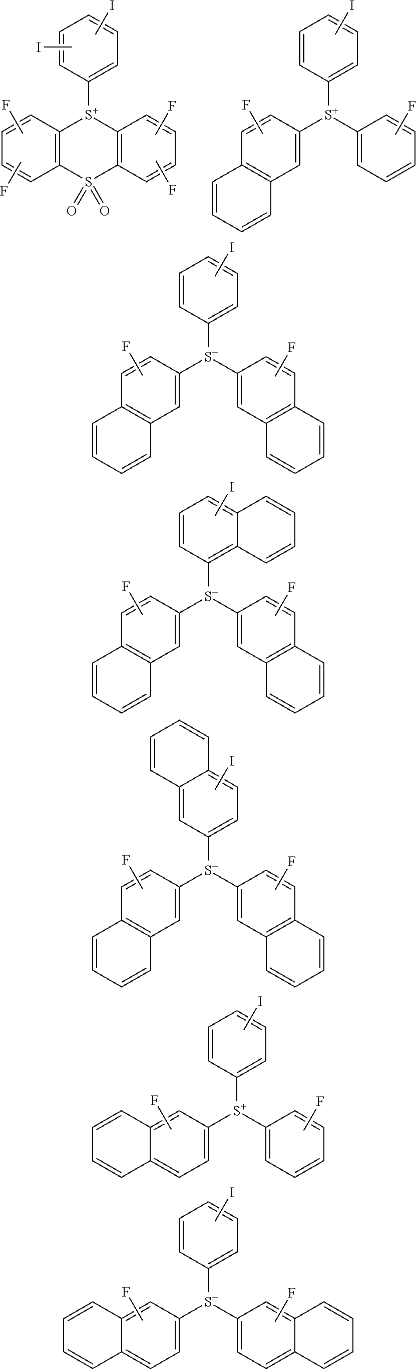

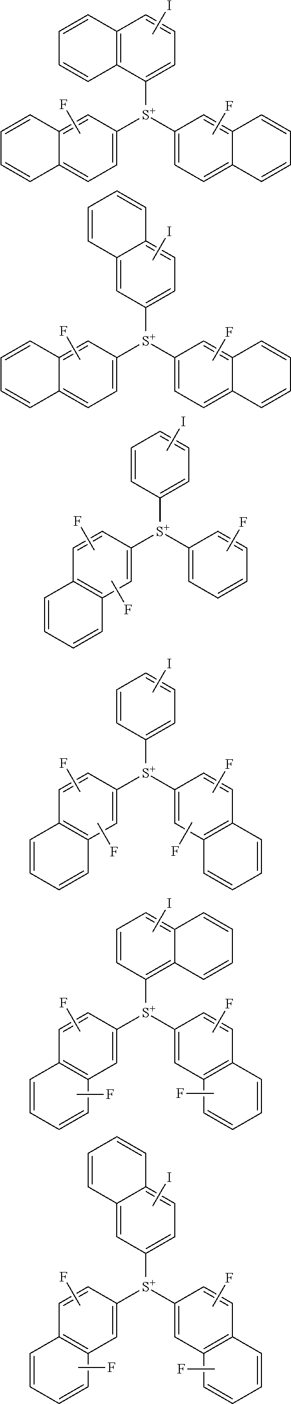

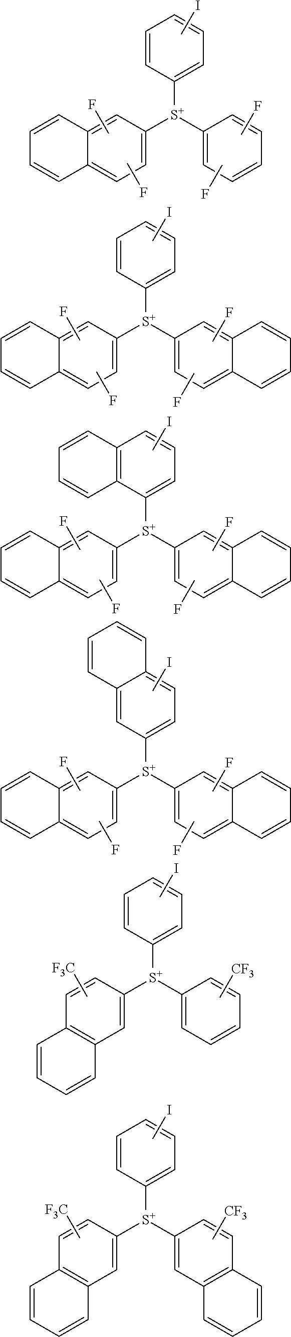

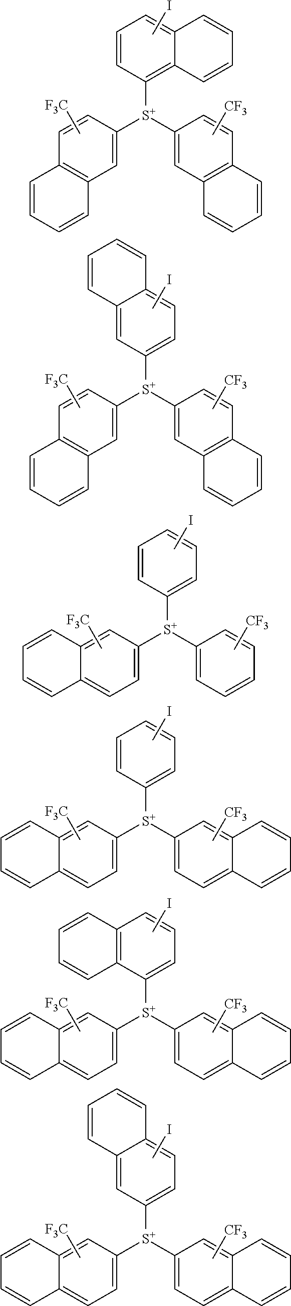

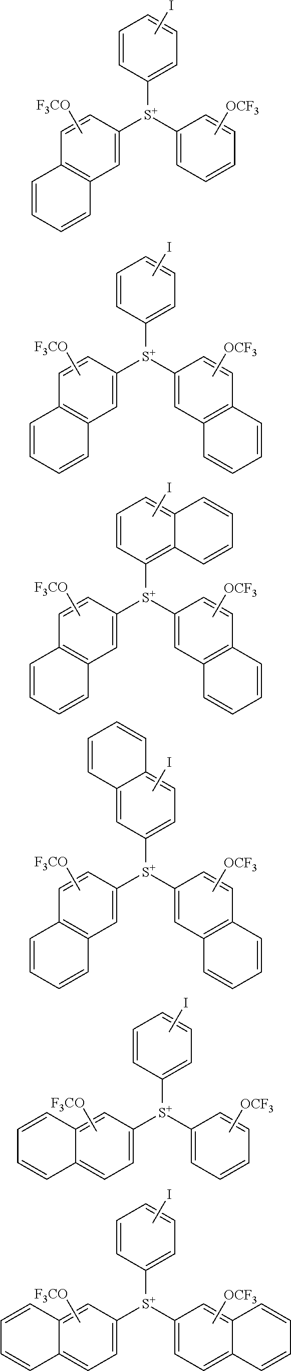

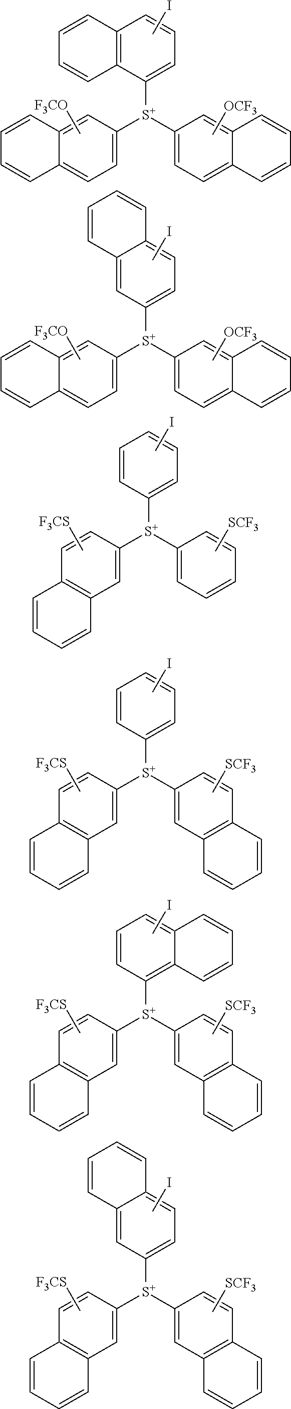

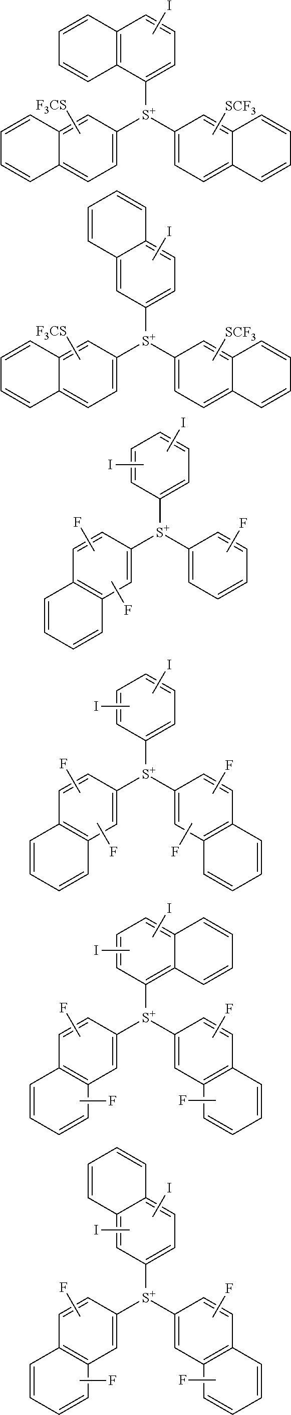

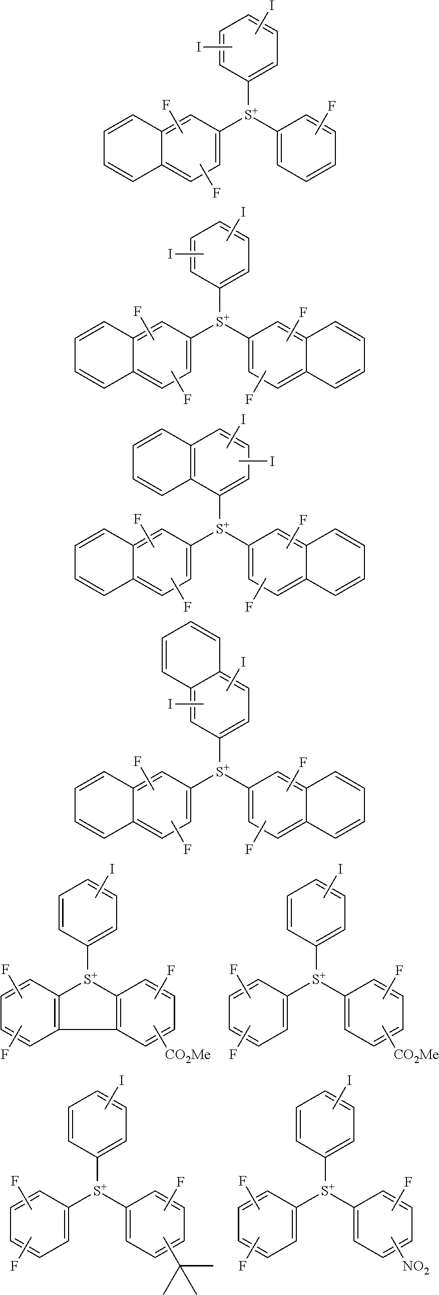

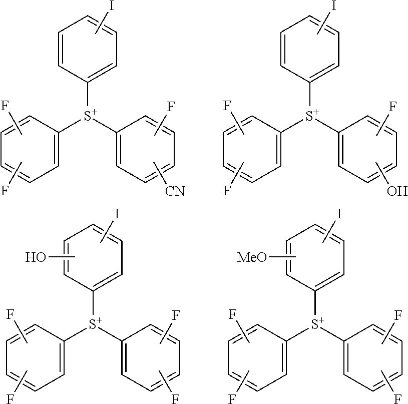

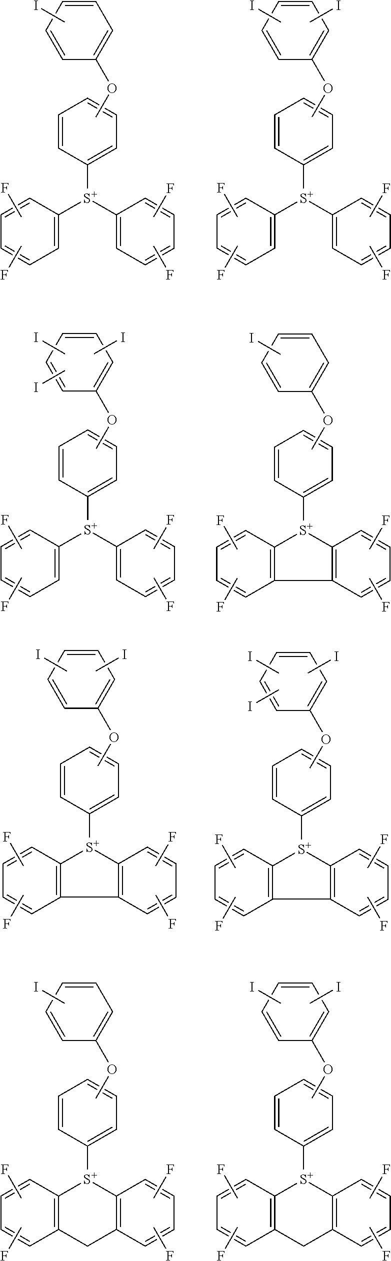

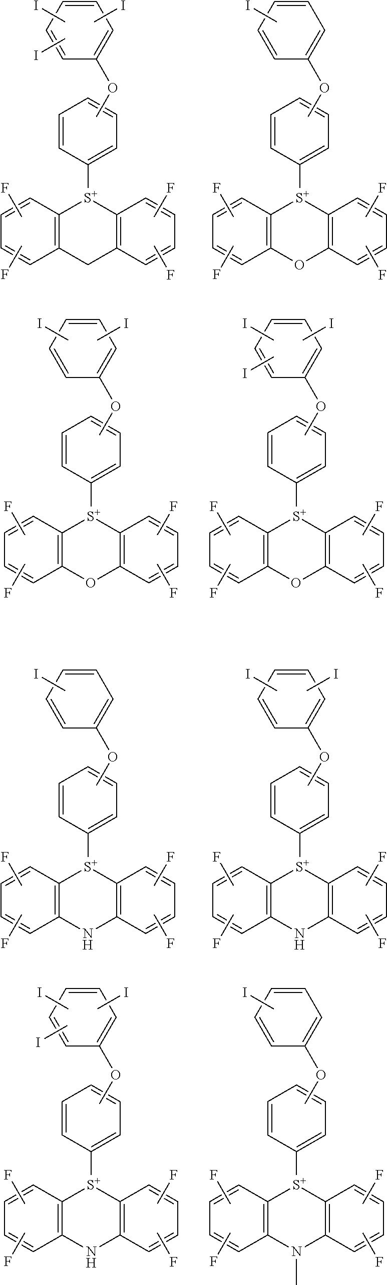

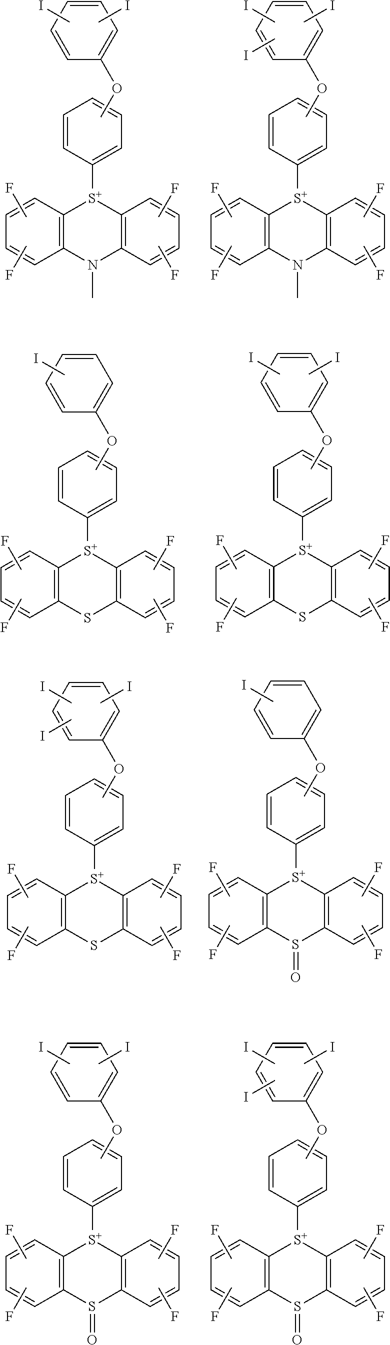

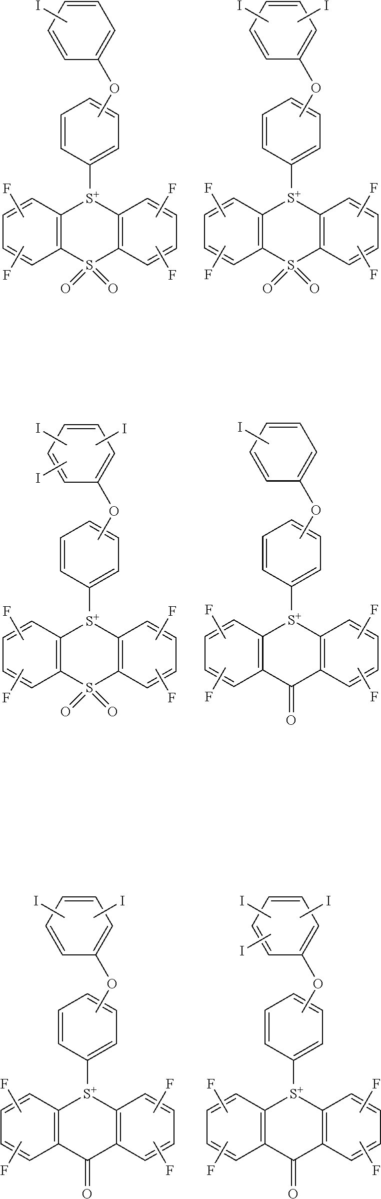

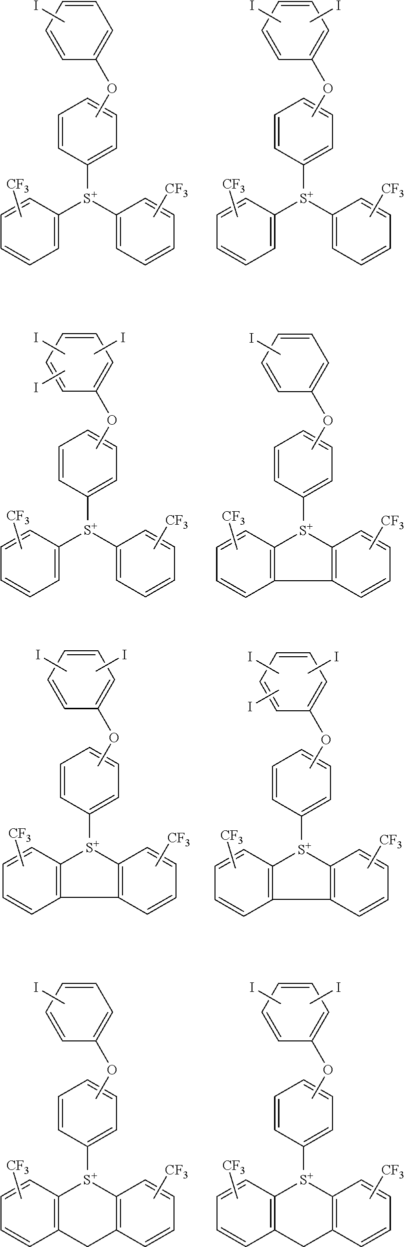

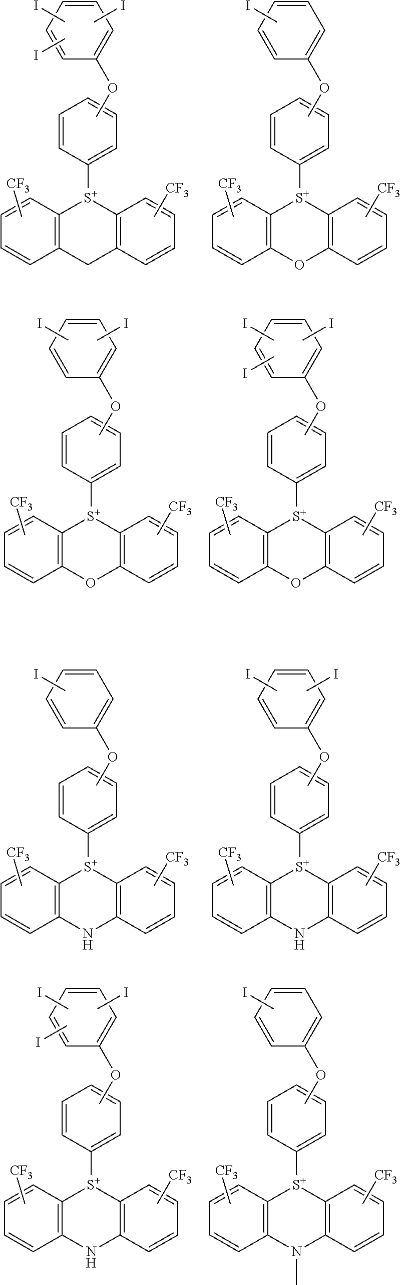

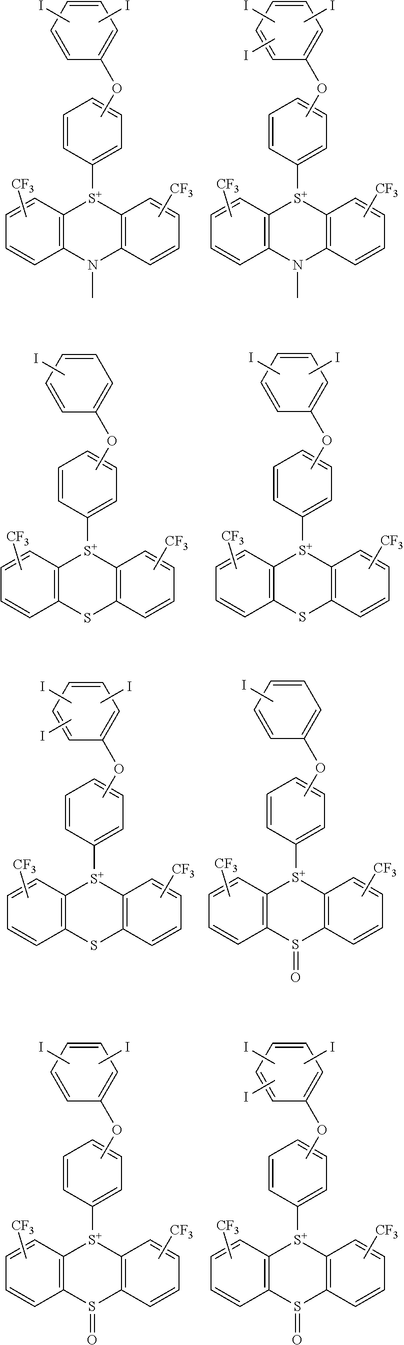

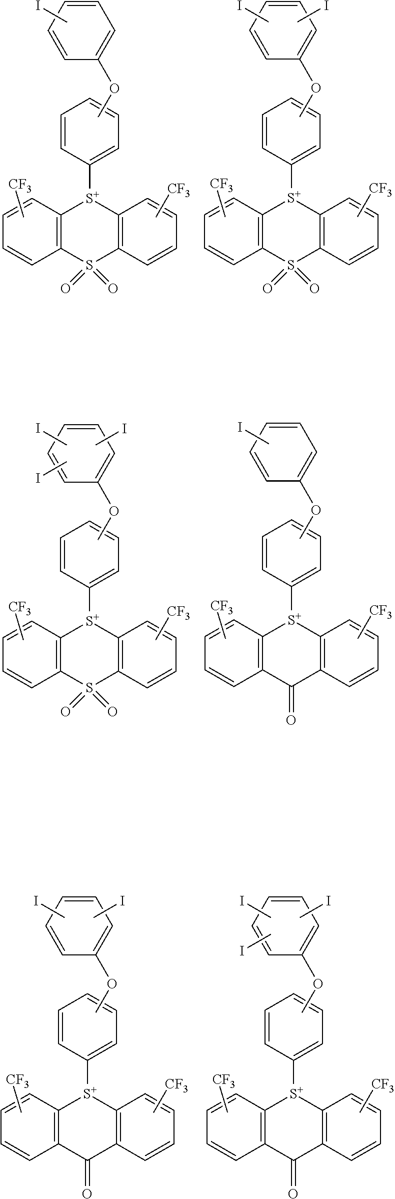

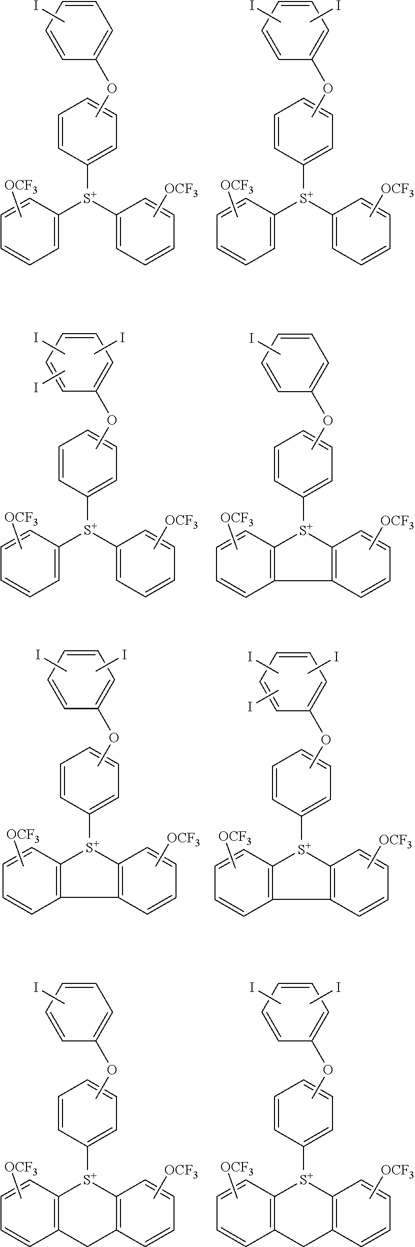

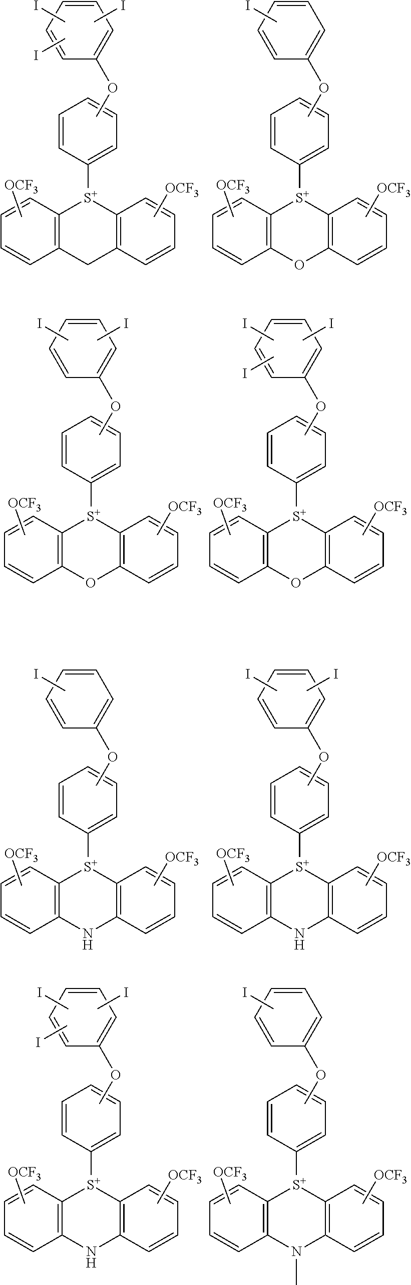

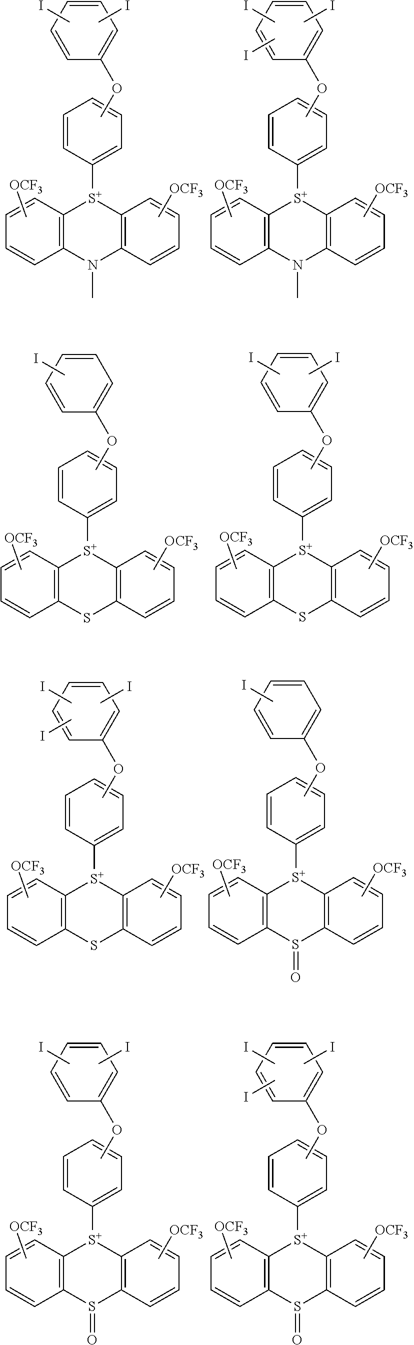

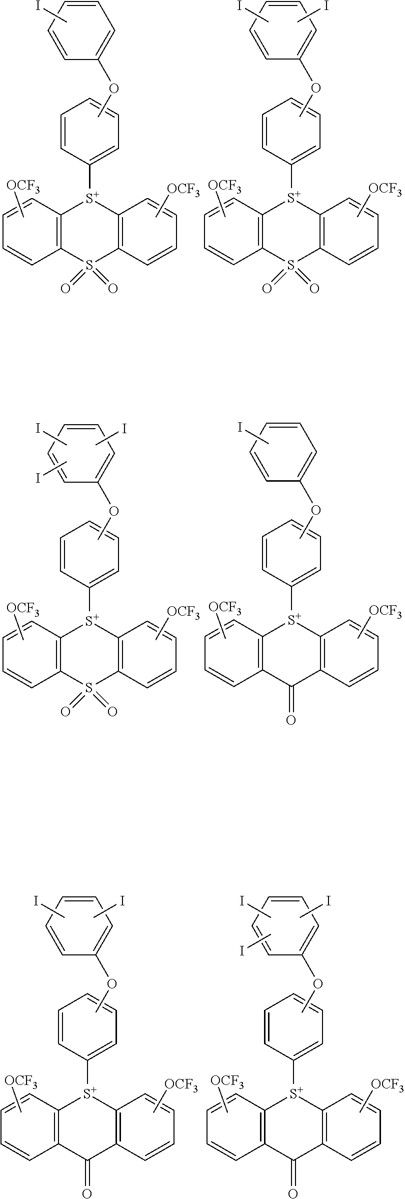

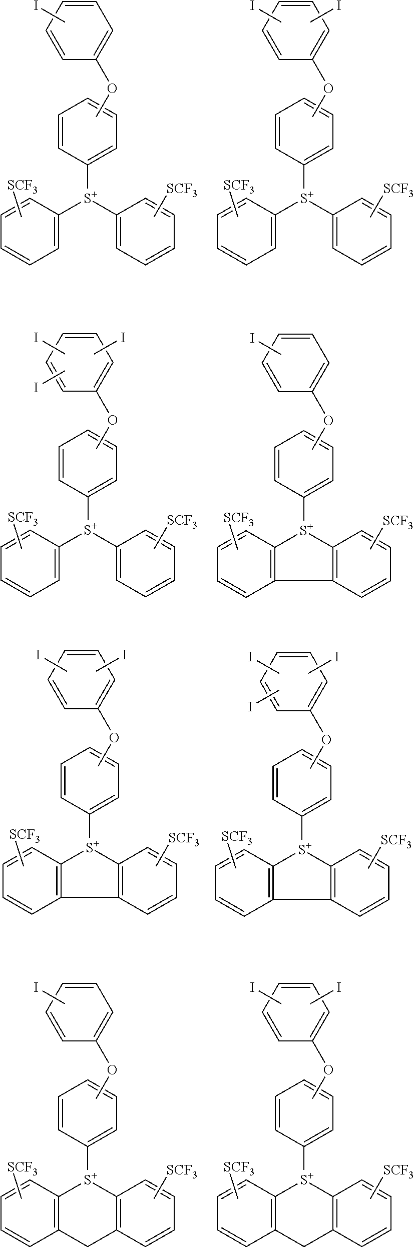

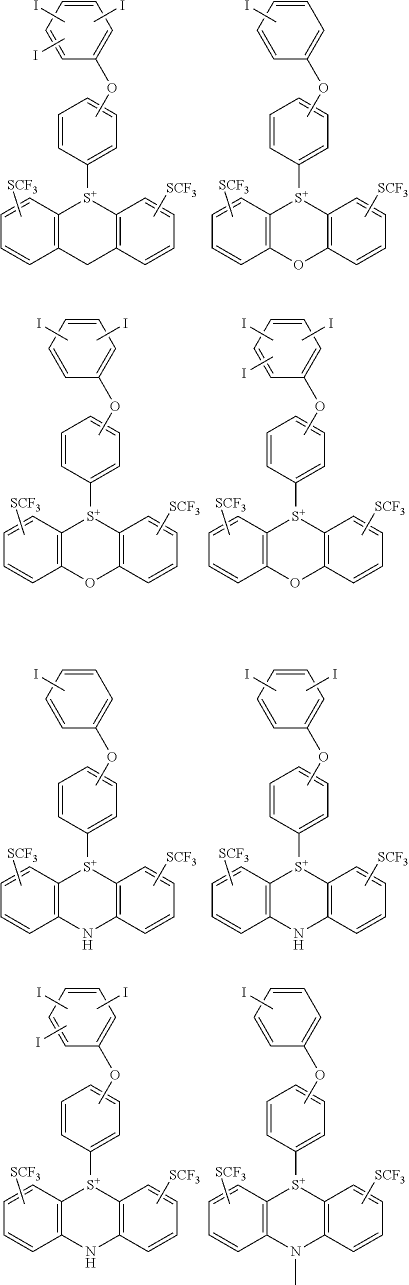

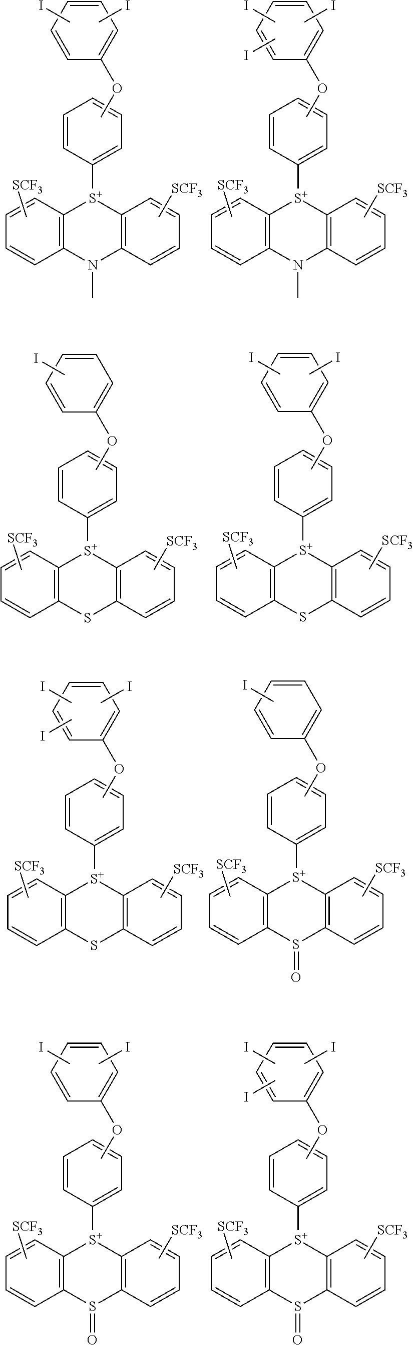

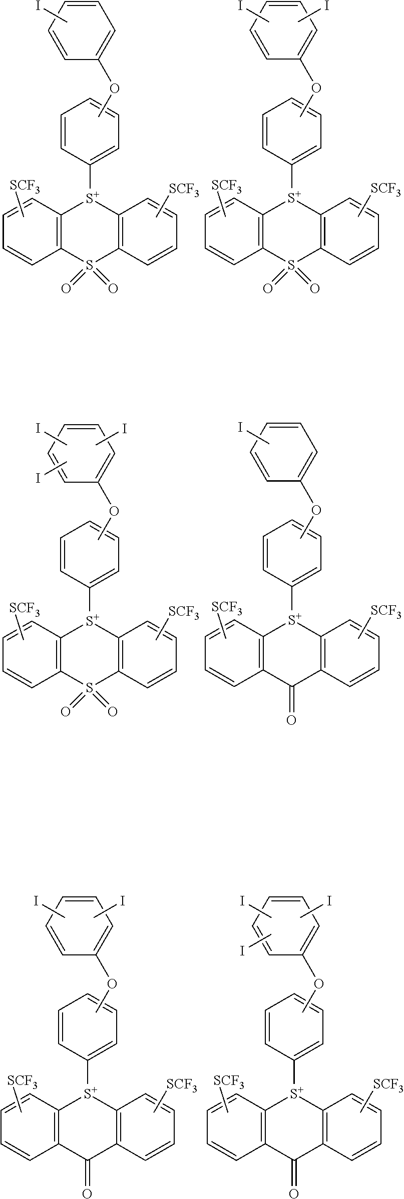

The repeating units h1 to h4 have the function of a photo-acid generator, so when a base polymer having these repeating units is used, the blending of an additive-type photo-acid generator described later, can be omitted.

In the polymer in the present invention, the content ratios of the repeating units b1, b2, b3, P-1, “d”, “e”, “f”, “g”, and “h” are preferably 0<b1≤0.8, 0≤b2≤0.8, 0≤b3≤0.5, 0<P−1≤0.6, 0≤d≤0.6, 0≤e≤0.3, 0≤f≤0.3, 0≤g≤0.3, and 0≤h≤0.4, provided that b1+b2+b3+(P-1)+d+e+f+g+h≤1.0.

The polymer preferably has a weight-average molecular weight (Mw) of 1,000 to 500,000, more preferably 3,000 to 100,000, and further preferably 3,000 to 15,000. The Mw within this range yields sufficient etching resistance, and has no risk of deterioration in resolution caused by failure to achieve a difference in a dissolution rate before and after the exposure. The Mw in the present invention is a measurement value in terms of polystyrene by gel permeation chromatography (GPC) using tetrahydrofuran (THF) or N,N-dimethylformamide (DMF) as a solvent. GPC measurement is usually performed at room temperature of about 23° C., but may be performed at a higher or lower temperature.

Since influence of a molecular weight distribution (Mw/Mn) of the polymer is likely to becomes larger as the pattern rule becomes finer, the Mw/Mn has preferably a narrow distribution ranging from 1.0 to 2.0 to obtain a resist composition suitably used for a fine pattern size. Within the above range, polymers having a smaller molecular weight and a larger molecular weight are reduced, and there is no risk of a foreign matter on a pattern and deterioration in a pattern shape after the exposure.

There are no particular limitations on the amount of the polymer blended in the inventive resist composition, but it can be, for example, 1 to 50 parts by mass, preferably 1 to 20 parts by mass, based on 100 parts by mass of the total amount of the composition.

Examples of methods to synthesize the polymer include a method in which monomers to yield the aforementioned repeating units was added with a radical polymerization initiator in an organic solvent and heated for polymerization.

Specific examples of the organic solvent used in the polymerization include toluene, benzene, THF, diethyl ether, dioxane, cyclohexane, cyclopentane, methyl ethyl ketone (MEK), propylene glycol monomethyl ether acetate (PGMEA), propylene glycol monomethyl ether (PGME), and γ-butyrolactone (GBL). Specific examples of the polymerization initiator include 2,2′-azobisisobutyronitrile (AIBN), 2,2′-azobis(2,4-dimethylvaleronitrile), dimethyl 2,2-azobis(2-methylpropionate), 1,1′-azobis(1-acetoxy-1-phenylethane), benzoyl peroxide, and lauroyl peroxide. The addition amount of these initiators is preferably 0.01 to 25 mol % relative to the total of the monomers to be polymerized. The reaction temperature is preferably 50 to 150° C., and more preferably 60 to 100° C. The reaction time is preferably 2 to 24 hours, and more preferably 2 to 12 hours from the viewpoint of production efficiency. The concentration of the polymerization liquid (weight of all monomer components/weight of all component mixed liquid) is preferably 30% to 50%, and more preferably 35% to 45%.

The polymerization initiator may be added into a solution of the monomers and fed into a reaction vessel, or an initiator solution is prepared separately from the monomer solution and each of the solutions may be independently fed into a reaction vessel. Since a radical generated from the initiator may proceed the polymerization reaction during the waiting time to generate a polymer having an ultra-high molecular weight, the monomer solution and the initiator solution are preferably each independently prepared and added dropwise from the viewpoint of quality control. The acid-labile group may be introduced into the monomer to be used as it is, or may be protected or partially protected after the polymerization. To regulate the molecular weight, chain transfer agents, such as dodecyl mercaptan, 2-mercaptoethanol, thioglycolic acid, methyl thioglycolate, and tert-butyl thioglycolate may be used in combination. In this case, the addition amount of these chain transfer agents is preferably 0.01 to 20 mol % relative to the total of the monomers to be polymerized.

When the monomer has a hydroxy group, the hydroxy group may be substituted with an acetal group that is easily deprotected by an acid such as an ethoxyethyl group during the polymerization, and the protected hydroxy group may be deprotected by a weak acid and water after the polymerization. Alternatively, the hydroxy group may be substituted with an acetyl group, a formyl group, a pivaloyl group, etc. to be subjected to alkaline hydrolysis after the polymerization.

When hydroxystyrene or hydroxyvinylnaphthalene is copolymerized, hydroxystyrene or hydroxyvinylnaphthalene and the other monomers may be heat-polymerized in the organic solvent with adding the radical polymerization initiator. Alternatively, acetoxystyrene or acetoxyvinylnaphthalene may be used, and the acetoxy group is deprotected with alkaline hydrolysis after the polymerization to be converted into polyhydroxystyrene or polyhydroxyvinylnaphthalene.

As specific examples of a base in the alkaline hydrolysis, it is possible to use aqueous ammonia, triethylamine, etc. The reaction temperature is preferably −20 to 100° C., and more preferably 0 to 60° C. The reaction time is preferably 0.2 to 100 hours, and more preferably 0.5 to 20 hours.

The amount of each monomer in the monomer solution is appropriately set so as to be a preferable content rate of the above repeating units, for example.

With the polymer obtained in the manufacturing method, a reaction solution obtained by the polymerization reaction may be a final product. Alternatively, a powder obtained via a purification step, such as reprecipitation method in which the polymerization solution is added into a poor solvent to obtain a powder, may be treated as a final product. From the viewpoints of operation efficiency and quality stabilization, the powder obtained in the purification step is preferably dissolved in a solvent for forming a polymer solution to be operated as a final product.

Specific examples of the solvent used in this case include solvents described in paragraphs [0144] to [0145] of JP2008-111103A, and specifically include: ketones, such as cyclohexanone and methyl-2-n-pentylketone; alcohols, such as 3-methoxybutanol, 3-methyl-3-methoxybutanol, 1-methoxy-2-propanol, and 1-ethoxy-2-propanol; ethers, such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters, such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones, such as GBL; alcohols, such as diacetone alcohol (DAA); alcoholic solvents having a high boiling point, such as diethylene glycol, propylene glycol, glycerin, 1,4-butanediol, and 1,3-butanediol; and a mixed solvent thereof.

In the polymer solution, a concentration of the polymer is preferably 0.01 to 30 mass %, and more preferably 0.1 to 20 mass %.

The reaction solution and the polymer solution are preferably filtered with a filter. The filtration can remove a foreign matter and gel, which may cause a defect, and is effective in terms of quality stabilization.

Examples of a material of the filter used for the filtration include a fluorocarbon, a cellulose, a nylon, a polyester, and a hydrocarbon. In the step of filtering the chemically-amplified resist composition, the filter is preferably formed with a fluorocarbon, so-called Teflon(R), a hydrocarbon such as polyethylene and polypropylene, or nylon. A pore size of the filter can be appropriately selected according to target cleanliness, and is preferably 100 nm or smaller, and more preferably 20 nm or smaller. These filters may be used singly, or may be used in combination of a plurality of these filters. With the filtration method, the solution may be passed through the filter once, but the solution is more preferably circulated to be filtered a plurality of times. In the step for producing the polymer, the filtration step may be performed in any order and times, but the reaction solution after the polymerization reaction, the polymer solution, or both thereof are preferably filtered.

The polymer may be used singly, or may be used in combination of two or more kinds thereof having different composition ratio, Mw, and/or Mw/Mn.

### [(D) Organic Solvent]

The inventive chemically-amplified resist composition may contain an organic solvent as the component (D).

The organic solvent is not particularly limited as long as it can dissolve each component described above and each component described later. Examples of such an organic solvent include: ketones, such as cyclopentanone, cyclohexanone, and methyl-2-n-pentyl ketone; alcohols, such as 3-methoxybutanol, 3-methyl-3-methoxybutanol, 1-methoxy-2-propanol, and 1-ethoxy-2-propanol; keto alcohols, such as DAA; ethers, such as PGME, ethylene glycol monomethyl ether, propylene glycol monoethyl ether, ethylene glycol monoethyl ether, propylene glycol dimethyl ether, and diethylene glycol dimethyl ether; esters, such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones, such as GBL; and a mixed solvent thereof.

Among these organic solvents, preferable solvents are 1-ethoxy-2-propanol, PGMEA, cyclohexanone, GBL, DAA, ethyl lactate, and a mixed solvent thereof, which have particularly excellent solubility of the base polymer of (component A) or (component A-1).

In the inventive chemically-amplified resist composition, the organic solvent (D) is preferably contained in 200 to 5,000 parts by mass, more preferably 400 to 3,500 parts by mass, based on 80 parts by mass of the base polymer (component A) or (component A-1). One kind of the organic solvent (D) may be used, or two or more kinds thereof may be mixed and used.

[(C-2) Acid Diffusion Inhibitor] The inventive chemically-amplified resist composition may contain an acid diffusion inhibitor (component C-2) other than the sulfonium salt (component C-1) represented by the general formula (1). In the present invention, the acid diffusion inhibitor refers to a material that traps the acid generated by the photo-acid generator in the chemically-amplified resist composition, thereby preventing the acid from diffusing into an unexposed portion and forming a desired pattern.

Specific examples of the acid diffusion inhibitor (C-2) include onium salts represented by the following formula (1′) or (2).

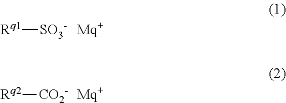

In the formula (1′), R^{q} represents a hydrogen atom or hydrocarbyl group having 1 to 40 carbon atoms and optionally having a heteroatom, except for a group in which a hydrogen atom bonded to a carbon atom at the α-position of the sulfo group is substituted with a fluorine atom or a fluoroalkyl group. In the formula (2), R^{q2} represents a hydrogen atom or hydrocarbyl group having 1 to 40 carbon atoms and optionally having a heteroatom.

Specific examples of the hydrocarbyl group represented by R^{q1} and having 1 to 40 carbon atoms include: alkyl groups having 1 to 40 carbon atoms, such as a methyl group, an ethyl group, an n-propyl group, an isopropyl group, an n-butyl group, isobutyl group, an sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decyl group, and an adamantyl group; and aryl groups having 6 to 40 carbon atoms, such as a phenyl group, a naphthyl group, and an anthracenyl group. A part or all of hydrogen atoms in the hydrocarbyl group are optionally substituted with a group having a heteroatom, such as an oxygen atom, a sulfur atom, a nitrogen atom, and a halogen atom. A part of “—CH₂—” in the hydrocarbyl group is optionally substituted with a group having a heteroatom, such as an oxygen atom, a sulfur atom, and a nitrogen atom. As a result, the hydrocarbyl group may have a hydroxy group, fluorine atom, chlorine atom, bromine atom, iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

Specific examples of the hydrocarbyl group represented by R^{q2} include: the substituents exemplified as the specific examples of R^{q1}; fluorinated saturated hydrocarbyl groups, such as a trifluoromethyl group and a trifluoroethyl group; and fluorinated aryl groups, such as a pentafluorophenyl group and a 4-trifluoromethylphenyl group.

Specific examples of the anion of the onium salt represented by formula (1′) include the anions shown below, but are not limited thereto.

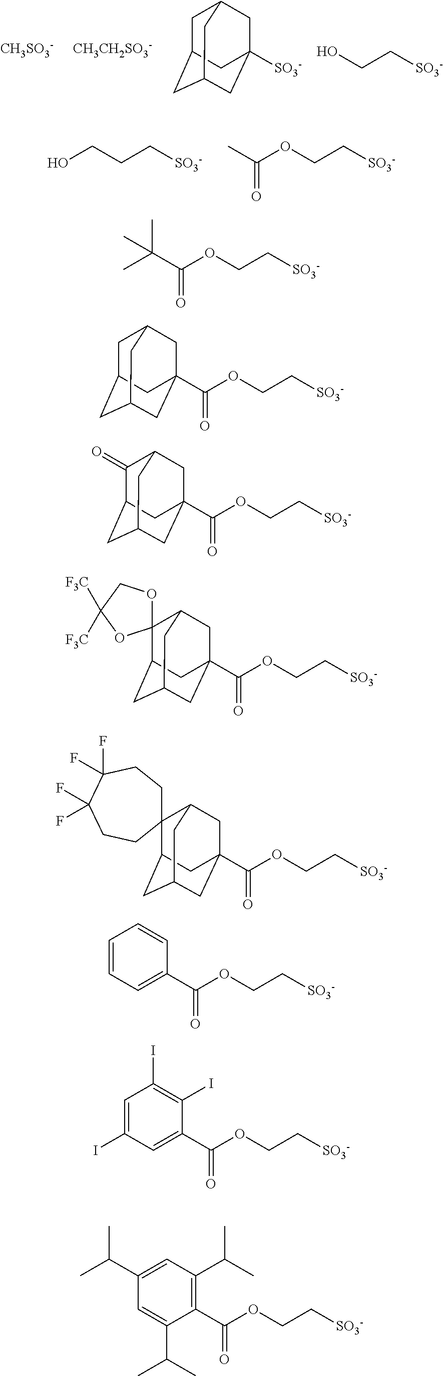

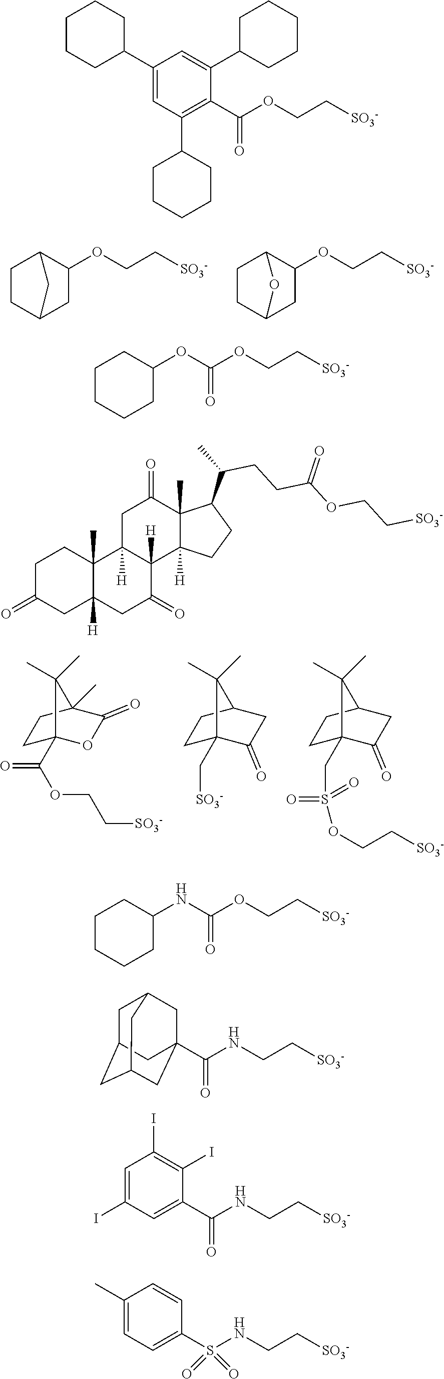

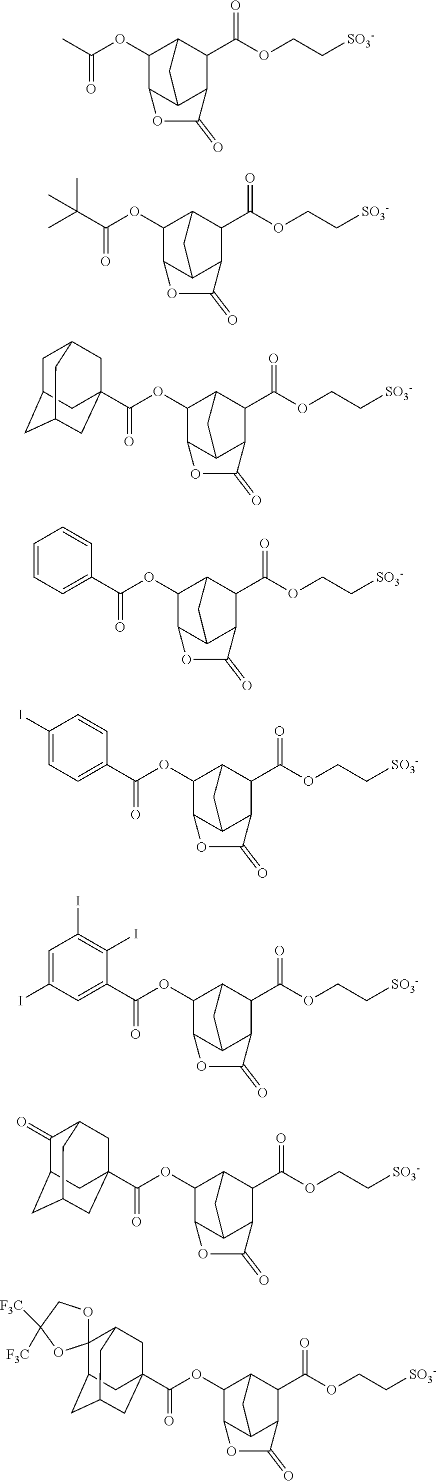

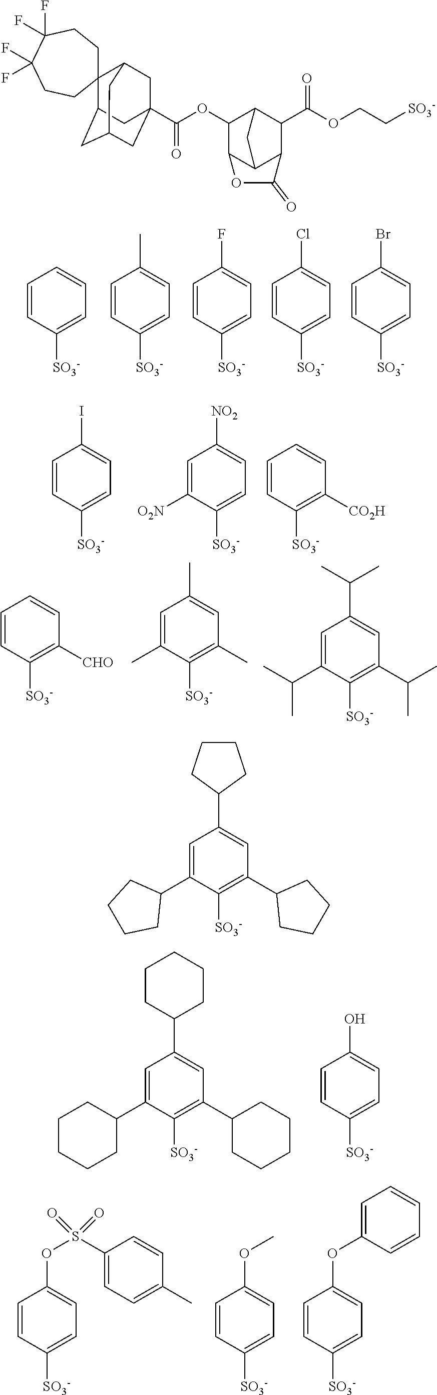

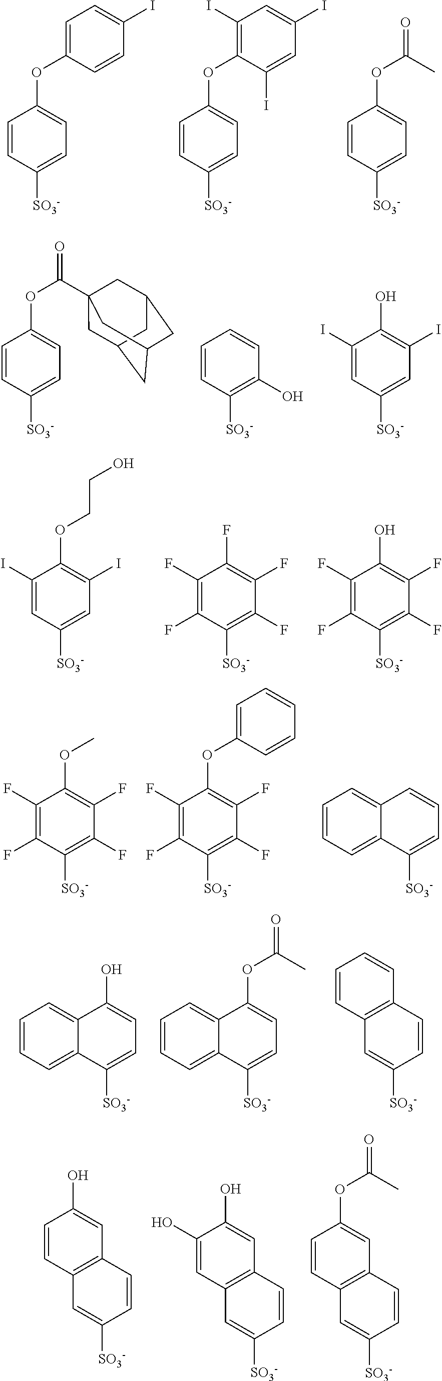

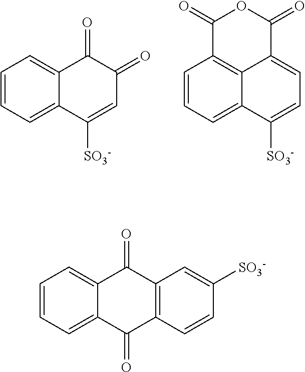

Specific examples of the anion of the onium salt represented by formula (2) include the anions shown below, but are not limited thereto.

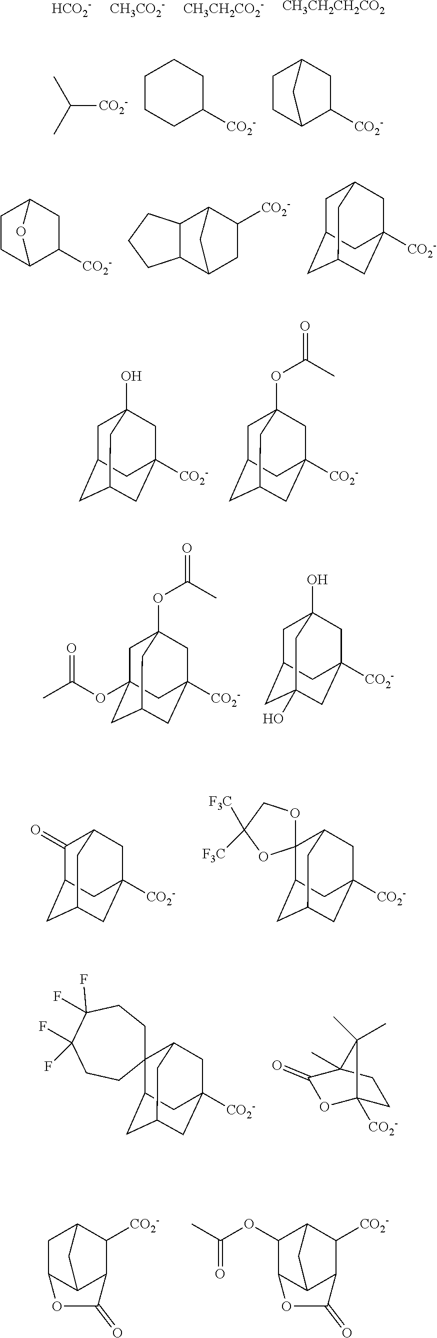

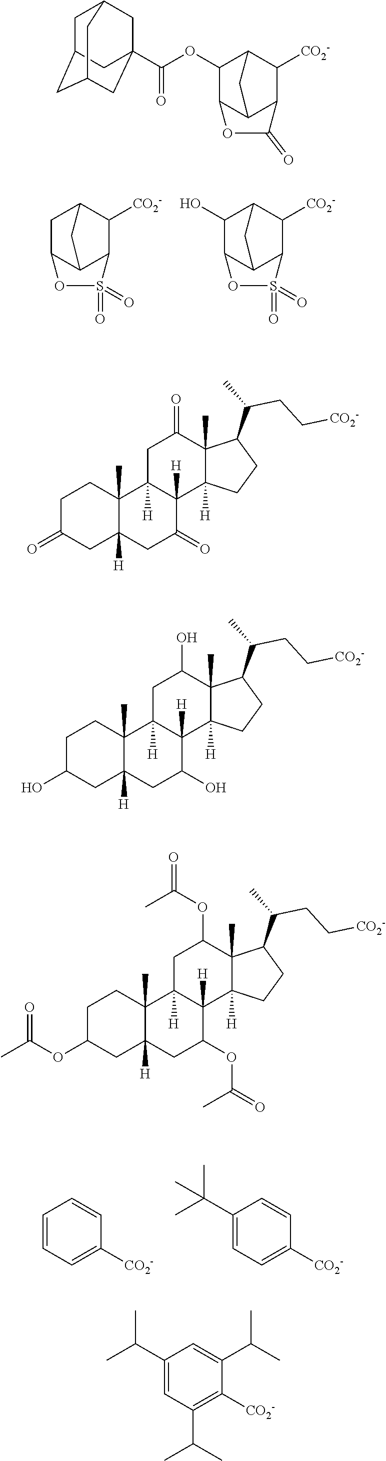

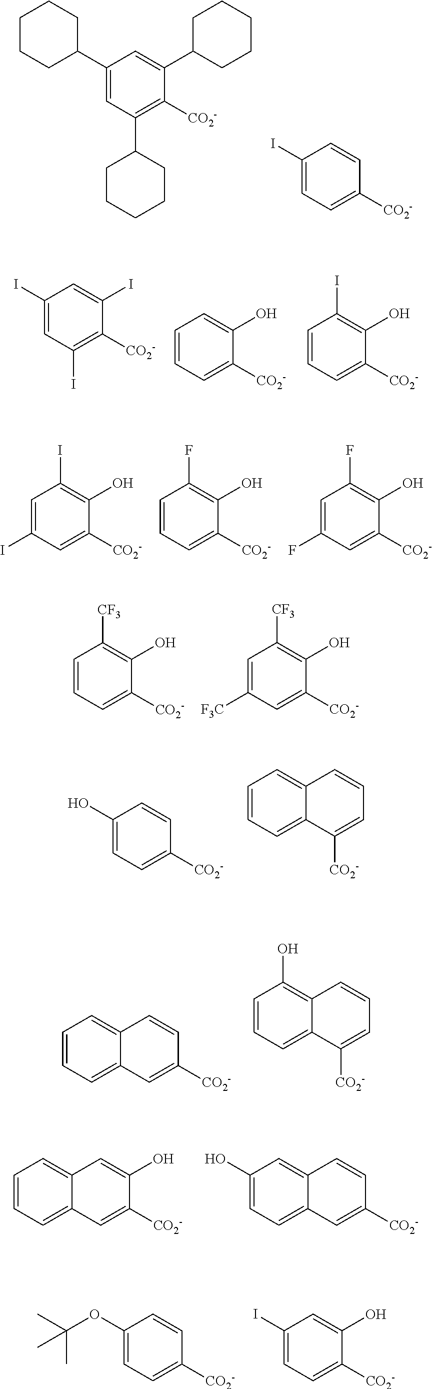

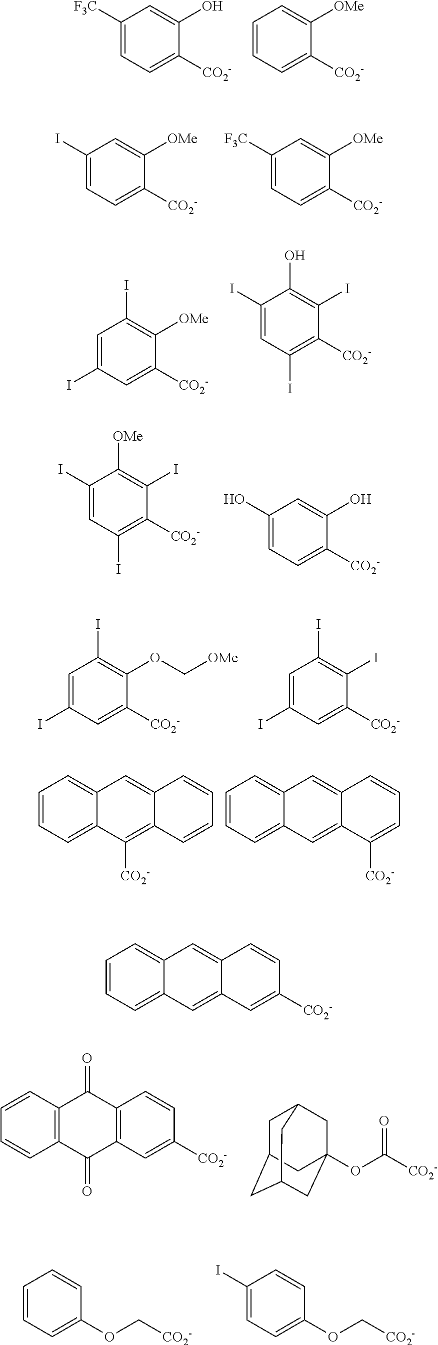

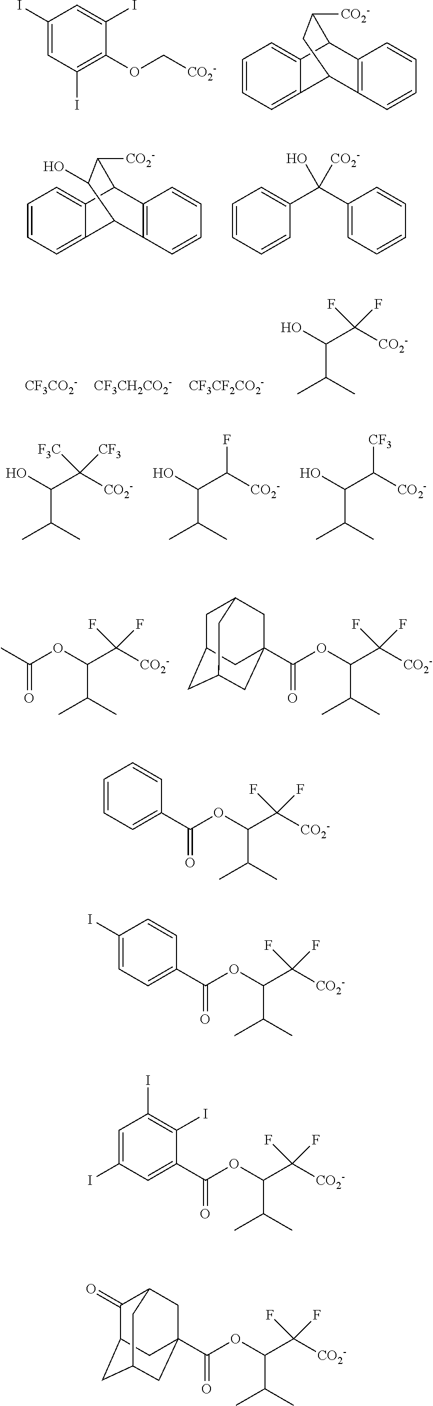

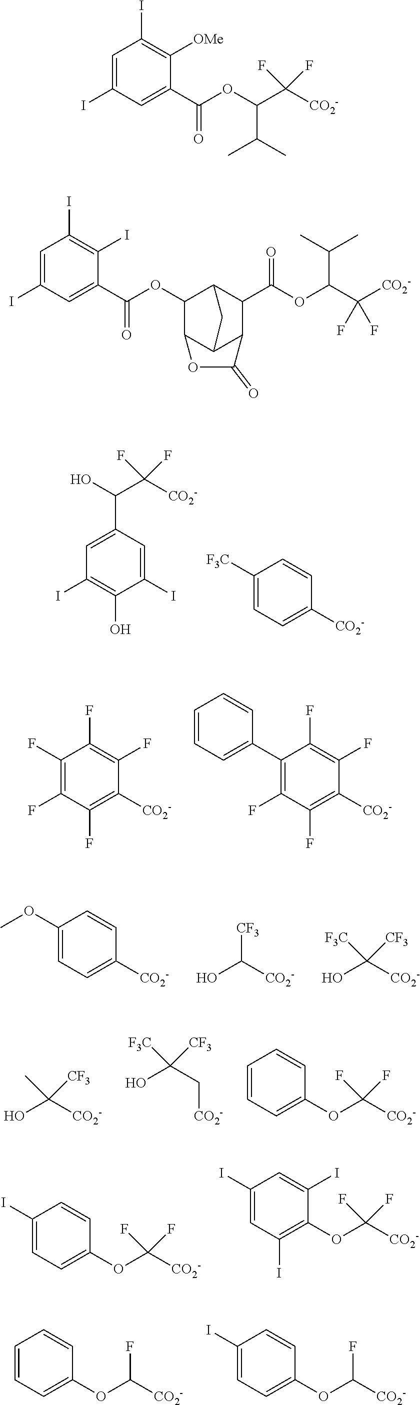

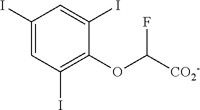

In the formulae (1′) and (2), Mq⁺ is an onium cation. The onium cation is preferably a sulfonium cation in the repeating units h2 to h4 described above, a substituted or unsubstituted diphenyliodonium cation, or an ammonium cation represented by the following formula (cation-3).

Examples of the substituted or unsubstituted diphenyliodonium cation include those having the structures shown below.

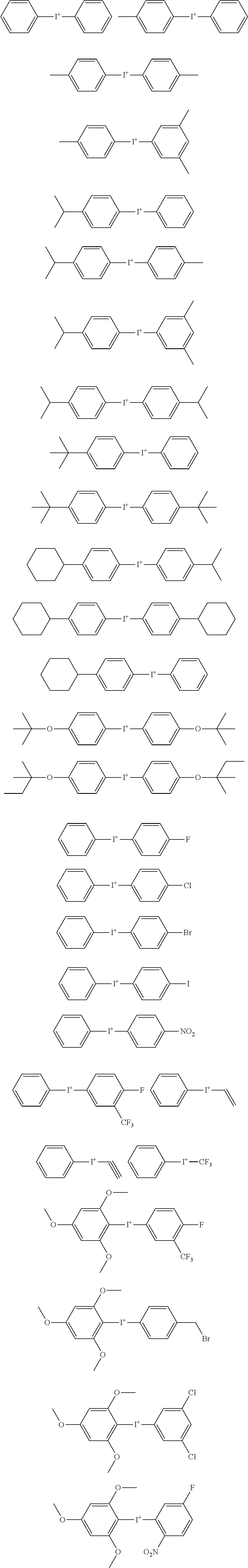

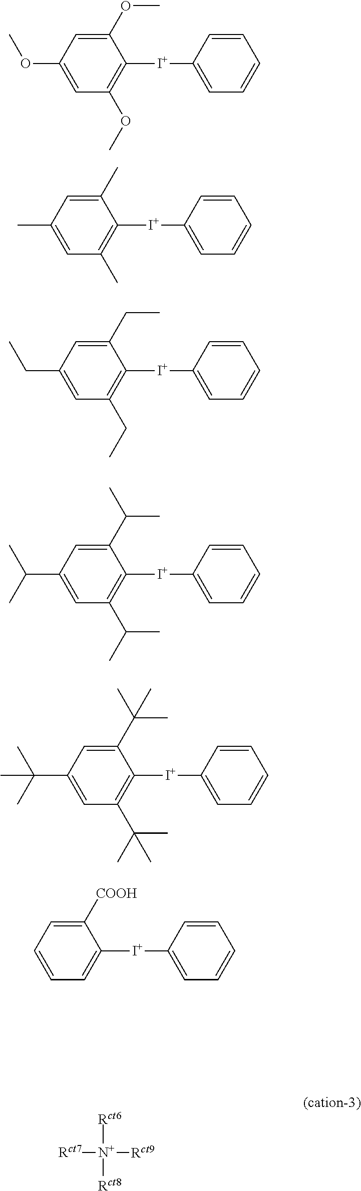

In the formula (cation-3), R^{ct6} to R^{ct9} each independently represent a hydrocarbyl group having 1 to 40 carbon atoms and optionally having a heteroatom. In addition, two or more of R^{ct6}, R^{ct7}, and R^{ct8} may be bonded to each other to form a ring together with the nitrogen atom to which they are bonded. Specific examples of the hydrocarbyl group include the same as those exemplified as the hydrocarbyl groups represented by R³¹ to R⁴¹ in description of the formulae (h1) to (h4).

Specific examples of the ammonium cation represented by formula (cation-3) include those shown below, but are not limited thereto.

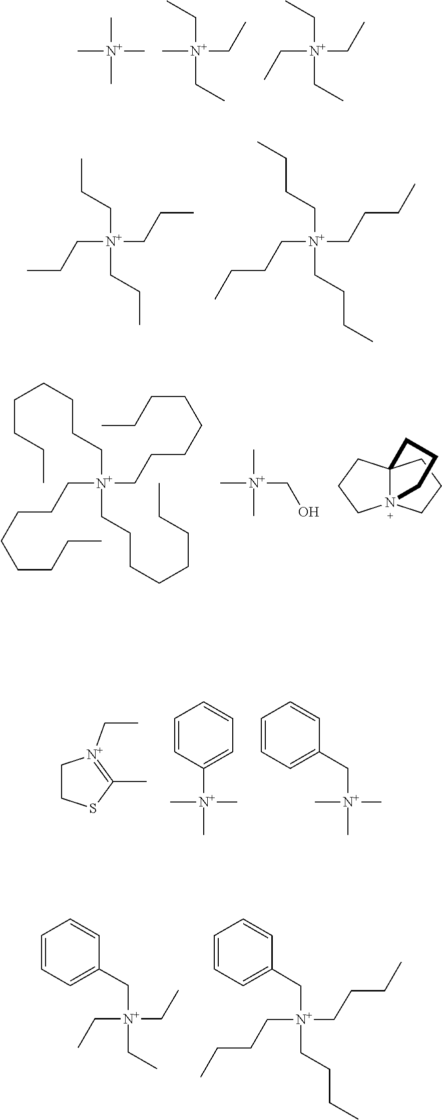

Specific examples of the onium salt represented by formula (1′) or (2) include any combination of the above-mentioned anions and cations. These onium salts can be easily prepared by ion exchange reactions using known organic chemical methods. For information on the ion exchange reactions, for example, JP2007-145797A can be referred.

Examples of the acid diffusion inhibitor (C-2) other than the onium salt represented by formula (1′) or (2) include a combination of imide anion or methide acid anion shown below and the onium cation described above as Mq⁺.

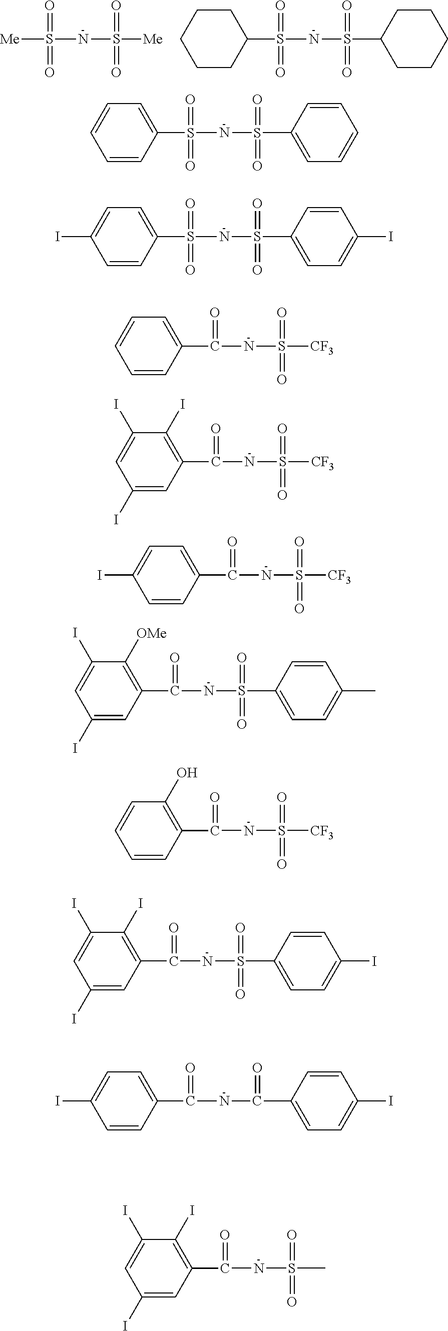

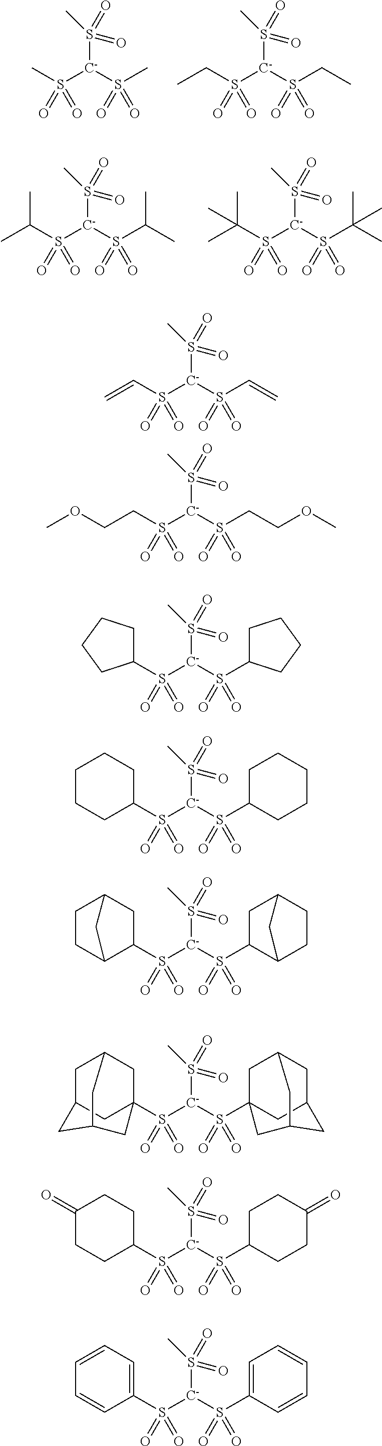

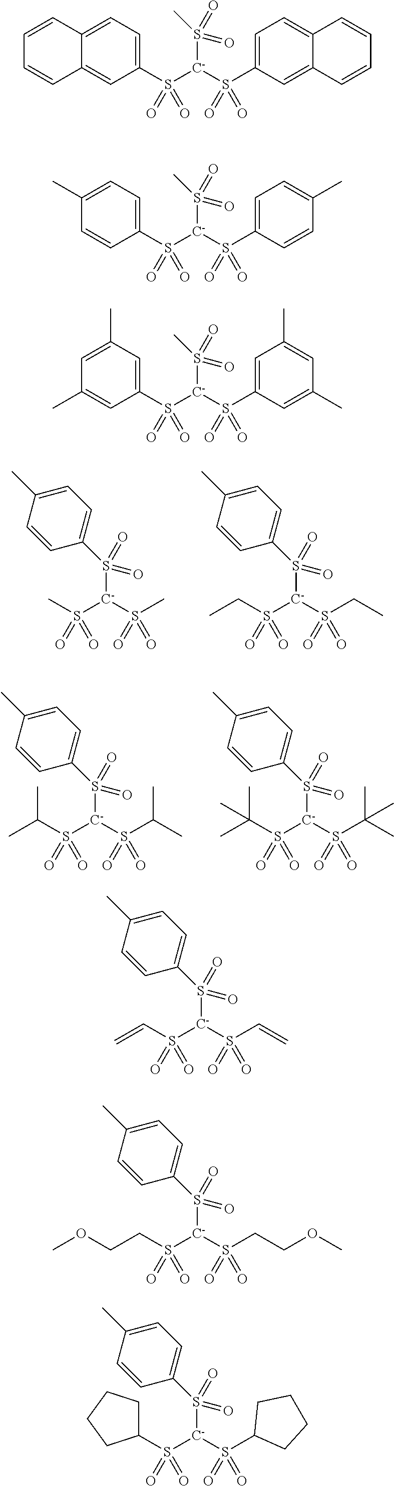

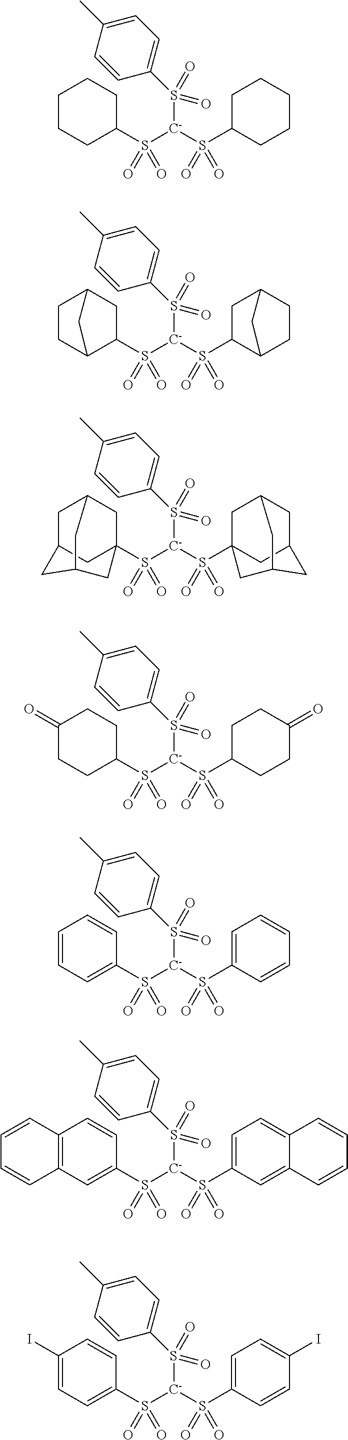

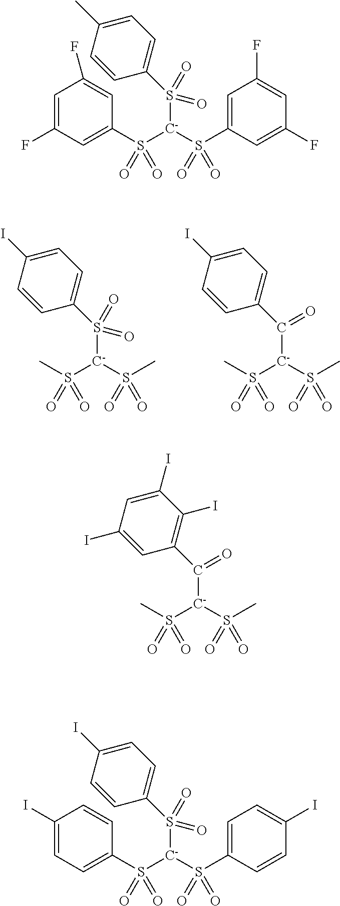 [SMILES: Cc1ccc(S(=O)(=O)[C-](S(=O)(=O)c2cc(F)cc(F)c2)S(=O)(=O)c2cc(F)cc(F)c2)cc1 ; CS(=O)(=O)[C-](S(C)(=O)=O)S(=O)(=O)c1ccc(I)cc1 ; CS(=O)(=O)[C-](C(=O)c1ccc(I)cc1)S(C)(=O)=O ; CS(=O)(=O)[C-](C(=O)c1cc(I)cc(I)c1I)S(C)(=O)=O ; O=S(=O)(c1ccc(I)cc1)[C-](S(=O)(=O)c1ccc(I)cc1)S(=O)(=O)c1ccc(I)cc1]

The onium salt represented by the formulae (1′), (2), and (C-2) acts as a quencher in the inventive chemically-amplified resist composition. This is because each counter anion of the onium salt is a conjugated base of a weak acid. The weak acid herein means an acid exhibiting acidity that cannot deprotect the acid-labile group in the acid-labile group-containing unit used for the base polymer. The onium salt represented by the formulae (1′), (2), and (C-2) functions as a quencher when used in combination with an onium-salt type photo-acid generator having a conjugated base of a strong acid, such as an a-fluorinated sulfonic acid, as a counter anion. That is, when an onium salt to generate a strong acid, such as an a-fluorinated sulfonic acid, and an onium salt to generate a weak acid, such as non-fluorinated sulfonic acid and a carboxylic acid, are mixed to be used, the strong acid generated from the photo-acid generator by high-energy ray irradiation collides the unreacted onium salt having the weak acid anion to release the weak acid with salt exchange, resulting in generation of an onium salt having the strong acid anion. This process exchanges the strong acid into the weak acid having lower catalytic ability, and the acid is apparently deactivated to enable to control the acid diffusion.

Usable for the acid diffusion inhibitor (C-2) are: an onium salt having a sulfonium cation moiety and a phenoxide anion moiety in the same molecule, described in JP6848776B; an onium salt having a sulfonium cation moiety and a carboxylate anion moiety in the same molecule, described in JP6583136B and JP2020-200311A; and an onium salt having an iodonium cation moiety and a carboxylate anion moiety in the same molecule, descried in JP6274755B.

When the photo-acid generator to generate the strong acid is an onium salt, the strong acid generated by high-energy beam irradiation can be exchanged into the weak acid, as described above. Meanwhile, it is considered difficult for the weak acid generated by high-energy beam irradiation hardly to collide the unreacted onium salt to generate the strong acid to cause salt exchange. This is because of a phenomenon that an onium cation is more likely to form an ion pair with an anion of a stronger acid.

When the inventive chemically-amplified resist composition contains the onium salt represented by the formulae (1′), (2), and (C-2), the content thereof is preferably 0.1 to 25 parts by mass, and more preferably 0.1 to 15 parts by mass, relative to 80 parts by mass of the base polymer (component A) or (component A-1). When the content of the onium-salt type quencher is in the above range, it is preferable in terms of good resolution without considerable deterioration in the sensitivity. One kind of these components may be used, or two or more kinds thereof may be used in combination.

The inventive chemically-amplified resist composition may further comprise a nitrogen-containing compound as the component (C-2) acid diffusion inhibitor. Specific examples of the nitrogen-containing compound include primary, secondary, or tertiary amine compounds described in paragraphs [0146] to [0164] of JP2008-111103A, in particular, amine compounds having a hydroxy group, an ether bond, an ester bond, a lactone ring, a cyano group, or a sulfonate ester bond. Additionally, specific examples thereof also include compounds in which a primary or secondary amine is protected with a carbamate group, as compounds described in JP3790649B.

As the nitrogen-containing compound, a sulfonium sulfonate salt having a nitrogen-containing substituent may also be used. Such a compound functions as a quencher in an unexposed portion, and loses the quenching ability in an exposed portion by neutralization with a generated acid of its own, to function as a so-called photodegradable base. Using the photodegradable base can further enhance the contrast between the exposed portion and the unexposed portion. For the photodegradable base, JP2009-109595A and JP2012-46501A can be referred, for example.

When the inventive chemically-amplified resist composition contains a nitrogen-containing compound as the component (C) acid diffusion inhibitor, its content thereof is preferably 0.001 to 12 parts by mass, and more preferably 0.01 to 8 parts by mass, relative to 80 parts by mass of the base polymer (component A) or (component A-1). One kind of the nitrogen-containing compound may be used, or two or more kinds thereof may be used in combination.

### [(B) Photo-Acid Generator]

When the inventive chemically-amplified resist composition contains a resin (component A) as a base polymer, the resist composition also contains a photo-acid generator (component B). However, when the inventive chemically-amplified resist composition contains a resin (component A-1) as a base polymer, the photo-acid generator (component B) is not necessarily an essential component. Examples of the photo-acid generator include a compound that generates an acid in response to an active ray or radiation (photo-acid generator). The photo-acid generator is not particularly limited as long as it is a compound that generates an acid when irradiated with a high-energy beam, but is preferably those which generate sulfonic acids, imide acids or methide acids. Suitable photo-acid generators include sulfonium salts, an iodonium salts, sulfonyldiazomethane, N-sulfonyloxyimide, and oxime-O-sulfonate type acid generators. Specific examples of the acid generators include those described in paragraphs [0122] to [0142] of JP2008-111103A.

As the photo-acid generator, it is also possible to use suitably a sulfonium salt represented by the following formula (3-1) or an iodonium salt represented by the following formula (3-2).

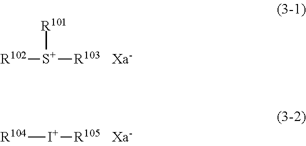

In the formulae (3-1) and (3-2), R¹⁰¹ to R¹⁰⁵ each independently represent a hydrocarbyl group having 1 to 20 carbon atoms and optionally having a heteroatom. The hydrocarbyl group may be a saturated or unsaturated group, and may be linear, branched, or cyclic. Specific examples thereof include: alkyl groups, such as a methyl group, an ethyl group, an n-propyl group, an isopropyl group, an n-butyl group, and a tert-butyl group; cyclic saturated hydrocarbyl groups, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups, such as a vinyl group, an allyl group, a propenyl group, a butenyl group, and a hexenyl group; cyclic unsaturated aliphatic hydrocarbyl groups, such as a cyclohexenyl group; aryl groups, such as a phenyl group and a naphthyl group; heteroaryl groups, such as a thienyl group; and aralkyl groups, such as a benzyl group, a 1-phenylethyl group, and a 2-phenylethyl group. Among these, it is preferably an aryl group. A part or all of hydrogen atoms of these groups may be substituted with a group having a heteroatom, such as an oxygen atom, a sulfur atom, a nitrogen atom, and a halogen atom. A part of these groups may be substituted with a group having a heteroatom such as an oxygen atom, a sulfur atom, and a nitrogen atom. As a result, the resulting hydrocarbyl group may have a hydroxy group, a cyano group, a carbonyl group, an ether bond, an ester bond, a sulfonate ester bond, a carbonate bond, a lactone ring, a sultone ring, a carboxylic anhydride, a haloalkyl group, etc. In addition, R¹⁰¹ and R¹⁰² may be bonded to each other to form a ring together with the sulfur atom to which they are bonded.

Specific examples of the cation of the sulfonium salt represented by the formula (3-1) include the same as those exemplified as the sulfonium cation in the repeating units h2 to h4 described above. Specific examples of the cation of the iodonium salt represented by formula (3-2) include the same as those exemplified as the diphenyliodonium cation in the component C-2 described above.

In the formulae (3-1) and (3-2), Xa⁻ is an anion selected from the following formulae (3A) to (3D).

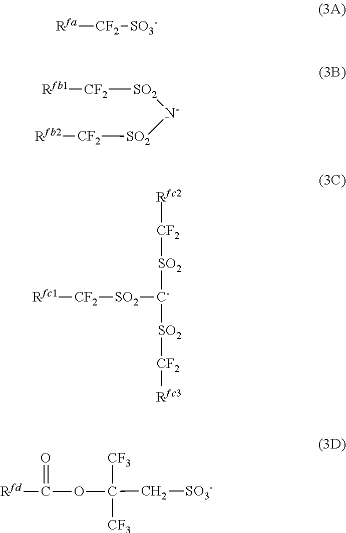

In the formula (3A), R^{fa} represents a fluorine atom or a hydrocarbyl group having 1 to 40 carbon atoms and optionally having a heteroatom. The hydrocarbyl group may be a saturated or unsaturated group, and may be linear, branched, or cyclic. Specific examples thereof include same groups as those exemplified in the description for Ral in the formula (3A′) described later.

The anion represented by the formula (3A) is preferably represented by the following formula (3A′).

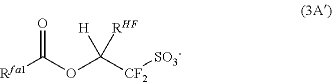

In the formula (3A′), R^{HF} represents a hydrogen atom or a trifluoromethyl group, and preferably a trifluoromethyl group.

In the formula (3A′), R^{fa1} represents a hydrocarbyl group having 1 to 38 carbon atoms and optionally having a heteroatom. The heteroatom is preferably an oxygen atom, a nitrogen atom, a sulfur atom, a halogen atom, or the like, and more preferably an oxygen atom. The hydrocarbyl group particularly preferably has 6 to 30 carbon atoms from the viewpoint of obtaining high resolution in fine pattern formation.

The hydrocarbyl group having 1 to 38 carbon atoms and represented by R^{fa1} may be a saturated or unsaturated group, and may be linear, branched, and cyclic. Specific examples thereof include: alkyl groups having 1 to 38 carbon atoms, such as a methyl group, an ethyl group, an n-propyl group, an isopropyl group, an n-butyl group, an isobutyl group, an sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosyl group; cyclic saturated hydrocarbyl groups having 3 to 38 carbon atoms, such as a cyclopentyl group, a cyclohexyl group, a 1-adamantyl group, a 2-adamantyl group, a 1-adamantylmethyl group, a norbornyl group, a norbornylmethyl group, a tricyclodecyl group, a tetracyclododecyl group, a tetracyclododecylmethyl group, and a dicyclohexylmethyl group; unsaturated aliphatic hydrocarbyl groups having 2 to 38 carbon atoms, such as an allyl group and a 3-cyclohexenyl group; aryl groups having 6 to 38 carbon atoms, such as a phenyl group, a 1-naphthyl group, and a 2-naphthyl group; aralkyl groups having 7 to 38 carbon atoms, such as a benzyl group and a diphenylmethyl group; and groups obtained by combining these groups.

A part or all of hydrogen atoms in the hydrocarbyl group may be substituted with a group having a heteroatom, such as an oxygen atom, a sulfur atom, a nitrogen atom, and a halogen atom. A part of “—CH₂—” in the hydrocarbyl group may be substituted with a group having a heteroatom, such as an oxygen atom, a sulfur atom, and a nitrogen atom. As a result, the resulting hydrocarbyl group may have a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc. The heteroatom is preferably an oxygen atom. Specific examples of the hydrocarbyl group having a heteroatom include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and a 3-oxocyclohexyl group.

The synthesis of the sulfonium salt having the anion represented by the formula (3A′) is described in detail in JP2007-145797A, JP2008-106045A, JP2009-7327A, JP2009-258695A, etc. In addition, sulfonium salts disclosed in JP2010-215608A, JP2012-41320A, JP2012-106986A, JP2012-153644A, etc. are also suitably used.

Specific examples of the anion represented by the formula (3A) include the anions shown as specific examples of the formula (h1-1), but are not limited thereto.

In the formula (3B), R^{fb1} and R^{fb2} each independently represent a fluorine atom, or a hydrocarbyl group having 1 to 40 carbon atoms and optionally having a heteroatom. The hydrocarbyl group may be a saturated or unsaturated group, and may be linear, branched, or cyclic. Specific examples thereof include same groups as those exemplified as the hydrocarbyl group represented by R^{fa1} in the formula (3A′). R^{fb1} and R^{fb2} preferably represent a fluorine atom or a linear fluorinated alkyl group having 1 to 4 carbon atoms. R^{fb1} and R^{fb2} may be bonded to each other to form a ring together with a group (—CF₂—SO₂—N—SO₂—CF₂—) to which R^{fb1} and R^{fb2} are bonded. In this case, the group obtained by bonding R^{fb1} and R^{fb2} to each other is preferably a fluorinated ethylene group or a fluorinated propylene group.

In the formula (3C), R^{fc1}, R^{fc2}, and R^{fc3} each independently represent a fluorine atom, or a hydrocarbyl group having 1 to 40 carbon atoms and optionally having a heteroatom. The hydrocarbyl group may be a saturated or unsaturated group, and may be linear, branched, and cyclic. Specific examples thereof include same groups as those exemplified as the hydrocarbyl group represented by R^{fa1} in the formula (3A′). R^{fc1}, R^{fc2}, and R^{fc3} preferably represent a fluorine atom or a linear fluorinated alkyl group having 1 to 4 carbon atoms. R^{fc1} and R^{fc2} are optionally bonded to each other to form a ring together with a group (—CF₂—SO₂—C⁻—SO₂—CF₂—) to which R^{fc1} and R^{fc2} are bonded. In this case, the group obtained by bonding R^{fc1} and R^{fc2} to each other is preferably a fluorinated ethylene group or a fluorinated propylene group.

In the formula (3D), R^{fd} represents a hydrocarbyl group having 1 to 40 carbon atoms and optionally having a heteroatom. The hydrocarbyl group may be a saturated or unsaturated group, and may be linear, branched, or cyclic groups. Specific examples thereof include same groups as those exemplified as the hydrocarbyl group represented by R^{fa1} in the formula (3A′).

The synthesis of the sulfonium salt containing the anion represented by the general formula (3D) is described in detail in JP2010-215608A and JP2014-133723A.

Specific examples of the anion represented by formula (3D) include the anions shown as specific examples of formula (h1-2), but are not limited thereto.

The photo-acid generator containing the anion represented by the formula (3D) does not have a fluorine atom at the a position of the sulfo group, but has two trifluoromethyl groups at the β position, thereby providing sufficient acidity to cut the acid labile group in the base polymer. Thus, it is possible to use as photo-acid generator.

One represented by the following general formula (4) can also be used suitably as a photo-acid generator.

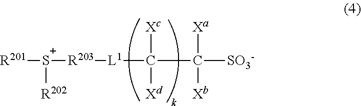

In the general formula (4), R²⁰¹ and R²⁰² each independently represent a hydrocarbyl group having 1 to 30 carbon atoms and optionally having a heteroatom. R²⁰³ represents a hydrocarbylene group having 1 to 30 carbon atoms and optionally having a heteroatom. Any two of R²⁰¹, R²⁰², and R²⁰³ may bond to each other to form a ring together with a sulfur atom bonded to these.

The hydrocarbyl group having 1 to 30 carbon atoms and represented by R²⁰¹ and R²⁰² may be saturated or unsaturated, and may be linear, branched, or cyclic. Specific examples thereof include: alkyl groups having 1 to 30 carbon atoms, such as a methyl group, an ethyl group, a n-propyl group, an isopropyl group, an n-butyl group, an isobutyl group, an sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 30 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, an oxanorbornyl group, a tricyclo[5.2.1.0^{2,6}]decyl group, and an adamantyl group; aryl groups having 6 to 30 carbon atoms, such as a phenyl group, a methylphenyl group, an ethylphenyl group, an n-propylphenyl group, an isopropylphenyl group, an n-butylphenyl group, an isobutylphenyl group, a sec-butylphenyl group, a tert-butylphenyl group, a naphthyl group, a methylnaphthyl group, an ethylnaphthyl group, an n-propylnaphthyl group, an isopropylnaphthyl group, an n-butylnaphthyl group, an isobutylnaphthyl group, a sec-butylnaphthyl group, a tert-butylnaphthyl group, and an anthracenyl group; and groups obtained by combining these. Additionally, a part or all of hydrogen atoms of the hydrocarbyl groups may be substituted with a group containing a heteroatom, such as an oxygen atom, a sulfur atom, a nitrogen atom, and a halogen atom, and a part of “—CH₂—” of the hydrocarbyl groups may be substituted with a group containing a heteroatom, such as an oxygen atom, a sulfur atom, or a nitrogen atom. As a result, the resulting hydrocarbyl group may contain a hydroxy group, a cyano group, a fluorine atom, a chlorine atom, a bromine atom, an iodine atom, a carbonyl group, an ether bond, an ester bond, a sulfonic acid ester group, a carbonate bond, a lactone ring, a sultone ring, carboxylic anhydride (—C(═O)—O—C(═O)—), a haloalkyl group, etc.

The hydrocarbylene group having 1 to 30 carbon atoms and represented by R²⁰³ may be saturated or unsaturated, and may be linear, branched, or cyclic. Specific examples thereof include: alkanediyl groups having 1 to 30 carbon atoms, such as a methanediyl group, an ethane-1,1-diyl group, ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; cyclic saturated hydrocarbylene groups having 3 to 30 carbon atoms, such as a cyclopentanediyl group, a cyclohexanediyl group, a norbornanediyl group, and an adamantanediyl group; arylene groups having 6 to 30 carbon atoms, such as a phenylene group, a methylphenylene group, an ethylphenylene group, an n-propylphenylene group, an isopropylphenylene group, an n-butylphenylene group, an isobutylphenylene group, an sec-butylphenylene group, a tert-butylphenylene group, a naphthylene group, a methylnaphthylene group, an ethylnaphthylene group, an n-propylnaphthylene group, an isopropylnaphthylene group, an n-butylnaphthylene group, an isobutylnaphthylene group, an sec-butylnaphthylene group, and a tert-butylnaphthylene group; and groups obtained by combining these. A part or all of hydrogen atoms of the hydrocarbylene group may be substituted with a group containing a heteroatom, such as an oxygen atom, a sulfur atom, a nitrogen atom, and a halogen atom, and a part of the “—CH₂—” of the hydrocarbylene group may be substituted with a group containing a heteroatom, such as an oxygen atom, a sulfur atom, and a nitrogen atom. As a result, the resulting hydrocarbylene group may have a hydroxy group, a cyano group, a fluorine atom, a chlorine atom, a bromine atom, an iodine atom, a carbonyl group, an ether bond, an ester bond, a sulfonic acid ester group, a carbonate bond, a lactone ring, a sultone ring, carboxylic anhydride (—C(═O)—O—C(═O)—), a haloalkyl group, etc. The heteroatom is preferably an oxygen atom.

In the general formula (4), L¹ represents a single bond, an ether bond, or a hydrocarbylene group having 1 to 20 carbon atoms and optionally containing a heteroatom. The hydrocarbylene group may be saturated or unsaturated, and may be linear, branched, or cyclic. Specific examples thereof include the same as those exemplified as the hydrocarbylene group represented by R²⁰³.

In the formula (4), X^{a}, X^{b}, X^{c}, and X^{d} each independently represent a hydrogen atom, a fluorine atom, or a trifluoromethyl group, provided that at least one of X^{a}, X^{b}, X^{c}, and X^{d} is a fluorine atom or a trifluoromethyl group.

In the formula (4), “k” is an integer from 0 to 3.

The photo-acid generator represented by the formula (4) is preferably represented by the following formula (4′).

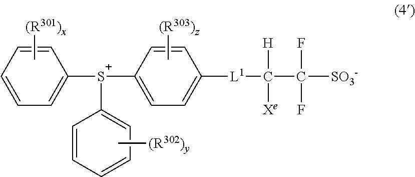

In the general formula (4′), L¹ is the same as defined above. X^{e} represents a hydrogen atom or a trifluoromethyl group, preferably a trifluoromethyl group. R³⁰¹, R³⁰², and R³⁰³ each independently represent a hydrocarbyl group having 1 to 20 carbon atoms and optionally having a heteroatom. The hydrocarbyl group may be saturated or unsaturated, and may be linear, branched, or cyclic. Specific examples thereof include the same as those exemplified as the hydrocarbyl group represented by R^{fa1} in the general formula (3A′). “x” and “y” each independently represent an integer of 0 to 5. “z” represents an integer of 0 to 4.

Specific examples of the photo-acid generator represented by the general formula (4) include the same as those exemplified as a photo-acid generator represented by the formula (2) in JP2017-026980A.

Among the photo-acid generators, the photo-acid generators having the anion represented by the general formula (3A′) or (3D) are particularly preferable because of small acid diffusion and excellent solubility in a solvent. Those represented by the general formula (4′) is particularly preferable because the acid diffusion is quite small.

Further, as other acid generators, it is also possible to use sulfonium salts and iodonium salts having an anion having an aromatic ring substituted with an iodine atom, represented by the following formula (5-1) or (5-2).

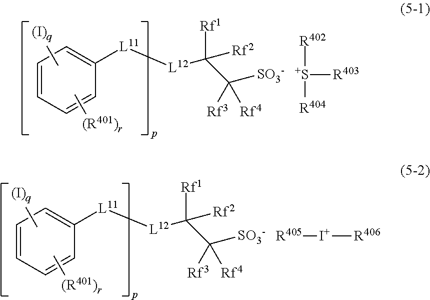

In the general formulae (5-1) and (5-2), “p” represents 1, 2, or 3, “q” and “r” represent integers satisfying 1≤q≤5, 0≤r≤3, and 1≤q+r≤5. “q” is preferably 1, 2, or 3, more preferably 2 or 3. “r” is preferably 0, 1, or 2.

In the formulae (5-1) and (5-2), L¹¹ represents a single bond, an ether bond, an ester bond, or a saturated hydrocarbylene group having 1 to 6 carbon atoms and optionally having an ether bond or an ester bond. The saturated hydrocarbylene group may be linear, branched, or cyclic.

In the formulae (5-1) and (5-2), Liz represents a single bond or a divalent linking group having 1 to 20 carbon atoms when “p” represents 1, and Liz represents a (p+1)-valent linking group having 1 to 20 carbon atoms when “p” represents 2 or 3. The linking group may have an oxygen atom, a sulfur atom, or a nitrogen atom.

In the formulae (5-1) and (5-2), R^{4′} represents a hydroxy group, a carboxy group, a fluorine atom, a chlorine atom, a bromine atom, an amino group, a hydrocarbyl group having 1 to 20 carbon atoms, a hydrocarbyloxy group having 1 to 20 carbon atoms, a hydrocarbylcarbonyl group having 2 to 20 carbon atoms, a hydrocarbyloxycarbonyl group having 2 to 20 carbon atoms, a hydrocarbylcarbonyloxy group having 2 to 20 carbon atoms, a hydrocarbylsulfonyloxy group having 1 to 20 carbon atoms, “—N(R^{401A})(R^{401B})”, “—N(R^{401C})—C(═O)—R^{401D}”, or “—N(R^{401C}) —C(═O)—O—R^{401D}”. The hydrocarbyl group, the hydrocarbyloxy group, the hydrocarbylcarbonyl group, the hydrocarbyloxycarbonyl group, the hydrocarbylcarbonyloxy group, and the hydrocarbylsulfonyloxy group may have a fluorine atom, a chlorine atom, a bromine atom, a hydroxy group, an amino group, or an ether bond. R^{401A} and R^{401B} each independently represent a hydrogen atom or a saturated hydrocarbyl group having 1 to 6 carbon atoms. R^{401C} represents a hydrogen atom or a saturated hydrocarbyl group having 1 to 6 carbon atoms and may have a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. R^{401D} represents an aliphatic hydrocarbyl group having 1 to 16 carbon atoms, an aryl group having 6 to 14 carbon atoms, or an aralkyl group having 7 to 15 carbon atoms, and may have a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. The aliphatic hydrocarbyl group may be a saturated or unsaturated group, and may be linear, branched, or cyclic. The hydrocarbyl group, the hydrocarbyloxy group, the hydrocarbylcarbonyl group, the hydrcarbyloxycarbonyl group, the hydrocarbylcarbonyloxy group, and the hydrocarbylsulfonyloxy group may be linear, branched, or cyclic groups. When “p” and/or “r” represent 2 or more, each R⁴⁰¹ may be the same as or different from each other.

Among these, R⁴⁰1 preferably represents a hydroxy group, “—N(R^{401C})—C(═O) —R^{401D}”, “—N(R^{401C})—C(═O)—O—R^{401D}”, a fluorine atom, a chlorine atom, a bromine atom, a methyl group, a methoxy group, or the like.

In the formulae (5-1) and (5-2), Rf¹ to Rf⁴ each independently represent a hydrogen atom, a fluorine atom, or a trifluoromethyl group, and at least one of them represents a fluorine atom or a trifluoromethyl group. Rf¹ and Rf² may be integrated to form a carbonyl group. In particular, both of Rf³ and Rf⁴ preferably represent fluorine atoms.

In the general formulae (5-1) and (5-2), R⁴⁰² to R⁴⁰⁶ are each independently a halogen atom or a hydrocarbyl group having 1 to 20 carbon atoms and optionally having a halogen atom. The hydrocarbyl group may be saturated or unsaturated, and may be linear, branched, or cyclic. In addition, a part or all of the hydrogen atoms of the hydrocarbyl group may be substituted with a hydroxy group, a carboxy group, a halogen atom, a cyano group, a nitro group, a mercapto group, a sultone ring, a sulfo group, or a sulfonium salt-containing group, and a part of the “—CH₂—” of the hydrocarbyl group may be substituted with an ether bond, an ester bond, a carbonyl group, an amide bond, a carbonate bond, or a sulfonate ester bond. Further, R⁴⁰² and R⁴⁰³ may be bonded to each other to form a ring together with the sulfur atom to which they are bonded.

Specific examples of the cation of the sulfonium salt represented by the formula (5-1) include the same as those exemplified as the sulfonium cation in the repeating units h2 to h4 described above. Specific examples of the cation of the iodonium salt represented by the formula (5-2) include the same as those exemplified as the diphenyliodonium cation in the component C-2 described above.

Specific examples of the anion of the onium salt represented by the formula (5-1) or (5-2) include those shown below, but are not limited thereto.

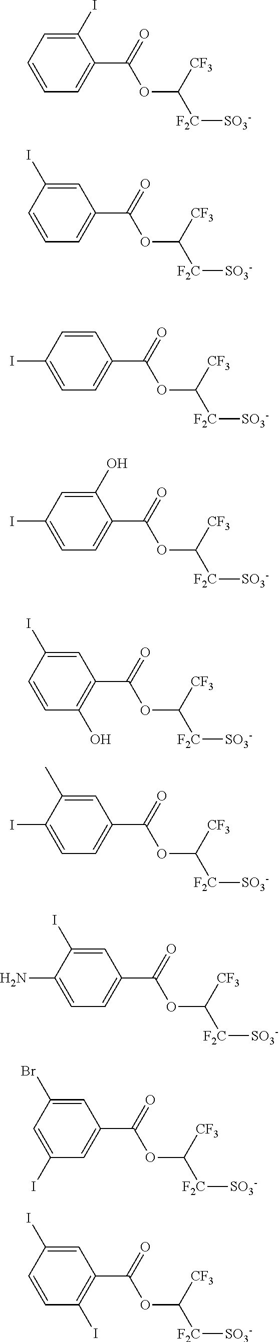

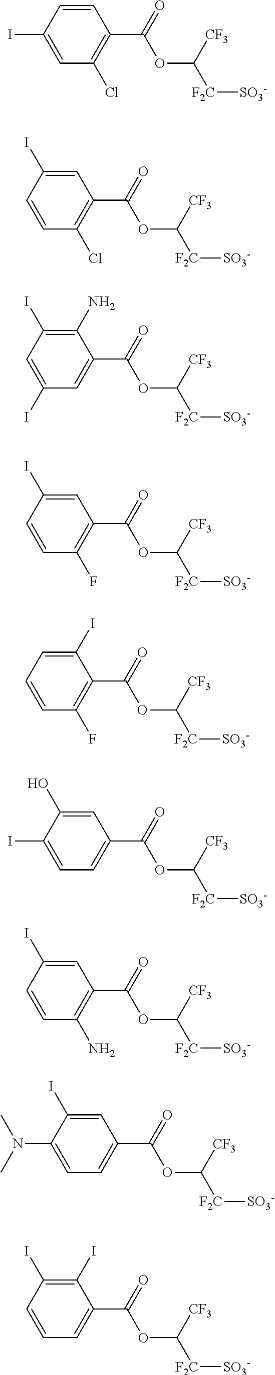

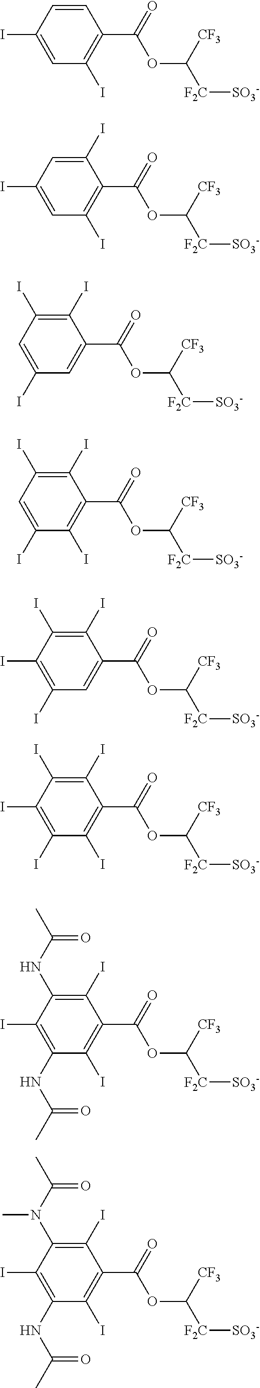

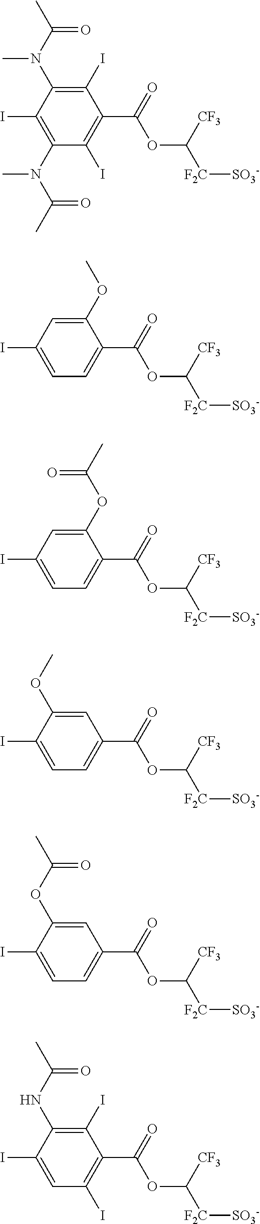

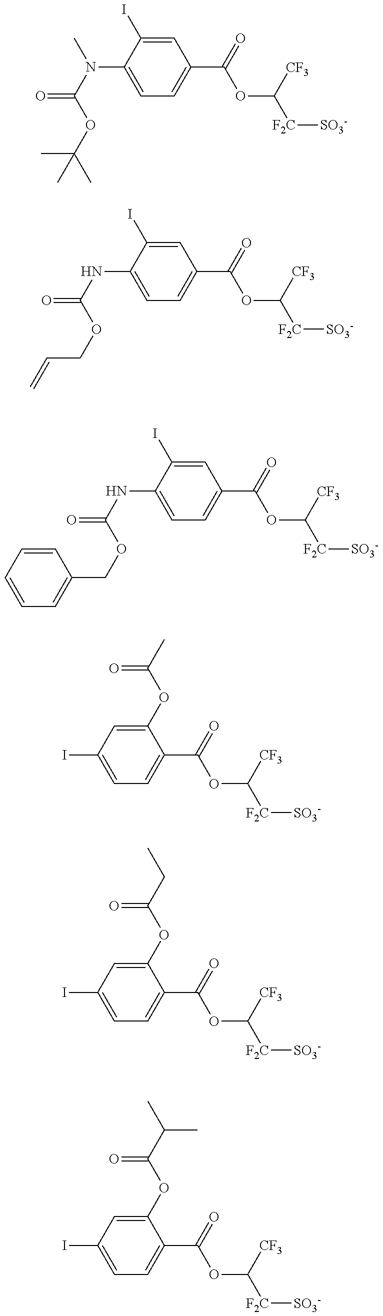

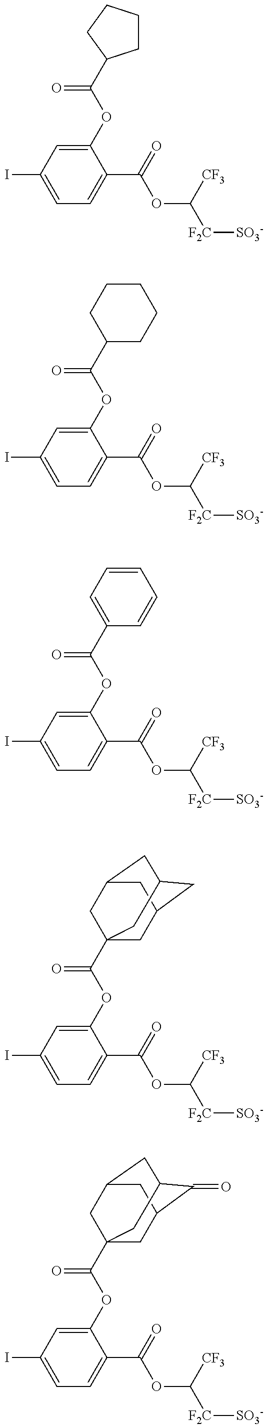

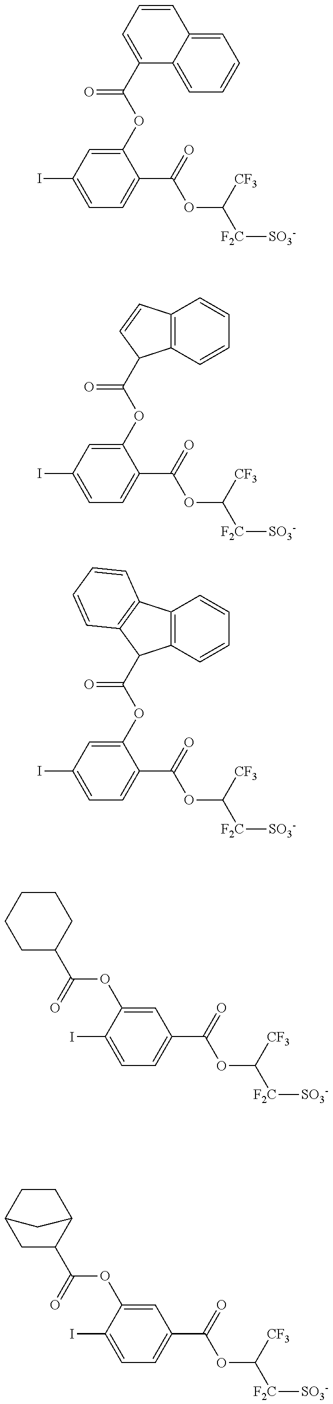

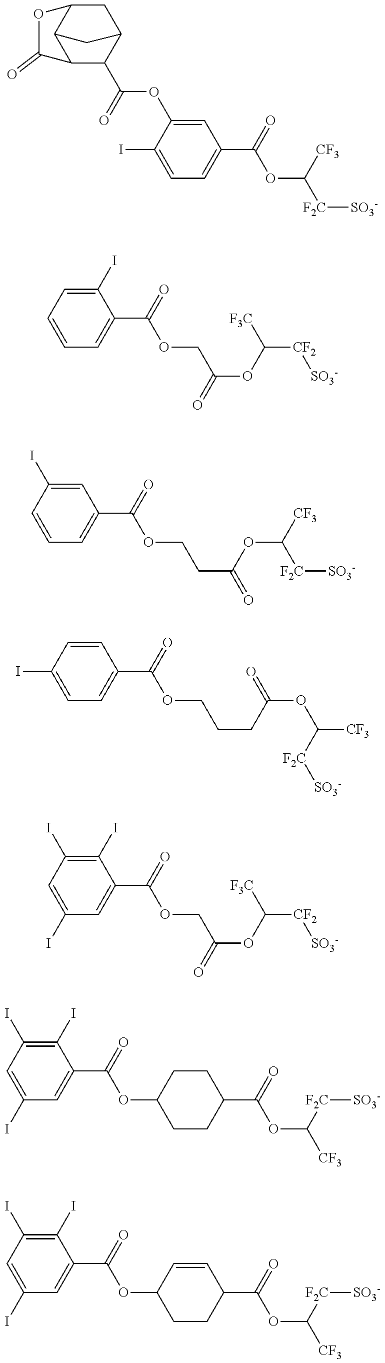

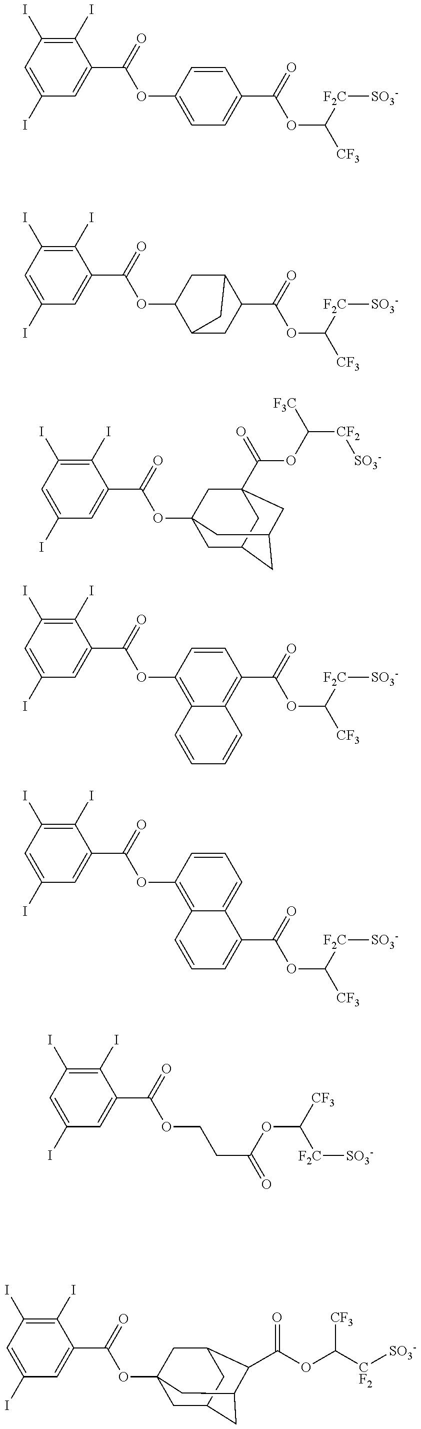

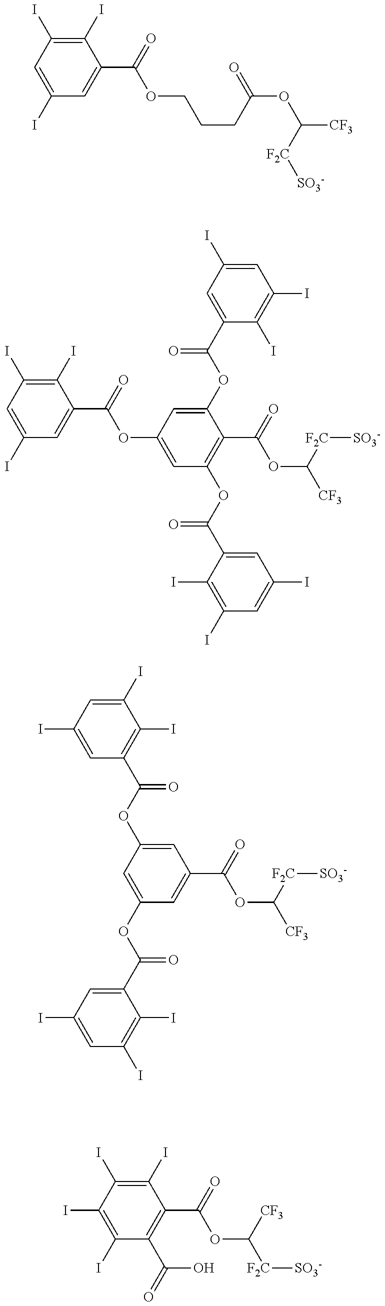

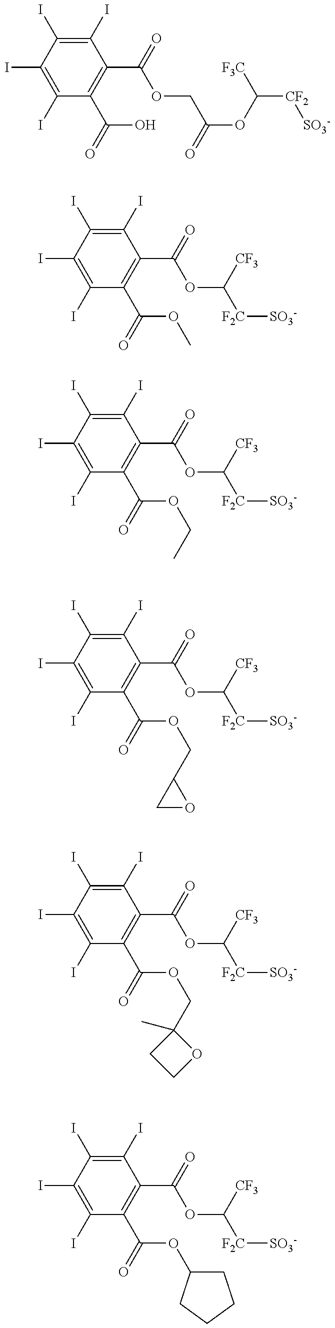

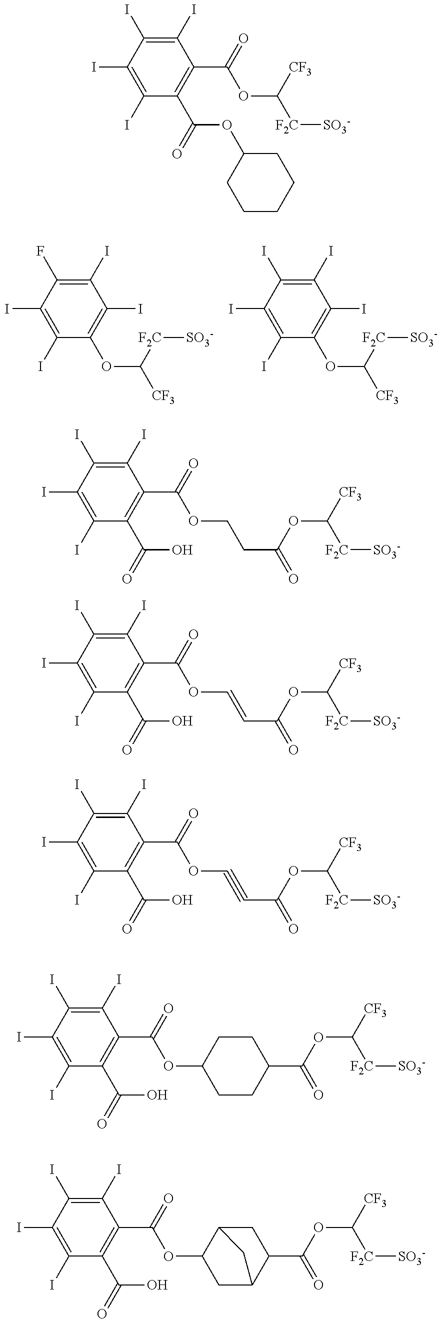

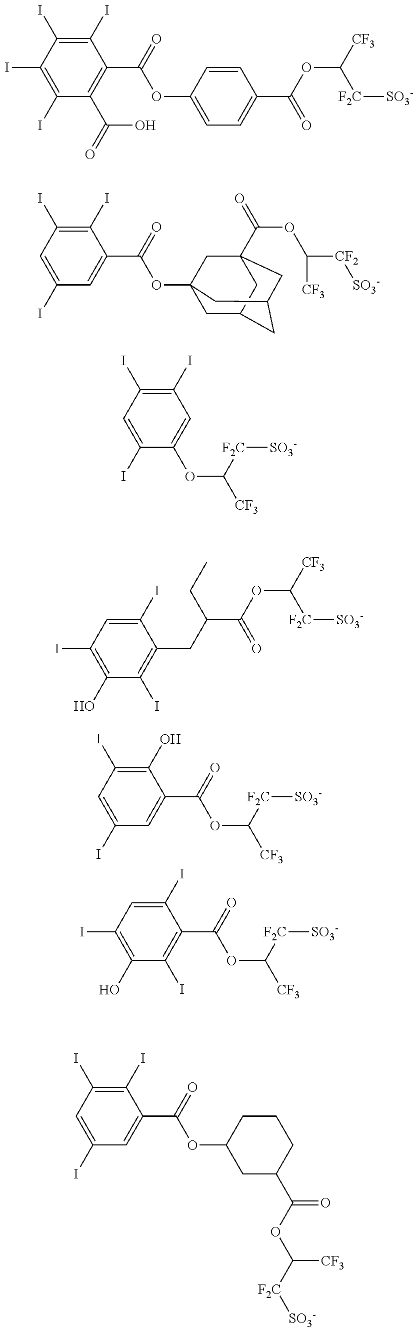

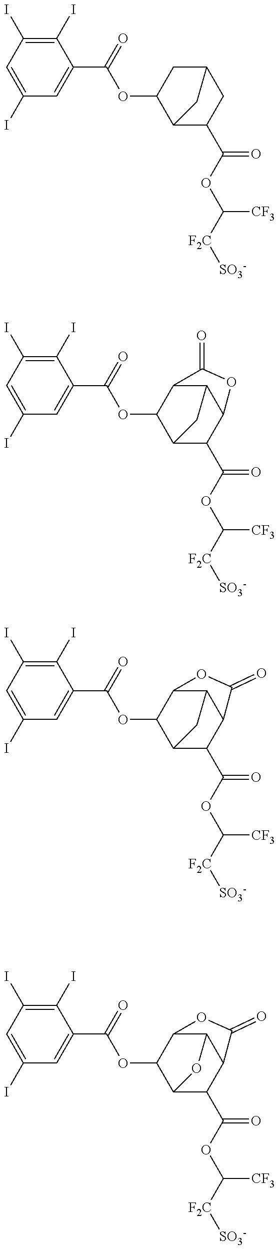

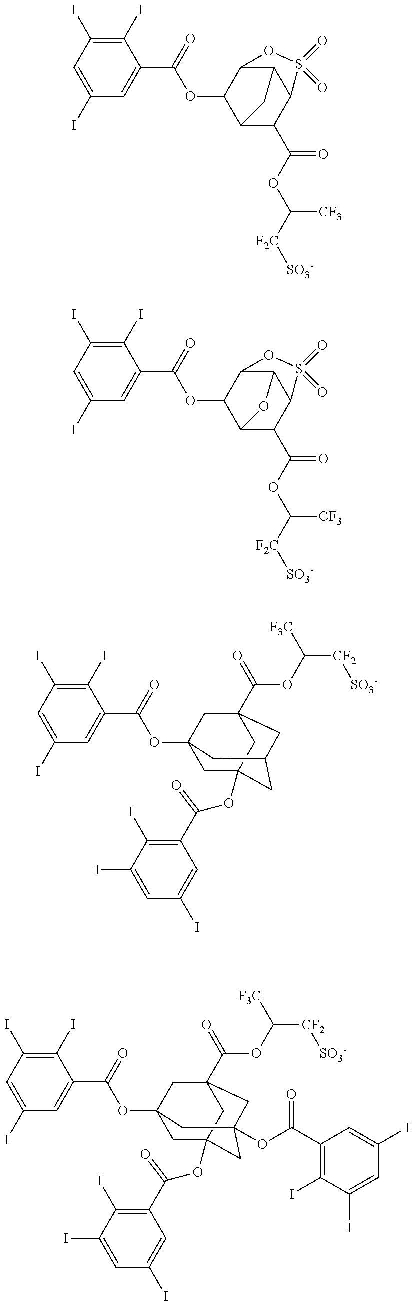

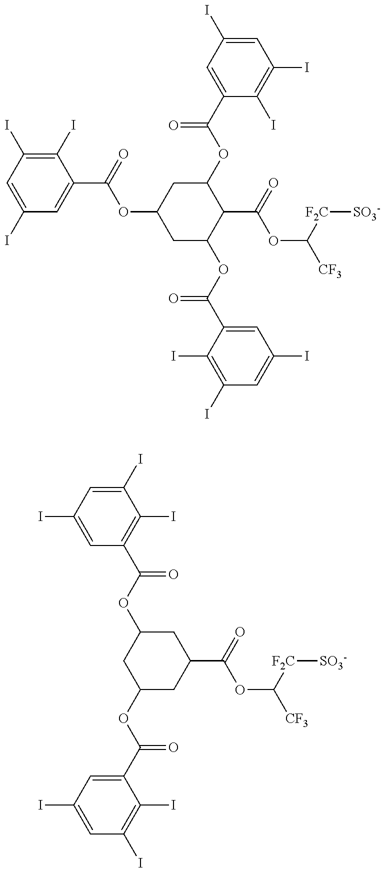

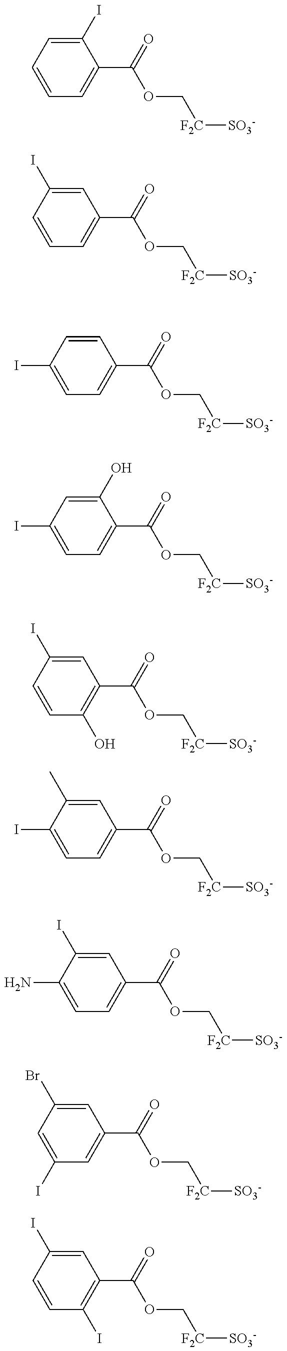

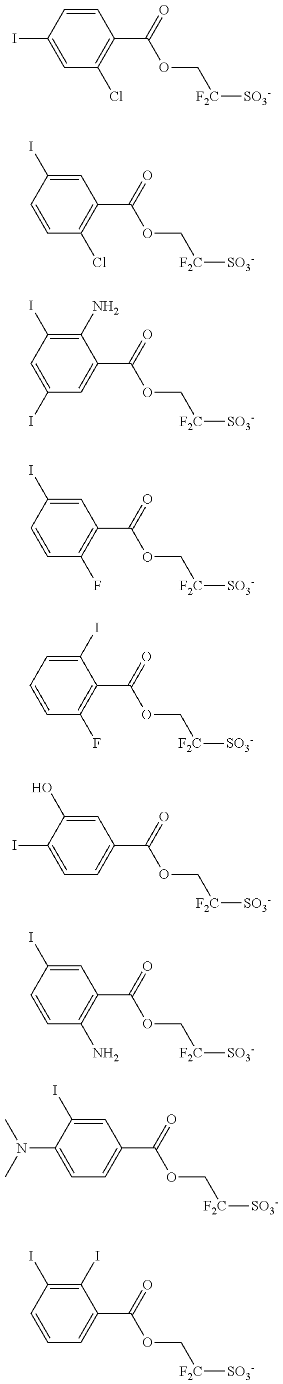

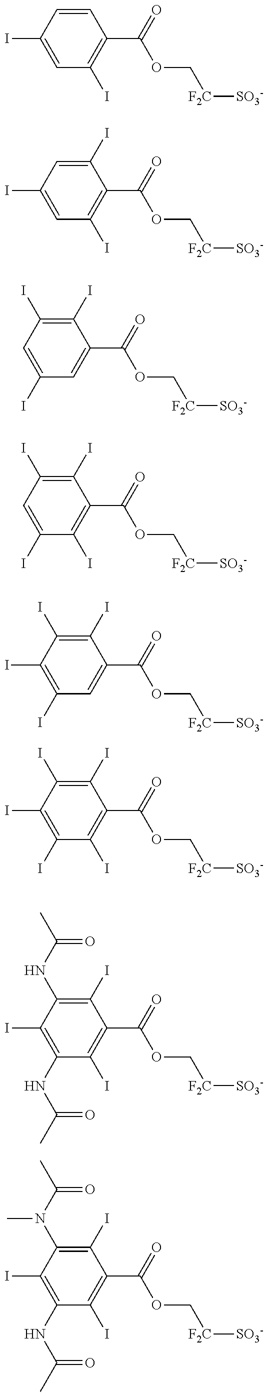

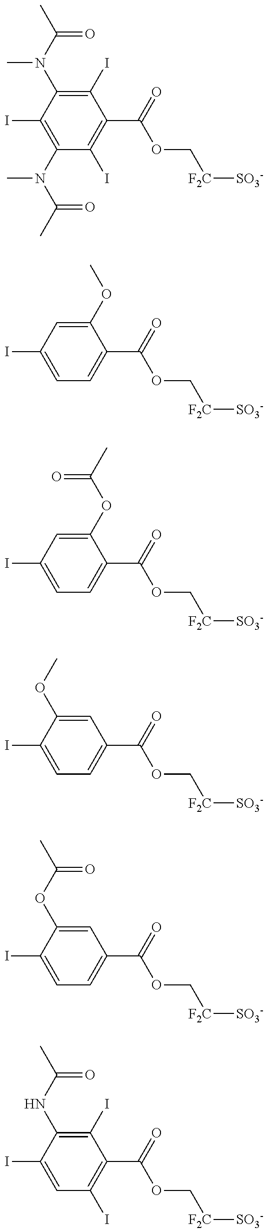

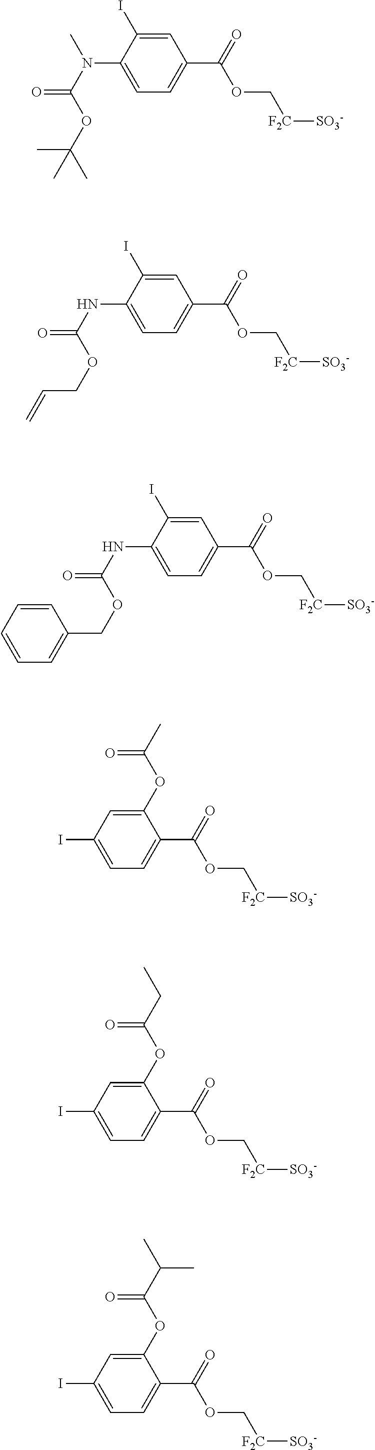

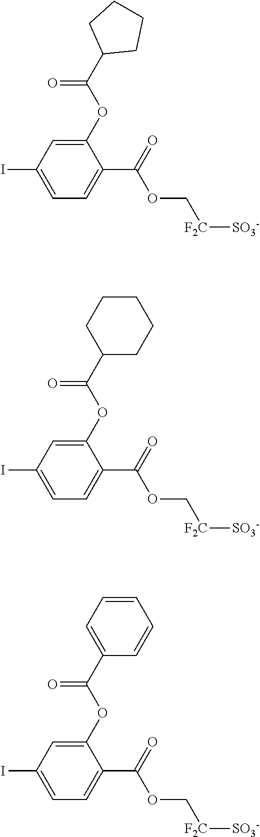

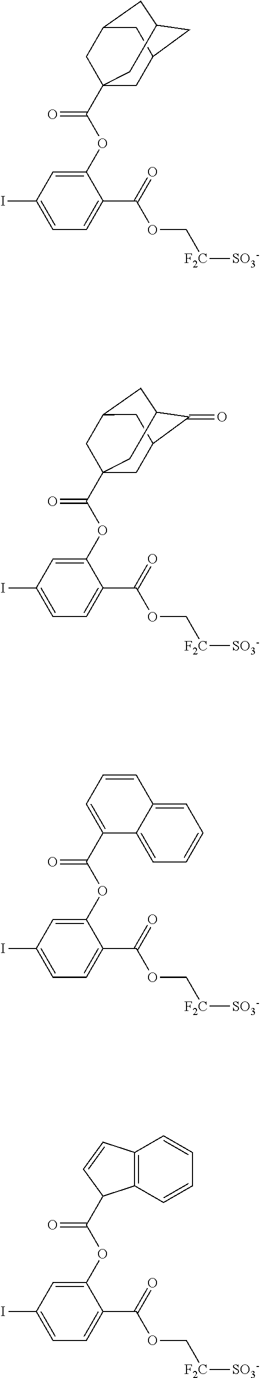

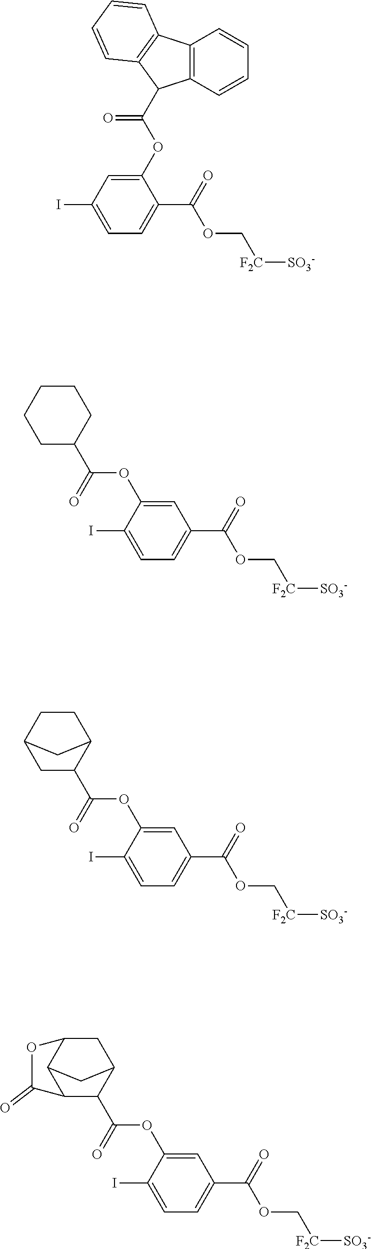

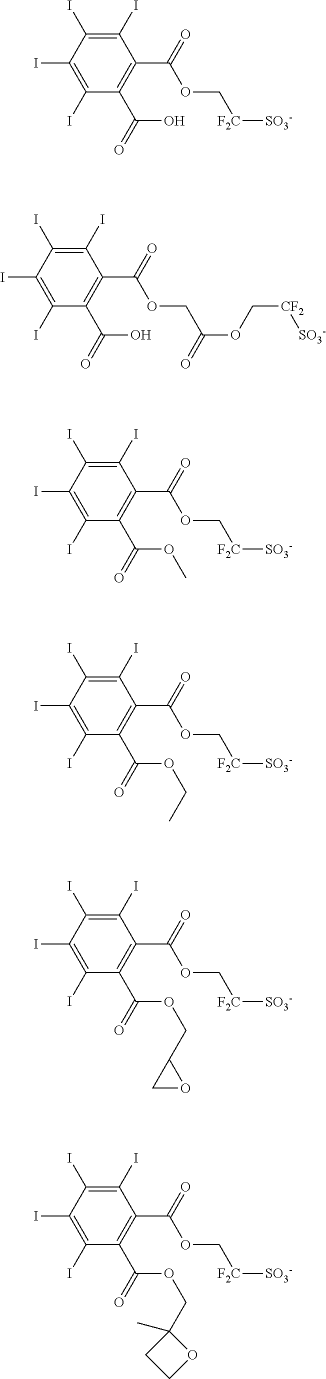

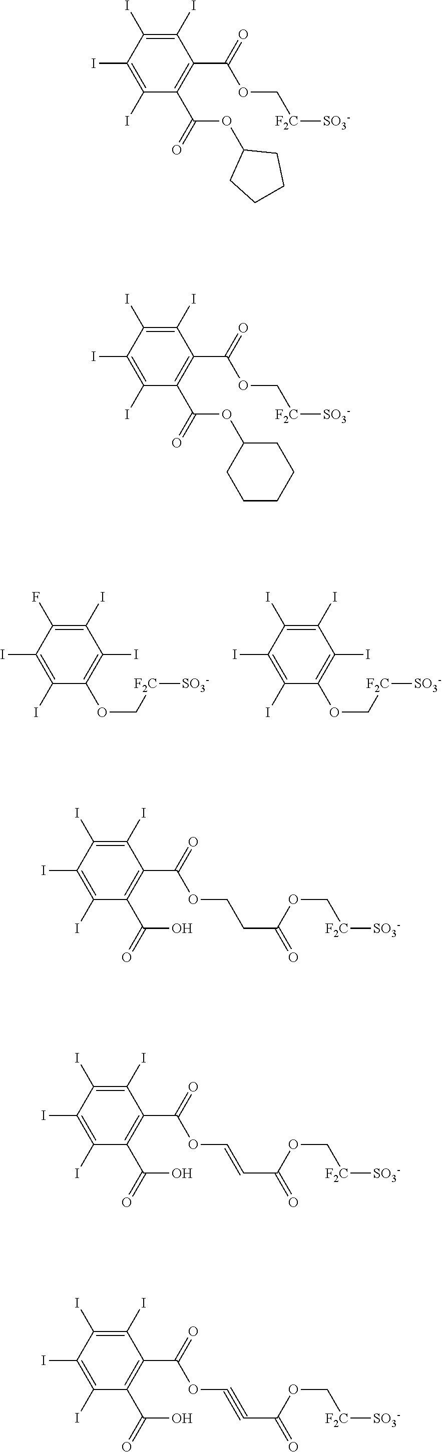

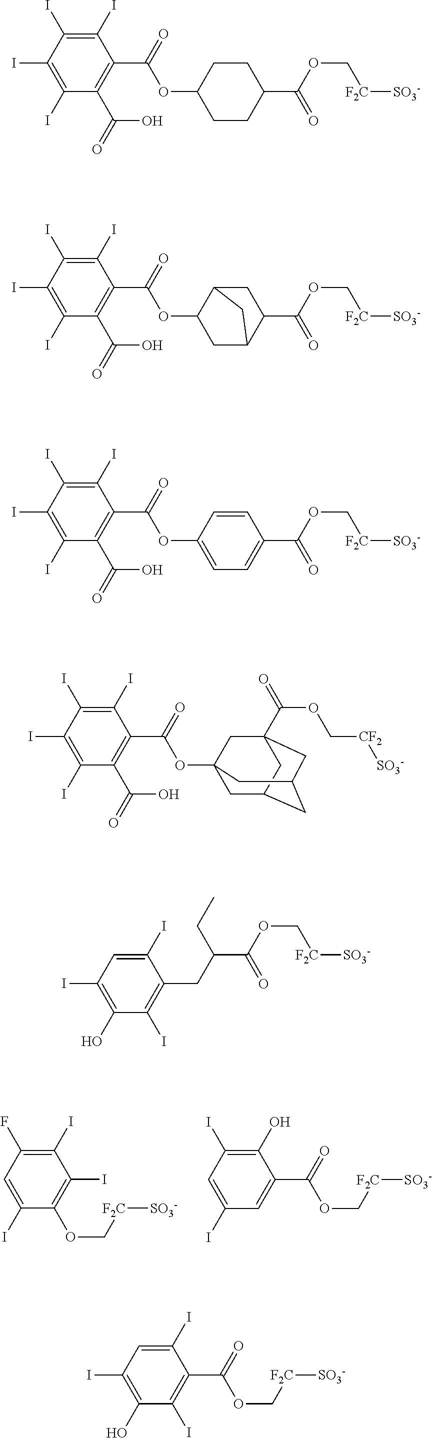

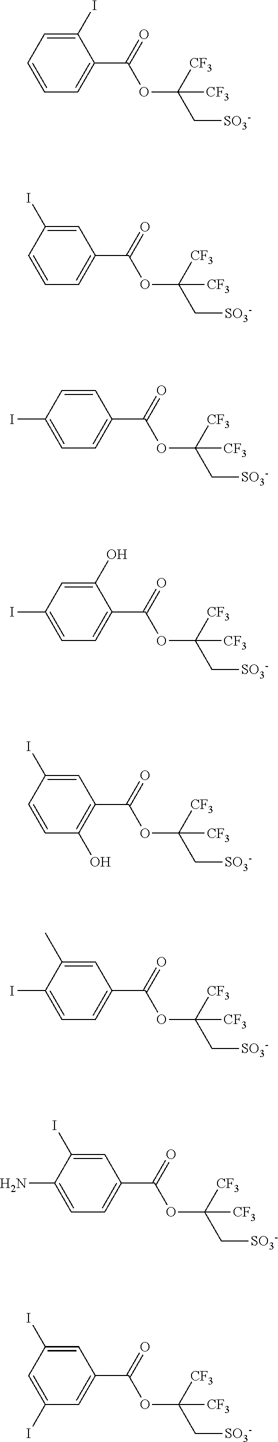

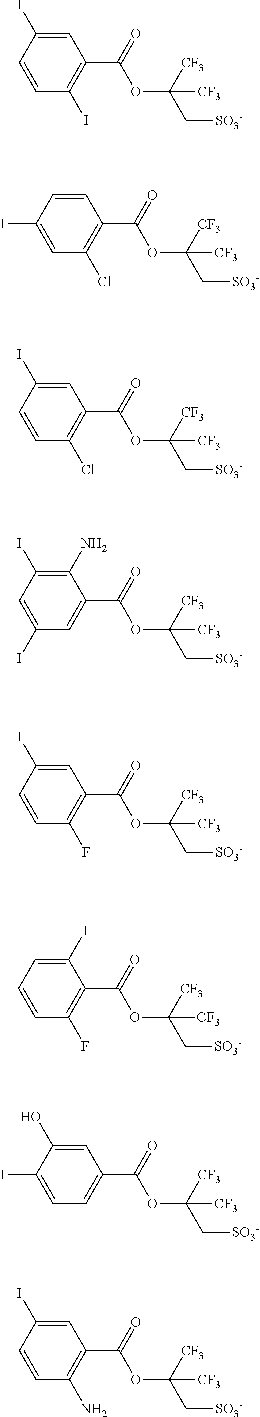

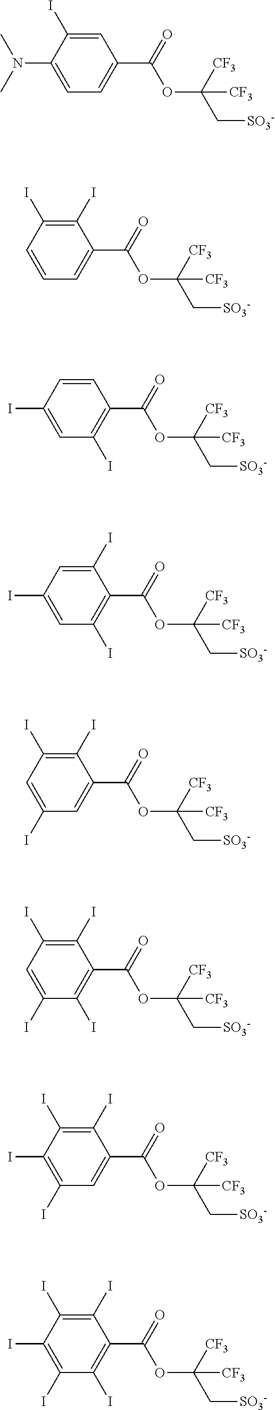

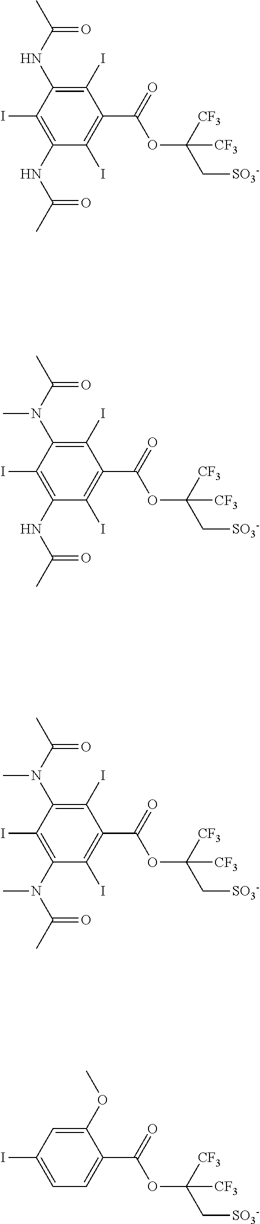

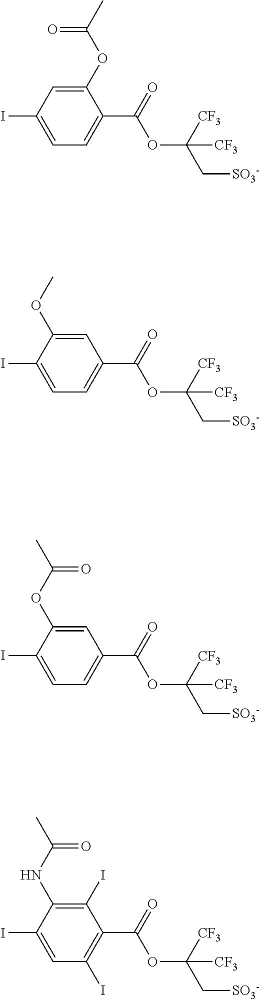

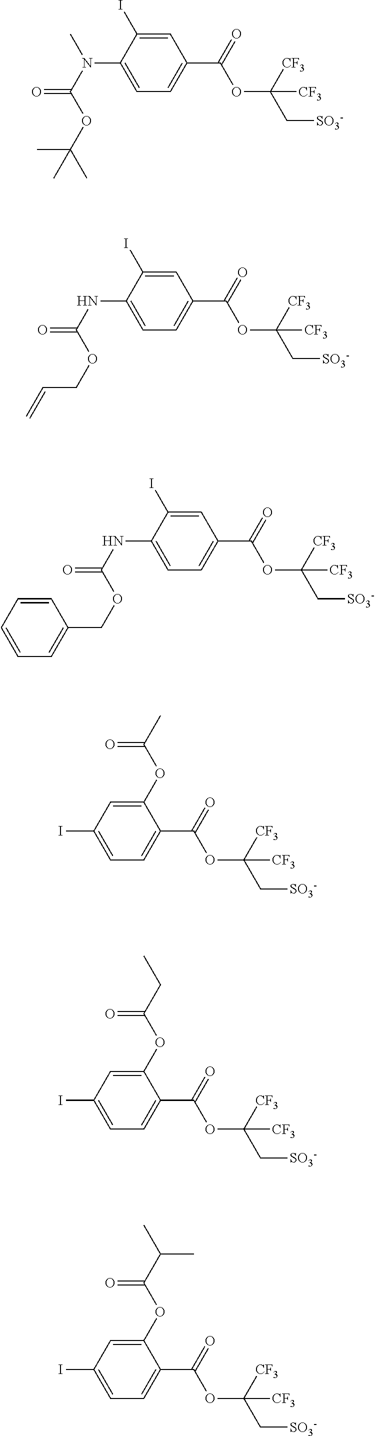

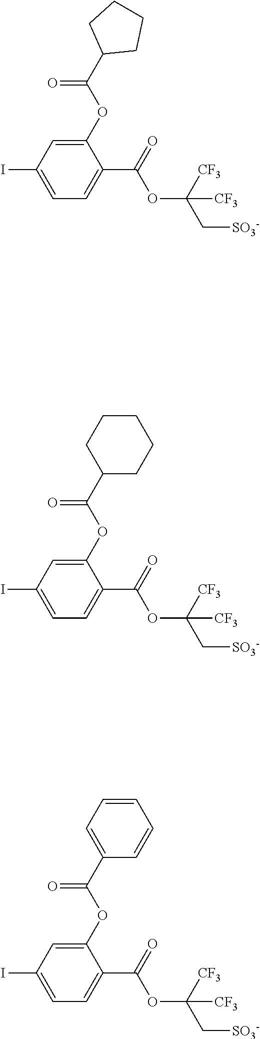

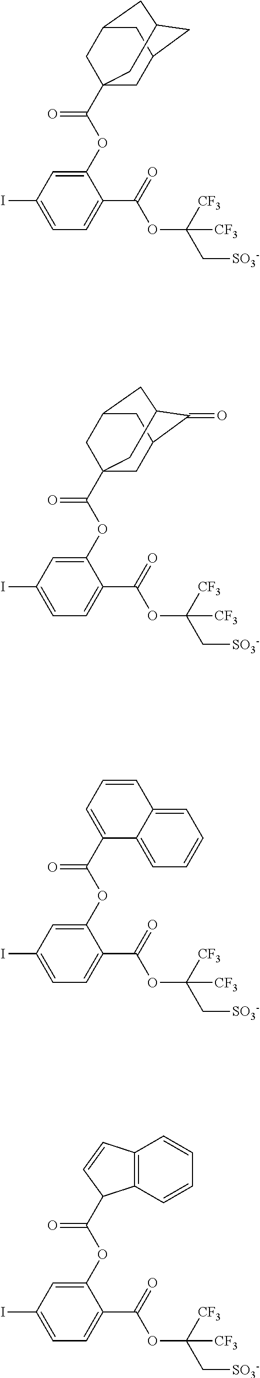

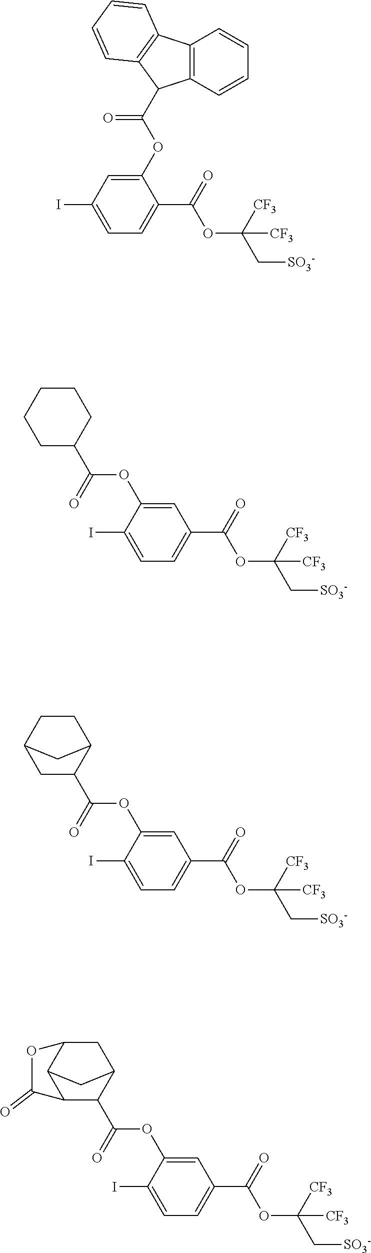

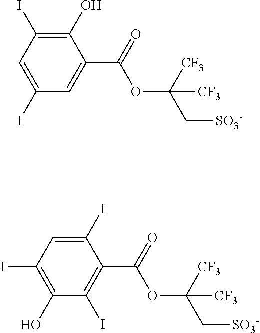

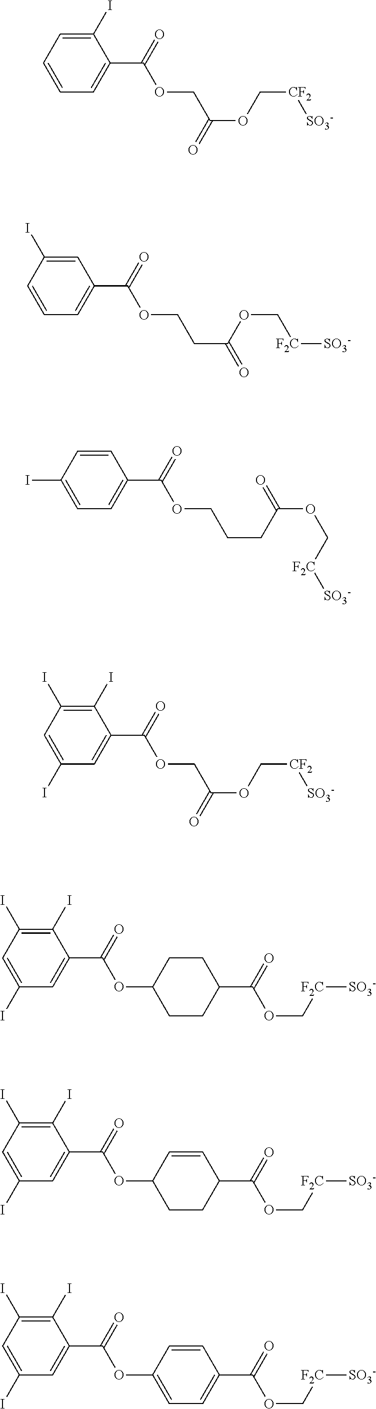

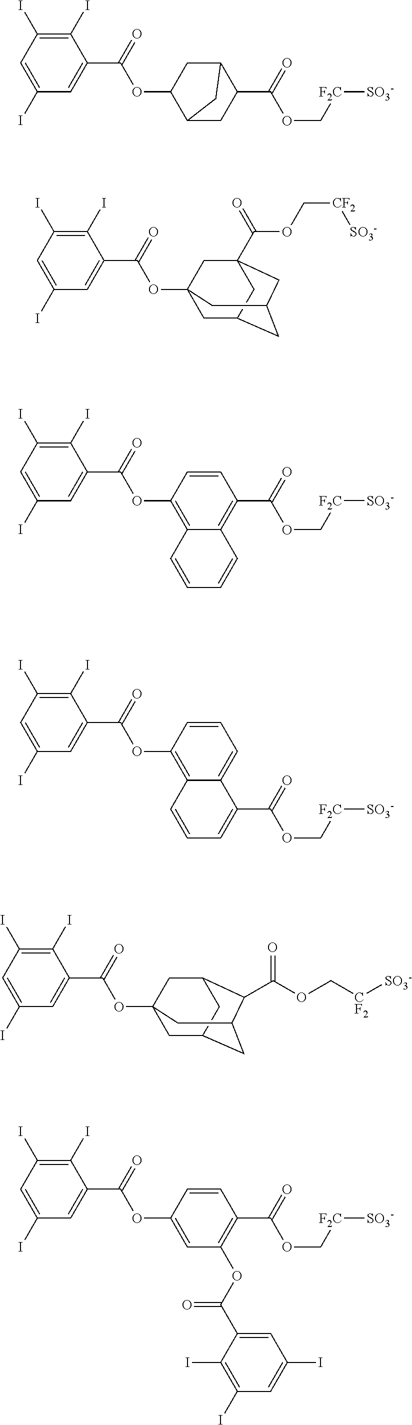

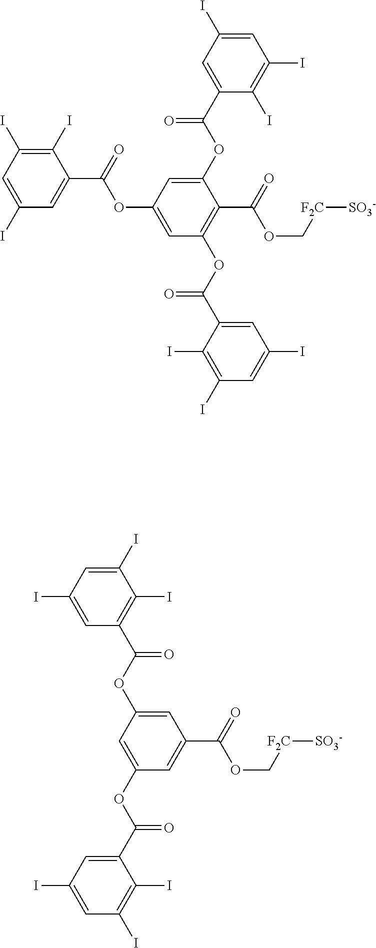

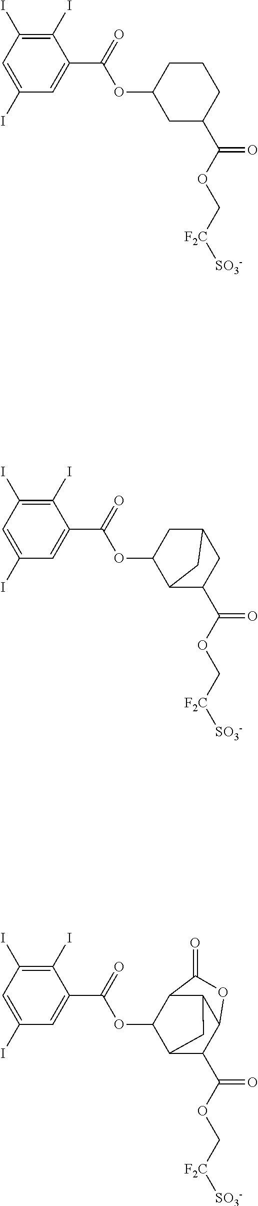

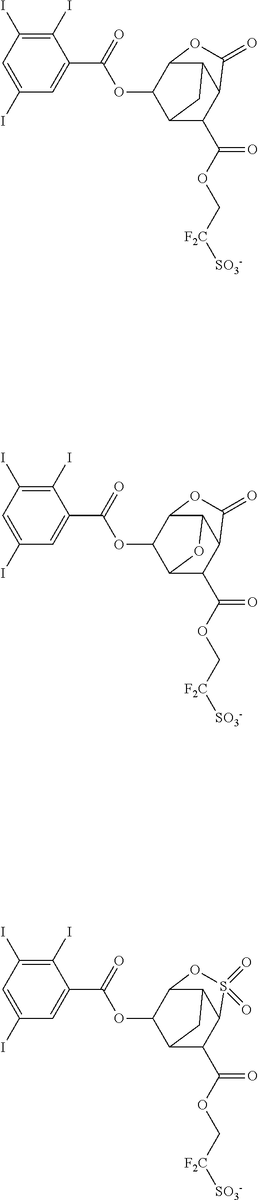

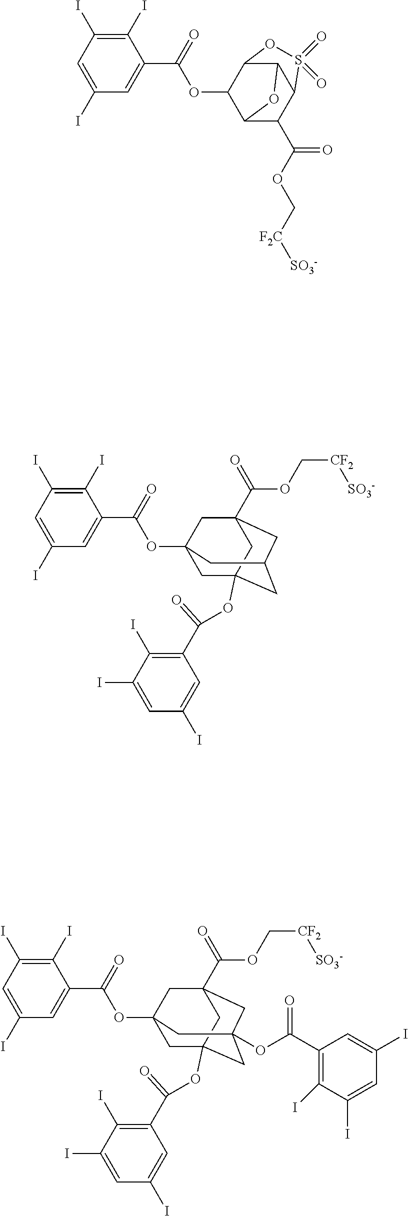

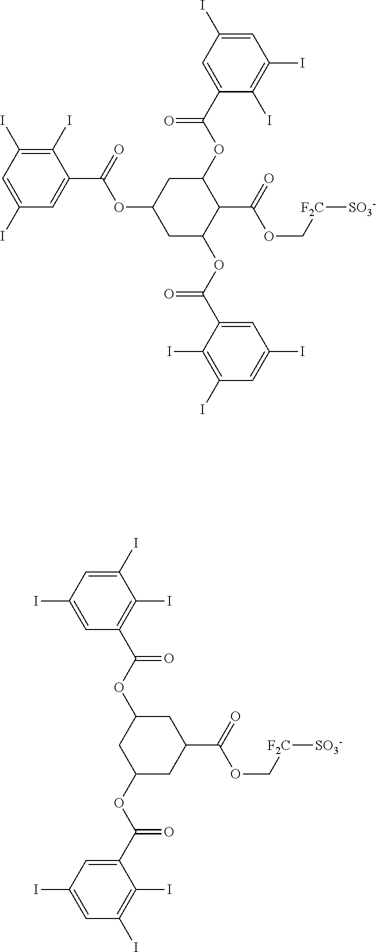

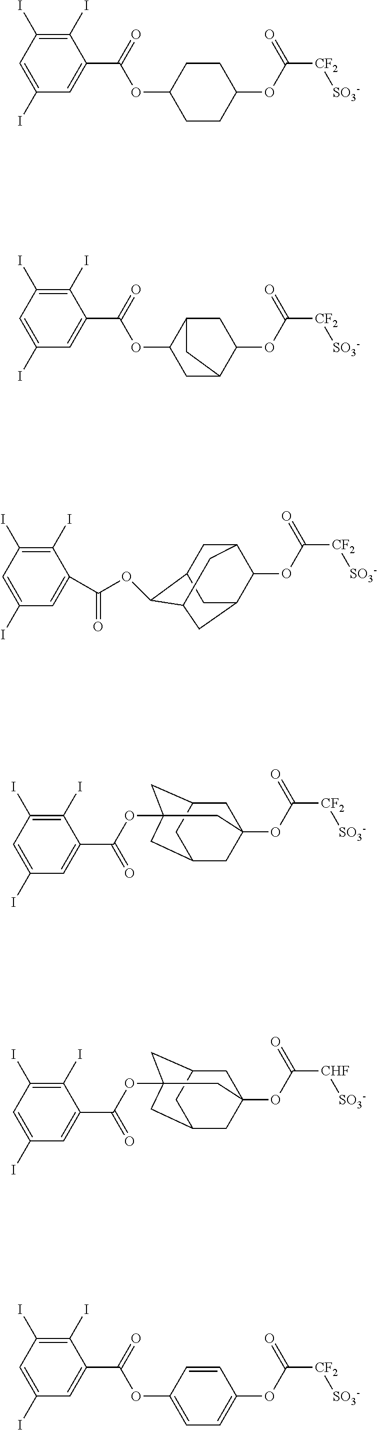

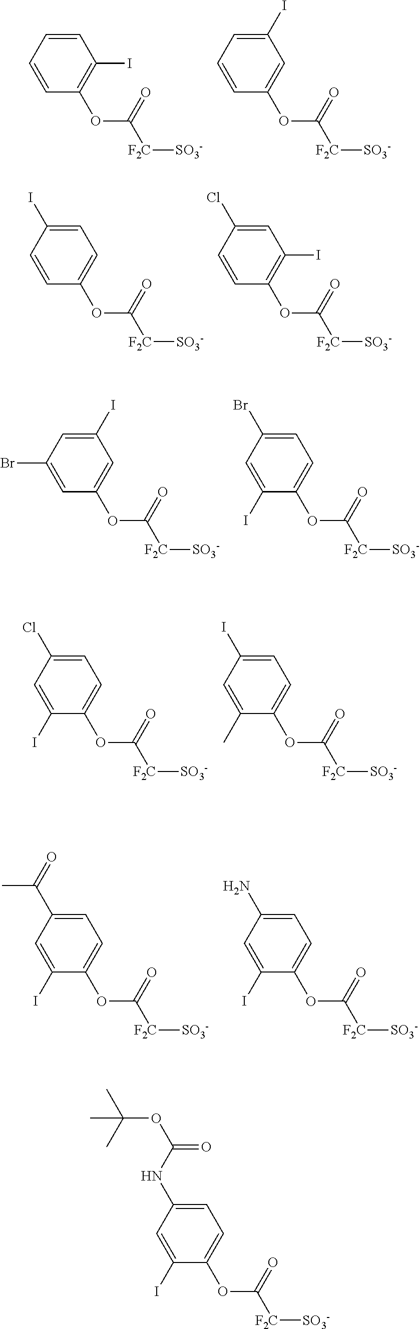

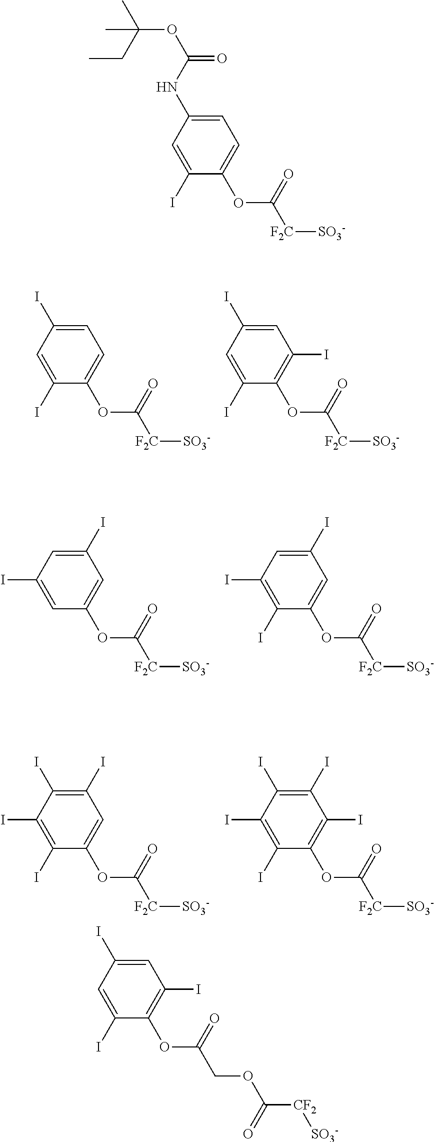

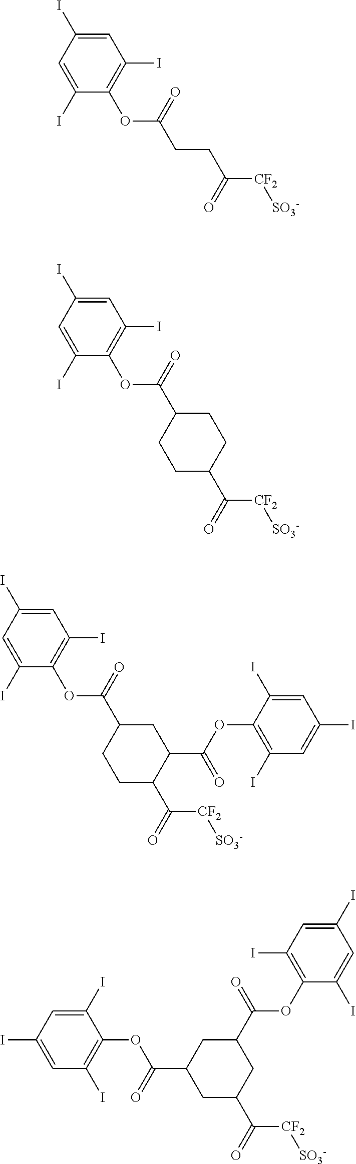

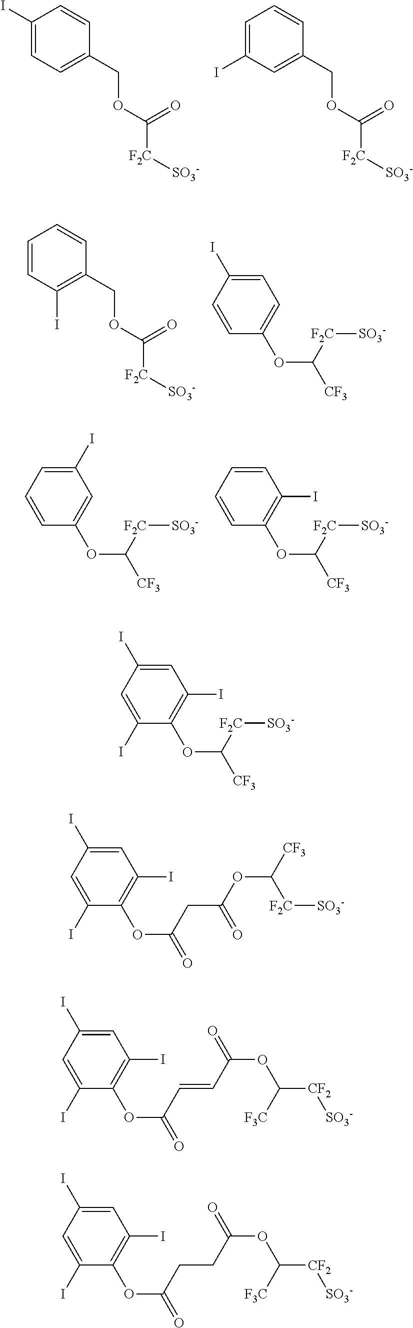

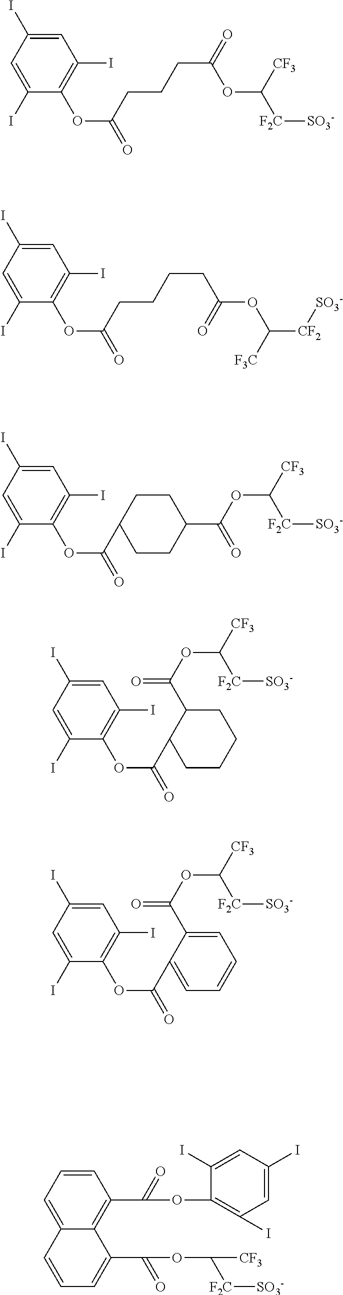

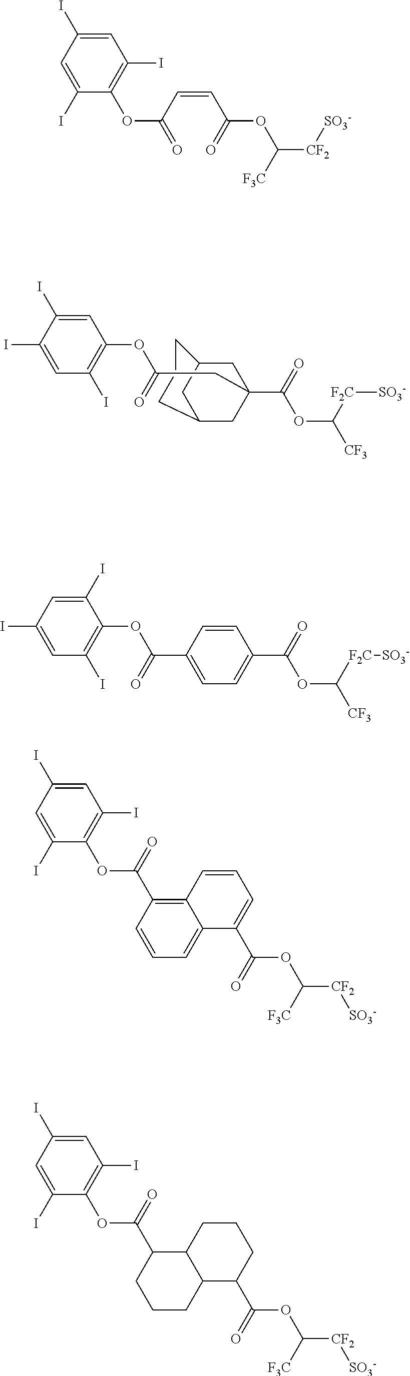

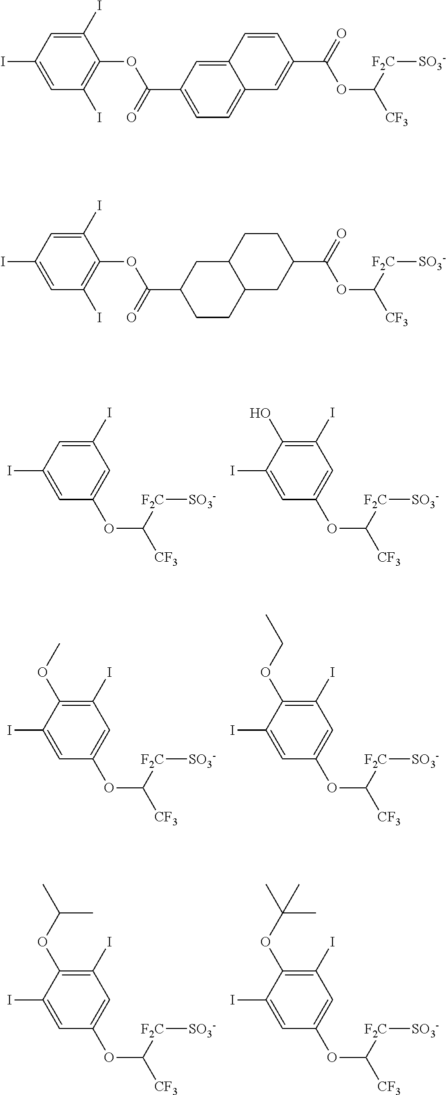

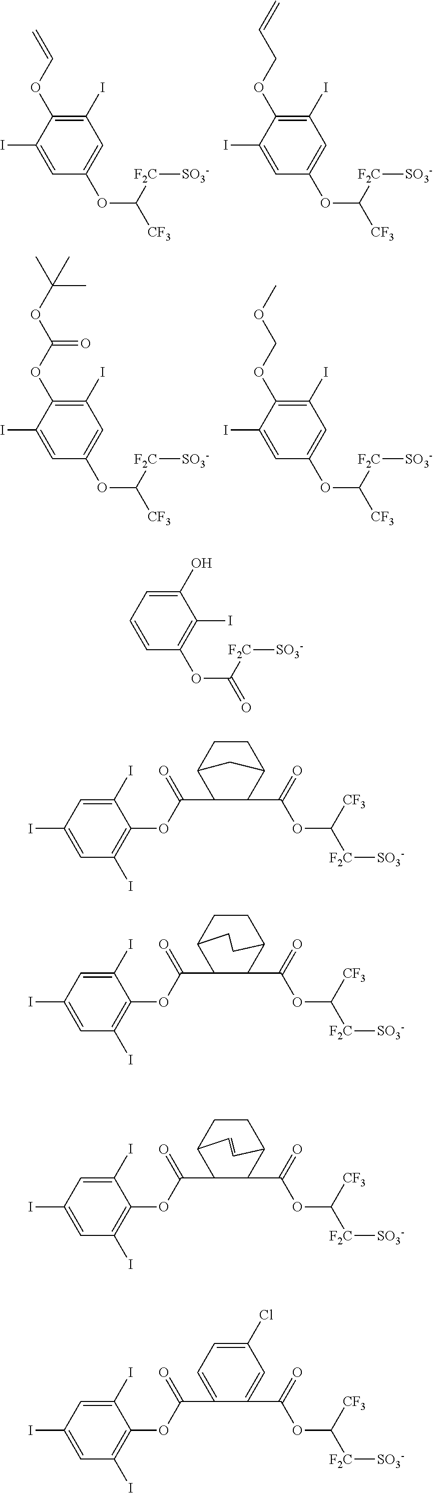

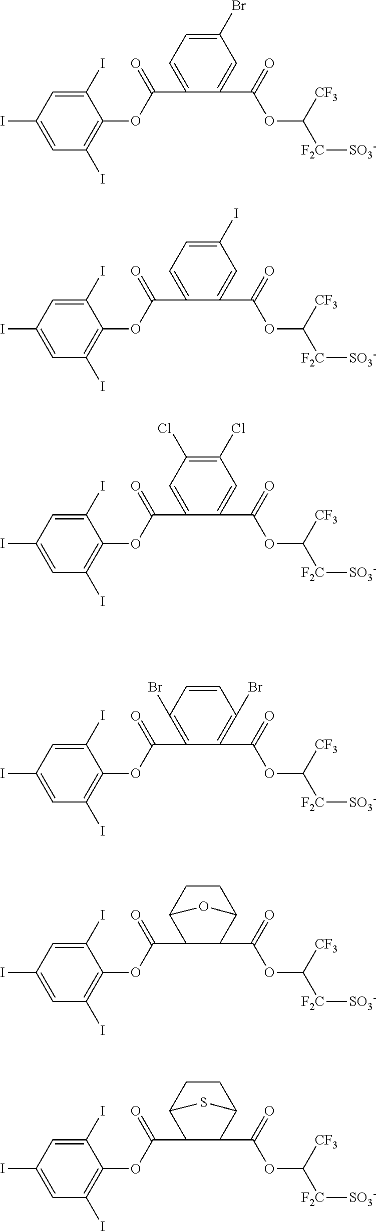

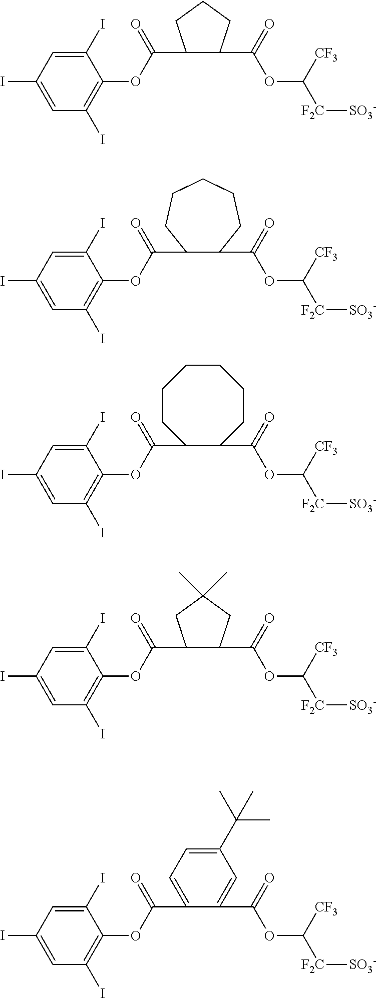

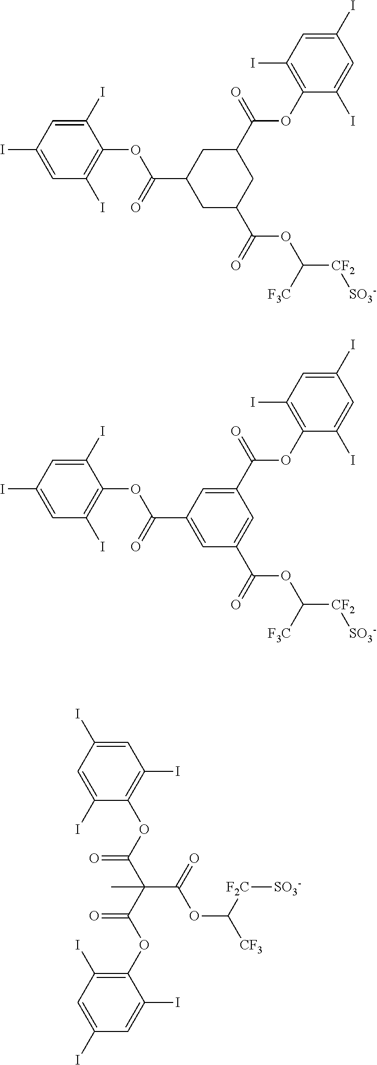

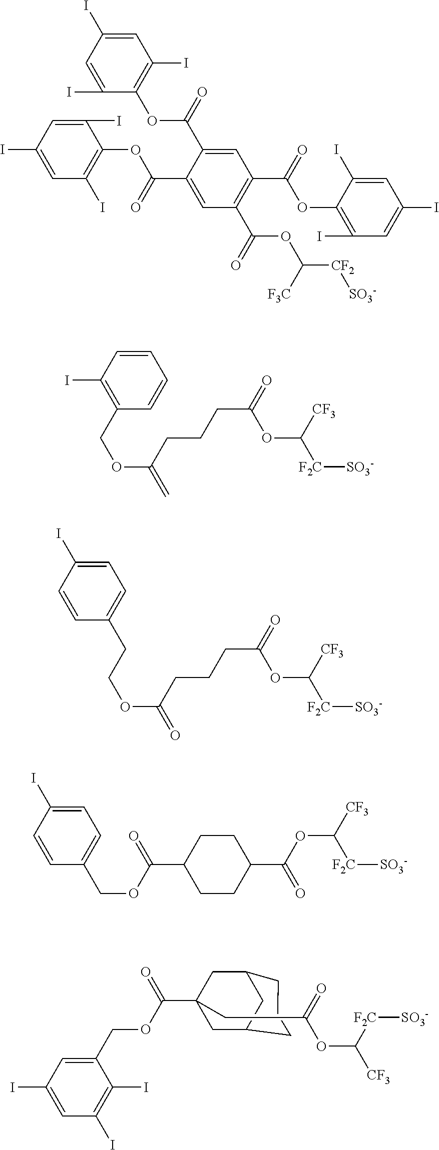

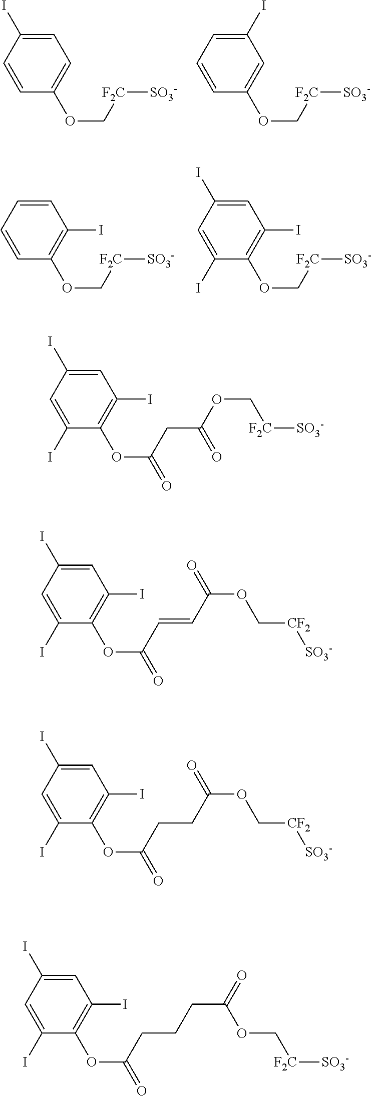

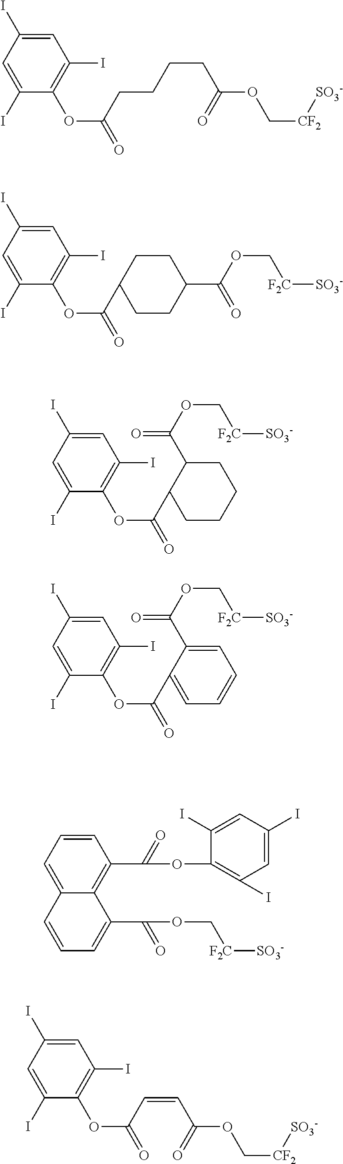

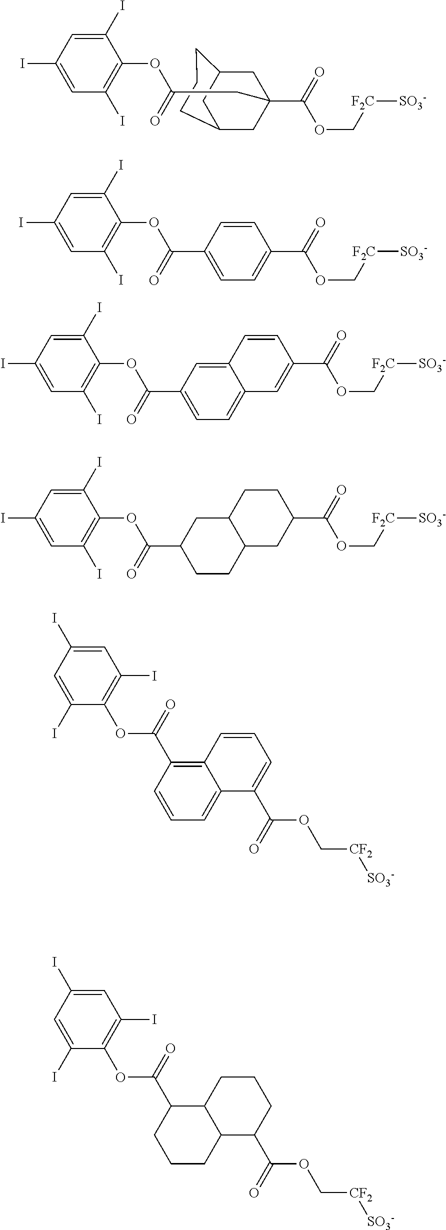

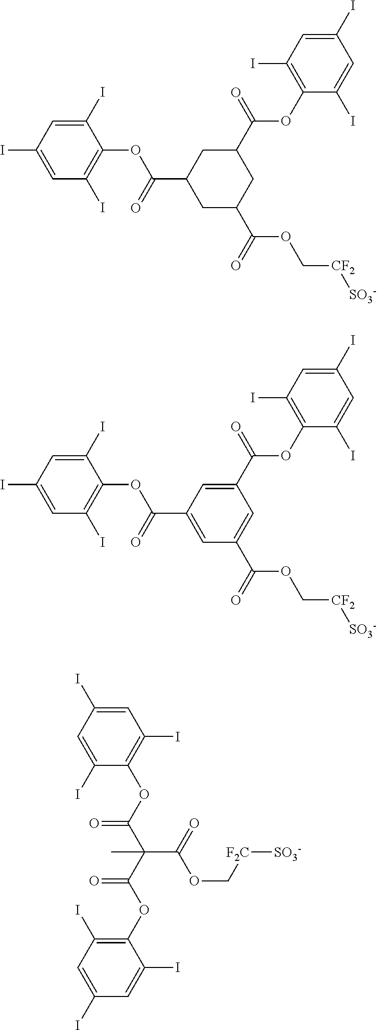

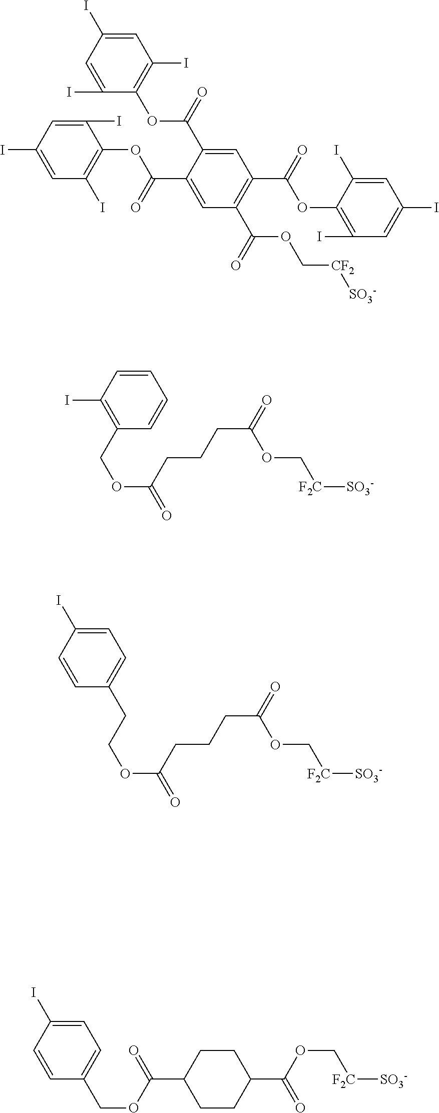

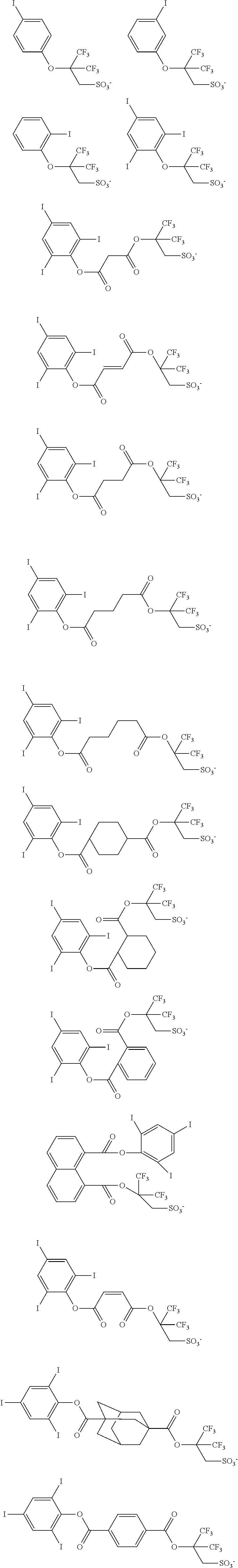

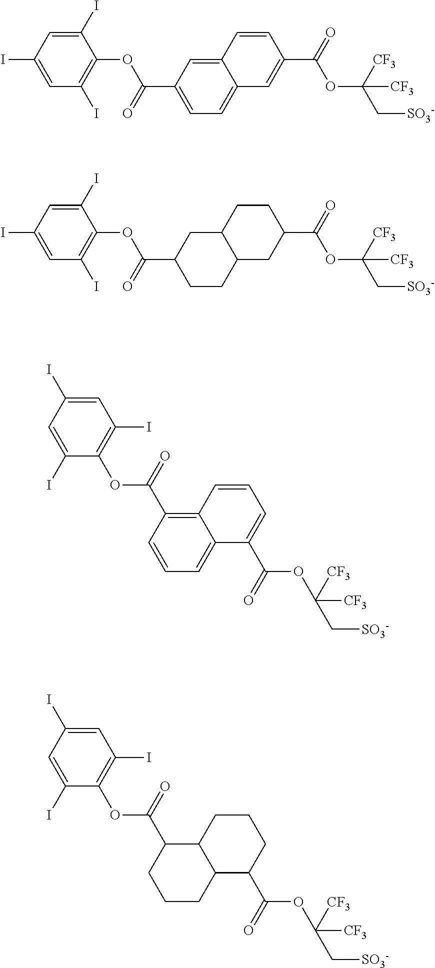

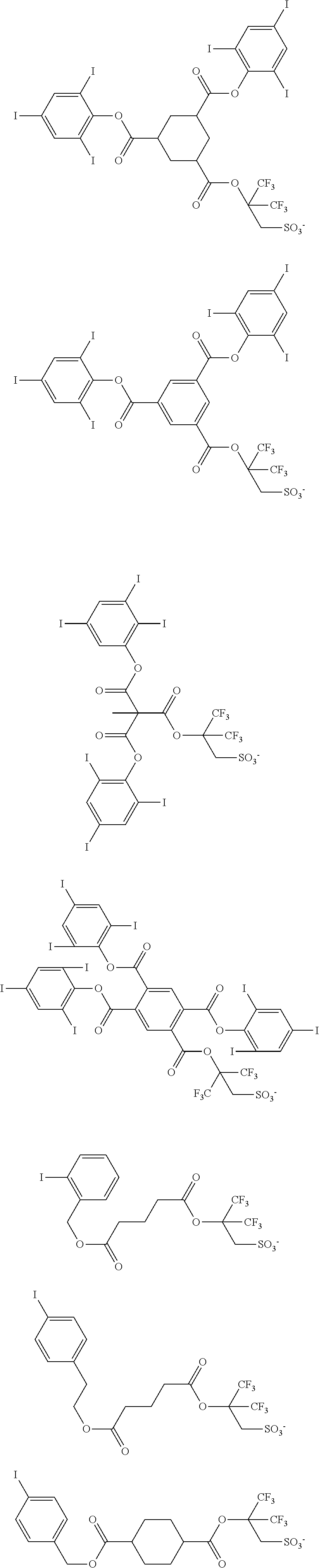

When the photo-acid generator of the component (B) is contained in the inventive chemically-amplified resist composition, the content of the photo-acid generator is preferably 0.1 to 40 parts by mass, and more preferably 0.5 to 30 parts by mass, relative to 80 parts by mass of the base polymer (component A) or (component A-1). When the addition amount of the photo-acid generator of the component (B) is within the above range, it is preferable because of good resolution and no risk of causing a foreign matter problem after the development or during the removal of the resist film. One kind of the photo-acid generators of the component (B) may be used, or two or more kinds thereof may be used in combination.

### [(E) Surfactant]

The inventive chemically-amplified resist composition may further contain a surfactant as the component (E). The preferable component (E) surfactant is: insoluble or hardly soluble in water and soluble in alkaline developer; or insoluble or hardly soluble in water and alkaline developer. As such a surfactant, surfactants described in JP2010-215608A and JP2011-16746A can be referred.

Among the surfactants described in the above patent publications, the surfactant insoluble or hardly soluble in water and an alkaline developer is preferably FC-4430 (manufactured by 3M Company), SURFLON (registered trademark)S-381 (manufactured by AGC Seimi Chemical Co., Ltd.), OLFIN (registered trademark) E1004 (manufactured by Nissin Chemical Industry Co., Ltd.), KH-20 and KH-30 (manufactured by AGC Seimi Chemical Co., Ltd.), an oxetane ring-opening polymerized product represented by the following formula (surf-1), or the like.

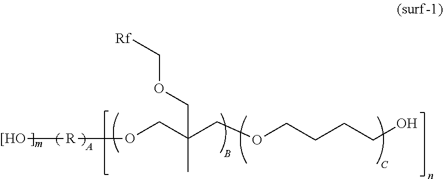

Here, “R”, Rf, “A”, “B”, “C”, “m”, and “n” are applied only in the formula (surf-1) regardless of the above description. “R” represents a divalent to tetravalent aliphatic group having 2 to 5 carbon atoms. Examples of the divalent aliphatic group include an ethylene group, a 1,4-butylene group, a 1,2-propylene group, a 2,2-dimethyl-1,3-propylene group, and a 1,5-pentylene group. Examples of the trivalent or tetravalent aliphatic group include the following groups.

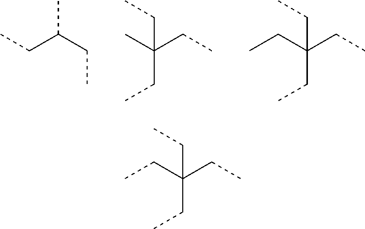

In the formula, a broken line represents a bond. The groups each are a partial structure derived from glycerol, trimethylolethane, trimethylolpropane, and pentaerythritol, respectively.

Among these, the preferable groups are a 1,4-butylene group, a 2,2-dimethyl-1,3-propylene group, etc.

Rf represents a trifluoromethyl group or a pentafluoroethyl group, and preferably a trifluoromethyl group. “m” represents an integer of 0 to 3. “n” represents an integer of 1 to 4. The sum of “n” and “m”, which represents a valency of “R”, represents an integer of 2 to 4. “A” represents 1. “B” represents an integer of 2 to 25, and preferably represents an integer of 4 to 20. “C” represents an integer of 0 to 10, and preferably represents 0 or 1. Constituting units in the formula (surf-1) does not stipulate their array order, and the units may be bonded in a block or randomly. Manufacturing method for the surfactant of partially fluorinated oxetane ring-opening polymerized product is described in detail in U.S. Pat. No. 5,650,483B.

In the ArF immersion lithography without a resist protective film, the surfactant insoluble or hardly soluble in water and soluble in an alkaline developer has a function of reducing penetration of water or leaching by orientation on the surface of the resist film. Thus, such a surfactant is useful for inhibiting elution of a water-soluble component from the resist film to reduce damage of an exposure apparatus. Such a surfactant is also useful because such a surfactant becomes soluble during development with an aqueous alkaline solution after the exposure or after post exposure bake (PEB), and hardly forms a foreign matter causing a defect. Such a surfactant, which has a property of being insoluble or hardly soluble in water and soluble in an alkaline developer, is preferably a polymer surfactant, which is also referred to as a hydrophobic resin. In particular, such a surfactant preferably has high water repellency and improves water-slipping property.

Specific examples of such a polymer surfactant include a polymer having at least one selected from repeating units represented by any one of the following formulae (6A) to (6E).

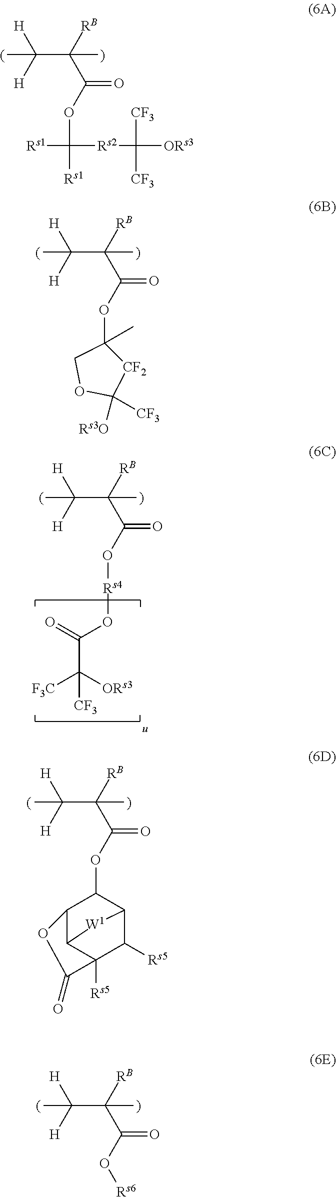

In the formulae (6A) to (6E), R^{B} represents a hydrogen atom, a fluorine atom, a methyl group, or a trifluoromethyl group. W¹ represents —CH₂—, —CH₂CH₂—, —O—, or separated two “—H”s. R^{s1} each independently represents a hydrogen atom or a hydrocarbyl group having 1 to 10 carbon atoms. R^{s2} represents a single bond or a linear or branched hydrocarbylene group having 1 to 5 carbon atoms. R^{s4} each independently represents a hydrogen atom, a hydrocarbyl group or fluorinated hydrocarbyl group having 1 to 15 carbon atoms, or an acid-labile group. When R^{s3} represents a hydrocarbyl group or a fluorinated hydrocarbyl group, an ether bond or a carbonyl group may be intervened between a carbon-carbon bond. R^{s4} represents a (u+1)-valent hydrocarbon group or fluorinated hydrocarbon group having 1 to 20 carbon atoms. “u” represents an integer of 1 to 3. R^{s5} each independently represents a hydrogen atom or a group represented by “—C(═O)—O—R^{sa}”. R^{sa} represents a fluorinated hydrocarbyl group having 1 to 20 carbon atoms. R^{s6} represents a hydrocarbyl group or fluorinated hydrocarbyl group having 1 to 15 carbon atoms, and an ether bond or a carbonyl group may intervene between a carbon-carbon bond thereof.

The hydrocarbyl group having 1 to 10 carbon atoms and represented by R^{s1} is preferably a saturated hydrocarbyl group, and may be linear, branched, or cyclic. Specific examples thereof include: alkyl groups having 1 to 10 carbon atoms, such as a methyl group, an ethyl group, an n-propyl group, an isopropyl group, an n-butyl group, an isobutyl group, a sec-butyl group, a tert-butyl group, a n-pentyl group, a n-hexyl group, a n-heptyl group, a n-octyl group, a n-nonyl group, and a n-decyl group; and cyclic saturated hydrocarbyl groups having 3 to 10 carbon atoms, such as a cyclopropyl group, a cyclobutyl group, a cyclopentyl group, a cyclohexyl group, an adamantyl group, and a norbornyl group. Among these, groups having 1 to 6 carbon atoms are preferable.

The hydrocarbylene group represented by R^{s2} is preferably saturated hydrocarbylene group, and may be linear, branched, or cyclic. Specific examples thereof include a methylene group, an ethylene group, a propylene group, a butylene group, and a pentylene group.

The hydrocarbyl group represented by R^{s3} or R^{s6} may be saturated or unsaturated, and may be linear, branched, or cyclic. Specific examples thereof include: saturated hydrocarbyl groups; and unsaturated aliphatic hydrocarbyl groups such as an alkenyl group and an alkynyl group. However, a saturated hydrocarbyl group is preferable. Specific examples of the saturated hydrocarbyl groups include, besides the groups exemplified as the hydrocarbyl group represented by Rel, an undecyl group, a dodecyl group, a tridecyl group, a tetradecyl group, and a pentadecyl group. Examples of the fluorinated hydrocarbyl group represented by R^{s3} or R^{s6} include groups in which a part or all of hydrogen atoms bonded to a carbon atom in the above hydrocarbyl group are substituted with a fluorine atom. As described above, an ether bond or a carbonyl group may intervene between a carbon-carbon bond thereof.

Specific examples of the acid-labile group represented by R^{s3} include: the groups represented by the aforementioned formulae (AL-1) to (AL-3); trialkylsilyl groups in which each alkyl group has 1 to 6 carbon atoms; and oxo group-containing alkyl groups having 4 to 20 carbon atoms.

The (u+1)-valent hydrocarbon group or fluorinated hydrocarbon group represented by R^{s4} may be linear, branched, or cyclic. Specific examples thereof include groups obtained by further removing “u” hydrogen atoms from the above hydrocarbyl group or the fluorinated hydrocarbyl group, or the like.

The fluorinated hydrocarbyl group represented by R^{sa} is preferably saturated, and may be linear, branched, or cyclic. Examples thereof include groups in which a part or all of hydrogen atoms of the above hydrocarbyl group are substituted with a fluorine atom. Specific examples thereof include a trifluoromethyl group, a 2,2,2-trifluoroethyl group, a 3,3,3-trifluoro-1-propyl group, a 3,3,3-trifluoro-2-propyl group, a 2,2,3,3-tetrafluoropropyl group, a 1,1,1,3,3,3-hexafluoroisopropyl group, a 2,2,3,3,4,4,4-heptafluorobutyl group, a 2,2,3,3,4,4,5,5-octafluoropentyl group, a 2,2,3,3,4,4,5,5,6,6,7,7-dodecafluoroheptyl group, a 2-(perfluorobutyl)ethyl group, a 2-(perfluorohexyl)ethyl group, a 2-(perfluorooctyl)ethyl group, and a 2-(perfluorodecyl)ethyl group.

Examples of the repeating units represented by any one of the formulae (6A) to (6E) include the following repeating units, but are not limited thereto. In the following formulae, R^{B} represents the same as defined above.

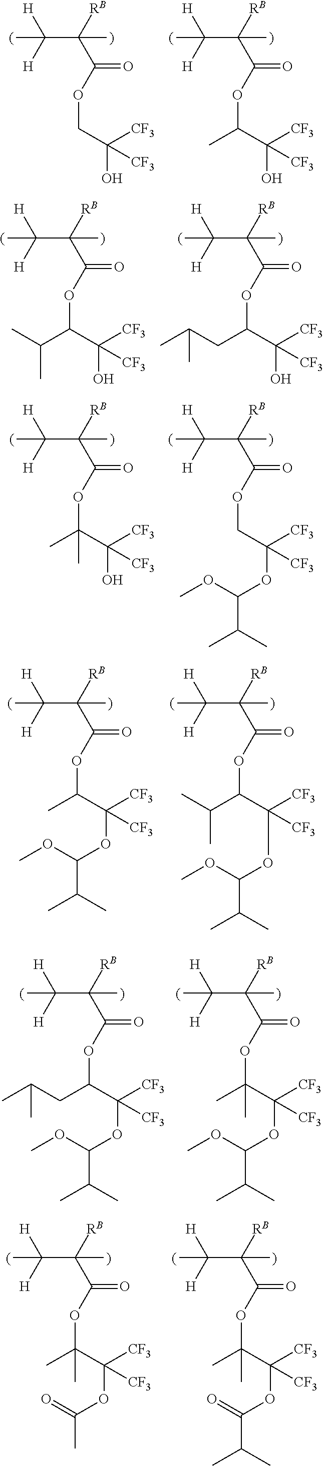

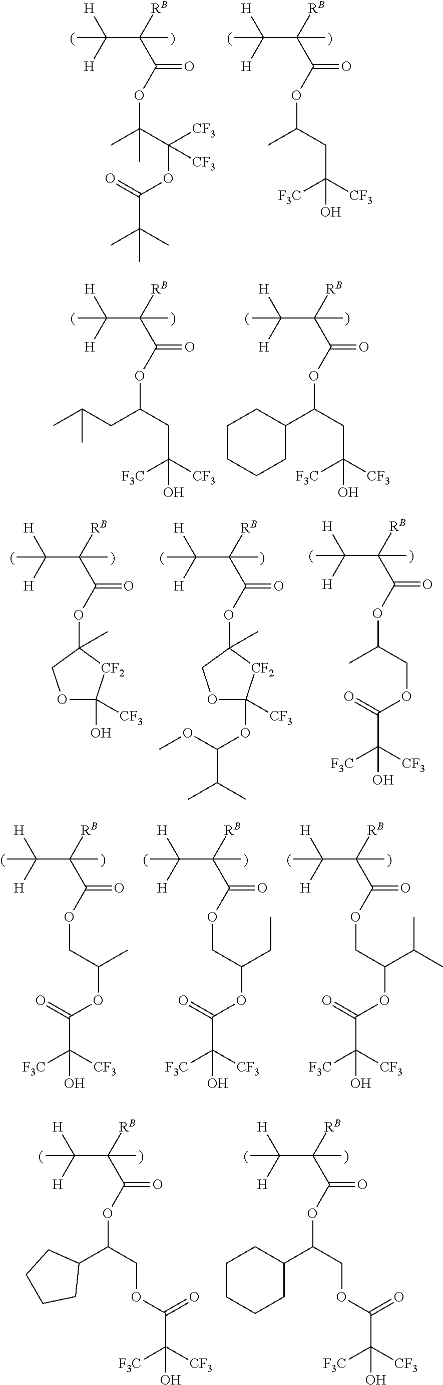

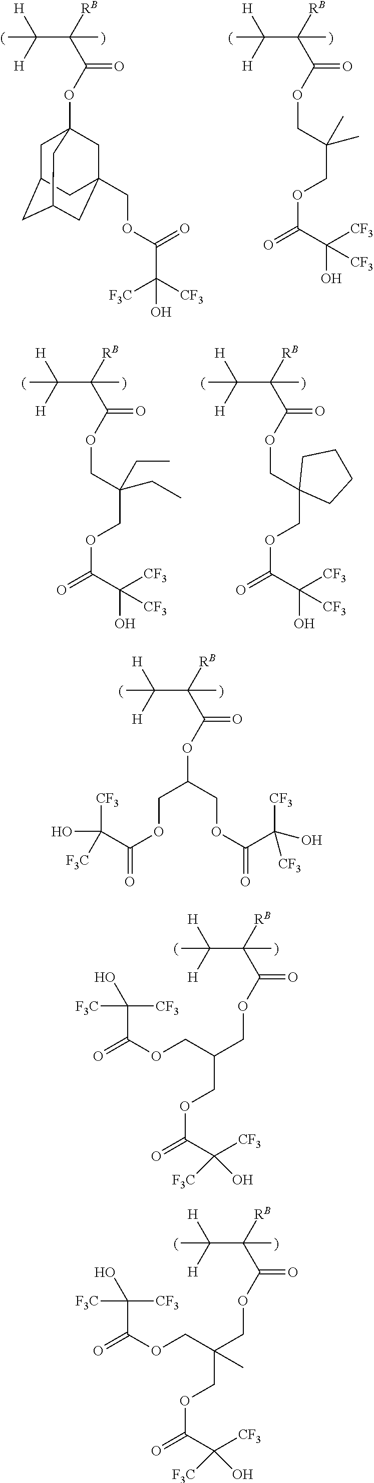

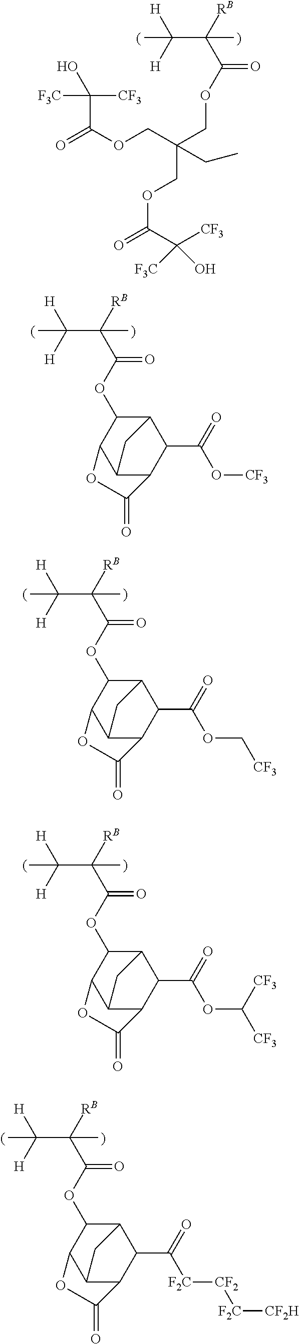

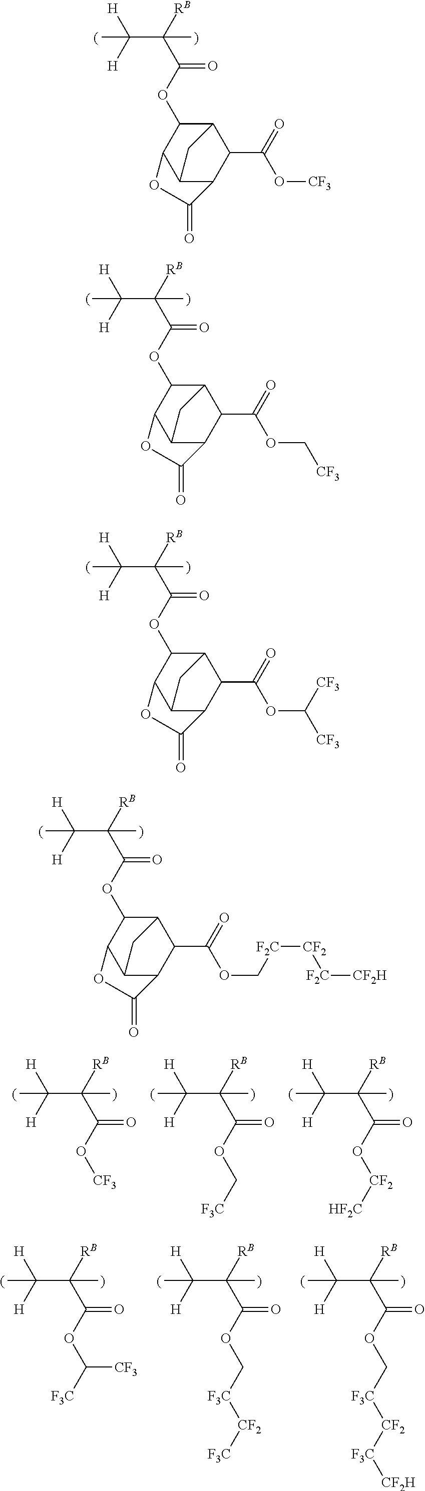

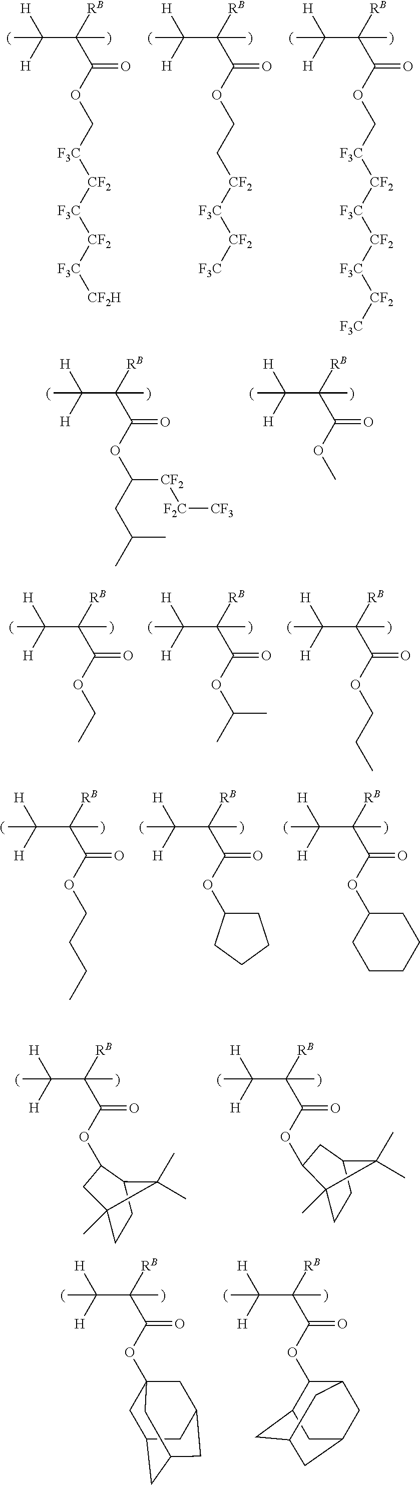

The polymer surfactant may further have a repeating unit other than the repeating units represented by the formulae (6A) to (6E). Specific examples of the other repeating unit include repeating units obtained from methacrylic acid, an α-trifluoromethylacrylic acid derivative, etc. In the polymer surfactant, the content of the repeating units represented by the formulae (6A) to (6E) is preferably 20 mol % or more, more preferably 60 mol % or more, and further preferably 100 mol % in all the repeating units.

Mw of the polymer surfactant is preferably 1,000 to 500,000, and more preferably 3,000 to 100,000. Mw/Mn is preferably 1.0 to 2.0, and more preferably 1.0 to 1.6.

Examples of a method for synthesizing the polymer surfactant include a method in which monomers having an unsaturated bond to yield the repeating unit represented by the formulae (6A) to (6E) and, as necessary, the other repeating unit were added with a radical initiator in an organic solvent and heated to be polymerized. Specific examples of the organic solvent used in the polymerization include toluene, benzene, THF, diethyl ether, and dioxane. Specific examples of the polymerization initiator include AIBN, 2,2′-azobis(2,4-dimethylvaleronitrile), dimethyl 2,2-azobis(2-methylpropionate), benzoyl peroxide, and lauroyl peroxide. The reaction temperature is preferably 50° C. to 100° C. The reaction time is preferably 4 to 24 hours. For the acid-labile group, those introduced into the monomer may be used as it is, or may be used after being protected or partially protected after the polymerization.

When the polymer surfactant is synthesized, known chain transfer agents, such as dodecyl mercaptan and 2-mercaptoethanol, may be used to regulate the molecular weight. In this case, the addition amount of these chain transfer agents is preferably 0.01 to 10 mol % relative to the total number of moles of the monomers to be polymerized.

When the inventive chemically-amplified resist composition contains the component (E) surfactant, the content thereof is preferably 0.1 to 50 parts by mass, and more preferably 0.5 to 10 parts by mass, relative to 80 parts by mass of the base polymer (component A) or (component A-1). When the content of the component (E) surfactant is 0.1 part by mass or more, the sweepback contact angle between the resist film surface and water is sufficiently improved. When the content is 50 parts by mass or less, the resist film surface has a low dissolution rate in the developer to sufficiently maintain the height of the formed fine pattern. One kind of the component (E) surfactants may be used, or two or more kinds thereof may be used in combination.

(Component B), (component C-1), and (component C-2) may each independently be added directly to the resist composition, or may be first dissolved in a good solvent to form a solution and then added thereto. Examples of good solvents include GBL, DAA, PGMEA, ethyl lactate, PGME, and mixed solvents of two or more kinds of these. In addition, after dissolving (component B), (component C-1), and (component C-2), the solution may be filtered and the filtered solution may be added thereto. Filtering can remove foreign matter and gels that may cause defects, which is effective in stabilizing quality.

Examples of the filter material used in the above-mentioned filter filtration include fluorocarbon-based, cellulose-based, nylon-based, polyester-based, and hydrocarbon-based materials. In the filtration process of the chemically-amplified resist composition, however, a filter made of a fluorocarbon-based material known as Teflon (registered trademark), a hydrocarbon-based material such as polyethylene or polypropylene, or a nylon-based material is preferable. The pore size of the filter can be appropriately selected according to the target cleanliness, but is preferably 100 nm or less, more preferably 20 nm or less, and further preferably 5 nm or less. In addition, one kind of these filters may be used, or two or more kinds thereof may be used in combination. As the filtration method, passing the solution through the filter may be only once, but it is more preferable to circulate the solution and perform filtration multiple times.

### [(F) Dissolution Inhibitor]

The inventive chemically-amplified resist composition may further contain a dissolution inhibitor as component (F). When the inventive chemically-amplified resist composition is a positive type, blending a dissolution inhibitor can further increase the difference in dissolution rate between the exposed portion and the unexposed portion, and further enhance the resolution.

Examples of the dissolution inhibitor include a compound which has a molecular weight of preferably 100 to 1,000, more preferably 150 to 800, and contains: two or more phenolic hydroxy groups per molecule in which 0 to 100 mol % of all the hydrogen atoms of such phenolic hydroxy groups are substituted with acid labile groups; or a carboxy group in a molecule in which 50 to 100 mol % of all the hydrogen atoms of such carboxyl groups are substituted with acid labile groups on average. Specific examples include compounds obtained by substituting acid labile groups for hydrogen atoms of hydroxy groups or carboxy groups of bisphenol A, trisphenol, phenolphthalein, cresol novolak, naphthalenecarboxylic acid, adamantanecarboxylic acid, and cholic acid. Examples of such compounds are disclosed in paragraphs [0155] to [0178] of JP2008-122932A.

When the inventive chemically-amplified resist composition contains the component (F) dissolution inhibitor, the dissolution inhibitor is contained in an amount of preferably 0 to 50 parts by mass, more preferably 5 to 40 parts by mass, based on 80 parts by mass of the base polymer (component A) or (component A-1). One kind of the (F) dissolution inhibitors may be used, or two or more kinds thereof may be used in combination.

### [(G) Other Components]

As another component (G), the inventive chemically-amplified resist composition may contain: a compound to be decomposed by an acid to generate an acid (acid amplifying compounds); an organic acid derivative; fluorine-substituted alcohol; a water-repellency enhancer; etc. As the acid amplifying compound, compounds described in JP2009-269953A or JP2010-215608A can be referred. When the acid amplifying compound is contained, the content thereof is preferably 0 to 5 parts by mass, and more preferably 0 to 3 parts by mass, relative to 80 parts by mass of the base polymer (component A) or (component A-1). If the content is too high, it is difficult to control the acid diffusion, and deterioration in resolution and deterioration in the pattern shape may occur. As the organic acid derivative and the fluorine-substituted alcohol, compounds described in JP2009-269953A or JP2010-215608A can be referred.

The water-repellency enhancer can be employed in immersion lithography with no top coat. The water-repellency enhancer is preferably a polymer containing a fluorinated alkyl group, a polymer containing a 1,1,1,3,3,3-hexafluoro-2-propanol residue with a particular structure, or the like, more preferably ones exemplified in JP2007-297590A, JP2008-111103A, or the like. The water-repellency enhancer needs to be dissolved in an alkaline developer or an organic solvent developer. The water-repellency enhancer having a particular 1,1,1,3,3,3-hexafluoro-2-propanol residue mentioned above has favorable solubility to developers. As a water-repellency enhancer, a polymer having a repeating unit having an amino group or an amine salt exhibits high effects of preventing acid evaporation during PEB and subsequent opening failure of a hole pattern after development. When the inventive chemically-amplified resist composition contains the water-repellency enhancer, the content thereof is preferably 0 to 20 parts by mass, more preferably 0.5 to 10 parts by mass, relative to 80 parts by mass of the base polymer (component A) or (component A-1).

### [Patterning Process]

When the inventive chemically-amplified resist composition is used for manufacturing various integrated circuits, known lithography techniques are applicable. For example, the patterning process includes steps of: applying and heating the above-described chemically-amplified resist composition on a substrate to form a resist film; exposing the resist film to a high-energy beam; and developing the exposed resist film by using a developer.

First, the inventive chemically-amplified resist composition is applied onto a substrate for manufacturing an integrated circuit (such as Si, SiO₂, SiN, SiON, TiN, WSi, BPSG, SOG, organic antireflective film) or a substrate for manufacturing a mask circuit (such as Cr, CrO, CrON, MoSi₂, SiO₂) by an appropriate coating process, such as spin coating, roll coating, flow coating, dip coating, spray coating, or doctor coating so that the coating film can have a thickness of 0.01 to 2.0 μm. The resultant is prebaked on a hot plate preferably at 60 to 150° C. for 10 seconds to 30 minutes, more preferably at 80 to 120° C. for 30 seconds to 20 minutes. In this manner, a resist film is formed.

Then, the resist film is exposed using a high-energy beam. Examples of the high-energy beam include ultraviolet ray, deep ultraviolet ray, EB, EUV having a wavelength of 3 to 15 nm, X-ray, soft X-ray, excimer laser beam, γ-ray, and synchrotron radiation. When ultraviolet ray, deep ultraviolet ray, EUV, X-ray, soft X-ray, excimer laser beam, γ-ray, synchrotron radiation, or the like is employed as the high-energy beam, the irradiation is performed directly or using a mask for forming a target pattern, at an exposure dose of preferably about 1 to 200 mJ/cm², more preferably about 10 to 100 mJ/cm². When EB is employed as the high-energy beam, the exposure dose is preferably about 0.1 to 100 μC/cm², more preferably about 0.5 to 50 μC/cm², and the writing is performed directly or using a mask for forming a target pattern. Among the high-energy beams, the inventive chemically-amplified resist composition is particularly suitable for fine patterning with an ArF excimer laser beam having a wavelength of 193 nm, an KrF excimer laser beam having a wavelength of 248 nm, an EB, an EUV having a wavelength of 3 to 15 nm, X-ray, soft X-ray, γ-ray, or synchrotron radiation.

The exposure may be followed by PEB on a hot plate preferably at 60 to 150° C. for 10 seconds to 30 minutes, more preferably at 80 to 120° C. for 30 seconds to 20 minutes.

After the exposure or PEB, development is performed using a developer of 0.1 to 10 mass %, preferably 2 to 5 mass % aqueous alkaline solution, such as tetramethylammonium hydroxide (TMAH), tetraethylammonium hydroxide, tetrapropylammonium hydroxide, and tetrabutylammonium hydroxide, for 3 seconds to 3 minutes, preferably 5 seconds to 2 minutes, by a conventional technique, such as a dip method, a puddle method, and a spray method. Thereby, the portion irradiated with the light is dissolved by the developer, while the unexposed portion remains undissolved. In this way, the target positive pattern is formed on the substrate.

Instead of the aqueous alkaline solution, organic solvent development can also be used for obtaining a negative pattern. Specific examples of the developer used in this event include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, phenylmethyl acetate, benzyl formate, phenylethyl formate, methyl 3-phenylpropionate, benzyl propionate, phenylethyl acetate, and 2-phenylethyl acetate. One kind of these organic solvents may be used, or two or more kinds thereof may be used in mixture.

When the development is completed, rinsing may be performed. The rinsing liquid is preferably a solvent that is miscible with the developer but does not dissolve the resist film. As such a solvent, it is preferable to use an alcohol having 3 to 10 carbon atoms, an ether compound having 8 to 12 carbon atoms, and an alkane, alkene, alkyne and aromatic solvent, each having 6 to 12 carbon atoms.

Specific examples of the alcohol having 3 to 10 carbon atoms include n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, and 1-octanol.

Examples of the ether compound having 8 to 12 carbon atoms include di-n-butyl ether, diisobutyl ether, di-sec-butyl ether, di-n-pentyl ether, diisopentyl ether, di-sec-pentyl ether, di-tert-pentyl ether, and di-n-hexyl ether.

Examples of the alkane having 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, and cyclononane. Examples of the alkene having 6 to 12 carbon atoms include hexene, heptene, octene, cyclohexene, methylcyclohexene, dimethylcyclohexene, cycloheptene, and cyclooctene. Examples of the alkyne having 6 to 12 carbon atoms include hexyne, heptyne, and octyne.

Examples of the aromatic solvent include toluene, xylene, ethylbenzene, isopropylbenzene, tert-butylbenzene, and mesitylene.

The rinsing can reduce occurrence of resist pattern collapse and defect formation. Meanwhile, the rinsing is not necessarily essential, and the amount of the solvent used can be reduced by not performing the rinsing.

After the development, a hole pattern or a trench pattern can be shrunk by thermal flow, RELACS process, or DSA process. A shrink agent is applied onto the hole pattern, and the shrink agent undergoes crosslinking on the surface of the resist film by diffusion of the acid catalyst from the resist film during baking, so that the shrink agent is attached to sidewalls of the hole pattern. The baking temperature is preferably 70 to 180° C., more preferably 80 to 170° C. The baking time is preferably 10 to 300 seconds. The extra shrink agent is removed, and the hole pattern is shrunk.

As a particularly preferable patterning process, the present invention provides a patterning process including: applying the resist composition described above onto a substrate; heat-treating the resist composition applied on the substrate; exposing the resist composition to light of a KrF excimer laser beam, an ArF excimer laser beam, an electron beam, or EUV light; and developing the resist composition with a developer.

In this case, by using an aqueous alkaline solution as a developer, it is possible to obtain a positive pattern in which the exposed portion is dissolved and the unexposed portion is not dissolved. Alternatively, by using an organic solvent as a developer, it is possible to obtain a negative pattern in which the unexposed portion is dissolved and the exposed portion is not dissolved.

### EXAMPLE

Hereinafter, the present invention will be specifically described with reference to Examples and Comparative Examples. However, the present invention is not limited thereto.

### [1] Synthesis of Quencher

### [Example 1-1] Synthesis of Quencher (Q-1)

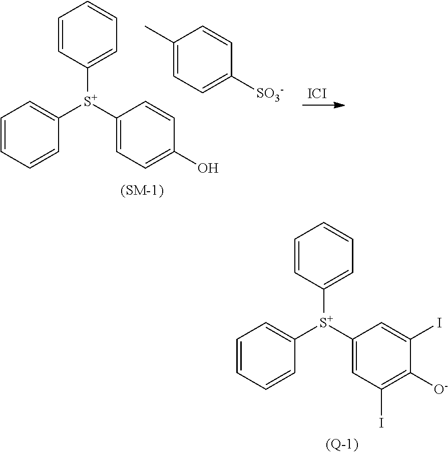

A mixed solution of 28.8 g of iodine monochloride and 58 g of methylene chloride was added dropwise to a mixed solution of 72 g of sulfonium salt (SM-1), 36.8 g of potassium carbonate, 320 g of methylene chloride, and 120 g of methanol under ice cooling, and the mixture was stirred at room temperature overnight. Thereafter, the reaction solution was ice-cooled, added with 455 g of 2.5% hydrochloric acid dropwise, and stirred, thus to fractionate an organic layer. The obtained organic layer was subjected to liquid-liquid separation and washing with 200 g of pure water, 216 g of an aqueous sodium sulfite solution, and 200 g of pure water, in this sequential order. 60 g of methyl isobutyl ketone was added to a solid obtained by concentrating the organic layer under reduced pressure and stirred. The solid was filtered out and dried under reduced pressure to obtain 41.5 g of the target quencher (Q-1) as a pale yellow solid (yield 88%).

The spectrum data on the obtained sulfonium salt are shown below.

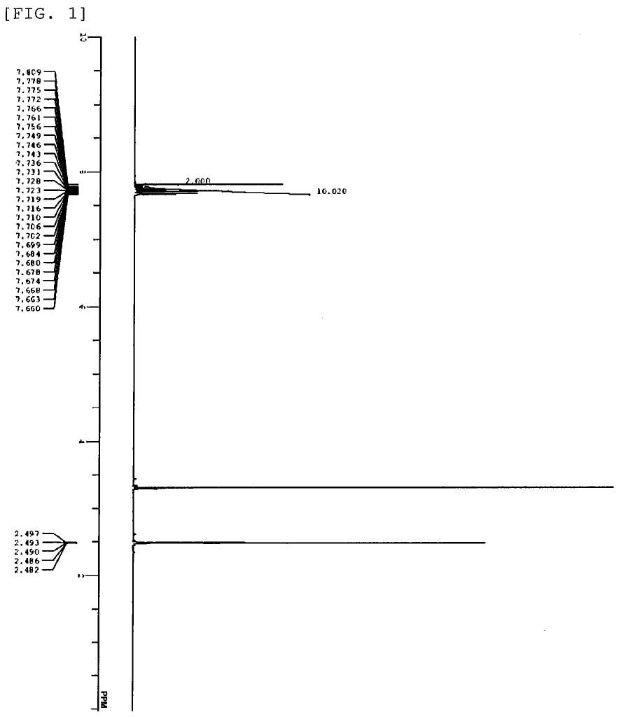

Nuclear magnetic resonance spectra (H-NMR/500 MHz/DMSO-d₆): FIG. 1

Infrared absorption spectrum (D-ATR; cm⁻¹)

3082, 3057, 3046, 3031, 2419, 1779, 1745, 1580, 1539, 1525, 1491, 1472, 1441, 1335, 1312, 1278, 1181, 1144, 1101, 1078, 1021, 993, 927, 893, 875, 834, 750, 685, 611, 579, 549, 513, 478, 461, 430 cm⁻¹

### [Examples 1-2 to 1-12] Synthesis of Quenchers (Q-2) to (Q-12)

Quenchers (Q-2) to (Q-12) represented by the following formulae were synthesized in the same manner as in (Q-1) above, using the corresponding materials and known organic synthesis reactions.

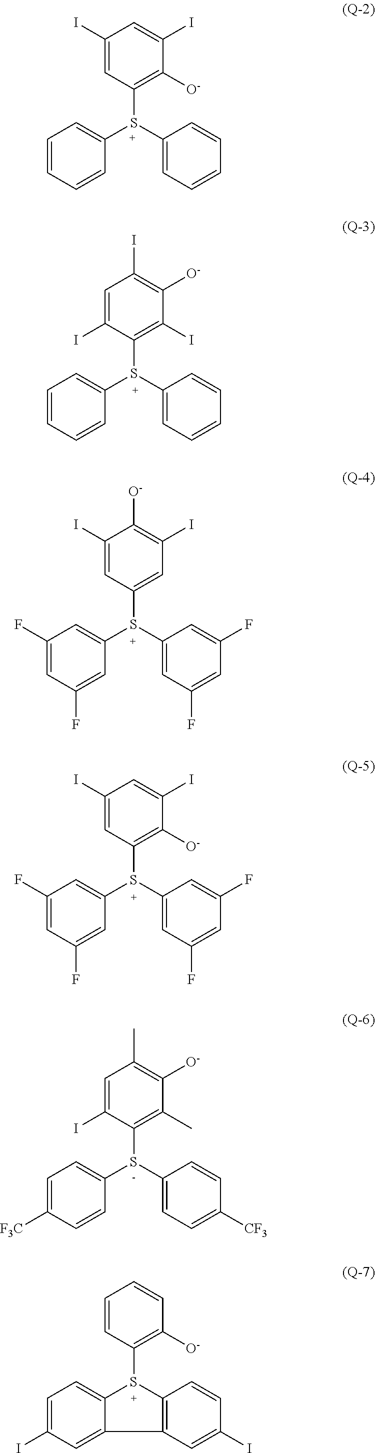

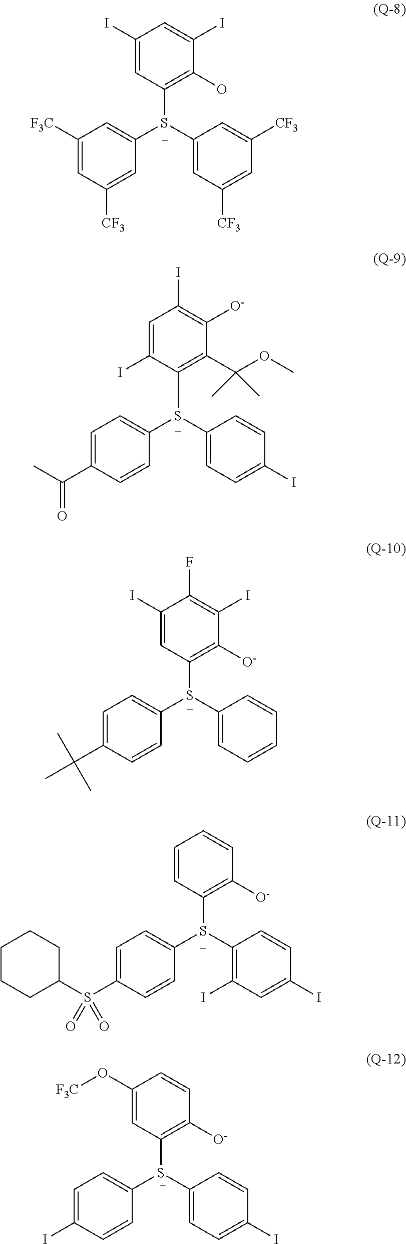

### [Comparative Examples 1-1 to 1-6] Synthesis of Comparative Quenchers (Qc-1) to (Qc-6)

Quenchers (Qc-1) to (Qc-6) represented by the following formulae were synthesized in the same manner as in (Q-1) above, using the corresponding materials and known organic synthesis reactions.

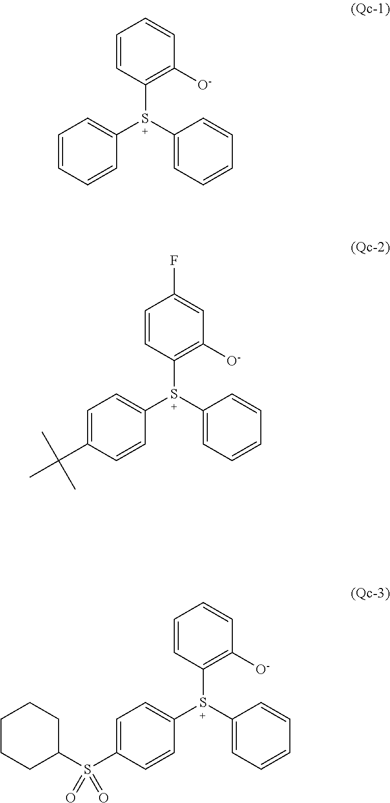

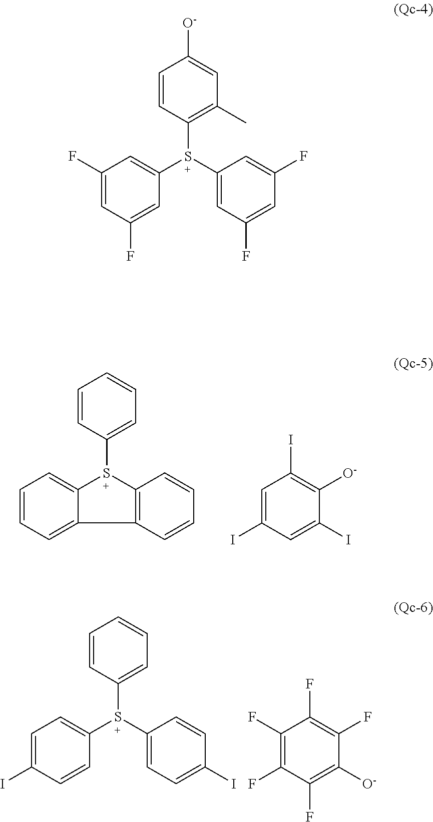

### [2] Synthesis of Polymer Compounds

### [Example 2-1] Synthesis of Polymer Compound (P-1)

Under a nitrogen atmosphere, a monomer-polymerization initiator solution was prepared using 22 g of 1-tert-butylcyclopentyl methacrylic acid, 17 g of 2-oxotetrahydrofuran-3-yl methacrylic acid, 0.49 g of V-601 (manufactured by Wako Pure Chemical Industries, Ltd.), 0.40 g of 2-mercaptoethanol, and 50 g of methyl ethyl ketone. 23 g of methyl ethyl ketone was added into another flask with a nitrogen atmosphere, heated to 80° C. with stirring, and then the monomer-polymerization initiator solution above was added dropwise over 4 hours. After the dropwise addition, the polymerization liquid was further stirred for 2 hours with maintaining the temperature at 80° C. and then cooled to a room temperature. The obtained polymerization liquid was added dropwise to 640 g of vigorously stirred methanol, and a precipitated copolymer was filtered out. The copolymer was washed twice with 240 g of methanol and dried in a vacuum at 50° C. for 20 hours to obtain 36 g of white powder copolymer (P-1) (yield: 90%). The copolymer (P-1) had a weight-average molecular weight (Mw) of 7,800 in terms of polystyrene and a dispersity (Mw/Mn) of 1.80, when analyzed by GPC.

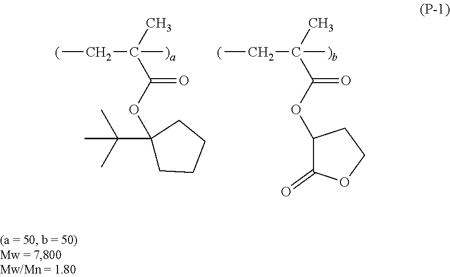

### [Examples 2-2 to 2-10] Synthesis of Polymer Compounds (P-2 to p-10)

The following polymer compounds were prepared in the same manner as in the above [Example 2-1], except that the types and mixing ratios of monomers each were changed.

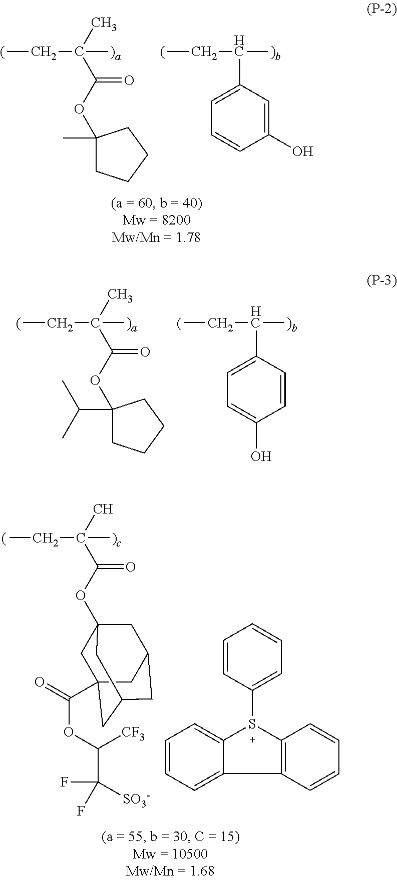

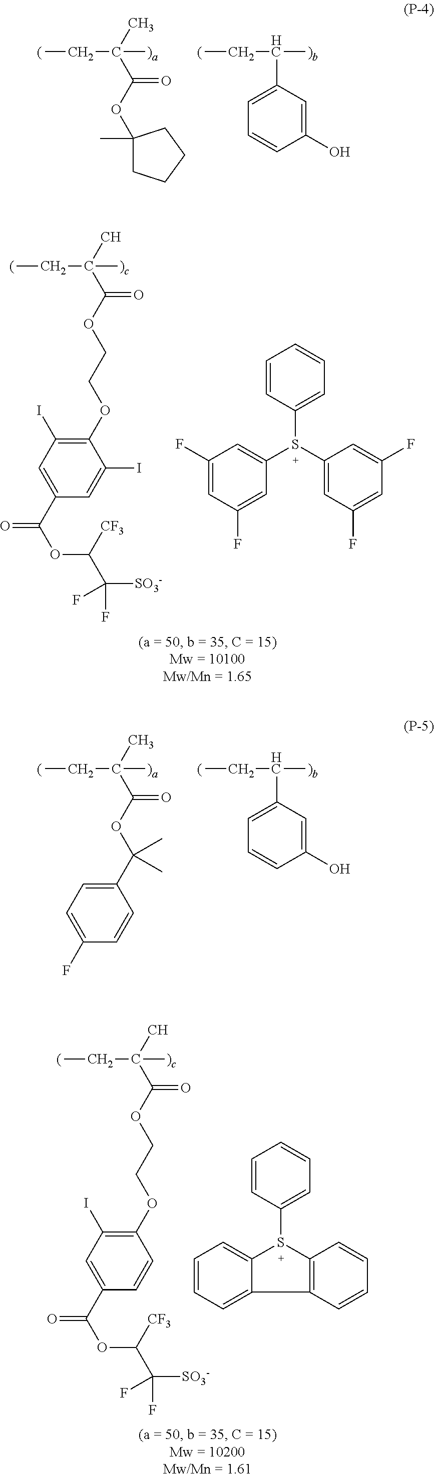

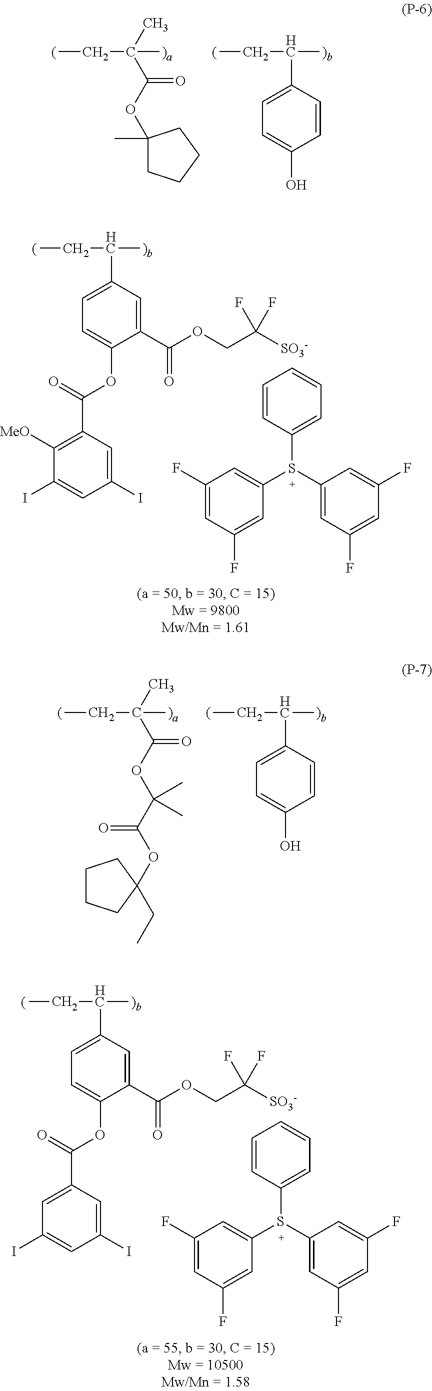

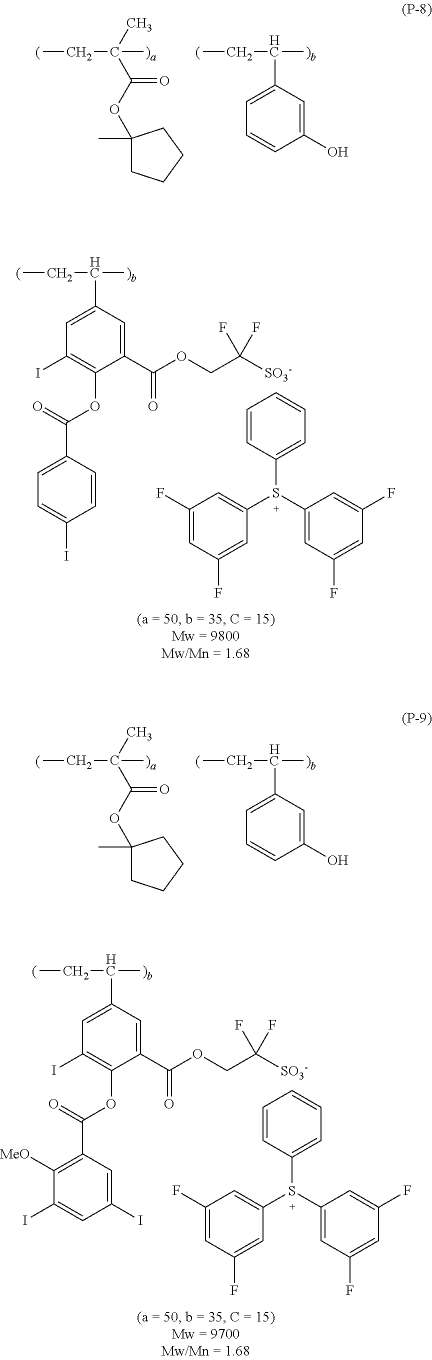

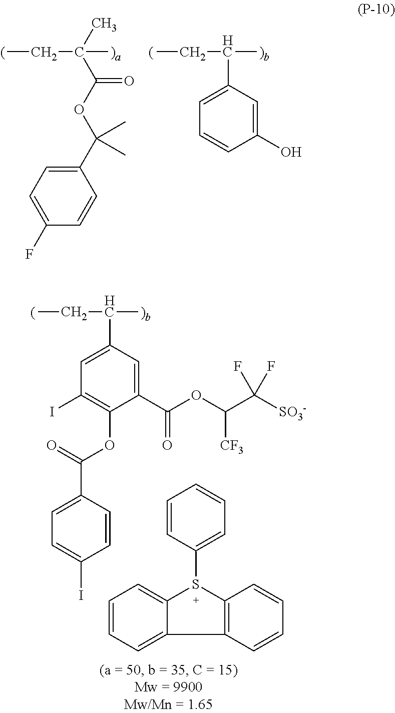

### [3] Preparation of Chemically-Amplified Resist Compositions

### Examples 3-1 to 3-48, Comparative Examples 3-1 to 3-24

The acid diffusion inhibitors (Q-1 to Q-12) and the polymer compounds (P-1 to P-10) described in the above Examples, and further, if necessary, the photo-acid generators (PAG-1 to PAG-2), the acid diffusion inhibitor (QA-1) other than the salts having the structure represented by the general formula (1) above were dissolved in a solvent containing 0.01 mass % of surfactant Polyfox 636 (manufactured by Omnova) to make a resist material. The resist material was further filtered through a 0.2 μm Teflon (registered trademark) filter to prepare each resist solution. In addition, for comparison, resist solutions were prepared in the same manner as above using the acid diffusion inhibitor (Qc-1 to Qc-6). The compositions of the resist solutions prepared are shown in Tables 1 to 7 below.

In Tables 1 to 7, the photo-acid generators (PAG-1 to PAG-2), acid diffusion inhibitor (QA-1) other than the salt having the structure represented by the formula (1), and solvents used in the resist compositions are as follows.

### Photo-Acid Generator (PAG-1 to PAG-2)

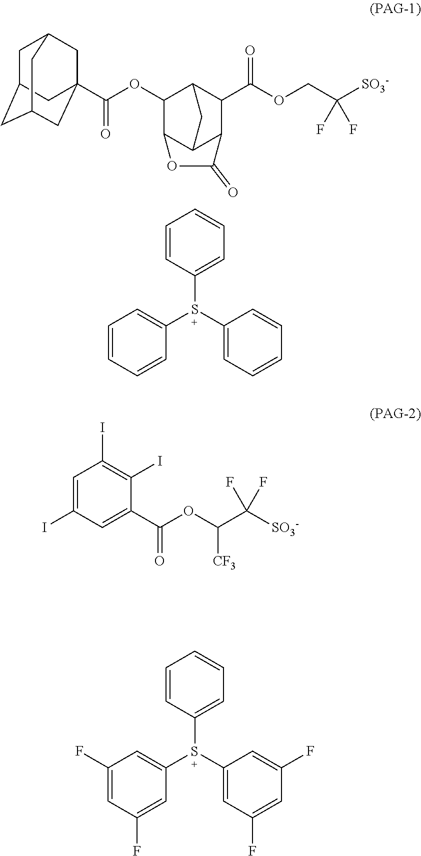

### Acid Diffusion Inhibitor (QA-1)

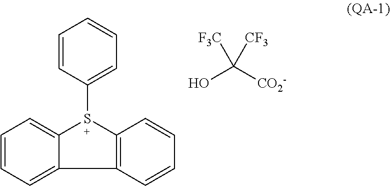

### Solvent

PGMEA: Propylene glycol monomethyl ether acetate
GBL: γ-butyrolactone
DAA: Diacetone alcohol

TABLE 1

| | | Resin | Acid generator | Quencher | Solvent |
|---|---|---|---|---|---|
| | Resist | (parts by mass) | (parts by mass) | (parts by mass) | (parts by mass) |
| Example | R-1 | P-1 | PAG-1 | Q-1 | PGMEA / GBL |
| 3-1 | | (100) | (8.0) | (6.0) | (1,920 / 480) |
| Example | R-2 | P-1 | PAG-1 | Q-2 | PGMEA / GBL |
| 3-2 | | (100) | (8.0) | (6.0) | (1,920 / 480) |
| Example | R-3 | P-1 | PAG-1 | Q-5 | PGMEA / GBL |
| 3-3 | | (100) | (8.0) | (6.0) | (1,920 / 480) |
| Example | R-4 | P-1 | PAG-1 | Q-7 | PGMEA / GBL |
| 3-4 | | (100) | (8.0) | (5.5) | (1,920 / 480) |
| Example | R-5 | P-1 | PAG-1 | Q-8 | PGMEA / GBL |
| 3-5 | | (100) | (8.0) | (5.2) | (1,920 / 480) |
| Example | R-6 | P-1 | PAG-1 | Q-12 | PGMEA / GBL |
| 3-6 | | (100) | (8.0) | (5.5) | (1,920 / 480) |

TABLE 2

| | | Resin | Acid generator | Quencher | Solvent |
|---|---|---|---|---|---|
| | Resist | (parts by mass) | (parts by mass) | (parts by mass) | (parts by mass) |
| Example | R-7 | P-2 | PAG-2 | Q-1 | PGMEA / DAA |
| 3-7 | | (100) | (20.0) | (4.5) | (2,100 / 900) |
| Example | R-8 | P-2 | PAG-2 | Q-2 | PGMEA / DAA |
| 3-8 | | (100) | (20.0) | (4.5) | (2,100 / 900) |
| Example | R-9 | P-2 | PAG-2 | Q-3 | PGMEA / DAA |
| 3-9 | | (100) | (20.0) | (4.5) | (2,100 / 900) |
| Example | R-10 | P-2 | PAG-2 | Q-4 | PGMEA / DAA |
| 3-10 | | (100) | (20.0) | (4.5) | (2,100 / 900) |
| Example | R-11 | P-2 | PAG-2 | Q-5 | PGMEA / DAA |
| 3-11 | | (100) | (20.0) | (4.5) | (2,100 / 900) |
| Example | R-12 | P-2 | PAG-2 | Q-7 | PGMEA / DAA |
| 3-12 | | (100) | (20.0) | (4.5) | (2,100 / 900) |
| Example | R-13 | P-2 | PAG-2 | Q-8 | PGMEA / DAA |
| 3-13 | | (100) | (20.0) | (4.5) | (2,100 / 900) |
| Example | R-14 | P-2 | PAG-2 | Q-10 | PGMEA / DAA |
| 3-14 | | (100) | (20.0) | (4.5) | (2,100 / 900) |
| Example | R-15 | P-2 | PAG-2 | Q-11 | PGMEA / DAA |
| 3-15 | | (100) | (20.0) | (4.5) | (2,100 / 900) |
| Example | R-16 | P-2 | PAG-2 | Q-12 | PGMEA / DAA |
| 3-16 | | (100) | (20.0) | (4.5) | (2,100 / 900) |

TABLE 3

| | | Resin | Acid generator | Quencher | Solvent |
|---|---|---|---|---|---|
| | Resist | (parts by mass) | (parts by mass) | (parts by mass) | (parts by mass) |
| Example | R-17 | P-3 | — | Q-1 | PGMEA / DAA |
| 3-17 | | (100) | | (4.5) | (2,100 / 900) |
| Example | R-18 | P-3 | — | Q-2 | PGMEA / DAA |
| 3-18 | | (100) | | (4.5) | (2,100 / 900) |
| Example | R-19 | P-3 | — | Q-3 | PGMEA / DAA |
| 3-19 | | (100) | | (4.5) | (2,100 / 900) |
| Example | R-20 | P-3 | — | Q-4 | PGMEA / DAA |
| 3-20 | | (100) | | (4.5) | (2,100 / 900) |
| Example | R-21 | P-3 | — | Q-5 | PGMEA / DAA |
| 3-21 | | (100) | | (4.5) | (2,100 / 900) |
| Example | R-22 | P-3 | — | Q-6 | PGMEA / DAA |
| 3-22 | | (100) | | (4.5) | (2,100 / 900) |
| Example | R-23 | P-3 | — | Q-7 | PGMEA / DAA |
| 3-23 | | (100) | | (4.5) | (2,100 / 900) |
| Example | R-24 | P-3 | — | Q-8 | PGMEA / DAA |
| 3-24 | | (100) | | (4.5) | (2,100 / 900) |
| Example | R-25 | P-3 | — | Q-9 | PGMEA / DAA |
| 3-25 | | (100) | | (4.5) | (2,100 / 900) |
| Example | R-26 | P-3 | — | Q-10 | PGMEA / DAA |
| 3-26 | | (100) | | (4.5) | (2,100 / 900) |
| Example | R-27 | P-3 | — | Q-11 | PGMEA / DAA |
| 3-27 | | (100) | | (4.5) | (2,100 / 900) |
| Example | R-28 | P-3 | — | Q-12 | PGMEA / DAA |
| 3-28 | | (100) | | (4.5) | (2,100 / 900) |
| Example | R-29 | P-4 | — | Q-3 | PGMEA / DAA |
| 3-29 | | (100) | | (4.5) | (2,100 / 900) |
| Example | R-30 | P-4 | — | Q-4 | PGMEA / DAA |
| 3-30 | | (100) | | (4.5) | (2,100 / 900) |
| Example | R-31 | P-4 | — | Q-5 | PGMEA / DAA |
| 3-31 | | (100) | | (4.5) | (2,100 / 900) |
| Example | R-32 | P-4 | — | Q-8 | PGMEA / DAA |
| 3-32 | | (100) | | (4.5) | (2,100 / 900) |
| Example | R-33 | P-4 | — | Q-12 | PGMEA / DAA |
| 3-33 | | (100) | | (4.5) | (2,100 / 900) |
| Example | R-34 | P-5 | — | Q-3 | PGMEA / DAA |
| 3-34 | | (100) | | (4.5) | (2,100 / 900) |

TABLE 4

| | | Resin | Acid generator | Quencher | Solvent |
|---|---|---|---|---|---|
| | Resist | (parts by mass) | (parts by mass) | (parts by mass) | (parts by mass) |
| Example | R-35 | P-5 | — | Q-4 | PGMEA / DAA |
| 3-35 | | (100) | | (4.5) | (2,100 / 900) |
| Example | R-36 | P-5 | — | Q-5 | PGMEA / DAA |
| 3-36 | | (100) | | (4.5) | (2,100 / 900) |
| Example | R-37 | P-5 | — | Q-8 | PGMEA / DAA |
| 3-37 | | (100) | | (4.5) | (2,100 / 900) |
| Example | R-38 | P-6 | — | Q-3 | PGMEA / DAA |
| 3-38 | | (100) | | (4.5) | (2,100 / 900) |
| Example | R-39 | P-6 | — | Q-4 | PGMEA / DAA |
| 3-39 | | (100) | | (4.5) | (2,100 / 900) |
| Example | R-40 | P-7 | — | Q-5 | PGMEA / DAA |
| 3-40 | | (100) | | (4.5) | (2,100 / 900) |
| Example | R-41 | P-7 | — | Q-8 | PGMEA / DAA |
| 3-41 | | (100) | | (4.5) | (2,100 / 900) |
| Example | R-42 | P-8 | — | Q-5 | PGMEA / DAA |
| 3-42 | | (100) | | (4.5) | (2,100 / 900) |
| Example | R-43 | P-8 | — | Q-8 | PGMEA / DAA |
| 3-43 | | (100) | | (4.5) | (2,100 / 900) |
| Example | R-44 | P-9 | — | Q-5 | PGMEA / DAA |
| 3-44 | | (100) | | (4.5) | (2,100 / 900) |
| Example | R-45 | P-9 | — | Q-8 | PGMEA / DAA |
| 3-45 | | (100) | | (4.5) | (2,100 / 900) |
| Example | R-46 | P-10 | — | Q-5 | PGMEA / DAA |
| 3-46 | | (100) | | (4.5) | (2,100 / 900) |
| Example | R-47 | P-10 | — | Q-8 | PGMEA / DAA |
| 3-47 | | (100) | | (4.5) | (2,100 / 900) |
| Example | R-48 | P-8 | PAG-2 | Q-5 (2.5) | PGMEA / DAA |
| 3-48 | | (100) | (3.0) | QA-1 (1.9) | (2,100 / 900) |

TABLE 5

| | | Resin | Acid generator | Quencher | Solvent |
|---|---|---|---|---|---|
| | Resist | (parts by mass) | (parts by mass) | (parts by mass) | (parts by mass) |
| Comparative | RC-1 | P-1 | PAG-1 | Qc-1 | PGMEA / GBL |
| Example | | (100) | (8.0) | (6.0) | (1,920 / 480) |
| 3-1 | | | | | |
| Comparative | RC-2 | P-1 | PAG-1 | Qc-2 | PGMEA / GBL |
| Example | | (100) | (8.0) | (6.0) | (1,920 / 480) |
| 3-2 | | | | | |
| Comparative | RC-3 | P-1 | PAG-1 | Qc-3 | PGMEA / GBL |
| Example | | (100) | (8.0) | (6.0) | (1,920 / 480) |
| 3-3 | | | | | |
| Comparative | RC- 4 | P-1 | PAG-1 | Qc-4 | PGMEA / GBL |
| Example | | (100) | (8.0) | (6.0) | (1,920 / 480) |
| 3-4 | | | | | |
| Comparative | RC-5 | P-1 | PAG-1 | Qc-5 | PGMEA / GBL |
| Example | | (100) | (8.0) | (6.0) | (1,920 / 480) |
| 3-5 | | | | | |
| Comparative | RC-6 | P-1 | PAG-1 | Qc-6 | PGMEA / GBL |
| Example | | (100) | (8.0) | (5.9) | (1,920 / 480) |
| 3-6 | | | | | |

TABLE 6

| | | Resin | Acid generator | Quencher | Solvent |
|---|---|---|---|---|---|
| | Resist | (parts by mass) | (parts by mass) | (parts by mass) | (parts by mass) |
| Comparative | RC-7 | P-2 | PAG-2 | Qc-1 | PGMEA / DAA |
| Example | | (100) | (20.0) | (4.5) | (2,100 / 900) |
| 3-7 | | | | | |
| Comparative | RC-8 | P-2 | PAG-2 | Qc-2 | PGMEA / DAA |
| Example | | (100) | (20.0) | (4.5) | (2,100 / 900) |
| 3-8 | | | | | |
| Comparative | RC-9 | P-2 | PAG-2 | Qc-3 | PGMEA / DAA |
| Example | | (100) | (20.0) | (4.5) | (2,100 / 900) |
| 3-9 | | | | | |
| Comparative | RC-10 | P-2 | PAG-2 | Qc-4 | PGMEA / DAA |
| Example | | (100) | (20.0) | (4.5) | (2,100 / 900) |
| 3-10 | | | | | |
| Comparative | RC-11 | P-2 | PAG-2 | Qc-5 | PGMEA / DAA |
| Example | | (100) | (20.0) | (4.5) | (2,100 / 900) |
| 3-11 | | | | | |
| Comparative | RC-12 | P-2 | PAG-2 | Qc-6 | PGMEA / DAA |
| Example | | (100) | (20.0) | (4.5) | (2,100 / 900) |
| 3-12 | | | | | |

TABLE 7

| | | Resin | Acid generator | Quencher | Solvent |
|---|---|---|---|---|---|
| | Resist | (parts by mass) | (parts by mass) | (parts by mass) | (parts by mass) |
| Comparative | RC-13 | P-3 | — | Qc-1 | PGMEA / DAA |
| Example | | (100) | | (4.5) | (2,100 / 900) |
| 3-13 | | | | | |
| Comparative | RC-14 | P-3 | — | Qc-2 | PGMEA / DAA |
| Example | | (100) | | (4.5) | (2,100 / 900) |
| 3-14 | | | | | |
| Comparative | RC-15 | P-3 | — | Qc-3 | PGMEA / DAA |
| Example | | (100) | | (4.5) | (2,100 / 900) |
| 3-15 | | | | | |
| Comparative | RC-16 | P-3 | — | Qc-4 | PGMEA / DAA |
| Example | | (100) | | (4.5) | (2,100 / 900) |
| 3-16 | | | | | |
| Comparative | RC-17 | P-3 | — | Qc-5 | PGMEA / DAA |
| Example | | (100) | | (4.5) | (2,100 / 900) |
| 3-17 | | | | | |
| Comparative | RC-18 | P-3 | — | Qc-6 | PGMEA / DAA |
| Example | | (100) | | (4.5) | (2,100 / 900) |
| 3-18 | | | | | |
| Comparative | RC-19 | P-4 | — | Qc-1 | PGMEA / DAA |
| Example | | (100) | | (4.5) | (2,100 / 900) |
| 3-19 | | | | | |
| Comparative | RC-20 | P-4 | — | Qc-2 | PGMEA / DAA |
| Example | | (100) | | (4.5) | (2,100 / 900) |
| 3-20 | | | | | |
| Comparative | RC-21 | P-4 | — | Qc-3 | PGMEA / DAA |
| Example | | (100) | | (4.5) | (2,100 / 900) |
| 3-21 | | | | | |
| Comparative | RC-22 | P-8 | — | Qc-4 | PGMEA / DAA |
| Example | | (100) | | (4.5) | (2,100 / 900) |
| 3-22 | | | | | |
| Comparative | RC-23 | P-8 | — | Qc-5 | PGMEA / DAA |
| Example | | (100) | | (4.5) | (2,100 / 900) |
| 3-23 | | | | | |
| Comparative | RC-24 | P-8 | — | Qc-6 | PGMEA / DAA |
| Example | | (100) | | (4.5) | (2,100 / 900) |
| 3-24 | | | | | |

### Examples 4-1 to 4-6 and Comparative Examples 4-1 to 4-6

### ArF Exposure Patterning Evaluation

A silicon substrate was coated with an antireflective film solution (ARC-29A manufactured by Nissan Chemical Co., Ltd.) and baked at 180° C. for 60 seconds to form an antireflective film (film thickness: 100 nm). The antireflective film was spin-coated with one of the resist compositions (R-1 to R-6 and RC-1 to RC-6), baked using a hot plate at 100° C. for 60 seconds to form a resist film having a film thickness of 90 nm. This was subjected to immersion exposure with a LS pattern having an on-wafer line width of 40 nm and pitch of 80 nm using an ArF immersion excimer laser scanner (NSR-S610C manufactured by Nikon Corporation, NA: 1.30, σ: 0.94/0.74, 350 dipole illumination, 6% halftone phase shift mask) while changing the exposure dose and focus. Incidentally, water was used as the immersion liquid. Subsequently, the film was baked (PEB) at 90° C. for 60 seconds, and development was performed with a 2.38 mass % TMAH aqueous solution for 60 seconds, to form a line-and-space (LS) pattern.

The LS patterns after the development were observed with CD-SEM (CG6300) manufactured by Hitachi High-Technologies Corporation. Sensitivity and line width roughness (LWR) were evaluated according to the following methods. Table 8 shows the results.

### [Sensitivity Evaluation]

As the sensitivity, an optimum exposure dose Eₒₚ (mJ/cm²) was determined at which an LS pattern with a line width of 40 nm and a pitch of 80 nm was obtained. The smaller this value, the higher the sensitivity.

### [LWR Evaluation]

The size of the LS pattern obtained by the irradiation at Eₒₚ was measured at ten positions in the longitudinal direction of the line. Based on this result, the triple value (3σ) of the standard deviation (σ) was determined as LWR. The smaller this value, the smaller the roughness and the more uniform the line width of the obtained pattern.

In this evaluation, the rating was judged as “Good” when the value is 2.5 nm or less, and judged as “Bad” when the value is more than 2.5 nm.

TABLE 8

| | Resist | Eop | |
|---|---|---|---|
| | composition | (mJ / cm²) | LWR (nm) |
| Example 4-1 | R-1 | 52 | Good (2.4) |
| Example 4-2 | R-2 | 52 | Good (2.4) |
| Example 4-3 | R-3 | 53 | Good (2.3) |
| Example 4-4 | R-4 | 50 | Good (2.2) |
| Example 4-5 | R-5 | 52 | Good (2.4) |
| Example 4-6 | R-6 | 53 | Good (2.3) |
| Comparative | RC-1 | 48 | Bad (3.1) |
| Example 4-1 | | | |
| Comparative | RC-2 | 51 | Bad (2.9) |
| Example 4-2 | | | |
| Comparative | RC-3 | 52 | Bad (2.9) |
| Example 4-3 | | | |
| Comparative | RC-4 | 52 | Bad (3.1) |
| Example 4-4 | | | |
| Comparative | RC-5 | 54 | Bad (3.4) |
| Example 4-5 | | | |
| Comparative | RC-6 | 54 | Bad (3.2) |
| Example 4-6 | | | |

The results in Table 8 showed that Examples 4-1 to 4-6, which used resist compositions containing the inventive sulfonium salt, had good sensitivity and excellent LWR, and were suitable as material for ArF immersion lithography. On the other hand, Comparative Examples 4-1 to 4-6, which used resist compositions containing a sulfonium salt different from that of the present invention, showed poor results in LWR.

### Examples 5-1 to 5-42 and Comparative Examples 5-1 to 5-18

### EUV Exposure Evaluation

Each of the resist materials (R-7 to R-48 and RC-7 to RC-24) was applied by spin coating onto a silicon substrate on which silicon-containing spin-on hard mask SHB-A940 (43 mass % of the silicon content) manufactured by Shin-Etsu Chemical Co. was formed with a film thickness of 20 nm, and then prebaked using a hot plate at 105° C. for 60 seconds to prepare a resist film with a film thickness of 50 nm. This was subjected to exposure with a LS pattern having an on-wafer size of 24 nm and a pitch of 48 nm using an EUV scanner NXE3400 (NA=0.33, σ: 0.9, 90° dipole illumination) manufactured by ASML Holding N.V., while changing the exposure dose and focus. After the exposure, PEB was performed using a hot plate at 90° C. for 60 seconds. Subsequently, development was performed with a 2.38 mass % TMAH aqueous solution for 30 seconds, to form a line-and-space (LS) pattern.

The LS patterns after the development were observed with CD-SEM (CG6300) manufactured by Hitachi High-Technologies Corporation, and sensitivity and line width roughness (LWR) were evaluated according to the following methods. The results were shown in Tables 9 to 10.

### [Sensitivity Evaluation]

As the sensitivity, an optimum exposure dose Eₒₚ (mJ/cm²) was determined at which a LS pattern with a line width of 18 nm and a pitch of 36 nm was obtained. The smaller this value, the higher the sensitivity.

### [LWR Evaluation]

The size of the LS pattern obtained by the irradiation at Eₒₚ was measured at ten positions in the longitudinal direction of the line. Based on this result, the triple value (3σ) of the standard deviation (a) was determined as LWR. The smaller the value, the smaller the roughness and the more uniform the line width of the obtained pattern.

In this evaluation, the rating was judged as “Good” when the value is 2.5 nm or less, and judged as “Bad” when the value is more than 2.5 nm.

TABLE 9

| | Resist | Eop | |
|---|---|---|---|
| | composition | (mJ / cm²) | LWR (nm) |
| Example 5-1 | R-7 | 45 | Good (2.4) |
| Example 5-2 | R-8 | 44 | Good (2.4) |
| Example 5-3 | R-9 | 44 | Good (2.3) |
| Example 5-4 | R-10 | 43 | Good (2.2) |
| Example 5-5 | R-11 | 44 | Good (2.1) |
| Example 5-6 | R-12 | 45 | Good (2.4) |
| Example 5-7 | R-13 | 44 | Good (2.2) |
| Example 5-8 | R-14 | 46 | Good (2.3) |
| Example 5-9 | R-15 | 42 | Good (2.4) |
| Example 5-10 | R-16 | 44 | Good (2.3) |
| Example 5-11 | R-17 | 40 | Good (2.4) |
| Example 5-12 | R-18 | 39 | Good (2.3) |
| Example 5-13 | R-19 | 39 | Good (2.2) |
| Example 5-14 | R-20 | 41 | Good (2.4) |
| Example 5-15 | R-21 | 38 | Good (2.1) |
| Example 5-16 | R-22 | 41 | Good (2.3) |
| Example 5-17 | R-23 | 40 | Good (2.3) |
| Example 5-18 | R-24 | 40 | Good (2.2) |
| Example 5-19 | R-25 | 39 | Good (2.3) |
| Example 5-20 | R-26 | 38 | Good (2.4) |
| Example 5-21 | R-27 | 41 | Good (2.3) |
| Example 5-22 | R-28 | 41 | Good (2.3) |
| Example 5-23 | R-29 | 35 | Good (2.3) |
| Example 5-24 | R-30 | 37 | Good (2.3) |
| Example 5-25 | R-31 | 33 | Good (2.2) |
| Example 5-26 | R-32 | 33 | Good (2.4) |
| Example 5-27 | R-33 | 34 | Good (2.3) |
| Example 5-28 | R-34 | 36 | Good (2.3) |
| Example 5-29 | R-35 | 35 | Good (2.4) |
| Example 5-30 | R-36 | 34 | Good (2.3) |
| Example 5-31 | R-37 | 35 | Good (2.2) |
| Example 5-32 | R-38 | 33 | Good (2.4) |
| Example 5-33 | R-39 | 35 | Good (2.3) |
| Example 5-34 | R-40 | 33 | Good (2.2) |
| Example 5-35 | R-41 | 36 | Good (2.3) |
| Example 5-36 | R-42 | 35 | Good (2.1) |
| Example 5-37 | R-43 | 37 | Good (2.3) |
| Example 5-38 | R-44 | 34 | Good (2.3) |
| Example 5-39 | R-45 | 35 | Good (2.2) |
| Example 5-40 | R-46 | 36 | Good (2.3) |
| Example 5-41 | R-47 | 35 | Good (2.2) |
| Example 5-42 | R-48 | 33 | Good (2.4) |

TABLE 10

| | Resist | Eop | |
|---|---|---|---|
| | composition | (mJ / cm²) | LWR (nm) |
| Comparative | RC-7 | 53 | Bad (3.4) |
| Example 5-1 | | | |
| Comparative | RC-8 | 55 | Bad (3.3) |
| Example 5-2 | | | |
| Comparative | RC-9 | 55 | Bad (3.3) |
| Example 5-3 | | | |
| Comparative | RC-10 | 50 | Bad (3.1) |
| Example 5-4 | | | |
| Comparative | RC-11 | 48 | Bad (3.3) |
| Example 5-5 | | | |
| Comparative | RC-12 | 49 | Bad (3.4) |
| Example 5-6 | | | |
| Comparative | RC-13 | 49 | Bad (3.2) |
| Example 5-7 | | | |
| Comparative | RC-14 | 42 | Bad (3.0) |
| Example 5-8 | | | |
| Comparative | RC-15 | 53 | Bad (3.1) |
| Example 5-9 | | | |
| Comparative | RC-16 | 49 | Bad (3.2) |
| Example 5-10 | | | |
| Comparative | RC-17 | 45 | Bad (2.9) |
| Example 5-11 | | | |
| Comparative | RC-18 | 44 | Bad (3.0) |
| Example 5-12 | | | |
| Comparative | RC-19 | 42 | Bad (2.8) |
| Example 5-13 | | | |
| Comparative | RC-20 | 43 | Bad (2.9) |
| Example 5-14 | | | |
| Comparative | RC-21 | 45 | Bad (3.0) |
| Example 5-15 | | | |
| Comparative | RC-22 | 40 | Bad (2.8) |
| Example 5-16 | | | |
| Comparative | RC-23 | 40 | Bad (2.7) |
| Example 5-17 | | | |
| Comparative | RC-24 | 39 | Bad (2.9) |
| Example 5-18 | | | |

The results in Tables 9 and 10 showed that Examples 5-1 to 5-42, which used resist compositions containing the inventive sulfonium salt, had good sensitivity and excellent LWR, and were suitable as materials for EUV-lithography. On the other hand, Comparative Examples 5-1 to 5-18, which used resist compositions containing a sulfonium salt different from that of the present invention, showed poor LWR.

The present description includes the following embodiments.

[1]: A sulfonium salt represented by the following general formula (1),

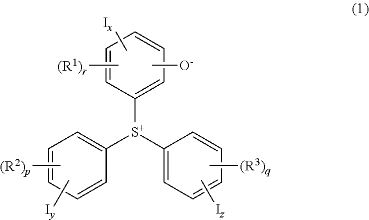

wherein R¹, R², and R³ each independently represent a fluorine atom, a hydroxy group, a trifluoromethyl group, a trifluoromethoxy group, a trifluoroacetyloxy group, a cyano group, a carbamoyl group, an amide group, or a linear, branched, or cyclic monovalent hydrocarbon group having 1 to 15 carbon atoms and optionally having a heteroatom, and a methylene group in the hydrocarbon group may be replaced with an ether bond (—O—) or a carbonyl group (—CO—); R¹ and R² may be bonded to each other to form a ring together with a benzene ring and a sulfur atom, R² and R³ may be bonded to each other to form a ring together with a benzene ring and a sulfur atom, and when the ring is formed, R¹ and R², and R² and R³ may combine to form a single bond, a divalent group, or a divalent atom; “r” represents an integer of 0 to 4, “p” represents an integer of 0 to 5, “q” represents an integer of 0 to 5, “x” represents an integer of 0 to 4, “y” represents an integer of 0 to 5, “z” represents an integer of 0 to 5, and “r”, “p”, “q”, “x”, “y”, and “z” satisfy x+y+z≥1, 4≥r+x≥0, 5≥p+y 0, and 5≥q+z≥0; R's may be the same or different from each other when r≥2, R²s may be the same or different from each other when p≥2, and R³s may be the same or different from each other when q≥2.
   [2]: The sulfonium salt of the above [1], wherein the general formula (1) satisfies x≥1.
   [3]: The sulfonium salt of the above [1] or [2], wherein the general formula (1) satisfies y=0 and z=0.
   [4]: The sulfonium salt of any one of the above [1] to [3], wherein the sulfonium salt is represented by the following general formula (1-A),

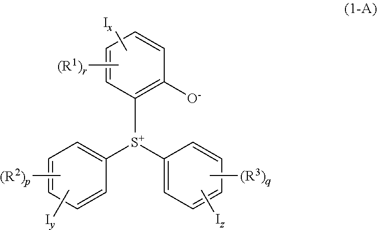

wherein R¹, R², R³, “r”, “p”, “q”, “x”, “y”, and “z” are the same as defined above.
   [5]: The sulfonium salt of any one of the above [1] to [4], wherein R¹, R², and R³ each independently represents a group selected from the group consisting of a fluorine atom, a hydroxy group, a cyano group, a carbamoyl group, or an alkyl group having 1 to 5 carbon atoms and optionally having a heteroatom, an alkoxy group having 1 to 5 carbon atoms and optionally having a heteroatom, and an alkylcarbonyloxy group having 1 to 5 carbon atoms and optionally having a heteroatom.
   [6]: An acid diffusion inhibitor comprising the sulfonium salt of any one of the above [1] to [5].
   [7]: A resist composition comprising a resin component (component A) that changes its solubility in a developer by an action of an acid, a photo-acid generator (component B), an organic solvent (component D), and the acid diffusion inhibitor (component C-1) of the above [6].
   [8]: A resist composition comprising a resin component (component A-1) that generates an acid by exposure to light and changes its solubility in a developer by an action of an acid, an organic solvent (component D), and the acid diffusion inhibitor (component C-1) of the above [6].
   [9]: The resist composition of the above [7] or [8], wherein the resin component (component A) or the resin component (component A-1) has a repeating unit (P-1) having a phenolic hydroxy group.
   [10]: The resist composition of the above [9], wherein the repeating unit (P-1) is represented by the following general formula (5),

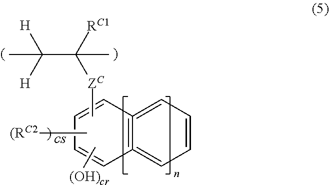

wherein R^{C1} represents a hydrogen atom or a methyl group; Z^{C} represents a single bond or an ester bond; R^{C2} represents a fluorine atom, an iodine atom, a trifluoromethyl group, a trifluoromethoxy group, or a monovalent organic group having 1 to 10 carbon atoms and optionally having a heteroatom, and “—CH₂—” in the organic group may be substituted with “—O—”, or “—C(═O)—”; cs represents an integer of 0 to 4; cr represents an integer of 1 to 5; and “n” represents 0 or 1.
   [11]: The resist composition of any one of the above [7] to [10], comprising an acid diffusion inhibitor (component C-2), other than the acid diffusion inhibitor (component C-1).
   [12]: The resist composition of any one of the above [7] to [11], wherein the resist composition is used to form an image by exposure with an electron beam or EUV light.
   [13]: A patterning process comprising; applying the resist composition of any one of the above [7] to [12] onto a substrate;
heat-treating the resist composition applied on the substrate;
exposing the resist composition to light of a KrF excimer laser beam, an ArF excimer laser beam, an electron beam, or EUV light; and developing the resist composition with a developer.
   [14]: The patterning process of the above [13], wherein an exposed portion is dissolved by using an alkali aqueous solution as the developer, and an unexposed portion is not dissolved to obtain a positive pattern.
   [15]: The patterning process of the above [13], wherein an unexposed portion is dissolved by using an organic solvent as the developer, and an exposed portion is not dissolved to obtain a negative pattern.

It should be noted that the present invention is not limited to the above-described embodiments. The embodiments are just examples, and any examples that have substantially the same feature and demonstrate the same functions and effects as those in the technical concept disclosed in claims of the present invention are included in the technical scope of the present invention.

1. A sulfonium salt represented by the following general formula (1), 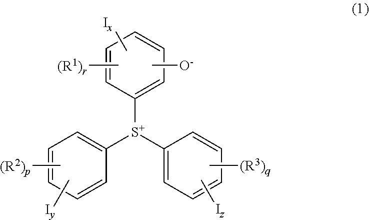
wherein R¹, R², and R³ each independently represent a fluorine atom, a hydroxy group, a trifluoromethyl group, a trifluoromethoxy group, a trifluoroacetyloxy group, a cyano group, a carbamoyl group, an amide group, or a linear, branched, or cyclic monovalent hydrocarbon group having 1 to 15 carbon atoms and optionally having a heteroatom, and a methylene group in the hydrocarbon group may be replaced with an ether bond (—O—) or a carbonyl group (—CO—); R¹ and R² may be bonded to each other to form a ring together with a benzene ring and a sulfur atom, R² and R³ may be bonded to each other to form a ring together with a benzene ring and a sulfur atom, and when the ring is formed, R¹ and R², and R² and R³ may combine to form a single bond, a divalent group, or a divalent atom; “r” represents an integer of 0 to 4, “p” represents an integer of 0 to 5, “q” represents an integer of 0 to 5, “x” represents an integer of 0 to 4, “y” represents an integer of 0 to 5, “z” represents an integer of 0 to 5, and “r”, “p”, “q”, “x”, “y”, and “z” satisfy x+y+z≥1, 4≥r+x≥0, 5≥p+y≥0, and 5≥q+z≥0; R²s may be the same or different from each other when r≥2, R²s may be the same or different from each other when p≥2, and R³s may be the same or different from each other when q≥2.

2. The sulfonium salt according to claim 1, wherein the general formula (1) satisfies x≥1.

3. The sulfonium salt according to claim 2, wherein the general formula (1) satisfies y=0 and z=0.

4. The sulfonium salt according to claim 1, wherein the sulfonium salt is represented by the following general formula (1-A), 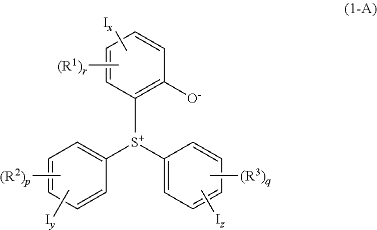
wherein R¹, R², R³, “r”, “p”, “q”, “x”, “y”, and “z” are the same as defined above.

5. The sulfonium salt according to claim 1, wherein R¹, R², and R³ each independently represents a group selected from the group consisting of a fluorine atom, a hydroxy group, a cyano group, a carbamoyl group, or an alkyl group having 1 to 5 carbon atoms and optionally having a heteroatom, an alkoxy group having 1 to 5 carbon atoms and optionally having a heteroatom, and an alkylcarbonyloxy group having 1 to 5 carbon atoms and optionally having a heteroatom.

6. An acid diffusion inhibitor comprising the sulfonium salt according to claim 1.

7. A resist composition comprising a resin component (component A) that changes its solubility in a developer by an action of an acid, a photo-acid generator (component B), an organic solvent (component D), and the acid diffusion inhibitor (component C-1) according to claim 6.

8. A resist composition comprising a resin component (component A-1) that generates an acid by exposure to light and changes its solubility in a developer by an action of an acid, an organic solvent (component D), and the acid diffusion inhibitor (component C-1) according to claim 6.

9. The resist composition according to claim 7, wherein the resin component (component A) has a repeating unit (P-1) having a phenolic hydroxy group.

10. The resist composition according to claim 8, wherein the resin component (component A-1) has a repeating unit (P-1) having a phenolic hydroxy group.

11. The resist composition according to claim 9, wherein the repeating unit (P-1) is represented by the following general formula (5), 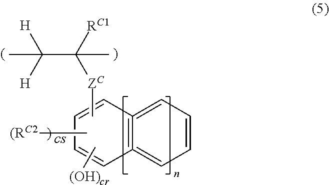
wherein R^{C1} represents a hydrogen atom or a methyl group; Z^{C} represents a single bond or an ester bond; R^{C2} represents a fluorine atom, an iodine atom, a trifluoromethyl group, a trifluoromethoxy group, or a monovalent organic group having 1 to 10 carbon atoms and optionally having a heteroatom, and “—CH₂—” in the organic group may be substituted with “—O—”, or “—C(═O)—”; cs represents an integer of 0 to 4; cr represents an integer of 1 to 5; and “n” represents 0 or 1.

12. The resist composition according to claim 10, wherein the repeating unit (P-1) is represented by the following general formula (5), 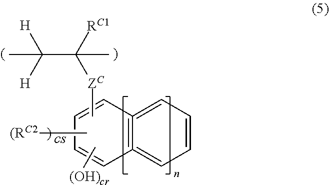
wherein R^{C1} represents a hydrogen atom or a methyl group; Z^{C} represents a single bond or an ester bond; R^{C2} represents a fluorine atom, an iodine atom, a trifluoromethyl group, a trifluoromethoxy group, or a monovalent organic group having 1 to 10 carbon atoms and optionally having a heteroatom, and “—CH₂—” in the organic group may be substituted with “—O—”, or “—C(═O)—”; cs represents an integer of 0 to 4; cr represents an integer of 1 to 5; and “n” represents 0 or 1.

13. The resist composition according to claim 7, comprising an acid diffusion inhibitor (component C-2), other than the acid diffusion inhibitor (component C-1).

14. The resist composition according to claim 7, wherein the resist composition is used to form an image by exposure with an electron beam or EUV light.

15. A patterning process comprising;
applying the resist composition according to claim 7 onto a substrate;
heat-treating the resist composition applied on the substrate;
exposing the resist composition to light of a KrF excimer laser beam, an ArF excimer laser beam, an electron beam, or EUV light; and
developing the resist composition with a developer.

16. A patterning process comprising;
applying the resist composition according to claim 8 onto a substrate;
heat-treating the resist composition applied on the substrate;
exposing the resist composition to light of a KrF excimer laser beam, an ArF excimer laser beam, an electron beam, or EUV light; and
developing the resist composition with a developer.

17. The patterning process according to claim 15, wherein an exposed portion is dissolved by using an alkali aqueous solution as the developer, and an unexposed portion is not dissolved to obtain a positive pattern.

18. The patterning process according to claim 16, wherein an exposed portion is dissolved by using an alkali aqueous solution as the developer, and an unexposed portion is not dissolved to obtain a positive pattern.

19. The patterning process according to claim 15, wherein an unexposed portion is dissolved by using an organic solvent as the developer, and an exposed portion is not dissolved to obtain a negative pattern.

20. The patterning process according to claim 16, wherein an unexposed portion is dissolved by using an organic solvent as the developer, and an exposed portion is not dissolved to obtain a negative pattern.
